(19) United States
(12) Patent Application Publication
(10) Pub. No.: US 2025/0099635 A1
(43) Pub. Date: Mar. 27, 2025

(54) IIMMUNOCONJUGATES COMPRISING KALLIKREIN RELATED PEPTIDASE 2 ANTIGEN BINDING DOMAINS AND THEIR USES

(71) Applicant: Janssen Biotech, Inc., Horsham, PA (US)

(72) Inventors: Fei SHEN, Collegeville, PA (US); Theresa MCDEVITT, Warminster, PA (US); Shalom GOLDBERG, Merion Station, PA (US); Kristen WILLEY, Trappe, PA (US); Ryan M. SMITH, Phoenixville, PA (US); Sathyadevi VENKATARAMANI, Blue Bell, PA (US)

(21) Appl. No.: 18/832,652

(22) PCT Filed: Jan. 25, 2023
(86) PCT No.: PCT/IB2023/050634

§ 371 Date: Jul. 24, 2024

Related U.S. Application Data
(60) Provisional application No. 63/303,083, filed on Jan. 26, 2022.

(51) Int. Cl. A61K 51/10

(52) U.S. Cl. CPC A61K 51/1075; A61K 51/1096; A61K 2123/00

(57) ABSTRACT

Provided herein are immunoconjugates, such as radioimmunoconjugates, comprising a therapeutic moiety conjugated to an antibody or antigen binding domain with binding specificity for hK2. In certain embodiments, the hK2-specific immunoconjugate demonstrates a short half-life. Also provided herein are methods of using the immunoconjugates for selectively targeting cancer cells and for treating diseases such as prostate cancer.

### CROSS REFERENCE TO RELATED APPLICATIONS

This application claims the benefit of priority of U.S. Provisional Application No. 63/303,083, filed on Jan. 26, 2022, which is incorporated by reference herein, in its entirety and for all purposes.

### REFERENCE TO SEQUENCE LISTING SUBMITTED ELECTRONICALLY

This application contains a computer readable Sequence Listing which has been submitted in XML file format with this application, the entire content of which is incorporated by reference herein in its entirety. The Sequence Listing XML file submitted with this application is entitled “JBI6697WOPCT1_SL.xml”, was created on Nov. 7, 2022 and is 747,970 bytes in size.

### TECHNICAL FIELD

The invention provides immunoconjugates, such as radioconjugates, comprising antigen binding domains that bind kallikrein related peptidase 2 (hK2) protein, and methods of making and using them.

### BACKGROUND OF THE INVENTION

Prostate cancer is the second most frequently diagnosed cancer and the sixth leading cause of cancer death in males, accounting for about 14% of the total new cancer cases and about 6% of the total cancer deaths in males worldwide. The course of prostate cancer from diagnosis to death is best categorized as a series of clinical stages based on the extent of disease, hormonal status, and absence or presence of detectable metastases: localized disease, rising levels of prostate-specific antigen (PSA) after radiation therapy or surgery with no detectable metastases, and clinical metastases in the non-castrate or castrate stage. Although surgery, radiation, or a combination of both can be curative for patients with localized disease, a significant proportion of these patients have recurrent disease as evidenced by a rising level of PSA, which can lead to the development of metastases, especially in the high-risk group—a transition to the lethal stage of the disease.

Androgen depletion therapy (ADT) is the standard treatment with a generally predictable outcome: decline in PSA, a period of stability in which the tumor does not proliferate, followed by rising PSA and regrowth as castration-resistant disease. Historically, ADT has been the standard of care for patients with metastatic prostate cancer.

Kallikrein related peptidase 2 (hK2, HK2) is a trypsin-like enzyme with androgen receptor (AR)-driven expression specific to prostate tissue and prostate cancer. hK2 expression is restricted to the prostate and prostate cancer tissue, however it has recently been demonstrated that hK2 was detectable in breast cancer lines and primary patient samples after appropriate activation of the AR-pathway by steroid hormones (U.S. Pat. Publ. No. 2018/0326102). Retrograde release of catalytically inactive hK2 into the blood occurs when the highly structured organization of the prostate is compromised upon hypertrophy or malignant transformation.

There remains a need for next generation hK2-targeted therapies for therapeutic and diagnostic purposes.

### BRIEF SUMMARY OF THE INVENTION

Embodiments of the present invention relate to an anti-hk2 radioconjugate comprising an antigen binding domain conjugated with a chelator that binds radiometals for therapeutic use or imaging. According to particular embodiments, the anti-hK2 radioconjugate comprising an antigen binding domain has a shorter half-life compared to an anti-hK2 radioconjugate comprising a full-length antibody.

In many cases the circulating half-life of immunoglobulin G (IgG) in humans is approximately 10-21 days. The Fc domain in an intact IgG is capable of binding to the neonatal Fc receptor (FcRn), leading to antibody recycling and minimal endosomal degradation. FcRn plays a key role in serum IgG homeostasis as well as in placental transfer of IgG molecules from mother to fetus. Following pinocytosis, the acidic environment of the early endosome allows for binding of IgG (as well as albumin) to FcRn, which provides protection from degradation and facilitates trafficking of IgG back to the extracellular environment, where the molecules dissociate back into circulation upon exposure to physiological pH.

The circulating half-life of an antigen binding domain, such as a Fab, tends to be much shorter than that of an IgG. As the Fab fragment lacks the Fc domain, the FcRn mediated enhanced half-life mechanism is lacking, thus the Fab alone has a shorter half-life (for example, less than 24 hours, or less than 12 hours, and in some cases about 2-3 hours).

An embodiment of the present invention provides immunoconjugate comprising a therapeutic moiety conjugated to an antigen binding domain with binding specificity for kallikrein related peptidase 2 (hK2).

According to certain embodiments, the therapeutic moiety is a cytotoxic agent.

According to certain embodiments, the therapeutic moiety is an imaging agent.

According to certain embodiments, the therapeutic moiety comprises a radiometal. Non-limiting examples of suitable radiometals include ²²⁵Ac, ¹⁷⁷Lu, ³²P, ⁴⁷Sc, ⁶⁷Cu, ⁷⁷As, ⁸⁹Sr, ⁹⁰Y, ⁹⁹Tc, ¹⁰⁵Rh, ¹⁰⁹Pd, ¹¹¹Ag, ¹³¹I, ¹⁴⁹Tb, ¹⁵²Tb, ¹⁵⁵Tb, ¹⁵³Sm, ¹⁵⁹Gd, ¹⁶⁵Dy, ¹⁶⁶Ho, ¹⁶⁹Er, ¹⁸⁶Re, ¹⁸⁸Re, ¹⁹⁴Ir, ¹⁹⁸Au, ¹⁹⁹Au, ²¹¹At, ²¹²Pb, ²¹²Bi, ²¹³Bi, ²²³Ra, ²⁵⁵Fm, ²²⁷Th, ¹⁷⁷Lu, ⁶²Cu, ⁶⁴Cu, ⁶⁷Ga, ⁶⁸Ga, ⁸⁶Y, ⁸⁹Zr, and ¹¹¹In.

According to certain embodiments, the therapeutic moiety is a cytotoxic agent comprising ²²⁵Ac.

According to certain embodiments, the therapeutic moiety is an imaging agent comprising ¹¹¹In or ⁶⁴Cu.

According to certain embodiments, the therapeutic moiety comprises a radiometal complex, wherein the radiometal complex comprises the radiometal bound to a chelator, and wherein the chelator is conjugated to the antigen binding domain with binding specificity for kallikrein related peptidase 2 (hK2).

According to certain embodiments, the chelator is 1,4,7,10-tetraazacyclododecane-1,4,7,10,tetraacetic acid (DOTA), S-2-(4-isothiocyanatobenzyl)-1,4,7-triazacyclononane-1,4,7-triacetic acid (NOTA), 1,4,8,11-tetraazacyclodocedan-1,4,8,11-tetraacetic acid (TETA), 3,6,9,15-tetraazabicyclo[9.3.1]-pentadeca-1(15),11,13-triene-4-(S)-(4-isothiocyanatobenzyl)-3,6,9-triacetic acid (PCTA), 5-S-(4-aminobenzyl)-1-oxa-4,7,10-triazacyclododecane-4,7,10-tris(acetic acid) (DO3A), or a derivative thereof.

According to certain embodiments, the chelator is DOTA.

According to certain embodiments, the chelator is H₂bp18c6 or a H₂bp18c6 derivative.

According to certain embodiments, the radiometal complex is a radiocomplex of Formula (I-m), or Formula (II-m), or Formula (III-m) as described herein, wherein R₁₁ comprises the antigen binding domain with binding specificity for kallikrein related peptidase 2 (hK2) and M is the radiometal.

According to certain embodiments, the radiometal complex is a radiometal complex of Formula (IV-m), or Formula (V-m), or Formula (VI-m) as described herein, wherein R₄ comprises the antigen binding domain with binding specificity for kallikrein related peptidase 2 (hK2) and M+ is the radiometal.

According to certain embodiments, the therapeutic moiety is an auristatin derivative, such as MMAE (monomethyl auristatin E) or MMAF (monomethyl auristatin F).

According to certain embodiments, the antigen binding domain that binds hK2 is a scFv, a (scFv)₂, a Fv, a Fab, a F(ab′)₂, a Fd, a dAb or a VHH.

According to certain embodiments, the antigen binding domain with binding specificity for hK2 is a Fab.

According to certain embodiments, the antigen binding domain comprises the HCDR1, the HCDR2, the HCDR3, the LCDR1, the LCDR2 and the LCDR3 of SEQ ID NO: 170 (SYYWS), SEQ ID NO: 171 (YIYYSGSTNYNPSLKS), SEQ ID NO: 172 (TTIFGVVTPNFYYGMDV), SEQ ID NO: 173 (RASQGISSYLA), SEQ ID NO: 174 (AASTLQS) and SEQ ID NO: 175 (QQLNSYPLT), respectively.

According to certain embodiments, the antigen binding domain that binds hK2 comprises a VH which is at least 80% (e.g. at least 85%, at least 90%, at least 95%, at least 99% or 100%) identical to the VH of SEQ ID NO: 162 (QVQLQESGPGLVKPSETLSLTCTVSGGSISSYYWSWIRQPPGKGLEWIGYIYYSGSTNYNPSL KSRVTISVDTSKNQFSLKLSSVTAADTAVYYCAGTTIFGVVTPNFYYGMDVWGQGTTVTVS S), and a VL which is at least 80% (e.g. at least 85%, at least 90%, at least 95%, at least 99% or 100%) identical to the VL of SEQ ID NO: 163 (DIQMTQSPSFLSASVGDRVTITCRASQGISSYLAWYQQKPGKAPKFLIYAASTLQSGVPSRFS GSGSGTEFTLTISSLQPEDFATYYCQQLNSYPLTFGGGTKVEIK).

According to certain embodiments, the antigen binding domain that binds hK2 comprises the VH of SEQ ID NO: 162 and the VL of SEQ ID NO: 163.

According to certain embodiments, the antigen binding domain is a Fab that comprises: A) a HCDR1, a HCDR2, a HCDR3, a LCDR1, a LCDR2 and a LCDR3 of SEQ ID NOs: 170, 171, 172, 173, 174 and 175, respectively; and/or B) a VH of SEQ ID NO: 162 and a VL of SEQ ID NO: 163.

According to certain embodiments, the immunoconjugate is a short half-life immunoconjugate.

According to certain embodiments, a method of treating an hK2-expressing cancer in a subject, comprises administering to the subject a therapeutically effective amount of the immunoconjugate according to any of the foregoing embodiments.

According to certain embodiments, a method of reducing the amount of hK2-expressing tumor cells in a subject, comprises administering to the subject a therapeutically effective amount of the immunoconjugate according to any of the foregoing embodiments.

According to certain embodiments, a method of treating prostate cancer in a subject comprises administering to the subject a therapeutically effective amount of the immunoconjugate according to any of the foregoing embodiments.

According to certain embodiments, the prostate cancer is relapsed, refractory, malignant or castration resistant prostate cancer, or any combination thereof.

According to certain embodiments, the prostate cancer is metastatic castration-resistant prostate cancer.

According to certain embodiments, a method of detecting the presence of prostate cancer in a subject, comprising administering the immunoconjugate according to any of the foregoing embodiments to a subject suspected to have prostate cancer and visualizing the biological structures to which the conjugate is bound (e.g., by computerized tomography or positron emission tomography), thereby detecting the presence of prostate cancer, wherein the immunoconjugate preferably comprises an imaging agent, such as 111-In or 64-Cu. According to certain embodiments, the method comprises conjugating the therapeutic moiety to the antigen binding domain with binding specificity for kallikrein related peptidase 2 (hK2).

According to certain embodiments, a method of making a radioimmunoconjugate as described herein comprises binding a radiometal to a chelator that is conjugated to an antigen binding domain with binding specificity for kallikrein related peptidase 2 (hK2).

According to certain embodiments, a short half-life radioimmunoconjugate comprises a radiometal complex, wherein the radiometal complex comprises ¹¹⁵Ac bound to a chelator, and wherein the chelator is conjugated to a Fab with binding specificity for hK2, said Fab comprising: a HCDR1, a HCDR2, a HCDR3, a LCDR1, a LCDR2 and a LCDR3 of SEQ ID NOs: 170, 171, 172, 173, 174 and 175, respectively. According to certain embodiments, said Fab comprises a VH of SEQ ID NO: 162 and a VL of SEQ ID NO: 163.

### DETAILED DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

In the drawings:

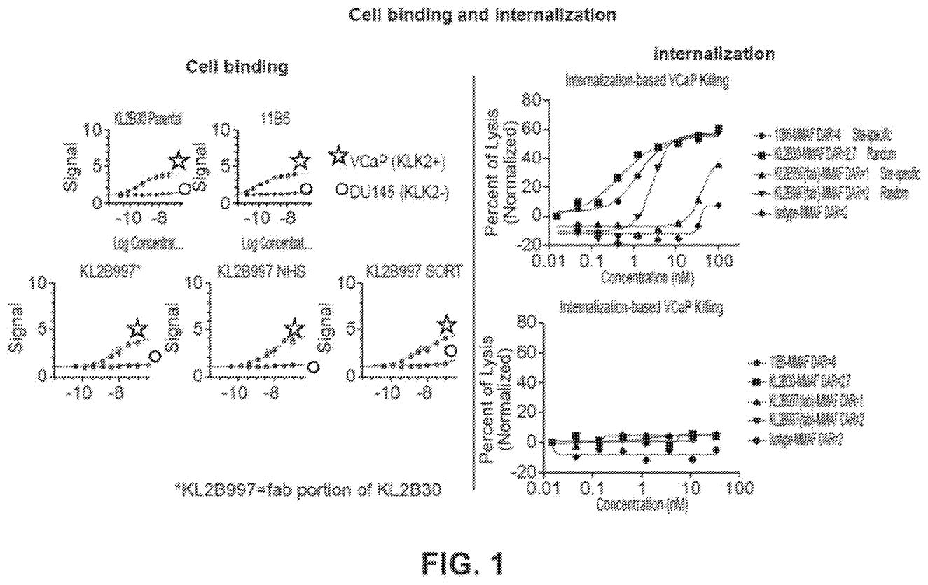

FIG. 1 shows cell binding and internalization of an immunoconjugate of the present invention, comprising the KL2B30 Fab (identified as KL2B997) conjugated to MMAF (monomethyl auristatin F), in hK2-expressing VCaP cells.

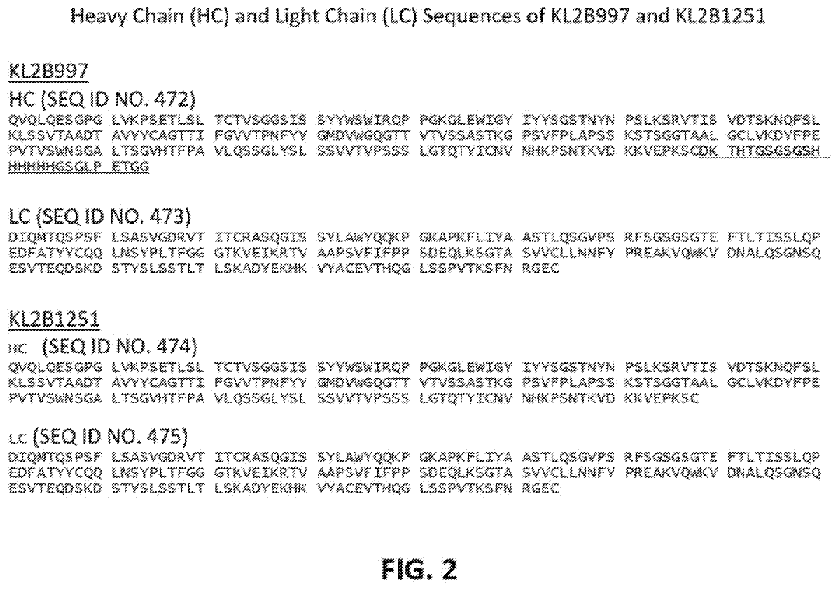

FIG. 2 shows amino acid sequences (heavy chain and light chain sequences) of KL2B997 and KL2B1251. KL2B997 has a His-tag and a sortase tag (underlined in FIG. 2) on the heavy chain, and KL2B1251 is tagless.

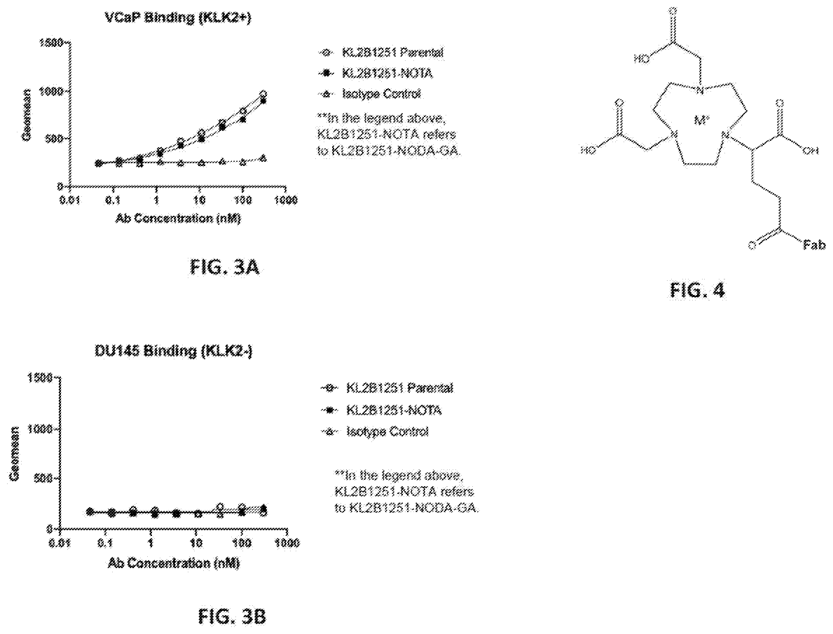

FIG. 3A shows cell binding of a KL2B30 Fab (KL2B1251) and KL2B1251 conjugated to a NOTA derivative (NODA-GA) in hK2-expressing VCaP cells; and FIG. 3B shows lack of binding in hk2-negative DU145 cells.

FIG. 4 shows a representative structure of a NODA-GA-Fab according to an embodiment of the present invention.

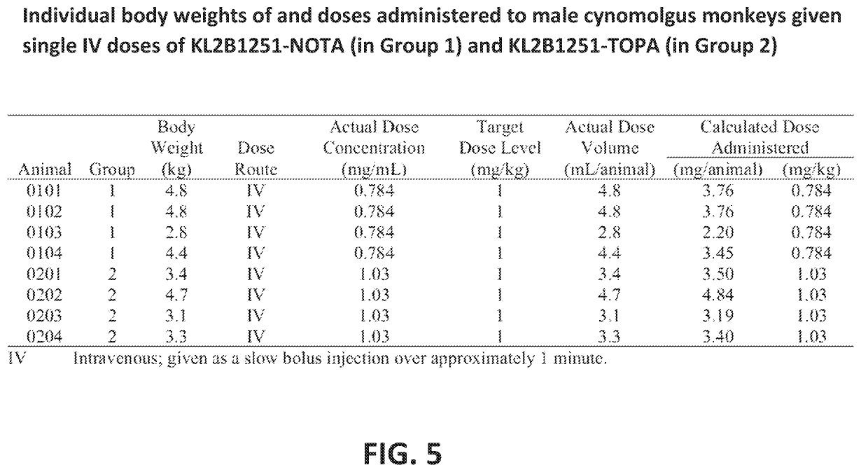

FIG. 5 shows individual body weights of and doses administered to male cynomolgus monkeys given single IV doses of KL2B1251-NOTA (in Group 1) and KL2B1251-TOPA (in Group 2).

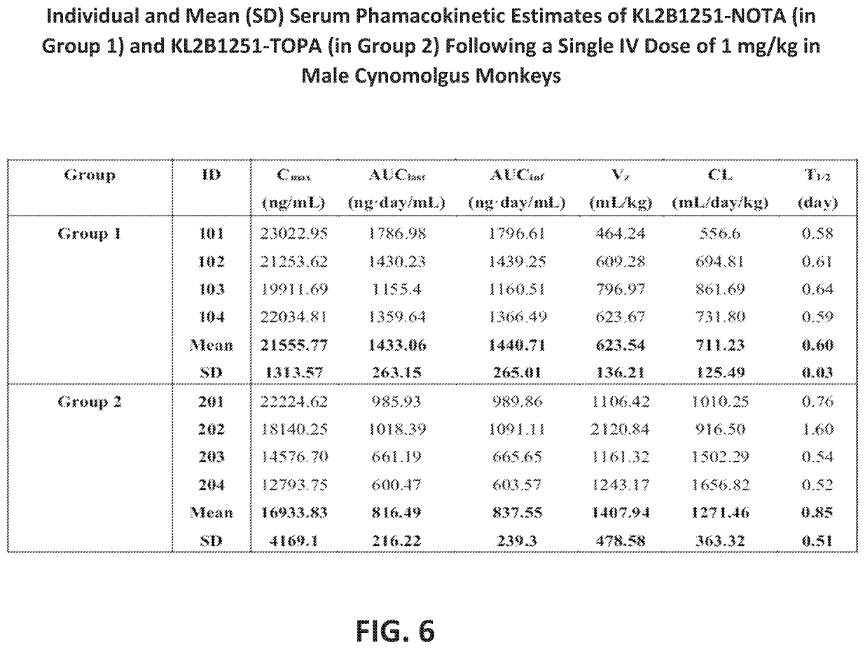

FIG. 6 shows individual and mean (SD) serum pharmacokinetic estimates of KL2B1251-NOTA (in Group 1) and KL2B1251-TOPA (in Group 2) following a single IV dose of 1 mg/kg in male cynomolgus monkeys.

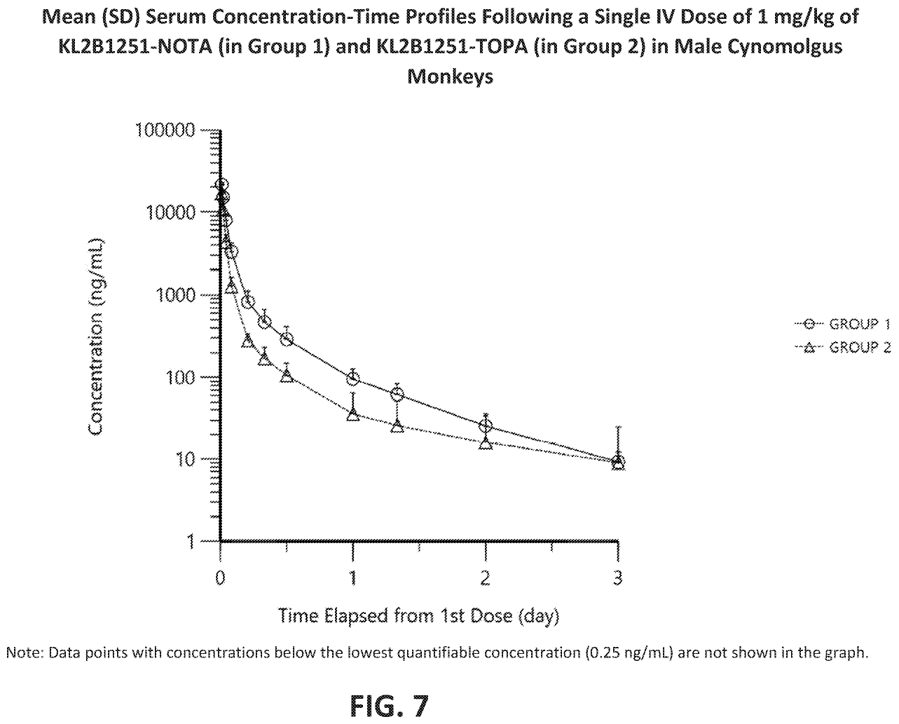

FIG. 7 shows mean (SD) serum concentrations of KL2B1251-NOTA and KL2B1251-TOPA versus time following a single IV dose of 1 mg/kg in male cynomolgus monkeys.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning commonly understood to one of ordinary skill in the art to which this invention pertains. Otherwise, certain terms cited herein have the meanings as set in the specification. All patents, published patent applications and publications cited herein are incorporated by reference as if set forth fully herein

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

In an attempt to help the reader of the application, the description has been separated into various paragraphs or sections, or is directed to various embodiments of the application. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples.

As used herein, the use of a numerical range expressly includes all possible subranges, all individual numerical values within that range, including integers within such ranges and fractions of the values unless the context clearly indicates otherwise.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used in this specification and the appended claims, the singular forms “a,” “an,” and “the” include plural referents unless the content clearly dictates otherwise. Thus, for example, reference to “a cell” includes a combination of two or more cells, and the like.

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents) also provide as embodiments those independently described in terms of “consisting of” and “consisting essentially of.”

“About” means within an acceptable error range for the particular value as determined by one of ordinary skill in the art, which will depend in part on how the value is measured or determined, i.e., the limitations of the measurement system.

“Antibody-dependent cellular cytotoxicity”, “antibody-dependent cell-mediated cytotoxicity” or “ADCC” refers to the mechanism of inducing cell death that depends upon the interaction of antibody-coated target cells with effector cells possessing lytic activity, such as natural killer cells (NK), monocytes, macrophages and neutrophils via Fc gamma receptors (FcγR) expressed on effector cells.

“Antibody-dependent cellular phagocytosis” or “ADCP” refers to the mechanism of elimination of antibody-coated target cells by internalization by phagocytic cells, such as macrophages or dendritic cells.

“Antigen” refers to any molecule (e.g., protein, peptide, polysaccharide, glycoprotein, glycolipid, nucleic acid, portions thereof, or combinations thereof) capable of being bound by an antigen binding domain or a T-cell receptor capable of mediating an immune response. Exemplary immune responses include antibody production and activation of immune cells, such as T cells, B cells or NK cells. Antigens may be expressed by genes, synthetized, or purified from biological samples such as a tissue sample, a tumor sample, a cell or a fluid with other biological components, organisms, subunits of proteins/antigens, killed or inactivated whole cells or lysates.

“Antigen binding fragment” or “antigen binding domain” refers to a portion of an isolated protein that binds an antigen. Antigen binding fragments may be synthetic, enzymatically obtainable or genetically engineered polypeptides and include portions of an immunoglobulin that bind an antigen, such as the VH, the VL, the VH and the VL, Fab, Fab′, F(ab′)₂, Fd and Fv fragments, domain antibodies (dAb) consisting of one VH domain or one VL domain, shark variable IgNAR domains, camelized VH domains, VHH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3, alternative scaffolds that bind an antigen, and multispecific proteins comprising the antigen binding fragments. Antigen binding fragments (such as VH and VL) may be linked together via a synthetic linker to form various types of single antibody designs where the VH/VL domains may pair intramolecularly, or intermolecularly in those cases when the VH and VL domains are expressed by separate single chains, to form a monovalent antigen binding domain, such as single chain Fv (scFv) or diabody. As used herein, an “antigen binding fragment” or “antigen binding domain” does not refer to a full-length antibody having an Fc region.

“Antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific, dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g. IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4. Immunoglobulins may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

“Cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor.

“Complementarity determining regions” (CDR) are antibody regions that bind an antigen. There are three CDRs in the VH (HCDR1, HCDR2, HCDR3) and three CDRs in the VL (LCDR1, LCDR2, LCDR3). CDRs may be defined using various delineations such as Kabat (Wu et al. (1970) J Exp Med 132: 211-50; Kabat et al., Sequences of Proteins of Immunological Interest, 5th Ed. Public Health Service, National Institutes of Health, Bethesda, Md., 1991), Chothia (Chothia et al. (1987) J Mol Biol 196: 901-17), IMGT (Lefranc et al. (2003) Dev Comp Immunol 27: 55-77) and AbM (Martin and Thornton J Bmol Biol 263: 800-15, 1996). The correspondence between the various delineations and variable region numbering is described (see e.g. Lefranc et al. (2003) Dev Comp Immunol 27: 55-77; Honegger and Pluckthun, J Mol Biol (2001) 309:657-70; International ImMunoGeneTics (IMGT) database; Web resources, http://www_imgt_org). Available programs such as abYsis by UCL Business PLC may be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification.

“Decrease,” “lower,” “lessen,” “reduce,” or “abate” refers generally to the ability of a test molecule to mediate a reduced response (i.e., downstream effect) when compared to the response mediated by a control or a vehicle. Exemplary responses are T cell expansion, T cell activation or T-cell mediated tumor cell killing or binding of a protein to its antigen or receptor, enhanced binding to a Fcγ or enhanced Fc effector functions such as enhanced ADCC, CDC and/or ADCP. Decrease may be a statistically significant difference in the measured response between the test molecule and the control (or the vehicle), or a decrease in the measured response, such as a decrease of about 1.1, 1.2, 1.5, 2, 3, 4, 5, 6, 7, 8, 9, 10, 15, 20 or 30 fold or more, such as 500, 600, 700, 800, 900 or 1000 fold or more (including all integers and decimal points in between and above 1, e.g., 1.5, 1.6, 1.7. 1.8, etc.).

“Differentiation” refers to a method of decreasing the potency or proliferation of a cell or moving the cell to a more developmentally restricted state.

“Encode” or “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence, and the non-coding strand, used as the template for transcription of a gene or cDNA, can be referred to as encoding the protein or other product of that gene or cDNA.

“Enhance,” “promote,” “increase,” “expand” or “improve” refers generally to the ability of a test molecule to mediate a greater response (i.e., downstream effect) when compared to the response mediated by a control or a vehicle. Exemplary responses are T cell expansion, T cell activation or T-cell mediated tumor cell killing or binding of a protein to its antigen or receptor, enhanced binding to a Fcγ or enhanced Fc effector functions such as enhanced ADCC, CDC and/or ADCP. Enhance may be a statistically significant difference in the measured response between the test molecule and control (or vehicle), or an increase in the measured response, such as an increase of about 1.1, 1.2, 1.5, 2, 3, 4, 5, 6, 7, 8, 9, 10, 15, 20 or 30 fold or more, such as 500, 600, 700, 800, 900 or 1000 fold or more (including all integers and decimal points in between and above 1, e.g., 1.5, 1.6, 1.7. 1.8, etc.).

“Epitope” refers to a portion of an antigen to which an antibody specifically binds. Epitopes typically consist of chemically active (such as polar, non-polar or hydrophobic) surface groupings of moieties such as amino acids or polysaccharide side chains and may have specific three-dimensional structural characteristics, as well as specific charge characteristics. An epitope may be composed of contiguous and/or discontinuous amino acids that form a conformational spatial unit. For a discontinuous epitope, amino acids from differing portions of the linear sequence of the antigen come in close proximity in 3-dimensional space through the folding of the protein molecule. Antibody “epitope” depends on the methodology used to identify the epitope.

“Expansion” refers to the outcome of cell division and cell death.

“Express” and “expression” refers the to the well-known transcription and translation occurring in cells or in vitro. The expression product, e.g., the protein, is thus expressed by the cell or in vitro and may be an intracellular, extracellular or a transmembrane protein.

“Expression vector” refers to a vector that can be utilized in a biological system or in a reconstituted biological system to direct the translation of a polypeptide encoded by a polynucleotide sequence present in the expression vector.

“dAb” or “dAb fragment” refers to an antibody fragment composed of a VH domain (Ward et al., Nature 341:544 546 (1989)).

“Fab” or “Fab fragment” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders.

“F(ab′)₂” or “F(ab′)₂ fragment” refers to an antibody fragment containing two Fab fragments connected by a disulfide bridge in the hinge region.

“Fd” or “Fd fragment” refers to an antibody fragment composed of VH and CH1 domains.

“Fv” or “Fv fragment” refers to an antibody fragment composed of the VH and the VL domains from a single arm of the antibody. Fv fragments lack the constant regions of Fab (CH1 and CL) regions. The VH and VL in Fv fragments are held together by non-covalent interactions.

“Fc“polypeptide” of a dimeric Fc refers to one of the two polypeptide forming the dimeric Fc domain. For example, an Fc polypeptide of a dimeric IgG FC comprises an IgG CH2 and an IgG CH3 constant domain sequence).

“Full length antibody” is comprised of two heavy chains (HC) and two light chains (LC) inter-connected by disulfide bonds as well as multimers thereof (e.g. IgM). Each heavy chain is comprised of a heavy chain variable domain (VH) and a heavy chain constant domain, the heavy chain constant domain comprised of subdomains CH1, hinge, CH2 and CH3. Each light chain is comprised of a light chain variable domain (VL) and a light chain constant domain (CL). The VH and the VL may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4.

“Host cell” refers to any cell that contains a heterologous nucleic acid. An exemplary heterologous nucleic acid is a vector (e.g., an expression vector).

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” may contain consensus framework sequences derived from human framework sequence analyses, for example as described in Knappik et al., (2000) J Mol Biol 296:57-86, or a synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

“Humanized antibody” refers to an antibody in which at least one CDR is derived from non-human species and at least one framework is derived from human immunoglobulin sequences. Humanized antibody may include substitutions in the frameworks so that the frameworks may not be exact copies of expressed human immunoglobulin or human immunoglobulin germline gene sequences.

“In combination with” means that two or more therapeutic agents are be administered to a subject together in a mixture, concurrently as single agents or sequentially as single agents in any order.

“Isolated” refers to a homogenous population of molecules (such as synthetic polynucleotides or polypeptides) which have been substantially separated and/or purified away from other components of the system the molecules are produced in, such as a recombinant cell, as well as a protein that has been subjected to at least one purification or isolation step.

“Isolated” refers to a molecule that is substantially free of other cellular material and/or chemicals and encompasses molecules that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“Kallikrein related peptidase 2” or “hK2” (also referred to herein as KLK2) refers to a known protein which is also called kallikrein-2, grandular kallikrein 2, or HK2. hK2 is produced as a preproprotein and cleaved during proteolysis to generate active protease. All hK2 isoforms and variants are encompassed in “hK2”. The amino acid sequences of the various isoforms are retrievable from GenBank accession numbers NP 005542.1, NP_001002231.1 and NP_001243009. The amino acid sequence of a full length hK2 is shown in SEQ ID NO: 62. The sequence includes the signal peptide (residues 1-18) and the pro-peptide region (residues 19-24).

SEQ ID NO: 62
MWDLVLSIALSVGCTGAVPLIQSRIVGGWECEKHSQPWQVAVYSHGWAH
CGGVLVHPQWVLTAAHCLKKNSQVWLGRHNLFEPEDTGQRVPVSHSFPH
PLYNMSLLKHQSLRPDEDSSHDLMLLRLSEPAKITDVVKVLGLPTQEPA
LGTTCYASGWGSIEPEEFLRPRSLQCVSLHLLSNDMCARAYSEKVTEFM
LCAGLWTGGKDTCGGDSGGPLVCNGVLQGITSWGPEPCALPEKPAVYTK
VVHYRKWIKDTIAANP

“Modulate” refers to either enhanced or decreased ability of a test molecule to mediate an enhanced or a reduced response (i.e., downstream effect) when compared to the response mediated by a control or a vehicle.

“Monoclonal antibody” refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation. Monoclonal antibodies typically bind one antigenic epitope. A bispecific monoclonal antibody binds two distinct antigenic epitopes. Monoclonal antibodies may have heterogeneous glycosylation within the antibody population. Monoclonal antibody may be monospecific or multispecific such as bispecific, monovalent, bivalent or multivalent.

“Operatively linked” and similar phrases, when used in reference to nucleic acids or amino acids, refers to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA) and in some instances to the production of a polypeptide (i.e., expression of the open reading frame). Operatively linked peptide refers to a peptide in which the functional domains of the peptide are placed with appropriate distance from each other to impart the intended function of each domain.

The term “paratope” refers to the area or region of an antibody molecule which is involved in binding of an antigen and comprise residues that interact with an antigen. A paratope may composed of continuous and/or discontinuous amino acids that form a conformational spatial unit. The paratope for a given antibody can be defined and characterized at different levels of details using a variety of experimental and computational methods. The experimental methods include hydrogen/deuterium exchange mass spectrometry (HX-MS). The paratope will be defined differently depending on the mapping method employed.

“Pharmaceutical combination” refers to a combination of two or more active ingredients administered either together or separately.

“Pharmaceutical composition” refers to a composition that results from combining an active ingredient and a pharmaceutically acceptable carrier.

“Pharmaceutically acceptable carrier” or “excipient” refers to an ingredient in a pharmaceutical composition, other than the active ingredient, which is nontoxic to a subject. Exemplary pharmaceutically acceptable carriers are a buffer, stabilizer or preservative.

“Polynucleotide” or “nucleic acid” refers to a synthetic molecule comprising a chain of nucleotides covalently linked by a sugar-phosphate backbone or other equivalent covalent chemistry. cDNA is a typical example of a polynucleotide. Polynucleotide may be a DNA or a RNA molecule.

“Prevent,” “preventing,” “prevention,” or “prophylaxis” of a disease or disorder means preventing that a disorder occurs in a subject.

“Proliferation” refers to an increase in cell division, either symmetric or asymmetric division of cells.

“Promoter” refers to the minimal sequences required to initiate transcription. Promoter may also include enhancers or repressor elements which enhance or suppress transcription, respectively.

“Protein” or “polypeptide” are used interchangeably herein are refers to a molecule that comprises one or more polypeptides each comprised of at least two amino acid residues linked by a peptide bond. Protein may be a monomer, or may be protein complex of two or more subunits, the subunits being identical or distinct. Small polypeptides of less than 50 amino acids may be referred to as “peptides”. Protein may be a heterologous fusion protein, a glycoprotein, or a protein modified by post-translational modifications such as phosphorylation, acetylation, myristoylation, palmitoylation, glycosylation, oxidation, formylation, amidation, citrullination, polyglutamylation, ADP-ribosylation, pegylation or biotinylation. Protein may be recombinantly expressed.

“Recombinant” refers to polynucleotides, polypeptides, vectors, viruses and other macromolecules that are prepared, expressed, created or isolated by recombinant means.

“Regulatory element” refers to any cis- or trans acting genetic element that controls some aspect of the expression of nucleic acid sequences.

“Relapsed” refers to the return of a disease or the signs and symptoms of a disease after a period of improvement after prior treatment with a therapeutic.

“Refractory” refers to a disease that does not respond to a treatment. A refractory disease can be resistant to a treatment before or at the beginning of the treatment, or a refractory disease can become resistant during a treatment.

“Single chain Fv” or “scFv” refers to a fusion protein comprising at least one antibody fragment comprising a light chain variable region (VL) and at least one antibody fragment comprising a heavy chain variable region (VH), wherein the VL and the VH are contiguously linked via a polypeptide linker, and capable of being expressed as a single chain polypeptide. Unless specified, as used herein, a scFv may have the VL and VH variable regions in either order, e.g., with respect to the N-terminal and C-terminal ends of the polypeptide, the scFv may comprise VL-linker-VH or may comprise VH-linker-VL.

“(scFv)₂” or “tandem scFv” or “bis-scFv” fragments refers to a fusion protein comprising two light chain variable region (VL) and two heavy chain variable region (VH), wherein the two VL and the two VH regions are contiguously linked via polypeptide linkers, and capable of being expressed as a single chain polypeptide. The two VL and two VH regions fused by peptide linkers form a bivalent molecule VL_{A}-linker-VH_{A}-linker-VL_{B}-linker-VH_{B} to form two binding sites, capable of binding two different antigens or epitopes concurrently.

“Specifically binds,” “specific binding,” “specifically binding” or “binds” refer to a protein molecule binding to an antigen or an epitope within the antigen with greater affinity than for other antigens. Typically, the protein molecule binds to the antigen or the epitope within the antigen with an equilibrium dissociation constant (K_{D}) of about 1×10⁻⁷ M or less, for example about 5×10⁻⁸ M or less, about 1×10⁻⁸ M or less, about 1×10⁻⁹ M or less, about 1×10⁻¹⁰ M or less, about 1×10⁻¹¹ M or less, or about 1×10⁻¹² M or less, typically with the K_{D} that is at least one hundred fold less than its K_{D} for binding to a non-specific antigen (e.g., BSA, casein). In the context of the prostate neoantigens described here, “specific binding” refers to binding of the protein molecule to the prostate neoantigen without detectable binding to a wild-type protein the neoantigen is a variant of As used herein, an antibody or antigen binding domain “with binding specificity for hK2” refers to an antibody or antigen binding domain that specifically binds to hK2, respectively.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” can be used interchangeably herein.

“T cell” and “T lymphocyte” are interchangeable and used synonymously herein. T cell includes thymocytes, naïve T lymphocytes, memory T cells, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4⁺ T cell) CD4⁺ T cell, a cytotoxic T cell (CTL; CD8⁺ T cell), a tumor infiltrating cytotoxic T cell (TIL; CD8⁺ T cell), CD4⁺CD8⁺ T cell, or any other subset of T cells. Also included are “NKT cells”, which refer to a specialized population of T cells that express a semi-invariant αβ T-cell receptor, but also express a variety of molecular markers that are typically associated with NK cells, such as NK1.1. NKT cells include NK1.1⁺ and NK1.1⁻, as well as CD4⁺, CD4⁻, CD8⁺ and CD8⁻ cells. The TCR on NKT cells is unique in that it recognizes glycolipid antigens presented by the MHC I-like molecule CD Id. NKT cells can have either protective or deleterious effects due to their abilities to produce cytokines that promote either inflammation or immune tolerance. Also included are “gamma-delta T cells (γδ T cells),” which refer to a specialized population that to a small subset of T cells possessing a distinct TCR on their surface, and unlike the majority of T cells in which the TCR is composed of two glycoprotein chains designated α- and β-TCR chains, the TCR in γδ T cells is made up of a γ-chain and a δ-chain. γδ T cells can play a role in immunosurveillance and immunoregulation and were found to be an important source of IL-17 and to induce robust CD8⁺ cytotoxic T cell response. Also included are “regulatory T cells” or “Tregs” which refer to T cells that suppress an abnormal or excessive immune response and play a role in immune tolerance. Tregs are typically transcription factor Foxp3-positive CD4+ T cells and can also include transcription factor Foxp3-negative regulatory T cells that are IL-10-producing CD4⁺ T cells.

“Therapeutically effective amount” or “effective amount” used interchangeably herein, refers to an amount effective, at dosages and for periods of time necessary, to achieve a desired therapeutic result. A therapeutically effective amount may vary according to factors such as the disease state, age, sex, and weight of the individual, and the ability of a therapeutic or a combination of therapeutics to elicit a desired response in the individual. Example indicators of an effective therapeutic or combination of therapeutics that include, for example, improved well-being of the patient, reduction of a tumor burden, arrested or slowed growth of a tumor, and/or absence of metastasis of cancer cells to other locations in the body.

“Transduction” refers to the introduction of a foreign nucleic acid into a cell using a viral vector.

“Treat,” “treating” or “treatment” of a disease or disorder such as cancer refers to accomplishing one or more of the following: reducing the severity and/or duration of the disorder, inhibiting worsening of symptoms characteristic of the disorder being treated, limiting or preventing recurrence of the disorder in subjects that have previously had the disorder, or limiting or preventing recurrence of symptoms in subjects that were previously symptomatic for the disorder.

“Tumor cell” or a “cancer cell” refers to a cancerous, pre-cancerous or transformed cell, either in vivo, ex vivo, or in tissue culture, that has spontaneous or induced phenotypic changes. These changes do not necessarily involve the uptake of new genetic material. Although transformation may arise from infection with a transforming virus and incorporation of new genomic nucleic acid, uptake of exogenous nucleic acid or it can also arise spontaneously or following exposure to a carcinogen, thereby mutating an endogenous gene. Transformation/cancer is exemplified by morphological changes, immortalization of cells, aberrant growth control, foci formation, proliferation, malignancy, modulation of tumor specific marker levels, invasiveness, tumor growth in suitable animal hosts such as nude mice, and the like, in vitro, in vivo, and ex vivo.

“Variant,” “mutant” or “altered” refers to a polypeptide or a polynucleotide that differs from a reference polypeptide or a reference polynucleotide by one or more modifications, for example one or more substitutions, insertions or deletions.

The numbering of amino acid residues in the antibody constant region throughout the specification is according to the EU index as described in Kabat et al., Sequences of Proteins of Immunological Interest, 5th Ed. Public Health Service, National Institutes of Health, Bethesda, MD. (1991), unless otherwise explicitly stated.

Mutations in the Ig constant regions are referred to as follows: L351Y_F405A_Y407V refers to L351Y, F405A and Y407V mutations in one immunoglobulin constant region. L351Y_F405A_Y407V/T394W refers to L351Y, F405A and Y407V mutations in the first Ig constant region and T394W mutation in the second Ig constant region, which are present in one multimeric protein.

“VHH” refers to a single-domain antibody or nanobody, exclusively composed of the antigen binding domain of a heavy chain. A VHH single domain antibody lacks the light chain and the CH1 domain of the heavy chain of conventional Fab region.

### Chemical Nomenclature

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology will be readily apparent to those skilled in the art based on the disclosure herein.

The term “substituted” means that at least one hydrogen atom is replaced with a non-hydrogen group, provided that all normal valencies are maintained and that the substitution results in a stable compound. When a particular group is “substituted,” that group can have one or more substituents, preferably from one to five substituents, more preferably from one to three substituents, most preferably from one to two substituents, independently selected from the list of substituents. For example, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; pentafluorosulfanyl (i.e., SFs), sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like. The term “independently” when used in reference to substituents, means that when more than one of such substituents is possible, such substituents can be the same or different from each other.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

As used herein, Cm-Cn, such as C₁-C₁₁, C₁-C₈, or C₁-C₆ when used before a group refers to that group containing m to n carbon atoms.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Alkyl groups may be substituted or unsubstituted. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Cycloalkyl groups may be substituted or unsubstituted. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. In some embodiments, cycloalkylalkyl groups have from 4 to 16 carbon atoms, 4 to 12 carbon atoms, and typically 4 to 10 carbon atoms. Cycloalkylalkyl groups may be substituted or unsubstituted. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Alkenyl groups have from 2 to 12 carbon atoms, and typically from 2 to 10 carbons or, in some embodiments, from 2 to 8, 2 to 6, or 2 to 4 carbon atoms. In some embodiments, an alkenyl can have one carbon-carbon double bond, or multiple carbon-carbon double bonds, such as 2, 3, 4 or more carbon-carbon double bonds. Examples of alkenyl groups include, but are not limited to methenyl, ethenyl, propenyl, butenyl, etc. Alkenyl groups may be substituted or unsubstituted. Representative substituted alkenyl groups may be mono-substituted or substituted more than once, such as, but not limited to, mono-, di- or tri-substituted with substituents such as those listed above.

Cycloalkenyl groups include cycloalkyl groups as defined above, having at least one double bond between two carbon atoms. Cycloalkenyl group can be a mono- or polycyclic alkyl group having from 3 to 12, more preferably from 3 to 8 carbon atoms in the ring(s) and comprising at least one double bond between two carbon atoms. Cycloalkenyl groups may be substituted or unsubstituted. In some embodiments the cycloalkenyl group may have one, two or three double bonds or multiple carbon-carbon double bonds, such as 2, 3, 4, or more carbon-carbon double bonds. but does not include aromatic compounds. Cycloalkenyl groups have from 3 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, cyclobutadienyl, and cyclopentadienyl.

Cycloalkenylalkyl groups are alkyl groups as defined above in which a hydrogen or carbon bond of the alkyl group is replaced with a bond to a cycloalkenyl group as defined above. Cycloalkenylalkyl groups may be substituted or unsubstituted. Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

Alkynyl groups include straight and branched chain alkyl groups as defined above, except that at least one triple bond exists between two carbon atoms. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C═CH, —C═CCH₃, —CH₂C═CCH₃, —C═CCH₂CH(CH₂CH₃)₂, among others. Alkynyl groups may be substituted or unsubstituted. A terminal alkyne has at least one hydrogen atom bonded to a triply bonded carbon atom. Representative substituted alkynyl groups may be mono-substituted or substituted more than once, such as, but not limited to, mono-, di- or trisubstituted with substituents such as those listed above. A “cyclic alkyne” or “cycloalkynyl” is a cycloalkyl ring comprising at least one triple bond between two carbon atoms. Examples of cyclic alkynes or cycloalkynyl groups include, but are not limited to, cyclooctyne, bicyclononyne (BCN), difluorinated cyclooctyne (DIFO), dibenzocyclooctyne (DIBO), keto-DIBO, biarylazacyclooctynone (BARAC), dibenzoazacyclooctyne (DIBAC), dimethoxyazacyclooctyne (DIMAC), difluorobenzocyclooctyne (DIFBO), monobenzocyclooctyne (MOBO), and tetramethoxy DIBO (TMDIBO).

Aryl groups are cyclic aromatic hydrocarbons that do not contain heteroatoms. Aryl groups herein include monocyclic, bicyclic and tricyclic ring systems. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. Aryl groups may be substituted or unsubstituted. The phrase “aryl groups” includes groups containing fused rings, such as fused aromatic-aliphatic ring systems (e.g., indanyl, tetrahydronaphthyl, and the like). Representative substituted aryl groups may be monosubstituted or substituted more than once. For example, monosubstituted aryl groups include, but are not limited to, 2-, 3-, 4-, 5-, or 6-substituted phenyl or naphthyl groups, which may be substituted with substituents such as those listed above. Aryl moieties are well known and described, for example, in Lewis, R. J., ed., Hawley's Condensed Chemical Dictionary, 13^{th} Edition, John Wiley & Sons, Inc., New York (1997). An aryl group can be a single ring structure (i.e., monocyclic) or comprise multiple ring structures (i.e., polycyclic) that are fused ring structures. Preferably, an aryl group is a monocyclic aryl group.

Alkoxy groups are hydroxyl groups (—OH) in which the bond to the hydrogen atom is replaced by a bond to a carbon atom of a substituted or unsubstituted alkyl group as defined above. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Alkoxy groups may be substituted or unsubstituted. Representative substituted alkoxy groups may be substituted one or more times with substituents such as those listed above.

Similarly, alkylthio or thioalkoxy refers to an —SR group in which R is an alkyl attached to the parent molecule through a sulfur bridge, for example, —S-methyl, —S-ethyl, etc. Representative examples of alkylthio include, but are not limited to, —SCH₃, —SCH₂CH₃, etc.

The term “halogen” as used herein refers to bromine, chlorine, fluorine, or iodine. Correspondingly, the term “halo” means fluoro, chloro, bromo, or iodo. In some embodiments, the halogen is fluorine. In other embodiments, the halogen is chlorine or bromine.

The terms “hydroxy” and “hydroxyl” can be used interchangeably and refer to —OH.

The term “carboxy” refers to —COOH.

The term “cyano” refers to —CN.

The term “nitro” refers to —NO₂.

The term “isothiocyanate” refers to —N═C═S.

The term “isocyanate” refers to —N═C═O.

The term “azido” refers to —N₃.

The term “amino” refers to —NH₂. The term “alkylamino” refers to an amino group in which one or both of the hydrogen atoms attached to nitrogen is substituted with an alkyl group. An alkylamine group can be represented as —NR₂ in which each R is independently a hydrogen or alkyl group. For example, alkylamine includes methylamine (—NHCH₃), dimethylamine (—N(CH₃)₂), —NHCH₂CH₃, etc. The term “aminoalkyl” as used herein is intended to include both branched and straight-chain saturated aliphatic hydrocarbon groups substituted with one or more amino groups. Representative examples of aminoalkyl groups include, but are not limited to, —CH₂NH₂, —CH₂CH₂NH₂, and —CH₂CH(NH₂)CH₃.

As used herein, “amide” refers to —C(O)N(R)₂, wherein each R is independently an alkyl group or a hydrogen. Examples of amides include, but are not limited to, —C(O)NH₂, —C(O)NHCH₃, and —C(O)N(CH₃)₂.

The terms “hydroxylalkyl” and “hydroxyalkyl” are used interchangeably, and refer to an alkyl group substituted with one or more hydroxyl groups. The alkyl can be a branched or straight-chain aliphatic hydrocarbon. Examples of hydroxylalkyl include, but are not limited to, hydroxylmethyl (—CH₂OH), hydroxylethyl (—CH₂CH₂OH), etc.

As used herein, the term “heterocyclyl” includes stable monocyclic and polycyclic hydrocarbons that contain at least one heteroatom ring member, such as sulfur, oxygen, or nitrogen. As used herein, the term “heteroaryl” includes stable monocyclic and polycyclic aromatic hydrocarbons that contain at least one heteroatom ring member such as sulfur, oxygen, or nitrogen. Heteroaryl can be monocyclic or polycyclic, e.g., bicyclic or tricyclic. Each ring of a heterocyclyl or heteroaryl group containing a heteroatom can contain one or two oxygen or sulfur atoms and/or from one to four nitrogen atoms provided that the total number of heteroatoms in each ring is four or less and each ring has at least one carbon atom. Heteroaryl groups which are polycyclic, e.g., bicyclic or tricyclic must include at least one fully aromatic ring but the other fused ring or rings can be aromatic or non-aromatic. The heterocyclyl or heteroaryl group can be attached at any available nitrogen or carbon atom of any ring of the heterocyclyl or heteroaryl group. Preferably, the term “heteroaryl” refers to 5- or 6-membered monocyclic groups and 9- or 10-membered bicyclic groups which have at least one heteroatom (O, S, or N) in at least one of the rings, wherein the heteroatom-containing ring preferably has 1, 2, or 3 heteroatoms, more preferably 1 or 2 heteroatoms, selected from O, S, and/or N. The nitrogen heteroatom(s) of a heteroaryl can be substituted or unsubstituted. Additionally, the nitrogen and sulfur heteroatom(s) of a heteroaryl can optionally be oxidized (i.e., N→O and S(O)ᵣ, wherein r is 0, 1 or 2).

The term “ester” refers to —C(O)₂R, wherein R is alkyl.

The term “carbamate” refers to —OC(O)NR₂, wherein each R is independently alkyl or hydrogen.

The term “aldehyde” refers to —C(O)H.

The term “carbonate” refers to —OC(O)OR, wherein R is alkyl.

The term “maleimide” refers to a group with the chemical formula H₂C₂(CO)₂NH. The term “maleimido” refers to a maleimide group covalently linked to another group or molecule. Preferably, a maleimido group is N-linked, for example:

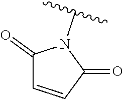 [SMILES: *N1C(=O)C=CC1=O]

The term “acyl halide” refers to —C(O)X, wherein X is halo (e.g., Br, Cl). Exemplary acyl halides include acyl chloride (—C(O)Cl) and acyl bromide (—C(O)Br).

In accordance with convention used in the art:

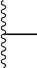

is used in structural formulas herein to depict the bond that is the point of attachment of the moiety, functional group, or substituent to the core, parent, or backbone structure, such as an antigen binding domain of the present invention.

When any variable occurs more than one time in any constituent or formula for a compound, its definition at each occurrence is independent of its definition at every other occurrence. Thus, for example, if a group is shown to be substituted with 0-3 R groups, then said group can be optionally substituted with up to three R groups, and at each occurrence, R is selected independently from the definition of R.

When a bond to a substituent is shown to cross a bond connecting two atoms in a ring, then such substituent can be bonded to any atom on the ring.

As used herein, the term “radiometal ion” or “radioactive metal ion” or “radioisotope” or “radiometal” refers to one or more isotopes of the elements that emit particles and/or photons. Any radiometal ion known to those skilled in the art in view of the present disclosure can be used in the invention. Non-limiting examples of radioisotopes that may be used for therapeutic applications in accordance with the present invention include, e.g., beta or alpha emitters, such as, e.g., ²²⁵Ac, ¹⁷⁷Lu, ³²P, ⁴⁷Sc, ⁶⁷Cu, ⁷⁷As, ⁸⁹Sr, ⁹⁰Y ⁹⁹Tc, ¹⁰⁵Rh, ¹⁰⁹Pd, ¹¹¹Ag, ¹³¹I, ¹⁴⁹Tb, ⁵²Tb, ¹⁵⁵Tb, ¹⁵³Sm, ¹⁵⁹Gd, ¹⁶⁵Dy, ¹⁶⁶Ho, ¹⁶⁹Er, ¹⁸⁶Re, ¹⁸⁸Re, ¹⁹⁴Ir, ¹⁹⁸Au, ¹⁹⁹Au, ²¹¹At, ²¹²Pb, ²¹²Bi, ²¹³Bi, ²²³Ra ²⁵⁵Fm and ²²⁷Th. Other non-limiting examples of radioisotopes that may be used as imaging agents in accordance with the present invention include gamma-emitting radioisotopes, such as, e.g., ¹⁷⁷Lu, ⁶²CU, ⁶⁴Cu, ⁶⁷Ga, ⁶⁸Ga, ⁸⁶Y, ⁸⁹Zr, and ¹¹¹In. In certain embodiments, the radiometal ion is a “therapeutic emitter,” meaning a radiometal ion that is useful as a therapeutic agent, e.g., as a cytotoxic agent that is capable of reducing or inhibiting the growth of, or in particular killing, a cancer cell, such as a prostate cancer cell. Examples of therapeutic emitters include, but are not limited to, beta or alpha emitters, such as, ¹³²La, ¹³⁵La, ¹³⁴Ce, ¹⁴⁴Nd, ¹⁴⁹Tb, ¹⁵²Tb, ¹⁵⁵Tb, ¹⁵³Sm, ¹⁵⁹Gd, ¹⁶⁵Dy, ¹⁶⁶Ho, ¹⁶⁹Er, ¹⁷⁷Lu, ¹⁸⁶Re, ¹⁸⁸Re, ⁹⁴Ir, ¹⁹⁸Au, ¹⁹⁹Au, ²¹¹At, ²¹²Pb, ²¹²Bi, ²¹³Bi, ²²³Ra, ²²⁵Ac, ²⁵⁵Fm and ²²⁷Th, ²²⁶Th, ²³⁰U. Preferably, a radiometal ion used in the invention is an alpha-emitting radiometal ion, such as actinium-225 (²²⁵Ac).

A “radiometal complex” as used herein refers to a complex comprising a radiometal ion associated with a chelator that is a macrocyclic compound. Typically, a radiometal ion is bound to or coordinated to a macrocyclic compound via coordinate bonding. Heteroatoms of the macrocyclic ring can participate in coordinate bonding of a radiometal ion to a macrocycle compound. A macrocycle compound can be substituted with one or more substituent groups, and the one or more substituent groups can also participate in coordinate bonding of a radiometal ion to a macrocycle compound in addition to, or alternatively to the heteroatoms of the macrocyclic ring.

### Immunoconjugates

Embodiments of the present invention relate to compositions and methods for targeting hK2 with a short half-life Fab-based radioconjugate to achieve efficacious tumor cell death in prostate cancer patients; preferably, such radioconjugates comprise a Fab (instead of a full-length antibody) and demonstrate an improved safety profile (e.g., as measured by bone marrow toxicity) compared to full-length antibody-based radioconjugates. Embodiments of the Fab-based radioconjugates of the present invention target hK2-expressing prostate cancer cells and demonstrate a short half-life.

As used herein, an “immunoconjugate” refers to an antibody, or an antigen binding domain, that is conjugated (joined, e.g., bound via a covalent bond) to a second molecule, such as a toxin, drug, radiometal ion, chelator, radiometal complex, etc. A “radioimmunoconjugate” (also referred to herein as a “radioconjugate”) in particular is an immunoconjugate in which an antibody or antigen binding domain is labeled with a radiometal or conjugated to a radiometal complex.

According to embodiments of the present invention, an immunoconjugate comprises a therapeutic moiety conjugated to an antigen binding domain of the present invention that has binding specificity for hK2. As used herein, a “therapeutic moiety” that forms part of an immunoconjugate may be useful in therapeutic applications and/or imaging applications, i.e., as a therapeutic agent (e.g., a cytotoxic agent) and/or an imaging agent, respectively. For example, therapeutic moieties of the present invention may comprise radiometals. It is noted that certain radiometals may be used as therapeutic agents (e.g., ²²⁵Ac) and/or as imaging agents (e.g., ¹¹¹In). A suitable therapeutic agent is one that is capable of reducing or inhibiting the growth of, or in particular killing, a cancer cell, such as a prostate cancer cell. In certain embodiments, radioconjugates comprising a radioisotope conjugated to an antigen binding domain can deliver a cytotoxic payload with the ability to emit alpha and/or beta particles in the vicinity of a tumor by binding onto cancer cells' surface antigens and initiating cell death.

According to certain embodiments, an immunoconjugate comprising an imaging agent, such as Cu-64, may be used to detect prostate cancer cells in a subject. According to an embodiment, a method of detecting the presence of prostate cancer in a subject comprises administering an immunoconjugate of the present invention to a subject suspected to have prostate cancer and visualizing biological structures to which the conjugate is bound (e.g., by computerized tomography or positron emission tomography), thereby detecting the presence of prostate cancer. According to another embodiment, a method of detecting the progress of cancer treatment in a subject (e.g., after the subject has begun treatment for prostate cancer) comprises administering an immunoconjugate of the present invention to the subject and visualizing biological structures to which the immunoconjugate is bound (e.g., by computerized tomography or positron emission tomography), thereby detecting the progress of prostate cancer treatment in the subject (e.g., detecting whether prostate cancer cells have reduced following the prostate cancer treatment). According an embodiment, the method may further comprise administering an anti-cancer therapeutic (e.g., an anti-hK2 therapeutic that targets hK2-expressing cancer cells) to the subject if prostate cancer has been detected in the subject.

According to preferred embodiments, the present invention relates to short half-life immunoconjugates (e.g. short half-life radioimmunococonjugates). As used herein, a “short half-life immunoconjugate” refers to an immunoconjugate comprising an antigen binding domain (e.g., a Fab), wherein the immunoconjugate has an in vivo half-life that is shorter than the in vivo half-life of a comparator immunoconjugate, wherein the comparator immunoconjugate is identical to the short half-life immunoconjugate except the antigen binding domain of the comparator immunoconjugate is replaced with a full-length antibody comprising the antigen binding domain (e.g., a full-length IgG comprising an Fc region and the antigen binding domain). In certain embodiments, a short half-life immunoconjugate of the present invention has a half-life of 36 hours or less, or 24 hours or less, or 12 hours or less, or 6 hours or less, or 3 hours or less. For example, a short half-life immunoconjugate of the present invention may have a half-life from about 1 hour to about 36 hours, or from about 1 hour to about 24 hours, or from about 1 hour to about 12 hours, or from about 1 hour to about 6 hours, or from about 1 hour to about 3 hours, or from about 2 hours to about 3 hours.

Actinium-225 (²²⁵Ac) is an alpha-emitting radioisotope that is of particular interest for medical applications. Another radioisotope of interest for medical applications is Lutetium-177 (¹⁷⁷Lu), which emits both gamma-irradiation suitable for imaging and medium-energy beta-irradiation suitable for radiotherapy. Non-limiting examples of radioisotopes that may be used for therapeutic applications in accordance with the present invention include, e.g., beta or alpha emitters, such as, e.g., ²²⁵Ac, ¹⁷⁷Lu, ³²P, ⁴⁷Sc, ⁶⁷Cu, ⁷⁷As, ⁸⁹Sr, ⁹⁰Y ⁹⁹Tc, ¹⁰⁵Rh, ¹⁰⁹Pd, ¹¹¹Ag, ¹³¹I, ¹⁴⁹Tb, ⁵²Tb, ¹⁵⁵Tb, ¹⁵³Sm, ¹⁵⁹Gd, ¹⁶⁵Dy, ¹⁶⁶Ho, ¹⁶⁹Er, ¹⁸⁶Re, ¹⁸⁸Re, ¹⁹⁴Ir, ¹⁹⁸Au, ¹⁹⁹Au, ²¹¹At, ²¹²Pb, ²¹²Bi, ²¹³Bi, ²²³Ra, ²⁵⁵Fm and ²²⁷Th. Other non-limiting examples of radioisotopes that may be used as imaging agents in accordance with the present invention include gamma-emitting radioisotopes, such as, e.g., ¹⁷⁷Lu, ⁶²Cu, ⁶⁴Cu, ⁶⁷Ga, ⁶⁸Ga, ⁸⁶Y, ⁸⁹Zr, and ¹¹¹In.

In certain embodiments, the therapeutic moiety is a cytotoxic agent that is an auristatin derivative, such as MMAE (monomethyl auristatin E) or MMAF (monomethyl auristatin F). For example, the auristatin derivative may be attached to the antibody or antigen binding domain of the invention through the N (amino) terminus or the C (carboxyl) terminus of the peptidic drug moiety (WO02/088172), or via any cysteine engineered into the antibody or antigen binding domain.

### Anti-hK2 Antibodies and Antigen Binding Domains

As described herein, embodiments of the present invention relate to an immunoconjugate comprising a therapeutic moiety conjugated to an antigen binding domain with binding specificity for kallikrein related peptidase 2 (hK2). According to certain embodiments, the antigen binding domain is a scFv, a (scFv)₂, a Fv, a Fab, a F(ab′)₂, a Fd, a dAb or a VHH. According to a preferred embodiment, the antigen binding domain with binding specificity for hK2 is a Fab.

According to certain embodiments, the antigen binding domain that binds hK2 (e.g., a Fab) comprises the HCDR1, the HCDR2, the HCDR3, the LCDR1, the LCDR2 and the LCDR3 of SEQ ID NOs: 170, 171, 172, 173, 174 and 175, respectively.

According to certain embodiments, the antigen binding domain that binds hK2 (e.g., a Fab) comprises a VH which is at least 80% (e.g. at least 85%, at least 90%, at least 95%, at least 99% or 100%) identical to the VH of SEQ ID NO: 162 and a VL which is at least 80% (e.g. at least 85%, at least 90%, at least 95%, at least 99% or 100%) identical to the VL of SEQ ID NO: 163. For example, the antigen binding domain that binds hK2 (e.g., a Fab) comprises a VH which is at least 95% identical to the VH of SEQ ID NO: 162 and a VL which is at least 95% identical to the VL of SEQ ID NO: 163. According to certain embodiments, the antigen binding domain that binds hK2 (e.g., a Fab) comprises the VH of SEQ ID NO: 162 and the VL of SEQ ID NO: 163.

According to certain embodiments, the antigen binding domain is a “KL2B30 Fab,” also referred to as “Fab of KL2B30,” which is a Fab that comprises (a) a HCDR1, a HCDR2, a HCDR3, a LCDR1, a LCDR2 and a LCDR3 of SEQ ID NOs: 170, 171, 172, 173, 174 and 175, respectively; and/or (b) a VH which is at least 95% identical, or 100% identical, to SEQ ID NO: 162 and a VL which is at least 95% identical, or 100% identical, to SEQ ID NO: 163.

Non-limiting examples of KL2B30 Fabs include KL2B997 and KL2B1251, which are described in the example section below. The heavy chain and light chain sequences of KL2B997 (SEQ ID NO: 472 and SEQ ID NO: 473, respectively) and KL2B1251 (SEQ ID NO: 474 and SEQ ID NO: 475, respectively) are provided in FIG. 2. KL2B997 has a His-tag and a sortase tag (underlined in FIG. 2) on the heavy chain, and KL2B1251 is tagless.

According to certain embodiments, the antigen binding domain that binds hK2 comprises a heavy chain which is at least 80% (e.g. at least 85%, at least 90%, at least 95%, at least 99% or 100%) identical to SEQ ID NO: 474 and a light chain which is at least 80% (e.g. at least 85%, at least 90%, at least 95%, at least 99% or 100%) identical to SEQ ID NO: 475. In an embodiment, the antigen binding domain is a Fab comprising a heavy chain which is at least 95%, at least 99% or 100% identical to SEQ ID NO: 474 and a light chain which is at least 95%, at least 99% or 100% identical to SEQ ID NO: 475.

According to certain embodiments, the antigen binding domain that binds hK2 comprises a heavy chain which is at least 80% (e.g. at least 85%, at least 90%, at least 95%, at least 99% or 100%) identical to SEQ ID NO: 472 and a light chain which is at least 80% (e.g. at least 85%, at least 90%, at least 95%, at least 99% or 100%) identical to SEQ ID NO: 473. In an embodiment, the antigen binding domain is a Fab comprising a heavy chain which is at least 95%, at least 99% or 100% identical to SEQ ID NO: 472 and a light chain which is at least 95%, at least 99% or 100% identical to SEQ ID NO: 473.

In some embodiments, an immunoconjugate of the present invention comprises an antigen binding domain that comprises VL, VH or CDRs having amino acid sequences of certain antibodies described below, selected from the group consisting of m11B6, hu11B6, HCF3-LCD6, HCG5-LCB7, KL2B357, KL2B358, KL2B359, KL2B360, KL2B413, KL2B30, KL2B53, KL2B242, KL2B467 and KL2B494. The foregoing antibodies and antigen binding domains, and methods of making them, are described in PCT/IB2020/056972, which is incorporated by reference herein.

An embodiment of the present invention provides a radioimmunoconjugate having the following structure (which does not show the lysine residue of the Fab that is linked to the phenylthiourea moiety):

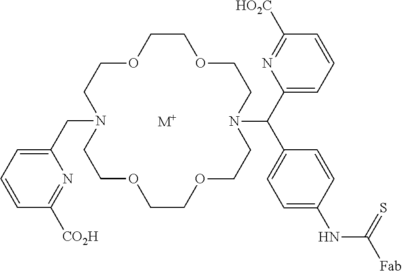

(also referred to as TOPA-[C7]-phenylthiourea-Fab),

wherein M⁺ is a radioisotope, such as actinium-225(²⁵Ac), and

wherein the Fab has binding specificity for hK2, such as a KL2B30 Fab (e.g., KL2B997 or KL2B1251). The Fab preferably comprises (a) a HCDR1, a HCDR2, a HCDR3, a LCDR1, a LCDR2 and a LCDR3 of SEQ ID NOs: 170, 171, 172, 173, 174 and 175, respectively; and/or (b) a VH which is at least 95% identical, or 100% identical, to SEQ ID NO: 162 and a VL which is at least 95% identical, or 100% identical, to SEQ ID NO: 163.

An embodiment of the present invention provides a radioimmunoconjugate having the following structure (which does not show the lysine residue of the Fab that is linked to the phenylthiourea moiety):

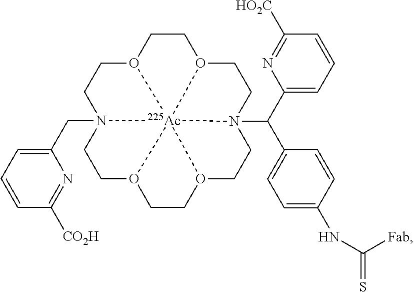

wherein the Fab has binding specificity for hK2, such as a KL2B30 Fab (e.g., KL2B997 or KL2B1251). The Fab preferably comprises (a) a HCDR1, a HCDR2, a HCDR3, a LCDR1, a LCDR2 and a LCDR3 of SEQ ID NOs: 170, 171, 172, 173, 174 and 175, respectively; and/or (b) a VH which is at least 95% identical, or 100% identical, to SEQ ID NO: 162 and a VL which is at least 95% identical, or 100% identical, to SEQ ID NO: 163.

An embodiment of the present invention provides a Fab conjugated to NOTA or a derivative of NOTA, such as NODA or NODA-GA. An embodiment of the present invention provides a NODA-GA-Fab and may be represented by the structure in FIG. 4 (which does not show any lysine chains of the Fab linked to the chelator-linker), wherein M⁺ is a radioisotope, such as Cu-64 (⁶⁴Cu), and wherein the Fab has binding specificity for hK2, such as a KL2B30 Fab (e.g., KL2B997 or KL2B1251). The Fab preferably comprises (a) a HCDR1, a HCDR2, a HCDR3, a LCDR1, a LCDR2 and a LCDR3 of SEQ ID NOs: 170, 171, 172, 173, 174 and 175, respectively; and/or (b) a VH which is at least 95% identical, or 100% identical, to SEQ ID NO: 162 and a VL which is at least 95% identical, or 100% identical, to SEQ ID NO: 163.

An embodiment of the present invention provides a NOD A-GA-Fab as represented by the structure in FIG. 4 (which does not show any lysine chains of the Fab linked to the chelator-linker), wherein M⁺ is Cu-64 (⁶⁴Cu), and wherein the Fab is a KL2B30 Fab (e.g., KL2B997 or KL2B1251) which comprises (a) a HCDR1, a HCDR2, a HCDR3, a LCDR1, a LCDR2 and a LCDR3 of SEQ ID NOs: 170, 171, 172, 173, 174 and 175, respectively; and/or (b) a VH which is at least 95% identical, or 100% identical, to SEQ ID NO: 162 and a VL which is at least 95% identical, or 100% identical, to SEQ ID NO: 163. A method of detecting hK2-expressing prostate cancer cells in a subject comprises administering the NODA-GA-KL2B30 Fab to a subject suspected to have prostate cancer and visualizing biological structures to which the NODA-GA-KL2B30 Fab is bound (preferably by positron emission tomography), thereby detecting the hK2-expressing prostate cancer cells.

### SPECIFIC ENUMERATED EMBODIMENTS

Exemplary enumerated embodiments of the present invention are provided below.
1. An immunoconjugate comprising: a therapeutic moiety conjugated to an antigen binding domain with binding specificity for kallikrein related peptidase 2 (hK2).
2. The immunoconjugate of embodiment 1, wherein the therapeutic moiety is a cytotoxic agent.
3. The immunoconjugate of embodiment 1, wherein the therapeutic moiety is an imaging agent.
4. The immunoconjugate of any of embodiments 1-3, wherein the therapeutic moiety comprises a radiometal.
5. The immunoconjugate of embodiment 4, wherein the radiometal is selected from the group consisting of ²²⁵Ac, ¹⁷⁷Lu, ³²P, ⁴⁷Sc, ⁶⁷Cu, ⁷⁷As, ⁸⁹Sr, ⁹⁰Y ⁹⁹Tc, ¹⁰⁵Rh, ¹⁰⁹Pd, ¹¹¹Ag, ¹³¹I, ¹⁴⁹Tb, ⁵²Tb, ¹⁵⁵Tb, ¹⁵³Sm, ¹⁵⁹Gd, ¹⁶⁵Dy, ¹⁶⁶Ho, ¹⁶⁹Er, ¹⁸⁶Re, ¹⁸⁸Re, ¹⁹⁴Ir, ¹⁹⁸Au, ¹⁹⁹Au, ²¹¹At, ²¹²Pb, ²¹²Bi, ²¹³Bi, ²²³Ra, ²⁵⁵Fm, ²²⁷Th, ¹⁷⁷Lu, ⁶²Cu, ⁶⁴Cu, ⁶⁷Ga ⁶⁸Ga, 8⁶Y, ⁸⁹Zr, and ¹¹¹In.
6. The immunoconjugate of embodiment 1, wherein the therapeutic moiety is a cytotoxic agent comprising ²²⁵Ac.
7. The immunoconjugate of embodiment 1, wherein the therapeutic moiety is an imaging agent comprising ¹¹¹In or ⁶⁴Cu.
8. The immunoconjugate according to any of embodiments 4-7, wherein the therapeutic moiety comprises a radiometal complex, wherein the radiometal complex comprises the radiometal bound to a chelator, and wherein the chelator is conjugated to the antigen binding domain with binding specificity for kallikrein related peptidase 2 (hK2).
9. The immunoconjugate according to embodiment 8, wherein the chelator is 1,4,7,10-tetraazacyclododecane-1,4,7,10,tetraacetic acid (DOTA), S-2-(4-isothiocyanatobenzyl)-1,4,7-triazacyclononane-1,4,7-triacetic acid (NOTA), 1,4,8,11-tetraazacyclodocedan-1,4,8,11-tetraacetic acid (TETA), 3,6,9,15-tetraazabicyclo[9.3.1]-pentadeca-1(15),11,13-triene-4-(S)-(4-isothiocyanatobenzyl)-3,6,9-triacetic acid (PCTA), 5-S-(4-aminobenzyl)-1-oxa-4,7,10-triazacyclododecane-4,7,10-tris(acetic acid) (DO3A), or a derivative thereof.
10. The immunoconjugate according to embodiment 8, wherein the chelator is DOTA or NOTA or a derivative thereof.
11. The immunoconjugate according to embodiment 8, wherein the chelator is H₂bp18c6 or a H₂bp18c6 derivative.
12. The immunoconjugate according to embodiment 8, wherein the radiometal complex is a radiocomplex of Formula (I-m), or Formula (II-m), or Formula (III-m) as described herein, wherein R₁₁ comprises the antigen binding domain with binding specificity for kallikrein related peptidase 2 (hK2) and M is the radiometal.
13. The immunoconjugate according to embodiment 8, wherein the radiometal complex is a radiometal complex of Formula (IV-m), or Formula (V-m), or Formula (VI-m) as described herein, wherein R₄ comprises the antigen binding domain with binding specificity for kallikrein related peptidase 2 (hK2) and M+ is the radiometal.
14. The immunoconjugate of embodiment 1, wherein the therapeutic moiety is an auristatin derivative.
15. The immunoconjugate of embodiment 14, wherein the therapeutic moiety is MMAE (monomethyl auristatin E).
16. The immunoconjugate of embodiment 14, wherein the therapeutic moiety is MMAF (monomethyl auristatin F).
17. The immunoconjugate according to any of embodiments 1-16 or 51-56, wherein the antigen binding domain that binds hK2 is a scFv, a (scFv)₂, a Fv, a Fab, a F(ab′)₂, a Fd, a dAb or a VHH.
18. The immunoconjugate according to any of embodiments 1-16 or 51-56, wherein the antigen binding domain with binding specificity for hK2 is a Fab.
19. The immunoconjugate according to any of embodiments 1-18 or 51-56, wherein the antigen binding domain comprises the HCDR1, the HCDR2, the HCDR3, the LCDR1, the LCDR2 and the LCDR3 of SEQ ID NO: 170 (SYYWS), SEQ ID NO: 171 (YIYYSGSTNYNPSLKS), SEQ ID NO: 172 (TTIFGVVTPNFYYGMDV), SEQ ID NO: 173 (RASQGISSYLA), SEQ ID NO: 174 (AASTLQS) and SEQ ID NO: 175 (QQLNSYPLT), respectively.
20. The immunoconjugate according to any of embodiments 1-19 or 51-56, wherein the antigen binding domain that binds hK2 comprises a VH which is at least 80% (e.g. at least 85%, at least 90%, at least 95%, at least 99% or 100%) identical to the VH of SEQ ID NO: 162 (QVQLQESGPGLVKPSETLSLTCTVSGGSISSYYWSWIRQPPGKGLEWIGYIYYSGSTNYNPSL KSRVTISVDTSKNQFSLKLSSVTAADTAVYYCAGTTIFGVVTPNFYYGMDVWGQGTTVTVS S), and a VL which is at least 80% (e.g. at least 85%, at least 90%, at least 95%, at least 99% or 100%) identical to the VL of SEQ ID NO: 163 (DIQMTQSPSFLSASVGDRVTITCRASQGISSYLAWYQQKPGKAPKFLIYAASTLQSGVPSRFS GSGSGTEFTLTISSLQPEDFATYYCQQLNSYPLTFGGGTKVEIK).
21. The immunoconjugate according to any of embodiments 1-19 or 51-56, wherein the antigen binding domain that binds hK2 comprises the VH of SEQ ID NO: 162 and the VL of SEQ ID NO: 163.
22. The immunoconjugate according to any of embodiments 1-18 or 51-56, wherein the antigen binding domain is a Fab that comprises:
   a. a HCDR1, a HCDR2, a HCDR3, a LCDR1, a LCDR2 and a LCDR3 of SEQ ID NOs: 170, 171, 172, 173, 174 and 175, respectively; and/or
   b. a VH of SEQ ID NO: 162 and a VL of SEQ ID NO: 163; and/or
   c. a heavy chain which is at least 95%, at least 99% or 100% identical to SEQ ID NO: 474 and a light chain which is at least 95%, at least 99% or 100% identical to SEQ ID NO: 475.
23. The immunoconjugate according to any of embodiments 1-22 or 51-56, wherein the immunoconjugate is a short half-life immunoconjugate.
24. A method of treating an hK2-expressing cancer in a subject, comprising administering to the subject a therapeutically effective amount of the immunoconjugate according to any of embodiments 1-23 or 51-56.
25. A method of reducing the amount of hK2-expressing tumor cells in a subject, comprising administering to the subject a therapeutically effective amount of the immunoconjugate according to any of embodiments 1-23 or 51-56.
26. A method of treating prostate cancer in a subject, comprising administering to the subject a therapeutically effective amount of the immunoconjugate according to any of embodiments 1-23 or 51-56.
27. The method of embodiment 26, wherein the prostate cancer is relapsed, refractory, malignant or castration resistant prostate cancer, or any combination thereof.
28. The method of embodiment 26, wherein the prostate cancer is metastatic castration-resistant prostate cancer.
29. A method of detecting the presence of prostate cancer in a subject, comprising administering the immunoconjugate according to any of embodiments 1-23 or 51-56 to a subject suspected to have prostate cancer and visualizing the biological structures to which the conjugate is bound (e.g., by computerized tomography or positron emission tomography), thereby detecting the presence of prostate cancer, wherein the immunoconjugate preferably comprises an imaging agent, such as 111-In or 64-Cu.
30. A method of making an immunoconjugate according to any of embodiments 1-23 or 51-56 comprising: conjugating the therapeutic moiety to the antigen binding domain with binding specificity for kallikrein related peptidase 2 (hK2).
31. A method of making a radioimmunoconjugate comprising binding a radiometal to a chelator that is conjugated to an antigen binding domain with binding specificity for kallikrein related peptidase 2 (hK2).
32. The method of embodiment 31, wherein the chelator is DOTA or NOTA or a derivative thereof.
33. The method of embodiment 31, wherein the chelator is H₂bp18c6 or a H₂bp18c6 derivative.
34. The method of embodiment 31, wherein the chelator is selected from the group consisting of chelators of Formula (I), Formula (II) and Formula (III) as described herein, wherein Rn comprises the antigen binding domain with binding specificity for kallikrein related peptidase 2 (hK2).
35. The method of embodiment 31, wherein the chelator is selected from the group consisting of chelators of Formula (IV), Formula (V) and Formula (VI) as described herein, wherein R₄ comprises the antigen binding domain with binding specificity for kallikrein related peptidase 2 (hK2).
36. The method according to any of embodiments 31-35, wherein the antigen binding domain that binds hK2 is a scFv, a (scFv)₂, a Fv, a Fab, a F(ab′)₂, a Fd, a dAb or a VHH.
37. The method according to any of embodiments 31-35, wherein the antigen binding domain that binds hK2 is a Fab.
38. The method according to any of embodiments 31-37, wherein the antigen binding domain comprises the HCDR1, the HCDR2, the HCDR3, the LCDR1, the LCDR2 and the LCDR3 of SEQ ID NOs: 170, 171, 172, 173, 174 and 175, respectively.
39. The method according to any of embodiments 31-38, wherein the antigen binding domain that binds hK2 comprises a VH which is at least 80% (e.g. at least 85%, at least 90%, at least 95%, at least 99% or 100%) identical to the VH of SEQ ID NO: 162 and a VL which is at least 80% (e.g. at least 85%, at least 90%, at least 95%, at least 99% or 100%) identical to the VL of SEQ ID NO: 163.
40. The method according to any of embodiments 31-38, wherein the antigen binding domain that binds hK2 comprises the VH of SEQ ID NO: 162 and the VL of SEQ ID NO: 163.
41. The method according to any of embodiments 31-37, wherein the antigen binding domain is a Fab that comprises:
   a. a HCDR1, a HCDR2, a HCDR3, a LCDR1, a LCDR2 and a LCDR3 of SEQ ID NOs: 170, 171, 172, 173, 174 and 175, respectively; and/or
   b. a VH of SEQ ID NO: 162 and a VL of SEQ ID NO: 163.
42. A short half-life radioimmunoconjugate comprising a radiometal complex, wherein the radiometal complex comprises ¹¹⁵Ac bound to a chelator, and wherein the chelator is conjugated to a Fab with binding specificity for hK2, said Fab comprising: a HCDR1, a HCDR2, a HCDR3, a LCDR1, a LCDR2 and a LCDR3 of SEQ ID NOs: 170, 171, 172, 173, 174 and 175, respectively.
43. The short half-life radioimmunoconjugate of embodiment 42, said Fab comprising a VH of SEQ ID NO: 162 and a VL of SEQ ID NO: 163.
44. The short half-life radioimmunoconjugate of embodiment 42 or embodiment 43, wherein the chelator is DOTA or NOTA or a derivative thereof.
45. The short half-life radioimmunoconjugate of embodiment 42 or embodiment 43, wherein the chelator is H₂bp18c6 or a H₂bp18c6 derivative.
46. The short half-life radioimmunoconjugate of embodiment 42 or embodiment 43, wherein the radiometal complex is selected from the group consisting of chelators of Formula (I-m), Formula (II-m) and Formula (III-m) as described herein, wherein R₁₁ comprises the Fab with binding specificity for hK2.
47. The short half-life radioimmunoconjugate of embodiment 42 or embodiment 43, wherein the radiometal complex is selected from the group consisting of chelators of Formula (IV-m), Formula (V-m) and Formula (VI-m) as described herein, wherein R₄ comprises the Fab with binding specificity for hK2.
48. The method according to any of embodiments 31-37 further comprising conjugating the chelator to the antigen binding domain with binding specificity for kallikrein related peptidase 2 (hK2) prior to binding the radiometal to the chelator.
49. The method according to embodiment 48, wherein the chelator has the structure of

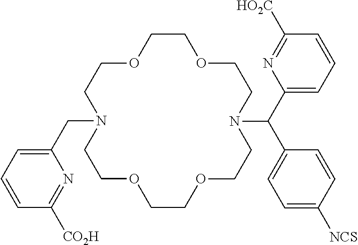

prior to conjugating the chelator to the antigen binding domain.
50. The method according to embodiment 48, wherein the chelator has the structure of

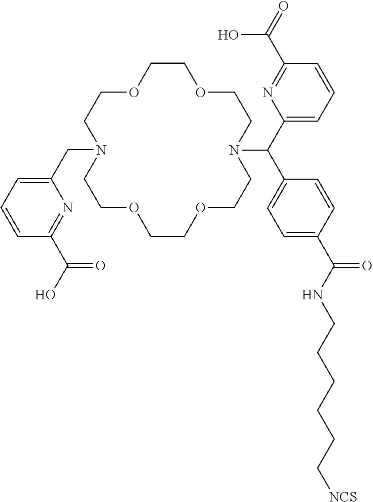

prior to conjugating the chelator to the antigen binding domain.
51. The immunoconjugate according to embodiment 8, wherein the radiometal complex is a radiometal complex of Formula (I-m),
   wherein the radiometal complex of Formula (I-m) has the following structure:

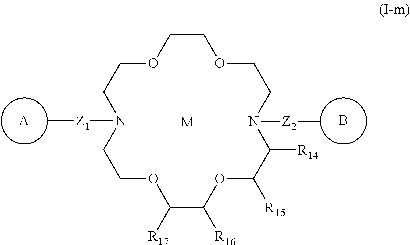

wherein:
   M is the radiometal, preferably an alpha-emitting radiometal ion, and more preferably actinium-225 (²²⁵Ac);
   each of ring A and ring B is independently a 6-10 membered aryl or a 5-10 membered heteroaryl, wherein each of ring A and ring B is optionally substituted with one or more substituents independently selected from the group consisting of halo, alkyl, alkenyl, cycloalkyl, cycloalkenyl, aryl, heterocyclyl, heteroaryl, —OR₁₃, —SR₁₃, —(CH₂)ₚCOOR₁₃, —OC(O)R₁₃, —N(R₁₃)₂, —CON(R₁₃)₂, —NO₂, —CN —OC(O)N(R₁₃)₂, and X; each of Z₁ and Z₂ is independently —(C(R₁₂)₂)ₘ— or —(CH₂)ₙ—C(R₁₂)(X)—(CH₂)ₙ—;
each X is independently -L₁-R₁₁;
each n is independently 0, 1, 2, 3, 4, or 5;
each m is independently 1, 2, 3, 4, or 5;
each p is independently 0 or 1;
L₁ is absent or a linker;
R₁₁ comprises the antigen binding domain with binding specificity for kallikrein related peptidase 2 (hK2);
each R₁₂ is independently hydrogen, alkyl, cycloalkyl, aryl, heterocyclyl, or heteroaryl;
each R₁₃ is independently hydrogen or alkyl;
each of R₁₄, R₁₅, R₁₆, and R₁₇ is independently hydrogen, alkyl, or X,
or alternatively R₁₄ and R₁₅ and/or R₁₆ and R₁₇ are taken together with the carbon atoms to which they are attached to form a 5- or 6-membered cycloalkyl ring optionally substituted with X; provided that the radiometal complex comprises at least one X, and when X is present on ring A or ring B, L₁ is a linker or at least one of R₁₂ and R₁₄-R₁₇ is not hydrogen.
52. The immunoconjugate according to embodiment 8, wherein the radiometal complex is a radiometal complex of Formula (II-m),
   wherein the radiometal complex of Formula (II-m) has the following structure:

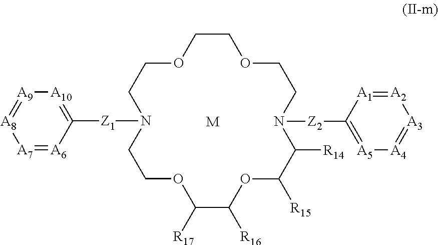

wherein:
M is the radiometal, preferably an alpha-emitting radiometal ion, and more preferably actinium-225 (²²⁵Ac);
A₁ is N or CR₁ or is absent;
A₂ is N or CR₂;
A₃ is N or CR₃;
A₄ is N or CR₄;
A₅ is N or CR₅;
A₆ is N or CR₆ or is absent;
A₇ is N or CR₇;
A₈ is N or CR₈;
A₉ is N or CR₉;
A₁₀ is N or CR₁₀;
provided that no more than three of A₁, A₂, A₃, A₄, and A₅ are N, and no more than three of A₆, A₇, A₈, A₉, and A₁₀ are N;
each of R₁, R₂, R₃, R₄, R₅, R₆, R₇, R₈, R₉, and R₁₀ is independently selected from the group consisting of hydrogen, halo, alkyl, alkenyl, cycloalkyl, cycloalkenyl, aryl, heterocyclyl, heteroaryl, —OR₁₃, —SR₁₃, —(CH₂)ₚCOOR₁₃, —OC(O)R₁₃, —N(R₁₃)₂, —CON(R₁₃)₂, —NO₂, —CN —OC(O)N(R₁₃)₂, and —X,
or, alternatively, any two directly adjacent R₁, R₂, R₃, R₄, R₅, R₆, R₇, R₈, R₉, and R₁₀ are taken together with the atoms to which they are attached to form a five or six-membered substituted or unsubstituted carbocyclic or nitrogen-containing ring;
each of Z₁ and Z₂ is independently —(C(R₁₂)₂)ₘ— or —(CH₂)ₙ—C(R₁₂)(X)—(CH₂)ₙ—;
each X is independently -L₁-R₁₁;
each n is independently 0, 1, 2, 3, 4, or 5;
each m is independently 1, 2, 3, 4, or 5;
each p is independently 0 or 1;
L₁ is absent or a linker;
R₁₁ comprises the antigen binding domain with binding specificity for kallikrein related peptidase 2 (hK2);
each R₁₂ is independently hydrogen, alkyl, cycloalkyl, aryl, heterocyclyl, or heteroaryl;
each R₁₃ is independently hydrogen or alkyl;
each of R₁₄, R₁₅, R₁₆, and R₁₇ is independently hydrogen, alkyl, or X,
or alternatively R₁₄ and R₁₅ and/or R₁₆ and R₁₇ are taken together with the carbon atoms to which they are attached to form a 5- or 6-membered cycloalkyl ring optionally substituted with X;
provided that the radiometal complex comprises at least one X, and when any one of R₁, R₂, R₃, R₄, R₅, R₆, R₇, R₈, R₉, and R₁₀ is X, then L₁ is a linker or at least one of R₁₂ and R₁₄-R₁₇ is not hydrogen.
53. The immunoconjugate according to embodiment 8, wherein the radiometal complex is a radiometal complex of Formula (III-m),
   wherein the radiometal complex of Formula (III-m) has the following structure:

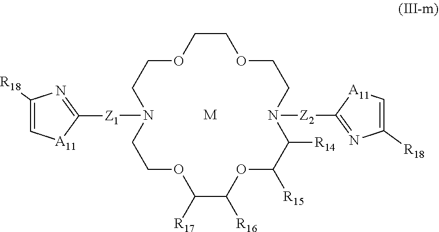

wherein:
M is the radiometal, preferably an alpha-emitting radiometal ion, and more preferably actinium-225 (²²⁵Ac);
each A₁₁ is independently O, S, NMe, or NH;
each of Z₁ and Z₂ is independently —(C(R₁₂)₂)ₘ— or —(CH₂)ₙ—C(R₁₂)(X)—(CH₂)ₙ—;
each X is independently -L₁-R₁₁;
each n is independently 0, 1, 2, 3, 4, or 5;
each m is independently 1, 2, 3, 4, or 5;
each p is independently 0 or 1;
L₁ is absent or a linker;
R₁₁ comprises the antigen binding domain with binding specificity for kallikrein related peptidase 2 (hK2);
each R₁₂ is independently hydrogen, alkyl, cycloalkyl, aryl, heterocyclyl, or heteroaryl;
each R₁₃ is independently hydrogen or alkyl;
each of R₁₄, R₁₅, R₁₆, and R₁₇ is independently hydrogen, alkyl, or X,
or alternatively R₁₄ and R₁₅ and/or R₁₆ and R₁₇ are taken together with the carbon atoms to which they are attached to form a 5- or 6-membered cycloalkyl ring optionally substituted with X; and
each R₁₈ is independently selected from the group consisting of hydrogen, halo, alkyl, alkenyl, cycloalkyl, cycloalkenyl, aryl, heterocyclyl, heteroaryl, —OR₁₃, —SR₁₃, —(CH₂)ₚCOOR₁₃, —OC(O)R₁₃, —N(R₁₃)₂, —CON(R₁₃)₂, —NO₂, —CN —OC(O)N(R₁₃)₂, and —X,
provided that the radiometal complex comprises at least one X, and when R₁₈ is X, then L₁ is a linker or at least one of R₁₂ and R₁₄-R₁₇ is not hydrogen.
54. The immunoconjugate according to embodiment 8, wherein the radiometal complex is a radiometal complex of Formula (IV-m),
   wherein the radiometal complex of Formula (IV-m) has the following structure:

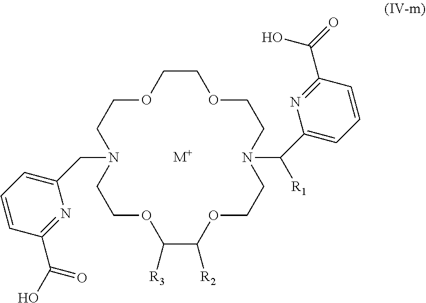

or a pharmaceutically acceptable salt thereof, wherein:
M⁺ is the radiometal, preferably selected from the group consisting of actinium-225(²²⁵Ac), radium-223 (²³³Ra), bismuth-213 (²¹³Bi), lead-212 (²¹²Pb(II) and/or ²¹²Pb(IV)), terbium-149 (¹⁴⁹Tb), terbium-152 (¹⁵²Tb), terbium-155 (¹⁵⁵Tb), fermium-255 (²⁵⁵Fm), thorium-227 (²²⁷Th), thorium-226 (²²⁶Th⁴⁺), astatine-211 (²¹¹At), cerium-134 (¹³⁴Ce), neodymium-144 (¹⁴⁴Nd), lanthanum-132 (¹³²La), lanthanum-135 (¹³⁵La) and uranium-230 (²³⁰U);
R₁ is hydrogen and R₂ is -L₁-R₄;
alternatively, R₁ is -L₁-R₄ and R₂ is hydrogen;
R₃ is hydrogen;
alternatively, R₂ and R₃ are taken together with the carbon atoms to which they are attached to form a 5- or 6-membered cycloalkyl, wherein the 5- or 6-membered cycloalkyl is optionally substituted with -L₁-R₄;
L₁ is absent or a linker;
R₄ comprises the antigen binding domain with binding specificity for kallikrein related peptidase 2 (hK2).
55. The immunoconjugate according to embodiment 8, wherein the radiometal complex is a radiometal complex of Formula (V-m),
   wherein the radiometal complex of Formula (V-m) has the following structure:

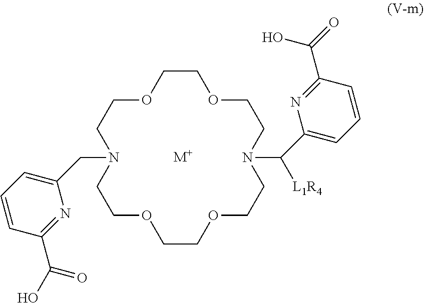

or a pharmaceutically acceptable salt thereof, wherein:
M⁺ is the radiometal, preferably selected from the group consisting of actinium-225(²²⁵Ac), radium-223 (²³³Ra), bismuth-213 (²¹³Bi), lead-212 (²¹²Pb(II) and/or ²¹²Pb(IV)), terbium-149 (¹⁴⁹Tb), terbium-152 (¹⁵²Tb), terbium-155 (¹⁵⁵Tb),fermium-255 (²⁵⁵Fm), thorium-227 (²²⁷Th), thorium-226 (²²⁶Th⁴⁺), astatine-211 (²¹¹At), cerium-134 (¹³⁴Ce), neodymium-144 (¹⁴⁴Nd), lanthanum-132 (¹³²La), lanthanum-135 (¹³⁵La) and uranium-230 (²³⁰U);
L₁ is absent or a linker;
R₄ comprises the antigen binding domain with binding specificity for kallikrein related peptidase 2 (hK2).
56. The immunoconjugate according to embodiment 8, wherein the radiometal complex is a radiometal complex of Formula (VI-m),
   wherein the radiometal complex of Formula (VI-m) has the following structure:

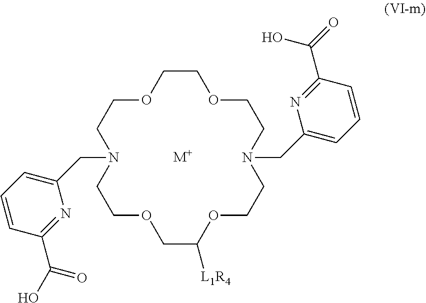

or a pharmaceutically acceptable salt thereof, wherein:
M⁺ is the radiometal, preferably selected from the group consisting of actinium-225(²²⁵Ac), radium-223 (²³³Ra), bismuth-213 (²¹³Bi), lead-212 (²¹²Pb(II) and/or ²¹²Pb(IV)), terbium-149 (¹⁴⁹Tb), terbium-152 (¹⁵²Tb), terbium-155 (¹⁵⁵Tb),fermium-255 (²⁵⁵Fm), thorium-227 (²²⁷Th), thorium-226 (²²⁶Th⁴⁺), astatine-211 (²¹¹At), cerium-134 (¹³⁴Ce), neodymium-144 (¹⁴⁴Nd), lanthanum-132 (¹³²La), lanthanum-135 (¹³⁵La) and uranium-230 (²³⁰U);
L₁ is absent or a linker; and
R₄ comprises the antigen binding domain with binding specificity for kallikrein related peptidase 2 (hK2).
57. An immunoconjugate comprising: a radiometal complex conjugated to an antigen binding domain with binding specificity for kallikrein related peptidase 2 (hK2), wherein the radiometal complex comprises ⁶⁴Cu bound to a chelator, and wherein the chelator is conjugated to the antigen binding domain with binding specificity for kallikrein related peptidase 2 (hK2).
58. The immunoconjugate of embodiment 57, wherein the chelator is 1,4,7,10-tetraazacyclododecane-1,4,7,10,tetraacetic acid (DOTA), S-2-(4-isothiocyanatobenzyl)-1,4,7-triazacyclononane-1,4,7-triacetic acid (NOTA), 1,4,8,11-tetraazacyclodocedan-1,4,8,11-tetraacetic acid (TETA), 3,6,9,15-tetraazabicyclo[9.3.1]-pentadeca-1(15),11,13-triene-4-(S)-(4-isothiocyanatobenzyl)-3,6,9-triacetic acid (PCTA), 5-S-(4-aminobenzyl)-1-oxa-4,7,10-triazacyclododecane-4,7,10-tris(acetic acid) (DO3A), or a derivative thereof.
59. The immunoconjugate of embodiment 57, wherein the chelator is NOTA or a derivative thereof, such as NODA-GA, NODA-GA(tButyl)₃, Di-t-butyl-NOTA, NOTA-thiosemicarbazide, NODA-MPAA, or NODA-MPAEM, preferably NODA-GA.
60. The immunoconjugate according to any of embodiments 57-59, wherein the antigen binding domain with binding specificity for hK2 is a Fab.
61. The immunoconjugate according to any of embodiments 57-60, wherein the antigen binding domain comprises the HCDR1, the HCDR2, the HCDR3, the LCDR1, the LCDR2 and the LCDR3 of SEQ ID NO: 170 (SYYWS), SEQ ID NO: 171 (YIYYSGSTNYNPSLKS), SEQ ID NO: 172 (TTIFGVVTPNFYYGMDV), SEQ ID NO: 173 (RASQGISSYLA), SEQ ID NO: 174 (AASTLQS) and SEQ ID NO: 175 (QQLNSYPLT), respectively.
62. The immunoconjugate according to any of embodiments 57-61, wherein the antigen binding domain that binds hK2 comprises a VH which is at least 80% (e.g. at least 85%, at least 90%, at least 95%, at least 99% or 100%) identical to the VH of SEQ ID NO: 162 (QVQLQESGPGLVKPSETLSLTCTVSGGSISSYYWSWIRQPPGKGLEWIGYIYYSGSTNY NPSLKSRVTISVDTSKNQFSLKLSSVTAADTAVYYCAGTTIFGVVTPNFYYGMDVWGQ GTTVTVSS), and a VL which is at least 80% (e.g. at least 85%, at least 90%, at least 95%, at least 99% or 100%) identical to the VL of SEQ ID NO: 163 (DIQMTQSPSFLSASVGDRVTITCRASQGISSYLAWYQQKPGKAPKFLIYAASTLQSGVPS RFSGSGSGTEFTLTISSLQPEDFATYYCQQLNSYPLTFGGGTKVEIK).
63. The immunoconjugate according to any of embodiments 57-62, wherein the antigen binding domain that binds hK2 comprises the VH of SEQ ID NO: 162 and the VL of SEQ ID NO: 163.
64. The immunoconjugate according to any of embodiments 57-60, wherein the antigen binding domain is a Fab that comprises:
a. a HCDR1, a HCDR2, a HCDR3, a LCDR1, a LCDR2 and a LCDR3 of SEQ ID NOs: 170, 171, 172, 173, 174 and 175, respectively; and/or
b. a VH of SEQ ID NO: 162 and a VL of SEQ ID NO: 163.
65. The immunoconjugate according to any of embodiments 57-64, wherein the immunoconjugate is a short half-life immunoconjugate.
66. An immunoconjugate comprising: a radiometal complex conjugated to an antigen binding domain with binding specificity for kallikrein related peptidase 2 (hK2), wherein:
   the radiometal complex comprises ⁶⁴Cu bound to a chelator that is NOTA or a derivative thereof, such as NODA-GA, NODA-GA(tButyl)₃, Di-t-butyl-NOTA, NOTA-thiosemicarbazide, NODA-MPAA, or NODA-MPAEM, preferably NODA-GA,
   wherein the chelator is conjugated to the antigen binding domain with binding specificity for kallikrein related peptidase 2 (hK2), and
   wherein the antigen binding domain comprises:
   (a) the HCDR1, the HCDR2, the HCDR3, the LCDR1, the LCDR2 and the LCDR3 of SEQ ID NO: 170 (SYYWS), SEQ ID NO: 171 (YIYYSGSTNYNPSLKS), SEQ ID NO: 172 (TTIFGVVTPNFYYGMDV), SEQ ID NO: 173 (RASQGISSYLA), SEQ ID NO: 174 (AASTLQS) and SEQ ID NO: 175 (QQLNSYPLT), respectively; and/or
   (b) a VH which is at least 95%, at least 99% or 100% identical to the VH of SEQ ID NO: 162, and a VL which is at least 95%, at least 99% or 100% identical to the VL of SEQ ID NO: 163.
67. A method of detecting the presence of prostate cancer in a subject, comprising administering an immunoconjugate according to any of embodiments 57-66 to a subject suspected to have prostate cancer and visualizing biological structures to which the conjugate is bound (e.g., by computerized tomography or positron emission tomography), thereby detecting the presence of prostate cancer.
68. A method of detecting the progress of cancer treatment in a subject, comprising administering an immunoconjugate according to any of embodiments 57-66 to the subject and visualizing biological structures to which the immunoconjugate is bound (e.g., by computerized tomography or positron emission tomography), thereby detecting the progress of prostate cancer treatment in the subject.
69. The method of embodiment 67 or embodiment 68 comprising using positron emission tomography (PET) imaging to visualize the biological structures to which the conjugate is bound.
70. The method of any of embodiments 67-69 further comprising administering an anti-cancer therapeutic (e.g., an anti-hK2 therapeutic) to the subject if prostate cancer has been detected in the subject.
71. Use of an immunoconjugate according to any of embodiments 57-66 to diagnose a subject with prostate cancer.
72. Use of an immunoconjugate according to any of embodiments 57-66 to visualize prostate cancer in a patient.
73. Use of an immunoconjugate according to any of embodiments 57-66 to determine the stage of prostate cancer in a subject.

### Chelators

According to particular embodiments, the present invention relates to immunoconjugates, such as radioimmunoconjugates, comprising a chelator, preferably a chelator to which radiometals can be chelated via coordinate bonding. According to particular embodiments, chelators of the invention refer to a chelator to which a metal, preferably a radiometal, can be complexed to form a radiometal complex. Preferably, the chelator is a macrocyclic compound. In certain embodiments, a chelator comprises a macrocycle or a macrocyclic ring containing one or more heteroatoms, e.g., oxygen and/or nitrogen as ring atoms.

According to particular embodiments, the chelator comprises a macrocyclic chelating moiety. Examples of macrocyclic chelating moieties include, without limitation, 1,4,7,10-tetraazacyclododecane-1,4,7,10,tetraacetic acid (DOTA), S-2-(4-isothiocyanatobenzyl)-1,4,7-triazacyclononane-1,4,7-triacetic acid (NOTA), 1,4,8,11-tetraazacyclodocedan-1,4,8,11-tetraacetic acid (TETA), 3,6,9,15-tetraazabicyclo[9.3.1]-pentadeca-1(15),11,13-triene-4-(S)-(4-isothiocyanatobenzyl)-3,6,9-triacetic acid (PCTA), 5-S-(4-aminobenzyl)-1-oxa-4,7,10-triazacyclododecane-4,7,10-tris(acetic acid) (DO3A), or a derivative thereof. In some aspects, the chelator is 1,4,7,10-tetraazacyclododecane-1,4,7,10,tetraacetic acid (DOTA). In other aspects, the chelator is S-2-(4-isothiocyanatobenzyl)-1,4,7-triazacyclononane-1,4,7-triacetic acid (NOTA). In further aspects, the chelator is 1,4,8,11-tetraazacyclodocedan-1,4,8,11-tetraacetic acid (TETA). In yet other aspects, the chelator is 3,6,9,15-tetraazabicyclo[9.3.1]-pentadeca-1(15),11,13-triene-4-(S)-(4-isothiocyanatobenzyl)-3,6,9-triacetic acid (PCTA). In still further aspects, the chelator is 5-S-(4-aminobenzyl)-1-oxa-4,7,10-triazacyclododecane-4,7,10-tris(acetic acid) (DO3A). In other aspects, the chelator is DOTA, DFO, DTPA, NOTA, or TETA.

In certain embodiments the chelator comprises NOTA or a derivative thereof. Non-limiting examples of NOTA derivatives include NODA, NODA-GA, NODA-GA(tButyl)₃, Di-t-butyl-NOTA, NOTA-thiosemicarbazide, NODA-MPAA, and NODA-MPAEM. In an embodiment, the conjugatable version of the chelator-linker may be referred to as NODA-GA-NHS, or 2,2′,2″-(1,4,7-triazacyclononane-1,4,7-triyl)diacetic acid-glutamic acid, as illustrated below:

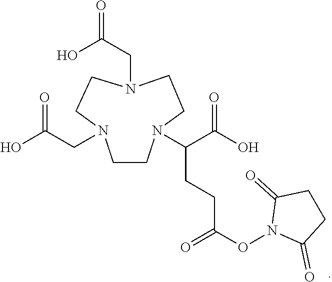 [SMILES: O=C(O)CN1CCN(CC(=O)O)CCN(C(CCC(=O)ON2C(=O)CCC2=O)C(=O)O)CC1]

In certain embodiments, the chelator comprises a macrocycle that is a derivative of 4,13-diaza-18-crown-6. 4,13-Diaza-18-crown-6 may be prepared in a variety of ways (See, e.g., Gatto et al., Org. Synth. 1990, 68, 227; DOI: 10.15227/orgsyn.068.0227). According to further embodiments of the present invention, the chelator is H₂bp18c6 or a H₂bp18c6 derivative, such as those described in WO2020/229974. H₂bp18c6 refers to N,N′-bis[(6-carboxy-2-pyridil)methyl]-4,13-diaza-18-crown-6, as described herein. H₂bp18c6 and H₂bp18c6 derivatives are also described, for example, in Thiele et al. “An Eighteen-Membered Macrocyclic Ligand for Actinium-225 Targeted Alpha Therapy” Angew. Chem. Int. Ed. (2017) 56, 14712-14717, and Roca-Sabio et al. “Macrocyclic Receptor Exhibiting Unprecedented Selectivity for Light Lanthanides” J. Am. Chem. Soc. (2009) 131, 3331-3341, which are incorporated by reference herein. Additional chelators suitable for use in accordance with the present invention are described in WO2018/183906 and WO2020/106886, which are incorporated by reference herein. As used herein, the term “TOPA” refers to the macrocycle known in the art as H₂bp18c6 and may alternatively be referred to as N,N′-bis[(6-carboxy-2-pyridil)methyl]-4,13-diaza-18-crown-6 (see, e.g., Roca-Sabio et al.).

### CHELATORS OF FORMULAE (I), (II) and (III)

Additional chelators suitable for use in accordance with the present invention are described in WO2020/229974, which is incorporated by reference herein. According to particular embodiments, e.g., as described in WO2020/229974, the chelator has the structure of Formula (I):

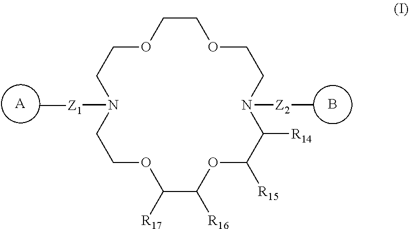

wherein:
   each of ring A and ring B is independently a 6-10 membered aryl or a 5-10 membered heteroaryl, wherein each of ring A and ring B is optionally substituted with one or more substituents independently selected from the group consisting of halo, alkyl, alkenyl, cycloalkyl, cycloalkenyl, aryl, heterocyclyl, heteroaryl, —OR₁₃, —SR₁₃, —(CH₂)ₚCOOR₁₃, —OC(O)R₁₃, —N(R₁₃)₂, —CON(R₁₃)₂, —NO₂, —CN —OC(O)N(R₁₃)₂, and X;
   each of Z₁ and Z₂ is independently —(C(R₁₂)₂)ₘ— or —(CH₂)ₙ—C(R₁₂)(X)—(CH₂)ₙ—;
   each X is independently -L₁-R₁₁;
   each n is independently 0, 1, 2, 3, 4, or 5;
   each m is independently 1, 2, 3, 4, or 5;
   each p is independently 0 or 1;
   L₁ is absent or a linker;
   R₁₁ is a nucleophilic moiety or an electrophilic moiety, or R₁₁ comprises an antigen binding domain;
   each R₁₂ is independently hydrogen, alkyl, cycloalkyl, aryl, heterocyclyl, or heteroaryl;
   each R₁₃ is independently hydrogen or alkyl;
   each of R₁₄, R₁₅, R₁₆, and R₁₇ is independently hydrogen, alkyl, or X,
   or alternatively R₁₄ and R₁₅ and/or R₁₆ and R₁₇ are taken together with the carbon atoms to which they are attached to form a 5- or 6-membered cycloalkyl ring optionally substituted with X;
   provided that the chelator comprises at least one X, and when X is present on ring A or ring B, L₁ is a linker or at least one of R₁₂ and R₁₄-R₁₇ is not hydrogen.

According to embodiments of the invention, a chelator comprises at least one X group, wherein X is -L₁-R₁₁, wherein L₁ is absent or a linker, and R₁₁ is an electrophilic moiety or a nucleophilic moiety, or R₁₁ comprises an antigen binding domain. When R₁₁ is a nucleophilic or electrophilic moiety, such moiety can be used for attachment of the chelator to an antigen binding domain, directly or indirectly via a linker. According to preferred embodiments, R₁₁ comprises an antibody or antigen binding domain with binding specificity for hK2, such as the Fab of KL2B30.

In certain embodiments, a chelator comprises a single X group, and preferably L₁ of the X group is a linker.

A chelator of the invention can be substituted with X at any one of the carbon atoms of the macrocyclic ring, the Z₁ or Z₂ position, or on ring A or ring B, provided that when ring or ring B comprises an X group, L₁ is a linker or at least one of R₁₂ and R₁₄-R₁₇ is not hydrogen (i.e., at least one of the carbon atoms of Z₁, Z₂, and/or the carbons of the macrocyclic ring is substituted for instance with an alkyl group, such as methyl or ethyl). Preferably, substitution at such positions does not affect the chelation efficiency of the chelator for radiometal ions, particularly ²²⁵Ac, and in some embodiments, the substitutions can enhance chelation efficiency.

In some embodiments, L₁ is absent. When L₁ is absent, Ru is directly bound (e.g., via covalent linkage) to the chelator.

In some embodiments, L₁ is a linker. As used herein, the term “linker” refers to a chemical moiety that joins a chelator to a nucleophilic moiety, electrophilic moiety, or antigen binding domain. Any suitable linker known to those skilled in the art in view of the present disclosure can be used in the invention. The linkers can contain, for example, a substituted or unsubstituted alkyl, a substituted or unsubstituted heteroalkyl moiety, a substituted or unsubstituted aryl or heteroaryl, a polyethylene glycol (PEG) linker, a peptide linker, a sugar-based linker, or a cleavable linker, such as a disulfide linkage or a protease cleavage site such as valine-citrulline-p-aminobenzyl (PAB). Exemplary linker structures suitable for use in the invention include, but are not limited to:

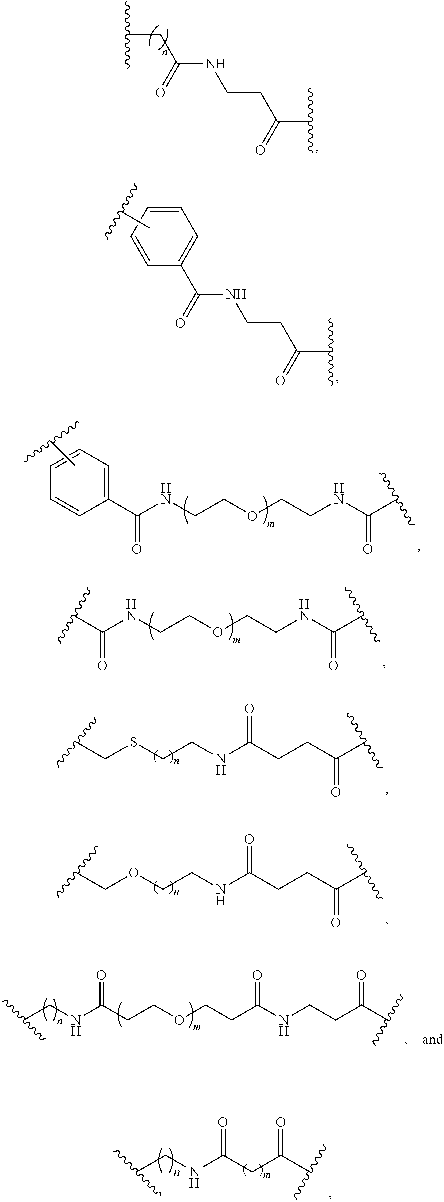

wherein n is an integer of 0 to 10, preferably an integer of 1 to 4; and m is an integer of 0 to 12, preferably an integer of 0 to 6.

In some embodiments, R₁₁ is a nucleophilic moiety or an electrophilic moiety. A “nucleophilic moiety” or “nucleophilic group” refers to a functional group that donates an electron pair to form a covalent bond in a chemical reaction. An “electrophilic moiety” or “electrophilic group” refers to a functional group that accepts an electron pair to form a covalent bond in a chemical reaction. Nucleophilic groups react with electrophilic groups, and vice versa, in chemical reactions to form new covalent bonds. Reaction of the nucleophilic group or electrophilic group of a chelator of the invention with an antigen binding domain or other chemical moiety (e.g., linker) comprising the corresponding reaction partner allows for covalent linkage of the antigen binding domain or chemical moiety to the chelator of the invention.

Exemplary examples of nucleophilic groups include, but are not limited to, azides, amines, and thiols. Exemplary examples of electrophilic groups include, but are not limited to amine-reactive groups, thiol-reactive groups, alkynyls and cycloalkynyls. An amine-reactive group preferably reacts with primary amines, including primary amines that exist at the N-terminus of each polypeptide chain and in the side-chain of lysine residues. Examples of amine-reactive groups suitable for use in the invention include, but are not limited to, N-hydroxy succinimide (NHS), substituted NHS (such as sulfo-NHS), isothiocyanate (—NCS), isocyanate (—NCO), esters, carboxylic acid, acyl halides, amides, alkylamides, and tetra- and per-fluoro phenyl ester. A thiol-reactive group reacts with thiols, or sulfhydryls, preferably thiols present in the side-chain of cysteine residues of polypeptides. Examples of thiol-reactive groups suitable for use in the invention include, but are not limited to, Michael acceptors (e.g., maleimide), haloacetyl, acyl halides, activated disulfides, and phenyloxadiazole sulfone.

In particular embodiments, R₁₁ is —NH₂, —NCS (isothiocyanate), —NCO (isocyanate), —N₃ (azido), alkynyl, cycloalkynyl, carboxylic acid, ester, amido, alkylamide, maleimido, acyl halide, tetrazine, or trans-cyclooctene, more particularly —NCS, —NCO, —N₃, alkynyl, cycloalkynyl, —C(O)R₁₃, —COOR₁₃, —CON(R₁₃)₂, maleimido, acyl halide (e.g., —C(O)Cl, —C(O)Br), tetrazine, or trans-cyclooctene wherein each R₁₃ is independently hydrogen or alkyl.

In some embodiments, R₁₁ is an alkynyl, cycloalkynyl, or azido group thus allowing for attachment of the chelator to an antigen binding domain or other chemical moiety (e.g., linker) using a click chemistry reaction. In such embodiments, the click chemistry reaction that can be performed is a Huisgen cycloaddition or 1,3-dipolar cycloaddition between an azido (—N₃) and an alkynyl or cycloalkynyl group to form a 1,2,4-triazole linker or moiety. In one embodiment, the chelator comprises an alkynyl or cycloalkynyl group and the antigen binding domain or other chemical moiety comprises an azido group. In another embodiment, the chelator comprises an azido group and the antigen binding domain or other chemical moiety comprises an alkynyl or cycloalkynyl group.

In certain embodiments, R₁₁ is an alkynyl group, more preferably a terminal alkynyl group or cycloalkynyl group that is reactive with an azide group, particularly via strain-promoted azide-alkyne cycloaddition (SPAAC). Examples of cycloalkynyl groups that can react with azide groups via SPAAC include, but are not limited to cyclooctynyl or a bicyclononynyl (BCN), difluorinated cyclooctynyl (DIFO), dibenzocyclooctynyl (DIBO), keto-DIBO, biarylazacyclooctynonyl (BARAC), dibenzoazacyclooctynyl (DIBAC, DBCO, ADIBO), dimethoxyazacyclooctynyl (DIMAC), difluorobenzocyclooctynyl (DIFBO), monobenzocyclooctynyl (MOBO), and tetramethoxy dibenzocyclooctynyl (TMDIBO).

In a particular embodiment, R₁₁ is dibenzoazacyclooctynyl (DIBAC, DBCO, ADIBO), which has the following structure:

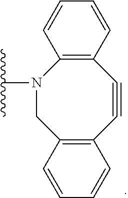 [SMILES: *N1Cc2ccccc2C#Cc2ccccc21]

In such embodiments in which R₁₁ is DBCO, the DBCO can be covalently linked to a chelator directly or indirectly via a linker, and is preferably attached to the chelator indirectly via a linker.

In some embodiments, R₁₁ comprises an antigen binding domain. The antigen binding domain can be linked to the chelator directly via a covalent linkage, or indirectly via a linker. In preferred embodiments, the antigen binding domain is an antibody or antigen binding fragment thereof. According to preferred embodiments, R₁₁ comprises an antigen binding domain with binding specificity for hK2, such as the Fab of KL2B30.

According to embodiments of the invention, each of ring A and ring B is independently a 6-10 membered aryl or a 5-10 membered heteroaryl. In alternative embodiments, it is contemplated that each of ring A and ring B is an optionally substituted heterocyclyl ring, such as oxazoline. Each of ring A and ring B can be optionally and independently substituted with one or more substituent groups independently selected from the group consisting of halo, alkyl, alkenyl, cycloalkyl, cycloalkenyl, aryl, heterocyclyl, heteroaryl, —OR₁₃, —SR₁₃, —(CH₂)ₚCOOR₁₃, —OC(O)R₁₃, —N(R₁₃)₂, —CON(R₁₃)₂, —NO₂, —CN —OC(O)N(R₁₃)₂, and X. Examples of 6-10 membered aryl groups suitable for this purpose include, but are not limited to, phenyl and naphthyl. Examples of 5 to 10 membered heteroaryl groups suitable for this purpose include, but are not limited to pyridinyl, thiazolyl, isothiazolyl, oxazolyl, isoxazolyl, and imidazolyl. Examples of suitable substituents of the 5 to 10 membered heteroaryl and 6 to 10 membered aryl groups include, but are not limited to —COOH, tetrazolyl, and —CH₂COOH. In preferred embodiments, a substituent group is —COOH or tetrazolyl, which is an isostere of —COOH.

In certain embodiments, each of ring A and ring B are independently and optionally substituted with one or more carboxyl groups, including but not limited to, —COOH and —CH₂COOH.

In certain embodiments, each of ring A and ring B are independently and optionally substituted with tetrazolyl.

In one embodiment, ring A and ring B are the same, e.g., both ring A and ring B are pyridinyl. In another embodiment, ring A and ring B are different, e.g., one of ring A and ring is pyridinyl and the other is phenyl.

In a particular embodiment, both ring A and ring B are pyridinyl substituted with —COOH.

In a particular embodiment, both ring A and ring B are pyridinyl substituted with tetrazolyl.

In another particular embodiment, both ring A and ring B are picolinic acid groups having the following structure:

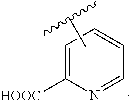

According to embodiments of the invention, each of Z₁ and Z₂ is independently —(C(R₁₂)₂)ₘ— or —(CH₂)ₙ—C(R₁₂)(X)—(CH₂)ₙ—; each X is independently -L₁-R₁₁; each R₁₂ is independently hydrogen, alkyl, cycloalkyl, aryl, heterocyclyl, or heteroaryl; each n is independently 0, 1, 2, 3, 4, or 5; and each m is independently 1, 2, 3, 4, or 5.

In some embodiments, each R₁₂ is independently hydrogen or alkyl, more preferably hydrogen, —CH₃, or —CH₂CH₃.

In some embodiments, each R₁₂ is hydrogen.

In some embodiments, both Z₁ and Z₂ are —(CH₂)ₘ—, wherein each m is preferably 1. In such embodiments, a carbon atom of the macrocyclic ring, ring A, or ring B is substituted with an X group.

In some embodiments, one of Z₁ and Z₂ is —(CH₂)ₙ—C(R₁₂)(X)—(CH₂)ₙ— and the other is —(CH₂)ₘ—.

In some embodiments, one of Z₁ and Z₂ —(CH₂)ₙ—C(R₁₂)(X)—(CH₂)ₙ— and the other is —(CH₂)ₘ—; each n is 0; m is 1; X is -L₁-R₁₁; and L₁ is a linker.

In some embodiments, both Z₁ and Z₂ are —(CH₂)ₘ—; each m is independently 0, 1, 2, 3, 4, or 5, preferably each m is 1; and one of R₁₄, R₁₅, R₁₆, and R₁₇ is X, and the rest of R₁₄, R₁₅, R₁₆, and R₁₇ are each hydrogen.

In some embodiments, R₁₄ and R₁₅ are taken together with the carbon atoms to which they are attached to form a 5- or 6-membered cycloalkyl ring (i.e., cyclopentyl or cyclohexyl). Such 5- or 6-membered cycloalkyl ring can be substituted with an X group.

In some embodiments R₁₆ and R₁₇ are taken together with the carbon atoms to which they are attached to form a 5- or 6-membered cycloalkyl ring (i.e., cyclopentyl or cyclohexyl). Such 5- or 6-membered cycloalkyl ring can be substituted with an X group.

In certain embodiments, a chelator has the structure of Formula (II):

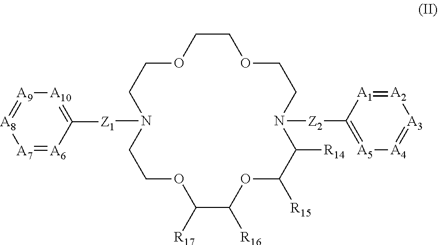

wherein:
   A₁ is N or CR₁ or is absent;
   A₂ is N or CR₂;
   A₃ is N or CR₃;
   A₄ is N or CR₄;
   A₅ is N or CR₅;
   A₆ is N or CR₆ or is absent;
   A₇ is N or CR₇;
   A₈ is N or CR₈;
   A₉ is N or CR₉;
   A₁₀ is N or CR₁₀;
provided that no more than three of A₁, A₂, A₃, A₄, and A₅ are N, and no more than three of A₆, A₇, A₈, A₉, and A₁₀ are N;
   each of R₁, R₂, R₃, R₄, R₅, R₆, R₇, R₈, R₉, and R₁₀ is independently selected from the group consisting of hydrogen, halo, alkyl, alkenyl, cycloalkyl, cycloalkenyl, aryl, heterocyclyl, heteroaryl, —OR₁₃, —SR₁₃, —(CH₂)ₚCOOR₁₃, —OC(O)R₁₃, —N(R₁₃)₂, —CON(R₁₃)₂, —NO₂, —CN, —OC(O)N(R₁₃)₂, and —X,
   or, alternatively, any two directly adjacent R₁, R₂, R₃, R₄, R₅, R₆, R₇, R₈, R₉, and R₁₀ are taken together with the atoms to which they are attached to form a five or six-membered substituted or unsubstituted carbocyclic or nitrogen-containing ring;
   and Z₁, Z₂, X, n, m, p, L₁, and R₁₁—R₁₇ are as described above for formula (I),
   provided that the chelator comprises at least one X, and when any one of R₁, R₂, R₃, R₄, R₅, R₆, R₇, R₈, R₉, and R₁₀ is X, then L₁ is a linker or at least one of R₁₂ and R₁₄-R₁₇ is not hydrogen.

In some embodiments, any two directly adjacent R₁, R₂, R₃, R₄, R₅, R₆, R₇, R₈, R₉, and R₁₀ are taken together with the atoms to which they are attached to form a five or six-membered substituted or unsubstituted carbocyclic or nitrogen-containing ring. Examples of such carbocyclic rings that can be formed include, but are not limited to, naphthyl. Examples of such nitrogen-containing rings that can be formed include, but are not limited to, quinolinyl. The carbocyclic or nitrogen-containing rings can be unsubstituted or substituted with one or more suitable substituents, e.g., —COOH, —CH₂COOH, tetrazolyl etc.

In some embodiments, L₁ is absent. When L₁ is absent, Ru is directly bound (e.g., via covalent linkage) to the chelator.

In some embodiments, L₁ is a linker. Any suitable linker known to those skilled in the art in view of the present disclosure can be used in the invention, such as those described above.

In some embodiments, one of A₁, A₂, A₃, A₄, and A₅ is nitrogen, one of A₁, A₂, A₃, A₄, and A₅ is carbon substituted with —COOH and the rest are CH, i.e., forming a pyridinyl ring substituted with carboxylic acid.

In some embodiments, one of A₆, A₇, A₈, A₉, and A₁₀ is nitrogen, one of A₆, A₇, A₈, A₉, and A₁₀ is carbon substituted with —COOH, and the rest are CH, i.e., forming a pyridinyl ring substituted with carboxylic acid.

In one embodiment, at least one of R₁, R₂, R₃, R₄ and R₅ is —COOH. In one embodiment, at least one of R₆, R₇, R₈, R₉, and R₁₀ is —COOH. In another embodiment, at least one of R₁, R₂, R₃, R₄ and R₅ is —COOH; and at least one of R₆, R₇, R₈, R₉, and R₁₀ is —COOH.

In some embodiments, each of A₁ and A₁₀ is nitrogen; A₂ is CR₂ and R₂ is —COOH; A₉ is CR₉ and R₉ is —COOH; each of A₃-A₈ is CR₂, CR₃, CR₄, CR₅, CR₆, CR₇, and CR₈, respectively; and each of R₃ to R₈ is hydrogen.

In some embodiments, one of A₁, A₂, A₃, A₄, and A₅ is nitrogen, one of A₁, A₂, A₃, A₄, and A₅ is carbon substituted with tetrazolyl and the rest are CH.

In some embodiments, one of A₆, A₇, A₈, A₉, and A₁₀ is nitrogen, one of A₆, A₇, A₈, A₉, and A₁₀ is carbon substituted with tetrazolyl, and the rest are CH.

In one embodiment, at least one of R₁, R₂, R₃, R₄ and R₅ is tetrazolyl. In one embodiment, at least one of R₆, R₇, R₈, R₉, and R₁₀ is tetrazolyl. In another embodiment, at least one of R₁, R₂, R₃, R₄ and R₅ is tetrazolyl; and at least one of R₆, R₇, R₈, R₉, and R₁₀ is tetrazolyl.

In some embodiments, each R₁₂ is hydrogen.

In some embodiments, R₁₁ is an alkynyl group or cycloalkynyl group, preferably cyclooctynyl or a cyclooctynyl derivative, e.g., DBCO.

In particular embodiments of a chelator of formula (II):

each of A₁ and A₁₀ is nitrogen;

A₂ is CR₂ and R₂ is —COOH;

A₉ is CR₉ and R₉ is —COOH;

each of A₃-A₈ is CR₂, CR₃, CR₄, CR₅, CR₆, CR₇, and CR₈, respectively;

each of R₃ to R₈ is hydrogen;

one of Z₁ and Z₂ is —(CH₂)ₘ— and the other of Z₁ and Z₂ is —(CH₂)ₙ—C(R₁₂)(X)—(CH₂)ₙ—;

R₁₂ is hydrogen;

m is 1;

each n is 0;

X is -L₁-R₁₁, wherein L₁ is a linker and —R₁₁ is an electrophilic group, e.g., cyclooctynyl or cyclooctynyl derivative such as DBCO; and

each of R₁₄-R₁₇ is hydrogen, or alternatively R₁₆ and R₁₇ are taken together with the carbon atoms to which they are attached to form a 5- or 6-membered cycloalkyl ring.

In certain embodiments, a chelator has the structure of formula (III):

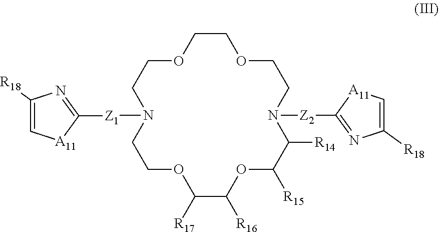

wherein:
   each A₁₁ is independently 0, S, NMe, or NH;
   each R₁₈ is independently selected from the group consisting of hydrogen, halo, alkyl, alkenyl, cycloalkyl, cycloalkenyl, aryl, heterocyclyl, heteroaryl, —OR₁₃, —SR₁₃, —COOR₁₃, —OC(O)R₁₃, —N(R₁₃)₂, —CON(R₁₃)₂, —NO₂, —CN —OC(O)N(R₁₃)₂, and —X,
   and Z₁, Z₂, X, n, m, L₁, R₁₁—R₁₇ are as described above for formula (I),
   provided that the chelator comprises at least one X, and when R₁₈ is X, then L₁ is a linker or at least one of R₁₂ and R₁₄-R₁₇ is not hydrogen.

In some embodiments, each A₁₁ is the same, and each A₁₁ is 0, S, NMe, or NH. For example, each A₁₁ can be S. In other embodiments, each A₁₁ is different and each is independently selected from O, S, NMe, and NH.

In some embodiments, each R₁₈ is independently —(CH₂)ₚ—COOR₁₃ or tetrazolyl, wherein R₁₃ is hydrogen and each p is independently 0 or 1.

In some embodiments, each R₁₈ is —COOH.

In some embodiments, each R₁₈ is —CH₂COOH.

In some embodiments, each R₁₈ is tetrazolyl.

In particular embodiments of a chelator of formula (III):
each R₁₈ is COOH;
one of Z₁ and Z₂ is —(CH₂)ₘ— and the other of Z₁ and Z₂ is —(CH₂)ₙ—C(R₁₂)(X)—(CH₂)ₙ—;
R₁₂ is hydrogen;
m is 1; each n is 0;
X is -L₁-R₁₁, wherein L₁ is a linker and —R₁₁ is an electrophilic group, e.g., cyclooctynyl or cyclooctynyl derivative such as DBCO, or BCN; and
each of R₁₄-R₁₇ is hydrogen, or alternatively R₁₆ and R₁₇ are taken together with the carbon atoms to which they are attached to form a 5- or 6-membered cycloalkyl ring.

In particular embodiments of the invention, a chelator is selected from the group consisting of:

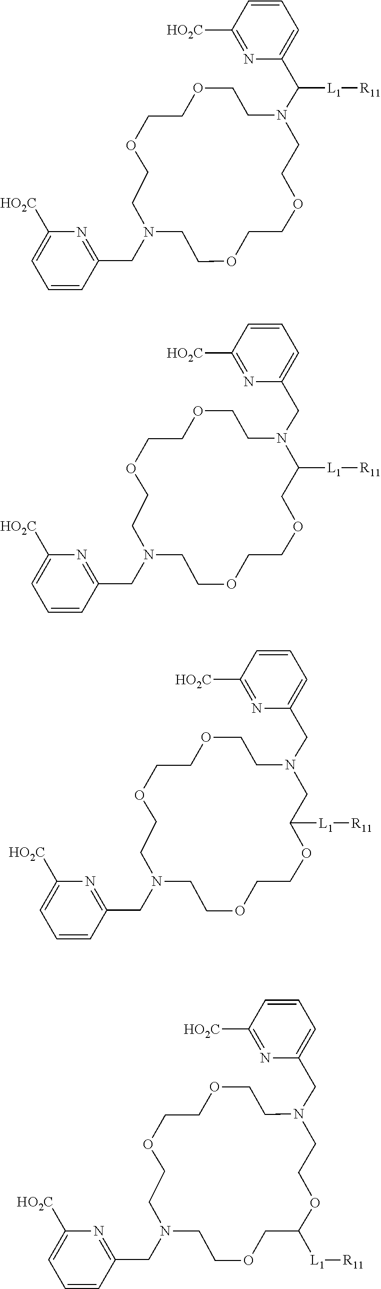

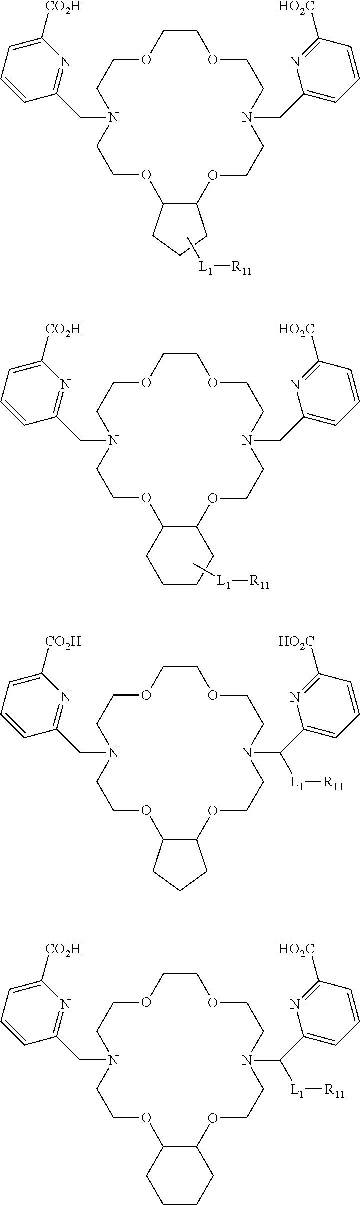

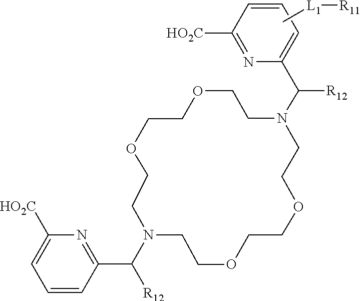

wherein:
   L₁ is absent or a linker;
   R₁ is a nucleophilic moiety or an electrophilic moiety, or R₁ comprises an antigen binding domain (e.g., the Fab of KL2B30); and
   each R₁₂ is independently hydrogen, —CH₃, or —CH₂CH₃, provided at least one R₁₂ is —CH₃ or —CH₂CH₃.

In some embodiments, R₁ is —NH₂, —NCS, —NCO, —N₃, alkynyl, cycloalkynyl, —C(O)R₁₃, —COOR₁₃, —CON(R₁₃)₂, maleimido, acyl halide, tetrazine, or trans-cyclooctene.

In certain embodiments, R₁₁ is cyclooctynyl or a cyclooctynyl derivative selected from the group consisting of bicyclononynyl (BCN), difluorinated cyclooctynyl (DIFO), dibenzocyclooctynyl (DIBO), keto-DIBO, biarylazacyclooctynonyl (BARAC), dibenzoazacyclooctynyl (DIBAC, DBCO, ADIBO), dimethoxyazacyclooctynyl (DIMAC), difluorobenzocyclooctynyl (DIFBO), monobenzocyclooctynyl (MOBO), and tetramethoxy dibenzocyclooctynyl (TMDIBO).

Preferably, R₁₁ is an alkynyl group or cycloalkynyl group, more preferably a cycloalkynyl group, e.g., DBCO or BCN.

Exemplary chelators of the invention include, but are not limited to:

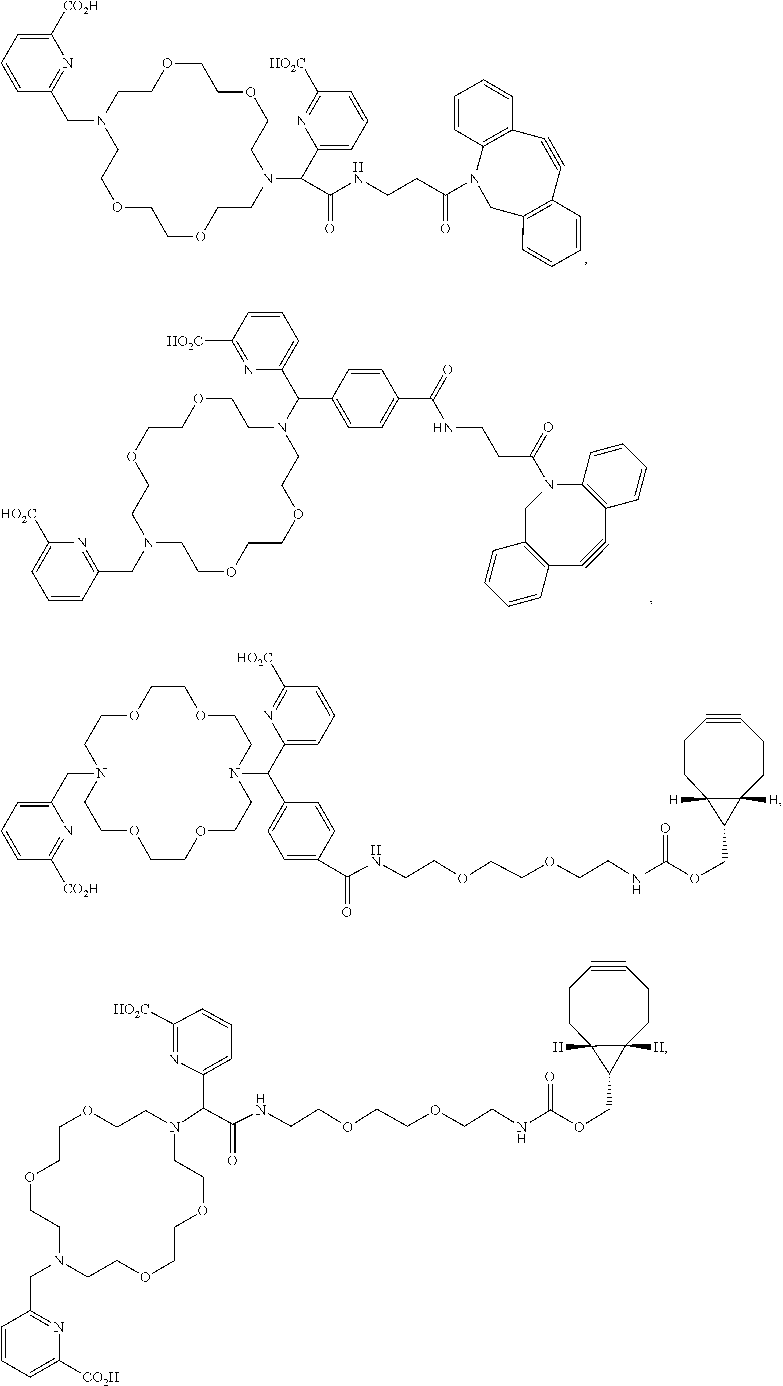

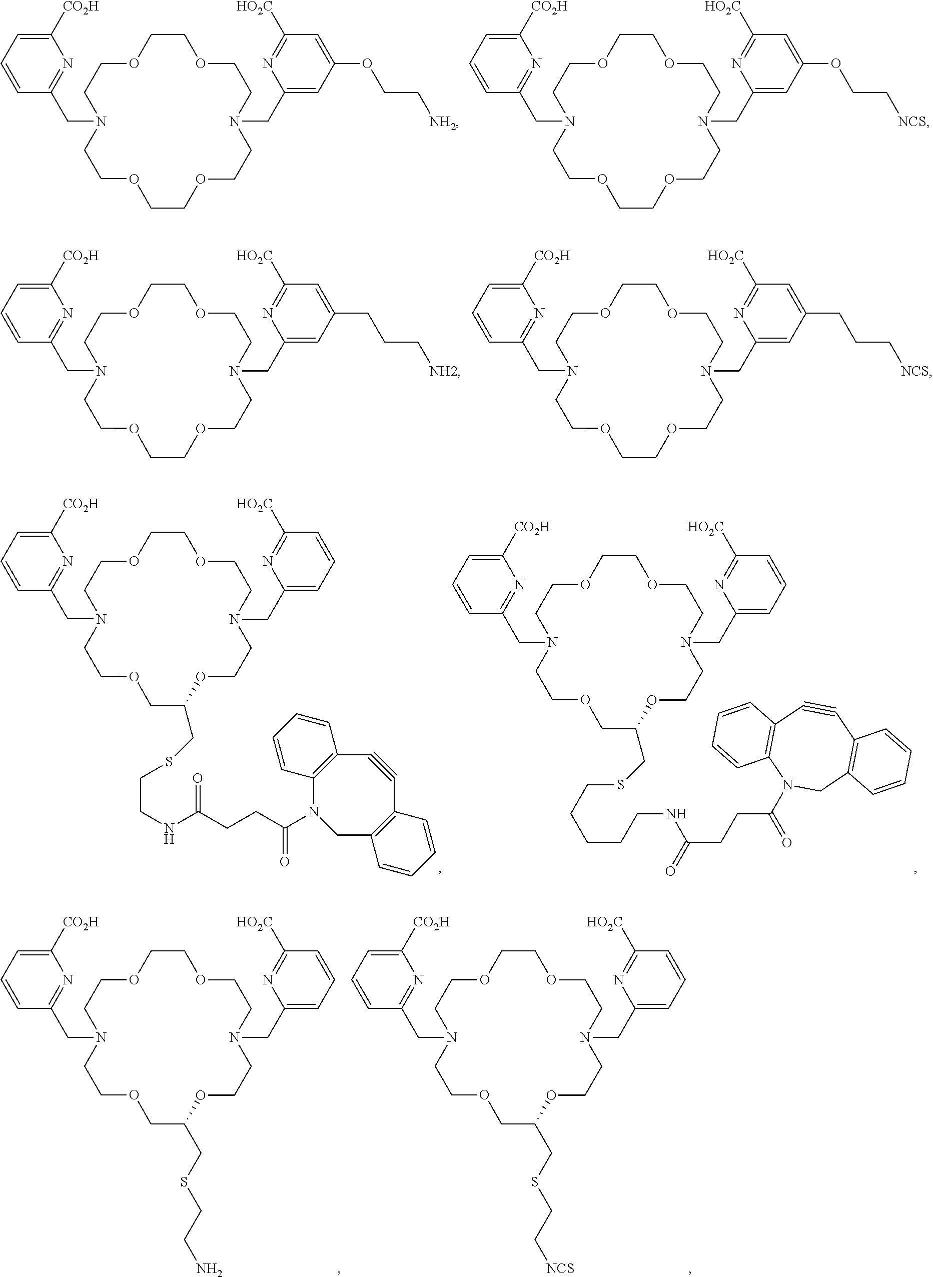

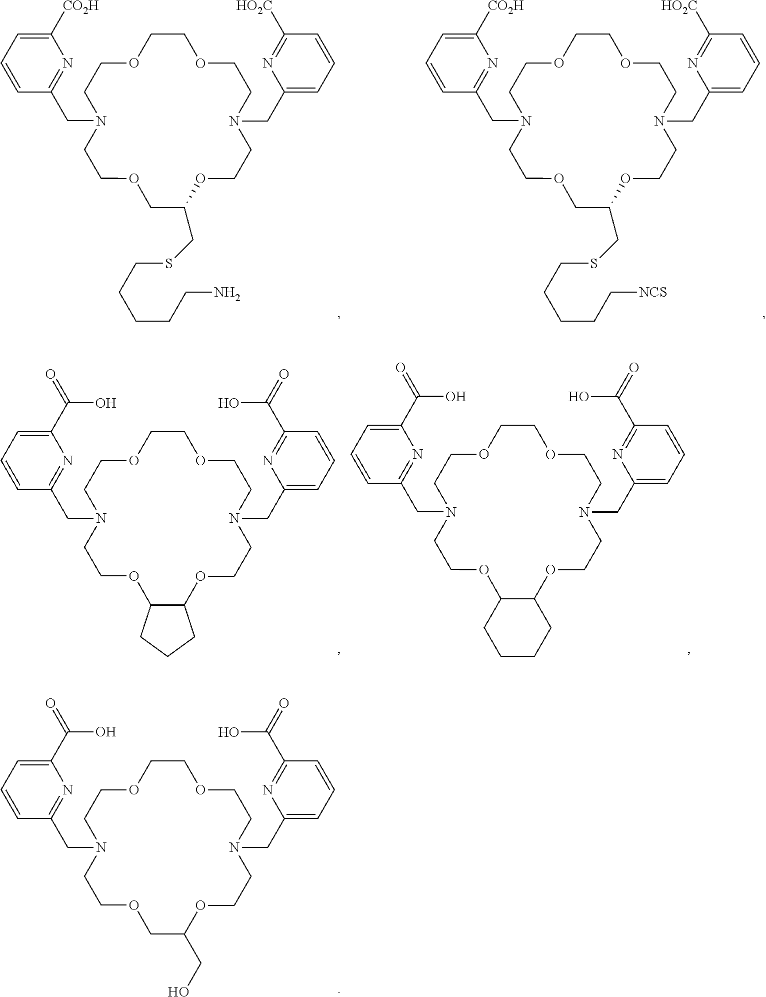

Such chelators can be covalently attached to an antigen binding domain to form immunoconjugates or radioimmunoconjugates by reacting the chelator with an azide-labeled antigen binding domain to form a 1,2,3-triazole linker via a click chemistry reaction as described in WO2020/229974.

Chelators of the invention can be produced by any method known in the art in view of the present disclosure. For example, the pendant aromatic/heteroaromatic groups can be attached to the macrocyclic ring portion by methods known in the art, such as those exemplified and described in WO2020/229974.

### CHELATORS OF FORMULAE (IV), (V) and (VI)

In an embodiment, the chelator is directed to a compound of formula (IV)

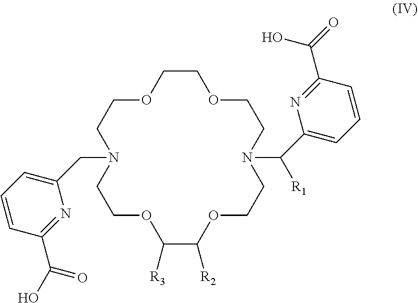

or a pharmaceutically acceptable salt thereof, wherein:
   R₁ is hydrogen and R₂ is -L₁-R₄;
   alternatively, R₁ is -L₁-R₄ and R₂ is hydrogen;
   R₃ is hydrogen;
   alternatively, R₂ and R₃ are taken together with the carbon atoms to which they are attached to form a 5- or 6-membered cycloalkyl, wherein the 5- or 6-membered cycloalkyl is optionally substituted with -L₁-R₄;
   L₁ is absent or a linker; and
   R₄ is a nucleophilic moiety, an electrophilic moiety, or an antigen binding domain (e.g., the Fab of KL2B30).

In some embodiments, L₁ is absent. When L₁ is absent, R₄ is directly bound (e.g., via covalent linkage) to the compound.

In some embodiments, L₁ is a linker. As used herein, the term “linker” refers to a chemical moiety that joins a compound of the invention to a nucleophilic moiety, electrophilic moiety, or antigen binding domain. Any suitable linker known to those skilled in the art in view of the present disclosure can be used in the invention. The linkers can have, for example, a substituted or unsubstituted alkyl, a substituted or unsubstituted heteroalkyl moiety, a substituted or unsubstituted aryl or heteroaryl, a polyethylene glycol (PEG) linker, a peptide linker, a sugar-based linker, or a cleavable linker, such as a disulfide linkage or a protease cleavage site such as valine-citrulline-p-aminobenzyl (PAB). Exemplary linker structures suitable for use in the invention include, but are not limited to:

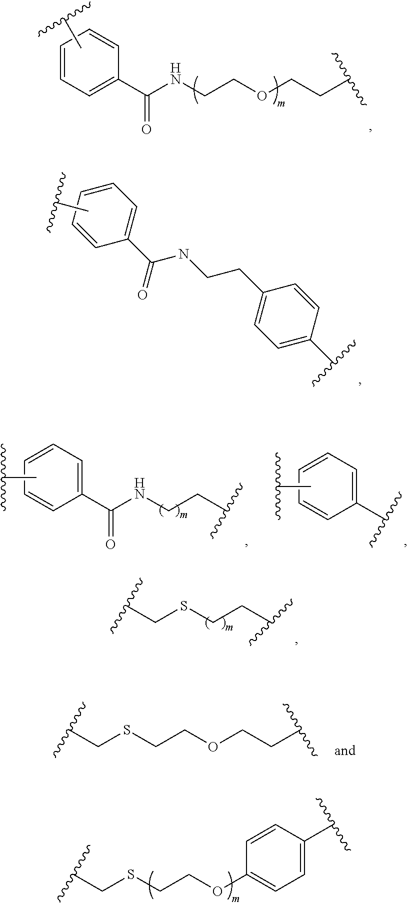

wherein m is an integer of 0 to 12.

In some embodiments, R₄ is a nucleophilic moiety or an electrophilic moiety. A “nucleophilic moiety” or “nucleophilic group” refers to a functional group that donates an electron pair to form a covalent bond in a chemical reaction. An “electrophilic moiety” or “electrophilic group” refers to a functional group that accepts an electron pair to form a covalent bond in a chemical reaction. Nucleophilic groups react with electrophilic groups, and vice versa, in chemical reactions to form new covalent bonds. Reaction of the nucleophilic group or electrophilic group of a compound of the invention with an antigen binding domain or other chemical moiety (e.g., linker) comprising the corresponding reaction partner allows for covalent linkage of the antigen binding domain or chemical moiety to the compound of the invention.

Exemplary examples of nucleophilic groups include, but are not limited to, azides, amines, and thiols. Exemplary examples of electrophilic groups include, but are not limited to amine-reactive groups, thiol-reactive groups, alkynyls and cycloalkynyls. An amine-reactive group preferably reacts with primary amines, including primary amines that exist at the N-terminus of each polypeptide chain and in the side-chain of lysine residues. Examples of amine-reactive groups suitable for use in the invention include, but are not limited to, N-hydroxy succinimide (NHS), substituted NHS (such as sulfo-NHS), isothiocyanate (—NCS), isocyanate (—NCO), esters, carboxylic acid, acyl halides, amides, alkylamides, and tetra- and per-fluoro phenyl ester. A thiol-reactive group reacts with thiols, or sulfhydryls, preferably thiols present in the side-chain of cysteine residues of polypeptides. Examples of thiol-reactive groups suitable for use in the invention include, but are not limited to, Michael acceptors (e.g., maleimide), haloacetyl, acyl halides, activated disulfides, and phenyloxadiazole sulfone.

In certain embodiments, R₄ is —NH₂, —NCS (isothiocyanate), —NCO (isocyanate), —N₃ (azido), alkynyl, cycloalkynyl, carboxylic acid, ester, amido, alkylamide, maleimido, acyl halide, tetrazine, or trans-cyclooctene, more particularly —NCS, —NCO, —N₃, alkynyl, cycloalkynyl, —C(O)R₁₃, —COOR₁₃, —CON(R₁₃)₂, maleimido, acyl halide (e.g., —C(O)Cl, —C(O)Br), tetrazine, or trans-cyclooctene wherein each R₁₃ is independently hydrogen or alkyl.

In some embodiments, R₄ is an alkynyl, cycloalkynyl, or azido group thus allowing for attachment of the compound of the invention to an antigen binding domain or other chemical moiety (e.g., linker) using a click chemistry reaction. In such embodiments, the click chemistry reaction that can be performed is a Huisgen cycloaddition or 1,3-dipolar cycloaddition between an azido (—N₃) and an alkynyl or cycloalkynyl group to form a 1,2,4-triazole linker or moiety. In one embodiment, the compound of the invention comprises an alkynyl or cycloalkynyl group and the antigen binding domain or other chemical moiety comprises an azido group. In another embodiment, the compound of the invention comprises an azido group and the antigen binding domain or other chemical moiety comprises an alkynyl or cycloalkynyl group.

In certain embodiments, R₄ is an alkynyl group, more preferably a terminal alkynyl group or cycloalkynyl group that is reactive with an azide group, particularly via strain-promoted azide-alkyne cycloaddition (SPAAC). Examples of cycloalkynyl groups that can react with azide groups via SPAAC include, but are not limited to cyclooctynyl or a bicyclononynyl (BCN), difluorinated cyclooctynyl (DIFO), dibenzocyclooctynyl (DIBO), keto-DIBO, biarylazacyclooctynonyl (BARAC), dibenzoazacyclooctynyl (DIBAC, DBCO, ADIBO), dimethoxyazacyclooctynyl (DIMAC), difluorobenzocyclooctynyl (DIFBO), monobenzocyclooctynyl (MOBO), and tetramethoxy dibenzocyclooctynyl (TMDIBO).

In certain embodiments, R₄ is dibenzoazacyclooctynyl (DIBAC, DBCO, ADIBO), which has the following structure:

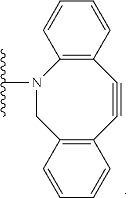 [SMILES: *N1Cc2ccccc2C#Cc2ccccc21]

In embodiments in which R₄ is DBCO, the DBCO can be covalently linked to a compound directly or indirectly via a linker, and is preferably attached to the compound indirectly via a linker.

In certain embodiments, R₄ comprises an antigen binding domain. The antigen binding domain can be linked to the compound directly via a covalent linkage, or indirectly via a linker. In preferred embodiments, the antigen binding domain has binding specificity for hK2, such as the Fab of KL2B30.

In another embodiment, the chelator is directed to a compound of formula (V):

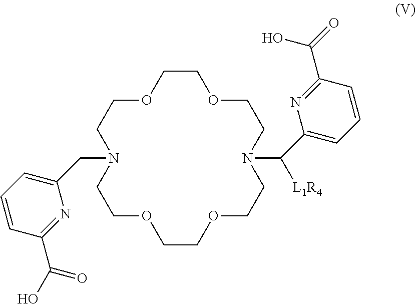

or a pharmaceutically acceptable salt thereof, wherein:
   L₁ is absent or a linker; and
   R₄ is a nucleophilic moiety, an electrophilic moiety, or an antigen binding domain.

In another embodiment the chelator is a compound of formula (VI):

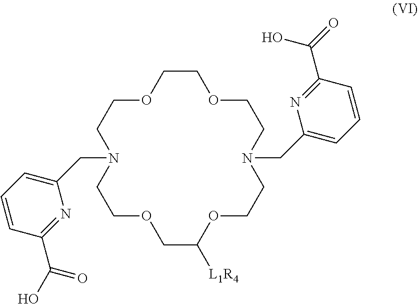

or a pharmaceutically acceptable salt thereof, wherein:
   L₁ is absent or a linker; and
   R₄ is a nucleophilic moiety, an electrophilic moiety, or an antigen binding domain (e.g., the Fab of KL2B30).

In another embodiment, the invention is directed to a compound, wherein: R₁ is -L₁-R₄; R₂ and R₃ are taken together with the carbon atoms to which they are attached to form a 5- or 6-membered cycloalkyl; L₁ is absent or a linker; and R₄ is a nucleophilic moiety, an electrophilic moiety, or an antigen binding domain; or a pharmaceutically acceptable salt thereof.

In a further embodiment, the invention is directed to a compound, wherein R₁ is H; R₂ and R₃ are taken together with the carbon atoms to which they are attached to form a 5- or 6-membered cycloalkyl substituted with -L₁-R₄; L₁ is absent or a linker; and R₄ is a nucleophilic moiety, an electrophilic moiety, or an antigen binding domain; or a pharmaceutically acceptable salt thereof:

Additional embodiments include those wherein R₄ is an antigen binding domain. According to preferred embodiments, R₄ comprises an antigen binding domain with binding specificity for hK2, such as the Fab of KL2B30.
In an embodiment, the chelators are any one or more independently selected from the group consisting of

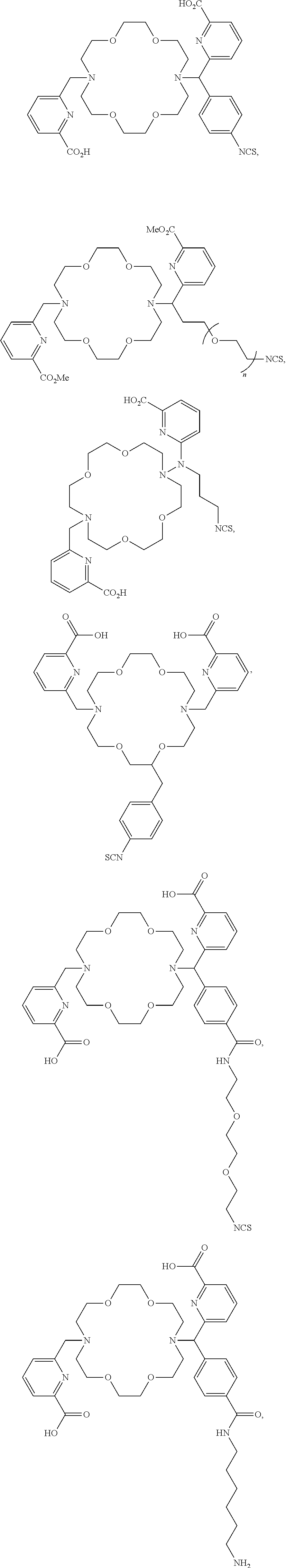

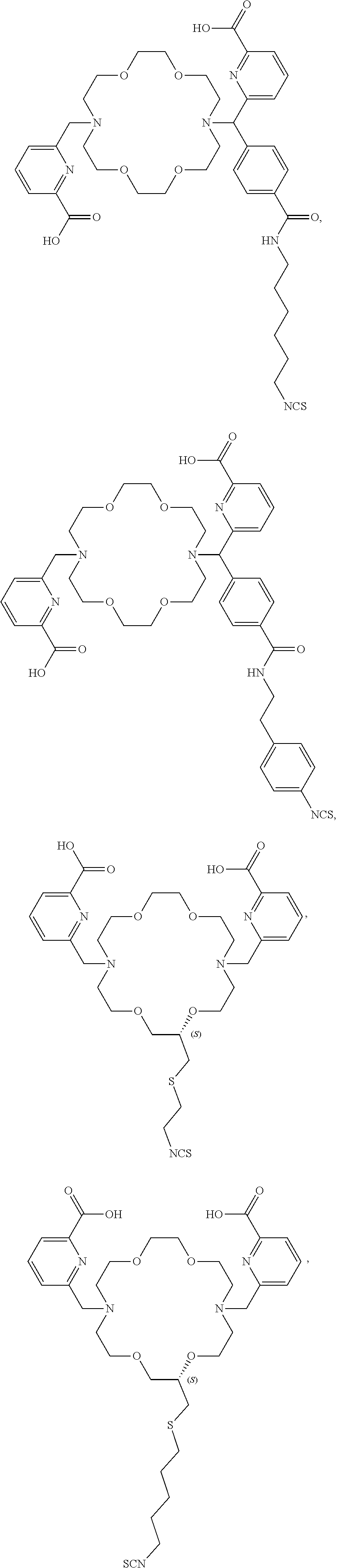

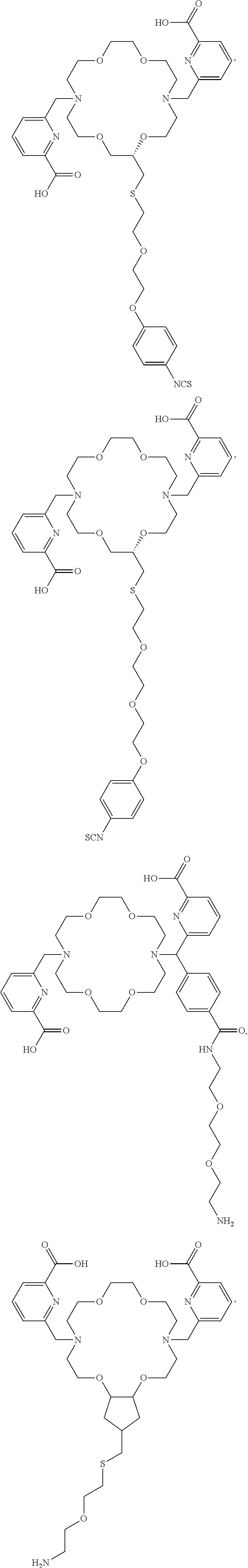

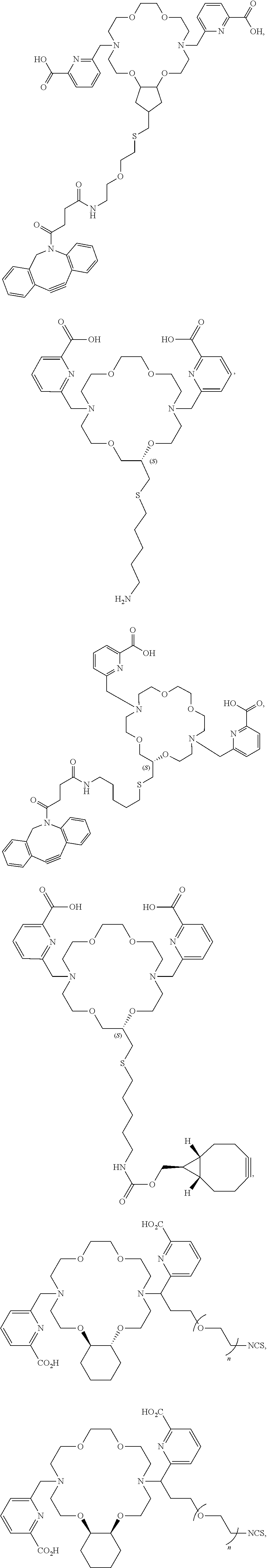

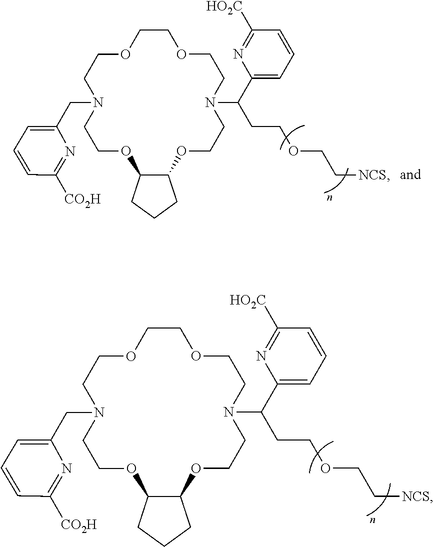

wherein n is 1-10.

Said chelators can be covalently attached to an antigen binding domain (e.g., the Fab of KL2B30) to form immunoconjugates or radioimmunoconjugates by reacting the compound with an azide-labeled antigen binding domain to form a 1,2,3-triazole linker, e.g., via a click chemistry reaction as described in WO2020/229974.

Chelators, radiometal complexes and radioimmunoconjugates of the present invention can be produced by any method known in the art in view of the present disclosure; for example, the pendant aromatic/heteroaromatic groups can be attached to the macrocyclic ring portion by methods known in the art, such as those exemplified and described in WO2020/229974.

### Radiometal Complexes

In another general aspect, the invention relates to a radiometal complex comprising a radiometal ion coordinated to a chelator of the invention via coordinate bonding. Any of the chelators of the invention described herein can comprise a radiometal ion. Preferably, the radiometal ion is an alpha-emitting radiometal ion, more preferably ²²⁵Ac. Chelators of the invention can robustly chelate radiometal ions, particularly ²²⁵Ac at any specific activity irrespective of metal impurities, thus forming a radiometal complex having high chelation stability in vivo and in vitro and which is stable to challenge agents, e.g., diethylene triamine pentaacetic acid (DTPA).

According to embodiments of the invention, a radiometal complex has the structure of formula (I-m):

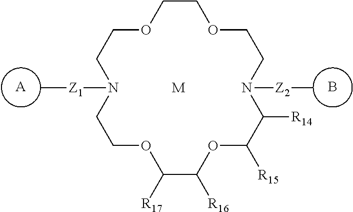

wherein the variable groups are as defined above in the chelators of the invention, e.g., the chelator of formula (I); and M is a radiometal ion. The radiometal ion M is bound to the chelator via coordinate bonding to form the radiometal complex. Heteroatoms of the macrocyclic ring of the chelator as well as any functional groups of the pendant arms (i.e., —Z₁-ring A and/or —Z₂-ring B) can participate in coordinate bonding of the radiometal ion.

Any of the chelators of formula (I) described above can be used to form radiometal complexes of formula (I-m).

In certain embodiments, the radiometal ion M is an alpha-emitting radiometal ion Preferably, the alpha-emitting radiometal ion is ²²⁵Ac.

According to embodiments of the invention, a radiometal complex comprises at least one X group, wherein X is -L₁-R₁₁, wherein L₁ is absent or a linker, and R₁₁ is an electrophilic moiety or a nucleophilic moiety, or R₁₁ comprises an antigen binding domain (e.g., the Fab of KL2B30). When R₁₁ is a nucleophilic or electrophilic moiety, such moiety can be used for attachment of the radiometal complex to an antigen binding domain, directly or indirectly via a linker.

In certain embodiments, a radiometal comprises a single X group, and preferably L₁ of the X group is a linker.

In particular embodiments, R₁ is —NH₂, —NCS (isothiocyanate), —NCO (isocyanate), —N₃ (azido), alkynyl, cycloalkynyl, carboxylic acid, ester, amido, alkylamide, maleimido, acyl halide, tetrazine, or trans-cyclooctene, more particularly —NCS, —NCO, —N₃, alkynyl, cycloalkynyl, —C(O)R₁₃, —COOR₁₃, —CON(R₁₃)₂, maleimido, or acyl halide (e.g., —C(O)Cl or —C(O)Br), wherein each R₁₃ is independently hydrogen or alkyl.

In some embodiments, R₁₁ is an alkynyl, cycloalkynyl, or azido group thus allowing for attachment of the chelator to an antigen binding domain or other chemical moiety (e.g., linker) using a click chemistry reaction.

In certain embodiments, R₁₁ is an alkynyl group, more preferably a terminal alkynyl group or cycloalkynyl group that is reactive with an azido group, particularly via strain-promoted azide-alkyne cycloaddition (SPAAC). Examples of cycloalkynyl groups that can react with azide groups via SPAAC include, but are not limited to cyclooctynyl or a cyclooctynyl derivative, such as bicyclononynyl (BCN), difluorinated cyclooctynyl (DIFO), dibenzocyclooctynyl (DIBO), keto-DIBO, biarylazacyclooctynonyl (BARAC), dibenzoazacyclooctynyl (DIBAC, DBCO, ADIBO), dimethoxyazacyclooctynyl (DIMAC), difluorobenzocyclooctynyl (DIFBO), monobenzocyclooctynyl (MOBO), and tetramethoxy dibenzocyclooctynyl (TMDIBO).

In a particular embodiment, R₁₁ is dibenzoazacyclooctynyl (DIBAC, DBCO, ADIBO), which has the following structure:

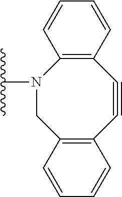 [SMILES: *N1Cc2ccccc2C#Cc2ccccc21]

In such embodiments in which R₁₁ is DBCO, the DBCO can be covalently linked to a radiometal complex directly or indirectly via a linker, and is preferably attached to the radiometal complex indirectly via a linker.

In another particular embodiment, R₁₁ is bicyclononynyl (BCN).

According to embodiments of the invention, each of ring A and ring B is independently a 6-10 membered aryl or a 5-10 membered heteroaryl. In alternative embodiments, it is contemplated that each of ring A and ring B is an optionally substituted heterocyclyl ring, such as oxazoline. Each of ring A and ring B can be optionally and independently substituted with one or more substituent groups independently selected from the group consisting of halo, alkyl, alkenyl, cycloalkyl, cycloalkenyl, aryl, heterocyclyl, heteroaryl, —OR₁₃, —SR₁₃, —(CH₂)ₚCOOR₁₃, —OC(O)R₁₃, —N(R₁₃)₂, —CON(R₁₃)₂, —NO₂, —CN —OC(O)N(R₁₃)₂, and X. Examples of 6-10 membered aryl groups suitable for this purpose include, but are not limited to, phenyl and naphthyl. Examples of 5 to 10 membered heteroaryl groups suitable for this purpose include, but are not limited to pyridinyl, isothiazolyl, isoxazolyl, and imidazolyl. Examples of suitable substituents of the 5 to 10 membered heteroaryl and 6 to 10 membered aryl groups include, but are not limited to —COOH, tetrazolyl, and —CH₂COOH.

In certain embodiments, each of ring A and ring B are independently and optionally substituted with one or more carboxyl groups, including but not limited to, —COOH and —CH₂COOH.

In one embodiment, ring A and ring B are the same, e.g., both ring A and ring B are pyridinyl. In another embodiment, ring A and ring B are different, e.g., one of ring A and ring is pyridinyl and the other is phenyl.

In a particular embodiment, both ring A and ring B are pyridinyl substituted with —COOH.

In a particular embodiment, both ring A and ring B are pyridinyl substituted with tetrazolyl.

In another particular embodiment, both ring A and ring B are picolinic acid groups having the following structure:

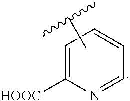

According to embodiments of the invention, each of Z₁ and Z₂ is independently —(C(R₁₂)₂)ₘ— or —(CH₂)ₙ—C(R₁₂)(X)—(CH₂)ₙ—; each X is independently -L₁-R₁₁; each n is independently 0, 1, 2, 3, 4, or 5; and each m is independently 1, 2, 3, 4, or 5.

In some embodiments, both Z₁ and Z₂ are —(CH₂)ₘ—, wherein each m is preferably 1. In such embodiments, a carbon atom of the macrocyclic ring, ring A, or ring B is substituted with an X group.

In some embodiments, one of Z₁ and Z₂ is —(CH₂)ₙ—C(R₁₂)(X)—(CH₂)ₙ— and the other is —(CH₂)ₘ—.

In some embodiments, one of Z₁ and Z₂ —(CH₂)ₙ—C(R₁₂)(X)—(CH₂)ₙ— and the other is —(CH₂)ₘ—; each n is 0; m is 1; X is -L₁-R₁₁; and L₁ is a linker.

In some embodiments, both Z₁ and Z₂ are —(CH₂)ₘ—; each m is independently 0, 1, 2, 3, 4, or 5, preferably each m is 1; and one of R₁₄, R₁₅, R₁₆, and R₁₇ is X, and the rest of R₁₄, R₁₅, R₁₆, and R₁₇ are each hydrogen.

In some embodiments, R₁₄ and R₁₅ are taken together with the carbon atoms to which they are attached to form a 5- or 6-membered cycloalkyl ring (e.g., cyclopentyl or cyclohexyl). Such 5- or 6-membered cycloalkyl ring can be substituted with an X group.

In some embodiments R₁₆ and R₁₇ are taken together with the carbon atoms to which they are attached to form a 5- or 6-membered cycloalkyl ring (e.g., cyclopentyl or cyclohexyl). Such 5- or 6-membered cycloalkyl ring can be substituted with an X group.

In certain embodiments a radiometal complex has the structure of formula (II-m):

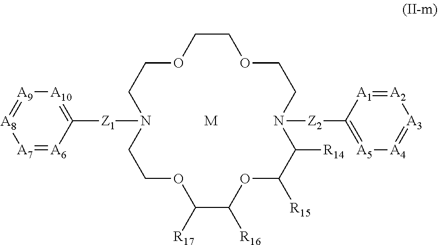

wherein the variable groups are as defined above in the chelators of the invention, e.g., the chelator of formula (II); and M is a radiometal ion, preferably an alpha-emitting radiometal ion, more preferably ²²⁵Ac.

Any of the chelators of formula (II) described above can be used to form radiometal complexes of formula (II-m).

In some embodiments, one of A₁, A₂, A₃, A₄, and A₅ is nitrogen, one of A₁, A₂, A₃, A₄, and A₅ is carbon substituted with —COOH and the rest are CH, i.e., forming a pyridinyl ring substituted with carboxylic acid.

In some embodiments, one of A₆, A₇, A₈, A₉, and A₁₀ is nitrogen, one of A₆, A₇, A₈, A₉, and A₁₀ is carbon substituted with —COOH, and the rest are CH, i.e., forming a pyridinyl ring substituted with carboxylic acid.

In one embodiment, at least one of R₁, R₂, R₃, R₄ and R₅ is —COOH. In one embodiment, at least one of R₆, R₇, R₈, R₉, and R₁₀ is —COOH. In another embodiment, at least one of R₁, R₂, R₃, R₄ and R₅ is —COOH; and at least one of R₆, R₇, R₈, R₉, and R₁₀ is —COOH.

In some embodiments, each of A₁ and A₁₀ is nitrogen; A₂ is CR₂ and R₂ is —COOH; A₉ is CR₉ and R₉ is —COOH; each of A₃-A₈ is CR₂, CR₃, CR₄, CR₅, CR₆, CR₇, and CR₈, respectively; and each of R₃ to R₈ is hydrogen.

In one embodiment, at least one of R₁, R₂, R₃, R₄ and R₅ is tetrazolyl. In one embodiment, at least one of R₆, R₇, R₈, R₉, and R₁₀ is tetrazolyl. In another embodiment, at least one of R₁, R₂, R₃, R₄ and R₅ is tetrazolyl; and at least one of R₆, R₇, R₈, R₉, and R₁₀ is tetrazolyl.

In some embodiments, each R₁₂ is hydrogen.

In some embodiments, R₁₁ is an alkynyl group or cycloalkynyl group, preferably cyclooctynyl or a cyclooctynyl derivative, e.g., DBCO.

In particular embodiments of a radiometal complex of formula (II-m):

M is ²²⁵Ac;

each of A₁ and A₁₀ is nitrogen;

A₂ is CR₂ and R₂ is —COOH;

A₉ is CR₉ and R₉ is —COOH;

each of A₃-A₈ is CR₂, CR₃, CR₄, CR₅, CR₆, CR₇, and CR₈, respectively;

each of R₃ to R₈ is hydrogen;

one of Z₁ and Z₂ is —(CH₂)ₘ— and the other of Z₁ and Z₂ is —(CH₂)ₙ—C(R₁₂)(X)—(CH₂)ₙ—;

R₁₂ is hydrogen;

m is 1;

each n is 0;

X is -L₁-R₁₁, wherein L₁ is a linker and —R₁₁ is an electrophilic group, e.g., cyclooctynyl or cyclooctynyl derivative such as DBCO; and

each of R₁₄-R₁₇ is hydrogen, or alternatively R₁₆ and R₁₇ are taken together with the carbon atoms to which they are attached to form a 5- or 6-membered cycloalkyl ring.

In certain embodiments, a radiometal complex has the structure of formula (III-m):

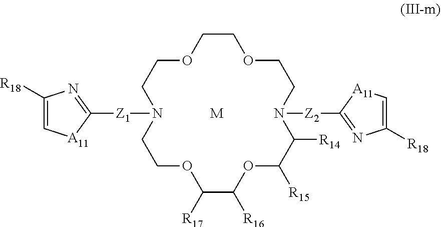

wherein the variable groups are as defined above in the chelators of the invention, e.g., the chelator of formula (III); and M is a radiometal ion, preferably an alpha-emitting radiometal ion, more preferably ²²⁵Ac.

Any of the chelators of formula (III) described above can be used to form radiometal complexes of formula (III-m).

In some embodiments, each A₁₁ is the same, and each A₁₁ is 0, S, NMe, or NH. For example, each A₁₁ can be S. In other embodiments, each A₁₁ is different and each is independently selected from O, S, NMe, and NH.

In some embodiments, each R₁₈ is independently —(CH₂)ₚ—COOR₁₃, wherein R₁₃ is hydrogen and each p is independently 0 or 1.

In some embodiments, each R₁₈ is —COOH.

In some embodiments, each R₁₈ is —CH₂COOH.

In some embodiments, each R₁₈ is tetrazolyl.

In particular embodiments of a radiometal complex of formula (III-m):
each R₁₈ is COOH;
one of Z₁ and Z₂ is —(CH₂)ₘ— and the other of Z₁ and Z₂ is —(CH₂)ₙ—C(R₁₂)(X)—(CH₂)ₙ—;
R₁₂ is hydrogen;
m is 1; each n is 0;
X is -L₁-R₁₁, wherein L₁ is a linker and —R₁₁ is an electrophilic group, e.g., cyclooctynyl or cyclooctynyl derivative such as DBCO; and
each of R₁₄-R₁₇ is hydrogen, or alternatively R₁₆ and R₁₇ are taken together with the carbon atoms to which they are attached to form a 5- or 6-membered cycloalkyl ring.

In particular embodiments of the invention, a radiometal complex has one of the following structures:

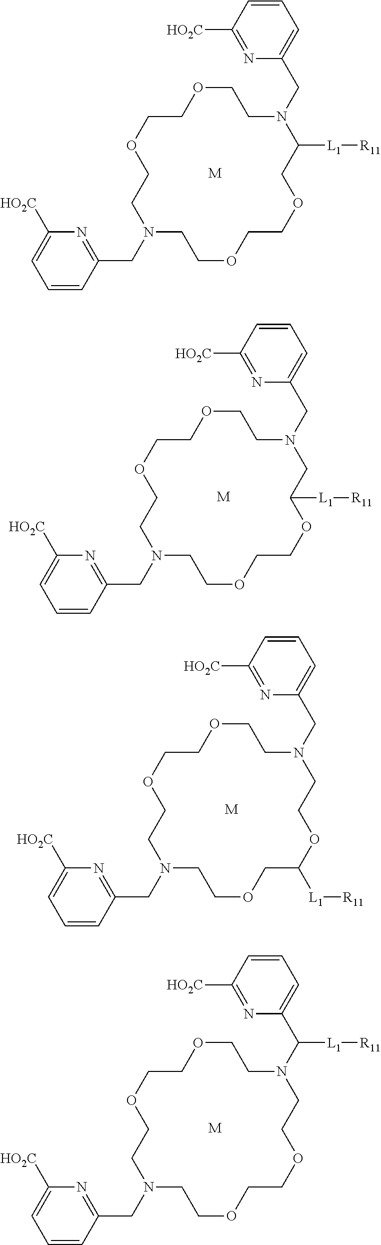

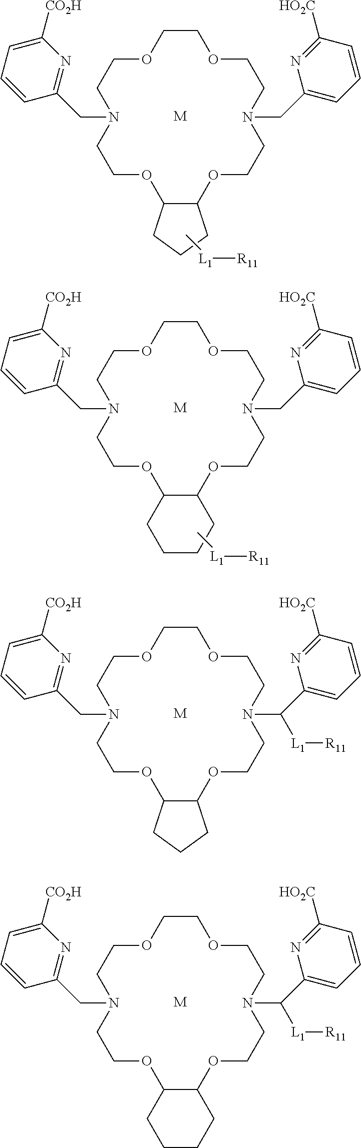

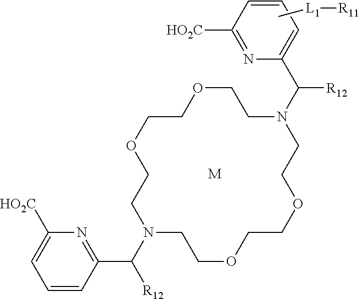

wherein:
M is actinium-225 (²²⁵Ac), L₁ is absent or a linker;
R₁₁ is a nucleophilic moiety or an electrophilic moiety, or R₁ comprises an antigen binding domain (e.g., the Fab of KL2B30); and
each R₁₂ is independently hydrogen, —CH₃, or —CH₂CH₃, provided at least one R₁₂ is —CH₃ or —CH₂CH₃.

In certain embodiments, the invention is directed to a radiometal complex structure of formula (IV-m):

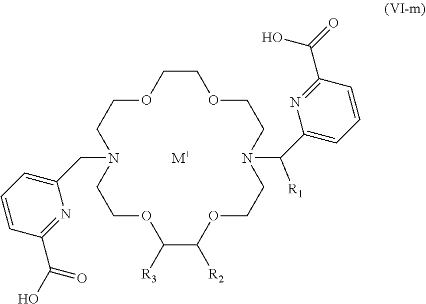

or a pharmaceutically acceptable salt thereof, wherein:
   M⁺ is a radiometal ion, wherein M⁺ is selected from the group consisting of actinium-225(²²⁵Ac), radium-223 (²³³Ra), bismuth-213 (²¹³Bi), lead-212 (²¹²Pb(II) and/or ²¹²Pb(IV)), terbium-149 (¹⁴⁹Tb), terbium-152 (¹⁵²Tb), terbium-155 (¹⁵⁵Tb),fermium-255 (²⁵⁵Fm), thorium-227 (²²⁷Th), thorium-226 (²²⁶Th⁴⁺), astatine-211 (²¹¹At), cerium-134 (¹³⁴Ce), neodymium-144 (¹⁴⁴Nd), lanthanum-132 (¹³²La), lanthanum-135 (¹³⁵La) and uranium-230 (²³⁰U);
   R₁ is hydrogen and R₂ is -L1-R₄;
   alternatively, R₁ is -L1-R₄ and R₂ is hydrogen;
   R₃ is hydrogen;
   alternatively, R₂ and R₃ are taken together with the carbon atoms to which they are attached to form a 5- or 6-membered cycloalkyl, wherein the 5- or 6-membered cycloalkyl is optionally substituted with -L1-R₄;
   L₁ is absent or a linker;
   R₄ is a nucleophilic moiety, an electrophilic moiety, or an antigen binding domain (e.g., the Fab of KL2B30).

In another embodiment, the invention is directed to a radiometal complex of formula (V-m):

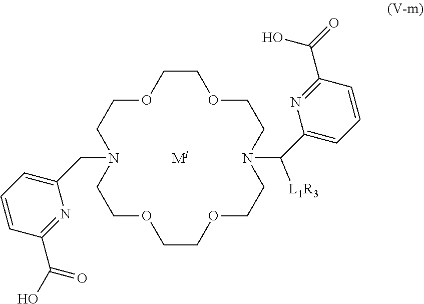

or a pharmaceutically acceptable salt thereof, wherein:
   M⁺ is a radiometal ion, wherein M⁺ is selected from the group consisting of actinium-225(²²⁵Ac), radium-223 (²³³Ra), bismuth-213 (²¹³Bi), lead-212 (²¹²Pb(II) and/or ²¹²Pb(IV)), terbium-149 (¹⁴⁹Tb), terbium-152 (¹⁵²Tb), terbium-155 (¹⁵⁵Tb),fermium-255 (²⁵⁵Fm), thorium-227 (²²⁷Th), thorium-226 (²²⁶Th⁴⁺), astatine-211 (²¹¹At), cerium-134 (¹³⁴Ce), neodymium-144 (¹⁴⁴Nd), lanthanum-132 (¹³²La), lanthanum-135 (¹³⁵La) and uranium-230 (²³⁰U);
   L₁ is absent or a linker;
   R₄ is a nucleophilic moiety, an electrophilic moiety, or an antigen binding domain (e.g., the Fab of KL2B30).

In another embodiment, the invention is directed to a compounds of formula (VI-m):

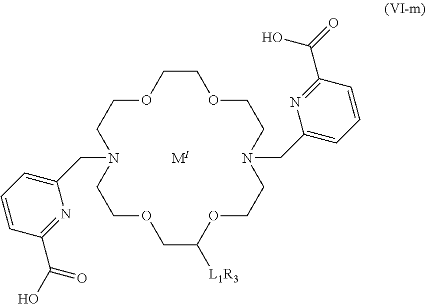

or a pharmaceutically acceptable salt thereof, wherein:

M⁺ is a radiometal ion, wherein M⁺ is selected from the group consisting of actinium-225(²²⁵Ac), radium-223 (²³³Ra), bismuth-213 (²¹³Bi), lead-212 (²¹²Pb(II) and/or ²¹²Pb(IV)), terbium-149 (¹⁴⁹Tb), terbium-152 (¹⁵²Tb), terbium-155 (¹⁵⁵Tb),fermium-255 (²⁵⁵Fm), thorium-227 (²²⁷Th), thorium-226 (²²⁶Th⁴⁺), astatine-211 (²¹¹At), cerium-134 (¹³⁴Ce), neodymium-144 (¹⁴⁴Nd), lanthanum-132 (¹³²La), lanthanum-135 (¹³⁵La) and uranium-230 (²³⁰U); L₁ is absent or a linker; and

R₄ is a nucleophilic moiety, an electrophilic moiety, or an antigen binding domain (e.g., the Fab of KL2B30).

In another embodiment, the invention is directed to a is radiometal complex

wherein:
M⁺ is a radiometal ion, wherein M⁺ is selected from the group consisting of actinium-225(²²⁵Ac), radium-223 (²³³Ra), bismuth-213 (²¹³Bi), lead-212 (²¹²Pb(II) and/or ²¹²Pb(IV)), terbium-149 (¹⁴⁹Tb), terbium-152 (¹⁵²Tb), terbium-155 (¹⁵⁵Tb),fermium-255 (²⁵⁵Fm), thorium-227 (²²⁷Th), thorium-226 (²²⁶Th⁴⁺), astatine-211 (²¹¹At), cerium-134 (¹³⁴Ce), neodymium-144 (¹⁴⁴Nd), lanthanum-132 (¹³²La), lanthanum-135 (¹³⁵La) and uranium-230 (²³⁰U); R₁ is -L₁-R₄;
R₂ and R₃ are taken together with the carbon atoms to which they are attached to form a 5- or 6-membered cycloalkyl;
L₁ is absent or a linker; and
R₄ is a nucleophilic moiety, an electrophilic moiety, or an antigen binding domain (e.g., the Fab of KL2B30);

or a pharmaceutically acceptable salt thereof.

In a further embodiment, the invention is directed to a radiometal complex
wherein
   M⁺ is a radiometal ion, wherein M⁺ is selected from the group consisting of actinium-225(²²⁵Ac), radium-223 (²³³Ra), bismuth-213 (²¹³Bi), lead-212 (²¹²Pb(II) and/or ²¹²Pb(IV)), terbium-149 (¹⁴⁹Tb), terbium-152 (¹⁵²Tb), terbium-155 (¹⁵⁵Tb),fermium-255 (²⁵⁵Fm), thorium-227 (²²⁷Th), thorium-226 (²²⁶Th⁴⁺), astatine-211 (²¹¹At), cerium-134 (¹³⁴Ce), neodymium-144 (¹⁴⁴Nd), lanthanum-132 (¹³²La), lanthanum-135 (¹³⁵La) and uranium-230 (²³⁰U); R₁ is H;
   R₂ and R₃ are taken together with the carbon atoms to which they are attached to form a 5- or 6-membered cycloalkyl substituted with -L₁-R₄;
   L₁ is absent or a linker; and
   R₄ is a nucleophilic moiety, an electrophilic moiety, or an antigen binding domain (e.g., the Fab of KL2B30);
or a pharmaceutically acceptable salt thereof.

In certain embodiments, the invention is directed to any one or more radiometal complexes selected from the group consisting of:

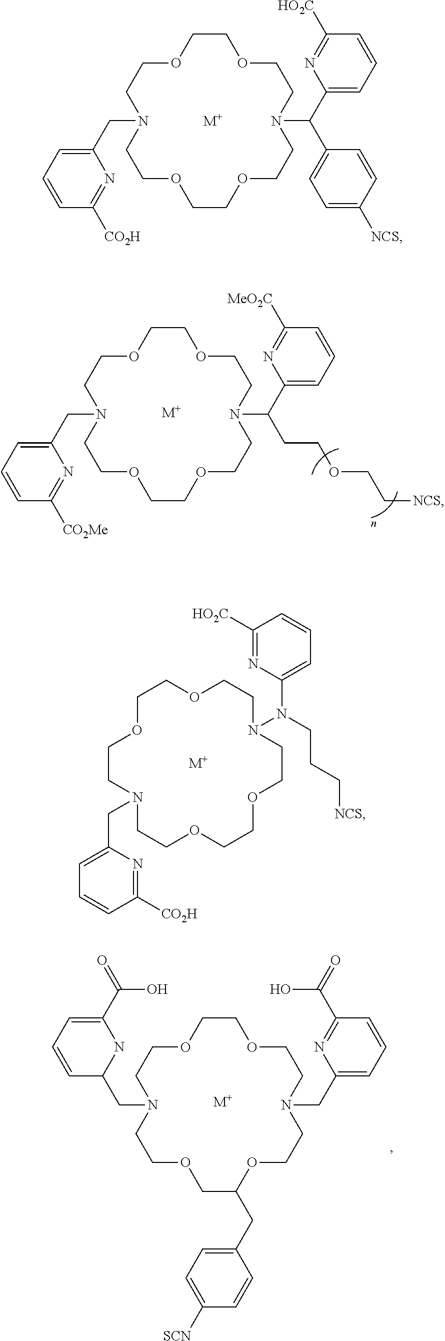

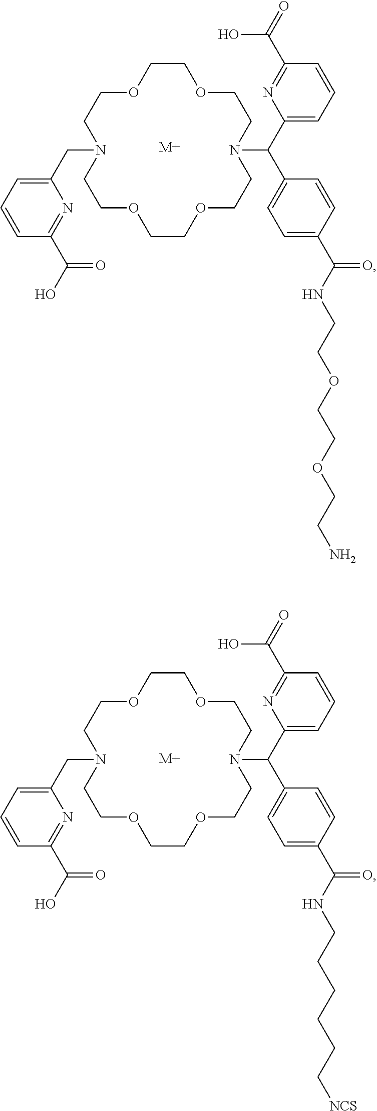

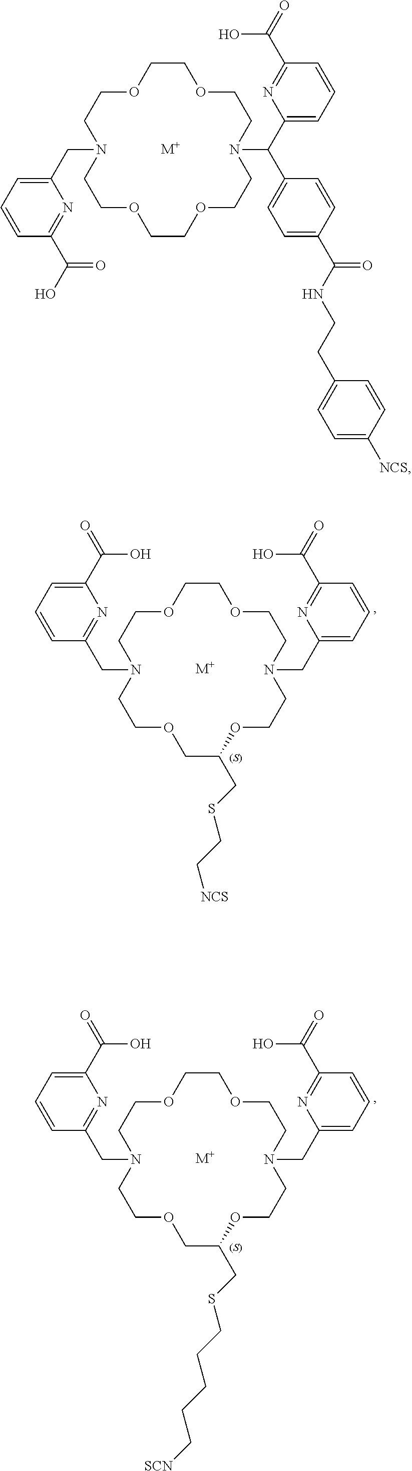

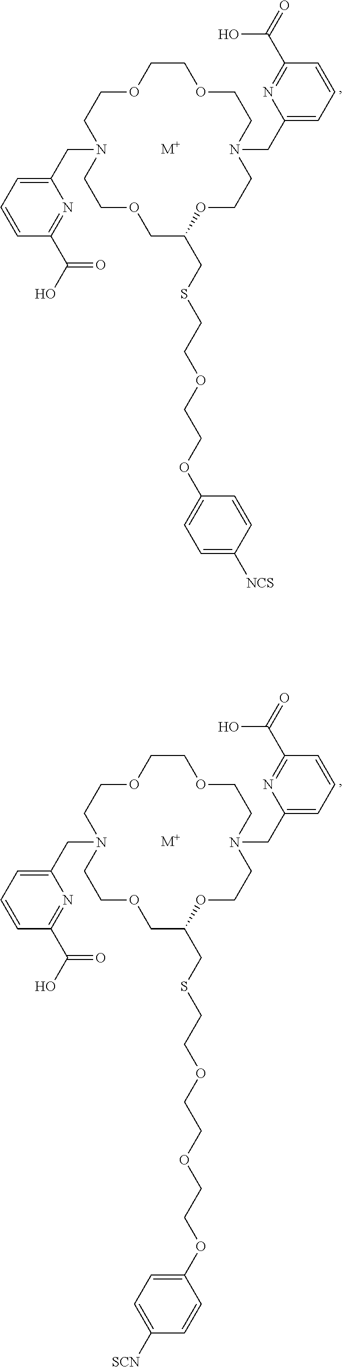

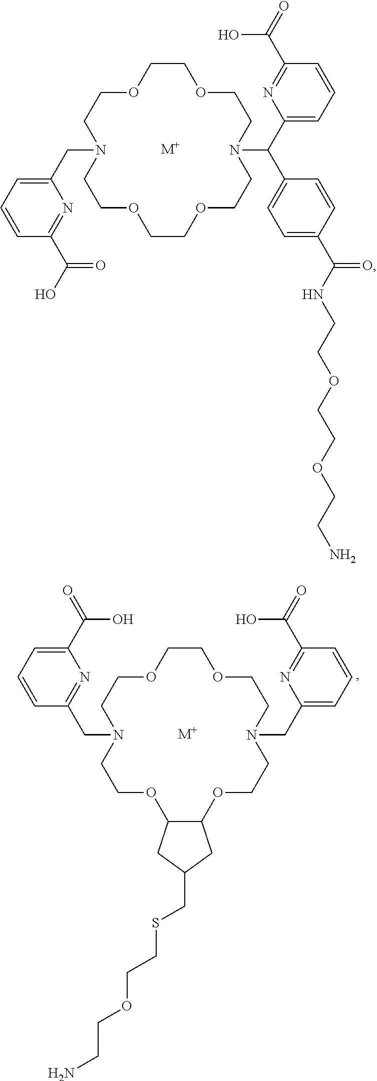

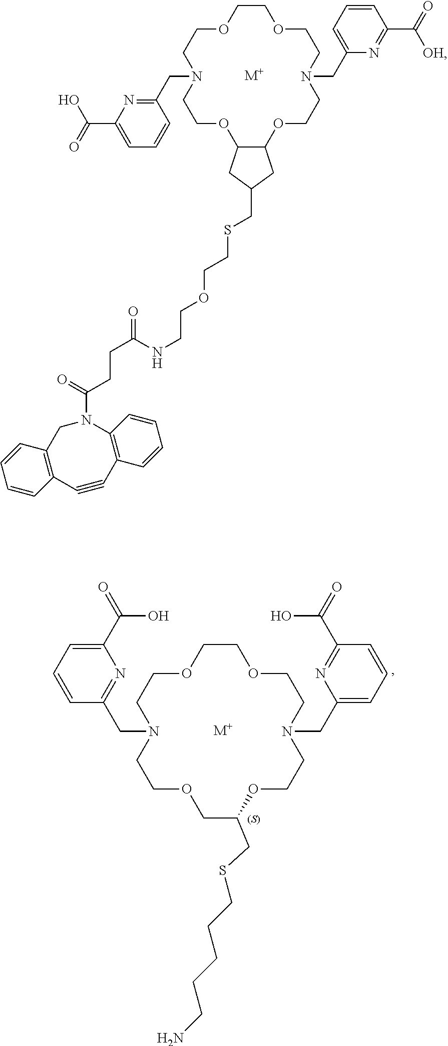

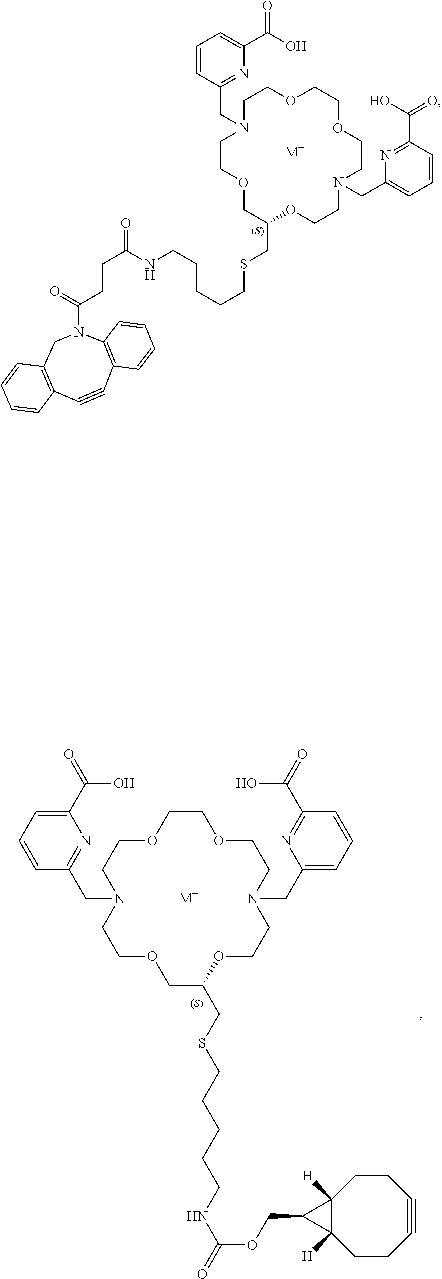

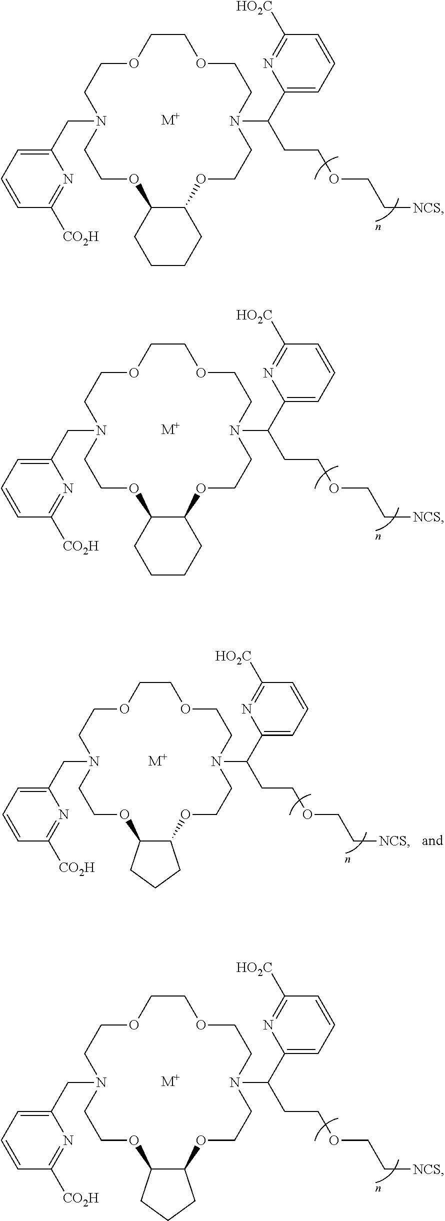

wherein n is 1-10 and M⁺ is a radiometal ion, wherein M⁺ is selected from the group consisting of actinium-225(²²⁵Ac), radium-223 (²³³Ra), bismuth-213 (²¹³Bi), lead-212 (²¹²Pb(II) and/or ²¹²Pb(IV)), terbium-149 (¹⁴⁹Tb), terbium-152 (¹⁵²Tb), terbium-155 (¹⁵⁵Tb),fermium-255 (²⁵⁵Fm), thorium-227 (²²⁷Th), thorium-226 (²²⁶Th⁴⁺), astatine-211 (²¹¹At), cerium-134 (¹³⁴Ce), neodymium-144 (¹⁴⁴Nd), lanthanum-132 (¹³²La), lanthanum-135 (¹³⁵La) and uranium-230 (²³⁰U).

Radiometal complexes can be produced by any method known in the art in view of the present disclosure. For example, a chelator of the invention can be mixed with a radiometal ion and the mixture incubated to allow for formation of the radiometal complex. In an exemplary embodiment, a chelator is mixed with a solution of ²²⁵Ac(NO₃)₃ to form a radiocomplex comprising ²²⁵Ac bound to the chelator via coordinate bonding. As described above, chelators of in the invention efficiently chelate radiometals, particularly ²²⁵Ac. Thus, in particular embodiments, a chelator of the invention is mixed with a solution of ²²⁵Ac ion at a ratio by concentration of chelator to ²²⁵Ac ion of 1:1000, 1:500, 1:400, 1:300, 1:200, 1:100, 1:50, 1:10, or 1:5, preferably 1:5 to 1:200, more preferably 1:5 to 1:100. Thus, in some embodiments, the ratio of a chelator of the invention to ²²⁵Ac which can be used to form a radiometal complex is much lower than that which can be achieved with other known ²²⁵Ac chelators, e.g., DOTA. The radiocomplex can be characterized by instant thin layer chromatography (e.g., iTLC-SG), HPLC, LC-MS, etc. Exemplary methods are described, for example, in WO2020/229974.

### Additional Embodiments of Immunoconjugates and Radioimmunoconjugates

As described herein, chelators and radiometal complexes of the invention can be conjugated to (i.e., covalently linked to) antigen binding domains, such as an immune substance to produce immunoconjugates and/or radioimmunoconjugates that are suitable, for example, for medicinal applications in subjects, e.g., humans, such as targeted radiotherapy. Using the chelators and radiometal complexes of the invention, antigen binding domains, particularly antibodies or antigen binding fragments thereof that can bind specifically to targets of interest (such as cancer cells), can be site-specifically labeled with radiometal ions to produce radioimmunoconjugates. In particular, using the chelators and/or radiometal complexes of the invention, radioimmunoconjugates having high yield chelation of radiometal ions, particularly ²²⁵Ac, and desired chelator-antibody ratio (CAR) can be produced. According to particular embodiments, methods of the present invention provide an average CAR of less than 10, less than 8, less than 6, or less than 4; or a CAR of between about 2 to about 8, or about 2 to about 6, or about 2 to about 4, or about 2 to about 3; or a CAR of about 2, or about 3, or about 4, or about 5, or about 6, or about 7, or about 8.

According to embodiments of the invention, an immunoconjugate comprises a chelator of the invention, e.g., a chelator of formula (I), formula (II), or formula (III) as described herein, covalently linked to an antibody or antigen binding fragment thereof (e.g., the Fab of KL2B30), preferably via a linker. Numerous modes of attachment with different linkages between the chelator and antibody or antigen binding fragment thereof are possible depending on the reactive functional groups (i.e., nucleophiles and electrophiles) on the chelator and antibody or antigen binding fragment thereof.

According to embodiments of the invention, a radioimmunoconjugate comprises a radiometal complex of the invention, e.g., a radiometal complex of formula (I-m), formula (II-m), or formula (III-m) as described herein, covalently linked to an antibody or antigen binding fragment thereof (e.g., the Fab of KL2B30), preferably via a linker.

Any of the chelators or radiometal complexes of the invention, such as those described herein, can be used to produce immunoconjugates or radioimmunoconjugates of the invention.

In some embodiments, a radiometal complex of a radioimmunoconjugate of the invention comprises an alpha-emitting radiometal ion coordinated to the chelator moiety of the radiocomplex. Preferably, the alpha-emitting radiometal ion is ²²⁵Ac.

In particular embodiments, an antibody or antigen binding fragment thereof is linked to a radiocomplex via a triazole moiety to form a radioimmunoconjugate of the invention.

In particular embodiments, the antibody or antigen binding fragment in an immunoconjugate or radioimmunoconjugate of the application can bind specifically to a tumor antigen. Preferably, the antibody or antigen binding fragment binds specifically to hK2.

Immunoconjugates and radioimmunoconjugates of the invention can be prepared by any method known in the art in view of the present disclosure for conjugating ligands, e.g., antibodies, to chelators, including chemical and/or enzymatic methods. For example, immunoconjugates and radioimmunoconjugates can be prepared by a coupling reaction, including by not limited to, formation of esters, thioesters, or amides from activated acids or acyl halides; nucleophilic displacement reactions (e.g., such as nucleophilic displacement of a halide ring or ring opening of a strained ring system); azide-alkyne Huisgen cycloaddition (e.g., 1,3-dipolar cycloaddition between an azide and alkyne to form a 1,2,3-triazole linker); thiolyne addition; imine formation; Diels-Alder reactions between tetrazines and trans-cycloctene (TCO); and Michael additions (e.g., maleimide addition). Numerous other modes of attachment, with different linkages, are possible depending on the reactive functional group used. The attachment of a ligand can be performed on a chelator that is coordinated to a radiometal ion, or on a chelator which is not coordinated to a radiometal ion.

According to an embodiment, a radioimmunoconjugate can be produced by covalently linking a radiometal complex of the invention to an antibody or antigen binding fragment thereof by, for example, a click chemistry reaction. Alternatively, a radioimmunoconjugate can be produced by first preparing an immunoconjugate of the invention by covalently linking a chelator of the invention to an antibody or antigen-binding fragment thereof by, for example, a click chemistry reaction; the immunoconjugate can subsequently be labeled with a radiometal ion to produce a radioimmunoconjugate (referred to as “one-step direct radiolabeling”). Both residue-specific and site-specific methods of conjugation can be used to produce immunoconjugate and radioimmunoconjugates of the invention. Such methods are described, for example, in WO2020/229974.

According to embodiments of the invention, a method of producing a radioimmunoconjugate comprises reacting a chelator or radiocomplex of the invention, wherein R₁₁ is a nucleophilic or electrophilic moiety, with an antibody or antigen binding fragment thereof (e.g., the Fab of KL2B30), or a modified antibody or antigen binding fragment thereof comprising a nucleophilic or electrophilic moiety.

In one embodiment, a method comprises reacting a chelator of the invention with an antibody or antigen binding fragment thereof, or a modified antibody or antigen binding fragment thereof comprising a nucleophilic or electrophilic functional group, to form an immunoconjugate having a covalent linkage between the chelator and antibody or antigen binding fragment thereof, or modified antibody or antigen binding fragment thereof, and reacting the immunoconjugate with a radiometal ion such that the radiometal ion binds the chelator of the immunoconjugate via coordinate binding, thereby forming the radioimmunoconjugate. This embodiment may be referred to as a “one-step direct radiolabeling” method because there is only one chemical reaction step involving the radiometal.

In another embodiment, a method comprises reacting a radiocomplex of the invention with an antibody or antigen binding fragment thereof, or a modified antibody or antigen binding fragment thereof comprising a nucleophilic or electrophilic functional group, thereby forming the radioimmunoconjugate. This embodiment may be referred to as a “click radiolabeling” method. A modified antibody or antigen binding fragment thereof can be produced by any method known in the art in view of the present disclosure, e.g., by labeling an antibody at a particular residue with a biorthogonal reactive functional group using one or more of the above described methods, or by site-specifically incorporating an unnatural amino acid (e.g., azido- or alkynyl-amino acid) into an antibody using one or more of the above described methods. The degree of labeling (DOL), sometimes called degree of substitution (DOS), is a particularly useful parameter for characterizing and optimizing bioconjugates, such as antibody modified by unnatural amino acid. It is expressed as an average number of the unnatural amino acid coupled to a protein molecule (e.g. an antibody), or as a molar ratio in the form of label/protein. The DOL can be determined from the absorption spectrum of the labeled antibody by any known method in the filed.

In certain embodiments, as described herein, immunoconjugates and radioimmunoconjugates of the invention are prepared using a click chemistry reaction. For example, radioimmunoconjugates of the invention can be prepared using a click chemistry reaction referred to as “click radiolabeling”. Click radiolabeling uses click chemistry reaction partners, preferably an azide and alkyne (e.g., cyclooctyne or cyclooctyne derivative) to form a covalent triazole linkage between the radiocomplex (radiometal ion bound to the chelator) and antibody or antigen binding fragment thereof. Click radiolabeling methods of antibodies are described in, e.g., International Patent Application No. PCT/US18/65913, entitled “Radiolabeling of Polypeptides” of which the relevant description is incorporated herein by reference. In other embodiments referred to as “one-step direct radiolabeling,” an immunoconjugate is prepared using a click chemistry reaction between an antibody or antigen binding fragment thereof and a chelator; the immunoconjugate is then contacted with a radiometal ion to form the radioimmunoconjugate.

According to an embodiment, a method of preparing a radioimmunoconjugate comprises binding a radiometal ion to a chelator of the invention (e.g., via coordinate bonding).

An embodiment of the “one-step direct radiolabeling” method may be described as a method of preparing a radioimmunoconjugate comprising: contacting an immunoconjugate (i.e., polypeptide-chelator complex) with a radiometal ion to thereby form the radioimmunoconjugate, wherein the immunoconjugate comprises a chelator of the present invention. According to particular embodiments, the immunoconjugate has been formed via a click chemistry reaction between the chelator of the present invention and the polypeptide. According to particular embodiments, the radioimmunoconjugate has been formed without metal-free conditions (e.g., without any step(s) of removing or actively excluding common metal impurities from the reaction mixture). This is contrary to certain conventional methods in which it is necessary to radiolabel an antibody under strict metal-free conditions to avoid competitive (non-productive) chelation of common metals such as iron, zinc and copper, which introduce significant challenges into the production process.

In a particular embodiment, a method of preparing a radioimmunoconjugate of the invention comprises a “one-step direct radiolabeling” method comprising:
(i) providing a modified polypeptide comprising the polypeptide (e.g., antibody or antigen binding fragment thereof) covalently linked to a first click reaction partner (e.g., an azido group);
(ii) providing a chelator complex comprising a chelator of the present invention covalently linked to a second click reaction partner (e.g., an alkynyl group or cycloalkynyl group);
(iii) contacting the modified polypeptide with the chelator complex under a condition to allow the first click reaction partner (e.g., azido group) to react with the second click reaction partner (e.g., alkynyl group or cycloalkynyl group) to thereby form a polypeptide-chelator complex (i.e., immunoconjugate); and
(iv) contacting the polypeptide-chelator complex with a radiometal ion to thereby prepare the radioimmunoconjugate (wherein the radioimmunoconjugate comprises the polypeptide labeled with the radiometal ion, e.g., a modified antibody or antigen binding fragment thereof labeled with an alpha-emitting radiometal ion bound to the chelator via coordinate bonding).

According to particular embodiments, step (iv) is performed without metal-free conditions.

In an alternative embodiment, a method of preparing a radioimmunoconjugate comprises a “click radiolabeling” method comprising:
(i) providing a modified antibody or antigen binding fragment thereof comprising the antibody or antigen binding fragment thereof covalently linked to an azido group;
(ii) providing a radiocomplex comprising an alpha-emitting radiometal ion bound to a chelator via coordinate bonding, wherein the chelator is covalently linked to an alkynyl group or cycloalkynyl group; and
(iii) contacting the modified antibody or antigen binding fragment thereof with the radiocomplex under a condition to allow the azido group to react with the alkynyl group or cycloalkynyl group to thereby prepare the radioimmunoconjugate.

Conditions for carrying out click chemistry reactions are known in the art, and any conditions for carrying out click chemistry reactions known to those skilled in the art in view of the present disclosure can be used in the invention. Examples of conditions include, but are not limited to, incubating the modified polypeptide and the radiocomplex at a ratio of 1:1 to 1000:1 at a pH of 4 to 10 and a temperature of 20° C. to 70° C.

The click radiolabeling methods described above allow for chelation of the radiometal ion under low or high pH and/or high temperature conditions to maximize efficiency, which can be accomplished without the risk of inactivating the alkyne reaction partner. The efficient chelation and efficient SPAAC reaction between an azide-labeled antibody or antigen binding fragment thereof and the radiocomplex allows radioimmunoconjugates to be produced with high radiochemical yield even with low azide: antibody ratios. The only step in which trace metals must be excluded is the radiometal ion chelation to the chelating moiety; the antibody production, purification, and conjugation steps do not need to be conducted under metal free conditions.

Chelators and radiometal complexes of the invention can also be used in the production of site-specific radiolabeled polypeptides, e.g., antibodies. The click radiolabeling methods described herein facilitate site-specific production of radioimmunoconjugates by taking advantage of established methods to install azide groups site-specifically on antibodies (Li, X., et al. Preparation of well-defined antibody-drug conjugates through glycan remodeling and strain-promoted azide-alkyne cycloadditions. Angew Chem Int Ed Engl, 2014. 53(28): p. 7179-82; Xiao, H., et al., Genetic incorporation of multiple unnatural amino acids into proteins in mammalian cells. Angew Chem Int Ed Engl, 2013. 52(52): p. 14080-3). Methods of attaching molecules to proteins or antibodies in a site-specific manner are known in the art, and any method of site-specifically labeling an antibody known to those skilled in the art can be used in the invention in view of the present disclosure. Examples of methods to site-specifically modify antibodies suitable for use in the invention include, but are not limited to, incorporation of engineered cysteine residues (e.g., THIOMAB™), use of non-natural amino acids or glycans (e.g., seleno cysteine, p-AcPhe, formylglycine generating enzyme (FGE, SMARTag™), etc.), and enzymatic methods (e.g., use of glycotransferase, endoglycosidase, microbial or bacterial transglutaminase (MTG or BTG), sortase A, etc.).

In some embodiments, a modified antibody or antigen binding fragment thereof for use in producing an immunoconjugate or radioimmunoconjugate of the invention is obtained by trimming the antibody or antigen binding fragment thereof with a bacterial endoglycosidase specific for the β-1,4 linkage between a core GlcNac residue in an Fc-glycosylation site of the antibody, such as GlycINATOR (Genovis), which leaves the inner most GlcNAc intact on the Fc, allowing for the site-specific incorporation of azido sugars at that site. The trimmed antibody or antigen binding fragment thereof can then be reacted with an azide-labeled sugar, such as UDP-N-azidoacetylgalactosamine (UDP-GalNAz) or UDP-6-azido 6-deoxy GalNAc, in the presence of a sugar transferase, such as GalT galactosyltransferase or GalNAc transferase, to thereby obtain the modified antibody or antigen binding fragment thereof.

In other embodiments, a modified antibody or antigen binding fragment thereof for use in producing an immunoconjugate or radioimmunoconjugate of the invention is obtained by deglycosylating the antibody or antigen binding fragment thereof with an amidase. The resulting deglycosylated antibody or antigen binding fragment thereof can then be reacted with an azido amine, preferably 3-azido propylamine, 6-azido hexylamine, or any azido-linker-amine or any azido-alkyl/heteroalkyl-amine, such as an azido-polyethylene glycol (PEG)-amine, for example, O-(2-aminoethyl)-O′-(2-azidoethyl)tetraethylene glycol, O-(2-aminoethyl)-O′-(2-azidoethyl)pentaethylene glycol, O-(2-aminoethyl)-O′-(2-azidoethyl)triethylene glycol, etc., or in the presence of a microbial transglutaminase to thereby obtain the modified antibody or antigen binding fragment thereof.

Any radiometal complex described herein can be used to produce a radioimmunoconjugate of the invention. In particular embodiments, the radiometal complex has the structure of formula (I-m), formula (II-m), or formula (III-m). In particular embodiments, the radiometal complex has a structure selected from the group consisting of:

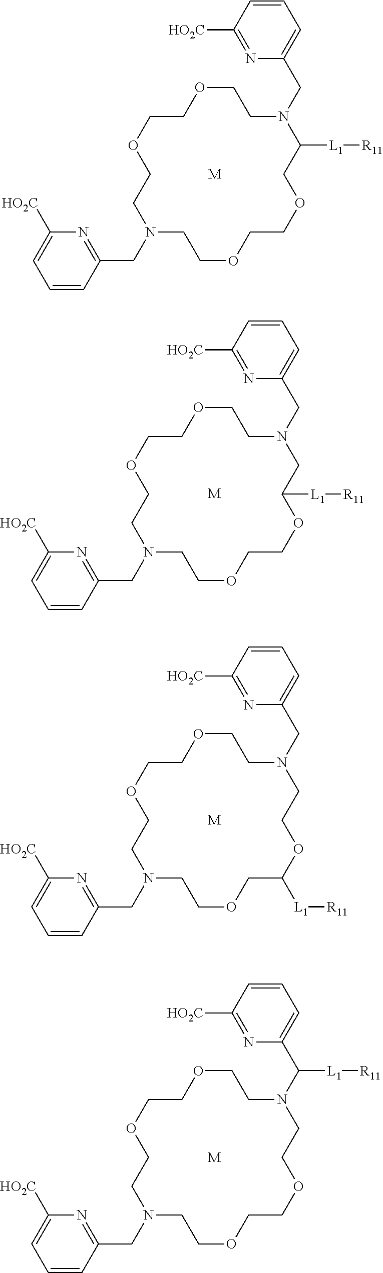

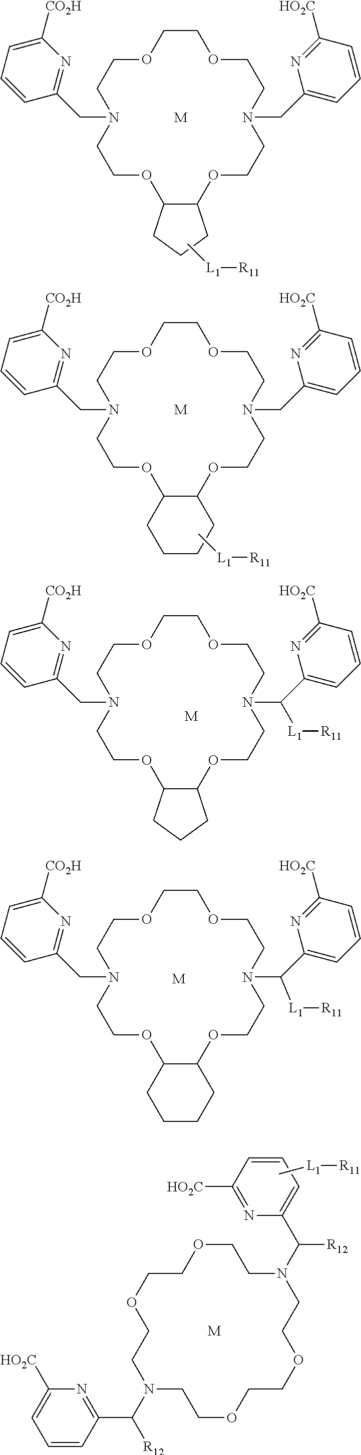

wherein M is a radiometal ion, preferably an alpha-emitting radiometal ion, more preferably actinium-225 (²²⁵Ac), R₁₁ is cyclooctynyl or a cyclooctynyl derivative, such as bicyclononynyl (BCN), difluorinated cyclooctynyl (DIFO), dibenzocyclooctynyl (DIBO), keto-DIBO, biarylazacyclooctynonyl (BARAC), dibenzoazacyclooctynyl (DIBAC, DBCO, ADIBO), dimethoxyazacyclooctynyl (DIMTAC), difluorobenzocyclooctynyl (DIFBO), monobenzocyclooctynyl (MOBO), and tetramethoxy dibenzocyclooctynyl (TMDIBO).

In some embodiments, an antibody or antigen binding fragment thereof is covalently linked to an azido group using any method for chemical or enzymatic modification of antibodies and polypeptides known to those skilled in the art in view of the present disclosure. The azido-labeled antibody or antigen binding fragment thereof is reacted with a chelator or radiometal complex of the invention comprising an alkynyl or cycloalkynyl group, preferably a cyclooctynyl group and more preferably DBCO under conditions sufficient for the azido and alkynyl or cycloalkynyl group to undergo a click chemistry reaction to form a 1,2,3-triazole moiety.

In particular embodiments, the radioimmunoconjugates of the application include, but are not limited to:

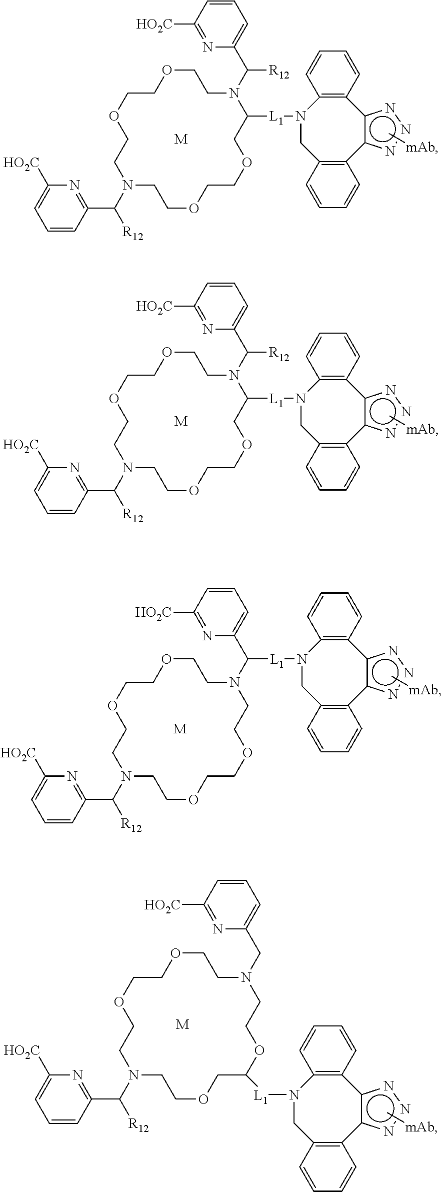

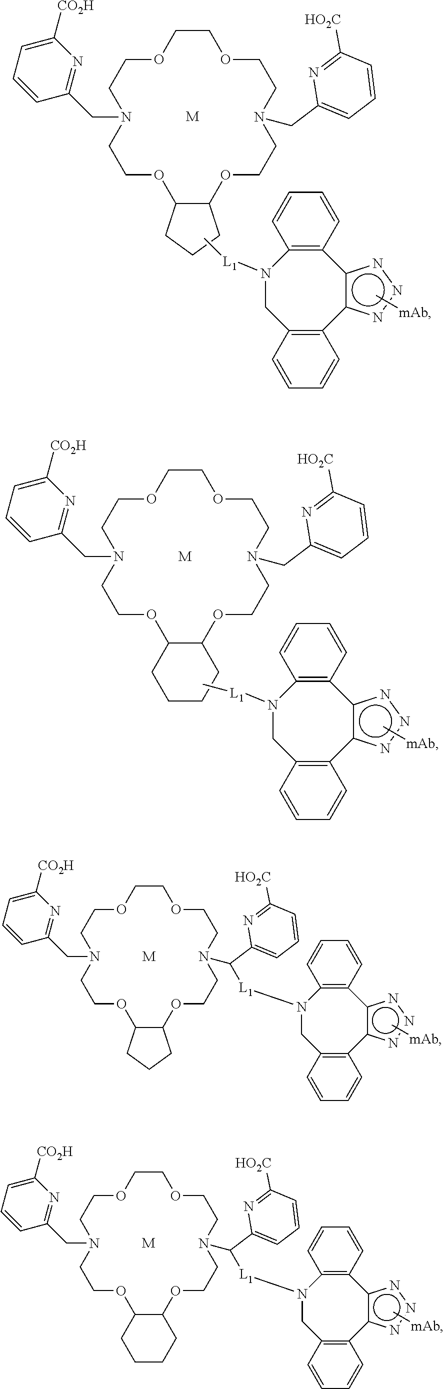

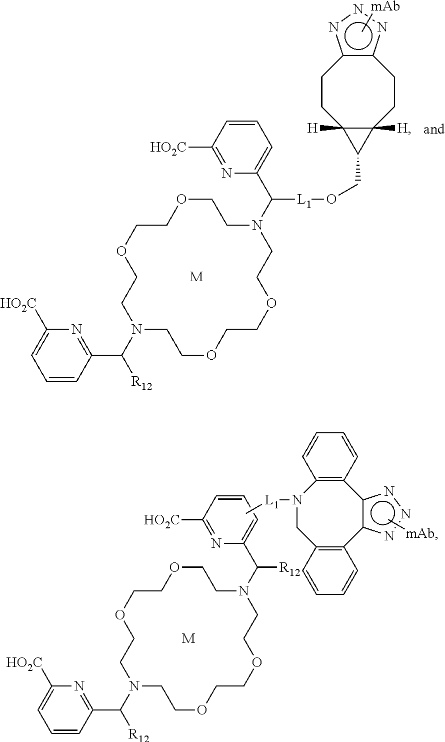

wherein “mAb” is an antibody or antigen binding domain (e.g., the Fab of KL2B30); L₁ is absent or a linker, preferably a linker; each R₁₂ is independently hydrogen, CH₃ or CH₂CH₃, provided at least one R₁₂ is —CH₃ or —CH₂CH₃; and M is an alpha-emitting radionuclide, preferably ²²⁵Ac.

Examples of radioimmunoconjugates of the application include, but are not limited to:

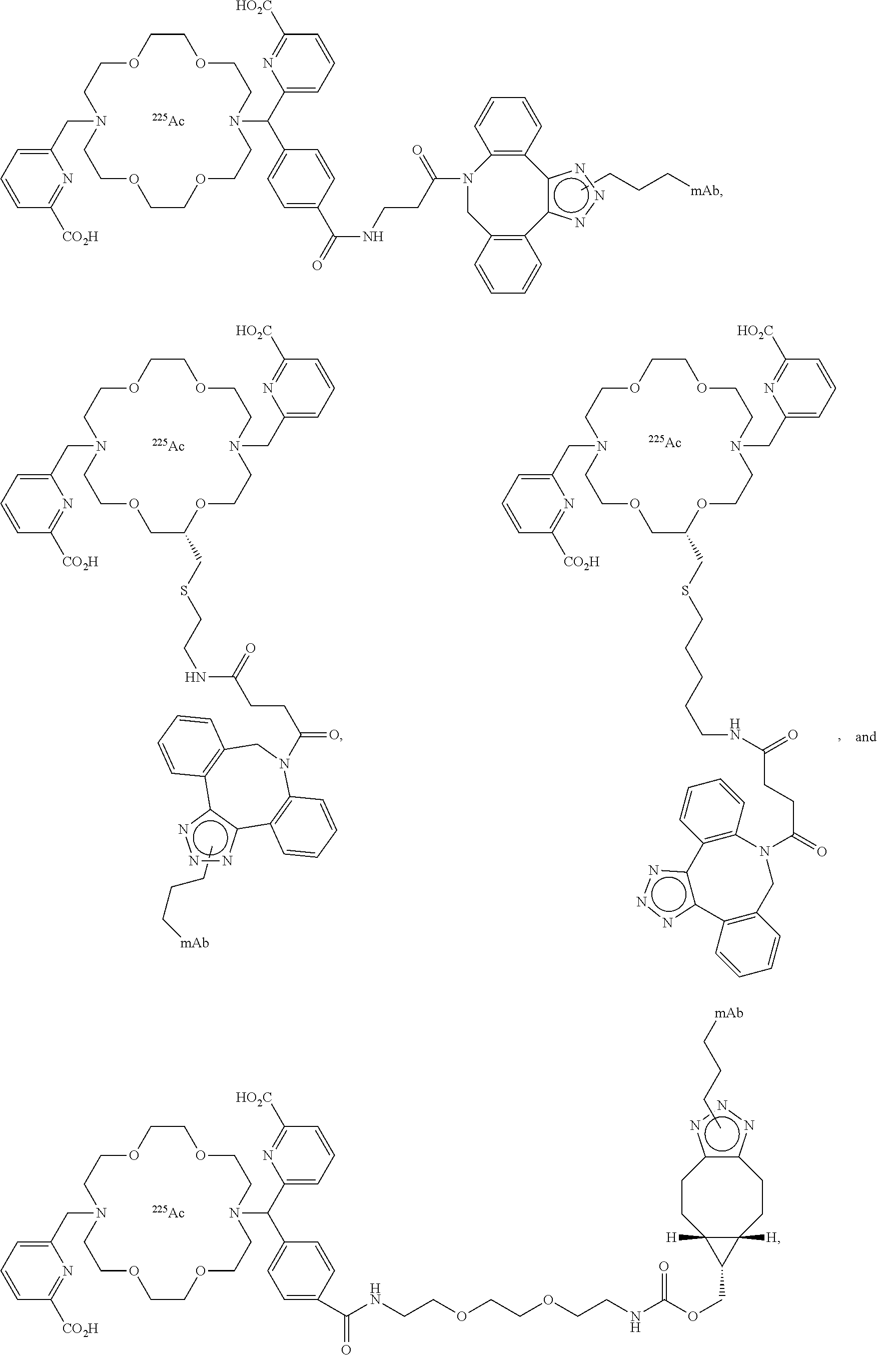

wherein “mAb” preferably refers to an antibody or antigen binding domain that has binding specificity for hK2, such as the Fab of KL2B30, or otherwise described herein.

In certain embodiments, the radioimmunoconjugate is any one or more structures independently selected from the group consisting of:

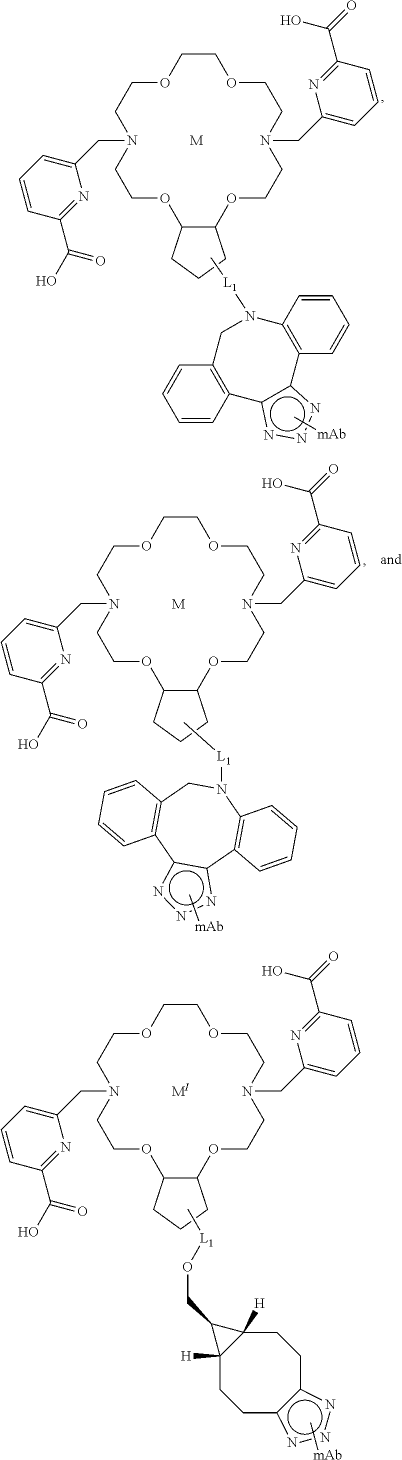

wherein:
   M⁺ is a radiometal ion, wherein M⁺ is selected from the group consisting of actinium-225(²²⁵Ac), radium-223 (²³³Ra), bismuth-213 (²¹³Bi), lead-212 (²¹²Pb(II) and/or ²¹²Pb(IV)), terbium-149 (¹⁴⁹Tb), terbium-152 (¹⁵²Tb), terbium-155 (¹⁵⁵Tb),fermium-255 (²⁵⁵Fm), thorium-227 (²²⁷Th), thorium-226 (²²⁶Th⁴⁺), astatine-211 (²¹¹At), cerium-134 (¹³⁴Ce), neodymium-144 (¹⁴⁴Nd), lanthanum-132 (¹³²La), lanthanum-135 (¹³⁵La) and uranium-230 (²³⁰U);
   L₁ is absent or a linker; and
   wherein “mAb” preferably refers to an antibody or antigen binding domain that has binding specificity for hK2, such as the Fab of KL2B30, or otherwise described herein.
   In another embodiment, the radioimmunoconjugate is any one or more selected from the group consisting of:

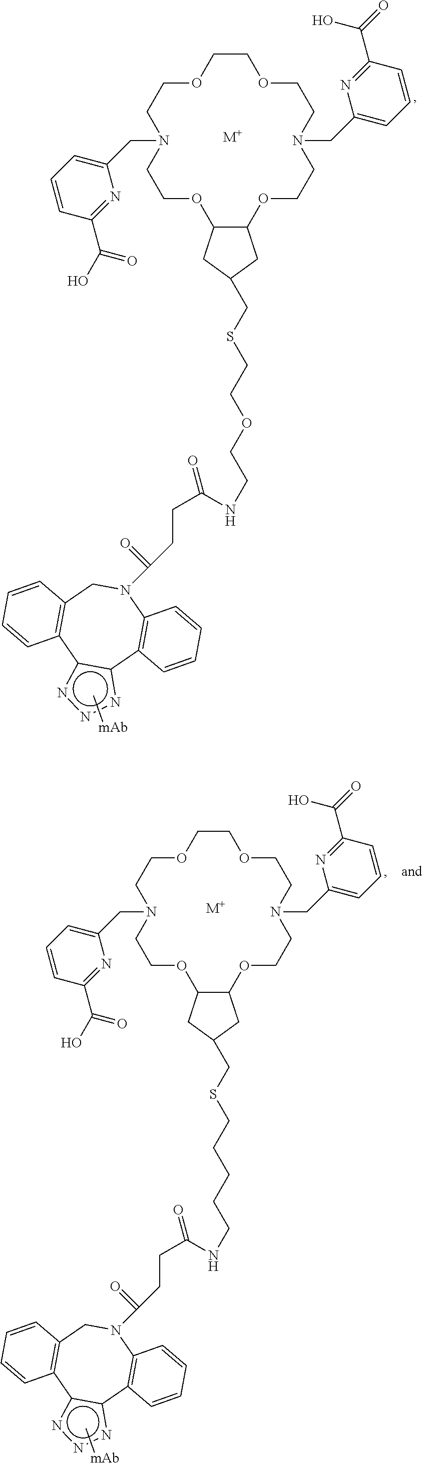

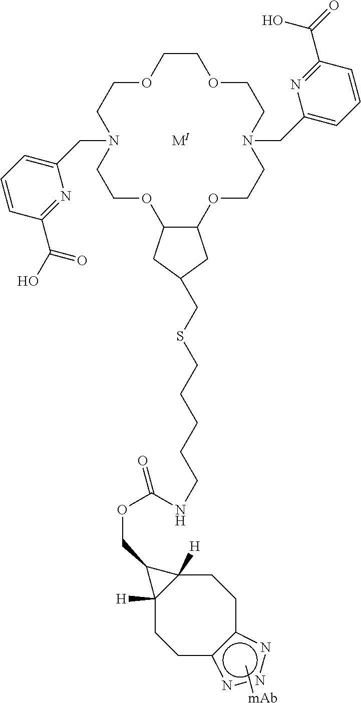

“mAb” preferably refers to an antibody or antigen binding domain that has binding specificity for hK2, such as the Fab of KL2B30, or otherwise described herein.

Radioimmunoconjugates produced by the methods described herein can be analyzed using methods known to those skilled in the art in view of the present disclosure. For example, LC/MS analysis can be used to determine the ratio of the chelator to the labeled polypeptide, e.g., antibody or antigen binding fragment thereof; analytical size-exclusion chromatography can be used to determine the oligomeric state of the polypeptides and polypeptide conjugates, e.g., antibody and antibody conjugates; radiochemical yield can be determined by instant thin layer chromatography (e.g., iTLC-SG), and radiochemical purity can be determined by size-exclusion HPLC.

### Pharmaceutical Compositions and Methods of Use

In another general aspect, the invention relates to a pharmaceutical composition comprising a chelator, radiometal complex, an immunoconjugate, or radioimmunoconjugate of the invention, and a pharmaceutically acceptable carrier. The pharmaceutical composition may comprise one or more pharmaceutically acceptable excipients.

In one embodiment, a pharmaceutical composition comprises a radiometal complex of the invention, and a pharmaceutically acceptable carrier.

In another embodiment, a pharmaceutical composition comprises a radioimmunoconjugate of the invention, and a pharmaceutically acceptable carrier.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition according to the invention. According to particular embodiments, in view of the present disclosure, any pharmaceutically acceptable carrier suitable for use in an antibody-based, or a radiocomplex-based pharmaceutical composition can be used in the invention.

According to particular embodiments, the compositions described herein are formulated to be suitable for the intended route of administration to a subject. For example, the compositions described herein can be formulated to be suitable for parenteral administration, e.g., intravenous, subcutaneous, intramuscular or intratumoral administration.

In other general aspects, the invention relates to methods of selectively targeting neoplastic cells for radiotherapy and treating neoplastic diseases or disorders. Any of the radiocomplexes or radioimmunoconjugates, and pharmaceutical compositions thereof described herein can be used in the methods of the invention.

A “neoplasm” is an abnormal mass of tissue that results when cells divide more than they should or do not die when they should. Neoplasms can be benign (not cancer) or malignant (cancer). A neoplasm is also referred to as a tumor. A neoplastic disease or disorder is a disease or disorder associated with a neoplasm, such as cancer. Examples of neoplastic disease or disorders include, but are not limited to, disseminated cancers and solid tumor cancers.

According to an embodiment, a method of treating prostate cancer (e.g., metastatic prostate cancer, or metastatic castration-resistant prostate cancer) in a subject in need thereof comprises administering to the subject a therapeutically effective amount of a radioimmunoconjugate as described herein, wherein the radioimmunoconjugate comprises a radiometal complex as described herein conjugated to an antigen binding domain with binding specificity for hK2, such as the KL2B30 Fab.

In an embodiment of the invention, a method of selectively targeting neoplastic cells for radiotherapy comprises administering to a subject in need thereof a radioimmunoconjugate or pharmaceutical composition of the invention to the subject.

In an embodiment of the invention, a method of treating a neoplastic disease or disorder comprises administering to a subject in need thereof a radioimmunoconjugate or pharmaceutical composition of the invention to the subject.

In an embodiment of the invention, a method of treating cancer in a subject in need thereof comprises administering to the subject in need thereof a radioimmunoconjugate or pharmaceutical composition of the invention to the subject.

Radioimmunoconjugates carry radiation directly to, for example, cells, etc., targeted by the antigen binding domain. Preferably, the radioimmunoconjugates carry alpha-emitting radiometal ions, such as ²²⁵Ac. Upon targeting, alpha particles from the alpha-emitting radiometal ions, e.g., ²²⁵Ac and daughters thereof, are delivered to the targeted cells and cause a cytotoxic effect thereto, thereby selectively targeting neoplastic cells for radiotherapy and/or treating the neoplastic disease or disorder.

Pre-targeting approaches for selectively targeting neoplastic cells for radiotherapy and for treating a neoplastic disease or disorder are also contemplated by the invention. According to a pre-targeting approach, an azide-labeled antibody or antigen binding fragment thereof is dosed, binds to cells bearing the target antigen of the antibody, and is allowed to clear from circulation over time or removed with a clearing agent. Subsequently, a radiocomplex of the invention, preferably a radiocomplex comprising a cyclooctyne or cyclooctyne derivative, e.g., DBCO, is administered and undergoes a SPAAC reaction with azide-labeled antibody bound at the target site, while the remaining unbound radiocomplex clears rapidly from circulation. The pre-targeting technique provides a method of enhancing radiometal ion localization at a target site in a subject.

In other embodiments, a modified polypeptide, e.g., azide-labeled antibody or antigen binding fragment thereof, and a radiocomplex of the invention are administered to a subject in need of targeted radiotherapy or treatment of a neoplastic disease or disorder in the same composition, or in different compositions.

In some embodiments, a therapeutically effective amount of a radioimmunoconjugate or pharmaceutical composition of the invention is administered to a subject to treat a neoplastic disease or disorder in the subject, such as cancer.

In other embodiments of the invention, radioimmunoconjugates and pharmaceutical compositions of the invention can be used in combination with other agents that are effective for treatment of neoplastic diseases or disorders.

Also provided are radioimmunoconjugates and pharmaceutical compositions as described herein for use in selectively targeting neoplastic cells for radiotherapy and/or for treating a neoplastic disease or disorder and/or for diagnosing a neoplastic disease or disorder; and use of a radioimmunoconjugate or pharmaceutical compositions as described herein in the manufacture of a medicament for selectively targeting neoplastic cells for radiotherapy and/or for treating a neoplastic disease or disorder.

### EXAMPLES

The following examples of the invention are to further illustrate the nature of the invention. It should be understood that the following examples do not limit the invention and that the scope of the invention is to be determined by the appended claims.

### Synthesis of Chelators and Immunoconjugates (Examples 1-21)

Methods of synthesis for embodiments of chelators described herein are provided, for example, in WO2020/229974, which is incorporated by reference herein. Additional synthetic methods are provided in PCT/IB2021/060350, which is incorporated by reference herein, and below in Examples 1-21.

In Examples 1-21, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like.
Abbreviations used in the specification, particularly the Schemes and Examples, are as listed in the Table A, below:

TABLE A
Abbreviations

| | |
|---|---|
| ADP= | Adenosine Diphosphate |
| Alexa633 tracer= | Alexa Fluor ® 633 Hydrazide Tracer (ThermoFisher) |
| BSA= | Bovine Serum Albumin |
| ACN or MeCN= | Acetonitrile |
| ATP= | Adenosine Triphosphate |
| BINAP= | (2,2′-Bis(diphenylphosphino)-1,1′-binaphthyl) |
| Brij ™-35= | Polyethylene glycol hexadecyl ether |
| DBCO= | Dibenzocyclooctyl |
| DCM= | Dichloromethane |
| DIPEA or DIEA= | Diisopropylethylamine |
| DMF= | N,N-Dimethylformamide |
| DMSO= | Dimethylsulfoxide |
| DPPF or dppf= | 1,1′-Bis(diphenylphosphino)ferrocene |
| DTPA= | Diethylene triamine pentaacetic acid |
| DTT= | Dithiothrietol |
| EDTA= | Ethylenediaminetetracetic acid |
| eGFR= | Estimated Glomular Filtration Rate |
| EtOH= | Ethanol |
| F12 medium= | Gibco ® F12 Nutrient Medium (ThermoFisher) |
| FBS= | Fetal Bovine Serum |
| G418= | Geneticin ® (G418) Sulfate |
| GFR= | Glomular Filtration Rate |
| GLP-1= | Glucagon-like peptide 1 |
| GRK2= | G protein-coupled Receptor Kinase 2 |
| HATU= | (1-[Bis(dimethylamino)methylene-1H-1,2,3- |
| | triazolo[4,5-b]pyridinium 3-oxid hexafluorophosphate |
| HBSS= | GIBCO ® Hank's Balanced Salt Solution |
| HEPES= | 4-(2-Hydroxyethyl)-1-Piperizine Ethane Sulfonic Acid |
| HPLC= | High Pressure Liquid Chromatography |
| HTRF= | Homogeneous Time Resolved Fluorescence |
| IFG= | Impaired fasting glucose |
| IGT= | Impaired glucose tolerance |
| LCMS or LC/MS= | Liquid chromatography-mass spectrometry |
| LDA= | Lithium diisopropylamide |
| LiHMDS= | Lithium bis(trimethylsilyl)amide |
| MeOH= | Methanol |
| mesyl or Ms= | Methylsulfonyl (i.e. —SO₂—CH₃) |
| mesylate or OMs= | Methanesulfonate (i.e. —O—SO₂—CH₃) |
| MOM= | Methoxy methyl |
| Ms or mesyl= | —SO₂—CH₃ |
| MsCl= | Mesyl Chloride (i.e. CH₃—SO₂—Cl) |
| NaBH(OAc)₃= | Sodium triacetoxyborohydride |
| NAFLD= | Non-alcoholic fatty liver disease |
| Na₂SO₄= | Sodium Sulfate |
| NASH= | Non-alcoholic steatohepatitis |
| NBS= | N-Bromosuccinimide |
| NH(PMB)₃= | tris(4-methoxybenzyl)-λ⁴-azane |
| NMO= | 4-Methylmorpholine N-oxide |
| NMP= | N-Methyl-2-pyrrolidone |
| NMR= | Nuclear Magnetic Resonance |
| OMs or mesylate= | Methanesulfonate (i.e. —O—SO₂—CH₃) |
| OTf or triflate= | Trifluoromethanesulfonate |
| OTs or tosylate= | p-Toluenesulfonate |
| Pd/C= | Palladium on carbon |
| Pd(dba)₂= | Bis(dibenzylideneacetone)dipalladium(0) |
| Pd(dppf)Cl₂= | [1,1′-Bis(diphenylphosphino)ferrocene] Palladium |
| | (II) Dichloride |
| Pd(dppf)Cl₂•CHCl₃= | [1,1′-Bis(diphenylphosphino)ferrocene]chloropalladium |
| | complex with chloroform |
| (1-nap)₃P or | Tri(1-naphthyl)phosphine |
| P(1-nap)₃= | |
| Pd(OAc)₂= | Palladium (II) acetate |
| Pd(OH)₂= | Palladium hydroxide |
| Pd(OH)₂/C= | Palladium hydroxide on carbon (Pearlman's Catalyst) |
| Pd(PPh₃)₄= | Tetrakis(triphenylphosphine) palladium (0) |
| PEG= | Polyethylene Glycol |
| PMB= | 4-Methoxybenzyl ether |
| PPh₃= | Triphenylphosphine |
| SNS= | Sympathetic Nervous System |
| TBAB= | Tetra-n-butylammonium bromide |
| TBAF= | Tetra-n-butylammonium fluoride |
| TBAI= | Tetra-n-butylammonium iodide |
| TBSOTf= | Tert-butyldimethylsilyl trifluoromethanesulfonate |
| TEA= | Triethylamine |
| Tf or trifyl= | Trifluoromethylsulfonyl (i.e. —SO₂—CF₃) |
| TFA= | Trifluoroacetic Acid |
| THF= | Tetrahydrofuran |
| THP= | 2-Tetrahydropyranyl |
| TLC= | Thin Layer Chromatography |
| TMS= | Trimethylsilyl |
| TMSN₃= | Trimethylsilyl azide |
| Tosylate or OTs= | p-Toluenesulfonate |
| TOPA= | 6,6′-((1,4,10,13-tetraoxa-7,16-diazacyclooctadecane- |
| | 7,16-diyl)bis(methylene))dipicolinic acid = H2bp18c6 |
| Ts or tosyl= | -p-Toluenesulfonyl chloride |
| p-TsCl= | p-Toluenesulfonyl chloride |
| Tween-20 ®= | Nonionic detergent (Sigma Aldrich) |

As used herein, unless otherwise noted, the term “isolated form” shall mean that the compound is present in a form which is separate from any solid mixture with another compound(s), solvent system or biological environment. In an embodiment of the present invention, any of the compounds as herein described are present in an isolated form.

As used herein, unless otherwise noted, the term “substantially pure form” shall mean that the mole percent of impurities in the isolated compound is less than about 5 mole percent, preferably less than about 2 mole percent, more preferably, less than about 0.5 mole percent, most preferably, less than about 0.1 mole percent. In an embodiment of the present invention, the compound of formula (I) is present as a substantially pure form.

As used herein, unless otherwise noted, the term “substantially free of a corresponding salt form(s)” when used to described the compound of formula (I) shall mean that mole percent of the corresponding salt form(s) in the isolated base of formula (I) is less than about 5 mole percent, preferably less than about 2 mole percent, more preferably, less than about 0.5 mole percent, most preferably less than about 0.1 mole percent. In an embodiment of the present invention, the compound of formula (I) is present in a form which is substantially free of corresponding salt form(s).

### Example 1

### 4-((6-(methoxycarbonyl)pyridin-2-yl)(16-((6-(methoxycarbonyl)pyridin-2-yl)methyl)-1,4,10,13-tetraoxa-7,16-diazacyclooctadecan-7-yl)methyl)benzoic acid

### TOPA-[C-7]-Phenyl-carboxylic acid

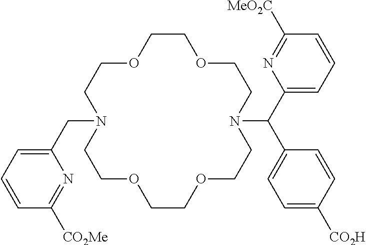 [SMILES: COC(=O)c1cccc(CN2CCOCCOCCN(C(c3ccc(C(=O)O)cc3)c3cccc(C(=O)OC)n3)CCOCCOCC2)n1]

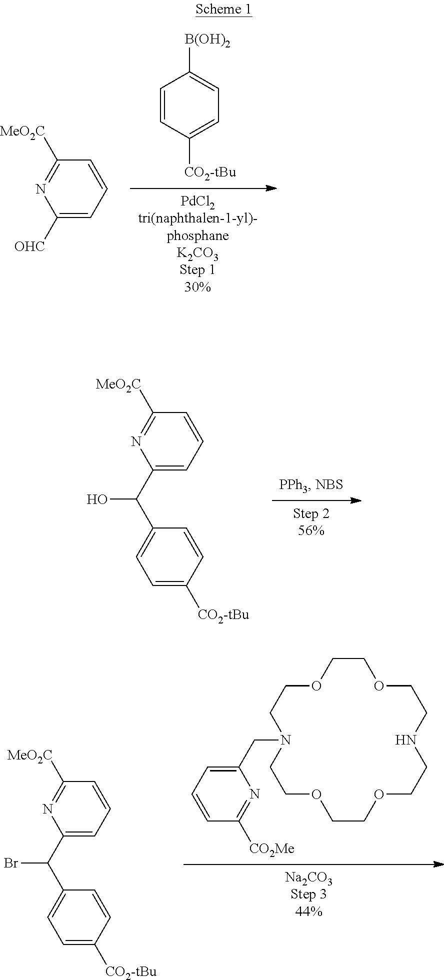

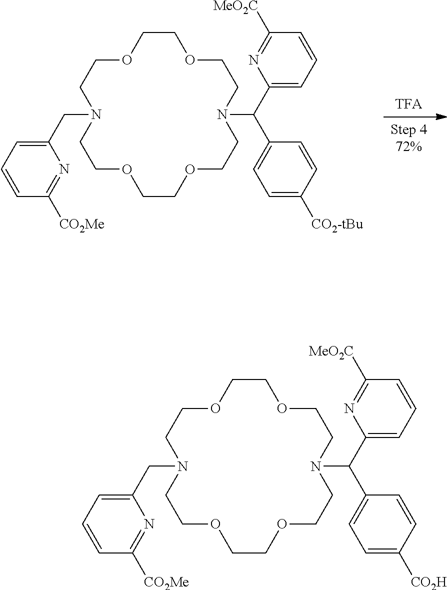

Step 1: To a mixture of methyl 6-formylpicolinate (4.00 g, 24.2 mmol), (4-(tert-butoxycarbonyl)phenyl)boronic acid (10.7 g, 48.5 mmol), PdCl₂ (0.21 g, 1.2 mmol), tri(naphthalen-1-yl)phosphine (0.50 g, 1.2 mmol) and potassium carbonate (10.0 g, 72.7 mmol) under nitrogen at −78° C. in a 500 mL three neck round bottom flask was added tetrahydrofuran (100 mL) in one portion. The mixture was purged with nitrogen and stirred at room temperature for 30 min, then heated at 65° C. for 24 h. The reaction mixture was cooled room temperature and filtered through a pad of Celite and the filtrate was concentrated to dryness. The crude product was purified by silica gel chromatography (0-50% EtOAc/petroleum ether) to afford methyl 6-((4-(tert-butoxycarbonyl)phenyl)(hydroxy)methyl)picolinate as a yellow oil (2.5 g, 30% yield).

Step 2: A stir bar, methyl 6-((4-(tert-butoxycarbonyl)phenyl)(hydroxy)methyl)picolinate (2.50 g, 7.30 mmol), PPh₃ (3.43 g, 13.1 mmol), N-bromosuccinimide (2.13 g, 12.0 mmol) and dichloromethane (30 mL) were added to a 250 mL three neck round bottom flask under nitrogen atmosphere at room temperature and stirred for 1 h. The reaction solution was loaded onto a silica gel column and chromatography (0-30% EtOAc/petroleum ether) gave compound methyl 6-(bromo(4-(tert-butoxycarbonyl)phenyl)methyl)picolinate (1.65 g, 56% yield) as a yellow oil.

Step 3: A stir bar, methyl 6-(bromo(4-(tert-butoxycarbonyl)phenyl)methyl)picolinate (1.52 g, 3.69 mmol), methyl 6-((1,4,10,13-tetraoxa-7,16-diazacyclooctadecan-7-yl)methyl)picolinate (1.50 g, 3.69 mmol), Na₂CO₃ (1.17 g, 11.1 mmol), and acetonitrile (30 mL) were added to a 250 mL three neck round-bottomed flask, and the resultant heterogeneous mixture was heated at 90° C. for 16 h under nitrogen atmosphere. Subsequently reaction mixture was cooled to room temperature, filtered through a pad of Celite, and concentrated to dryness in vacuo to give the crude product. The crude product was purified by silica gel chromatography (0-10% MeOH/dichloromethane) to afford methyl 6-((4-(tert-butoxycarbonyl)phenyl)(16-((6-(methoxycarbonyl)pyridin-2-yl)methyl)-1,4,10,13-tetraoxa-7,16-diazacyclooctadecan-7-yl)methyl)picolinate as a brown oil (1.2 g, 44%).

Step 4: A stir bar, methyl 6-((4-(tert-butoxycarbonyl)phenyl)(16-((6-(methoxycarbonyl)pyridin-2-yl)methyl)-1,4,10,13-tetraoxa-7,16-diazacyclooctadecan-7-yl)methyl)picolinate (1.2 g, 1.6 mmol), TFA (0.62 mL, 8.1 mmol) and DCM (20 mL) were added to a 100 mL three neck round bottom flask at r.t. and stirred for 1 h. Reaction mixture was concentrated to dryness and the resultant crude product was subjected to preparative HPLC (Column: XBRIDGE C18 (19×150 mm) 5.0 μm; Mobile phase: 0.1% TFA in water/ACN; Flow Rate: 15.0 mL/min) to give TOPA-[C-7]-Phenyl-carboxylic acid (0.8 g, 72%) as brown oil. LC-MS APCI: Calculated for C₃₅H₄₄N₄O₁₀ 680.31; Observed m/z [M+H]⁺ 681.5. Purity by LC-MS: 99.87%. Purity by HPLC: 97.14% (97.01% at 210 nm, 97.20% at 254 nm and 97.21% at 280 nm; Column: Atlantis dC18 (250×4.6 mm), 5 μm; Mobile phase A: 0.1% TFA in water, Mobile phase B: acetonitrile; Flow rate: 1.0 mL/min.%. ¹H NMR (400 MHz, DMSO-d6): δ 8.12-8.07 (m, 4H), 8.00-7.98 (m, 2H), 7.75-7.73 (m, 4H), 6.10 (s, 1H), 4.67 (s, 2H), 3.96 (s, 3H), 3.91 (s, 3H), 3.82 (s, 8H), 3.56 (s, 8H), 3.52 (s, 8H).

### Example 2

### 6-((16-((6-Carboxypyridin-2-yl)(4-((2-(2-(2-isothiocyanatoethoxy)ethoxy)ethyl)carbamoyl)phenyl)methyl)-1,4,10,13-tetraoxa-7,16-diazacyclooctadecan-7-yl)methyl)picolinic acid

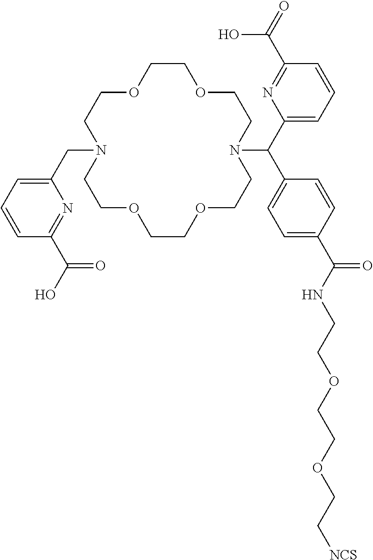

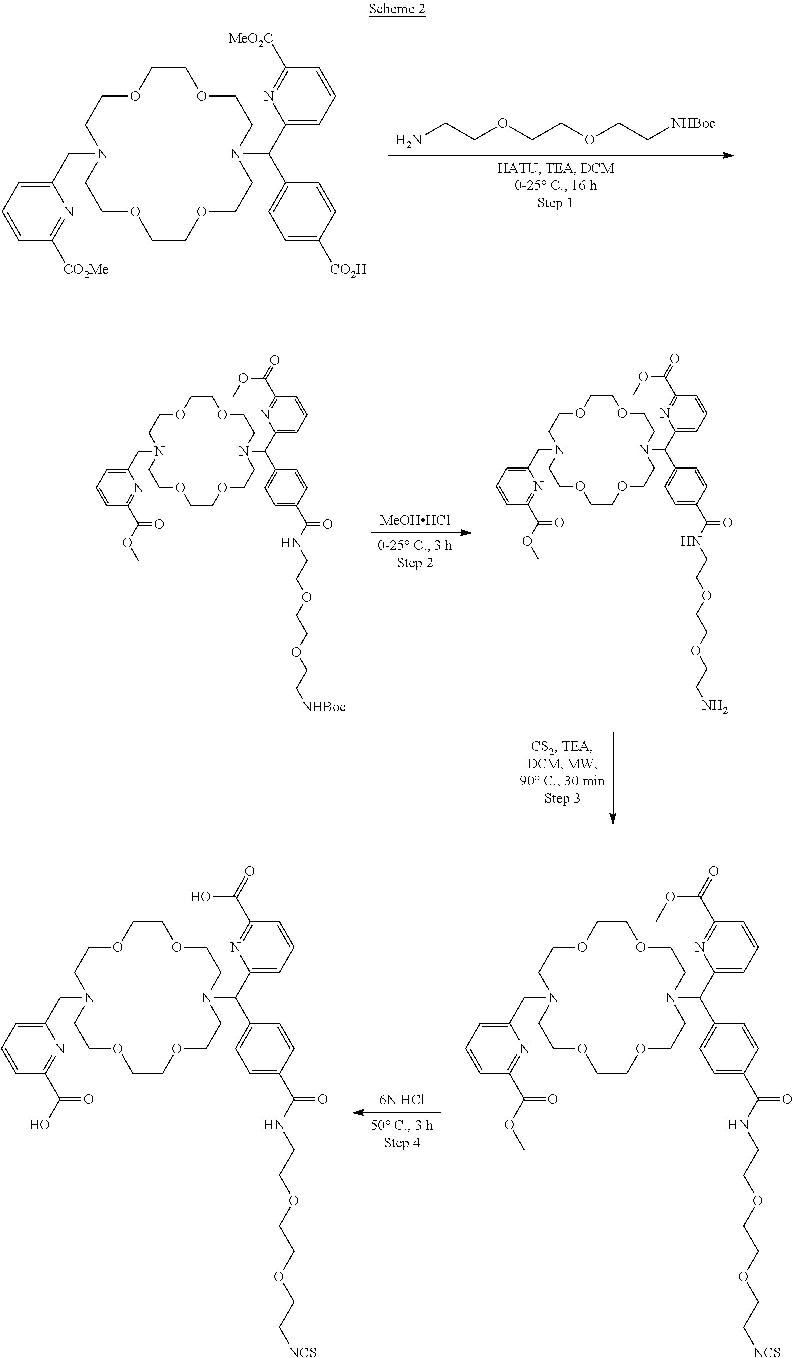

Step 1: A stir bar, 4-((6-(methoxycarbonyl)pyridin-2-yl)(16-((6-(methoxycarbonyl)pyridin-2-yl)methyl)-1,4,10,13-tetraoxa-7,16-diazacyclooctadecan-7-yl)methyl)benzoic acid (0.40 g, 0.60 mmol), tert-butyl (2-(2-(2-aminoethoxy)ethoxy)ethyl)carbamate (0.15 g, 0.60 mmol), triethylamine (0.18 g, 0.76 mmol), HATU (0.33 g, 0.90 mmol), and DCM (4.0 mL) were added to a 25 mL three neck round-bottomed flask at 0° C. under nitrogen atmosphere. The mixture was stirred overnight at room temperature. The reaction was treated with water (10 mL) and extracted with dichloromethane (10 mL×3). The combined extracts were washed with 10% aqueous NaHCO₃ (10 mL), brine (10 mL), dried over anhydrous Na₂SO₄, filtered, and concentrated to dryness to yield an oil, which was purified by silica gel chromatography (0-10% MeOH/DCM) to yield methyl 6-((4-((2,2-dimethyl-4-oxo-3,8,11-trioxa-5-azatridecan-13-yl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.18 g).

Step 2: A stir bar, methyl 6-((4-((2,2-dimethyl-4-oxo-3,8,11-trioxa-5-azatridecan-13-yl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.18 g, 0.20 mmol), MeOH (1.8 mL), and HCl in methanol (4 M, 1.0 mL, 4.0 mmol) were added to a 10 mL single-neck round-bottomed flask at 0° C., then warmed to room temperature and stirred for 2 h. The volatiles were removed in vacuo to yield methyl 6-((4-((2-(2-(2-aminoethoxy)ethoxy)ethyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.15 g), which was used without purification.

Step 3: A stir bar, methyl 6-((4-((2-(2-(2-aminoethoxy)ethoxy)ethyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.10 g, 0.12 mmol), triethylamine (37 mg, 0.37 mmol), dry DCM (2 mL), and carbon disulfide (14 mg, 0.18 mmol) were added to a pressure vial at room temperature under a nitrogen atmosphere. The vial was subjected to microwave-irradiation (150 W power) at 90° C. for 30 min. The vial was then cooled to room temperature, the reaction mixture diluted with dichloromethane (10 mL), and then washed successively with water (5 mL), 1 M HCl (5 mL), and water (5 mL), dried over anhydrous Na₂SO₄, filtered, and concentrated to dryness to yield methyl 6-((4-((2-(2-(2-isothiocyanatoethoxy)ethoxy)ethyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (100 mg), which was used without purification.

Step 4: A stir bar, methyl 6-((4-((2-(2-(2-isothiocyanatoethoxy)ethoxy)ethyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.10 g, 0.12 mmol), and aqueous HCl (6 N, 0.4 mL, 2.34 mmol) were added to a 10 mL single-neck round-bottomed flask, and stirred at 50° C. for 3 h. The reaction mixture was cooled to room temperature, concentrated to dryness in vacuo to yield an oil, which was purified by preparative HPLC (Column: XBRIDGE C18 19×150 mm, 5.0 μm; Mobile phase: 0.1% TFA in water/acetonitrile; Flow Rate: 15.0 mL/min) to yield 6-((16-((6-carboxypyridin-2-yl)(4-((2-(2-(2-isothiocyanatoethoxy)ethoxy)ethyl)carbamoyl)phenyl)methyl)-1,4,10,13-tetraoxa-7,16-diazacyclooctadecan-7-yl)methyl)picolinic acid (5.0 mg). LC-MS APCI: Calculated for C₄₀H₅₂N₆O₁₁S: 824.34; Observed m/z [M+H]⁺ 824.8. ¹H NMR (400 MHz, CD₃OD): δ 8.22-8.20 (m, 2H), 8.14-8.05 (m, 2H), 7.94 (d, J=8.00 Hz, 2H), 7.79 (d, J=8.00 Hz, 2H), 7.73-7.67 (m, 2H), 6.16 (s, 1H), 4.77 (s, 2H), 3.93-4.00 (m, 8H), 3.59-3.70 (m, 27H), 3.47-3.44 (m, 2H).

### Example 3

### 6-((4-((6-Aminohexyl)carbamoyl)phenyl)(16-((6-carboxypyridin-2-yl)methyl)-1,4,10,13-tetraoxa-7,16-diazacyclooctadecan-7-yl)methyl)picolinic acid and

### Example 4

### 6-((16-((6-Carboxypyridin-2-yl)(4-((6-isothiocyanatohexyl)carbamoyl)phenyl)methyl)-1,4,10,13-tetraoxa-7,16-diazacyclooctadecan-7-yl)methyl)picolinic acid

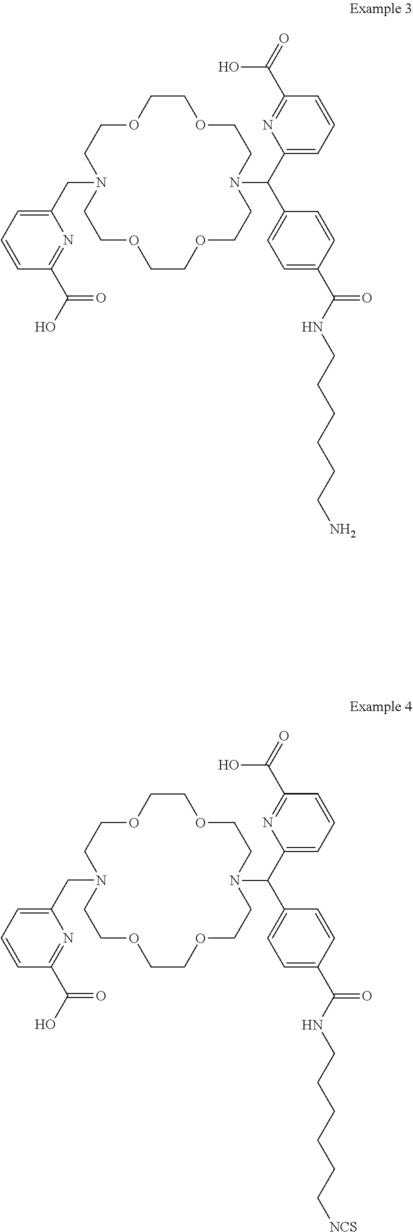

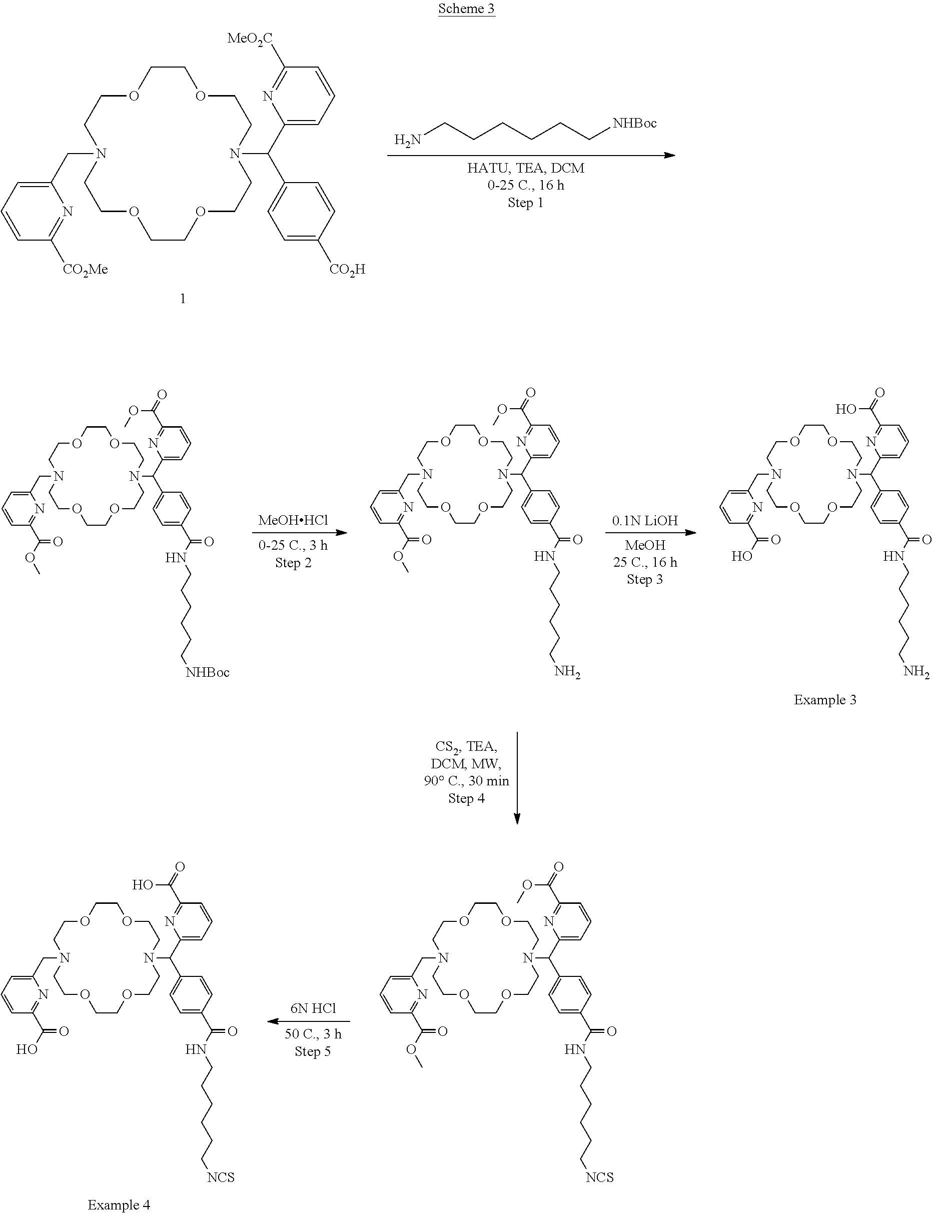

Step 1: A stir bar, 4-((6-(methoxycarbonyl)pyridin-2-yl)(16-((6-(methoxycarbonyl)pyridin-2-yl)methyl)-1,4,10,13-tetraoxa-7,16-diazacyclooctadecan-7-yl)methyl)benzoic acid (0.12 g, 0.18 mmol), tert-butyl (6-aminohexyl)carbamate (38 mg, 0.18 mmol), triethylamine (54 mg, 0.54 mmol), HATU (0.10 g, 0.27 mmol), and DCM (4.0 mL) were added to a 25 mL three-neck round-bottomed flask at 0° C. under a nitrogen atmosphere. The reaction mixture was then brought to room temperature and stirred overnight. The reaction mixture was then treated with water (10 mL) and extracted with dichloromethane (10 mL×3). The combined extracts were washed with 10% aqueous NaHCO₃ (10 mL) and brine (10 mL), dried over anhydrous Na₂SO₄, filtered, and concentrated to dryness to yield an oil. The oil was purified via silica gel chromatography (0-10% MeOH/DCM) to yield methyl 6-((4-((6-((tert-butoxycarbonyl)amino)hexyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (70 mg) as a gummy oil.

Step 2: A stir bar, methyl 6-((4-((6-((tert-butoxycarbonyl)amino)hexyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (70 mg, 0.080 mmol), MeOH (1.5 mL), and HCl in methanol (4 M, 0.4 mL, 1.6 mmol) were added to a 25 mL round-bottomed flask at 0° C., which was subsequently brought to room temperature and stirred for 2 h. The volatiles were removed in vacuo to yield methyl 6-((4-((6-aminohexyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (30 mg), which was used without purification.

Step 3: A stir bar, methyl 6-((4-((6-aminohexyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (30 mg, 0.038 mmol), aqueous LiOH (1.1 mL, 0.1 N, 0.11 mmol), and MeOH (1.0 mL) were added to an 8 mL reaction vial and stirred overnight at room temperature. The reaction mixture was then treated with acetic acid until pH-6.5, and subsequently concentrated to dryness in vacuo at room temperature. The resultant product was subjected to preparative HPLC (Column: XBRIDGE C18 19×150 mm, 5.0 μm; Mobile phase: 10 mM ammonium acetate in water/ACN; Flow Rate: 15.0 mL/min) to yield Example 3: 6-((4-((6-aminohexyl)carbamoyl)phenyl)(16-((6-carboxypyridin-2-yl)methyl)-1,4,10,13-tetraoxa-7,16-diazacyclooctadecan-7-yl)methyl)picolinic acid (10 mg). LC-MS APCI: Calculated for C₃₉H₅₄N₆O₉; 750.40; Observed m/z [M+H]⁺ 751.3. ¹H NMR (400 MHz, CD₃OD): δ 8.22 (d, J=1.60 Hz, 2H), 8.21-8.06 (m, 2H), 7.92 (d, J=8.40 Hz, 2H), 7.80 (d, J=8.40 Hz, 2H), 7.75-7.69 (m, 2H), 6.20 (s, 1H), 4.70 (s, 2H), 4.02-3.92 (m, 8H), 3.76-3.62 (m, 14H), 3.51-3.32 (m, 4H), 2.93 (t, J=8.00 Hz, 2H), 1.67-1.64 (m, 4H), 1.46-1.45 (m, 4H).

Step 4: A stir bar, methyl 6-((4-((6-aminohexyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.10 g, 0.13 mmol), triethylamine (39 mg, 0.38 mmol), dry DCM (2 mL), and carbon disulfide (15 mg, 0.19 mmol) were added to a pressure vial at room temperature under a nitrogen atmosphere. The vial was subjected to microwave irradiation (150 W power) at 90° C. for 30 min. The vial was then cooled to room temperature and the reaction mixture diluted with dichloromethane (10 mL), washed with water (5 mL), 1 M HCl (5 mL), and water (5 mL), dried over anhydrous Na₂SO₄, filtered, and concentrated to dryness to yield methyl 6-((4-((6-isothiocyanatohexyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.1 g), which was used without purification.

Step 5: A stir bar, methyl 6-((4-((6-isothiocyanatohexyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.10 g, 0.12 mmol), and aqueous HCl (6 N, 0.4 mL, 2.4 mmol) were added to a 10 mL round-bottomed flask, and then stirred at 50° C. for 3 h. The reaction mixture was then cooled to room temperature and concentrated to dryness in vacuo to yield a residue, which was purified by preparative HPLC (Column: XBRIDGE C18 19×150 mm, 3.5 μm; Mobile phase: 0.1% TFA in water/acetonitrile; Flow Rate: 2.0 mL/min) to yield Example 4: 6-((16-((6-carboxypyridin-2-yl)(4-((6-isothiocyanatohexyl)carbamoyl)phenyl)methyl)-1,4,10,13-tetraoxa-7,16-diazacyclooctadecan-7-yl)methyl)picolinic acid (15 mg). LC-MS APCI: Calculated for C₄₀H₅₂N₆O₉S: 792.35; Observed m/z [M+H]⁺ 792.8. ¹H NMR (400 MHz, CD₃OD): δ 8.23-8.20 (m, 2H), 8.15-8.06 (m, 2H), 7.92 (d, J=8.40 Hz, 2H), 7.79 (d, J=8.40 Hz, 2H), 7.74-7.68 (m, 2H), 6.17 (s, 1H), 4.77 (s, 2H), 4.01-3.93 (m, 8H), 3.75-3.56 (m, 16H), 3.42-3.33 (m, 5H), 1.74-1.64 (m, 4H), 1.50-1.44 (m, 4H).

### Example 5

### 6-((16-((6-Carboxypyridin-2-yl)(4-((4-isothiocyanatophenethyl)carbamoyl)phenyl)methyl)-1,4,10,13-tetraoxa-7,16-diazacyclooctadecan-7-yl)methyl)picolinic acid

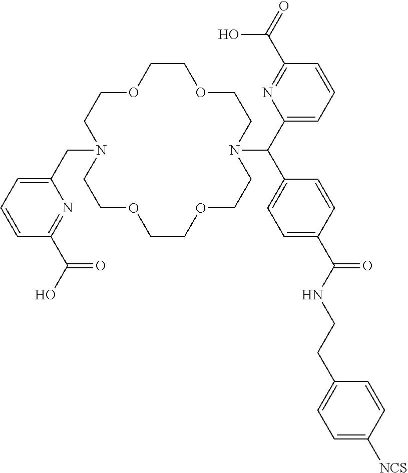

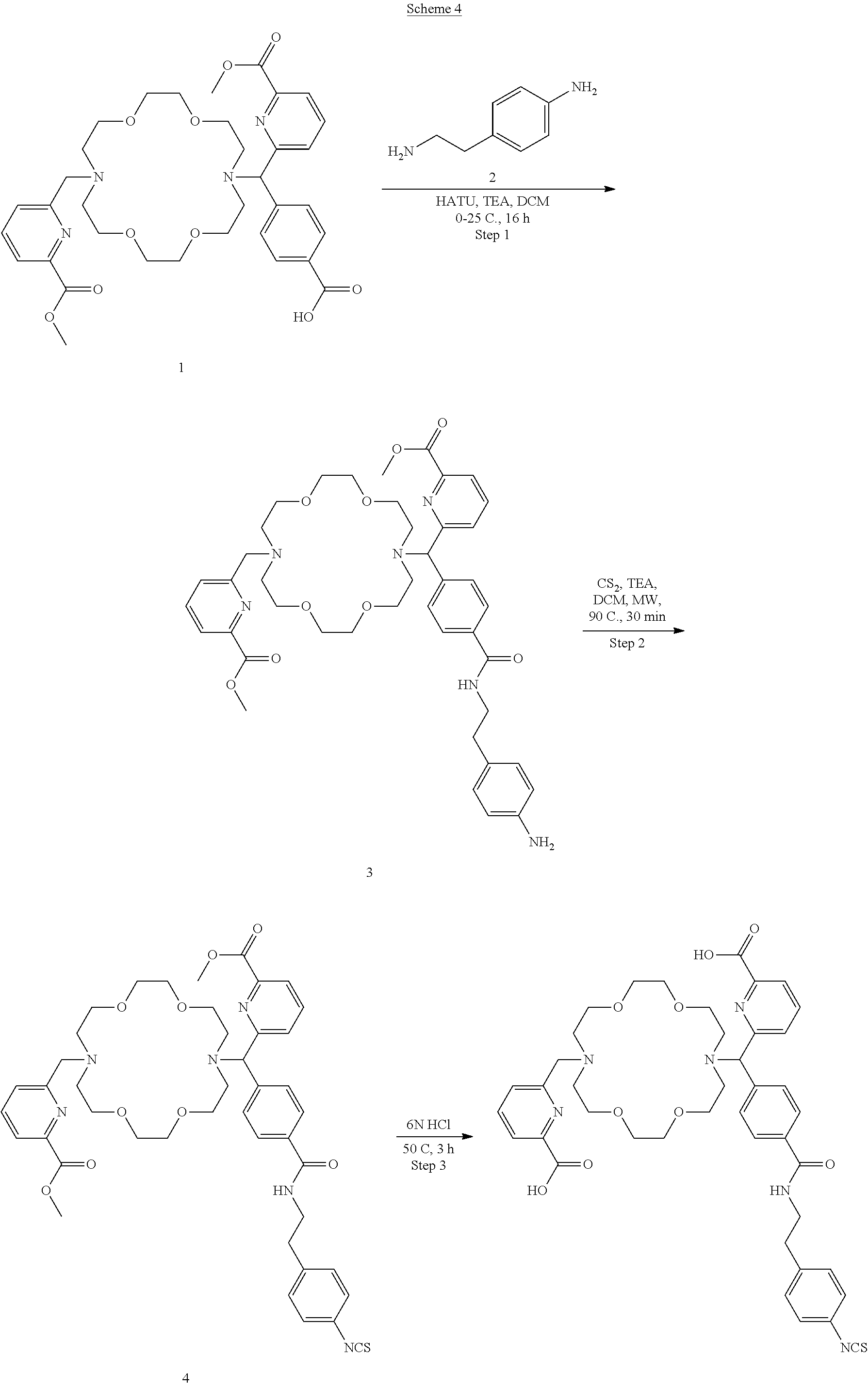

Step 1: A stir bar, 4-((6-(methoxycarbonyl)pyridin-2-yl)(16-((6-(methoxycarbonyl)pyridin-2-yl)methyl)-1,4,10,13-tetraoxa-7,16-diazacyclooctadecan-7-yl)methyl)benzoic acid (0.25 g, 0.37 mmol), 4-(2-aminoethyl)aniline (60 mg, 0.37 mmol), TEA (0.11 g, 0.15 mL, 1.1 mmol), HATU (0.21 g, 0.55 mmol), and DCM (5 mL) were added to a 25 mL three neck round-bottomed flask at 0° C. under a nitrogen atmosphere. The reaction mixture was stirred overnight at room temperature, and then treated with water (10 mL), and extracted with dichloromethane (10 mL×3). The combined extracts were washed with 10% aqueous NaHCO₃ (10 mL) and brine (10 mL), dried over anhydrous Na₂SO₄, filtered, and concentrated to dryness to yield a product which was purified by silica gel chromatography (0-10% MeOH/DCM) to yield methyl 6-((4-((4-aminophenethyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.12 g).

Step 2: A stir bar, methyl 6-((4-((4-aminophenethyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.12 g, 0.15 mmol), TEA (45 mg, 65 μL, 0.45 mmol), DCM (3 mL), and CS₂ (17 mg, 0.23 mmol) were added to a 10 mL microwave pressure vial at room temperature under a nitrogen atmosphere. The reaction mixture was subjected to microwave-irradiation (150 W power) at 90° C. for 30 min. The reaction mixture was then cooled to room temperature, diluted with dichloromethane (10 mL), washed successively with water (5 mL), 1 M HCl (5 mL), and water (5 mL), dried over anhydrous Na₂SO₄, and concentrated to dryness to yield methyl 6-((4-((4-isothiocyanatophenethyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.12 g), which was used without purification.

Step 3: A stir bar, methyl 6-((4-((4-isothiocyanatophenethyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.12 g, 0.14 mmol), and aqueous HCl (0.50 mL, 6 N, 2.8 mmol) were added to a 10 mL single-neck round-bottomed flask and stirred at 50° C. for 3 h. The reaction mixture was cooled to room temperature, concentrated to dryness in vacuo, and the crude product was subjected to preparative HPLC (Column: XBRIDGE C18 19×150 mm, 5.0 μm; Mobile phase: 0.1% TFA in water/acetonitrile; Flow Rate: 15.0 mL/min) to yield 6-((16-((6-carboxypyridin-2-yl)(4-((4-isothiocyanatophenethyl)carbamoyl)phenyl)methyl)-1,4,10,13-tetraoxa-7,16-diazacyclooctadecan-7-yl)methyl)picolinic acid (30 mg). LC-MS APCI: Calculated for C₄₂H₄₅N₅O₁₀S; 812.32; Observed m/z [M+H]⁺ 812.9. ¹H NMR (400 MHz, CD₃OD): δ 8.22 (d, J=0.80 Hz, 2H), 8.06-8.21 (m, 2H), 7.85 (d, J=8.40 Hz, 2H), 7.68-7.78 (m, 4H), 7.31 (d, J=8.40 Hz, 2H), 7.21 (d, J=2.00 Hz, 2H), 6.18 (s, 1H), 4.77 (s, 2H), 3.70-4.00 (m, 7H), 3.60-3.67 (m, 16H), 3.44-3.49 (m, 2H), 2.90-3.10 (m, 3H).

### Example 6

### (S)-6,6′-((2-(((2-Isothiocyanatoethyl)thio)methyl)-1,4,10,13-tetraoxa-7,16-diazacyclooctadecane-7,16-diyl)bis(methylene))dipicolinic acid

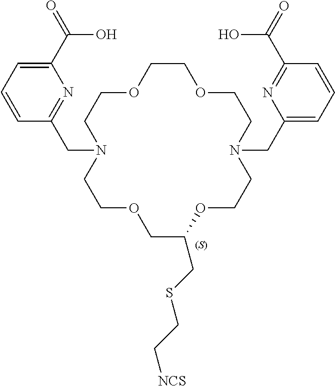

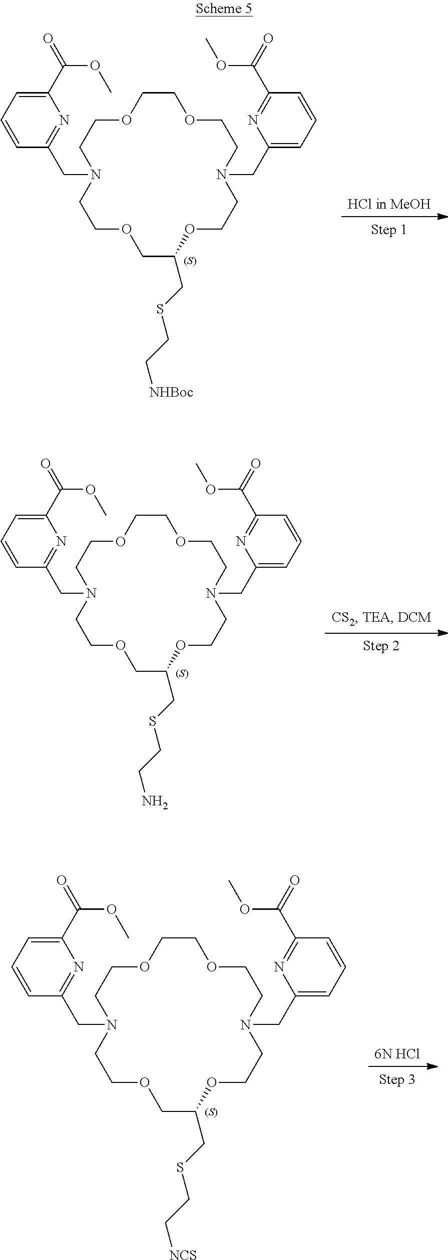

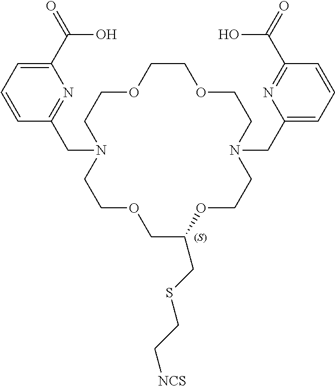

Step 1: A stir bar, 1 (methyl 6-((4-((tert-butoxycarbonyl)amino)phenyl)(16-((6-(methoxycarbonyl)pyridin-2-yl)methyl)-1,4,10,13-tetraoxa-7,16-diazacyclooctadecan-7-yl)methyl)picolinate) (0.10 g, 0.15 mmol), MeOH (0.5 mL) and HCl in methanol (4 M, 0.6 mL, 4.0 mmol) were added to a 25 mL single-neck round-bottomed flask at 0° C. and then brought to room temperature and stirred for 2 h. The volatiles were removed in vacuo to yield dimethyl 6,6′-((2-(((2-aminoethyl)thio)methyl)-1,4,10,13-tetraoxa-7,16-diazacyclooctadecane-7,16-diyl)bis(methylene))(S)-dipicolinate (55 mg), which was used in the next step without purification.

Step 2: A stir bar, dimethyl 6,6′-((2-(((2-aminoethyl)thio)methyl)-1,4,10,13-tetraoxa-7,16-diazacyclooctadecane-7,16-diyl)bis(methylene))(S)-dipicolinate (50 mg, 0.10 mmol), triethylamine (24 mg, 0.24 mmol), DCM (2 mL) and carbon disulfide (12 mg, 0.16 mmol) were added to a microwave vial at room temperature under a nitrogen atmosphere. The vial was subjected to microwave-irradiation (150 W power) at 90° C. for 30 min. The vial was then cooled to room temperature and the reaction mixture diluted with dichloromethane (10 mL), washed successively with water (5 mL), 1M HCl (5 mL), water (5 mL), dried over anhydrous Na₂SO₄, concentrated to dryness and was subjected to silica gel chromatography (0-10% MeOH/DCM) to yield dimethyl 6,6′-((2-(((2-isothiocyanatoethyl)thio)methyl)-1,4,10,13-tetraoxa-7,16-diazacyclooctadecane-7,16-diyl)bis(methylene))(S)-dipicolinate as a yellow solid (20 mg).

Step 3: A stir bar, dimethyl 6,6′-((2-(((2-isothiocyanatoethyl)thio)methyl)-1,4,10,13-tetraoxa-7,16-diazacyclooctadecane-7,16-diyl)bis(methylene))(S)-dipicolinate (20 mg, 0.030 mmol) and aqueous HCl (6 N, 0.1 mL, 0.6 mmol) were added to a 10 mL single-neck round-bottomed flask and stirred at room temperature overnight. The reaction mixture was concentrated to dryness in vacuo, and the resultant residue was subjected to preparative HPLC (Column: XBRIDGE C18 (19×150 mm) 5.0 μm; Mobile phase: 0.1% TFA in water/acetonitrile; Flow Rate: 15.0 mL/min) to yield (S)-6,6′-((2-(((2-isothiocyanatoethyl)thio)methyl)-1,4,10,13-tetraoxa-7,16-diazacyclooctadecane-7,16-diyl)bis(methylene))dipicolinic acid (6 mg). LC-MS APCI: Calculated for C₃₀H₄₁N₅O₈S₂: 663.24; Observed m/z [M+H]⁺ 664.2. ¹H NMR (400 MHz, DMSO-d₆): δ 9.78 (s, 1H), 8.10 (s, 4H), 7.78 (d, J=6.00 Hz, 2H), 4.69 (s, 4H), 3.96-3.52 (m, 23H), 2.85 (t, J=6.40 Hz, 2H), 2.70 (t, J=8.00 Hz, 2H).

### Example 7

### (S)-6,6′-((2-(((5-Isothiocyanatopentyl)thio)methyl)-1,4,10,13-tetraoxa-7,16-diazacyclooctadecane-7,16-diyl)bis(methylene))dipicolinic acid

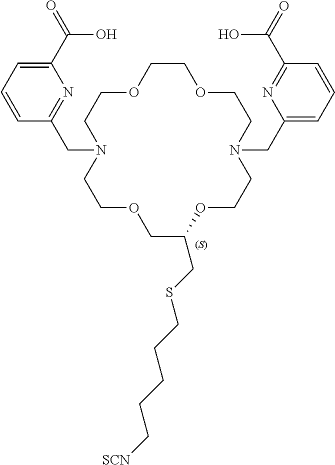

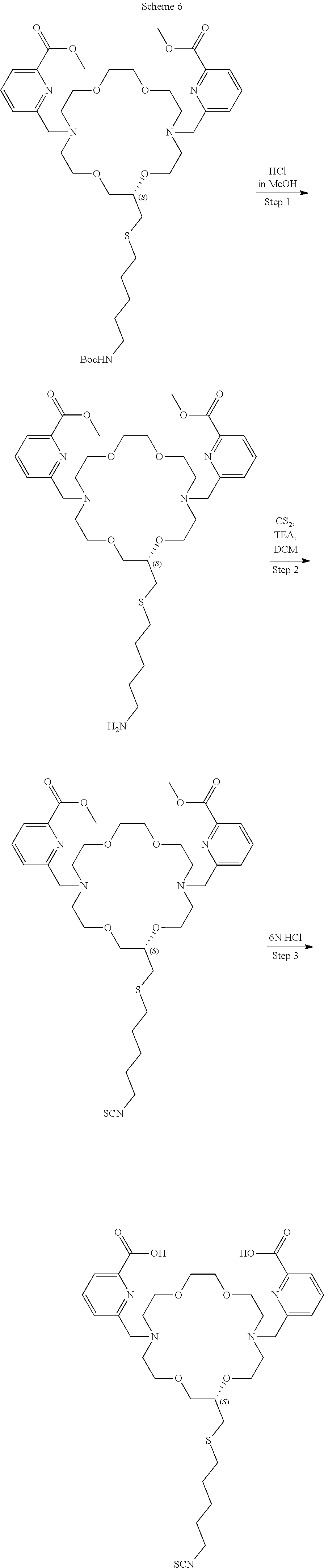

Step 1: A stir bar, dimethyl 6,6′-((2-(((5-((tert-butoxycarbonyl)amino)pentyl)thio)methyl)-1,4,10,13-tetraoxa-7,16-diazacyclooctadecane-7,16-diyl)bis(methylene))(S)-dipicolinate (0.12 g, 0.15 mmol), MeOH (0.5 mL), and HCl in methanol (4 M, 0.6 mL, 4.0 mmol) were added to a 25 mL single-neck round-bottomed flask at 0° C. and brought to room temperature and stirred for 2 h. The volatiles were then removed in vacuo to yield dimethyl 6,6′-((2-(((5-aminopentyl)thio)methyl)-1,4,10,13-tetraoxa-7,16-diazacyclooctadecane-7,16-diyl)bis(methylene))(S)-dipicolinate (70 mg), which was used without purification.

Step 2: A stir bar, dimethyl 6,6′-((2-(((5-aminopentyl)thio)methyl)-1,4,10,13-tetraoxa-7,16-diazacyclooctadecane-7,16-diyl)bis(methylene))(S)-dipicolinate (70 mg, 0.10 mmol), triethylamine (20 mg, 0.20 mmol) dry DCM (2 mL) and carbon disulfide (15 mg, 0.20 mmol) were added to a microwave vial at room temperature under a nitrogen atmosphere. The reaction mixture was subjected to microwave-irradiation (150 W power) at 90° C. for 30 min. The vial was brought to room temperature and the reaction mixture was diluted with dichloromethane (10 mL), washed successively with water (5 mL), 1M HCl (5 mL), and water (5 mL), dried over anhydrous sodium sulphate (Na₂SO₄), filtered and concentrated to dryness to yield a residue. The residue was subjected to silica gel chromatography (0-10% MeOH/DCM) to yield dimethyl 6,6′-((2-(((5-isothiocyanatopentyl)thio)methyl)-1,4,10,13-tetraoxa-7,16-diazacyclooctadecane-7,16-diyl)bis(methylene))(S)-dipicolinate as a yellow solid (30 mg).

Step 3: A stir bar, dimethyl 6,6′-((2-(((5-isothiocyanatopentyl)thio)methyl)-1,4,10,13-tetraoxa-7,16-diazacyclooctadecane-7,16-diyl)bis(methylene))(S)-dipicolinate (30 mg, 0.040 mmol), and aqueous HCl (6 N, 0.2 mL, 0.8 mmol) were added to a 10 mL single-neck round-bottomed flask and stirred overnight at room temperature. The reaction mixture was concentrated to dryness in vacuo, and the concentrate was purified by HPLC (Column: XBRIDGE C18 19×150 mm, 5.0 μm; Mobile phase: 0.1% TFA in water/acetonitrile; Flow Rate: 15.0 mL/min) to yield (S)-6,6′-((2-(((5-isothiocyanatopentyl)thio)methyl)-1,4,10,13-tetraoxa-7,16-diazacyclooctadecane-7,16-diyl)bis(methylene))dipicolinic acid (12 mg). LC-MS APCI: Calculated for C₃₃H₄₇N₅O₈S₂: 705.29; Observed m/z [M+H]⁺ 706.2. ¹H NMR (400 MHz, DMSO-d₆): δ 13.40 (s, 1H), 9.90 (s, 1H), 8.17-8.09 (m, 4H), 7.78 (d, J=6.80 Hz, 2H), 4.70 (s, 4H), 3.93-3.17 (m, 27H), 2.68-2.67 (m, 2H), 1.64-1.60 (m, 2H), 1.53-1.49 (m, 2H), 1.40-1.38 (m, 2H).

### Example 8

### (S)-6,6′-((2-(((2-(2-(4-Isothiocyanatophenoxy)ethoxy)ethyl)thio)methyl)-1,4,10,13-tetraoxa-7,16-diazacyclooctadecane-7,16-diyl)bis(methylene))dipicolinic acid

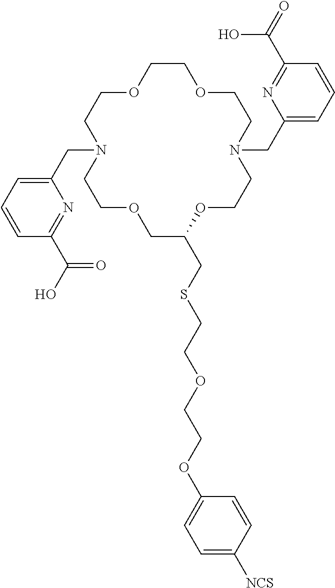

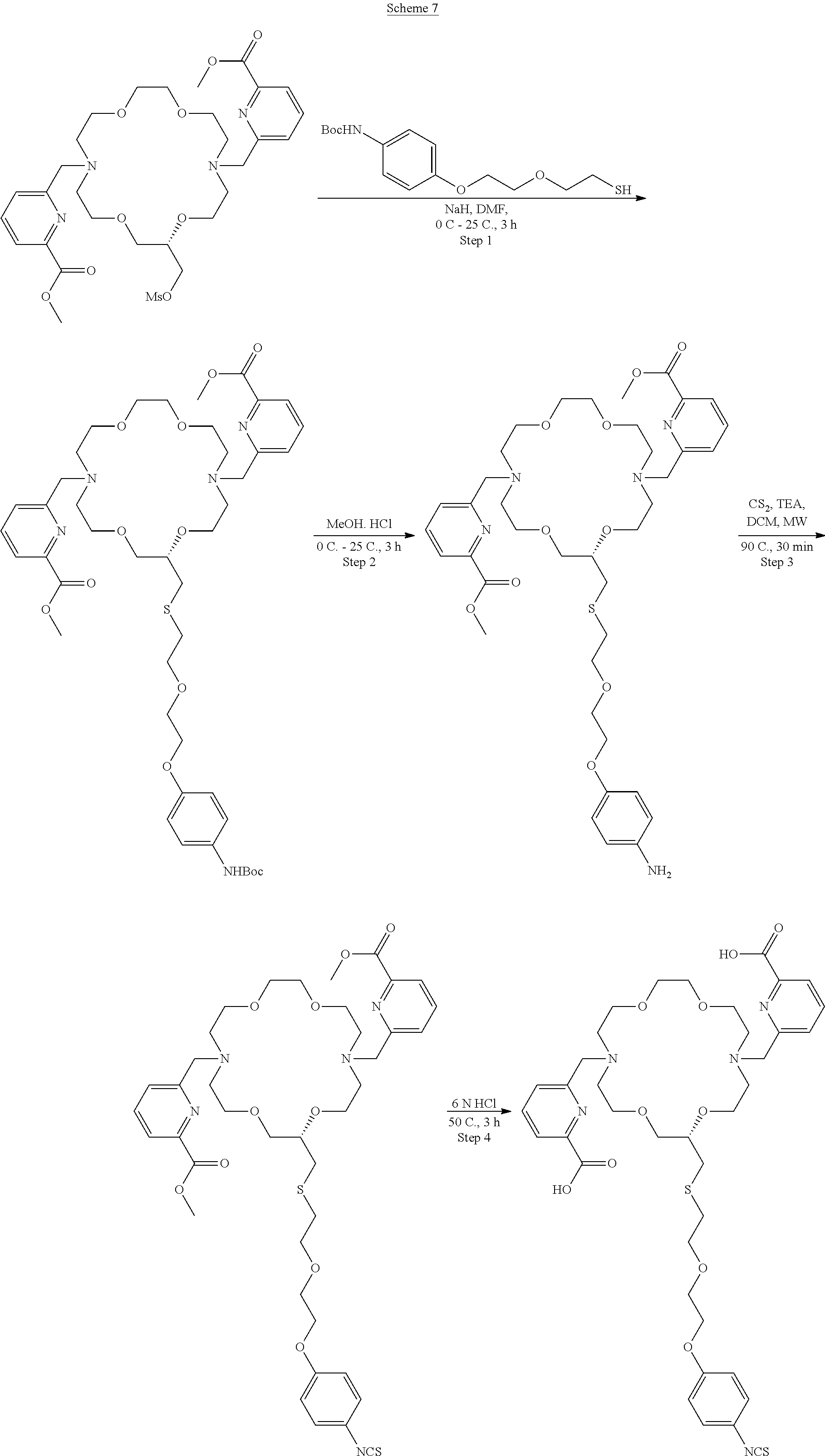

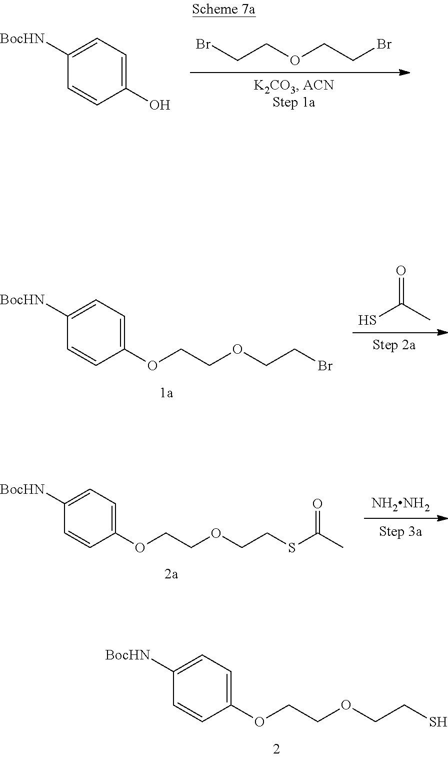

Scheme 7a, Step 1a: A stir bar, tert-butyl (4-hydroxyphenyl)carbamate (4.5 g, 22 mmol), 1-bromo-2-(2-bromoethoxy)ethane (5.0 g, 22 mmol), K₂CO₃ (4.6 g, 43 mmol) and ACN (45 mL) were added to a 250 mL three-neck round-bottomed flask under nitrogen atmosphere, and the resultant reaction mixture was heated at 80° C. for 16 h under nitrogen atmosphere. Reaction mixture was cooled to room temperature, filtered through Celite®, and concentrated to dryness in vacuo to yield a concentrate which was purified by silica gel chromatography (0-20% EtOAc/pet ether) to afford product tert-butyl (4-(2-(2-bromoethoxy)ethoxy)phenyl)carbamate (2.0 g).

Step 2a: A stir bar, tert-butyl (4-(2-(2-bromoethoxy)ethoxy)phenyl)carbamate (2.0 g, 5.6 mmol), ethanethioic S-acid (0.42 g, 5.6 mmol), K₂CO₃ (1.5 g, 11 mmol) and ACN (50 mL) were added to a 250 mL three-neck round-bottomed flask under nitrogen atmosphere. The reaction mixture stirred at 80° C. for 2 h, and then cooled to room temperature, filtered through Celite®, and concentrated to dryness in vacuo. The concentrate was purified using neutral alumina chromatography (0-50% EtOAc/pet ether) to yield S-(2-(2-(4-((tert-butoxycarbonyl)amino)phenoxy)ethoxy)ethyl) ethanethioate (1.8 g).

Step 3a: A stir bar, S-(2-(2-(4-((tert-butoxycarbonyl)amino)phenoxy)ethoxy)ethyl) ethanethioate (1.8 g, 5.1 mmol), ethanol (20 mL) and hydrazine monohydrate (0.24 g, 0.24 mL, 7.6 mmol) were added to a 250 mL single-neck round-bottomed flask under nitrogen, and stirred at 80° C. for 1 h. The reaction mixture was then cooled to room temperature and concentrated to dryness in vacuo, to yield a concentrate which was purified via silica gel chromatography (5-10% EtOAc/pet ether) to yield tert-butyl (4-(2-(2-mercaptoethoxy)ethoxy)phenyl)carbamate (0.5 g) as a colorless oil.

Scheme 7, Step 1: A solution consisting of tert-butyl (4-(2-(2-mercaptoethoxy)ethoxy)phenyl)carbamate (0.40 g, 1.0 mmol) and DMF (3.0 mL) was added dropwise over 5 minutes to a 50 mL three-neck round-bottomed flask containing a suspension of sodium hydride (0.060 g, 60% in mineral oil, 1.5 mmol) in DMF (3.0 mL) at 0° C. and a under nitrogen atmosphere. Once addition was complete, the reaction mixture was warmed to room temperature and stirred for 15 minutes. The mixture was re-cooled to 0° C. and a solution consisting of dimethyl 6,6′-((2-(((methylsulfonyl)oxy)methyl)-1,4,10,13-tetraoxa-7,16-diazacyclooctadecane-7,16-diyl)bis(methylene))(S)-dipicolinate (0.5 g, 0.7 mmol) and DMF (3.0 mL) was added dropwise. Once addition was complete, the reaction mixture was slowly warmed to room temperature and stirred 1.5 h. The reaction was slowly treated with sat. NH₄Cl (0.2 mL) and then concentrated to dryness to yield an oil. The oil was purified by preparative HPLC (Column: XBRIDGE C18 19×150 mm 5.0 μm; Mobile phase: 0.1% TFA in water/acetonitrile; Flow Rate: 15.0 mL/min) to yield dimethyl 6,6′-((2-(((2-(2-(4-((tert-butoxycarbonyl)amino)phenoxy)ethoxy)ethyl)thio)methyl)-1,4,10,13-tetraoxa-7,16-diazacyclooctadecane-7,16-diyl)bis(methylene))(S)-dipicolinate (0.15 g) as a brown oil.

Step 2: A stir bar, dimethyl 6,6′-((2-(((2-(2-(4-((tert-butoxycarbonyl)amino)phenoxy)ethoxy)ethyl)thio)methyl)-1,4,10,13-tetraoxa-7,16-diazacyclooctadecane-7,16-diyl)bis(methylene))(S)-dipicolinate (0.15 g, 0.16 mmol), MeOH (1.0 mL) and HCl in methanol (4 M, 0.80 mL, 3.2 mmol) were added to a 25 mL single-neck round-bottomed flask at 0° C. and then brought to room temperature. and stirred for 3 h. The volatiles were removed in vacuo to yield dimethyl 6,6′-((2-(((2-(2-(4-aminophenoxy)ethoxy)ethyl)thio)methyl)-1,4,10,13-tetraoxa-7,16-diazacyclooctadecane-7,16-diyl)bis(methylene))(S)-dipicolinate (0.12 g), which was used without purification.

Step 3: A stir bar, dimethyl 6,6′-((2-(((2-(2-(4-aminophenoxy)ethoxy)ethyl)thio)methyl)-1,4,10,13-tetraoxa-7,16-diazacyclooctadecane-7,16-diyl)bis(methylene))(S)-dipicolinate (0.12 g, 0.15 mmol), triethylamine (46 mg, 0.46 mmol), dry DCM (3 mL) and carbon disulfide (17 mg, 0.22 mmol) were added to a pressure vial at room temperature under nitrogen atmosphere. The reaction mixture was subjected to microwave-irradiation (150 W power) at 90° C. for 30 min. The reaction mixture was cooled to room temperature and was diluted with dichloromethane (10 mL), washed successively with water (5 mL), 1M HCl (5 mL), and water (5 mL), dried over anhydrous Na₂SO₄ and concentrated to dryness to yield dimethyl 6,6′-((2-(((2-(2-(4-isothiocyanatophenoxy)ethoxy)ethyl)thio)methyl)-1,4,10,13-tetraoxa-7,16-diazacyclooctadecane-7,16-diyl)bis(methylene))(S)-dipicolinate (0.12 g), which was used in the without purification.

Step 4: A stir bar, dimethyl 6,6′-((2-(((2-(2-(4-isothiocyanatophenoxy)ethoxy)ethyl)thio)methyl)-1,4,10,13-tetraoxa-7,16-diazacyclooctadecane-7,16-diyl)bis(methylene))(S)-dipicolinate (0.12 g, 0.15 mmol) and aqueous HCl (6 N, 0.51 mL, 3.1 mmol) were added to a 10 mL single-neck round-bottomed flask and stirred at 50° C. for 3 h. The reaction mixture was cooled to room temperature, concentrated to dryness in vacuo, and the concentrate was purified via preparative HPLC (Column: XBRIDGE C18 19×150 mm 5.0 μm; Mobile phase: 0.1% TFA in water/acetonitrile; Flow Rate: 15.0 mL/min) to yield (S)-6,6′-((2-(((2-(2-(4-isothiocyanatophenoxy)ethoxy)ethyl)thio)methyl)-1,4,10,13-tetraoxa-7,16-diazacyclooctadecane-7,16-diyl)bis(methylene))dipicolinic acid (40 mg, 37%). LC-MS APCI: Calculated for C₃₈H₄₉N₅O₁₀S₂: 799.29; Observed m/z [M+H]⁺ 799.9. ¹H NMR (400 MHz, CD₃OD): δ 8.23-8.20 (m, 2H), 8.15-8.09 (m, 2H), 7.74-7.71 (m, 2H), 7.19 (d, J=8.80 Hz, 2H), 6.92 (d, J=9.20 Hz, 2H), 4.81 (s, 2H), 4.77 (s, 2H), 4.09-4.11 (m, 4H), 3.92-3.95 (m, 6H), 3.79 (t, J=4.00 Hz, 3H), 3.66-3.71 (m, 16H), 2.70-2.76 (m, 4H).

### Example 9

### (S)-6,6′-((2-(((2-(2-(2-(4-Isothiocyanatophenoxy)ethoxy)ethoxy)ethyl)thio)methyl)-1,4,10,13-tetraoxa-7,16-diazacyclooctadecane-7,16-diyl)bis(methylene))dipicolinic acid

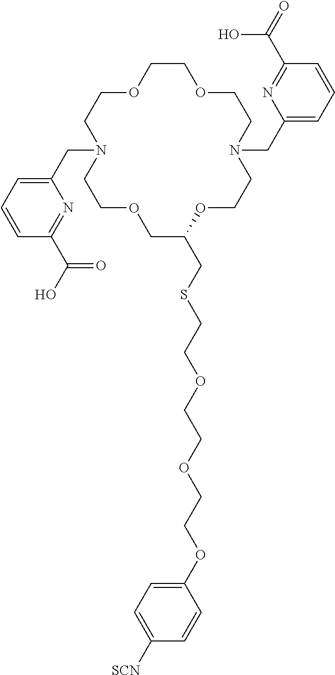

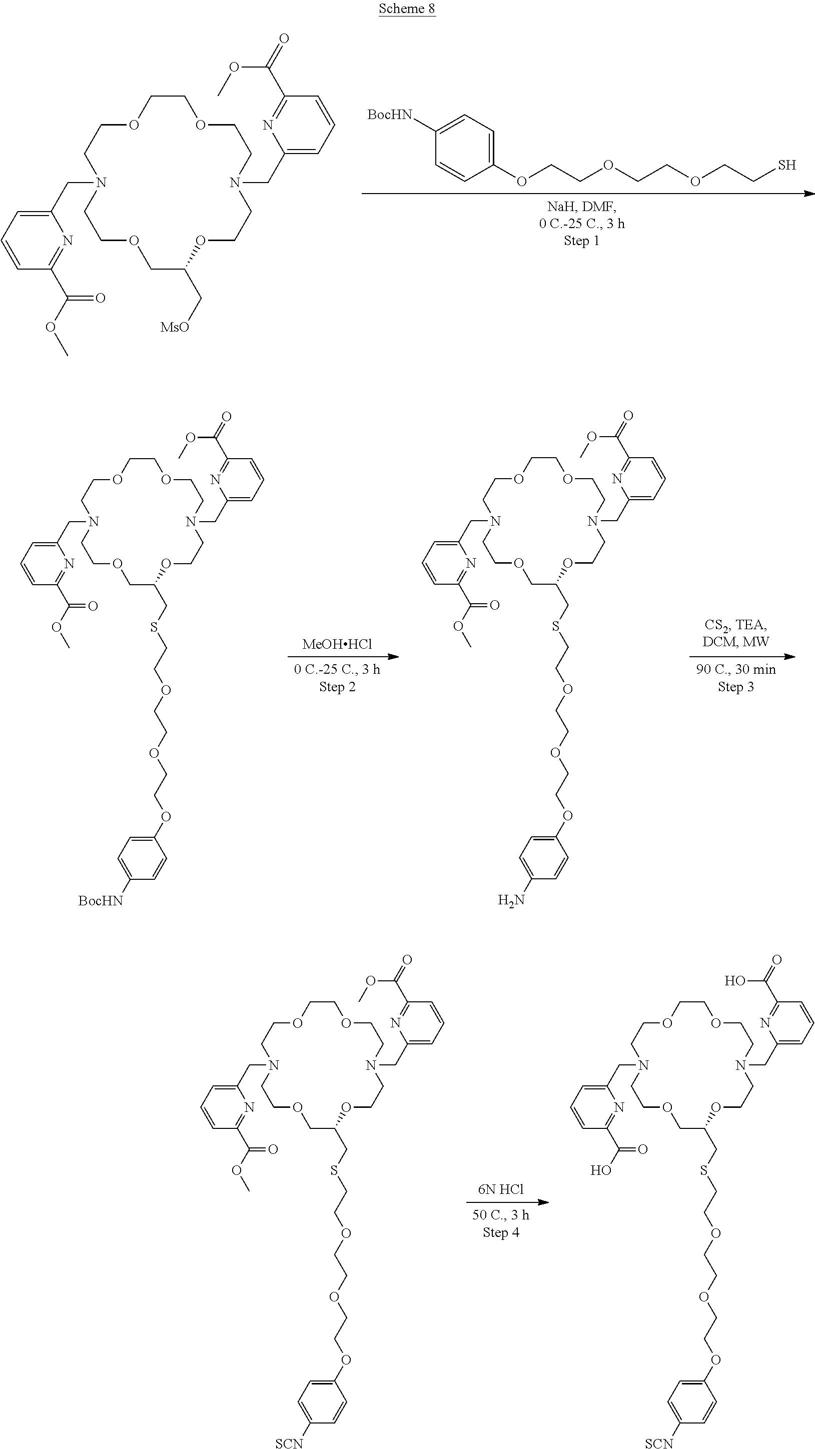

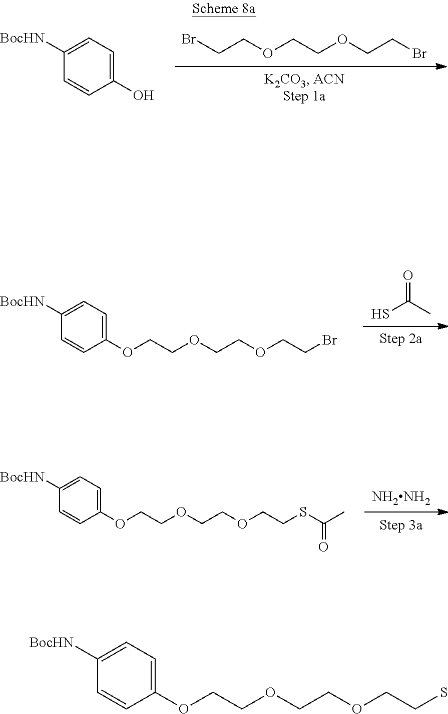

Scheme 8a, Step 1a: A stir bar, tert-butyl (4-hydroxyphenyl)carbamate (3.5 g, 17 mmol), 1,2-bis(2-bromoethoxy)ethane (4.6 g, 17 mmol), K₂CO₃ (4.6 g, 33 mmol) and ACN (40 mL) were added to a 250 mL three-neck round-bottomed flask, and then stirred at 80° C. for 48 h under a nitrogen atmosphere. The reaction mixture was cooled to room temperature, filtered through Celite® and concentrated to dryness in vacuo to yield a concentrate, which was purified via silica gel chromatography (0-10% MeOH/DCM) to yield tert-butyl (4-(2-(2-(2-bromoethoxy)ethoxy)ethoxy)phenyl)carbamate (4.0 g) as a brown oil.

Step 2a: A stir bar, tert-butyl (4-(2-(2-(2-bromoethoxy)ethoxy)ethoxy)phenyl)carbamate (4.0 g, 9.9 mmol), ethanethioic S-acid (0.75 g, 9.9 mmol), K₂CO₃ (2.7 g, 20 mmol) and ACN (50 mL) were added to a 250 mL three-neck round-bottomed flask under a nitrogen atmosphere, and the reaction mixture was heated at 60° C. for 2 h under a nitrogen atmosphere. The reaction mixture was cooled to room temperature, filtered through Celite®, concentrated to dryness in vacuo and the concentrate was purified by alumina chromatography (0-50% EtOAc/Pet Ether) to yield S-(2-(2-(2-(4-((tert-butoxycarbonyl)amino)phenoxy)ethoxy)ethoxy)ethyl) ethanethioate (3.0 g) as brown oil.

Step 3a: A stir bar, S-(2-(2-(2-(4-((tert-butoxycarbonyl)amino)phenoxy)ethoxy)ethoxy)ethyl) ethanethioate (3.0 g, 7.5 mmol), ethanol (50 mL) and hydrazine monohydrate (0.36 g, 0.36 mL, 11 mmol) were added to a 250 mL single-neck round-bottomed flask under nitrogen, and stirred at 80° C. for 1 h. The reaction mixture was cooled to room temperature, concentrated to dryness in vacuo, and the concentrate was purified by silica gel chromatography (5-10% EtOAc/pet ether) to yield tert-butyl (4-(2-(2-(2-mercaptoethoxy)ethoxy)ethoxy)phenyl)carbamate (1.0 g) as a colorless oil.

Scheme 7, Step 1: A solution consisting of tert-butyl (4-(2-(2-(2-mercaptoethoxy)ethoxy)ethoxy)phenyl)carbamate (0.40 g, 1.0 mmol) and DMF (3.0 mL) was added dropwise over 5 minutes to a 50 mL three-neck round-bottomed flask containing a suspension of sodium hydride (0.060 g, 60% in mineral oil, 1.5 mmol) in DMF (3.0 mL) at 0° C. under nitrogen atmosphere. Once addition was complete, the reaction mixture brought to room temperature and stirred continuously for 15 minutes. The mixture was re-cooled to 0° C. and a solution consisting of dimethyl 6,6′-((2-(((methylsulfonyl)oxy)methyl)-1,4,10,13-tetraoxa-7,16-diazacyclooctadecane-7,16-diyl)bis(methylene))(S)-dipicolinate (0.5 g, 0.7 mmol) and DMF (3.0 mL) was added dropwise over 10 minutes. Once addition was complete, the reaction mixture was slowly warmed to room temperature and stirred for 1.5 h. The reaction mixture was then slowly treated with sat. aqueous NH₄Cl (0.2 mL) and concentrated to dryness to yield an oil. The oil was purified by preparative HPLC (Column: XBRIDGE C18 (19×150 mm) 5.0 μm; Mobile phase: 0.1% TFA in water/acetonitrile; Flow Rate: 15.0 mL/min) to yield dimethyl 6,6′-((2-(((2-(2-(2-(4-((tert-butoxycarbonyl)amino)phenoxy)ethoxy)ethoxy)ethyl)thio)methyl)-1,4,10,13-tetraoxa-7,16-diazacyclooctadecane-7,16-diyl)bis(methylene))(S)-dipicolinate (0.15 g, 21%) as a brown oil.

Step 2: A stir bar, dimethyl 6,6′-((2-(((2-(2-(2-(4-((tert-butoxycarbonyl)amino)phenoxy)ethoxy)ethoxy)ethyl)thio)methyl)-1,4,10,13-tetraoxa-7,16-diazacyclooctadecane-7,16-diyl)bis(methylene))(S)-dipicolinate (0.15 mg, 0.16 mmol), MeOH (1.0 mL) and HCl in methanol (4 M, 0.80 mL, 3.2 mmol) were added to a 25 mL single-neck round-bottomed flask at 0° C. The reaction mixture was allowed to warm to room temperature and stirred for 3 h. The volatiles were removed in vacuo to give yield dimethyl 6,6′-((2-(((2-(2-(2-(4-aminophenoxy)ethoxy)ethoxy)ethyl)thio)methyl)-1,4,10,13-tetraoxa-7,16-diazacyclooctadecane-7,16-diyl)bis(methylene))(S)-dipicolinate (0.12 g), which was used in the next step without purification.

Step 3: A stir bar, dimethyl 6,6′-((2-(((2-(2-(2-(4-aminophenoxy)ethoxy)ethoxy)ethyl)thio)methyl)-1,4,10,13-tetraoxa-7,16-diazacyclooctadecane-7,16-diyl)bis(methylene))(S)-dipicolinate (0.12 g, 0.14 mmol), triethylamine (44 mg, 0.43 mmol) dry DCM (5 mL) and carbon disulfide (17 mg, 0.22 mmol) were added to a microwave vial at room temperature under a nitrogen atmosphere. The reaction mixture subjected to microwave irradiation (150 W power) at 90° C. for 30 min. The reaction mixture was then cooled to room temperature, diluted with dichloromethane (10 mL), washed successively with water (5 mL), 1M HCl (5 mL), and water (5 mL), dried over anhydrous Na₂SO₄ and concentrated to dryness to yield dimethyl 6,6′-((2-(((2-(2-(2-(4-isothiocyanatophenoxy)ethoxy)ethoxy)ethyl)thio)methyl)-1,4,10,13-tetraoxa-7,16-diazacyclooctadecane-7,16-diyl)bis(methylene))(S)-dipicolinate (0.12 g), which was used in the next step without purification.

Step 4: A stir bar, dimethyl 6,6′-((2-(((2-(2-(2-(4-isothiocyanatophenoxy)ethoxy)ethoxy)ethyl)thio)methyl)-1,4,10,13-tetraoxa-7,16-diazacyclooctadecane-7,16-diyl)bis(methylene))(S)-dipicolinate (0.12 mg, 0.14 mmol) and aqueous HCl (6 N, 0.50 mL, 2.8 mmol) were added to a 10 mL single-neck round-bottomed flask and stirred at 50° C. for 3 h. The reaction mixture was cooled to room temperature, concentrated to dryness in vacuo to yield a residue which was purified by preparative HPLC (Column: XBRIDGE C18 19×150 mm 5.0 μm; Mobile phase: 0.1% TFA in water/acetonitrile; Flow Rate: 15.0 mL/min) to yield (S)-6,6′-((2-(((2-(2-(2-(4-isothiocyanatophenoxy)ethoxy)ethoxy)ethyl)thio)methyl)-1,4,10,13-tetraoxa-7,16-diazacyclooctadecane-7,16-diyl)bis(methylene))dipicolinic acid (50 mg). LC-MS APCI: Calculated for C₄₀H₅₃N₅O₁₁S₂: 843.32; Observed m/z [M+H]⁺ 843.9. ¹H NMR (400 MHz, CD₃OD): δ 8.24-8.21 (m, 2H), 8.21-8.11 (m, 2H), 7.74 (d, J=7.60 Hz, 2H), 7.23-7.20 (m, 2H), 6.97-6.95 (m, 2H), 4.84-4.79 (m, 5H), 4.14-4.12 (m, 4H), 3.97-3.94 (m, 6H), 3.83-3.59 (m, 23H), 2.75-2.67 (m, 4H).

### Example 10

### 6-((4-((2-(2-(2-Aminoethoxy)ethoxy)ethyl)carbamoyl)phenyl)(16-((6-carboxypyridin-2-yl)methyl)-1,4,10,13-tetraoxa-7,16-diazacyclooctadecan-7-yl)methyl)picolinic acid

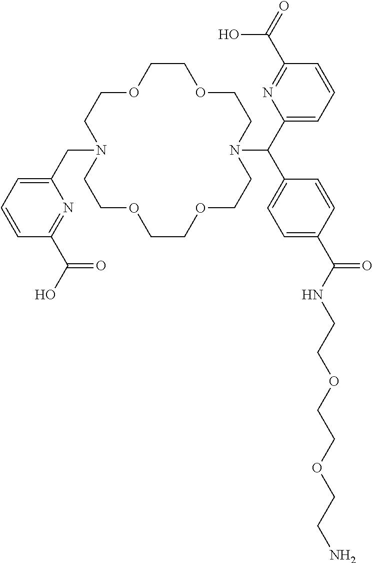 [SMILES: NCCOCCOCCNC(=O)c1ccc(C(c2cccc(C(=O)O)n2)N2CCOCCOCCN(Cc3cccc(C(=O)O)n3)CCOCCOCC2)cc1]

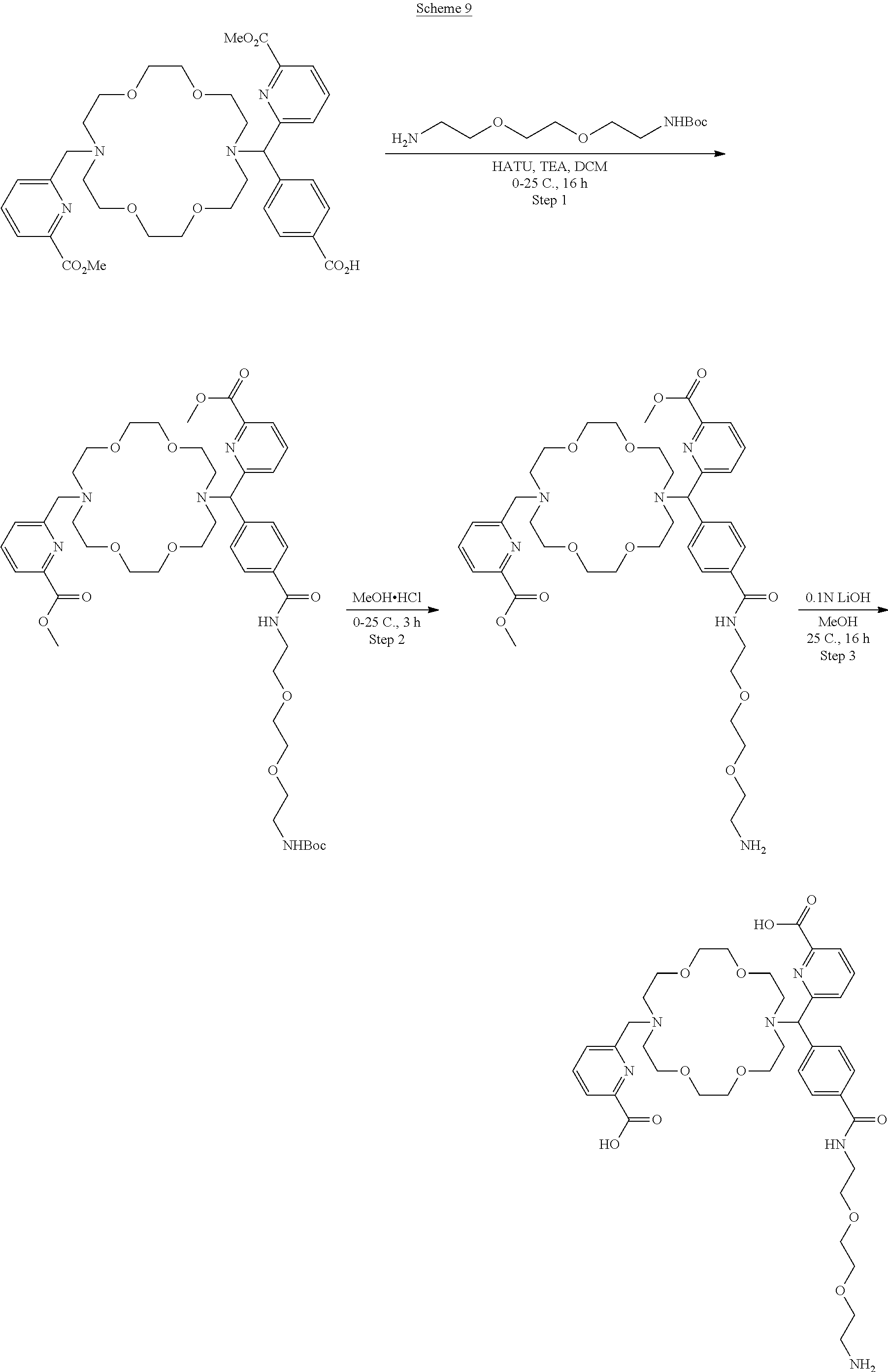

Step 1: A stir bar, 4-((6-(methoxycarbonyl)pyridin-2-yl)(16-((6-(methoxycarbonyl)pyridin-2-yl)methyl)-1,4,10,13-tetraoxa-7,16-diazacyclooctadecan-7-yl)methyl)benzoic acid (0.40 g, 0.60 mmol), tert-butyl (2-(2-(2-aminoethoxy)ethoxy)ethyl)carbamate (0.15 g, 0.60 mmol), triethylamine (0.18 g, 0.76 mmol), HATU (0.33 g, 0.90 mmol), and DCM (4.0 mL) were added to a 25 mL three-neck round-bottomed flask at 0° C. under a nitrogen atmosphere. The mixture was stirred overnight at room temperature and diluted with water (10 mL), and extracted with dichloromethane (10 mL×3). The combined extracts were washed with 10% aqueous NaHCO₃ (10 mL) and brine (10 mL), dried over anhydrous Na₂SO₄, filtered, and concentrated to dryness to yield a concentrate, which was purified via silica gel chromatography (0-10% MeOH/DCM) to yield methyl 6-((4-((2,2-dimethyl-4-oxo-3,8,11-trioxa-5-azatridecan-13-yl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.18 g).

Step 2: A stir bar, methyl 6-((4-((2,2-dimethyl-4-oxo-3,8,11-trioxa-5-azatridecan-13-yl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.18 g, 0.20 mmol), MeOH (1.8 mL), and HCl in methanol (4 M, 1.0 mL, 4.0 mmol) were added to a 10 mL single-neck round-bottomed flask at 0° C., and then brought to room temperature and stirred for 2 h. The volatiles were removed in vacuo to yield methyl 6-((4-((2-(2-(2-aminoethoxy)ethoxy)ethyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.15 g), which was used without purification.

Step 3: A stir bar, methyl 6-((4-((2-(2-(2-aminoethoxy)ethoxy)ethyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.1 g, 0.1 mmol), aqueous LiOH (3 mL, 0.1 N, 0.3 mmol), and MeOH (1.0 mL) were added to an 8 mL reaction vial at room temperature and stirred overnight. The reaction mixture was adjusted to pH-6.5 with acetic acid, and then concentrated to dryness in vacuo at room temperature to yield a concentrate, which was purified via preparative HPLC (Column: XBRIDGE C18 19×150 mm, 5.0 μm; Mobile phase: 0.1% TFA in water/ACN; Flow Rate: 15.0 mL/min) to yield 6-((4-((2-(2-(2-aminoethoxy)ethoxy)ethyl)carbamoyl)phenyl)(16-((6-carboxypyridin-2-yl)methyl)-1,4,10,13-tetraoxa-7,16-diazacyclooctadecan-7-yl)methyl)picolinic acid (40 mg). LC-MS APCI: Calculated for C₃₉H₅₄N₆O₁₁; 782.39; Observed m/z [M+H]+ 783.0.

### Example 11

### 6,6′-((18-(((2-(2-Aminoethoxy)ethyl)thio)methyl)tetradecahydro-4H,13H,17H-cyclopenta[b][1,4,10,13]tetraoxa[7,16]diazacyclooctadecine-4,13-diyl)bis(methylene))dipicolinic acid and

### Example 12

### N-acyl-DBCO tagged 6,6′-((18-(((2-(2-Aminoethoxy)ethyl)thio)methyl)tetradecahydro-4H,13H,17H-cyclopenta[b][1,4,10,13]tetraoxa[7,16]diazacyclooctadecine-4,13-diyl)bis(methylene))dipicolinic acid

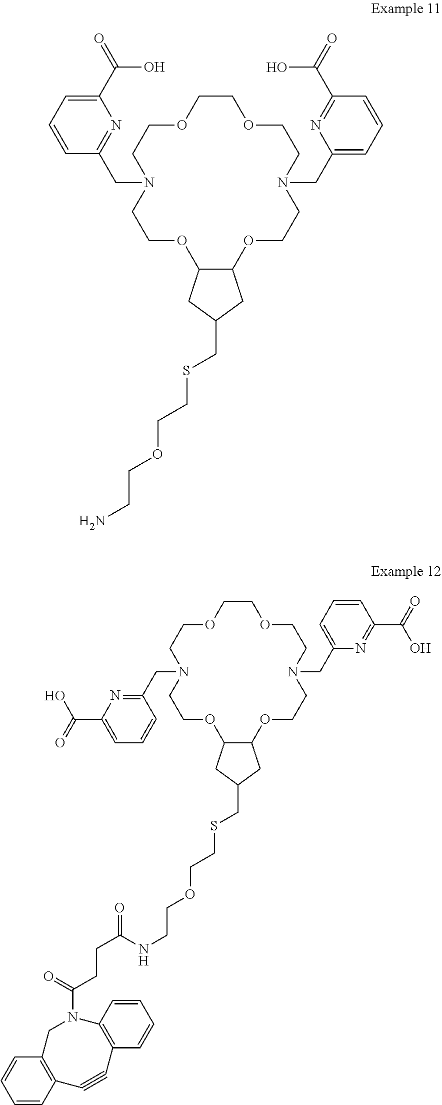 [SMILES: NCCOCCSCC1CC2OCCN(Cc3cccc(C(=O)O)n3)CCOCCOCCN(Cc3cccc(C(=O)O)n3)CCOC2C1 ; O=C(CCC(=O)N1Cc2ccccc2C#Cc2ccccc21)NCCOCCSCC1CC2OCCN(Cc3cccc(C(=O)O)n3)CCOCCOCCN(Cc3cccc(C(=O)O)n3)CCOC2C1]

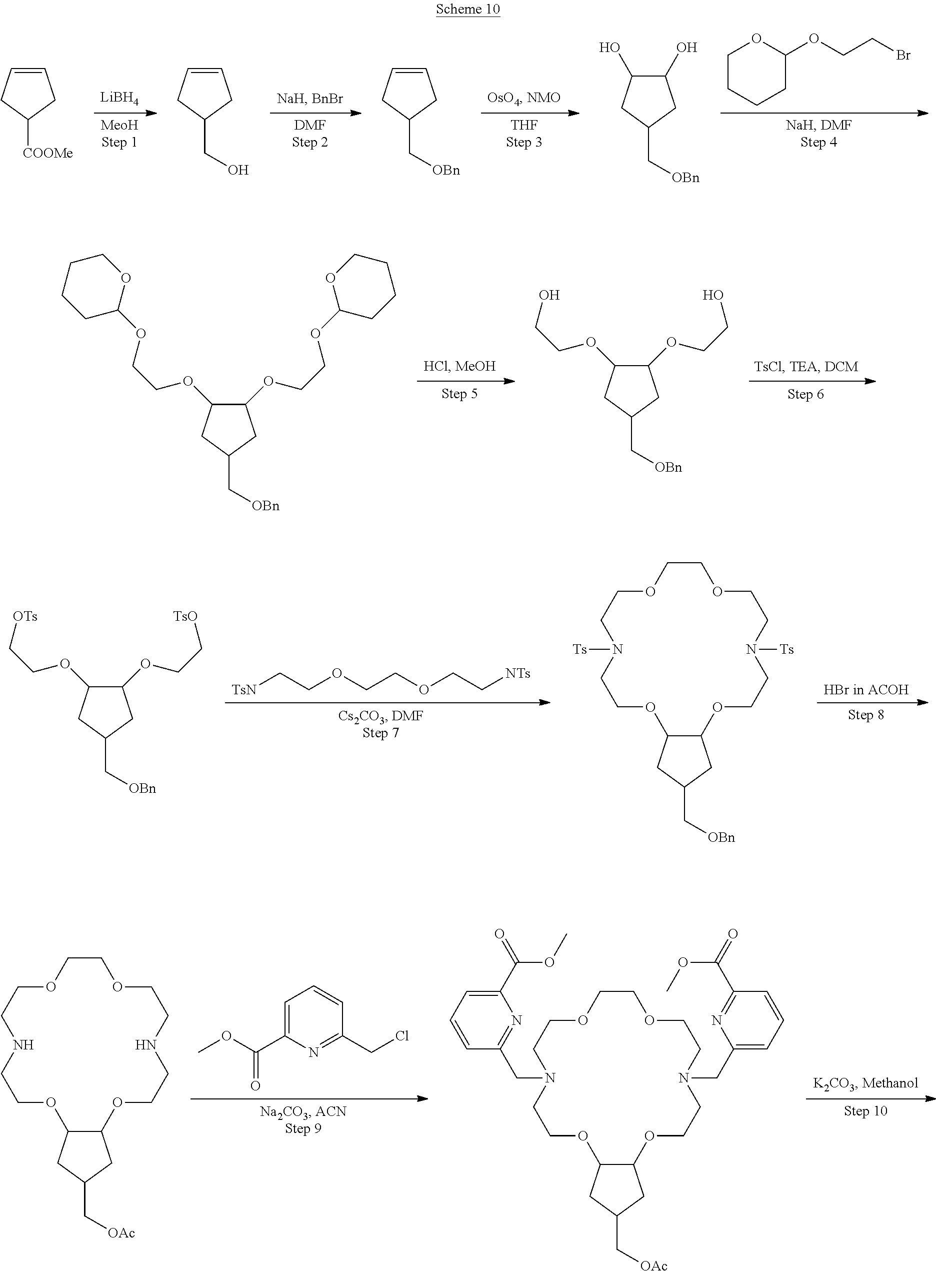

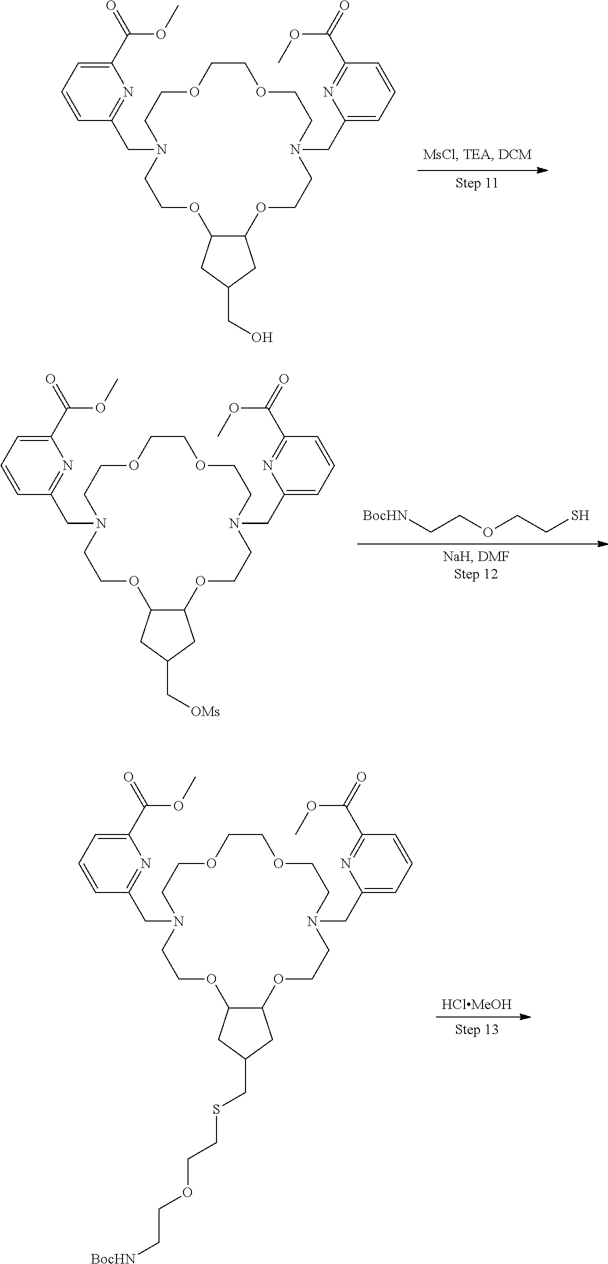

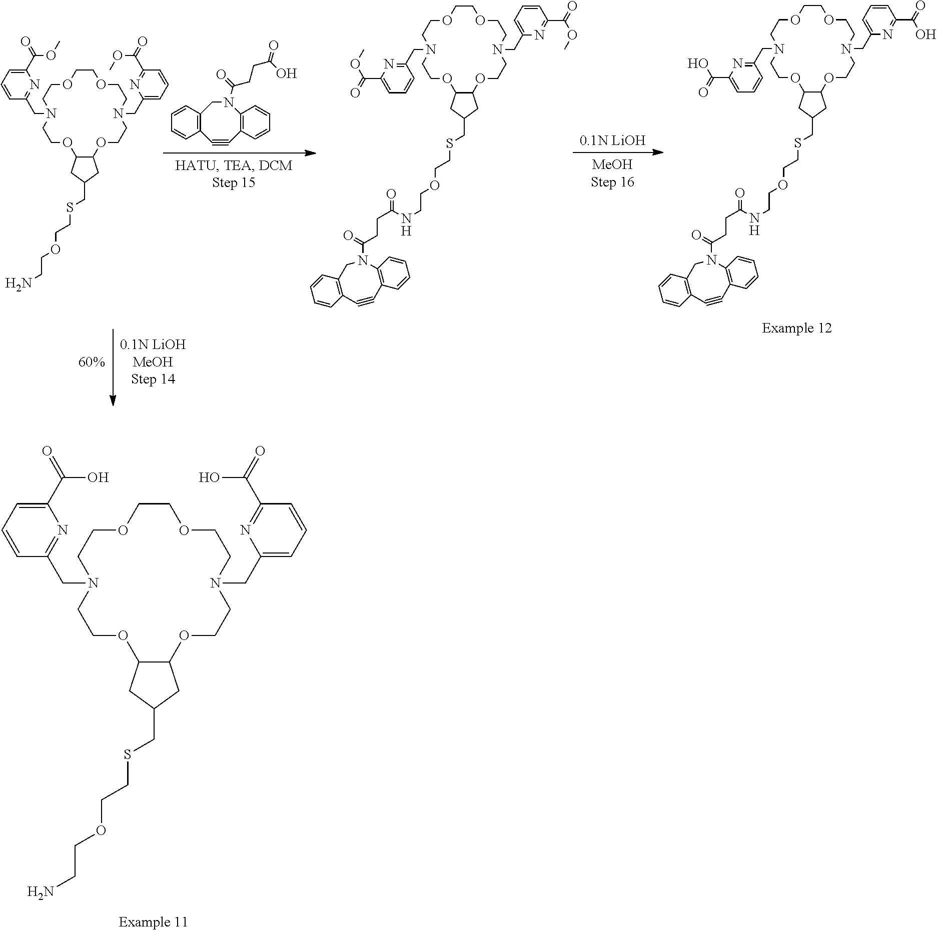

Step 1: A stir bar, methyl cyclopent-3-ene-1-carboxylate (25.0 g, 198 mmol), THF (600 mL), methanol (12.6 g, 16.0 mL, 397 mmol) and lithium borohydride (198 mL, 2.0 M in THF, 397 mmol) were added to a 3000 mL three-neck round-bottomed flask at 0° C. Once addition was complete, the reaction mixture was stirred at 70° C. for 6 h. The reaction mixture was then cooled to room temperature, slowly treated with ice water (250 mL), cooled further to 0° C., brought to pH-2 with 1.5 N HCl (pH-2) and then extracted with DCM (1000 mL×3). The combined extracts were washed with water (500 mL), dried over anhydrous Na₂SO₄, filtered and concentrated to dryness to yield a concentrate which was purified by silica gel chromatography (50-80% EtOAc/pet ether) to yield cyclopent-3-en-1-ylmethanol (13.8 g).

Step 2: A solution consisting of cyclopent-3-en-1-ylmethanol (13.7 g, 139 mmol) and DMF (50 mL) was added dropwise over 30 min into a 1000 mL three-neck round-bottomed flask containing a suspension of sodium hydride (6.69 g, 60% in mineral oil, 167 mmol) in DMF (50 mL) at 0° C. under nitrogen atmosphere. Once addition was complete, the reaction mixture was slowly warmed to room temperature and stirring continued for 30 min. The mixture was then re-cooled to 0° C. and treated dropwise over 15 min with a solution consisting of benzyl bromide (19.8 g, 167 mmol) and DMF (50 mL). Once addition was complete, the reaction mixture was slowly warmed to room temperature and then stirred for 16 h. The reaction mixture was slowly treated with sat. aqueous NH₄Cl (50 mL) and then extracted with ethyl acetate (1000 mL×3). The combined extracts were washed with water (500 mL×3), dried over anhydrous Na₂SO₄, filtered, and concentrated to dryness to yield a concentrate. The concentrate was purified by silica gel chromatography (0-20% EtOAc/pet ether) to yield ((cyclopent-3-en-1-ylmethoxy)methyl)benzene (21.0 g).

Step 3: A stir bar, NMO (38.0 g, 50% wt in H₂O, 158 mmol), THF (180 mL) and osmium tetroxide (16.2 g, 3.21 mL, 2.5% wt % in t-butanol, 0.158 mmol) were added to a 1000 mL three-neck round-bottomed flask at 0° C. The reaction mixture was brought to room temperature, stirred for 10 min and re-cooled to 0° C. Once cooled, the mixture was treated dropwise over 15 min with a solution of ((cyclopent-3-en-1-ylmethoxy)methyl)benzene (20.0 g, 158 mmol) and THF (180 mL). The reaction was brought to room temperature and stirred for 16 h before it was slowly treated with sat. aqueous NaHCO₃ (100 mL) and extracted with DCM (1000 mL×3). The combined extracts were washed with water (500 mL), dried over anhydrous Na₂SO₄, filtered, and concentrated to dryness to yield a concentrate, which was purified via silica gel chromatography (0-20% EtOAc/pet ether) to yield an isomeric mixture of 4-((benzyloxy)methyl)cyclopentane-1,2-diol as a colorless oil. The isomers were separated via SFC (Instrument: PIC 100; Column: Chiralpak OXH (250×30) mm, 5 μm; Mobile phase: CO₂: 0.5% isopropyl amine in IPA (60:40); Total flow: 70 g/min; Back pressure: 100 bar; Wave length: 220 nm; Cycle time: 8.0 min) yielded both cis-1,2 isomers of 4-((benzyloxy)methyl)cyclopentane-1,2-diol: 1^{st} eluting isomer (10 g) and 2^{nd} eluting isomer (5 g).

Step 4: A solution consisting of the 1^{st}-eluting isomer of 4-((benzyloxy)methyl)cyclopentane-1,2-diol (10.0 g, 45.0 mmol) and DMF (60 mL) was added dropwise over 1 h to a 250 mL three-neck round-bottomed flask containing a suspension of sodium hydride (8.62 g, 60% in mineral oil, 225 mmol) in DMF (60 mL) at 0° C. under a nitrogen atmosphere. Once addition was complete, the reaction mixture brought to room temperature and stirred for 30 min. The mixture was then re-cooled to 0° C. and treated dropwise over 15 min with a solution consisting of 2-(2-bromoethoxy)tetrahydro-2H-pyran (47.0 g, 225 mmol) and DMF (60 mL). Once addition was complete, the reaction mixture was slowly warmed to room temperature and stirred for 2 h. The mixture was then slowly treated with sat. aqueous NH₄Cl (50 mL) and then extracted with ethyl acetate (500 mL×3). The combined extracts were washed with water (500 mL), dried over anhydrous Na₂SO₄, filtered, and concentrated to dryness to yield an oil, which was purified by silica gel chromatography (0-30% EtOAc/pet ether) to yield 2,2′-((((4-((benzyloxy)methyl)cyclopentane-1,2-diyl)bis(oxy))bis(ethane-2,1-diyl))bis(oxy))bis(tetrahydro-2H-pyran) (21.0 g).

Step 5: A stir bar, 2,2′-((((4-((benzyloxy)methyl)cyclopentane-1,2-diyl)bis(oxy))bis(ethane-2,1-diyl))bis(oxy))bis(tetrahydro-2H-pyran) (29.0 g, 61.0 mmol), MeOH (200 mL) and HCl in 1,4-dioxane (4 M, 3.0 mL, 12.0 mmol) were added to a 1000 mL three-neck round-bottomed flask and then heated at reflux for 1 h. The flask was then cooled to room temperature and the volatiles removed in vacuo to yield 2,2′-((4-((benzyloxy)methyl)cyclopentane-1,2-diyl)bis(oxy))bis(ethan-1-ol) (20.0 g) as a residue, which was used without purification.

Step 6: A stir bar, 2,2′-((4-((benzyloxy)methyl)cyclopentane-1,2-diyl)bis(oxy))bis(ethan-1-ol) (20.0 g) (20.0 g, 64.4 mmol), DCM (200 mL) and triethylamine (32.6 mL, 322 mmol) were added to a 1000 mL round-bottomed flask under a nitrogen atmosphere, and the resulting mixture was cooled to 10° C. The mixture was then treated with pTsCl (36.9 g, 193 mmol) which was added portion-wise and then brought to room temperature. Once addition was complete the reaction mixture was stirred for 16 h during which time a precipitate formed. The mixture was then diluted with DCM (500 mL), washed with cold aq. HCl (1 M, 500 mL×3) and ice-cold water (500 mL×2), dried over anhydrous Na₂SO₄, filtered, and concentrated to dryness to yield a residue which was purified via silica gel chromatography (0-30% EtOAc/pet ether) to yield ((4-((benzyloxy)methyl)cyclopentane-1,2-diyl)bis(oxy))bis(ethane-2,1-diyl) bis(4-methylbenzenesulfonate) (26.0 g).

Step 7: A stir-bar, N,N′-((ethane-1,2-diylbis(oxy))bis(ethane-2,1-diyl))bis(4-methylbenzenesulfonamide) (21.0 g, 42.0 mmol), Cs₂CO₃ (41.3 g, 126 mmol) and dry DMF (250 mL) were added to a 2000 mL three-neck round-bottomed flask under nitrogen atmosphere, and the resultant heterogeneous mixture stirred at room temperature for 1.5 h. The mixture was then treated dropwise with a solution consisting of ((4-((benzyloxy)methyl)cyclopentane-1,2-diyl)bis(oxy))bis(ethane-2,1-diyl) bis(4-methylbenzenesulfonate) (26.0 g, 42.0 mmol) and DMF (250 mL) over a period of 2 h. Stirring was continued for 20 h, before the mixture was concentrated to dryness in vacuo to yield a paste-like solid. The paste was suspended in DCM (1000 mL), stirred for 30 min, and filtered by vacuum filtration. The filtrate was concentrated to dryness in vacuo to yield a concentrate, which was purified by silica gel chromatography (0-40% EtOAc/pet ether) to yield 18-((benzyloxy)methyl)-4,13-ditosyltetradecahydro-2H,11H,17H-cyclopenta[b][1,4,10,13]tetraoxa[7,16]diazacyclooctadecine (24 g).

Step 8: A HOAc solution of HBr (50%, 112 mL, 695 mmol) was added to a 500 mL round-bottomed flask containing a stir bar and 18-((benzyloxy)methyl)-4,13-ditosyltetradecahydro-2H,11H,17H-cyclopenta[b][1,4,10,13]tetraoxa[7,16]diazacyclooctadecine (24.0 g, 32.8 mmol) under a nitrogen atmosphere. The mixture was stirred at room temperature until homogeneous and then treated with phenol (16.3 g, 174 mmol). The reaction mixture was then heated at 60° C. for 6 h, before cooling to room temperature and concentrating to dryness in vacuo to yield a concentrate. The concentrate was purified via reverse-phase column chromatography (Column: Revelries C18-330 g; Mobile phase A: 0.1% TFA in water, Mobile phase B: acetonitrile; Flow rate: 60 mL/min) to yield (tetradecahydro-2H,11H,17H-cyclopenta[b][1,4,10,13]tetraoxa[7,16]diazacyclooctadecin-18-yl)methyl acetate (8.0 g).

Step 9: A stir bar, (tetradecahydro-2H,11H,17H-cyclopenta[b][1,4,10,13]tetraoxa[7,16]diazacyclooctadecin-18-yl)methyl acetate (8.0 g, 21 mmol), methyl 6-(chloromethyl)picolinate (12.2 g, 53.2 mmol), Na₂CO₃ (11.1 g, 106 mmol) and acetonitrile (100 mL) were added to a 500 mL three-neck round-bottomed flask under a nitrogen atmosphere, and the resultant heterogeneous mixture heated at 90° C. for 16 h under a nitrogen atmosphere. The resulting mixture was then cooled to room temperature, filtered through a pad of Celite®, and the filtrate concentrated to dryness in vacuo to yield a concentrate. The concentrate was subjected to silica gel chromatography (0-10% MeOH/DCM) to yield dimethyl 6,6′-((18-(acetoxymethyl)tetradecahydro-4H,13H,17H-cyclopenta[b][1,4,10,13]tetraoxa[7,16]diazacyclooctadecine-4,13-diyl)bis(methylene))dipicolinate (5.0 g).

Step 10: A stir bar, dimethyl 6,6′-((18-(acetoxymethyl)tetradecahydro-4H,13H,17H-cyclopenta[b][1,4,10,13]tetraoxa[7,16]diazacyclooctadecine-4,13-diyl)bis(methylene))dipicolinate (5.0 g, 7.4 mmol), K₂CO₃ (0.10 g, 0.74 mmol) and methanol (50 mL) were added to a 250 mL round-bottomed flask under nitrogen atmosphere, and the resulting mixture was stirred at room temperature for 10 min. The mixture was then concentrated to dryness in vacuo and the resulting residue purified by silica gel chromatography (0-10% MeOH/DCM) to yield 6,6′-((18-(hydroxymethyl)tetradecahydro-4H,13H,17H-cyclopenta[b][1,4,10,13]tetraoxa[7,16]diazacyclooctadecine-4,13-diyl)bis(methylene))dipicolinate (3.0 g).

Step 11: A stir bar, 6,6′-((18-(hydroxymethyl)tetradecahydro-4H,13H,17H-cyclopenta[b][1,4,10,13]tetraoxa[7,16]diazacyclooctadecine-4,13-diyl)bis(methylene))dipicolinate (2.0 g, 3.1 mmol), DCM (20 mL) and triethylamine (1.2 g, 9.5 mmol) were added to a 100 mL three-neck round-bottomed flask under a nitrogen atmosphere, and the resulting mixture cooled to 10° C. The mixture was treated with MsCl (0.48 g, 6.3 mmol) portion wise, and once addition was complete, the reaction vessel was brought to room temperature and stirred for 30 minutes, during which time a precipitate formed. The heterogeneous mixture was then diluted with DCM (50 mL), washed with cold aq. HCl (1 M, 50 mL×3) and ice-cold water (50 mL×2), dried over anhydrous Na₂SO₄, filtered, and concentrated to dryness to yield a gummy solid. The gummy solid was purified by neutral alumina column chromatography (0-10% MeOH/DCM) to yield dimethyl 6,6′-((18-(((methylsulfonyl)oxy)methyl)tetradecahydro-4H,13H,17H-cyclopenta[b][1,4,10,13]tetraoxa[7,16]diazacyclooctadecine-4,13-diyl)bis(methylene))dipicolinate (1.5 g).

Step 12: A solution consisting of dimethyl 6,6′-((18-(((methylsulfonyl)oxy)methyl)tetradecahydro-4H,13H,17H-cyclopenta[b][1,4,10,13]tetraoxa[7,16]diazacyclooctadecine-4,13-diyl)bis(methylene))dipicolinate (0.69 g, 3.2 mmol) and DMF (5 mL) was added dropwise over 5 minutes to a 25 mL three-neck round-bottomed flask containing a suspension of sodium hydride (162 mg, 60% in mineral oil, 4.22 mmol) in DMF (0.5 mL), at 0° C. under nitrogen atmosphere. Once addition was complete, the reaction mixture was brought to room temperature and stirred 15 minutes. The reaction mixture was then re-cooled to 0° C. and treated dropwise over 5 minutes with a solution consisting of tert-butyl (2-(2-mercaptoethoxy)ethyl)carbamate (1.50 g, 2.11 mmol) and DMF (3 mL). Once addition was complete, the reaction mixture was slowly warmed to room temperature and then stirred for 1 h. The reaction was then slowly treated with sat. NH₄Cl and subsequently extracted with ethyl acetate (10 mL×3). The combined extracts were washed with water (10 mL), dried over anhydrous Na₂SO₄, filtered, and concentrated to dryness to yield an oil. The oil was purified via preparative HPLC (Column: XBRIDGE C18 19×150 m) 5.0 μm; Mobile phase: 0.1% TFA in water/acetonitrile; Flow Rate: 15.0 mL/min) to yield cyclopenta[b][1,4,10,13]tetraoxa[7,16]diazacyclooctadecine-4,13-diyl)bis(methylene))dipicolinate (0.2 g).

Step 13: A stir bar, cyclopenta[b][1,4,10,13]tetraoxa[7,16]diazacyclooctadecine-4,13-diyl)bis(methylene))dipicolinate (0.20 g, 0.24 mmol), MeOH (1.0 mL), and HCl in methanol (4 M, 1.2 mL, 4.8 mmol) were added to a 25 mL single-neck round-bottomed flask at 0° C. and the resulting mixture brought to room temperature, and stirred for 2 h. The volatiles were removed in vacuo to yield dimethyl 6,6′-((18-(((2-(2-aminoethoxy)ethyl)thio)methyl)tetradecahydro-4H,13H,17H-cyclopenta[b][1,4,10,13]tetraoxa[7,16]diazacyclooctadecine-4,13-diyl)bis(methylene))dipicolinate (150 mg), which was used without purification.

Step 14: A stir bar, dimethyl 6,6′-((18-(((2-(2-aminoethoxy)ethyl)thio)methyl)tetradecahydro-4H,13H,17H-cyclopenta[b][1,4,10,13]tetraoxa[7,16]diazacyclooctadecine-4,13-diyl)bis(methylene))dipicolinate (40 mg, 0.054 mmol), aqueous LiOH (1.6 mL, 0.1 N, 0.16 mmol) and MeOH (0.5 mL) were added to an 8 mL reaction vial at room temperature and the resulting mixture was stirred overnight. The pH of the reaction mixture was adjusted with acetic acid to pH-6.5 and then concentrated to dryness in vacuo at room temperature, and the resultant concentrate was purified by preparative HPLC (Column: XBRIDGE C18 19×150 mm 5.0 μm; Mobile phase: 10 Mm Ammonium Acetate in water/ACN; Flow Rate: 15.0 mL/min) to yield Example 11: 6,6′-((18-(((2-(2-aminoethoxy)ethyl)thio)methyl)tetradecahydro-4H,13H,17H-cyclopenta[b][1,4,10,13]tetraoxa[7,16]diazacyclooctadecine-4,13-diyl)bis(methylene))dipicolinic acid (23 mg). LC-MS APCI: Calculated for C₃₄H₅₁N₅O₉S; 705.34; Observed m/z [M+H]⁺ 706.4.

Step 15: A stir bar, dimethyl 6,6′-((18-(((2-(2-aminoethoxy)ethyl)thio)methyl)tetradecahydro-4H,13H,17H-cyclopenta[b][1,4,10,13]tetraoxa[7,16]diazacyclooctadecine-4,13-diyl)bis(methylene))dipicolinate (70 mg, 0.95 mmol), 11,12-Didehydro-γ-oxodibenz[b,f]azocine-5(6H)-butanoic acid (29 mg, 0.95 mmol), triethylamine (29 mg, 0.76 mmol), HATU (54 mg, 0.14 mmol) and DCM (0.5 mL) were added to a 25 mL three-neck round-bottomed flask at 0° C. under a nitrogen atmosphere. The resulting mixture was brought to room temperature and stirred overnight. The reaction mixture was diluted with water (10 mL) and the extracted with dichloromethane (10 mL×3). The combined extracts were washed with 10% aqueous NaHCO₃ (10 mL) and brine (10 mL), dried over anhydrous Na₂SO₄, filtered, and concentrated to dryness to yield an oil. The oil was purified via silica gel chromatography (0-10% MeOH/DCM) to yield N-acyl-DBCO tagged dimethyl 6,6′-((18-(((2-(2-aminoethoxy)ethyl)thio)methyl)tetradecahydro-4H,13H,17H-cyclopenta[b][1,4,10,13]tetraoxa[7,16]diazacyclooctadecine-4,13-diyl)bis(methylene))dipicolinate (10 mg).

Step 16: A stir bar, N-acyl-DBCO tagged dimethyl 6,6′-((18-(((2-(2-aminoethoxy)ethyl)thio)methyl)tetradecahydro-4H,13H,17H-cyclopenta[b][1,4,10,13]tetraoxa[7,16]diazacyclooctadecine-4,13-diyl)bis(methylene))dipicolinate (10 mg, 0.01 mmol), aqueous LiOH (0.3 mL, 0.1 N, 0.03 mmol) and methanol (0.25 mL) were added to an 8 mL reaction vial at room temperature and the resultant mixture stirred overnight. The reaction mixture was adjusted to pH˜6.5 with acetic acid, concentrated to dryness in vacuo at room temperature, and the resultant concentrate was purified by preparative HPLC (Column: XBRIDGE C18 (19×150 mm) 5.0 μm; Mobile phase: 10 Mm Ammonium Acetate in water/ACN; Flow Rate: 15.0 mL/min) to yield

Example 12: N-acyl-DBCO tagged 6,6′-((18-(((2-(2-aminoethoxy)ethyl)thio)methyl)tetradecahydro-4H,13H,17H-cyclopenta[b][1,4,10,13]tetraoxa[7,16]diazacyclooctadecine-4,13-diyl)bis(methylene))dipicolinic acid (3 mg). LC-MS APCI: Calculated for C₅₃H₆₄N₆O₁₁S; 992.44; Observed m/z [M−H]⁻: 991.4.

### Example 13

### 6-((16-(1-(6-carboxypyridin-2-yl)-8-isothiocyanatooctyl)-1,4,10,13-tetraoxa-7,16-diazacyclooctadecan-7-yl)methyl)picolinic acid

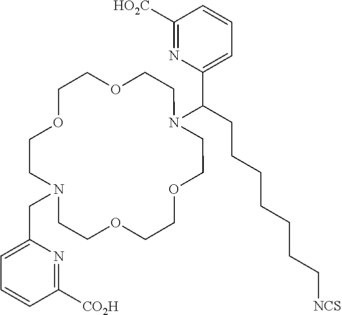

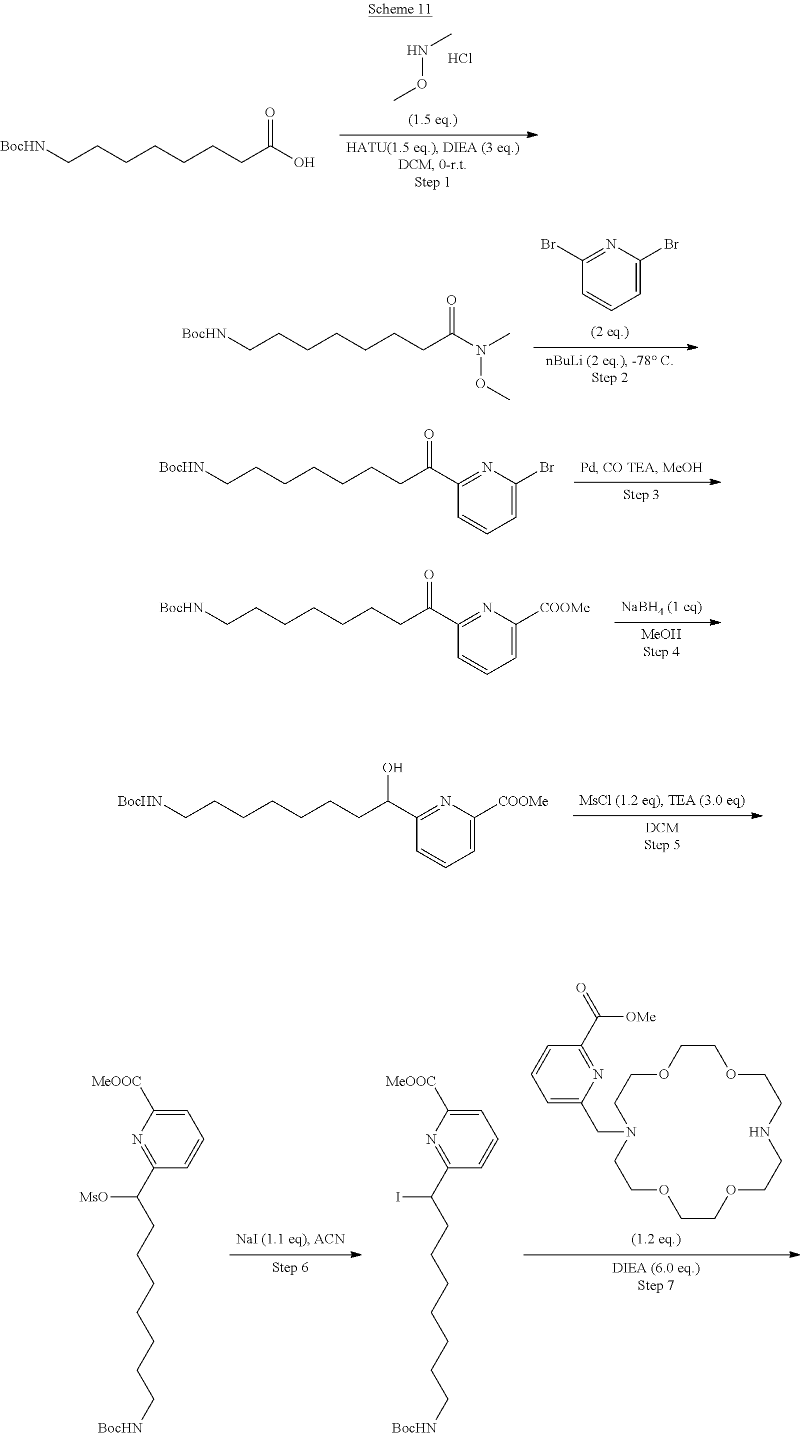

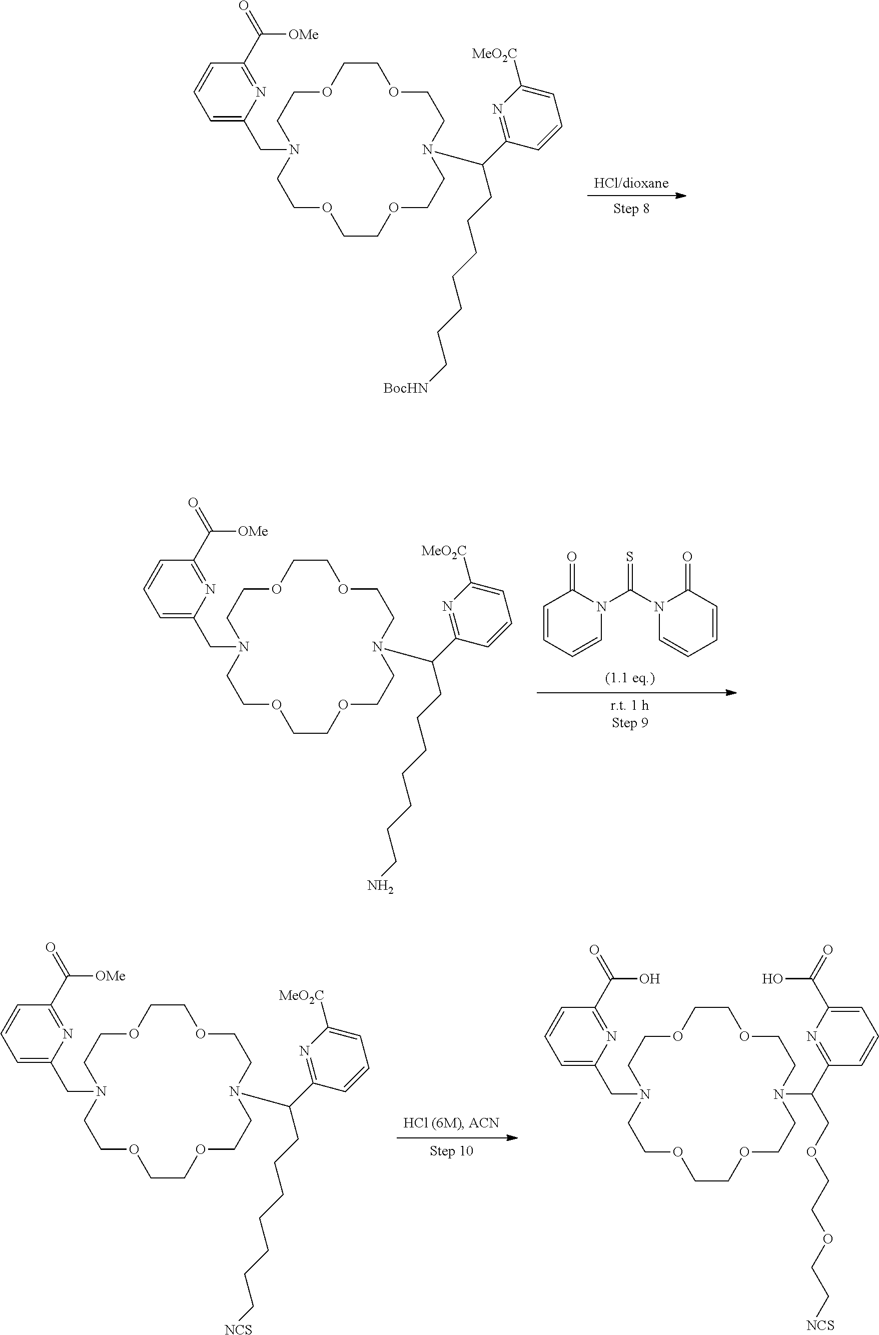

Step 1: Into a 500-mL 3-necked round-bottom flask, purged and maintained under an inert atmosphere of nitrogen, was placed a solution of 8-((tert-butoxycarbonyl)amino)octanoic acid (20.0 g, 77.1 mmol) in dichloromethane (200 mL), N, O-dimethylhydroxylamine (7.0 g, 115 mmol), diisopropylethylamine (29.90 g, 231 mmol). This was followed by the addition of HATU (43.9 g, 115 mmol) with stirring at 0° C. The resulting solution was stirred for 1 h. at room temperature. The reaction was then quenched by the addition of 200 mL of water. The resulting solution was extracted with dichloromethane (100 mL×2). The combined organic layers were washed sequentially with HCl (1 M) (300 mL×2), NH₄CO₃ aqueous solution (400 mL×3) and bine (400 mL). After it was dried over anhydrous Na₂SO₄, it was concentrated to give tert-butyl (8-(methoxy(methyl)amino)-8-oxooctyl)carbamate (15.4 g, 66% yield) as light-yellow oil.

Step 2: Into a 500-mL 3-necked round-bottom flask, purged and maintained under an inert atmosphere of nitrogen, was placed a solution of 2,6-dibromopyridine (23.0 g, 927 mmol) in THF (400 mL). It was cooled to −78° C. and n-BuLi (60.4 mL, 927 mmol) was added dropwise quickly. After stirring for 10 min, an addition of tert-butyl (8-(methoxy(methyl)amino)-8-oxooctyl)carbamate (14.0 g, 463.5 mmol) in THF (40 mL) was added dropwise with stirring at −78° C. The resulting solution was stirred for 30 min. at room temperature. The reaction was quenched by the addition of 500 mL of water. The resulting solution was extracted with ethyl acetate (200 mL×2). The combined organic layers were washed with brine (400 mL), dried over anhydrous sodium sulfate and concentrated to give the crude product. Chromatography on silica gel ((0-10% ethyl acetate in petroleum ether) gave tert-butyl (8-(6-bromopyridin-2-yl)-8-oxooctyl)carbamate (11.8 g, 50% yield) as light yellow solid.

Step 3: Into a 1-L high pressure reactor, maintained with an inert atmosphere of nitrogen, was placed a solution of tert-butyl (8-(6-bromopyridin-2-yl)-8-oxooctyl)carbamate (11.5 g, 28.8 mmol, 1.0 eq.) in MeOH (500 mL), followed by Pd(dppf)Cl₂ (2.1 g, 2.88 mmol), TEA (8.7 g, 86.4 mmol). Then CO (20 atm) was introduced in. The resulting solution was stirred for 16 h at 100° C. The reaction solution was filtered and used for next step directly.

Step 4: The MeOH solution received from above was cooled to 0° C. and NaBH₄ (1.08 g, 28.8 mmol) was added. The resulting solution was stirred for 1 h. at room temperature. The reaction was quenched by the addition of 500 mL of NH₄CO₃ aqueous solution and extracted with ethyl acetate (300 mL×2). The combined organic layers were washed with brine (600 mL), dried over anhydrous Na₂SO₄ and concentrated to give methyl 6-(8-((tert-butoxycarbonyl)amino)-1-hydroxyoctyl)picolinate (10 g) as brown oil.

Step 5: Into a 250-mL 3-necked round-bottom flask, purged and maintained under an inert atmosphere of nitrogen, was placed a solution of methyl 6-(8-((tert-butoxycarbonyl)amino)-1-hydroxyoctyl)picolinate (10 g) in DCM (100 mL). After it was cooled to 0° C., TEA (7.9 g, 78.9 mmol) and mesyl chloride (3.6 g, 31.5 mmol) were added. The resulting solution was stirred for 1 h. at room temperature. The mixture was concentrated under vacuum. MeCN (100 mL) was added and concentrated under vacuum. The crude product methyl 6-(8-((tert-butoxycarbonyl)amino)-1-((methylsulfonyl)oxy)octyl)picolinate went straight to the next step.

Step 6: To a solution of the above crude product methyl 6-(8-((tert-butoxycarbonyl)amino)-1-((methylsulfonyl)oxy)octyl)picolinate in ACN (100 mL) was added NaI (4.3 g, 28.9 mmol). The resulting solution was stirred for 1 h at 80° C. The mixture was filtered and concentrated. The crude product was purified by Flash-Prep-HPLC: Column C18; mobile phase, H₂O/ACN=50/50% to H₂O/ACN=20/80% in 30 min; It gave 4 g of methyl 6-(8-((tert-butoxycarbonyl)amino)-1-iodooctyl)picolinate as brown oil.

Step 7: To a solution of methyl 6-(8-((tert-butoxycarbonyl)amino)-1-iodooctyl)picolinate (3.0 g, 6.12 mmol,) in DCM (200 mL) were added methyl 6-((1,4,10,13-tetraoxa-7,16-diazacyclooctadecan-7-yl)methyl)picolinate (3.0 g, 7.34 mmol), diisopropylethylamine (3.9 g, 30.61 mmol). The resulting solution was stirred for 16 h at 80° C. The reaction was concentrated. The crude product was purified by Flash-Prep-HPLC: Column C18; mobile phase, A: H₂O (0.05% TFA), B: CAN; 20% B to 40% B in 20 min. It gave 1.9 g of methyl 6-(8-((tert-butoxycarbonyl)amino)-1-(16-((6-(methoxycarbonyl)pyridin-2-yl)methyl)-1,4,10,13-tetraoxa-7,16-diazacyclooctadecan-7-yl)octyl)picolinate as brown oil.

Step 8: To a stirred solution of methyl 6-(8-((tert-butoxycarbonyl)amino)-1-(16-((6-(methoxycarbonyl)pyridin-2-yl)methyl)-1,4,10,13-tetraoxa-7,16-diazacyclooctadecan-7-yl)octyl)picolinate (1.7 g, 2.19 mmol, 77% on LCMS) in DCM (8.5 mL) at 0° C. was added HCl/dioxane dropwise. The resulting solution was stirred for 1 h. at room temperature. The reaction was quenched by the portion wise addition of NH₄CO₃ aqueous solution (20 mL×3). The resulting solution was extracted with dichloromethane (100 mL×2). The combined organic layers were washed brine (400 mL), dried over anhydrous Na₂SO₄ and concentrated to give 1.3 g of methyl 6-(8-amino-1-(16-((6-(methoxycarbonyl)pyridin-2-yl)methyl)-1,4,10,13-tetraoxa-7,16-diazacyclooctadecan-7-yl)octyl)picolinate as brown oil.

Step 9: To a solution of methyl 6-(8-amino-1-(16-((6-(methoxycarbonyl)pyridin-2-yl)methyl)-1,4,10,13-tetraoxa-7,16-diazacyclooctadecan-7-yl)octyl)picolinate (1.0 g, 1.48 mmol) in DCM (17 mL) under N₂ was added 1,1′-thiocarbonylbis(pyridin-2(1H)-one) (0.38 g, 1.63 mmol). The resulting solution was stirred for 1 h at room temperature. It was concentrated to give 1.6 g of methyl 6-((16-(1-(6-(methoxycarbonyl)pyridin-2-yl)-8-thiocyanatooctyl)-1,4,10,13-tetraoxa-7,16-diazacyclooctadecan-7-yl)methyl)picolinate as brown oil.

Step 10: To a solution of methyl 6-((16-(1-(6-(methoxycarbonyl)pyridin-2-yl)-8-thiocyanatooctyl)-1,4,10,13-tetraoxa-7,16-diazacyclooctadecan-7-yl)methyl)picolinate (1.40 g, 1.42 mmol) in ACN (4 mL) was added HCl (6 M) (7 mL). The resulting solution was stirred for 5 h at 50° C. in an oil bath. It was diluted with 10 mL of H₂O. The crude product was purified by Flash-Prep-HPLC: Column, C18; mobile phase, A: H₂O (0.05% TFA), B: ACN, 20% B to 36% B in 20 min; Detector UV@210 nm. The product fractions were concentrated to remove ACN. The aqueous was adjust to pH to 7-8 with NaHCO₃ aqueous solution. It was purified again on Flash-Prep-HPLC: Column, C18; mobile phase, A: H₂O, B: ACN, 95% B to 100% B in 20 min. The product solution was concentrated to remove CAN and then lyophilized. It gave 190 mg of 6-((16-(1-(6-carboxypyridin-2-yl)-8-thiocyanatooctyl)-1,4,10,13-tetraoxa-7,16-diazacyclooctadecan-7-yl)methyl)picolinic acid as a brown solid. ¹H NMR (300 MHz, D₂O) δ 7.91 (s, 4H), 7.54 (s, 2H), 4.52 (d, J=17.9 Hz, 3H), 3.77 (d, J=9.3 Hz, 8H), 3.56-3.41 (m, 18H), 2.11 (s, 2H), 1.51 (s, 2H), 1.17 (s, 7H), 0.97 (s, 1H). MS (ES, m/z): 688.3 (M+H⁺).

### Example 14

### 6-((16-(1-(6-carboxypyridin-2-yl)-2-(2-(2-isothiocyanatoethoxy)ethoxy)ethyl)-1,4,10,13-tetraoxa-7,16-diazacyclooctadecan-7-yl)methyl)picolinic acid

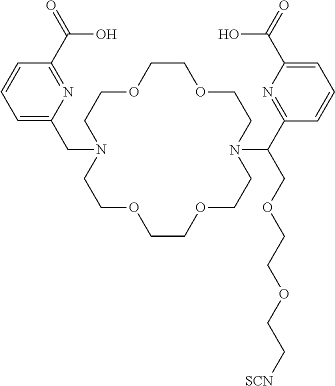

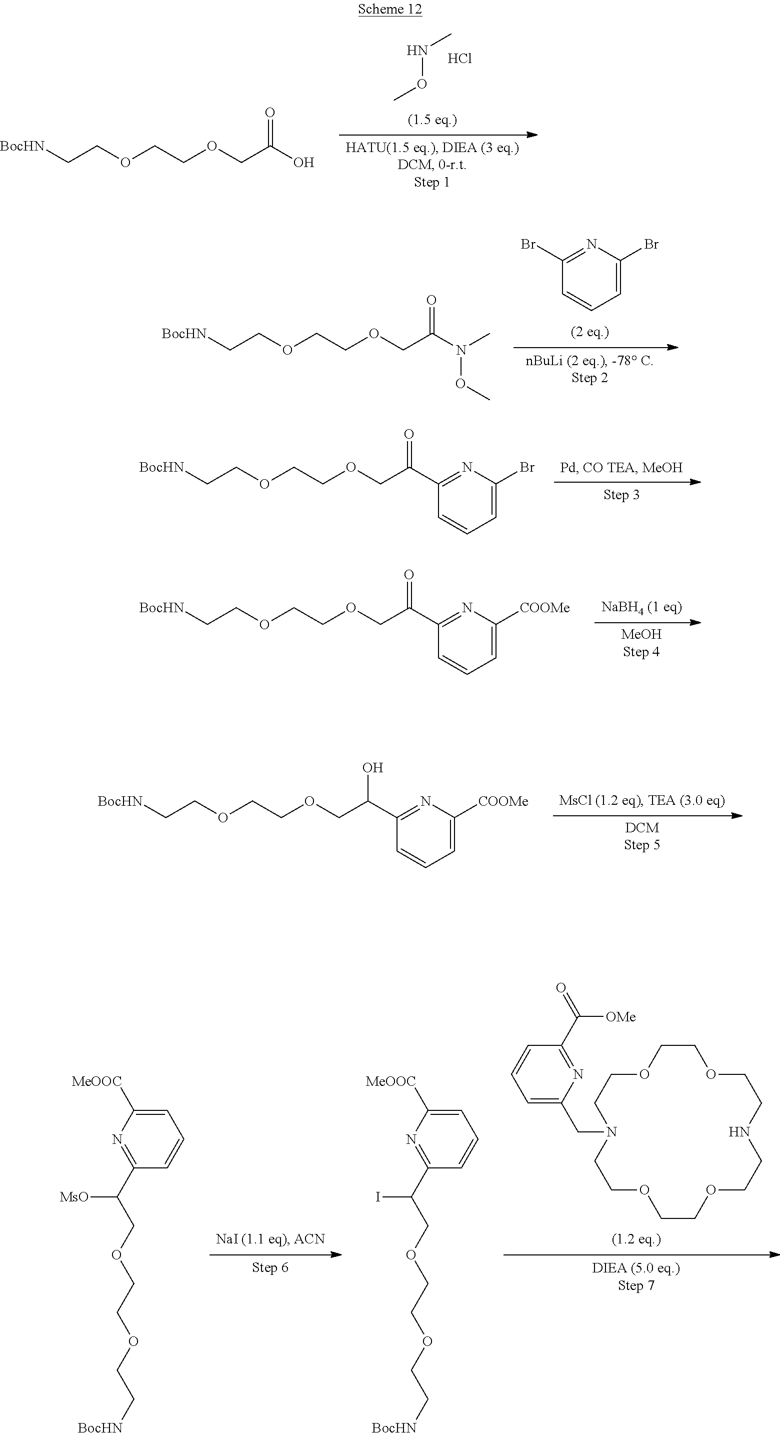

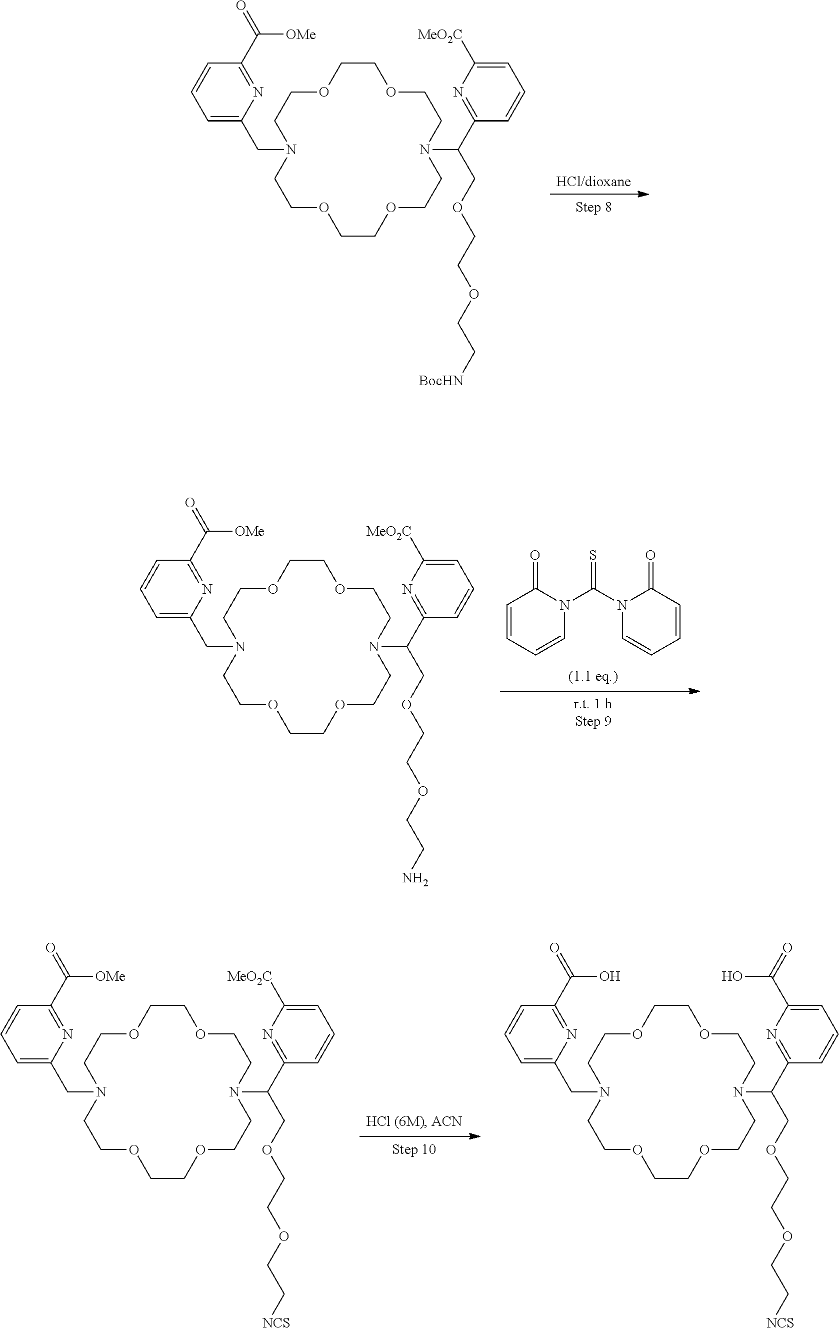

Step 1: To a stirred solution of 2,2-dimethyl-4-oxo-3,8,11-trioxa-5-azatridecan-13-oic acid (10.00 g, 37.98 mmol) and diisopropylethylamine (14.73 g, 113.94 mmol) in dichloromethane (100 mL) at 0° C. under nitrogen atmosphere was added [Bis(dimethylamino)methylene]-1H-1,2,3-triazolo[4,5-b]pyridinium 3-oxide hexafluorophosphate (15.16 g, 39.88 mmol), N,O-dimethyl hydroxylamine (5.55 g, 56.97 mmol) dropwise. After the resulting mixture was stirred for 1 h at room temperature, it was poured to saturated NH₄Cl (aq.). The resulting mixture was extracted with dichloromethane (100 mL×2). The combined organic layers were washed with brine, dried over anhydrous Na₂SO₄. After filtration, the filtrate was concentrated under reduced pressure. The residue was purified by chromatography: Column, C18; mobile phase A: H₂O with 0.05% TFA, B: ACN; gradient 20% B to 40% B in 20 minutes; Detector: UV@210 nm. It gave tert-butyl (3-methyl-4-oxo-2,6,9-trioxa-3-azaundecan-11-yl)carbamate (9.90 g. 85% yield) as light yellow oil.

Step 2: To a solution of 2,6-dibromo-pyridine (13.3 g, 56.1 mmol) in THF (260 mL) in a 500 ml 3-necked round-bottom flask at −78° C. under nitrogen atmosphere was added n-BuLi (28.0 mL, 56.1 mmol) dropwise. The solution was stirred at −78° C. for 10 min. A solution of tert-butyl (3-methyl-4-oxo-2,6,9-trioxa-3-azaundecan-11-yl)carbamate (7.0 g, 28.0 mmol) in THF (30 mL) was added dropwise to the reaction solution at −78° C. and the mixture was stirred at room temperature for 30 min. The reaction was quenched by the addition of water/ice (200 mL) at 0° C. The aqueous layer was extracted with ethyl acetate (100 mL×3). The combined extracts were dried over Na₂SO₄ and concentrated under vacuum. The residue was purified by chromatography: Column, C18; mobile phase, mobile phase A: H₂O with 0.05% TFA, B: ACN; gradient 38% B to 58% B in 20 minutes; Detector: UV@210 nm. It gave to tert-butyl (2-(2-(2-(6-bromopyridin-2-yl)-2-oxoethoxy)ethoxy)ethyl)carbamate (4.6 g, 50% yield) as a yellow solid. MS (ES, m/z): 425, 427 (M+Na⁺).

Step 3: To a 250-mL high pressure reactor were added tert-butyl (2-(2-(2-(6-bromopyridin-2-yl)-2-oxoethoxy)ethoxy)ethyl)carbamate (4.0 g, 18.1 mmol), triethylamine (5.5 g, 54.3 mmol), Pd(dppf)Cl₂ (1.3 g, 1.8 mmol) and MeOH (40 mL). The reaction solution was evacuated and backfilled with N₂. Then CO (10 atm) was introduced in. The resulting solution was stirred at 100° C. for overnight. The reaction mixture was filtered, and the filtrate was concentrated to dryness. The residue was purified by chromatography: Column, C18; mobile phase A: H₂O with 0.05% TFA, B: ACN; gradient 38% B to 58% B in 20 minutes; Detector: UV@210 nm. It gave methyl 6-(2,2-dimethyl-4-oxo-3,8,11-trioxa-5-azatridecan-13-oyl)picolinate (2.4 g, 63% yield) as a brown oil. MS (ES, m/z): 405 (M+Na⁺).

Step 4: To a solution of methyl 6-(2,2-dimethyl-4-oxo-3,8,11-trioxa-5-azatridecan-13-oyl)picolinate (2.30 g, 6.01 mmol) in MeOH (46 mL) under N₂ atmosphere at 0° C. was added NaBH₄ (0.23 g, 6.01 mmol). The resulting solution was stirred for 1 h at room temperature and quenched by the addition of 50 mL of saturated NH₄HCO₃ (aq.). The resulting solution was extracted with ethyl acetate (30 mL×2). The combined organic layers were washed with brine (60 mL), dried over Na₂SO₄ and concentrated. It gave 2.2 g of the crude product methyl 6-(13-hydroxy-2,2-dimethyl-4-oxo-3,8,11-trioxa-5-azatridecan-13-yl)picolinate as brown oil.

Step 5: To a solution of methyl 6-(13-hydroxy-2,2-dimethyl-4-oxo-3,8,11-trioxa-5-azatridecan-13-yl)picolinate (2.2 g, 5.72 mmol) in dichloromethane (22 mL) at 0° C. under N₂ atmosphere were added triethylamine (1.74 g, 17.16 mmol) and MsCl (0.79 g, 6.86 mmol). The resulting solution was stirred for 1 h at room temperature and quenched with H₂O (22 mL). The resulting mixture was extracted with dichloromethane (20 mL×2). The combined organic layers were washed with brine (40 mL), dried over Na₂SO₄ and concentrated. It gave 2.2 g of the crude product methyl 6-(2,2-dimethyl-13-((methylsulfonyl)oxy)-4-oxo-3,8,11-trioxa-5-azatridecan-13-yl)picolinate as brown oil.

Step 6: To a solution of methyl 6-(2,2-dimethyl-13-((methylsulfonyl)oxy)-4-oxo-3,8,11-trioxa-5-azatridecan-13-yl)picolinate (2.2 g, 4.75 mmol) in ACN (22 mL) under N₂ atmosphere was added NaI (0.78 g, 5.23 mmol). The resulting solution was stirred for 1 h at 80° C. The mixture was filtered and concentrated. The crude product was purified by chromatography: Column, C18; mobile phase A: H₂O, B: ACN; gradient 50% B to 80% B in 30 min; Detector: UV@210 nm. It gave methyl 6-(13-iodo-2,2-dimethyl-4-oxo-3,8,11-trioxa-5-azatridecan-13-yl)picolinate (1.2 g) as brown oil. MS (ES, m/z): 517 (M+Na⁺), 495 (M+H⁺).

Step 7: A solution of methyl 6-(13-iodo-2,2-dimethyl-4-oxo-3,8,11-trioxa-5-azatridecan-13-yl)picolinate (840 mg, 1.69 mmol) and methyl 6-((1,4,10,13-tetraoxa-7,16-diazacyclooctadecan-7-yl)methyl)picolinate (839 mg, 2.03 mmol) in ACN (16.8 mL) was stirred for overnight at 80° C. under nitrogen atmosphere. The cooled reaction mixture was filtered, and the filtrate was concentrated under reduced pressure. The crude product was purified by chromatography: Column, C18; mobile phase A: H₂O, B: ACN; gradient 40% B to 60% B in 20 min; Detector: UV@210 nm. It gave methyl 6-((16-(13-(6-(methoxycarbonyl)pyridin-2-yl)-2,2-dimethyl-4-oxo-3,8,11-trioxa-5-azatridecan-13-yl)-1,4,10,13-tetraoxa-7,16-diazacyclooctadecan-7-yl)methyl)picolinate (450 mg) as a brown oil. MS (ES, m/z): 700 (M+Na⁺), 678 (M+H⁺).

Step 8: To a solution of methyl 6-((16-(13-(6-(methoxycarbonyl)pyridin-2-yl)-2,2-dimethyl-4-oxo-3,8,11-trioxa-5-azatridecan-13-yl)-1,4,10,13-tetraoxa-7,16-diazacyclooctadecan-7-yl)methyl)picolinate (450 mg, 579 mmol) in dichloromethane (2.5 mL) at 0° C. was added HCl/dioxane (2.5 ml, 4 M). The resulting solution was stirred for 20 min at room temperature. The reaction was quenched by the addition of saturated Na₂CO₃ (aq.). The aqueous layer was extracted with DCM:IPA (5:1) (30 mL×2). The combined organic layers were dried over anhydrous Na₂SO₄ and concentrated under reduced pressure to afford the crude product methyl 6-(2-(2-(2-aminoethoxy)ethoxy)-1-(16-((6-(methoxycarbonyl)pyridin-2-yl)methyl)-1,4,10,13-tetraoxa-7,16-diazacyclooctadecan-7-yl)ethyl)picolinate (330 mg). The crude product was used directly in the next step.

Step 9: A solution of methyl 6-(2-(2-(2-aminoethoxy)ethoxy)-1-(16-((6-(methoxycarbonyl)pyridin-2-yl)methyl)-1,4,10,13-tetraoxa-7,16-diazacyclooctadecan-7-yl)ethyl)picolinate (300 mg, 0.44 mmol) and 1-(2-oxopyridine-1-carbothioyl) pyridin-2-one (113.08 mg, 0.48 mmol) in dichloromethane (3 mL) was stirred for 1 h at room temperature under nitrogen atmosphere. The resulting mixture was concentrated under reduced pressure to afford the crude product methyl 6-(2-(2-(2-isothiocyanatoethoxy)ethoxy)-1-(16-((6-(methoxycarbonyl)pyridin-2-yl)methyl)-1,4,10,13-tetraoxa-7,16-diazacyclooctadecan-7-yl)ethyl)picolinate (380 mg). The crude product was used directly in the next step.

Step 10: A solution of methyl 6-(2-(2-(2-isothiocyanatoethoxy)ethoxy)-1-(16-((6-(methoxycarbonyl)pyridin-2-yl)methyl)-1,4,10,13-tetraoxa-7,16-diazacyclooctadecan-7-yl)ethyl)picolinate (380 mg, 0.52 mmol) and HCl (1.9 mL, 6 M) in dichloromethane (1.9 mL) was stirred for 3 h at 50° C. under nitrogen atmosphere. The resulting mixture was concentrated under reduced pressure and was basified to pH 6-7 with saturated NaHCO₃(aq.). The residue was purified by chromatography: Column, C18; mobile phase A: H₂O with 0.05% TFA, B: ACN, gradient 20% B to 36% B in 20 min; Detector: UV@210 nm. Then, the product fractions were concentrated under vacuum to remove MeCN. The solution was purified again by chromatography: Column, C18; mobile phase A: H₂O, B: ACN, gradient 95% B to 100% B in 20 min. The solution was concentrated to remove most MeCN and the aqueous solution was lyophilized to give 6-((16-(1-(6-carboxypyridin-2-yl)-2-(2-(2-isothiocyanatoethoxy)ethoxy)ethyl)-1,4,10,13-tetraoxa-7,16-diazacyclooctadecan-7-yl)methyl)picolinic acid (130 mg) as brown solid. ¹H NMR (300 MHz, D₂O) 8.03-7.84 (m, 2H), 7.57 (dd, J=22.3, 7.4 Hz, 1H), 5.00 (s, OH), 4.59 (s, 1H), 4.20 (dd, J=23.4, 9.5 Hz, 1H), 3.82 (d, J=15.4 Hz, 4H), 3.70-3.58 (m, 6H), 3.58-3.49 (m, 6H). MS (ES, m/z): 692.3 (M+H⁺).

### Example 15

### 6,6′-(((S)-2-(((5-(((((1R,8S,9r)-Bicyclo[6.1.0]non-4-yn-9-yl)methoxy)carbonyl)amino)pentyl)thio)methyl)-1,4,10,13-tetraoxa-7,16-diazacyclooctadecane-7,16-diyl)bis(methylene))dipicolinic acid

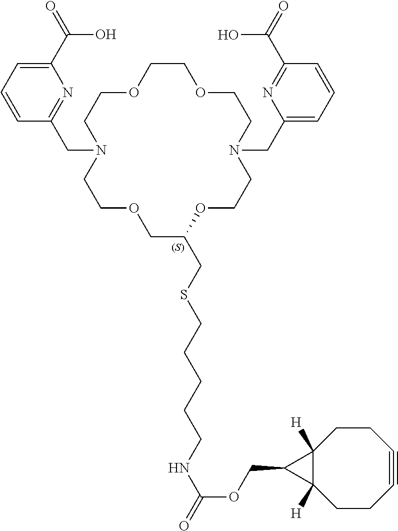

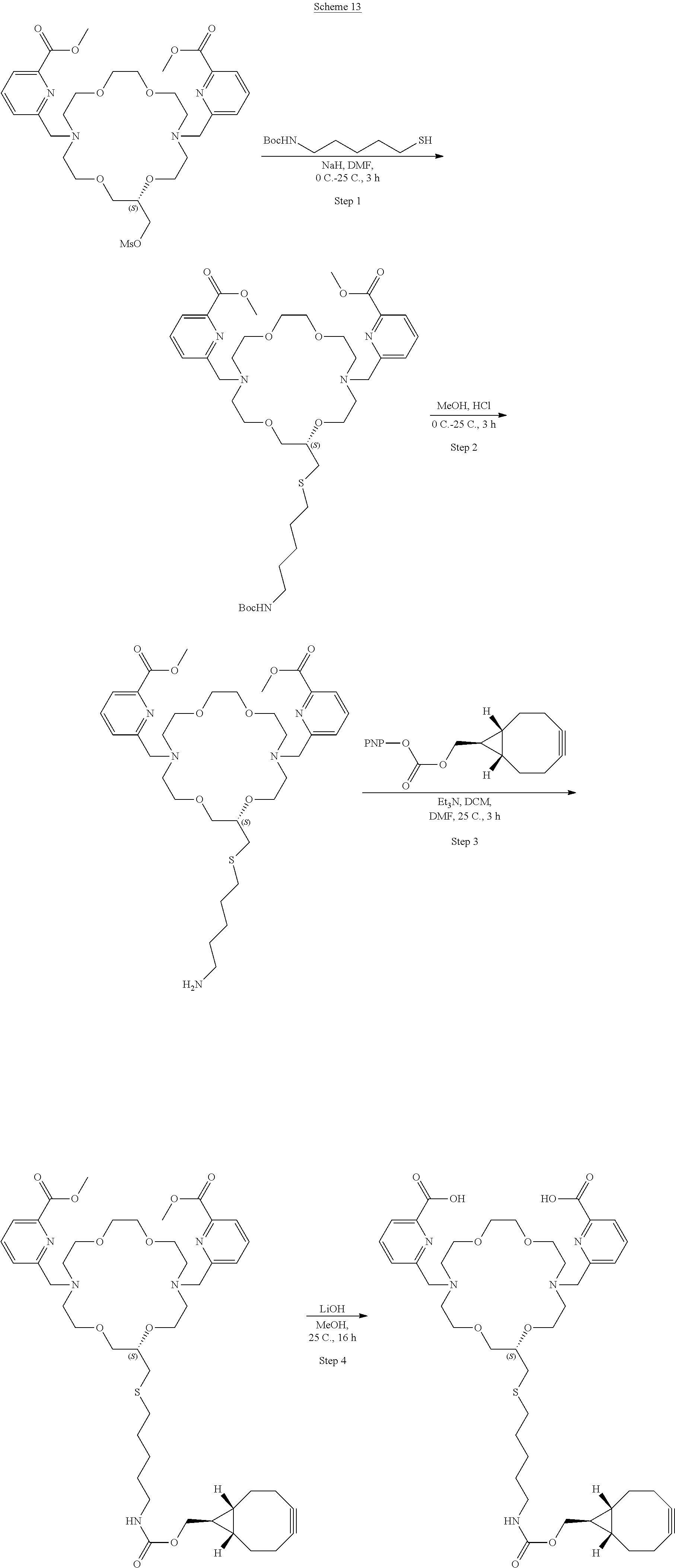

Step 1: A solution consisting of tert-butyl (5-mercaptopentyl)carbamate (0.30 g, 1.0 mmol) and DMF (3.0 mL) was added dropwise over 5 minutes to a 50 mL three-neck round-bottomed flask containing a suspension of sodium hydride (0.07 g, 60% in mineral oil, 2 mmol) in DMF (3.0 mL) at 0° C. and under a nitrogen atmosphere. Once addition was complete, the reaction mixture was brought to room temperature and stirring continued for 15 minutes. The reaction mixture was then re-cooled to 0° C. and treated dropwise over 10 minutes with a solution consisting of dimethyl 6,6′-((2-(((methylsulfonyl)oxy)methyl)-1,4,10,13-tetraoxa-7,16-diazacyclooctadecane-7,16-diyl)bis(methylene))(S)-dipicolinate (0.6 g, 0.9 mmol) and DMF (3.0 mL). Once addition was complete, the reaction mixture was slowly warmed to room temperature and stirring continued for 1.5 h. The reaction mixture was then carefully treated with sat. aqueous NH₄Cl (1.0 mL) and concentrated to dryness to give an oil. The oil was subjected to preparative HPLC (Column: XBRIDGE C18 19×150 mm, 5.0 μm; Mobile phase: 0.1% TFA in water/acetonitrile; Flow Rate: 15.0 mL/min) to yield dimethyl 6,6′-((2-(((5-((tert-butoxycarbonyl)amino)pentyl)thio)methyl)-1,4,10,13-tetraoxa-7,16-diazacyclooctadecane-7,16-diyl)bis(methylene))(S)-dipicolinate (0.25 g).

Step 2: A stir bar, dimethyl 6,6′-((2-(((5-((tert-butoxycarbonyl)amino)pentyl)thio)methyl)-1,4,10,13-tetraoxa-7,16-diazacyclooctadecane-7,16-diyl)bis(methylene))(S)-dipicolinate (0.25 g, 0.32 mmol), MeOH (1.0 mL), and HCl in methanol (4 M, 1.5 mL, 6.3 mmol) were added to a 25 mL round-bottomed flask at 0° C., which was subsequently brought to room temperature and the mixture stirred for 3 h. The volatiles were then removed in vacuo to yield dimethyl 6,6′-((2-(((5-aminopentyl)thio)methyl)-1,4,10,13-tetraoxa-7,16-diazacyclooctadecane-7,16-diyl)bis(methylene))(S)-dipicolinate (0.21 g), which was used without purification.

Step 3: A stir bar, dimethyl 6,6′-((2-(((5-aminopentyl)thio)methyl)-1,4,10,13-tetraoxa-7,16-diazacyclooctadecane-7,16-diyl)bis(methylene))(S)-dipicolinate (0.15 g, 0.22 mmol), ((1R,8S,9r)-bicyclo[6.1.0]non-4-yn-9-yl)methyl 4-nitrophenyl carbonate (68 mg, 0.22 mmol), triethylamine (66 mg, 0.65 mmol), and a mixture of DCM (2 mL) and DMF (0.1 mL) were added to a 25 mL three-neck round-bottomed flask at 0° C. under a nitrogen atmosphere. The resultant mixture was gradually warmed to room temperature and stirred overnight. The mixture was then concentrated to dryness to give dimethyl 6,6′-(((S)-2-(((5-(((((1R,8S,9r)-bicyclo[6.1.0]non-4-yn-9-yl)methoxy)carbonyl)amino)pentyl)thio)methyl)-1,4,10,13-tetraoxa-7,16-diazacyclooctadecane-7,16-diyl)bis(methylene))dipicolinate (0.1 g), which was used without purification.

Step 4: A stir bar, dimethyl 6,6′-(((S)-2-(((5-(((((1R,8S,9r)-bicyclo[6.1.0]non-4-yn-9-yl)methoxy)carbonyl)amino)pentyl)thio)methyl)-1,4,10,13-tetraoxa-7,16-diazacyclooctadecane-7,16-diyl)bis(methylene))dipicolinate (0.10 g, 0.12 mmol), aqueous LiOH (3.5 mL, 0.1 N, 0.35 mmol), and MeOH (0.5 mL) were added to a 8 mL reaction vial and the resultant mixture stirred overnight at room temperature. The reaction mixture was then treated with acetic acid until pH-6.5, and subsequently concentrated to dryness in vacuo at room temperature to yield an oil, which was purified via preparative HPLC (Column: XBRIDGE C18 19×150 mm, 5.0 μm; Mobile phase: 0.1% Formic acid in H₂O/ACN; Flow Rate: 15.0 mL/min) to yield 6,6′-(((S)-2-(((5-(((((1R,8S,9r)-bicyclo[6.1.0]non-4-yn-9-yl)methoxy)carbonyl)amino)pentyl)thio)methyl)-1,4,10,13-tetraoxa-7,16-diazacyclooctadecane-7,16-diyl)bis(methylene))dipicolinic acid (42 mg).

### Example 16

### N-acyl-DBCO tagged 6,6′-((2-(((5-Aminopentyl)thio)methyl)-1,4,10,13-tetraoxa-7,16-diazacyclooctadecane-7,16-diyl)bis(methylene))(S)-dipicolinic acid

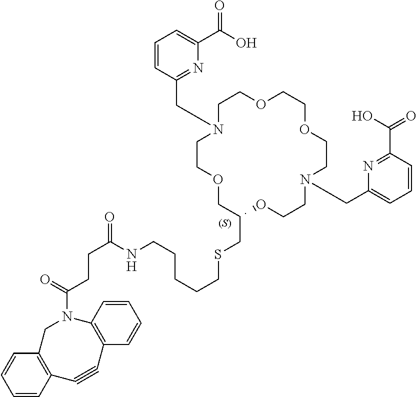

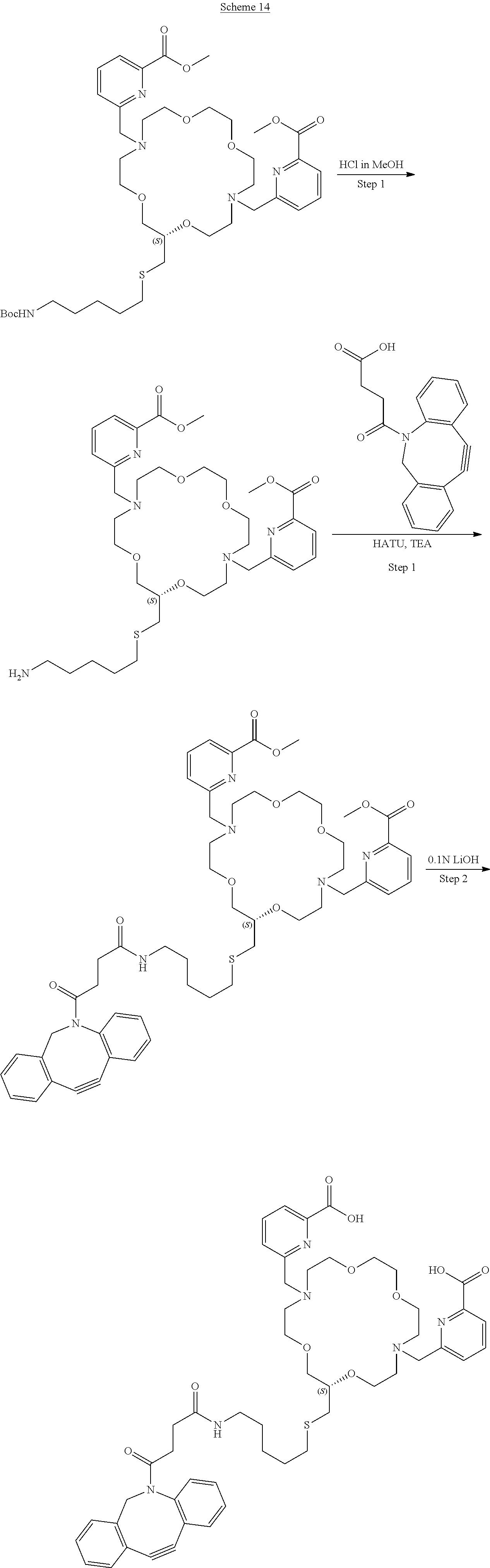

Step 1: A stir bar, dimethyl 6,6′-((2-(((5-((tert-butoxycarbonyl)amino)pentyl)thio)methyl)-1,4,10,13-tetraoxa-7,16-diazacyclooctadecane-7,16-diyl)bis(methylene))(S)-dipicolinate (0.12 g, 0.15 mmol), MeOH (0.5 mL), and HCl in methanol (4 M, 0.75 mL, 3.0 mmol) were added to a 25 mL round-bottomed flask at 0° C. and then brought to room temperature and stirred for 2 h. The volatiles were removed in vacuo to yield dimethyl 6,6′-((2-(((5-aminopentyl)thio)methyl)-1,4,10,13-tetraoxa-7,16-diazacyclooctadecane-7,16-diyl)bis(methylene))(S)-dipicolinate (70 mg), which was used without purification.

Step 2: A stir bar, dimethyl 6,6′-((2-(((5-aminopentyl)thio)methyl)-1,4,10,13-tetraoxa-7,16-diazacyclooctadecane-7,16-diyl)bis(methylene))(S)-dipicolinate (50 mg, 0.070 mmol), 11,12-Didehydro-γ-oxodibenz[b,f]azocine-5(6H)-butanoic acid (20 mg, 0.070 mmol), triethylamine (21 mg, 0.21 mmol), HATU (38 mg, 0.10 mmol), and DCM (0.5 mL) were added to a 25 mL three-neck round-bottomed flask at 0° C. under a nitrogen atmosphere, and subsequently brought to room temperature and stirred overnight. The reaction mixture was treated with water (10 mL) and extracted with dichloromethane (10 mL×3), and the combined extracts washed with 10% aqueous NaHCO₃ (10 mL) and brine (10 mL), dried over anhydrous Na₂SO₄, filtered, and concentrated to dryness to yield an oil. The oil was purified via silica gel chromatography (0-10% MeOH/DCM) to yield N-acyl-DBCO tagged dimethyl 6,6′-((2-(((5-aminopentyl)thio)methyl)-1,4,10,13-tetraoxa-7,16-diazacyclooctadecane-7,16-diyl)bis(methylene))(S)-dipicolinate (16 mg).

Step 3: A stir bar, N-acyl-DBCO tagged dimethyl 6,6′-((2-(((5-aminopentyl)thio)methyl)-1,4,10,13-tetraoxa-7,16-diazacyclooctadecane-7,16-diyl)bis(methylene))(S)-dipicolinate (16 mg, 0.016 mmol), aqueous LiOH (0.49 mL, 0.1 N, 0.049 mmol), and MeOH (0.25 mL) were added to an 8 mL reaction vial and the mixture stirred at room temperature overnight. The reaction mixture was then treated with acetic acid until pH-6.5, and concentrated to dryness in vacuo at room temperature to yield a concentrate, which was purified via preparative HPLC (Column: XBRIDGE C18 19×150 mm 5.0 μm; Mobile phase: 10 mM Ammonium Acetate in water/ACN; Flow Rate: 15.0 mL/min) to yield N-acyl-DBCO tagged 6,6′-((2-(((5-aminopentyl)thio)methyl)-1,4,10,13-tetraoxa-7,16-diazacyclooctadecane-7,16-diyl)bis(methylene))(S)-dipicolinic acid (5 mg) as an off-white solid. LC-MS APCI: Calculated for C₅₁H₆₂N₆O₁₀S; 950.42; Observed m/z [M+H]⁺ 951.4. ¹H NMR (400 MHz, D₂O): δ 7.81-7.75 (m, 4H), 7.52-7.17 (m, 10H), 4.97-4.90 (m, 1H), 4.80 (s, 4H), 4.14 (s, 3H), 3.77-3.46 (m, 16H), 3.10 (s, 7H), 2.83-2.80 (m, 2H), 2.55-2.53 (m, 2H), 2.42-2.38 (m, 3H), 2.11-2.08 (m, 3H), 1.39-1.35 (m, 2H), 1.20-1.10 (m, 4H).

### Example 17

### N-acyl-DBCO tagged 6-((4-((6-Aminoethyl)carbamoyl)phenyl)16-((6-carboxypyridin-2-yl)-1,4,10,13-tetraoxa-7,16-diazacyclooctadecan-7-yl)methyl)picolinic acid (TOPA-[C7]-benzimido-DBCO)

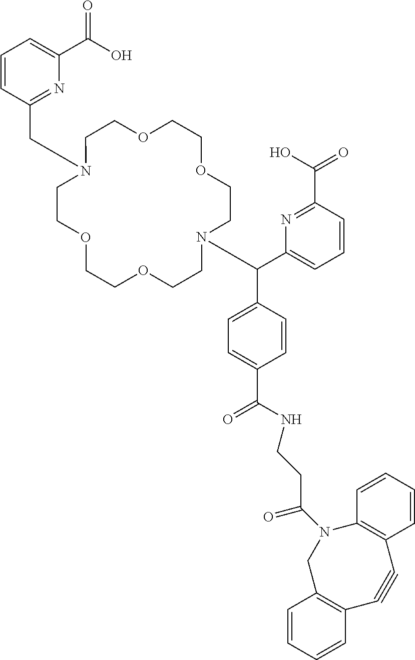 [SMILES: O=C(NCCC(=O)N1Cc2ccccc2C#Cc2ccccc21)c1ccc(C(c2cccc(C(=O)O)n2)N2CCOCCOCCN(Cc3cccc(C(=O)O)n3)CCOCCOCC2)cc1]

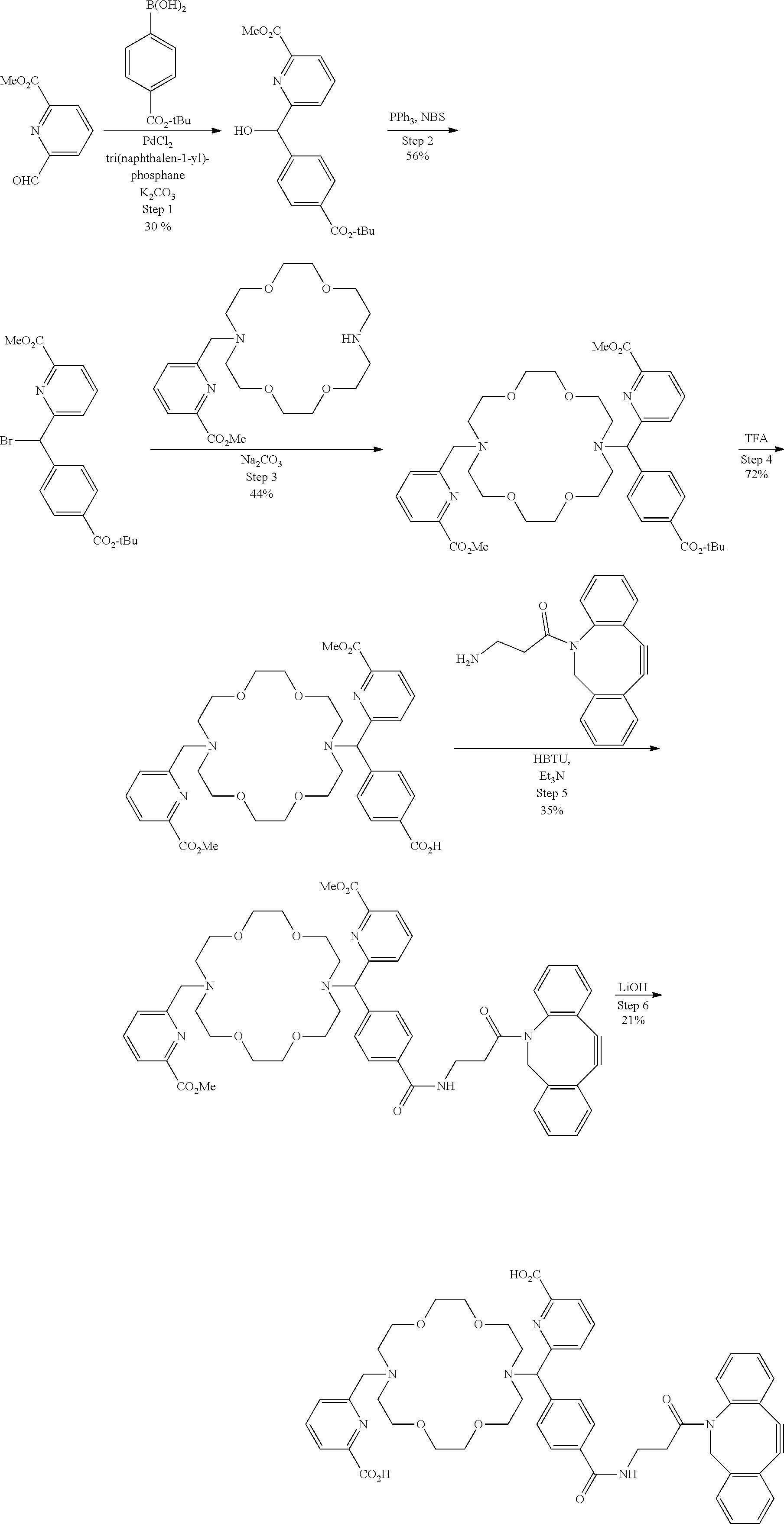

Step 1: To a mixture of methyl 6-formylpicolinate (4.00 g, 24.2 mmol), (4-(tert-butoxycarbonyl)phenyl)boronic acid (10.7 g, 48.5 mmol), PdCl₂ (0.21 g, 1.2 mmol), tri(naphthalen-1-yl)phosphine (0.50 g, 1.2 mmol) and potassium carbonate (10.0 g, 72.7 mmol) under nitrogen at −78° C. in a 500 mL three neck round bottom flask was added tetrahydrofuran (100 mL) in one portion. The mixture was purged with nitrogen and stirred at r.t. for 30 min, then heated at 65° C. for 24 h. The reaction mixture was cooled r.t. and filtered through a pad of Celite® and the filtrate was concentrated to dryness. The crude product was subjected to silica gel chromatography (0-50% EtOAc/petether) to afford methyl 6-((4-(tert-butoxycarbonyl)phenyl)(hydroxy)methyl)picolinate as a yellow oil (2.5 g, 30%).

Step 2: A stir bar, methyl 6-((4-(tert-butoxycarbonyl)phenyl)(hydroxy)methyl)picolinate (2.50 g, 7.30 mmol), PPh₃ (3.43 g, 13.1 mmol), N-bromosuccinimide (2.13 g, 12.0 mmol) and DCM (30 mL) were taken in a 250 mL three neck round bottom flask under nitrogen atmosphere at r.t. and stirred for 1 h. The reaction solution was loaded onto a silica gel column and purified using 0-30% ethyl acetate in petroleum ether to get compound methyl 6-(bromo(4-(tert-butoxycarbonyl)phenyl)methyl)picolinate (1.65 g, 56%) as a yellow oil.

Step 3: A stir bar, methyl 6-((1,4,10,13-tetraoxa-7,16-diazacyclooctadecan-7-yl)methyl)picolinate (1.52 g, 3.69 mmol), 6-(bromo(4-(tert-butoxycarbonyl)phenyl)methyl)picolinate (1.50 g, 3.69 mmol), Na₂CO₃ (1.17 g, 11.1 mmol), and acetonitrile (30 mL) were added to a 250 mL three neck round-bottomed flask, and the resultant heterogeneous mixture was heated at 90° C. for 16 h under nitrogen atmosphere. Subsequently reaction mass was cooled to r.t., filtered through a pad of Celite®, and concentrated to dryness in vacuo to give the crude product. The crude product was subjected to silica gel chromatography (0-10% MeOH/DCM) to afford methyl 6-((4-(tert-butoxycarbonyl)phenyl)(16-((6-(methoxycarbonyl)pyridin-2-yl)methyl)-1,4,10,13-tetraoxa-7,16-diazacyclooctadecan-7-yl)methyl)picolinate as a brown oil (1.2 g, 44%).

Step 4: A stir bar, methyl 6-((4-(tert-butoxycarbonyl)phenyl)(16-((6-(methoxycarbonyl)pyridin-2-yl)methyl)-1,4,10,13-tetraoxa-7,16-diazacyclooctadecan-7-yl)methyl)picolinate (1.2 g, 1.6 mmol), TFA (0.62 mL, 8.1 mmol) and DCM (20 mL) were added to a 100 mL three neck round bottom flask at r.t. and stirred for 1 h. Reaction mixture was concentrated to dryness and the resultant crude product was subjected to preparative HPLC (Column: XBRIDGE C18 (19×150 mm) 5.0 μm; Mobile phase: 0.1% TFA in water/ACN; Flow Rate: 15.0 mL/min) to give 4-((6-(methoxycarbonyl)pyridin-2-yl)(16-((6-(methoxycarbonyl)pyridin-2-yl)methyl)-1,4,10,13-tetraoxa-7,16-diazacyclooctadecan-7-yl)methyl)benzoic acid (0.8 g, 72%) as brown oil. LC-MS APCI: Calculated for C₃₅H₄₄N₄O₁₀ 680.31; Observed m/z [M+H]⁺ 681.5. Purity by LC-MS: 99.87%. Purity by HPLC: 97.14% (97.01% at 210 nm, 97.20% at 254 nm and 97.21% at 280 nm; Column: Atlantis dC18 (250×4.6 mm), 5 μm; Mobile phase A: 0.1% TFA in water, Mobile phase B: acetonitrile; Flow rate: 1.0 mL/min.%. ¹H NMR (400 MHz, DMSO-d₆): δ 8.12-8.07 (m, 4H), 8.00-7.98 (m, 2H), 7.75-7.73 (m, 4H), 6.10 (s, 1H), 4.67 (s, 2H), 3.96 (s, 3H), 3.91 (s, 3H), 3.82 (s, 8H), 3.56 (s, 8H), 3.52 (s, 8H).

Step 5: A stir bar, 4-((6-(methoxycarbonyl)pyridin-2-yl)(16-((6-(methoxycarbonyl)pyridin-2-yl)methyl)-1,4,10,13-tetraoxa-7,16-diazacyclooctadecan-7-yl)methyl)benzoic acid (0.25 g, 0.37 mmol), DBCO (0.10 g, 0.37 mmol), triethylamine (0.16 mL, 1.1 mmol), HBTU (0.21 g, 0.55 mmol) and DCM (10 mL) were added to a 25 mL three neck round-bottom flask at 0° C. under nitrogen atmosphere at r.t. and stirred for 16 h. The reaction was quenched with water (20 mL) and it was extracted with DCM (3×20 mL). The combined extracts were washed with 10% aqueous NaHCO₃ solution (20 mL), brine (20 mL), dried over anhydrous Na₂SO₄, filtered, and concentrated to dryness to afford the crude product as an oil. The crude product was subjected to silica gel chromatography (0-10% MeOH/DCM) to give TOPA dimethyl ester-[C7]-phenyl-DBCO (0.12 g, 35%) as a colorless gummy oil.

Step 6: A stir bar, TOPA dimethyl ester-[C7]-phenyl-DBCO (0.1 g, 0.1 mmol), aqueous LiOH·H₂O (3 mL, 0.1 N, 0.3 mmol) and THF/MeOH/H₂O (4:1:1 v/v/v, 2 mL) were added to a 8 mL reaction vial at r.t. and it was allowed to stir for 2 h. The reaction mixture was neutralized with aqueous HCl (1N) to P^{H}˜6.5. The reaction mixture was concentrated to dryness in vacuo at room temperature, and the resultant crude product was subjected to preparative HPLC (Column: XBRIDGE C18 (19×150 mm) 5.0 μm; Mobile phase: 10 Mm Ammonium Acetate in water/ACN; Flow Rate: 15.0 mL/min) to give TOPA-[C7]-phenyl-DBCO (20 mg, 21%) as an off-white solid. LC-MS APCI: Calculated for C₅₁H₅₄N₆O₁₀ 910.39; Observed m/z [M−H]⁺ 909.3. Purity by LC-MS: 92.47%. Purity by HPLC: 90.68% (88.04% at 210 nm, 90.43% at 254 nm and 93.56% at 280 nm; Column: XBRIDGE C8 (50×4.6 mm), 3.5 μm; Mobile phase A: 10 mM Ammonium bicarbonate in water, Mobile phase B: acetonitrile; Flow rate: 1.0 mL/min. ¹H NMR (400 MHz, DMSO-d6): δ 7.84-7.82 (m, 4H), 7.60-7.29 (m, 12H), 7.13-7.10 (m, 2H), 5.12-5.02 (m, 2H), 3.97 (s, 2H), 3.59-3.44 (m, 20H), 2.85 (s, 4H), 2.73-2.68 (m, 6H).

### Example 18

### TOPA-[C7]-benzyimido-DBCO-triazole-PSMB-127 Antibody Conjugate

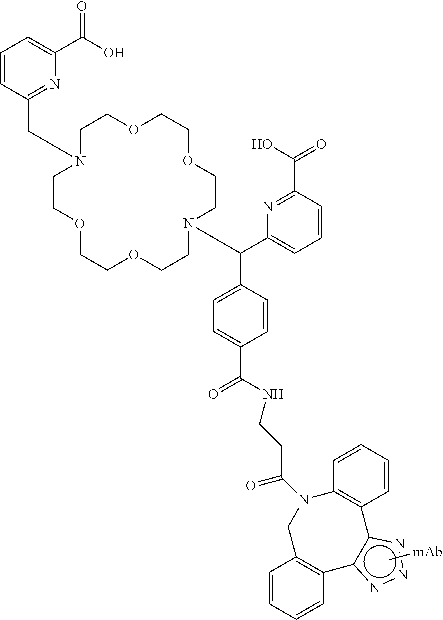

Step 1. Azide modification of mAb and Click reaction: PSMB127 was site-selectively modified with 100× molar excess of 3-azido propylamine and microbial transglutaminase (MTG; Activa TI) at 37° C. The addition of two azides on the heavy chains of the mAb was monitored by intact mass ESI-TOF LC-MS on an Agilent G224 instrument. Excess 3-azido propylamine and MTG was removed and azide modified mAb (azido-mAb) was purified using a 1 mL GE Healthcare MabSelect column. Azido-mAb is eluted from the resin using 100 mM sodium citrate pH 3.0 and subsequently exchanged into 20 mM Hepes, 100 mM NaCl pH 7.5 using 7K Zeba® desalting columns. 10× molar excess of TOPA-[C7]-phenyl-DBCO was reacted with site specific azide-PSMB127 (DOL=2) at 37° C. for 1 hour without shaking. Completion of the DBCO-azide click reaction was monitored by intact mass spectrometry. Excess free chelator was removed by desalting the conjugate over a Zeba @7K desalting column into 20 mM Hepes, 100 mM NaCl pH 7.5 followed by three sequential 15× dilution and concentration steps in 20 mM Hepes, 100 mM NaCl pH 7.5 using a 30K MWCO Amicon concentrator device by spinning at 3800×g. This provided the final site specific TOPA-[C7]-phenyl-DBCO-PSMB127 conjugate with CAR=2. The final conjugate was confirmed to be monomeric by analytical size exclusion chromatography on a Tosoh TSKgel G3000SWxl 7.8 mm×30 cm, 5 u column; column temperature: room temperature; the column was eluted with DPBS buffer (1×, without calcium and magnesium); flow rate: 0.7 mL/min; 18 min run; injection volume: 18 L.

Step 2. Chelation: Stock solutions of the following metal salts were prepared in pure water:

| Salt | Catalog # | Concentration |
|---|---|---|
| Cerium (III) Chloride | Sigma Cat # 429406 | 10 mM |
| Neodymium (III) chloride | Sigma Cat # 289183 | 10 mM |
| hexahydrate | | |
| Terbium (III) chloride | Sigma Cat # 451304 | 10 mM |
| Lutetium (III) chloride | Sigma Cat # 450960 | 10 mM |
| Thulium (III) chloride | Sigma Cat # 451304 | 10 mM |
| Yttrium (III) chloride | Sigma Cat # 451363 | 10 mM |
| Holmium (III) chloride | Sigma Cat # 450901 | 10 mM |

Metal solutions were added to the TOPA-[C7]-phenyl-DBCO-PSMB127 in 5× molar excess (6.8 uM antibody, 34 uM metal ion) in 10 mM sodium acetate buffer pH 5.2 and incubated for 2 hours at 37 C. Excess metal was removed by desalting with a Zeba® column (ThermoFisher®) followed by two cycles of 10× dilution and concentration in a 50K MWCO Amicon concentrator (EMID Millipore @). Chelation was assessed by intact and reduced mass LC-MS.

Step 3. Stability Determination: To determine stability of the chelate, DTPA challenge was performed. 50 μL of the sample (6.3 uM antibody) was combined with 50 μL of 10 mM DTPA pH 6.5 and incubated at 37 C overnight. Chelation was assessed by intact and reduced mass LC-MS. LC-MS was performed on an Agilent 1260 HPLC system connected to an Agilent G6224 MS-TOF Mass Spectrometer. LC was run on an Agilent RP-mAb C4 column (2.1×50 mm, 3.5 micron) at a flow rate of 1 mL/min with the mobile phase 0.1% formic acid in water (A) and 0.1% formic acid in acetonitrile (Sigma-Aldrich Cat #34688) (B) and a gradient of 20% B (0-2 min), 20-60% B (2-3 min), 60-80% B (3-5.5 min). The instrument was operated in positive electro-spray ionization mode and scanned from m/z 600 to 6000. Mass to charge spectrum was deconvoluted using the Maximum Entropy algorithm, and relative amounts of the relevant species were estimates by peak heights of the deconvoluted masses. Instrument settings included: capillary voltage 3500V; fragmentor 175V; skimmer 65V; gas temperature 325 C; drying gas flow 5.0 L/min; nebulizer pressure 30 psig; acquisition mode range 100-7000 with 0.42 scan rate.

Changes in MW relative to the TOPA-[C7]-phenyl-DBCO-PSMB127 were observed for the cerium and neodymium samples. The intact mass of the conjugate incubated with cerium showed an increase in MW of 139 (20% by peak area) or 276 (77%) Da corresponding to the addition of 1 or 2 cerium ions. After DTPA challenge, the masses remained similar with similar abundance (30 and 67% for the +138 and +274 species).

### Example 19

### 6-((16-((6-carboxypyridin-2-yl)(4-isothiocyanatophenyl)methyl)-1,4,10,13-tetraoxa-7,16-diazacyclooctadecan-7-yl)methyl)picolinic acid (H₂bp18c6-benzyl-phenyl) (TOPA-[C7]-phenylisothiocyanate and Sodium Salt Forms

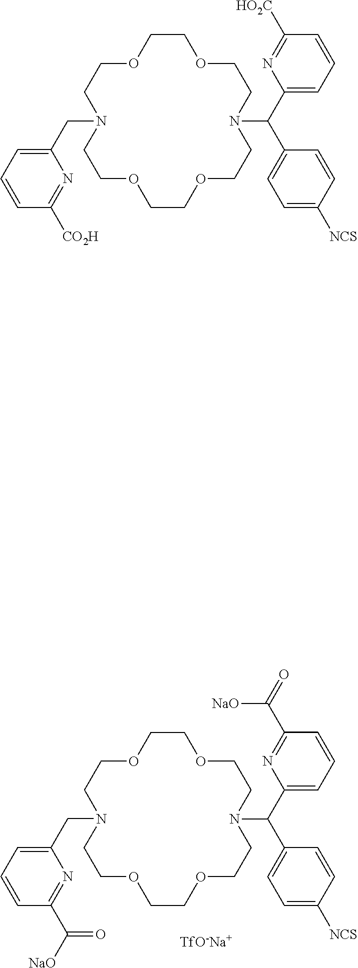

Compound 2 was prepared in an analogous manner to existing literature methods see. J. Org. Chem; 1987, 52, 5172.

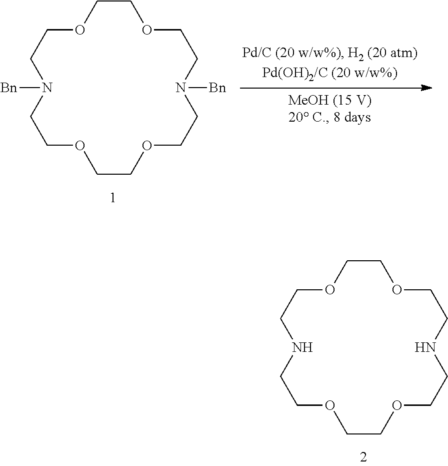 [SMILES: c1ccc(CN2CCOCCOCCN(Cc3ccccc3)CCOCCOCC2)cc1 ; C1COCCOCCNCCOCCOCCN1]

Compound 3 was prepared in an analogous manner to existing literature methods see. Chemistry—a European Journal; 2015, 21, 10179.

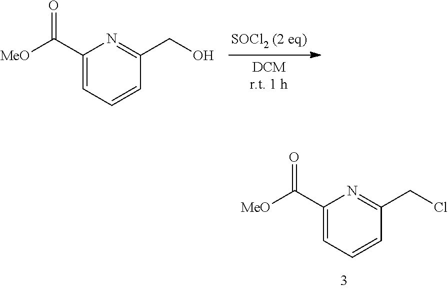 [SMILES: COC(=O)c1cccc(CO)n1 ; COC(=O)c1cccc(CCl)n1]

### Preparation of Compound 4

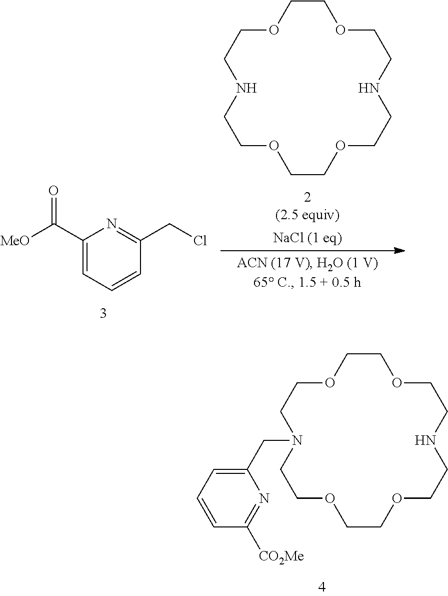 [SMILES: C1COCCOCCNCCOCCOCCN1 ; COC(=O)c1cccc(CCl)n1 ; COC(=O)c1cccc(CN2CCOCCOCCNCCOCCOCC2)n1]

1,4,10,13-tetraoxa-7,16-diazacyclooctadecane (494 g, 1.88 mol, 2.5 equiv.), NaCl (44.1 g, 0.75 mol, 1.0 equiv.), H₂O (140 mL, 1 volume with respect to compound 3) and acetonitrile (2.1 L, 15 volumes) were charged to a 10 L reactor under N₂ atmosphere at 15-20° C. the heated to 65° C. To the resulting mixture was added a solution of compound 3 (140 g, 0.75 mol) in acetonitrile (280 ml, 2 volumes) dropwise over 1 hour 65° C. The solution was aged at 65° C. for 0.5 hours. LCMS analysis of the mixture showed the reaction was completed. The mixture was cooled to room temperature and concentrated under vacuum. Acetone (700 ml, 5 volumes) was added to the mixture and the suspension was stirred for an additional 1 hour. The mixture was filtered (the filtered solid was unreacted compound 2). The filtrate was concentrated under vacuum, then dissolved in DCM (1.4 L, 10 volumes). The organic phase was washed with water (3×750 mL) and the organic phase was dried over Na₂SO₄ then concentrated under vacuum to yield compound 4, 212 g (63% yield, assay: 85% w/w). LCMS: (ES, m/z): 412.15 [M+H]⁺ ¹H-NMR (300 MHz, DMSO-d₆, ppm): δ 7.98-7.87 (m, 2H), 7.81 (dd, J=6.4, 2.6 Hz, 1H), 3.87 (s, 3H), 3.81 (s, 2H), 3.61-3.38 (m, 16H), 2.77 (dt, J=19.0, 5.2 Hz, 8H).

### Preparation of Compound 7

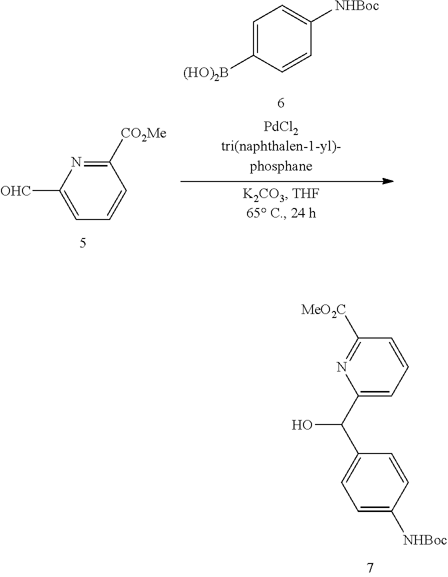 [SMILES: CC(C)(C)OC(=O)Nc1ccc(B(O)O)cc1 ; COC(=O)c1cccc(C=O)n1 ; COC(=O)c1cccc(C(O)c2ccc(NC(=O)OC(C)(C)C)cc2)n1]

Methyl 6-formylpicolinate 5 (250 g, 1.0 equiv.), (4-((tert-butoxycarbonyl)amino)phenyl)boronic acid 6 (538 g, 1.5 equiv.) and degassed THF (6.5 L, 26 volumes with respect to 5) were charged into a 10 L reactor under N₂ atmosphere at 15-20° C. This was followed by the addition of PdCl₂ (14.0 g, 0.05 equiv.), tri(naphthalen-1-yl)-phosphane (31 g, 0.05 equiv.) and K₂CO₃ (650 g, 3.1 equiv.). The resulting solution was stirred at 20° C. for 0.5 hours. The Mixture was then heated to 65° C. and aged for 17 hours. Analysis by LCMS showed this reaction was complete. The resulting solution was cooled at room temperature and was diluted with ice water (2.5 L, 10 volumes) and ethyl acetate (5 L, 20 volumes). The mixture was stirred then filtered through a celite pad. The solution was allowed to separate, and the aqueous lower layer was discarded. The organic phase was washed with the water (2×1.5 L, 12 volumes). The layers were separated, and the organic layer was dried over Na₂SO₄ and concentrated under vacuum. The resulting residue was treated with heptane (1.25 L, 5 Volumes) and the resulting suspension was stirred for 0.5 hours. The mixture was filtered, and the filter cake was washed with n-heptane (500 ml, 2 volumes) to yield 530 g (98% yield, LCAP purity: 90%) of desired product 7 as yellow solid, which was used directly in the next step without further purification. LCMS: (ES, m/z): 381.10 [M+Na]⁺ ¹H-NMR (300 MHz, DMSO-d₆, ppm): δ 9.27 (s, 1H), 8.03-7.85 (m, 2H), 7.79 (dd, J=7.7, 1.4 Hz, 1H), 7.39 (d, J=8.4 Hz, 2H), 7.26 (d, J=8.4 Hz, 2H), 6.13 (d, J=4.0 Hz, 1H), 5.72 (d, J=3.9 Hz, 1H), 3.87 (s, 3H), 1.46 (s, 9H).

### Preparation of Compound 8

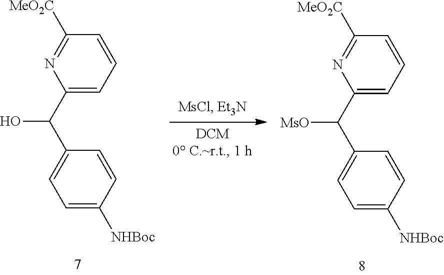 [SMILES: COC(=O)c1cccc(C(O)c2ccc(NC(=O)OC(C)(C)C)cc2)n1 ; COC(=O)c1cccc(C(OS(C)(=O)=O)c2ccc(NC(=O)OC(C)(C)C)cc2)n1]

Methyl 6-((4-((tert-butoxycarbonyl)amino)phenyl)(hydroxy)methyl)picolinate 7 (310 g, 1.0 equiv.), triethylamine (219 g, 2.5 equiv.) and DCM (6.2 L, 20 volumes with respect to 7) were charged into a 10 L reactor under nitrogen atmosphere at 15-20° C. and the solution was cooled to 0° C. Methanesulfonyl chloride (99.2 g, 1.0 equiv.) was added dropwise over 30 min maintaining the temperature at 0° C. The cooling bath was removed, and the temperature was allowed to reach ambient temperature and was then aged for 1 hour at this temperature. The solution was concentrated under vacuum at 10-15° C. and the residue was then dissolved in acetonitrile (438 ml, 2 volumes). The resulting solution was concentrated under vacuum to yield 518 g (crude) of desired product 8. This crude product was used for the next step directly without further purification.

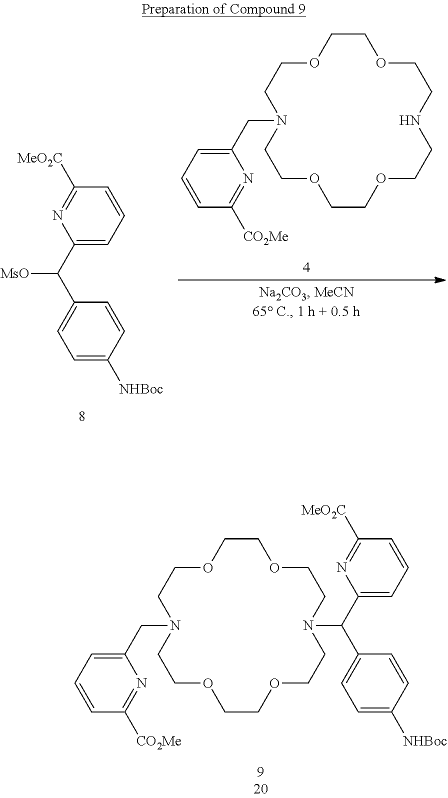 [SMILES: COC(=O)c1cccc(C(OS(C)(=O)=O)c2ccc(NC(=O)OC(C)(C)C)cc2)n1 ; COC(=O)c1cccc(CN2CCOCCOCCNCCOCCOCC2)n1 ; COC(=O)c1cccc(CN2CCOCCOCCN(C(c3ccc(NC(=O)OC(C)(C)C)cc3)c3cccc(C(=O)OC)n3)CCOCCOCC2)n1]

Methyl 6-((4-((tert-butoxycarbonyl)amino)phenyl)-((methylsulfonyl)oxy)methyl)picolinate 8 (212 g, 1.0 equiv. 85% purity by Q-NMR), Na₂CO₃ (137.6 g, 3.0 equiv.) and acetonitrile (3.56 L, 20 volumes with respect to 8) were charged into a 10 L reactor under a nitrogen atmosphere at room temperature then the mixture was heated to 65° C. and aged for 1 hour. A solution of methyl 6-((1,4,10,13-tetraoxa-7,16-diazacyclooctadecan-7-yl)methyl)picolinate 4 (377.8 g, 2.0 equiv.) in acetonitrile (3 L, 10 volumes) was added dropwise over 0.5 hours at 65° C. The mixture was aged at this temperature until HPLC analysis showed this reaction was completed. The resulting solution was cooled at room temperature then filtered, and the filter cake was washed by MeOH (2×1 volume). The filtrate was concentrated under vacuum and the resulting residue was dissolved in EA (700 mL), then silica gel (800 g, type: ZCX-2, 100-200 mesh, 2.11 w/w) was added. The mixture was concentrated under vacuum whilst maintaining the temperature below 35° C. Silica gel (9.6 kg, type: ZCX-2, 100-200 mesh, 26.3 w/w) was charged to the column, followed by the prepared dry silica gel containing adsorbed crude 9. The column was eluted with ethyl acetate:petroleum ether:dichloromethane (3:3:1)/methanol:dichloromethane (1:1) (gradient from 100:0 to 90:10 with sample collection every 4 L+0.5 L). The fractions were analyzed by TLC (ethyl acetate:petroleum ether:dichloromethane:methanol=4:4:1:1). The product bearing fractions were combined and concentrated to yield 260 g of compound 9 as yellow solid (HPLC: 94%, QNMR: 92%). An additional 70 g of compound 9 was afforded as yellow oil (HPLC: 75%, QNMR: 60%). LCMS (ES, m/z): 752.30 [M+H]⁺ Observed m/z ¹H-NMR (400 MHz, CDCl₃, ppm): δ 7.53-7.32 (m, 3H), 7.28-7.18 (m, 3H), 6.86 (d, J=8.4 Hz, 2H), 6.76 (d, J=8.4 Hz, 2H), 6.09 (s, 1H), 4.63 (s, 1H), 3.48 (s, 3H), 3.44 (bs, 5H), 3.17-2.92 (m, 16H), 2.38 (dq, J=25.0, 7.2, 6.8 Hz, 8H), 0.97 (s, 9H).

### Preparation of Compound 10

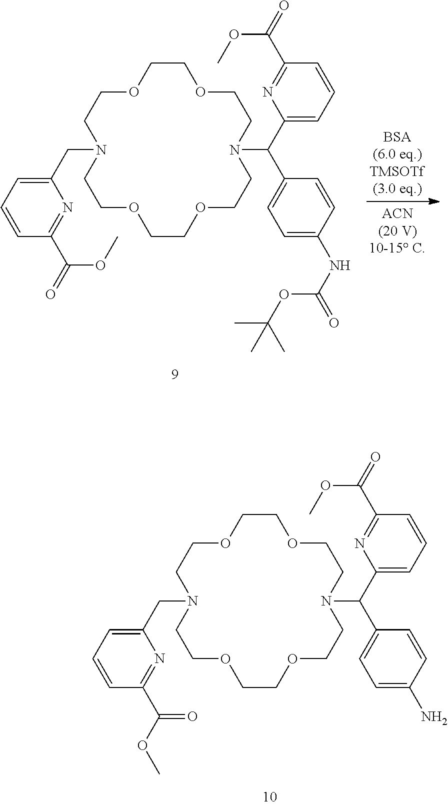 [SMILES: COC(=O)c1cccc(CN2CCOCCOCCN(C(c3ccc(NC(=O)OC(C)(C)C)cc3)c3cccc(C(=O)OC)n3)CCOCCOCC2)n1 ; COC(=O)c1cccc(CN2CCOCCOCCN(C(c3ccc(N)cc3)c3cccc(C(=O)OC)n3)CCOCCOCC2)n1]

Compound 9 (260 g, QNMR: 92%, 1.0 equiv.), N,O-bis(trimethylsilyl)acetamide (BSA, 6.0 equiv.) and acetonitrile (4 L, 15 volumes) were charged into a 10 L reactor under nitrogen atmosphere at 15-20° C. The mixture was stirred for 40 min at 20° C. A solution of TMSOTf (212.9 g, 3.0 equiv.) in acetonitrile (1.3 L, 5 volumes) was charged dropwise over 0.5 hours maintain the internal temperature between 15-20° C. The solution was aged for 1 hour at 15-20° C. When process analysis (sample preparation 0.1 mL system+0.9 mL ACN+one drop of diisopropylethylamine) showed complete conversion of staring material the mixture was quenched with diisoproylethylamine (617 g, 15.0 equiv.) maintain a temperature between 5-10° C. The mixture was stirred for 20 minutes at 5-10° C., then a saturated aqueous NH₄Cl solution (2.6 L, 10 volumes) was charged maintaining a temperature between 5-10° C. The mixture was aged for an additional 30 minutes at this temperature. The aqueous phase (contained solids) was collected and was extracted with 2-MeTHF (520 ml, 2 volumes). The organic phases were combined and checked for water content by KF (KF: 9.18%), then dried with anhydrous Na₂SO₄ (500 g, 10.0 equiv.). The solids were removed by filtration and the filter cake was washed by acetonitrile (2×520 ml, 2 volumes). The filtrates were then dried with anhydrous Na₂SO₄ (500 g, 10.0 equiv.). After filtration, the filter cake was washed by acetonitrile (2×520 ml, 2 volumes) and water content was checked by KF (KF: 8.15%). The acetonitrile/2-MeTHF stream of 10 was used for next step directly. (The product was not stable to LCMS conditions).

### Preparation of Compound 14 (Free Acid)

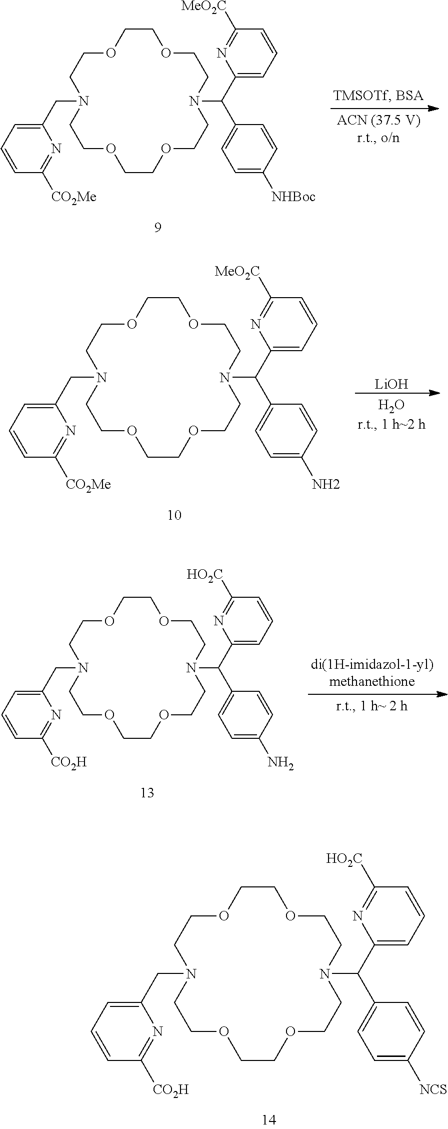

Methyl 6-((4-((tert-butoxycarbonyl)amino)phenyl)(16-((6-(methoxycarbonyl)pyridin-2-yl)methyl)-1,4,10,13-tetraoxa-7,16-diazacyclooctadecan-7-yl)methyl)picolinate (6.0 g, 1.0 equiv.) and BSA (9.7 g, 6.0 equiv.) and MeCN (120 mL, 20 volumes with respect to 9) were charged to a 500 mL reactor under nitrogen atmosphere at room temperature. A solution of TMSOTf (5.4 g, 2.3 equiv.) in MeCN (120 mL, 20 volumes) was added dropwise over 30 min at room temperature. The mixture was aged for overnight at room temperature. Analysis of the mixture (sample preparation 0.1 mL system+0.9 mL ACN+one drop of diisopropylethylamine) showed the reaction had reached completion. The mixture was quenched with diisoproylethylamine (15.4 g, 15.0 equiv.) maintain a temperature between 0-5° C. The mixture was stirred for 5 minutes at 0-5° C., then a saturated aqueous NH₄Cl solution (60 mL, 10 volumes) was charged dropwise maintaining a temperature between 0-5° C. The aqueous phase was removed by extraction and the organic phase was collected and used for next step directly. The organic phase was charged to 500 mL 3-necked round bottle bottom bottle, a solution of LiOH (1.15 g, 6.0 equiv.) in water (60 mL, 10 V) was added to the solution at room temperature. The solution was stirred for 1 hour at this temperature. Analysis of the mixture (sample preparation, 0.1 mL system+0.9 mL acetonitrile) showed not fully conversion. Another portion of LiOH (576 mg, 3.0 equiv.) was added and the solution was stirred for another 1 hour at room temperature. Analysis of the mixture (sample preparation, 0.1 mL system+0.9 mL acetonitrile) showed the reaction had reached completion. Then, TCDI (5.6 g, 3.9 equiv.) was added and the solution was stirred for 1 hour at room temperature. Analysis of the mixture (sample preparation, 0.1 mL system+0.9 mL acetonitrile) showed not fully conversion. Another portion of TCDI (2.8 g, 2.0 equiv.) was added and the solution was stirred for another 1 hour at room temperature. Analysis of the mixture (sample preparation, 0.1 mL system+0.9 mL acetonitrile) showed the reaction had reached completion. The reaction solution was separated by reversal phase Combi-Flash. Method: column C18, A solution H₂O (Containing 0.01% formic acid), B solution ACN. 5% to 35% in 40 min, flow (100 mL/min), product in 20 min-25 min. Collect a solution. The solution was concentrated to remove ACN and separated by reversal phase Combi-Flash again. Method: column C18, A solution H₂O, B solution ACN. 5% 10 min, 5% to 35% in 5 min 95% 10 min, flow (100 mL/min), product in 13 min-25 min. Collect a solution. The solution was concentrated under vacuum at <20° C. and dried by lyophilization. This result in 2.5 g (47% yield in 3 steps) compound 14 as a yellow solid. Compound 14 (6-((16-((6-carboxypyridin-2-yl)(4-isothiocyanatophenyl)methyl)-1,4,10,13-tetraoxa-7,16-diazacyclooctadecan-7-yl)methyl)picolinic acid) required storage at −80° C. LCMS: (ES, m/z): 666.3 [M+H]^{+ 1}H-NMR: (400 MHz, D₂O, ppm): 7.94-7.84 (m, 4H), 7.56-7.40 (m, 4H), 7.16-7.14 (m, 2H), 5.83 (s, 1H), 4.56 (s, 2H), 3.80-3.75 (m, 8H), 3.60-3.49 (m, 14H), 3.36-3.33 (m, 2H).

### Preparation of Compound 11 (Sodium Salt)

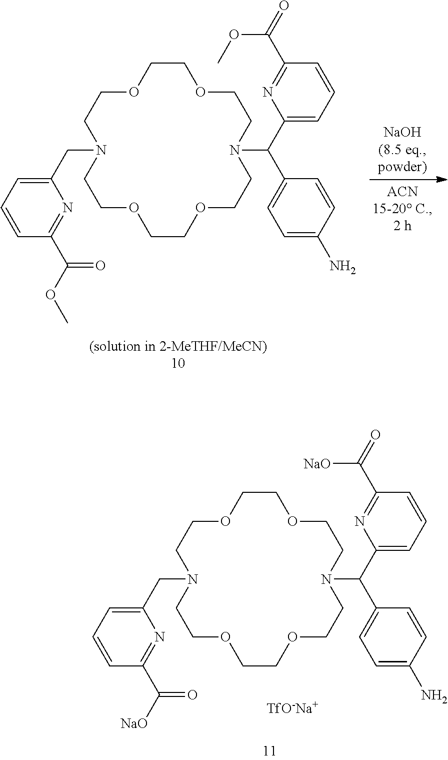

The prepared solution of compound 10 in ACN and 2-MeTHF was charged to a 1 0 L 4-necked reaction and the solution was cooled to 5-10° C. Powdered NaOH (56.9 g, 4.5 equiv.) was added maintaining the temperature between 5-10° C. The solution was stirred for 0.5 hours at 15-20° C. Analysis of the mixture (sample preparation, 0.1 mL system+0.9 mL acetonitrile) showed no conversion. Additional powdered NaOH (25.3 g, 2.0 equiv.) was added at 5-10° C. The solution was aged for an additional 0.5 hours at 15-20° C. A second IPC was analyzed and showed there was 50% conversion. A final charge of powdered NaOH (25.3 g, 2.0 equiv.) was added at 5-10° C. The mixture was stirred for additional 0.5 hours at 15-20° C. Analysis showed complete conversion of the starting material 10. The mixture was filtered, and the filter cake was washed by acetonitrile (2×520 ml, 2 volumes). The final solution (˜7.5 L, 28.8 volumes) was concentrated to 1-2 volumes maintaining a temperature between 15-20° C. The residue was then treated with acetonitrile (2 L, 7.7 volumes) and the water content was checked by KF (KF: 5.7%). The mixture was filtered, and the filter cake was washed by ACN (2×520 ml, 2 volumes). The solution was then concentrated to 1-2 V under vacuum at 15-20° C. The water content was again checked by KF (KF: 5.5%). The solution was diluted with acetonitrile (390 ml, 1.5 volumes), and was added dropwise over 0.5 hours into MTBE (2.6 L, 10 volumes) maintaining a temperature between 15-20° C. The solvents were decanted to leave a viscous oil which was redissolved in acetonitrile (520 ml, 2 volumes) and added into MTBE (2.6 L, 10 volumes). This process was repeated a further four times. To yield a viscous oil which was finally dissolved in acetonitrile (520 ml, 2 volumes) and dried, then concentrated at 15-20° C. under reduced pressure. Residual solvents were then removed by evaporation with an oil pump at 15-20° C. After drying 335 g of compound 11 was afforded as an off-yellow solid (QNMR: 70%, 87% overall yield over the two steps). LCMS (ES, m/z): 624.3 [M-TfONa-2Na+3H]^{+ 1}H-NMR (300 MHz, Methanol-d₄, ppm): δ 7.97 (dd, J=7.8, 2.1 Hz, 2H), 7.84 (t, J=7.7 Hz, 1H), 7.75 (t, J=7.8 Hz, 1H), 7.36 (dd, J=7.8, 1.1 Hz, 1H), 7.23 (d, J=7.7 Hz, 1H), 7.11 (d, J=8.5 Hz, 2H), 6.72 (d, J=8.5 Hz, 2H), 3.96 (s, 1H), 3.83-3.36 (m, 18H), 3.03-2.62 (m, 6H), 2.55 (d, J=14.3 Hz, 2H).

### Preparation of Compound 12 (TOPA-[C7]-phenylisothiocyanate Sodium Salt)

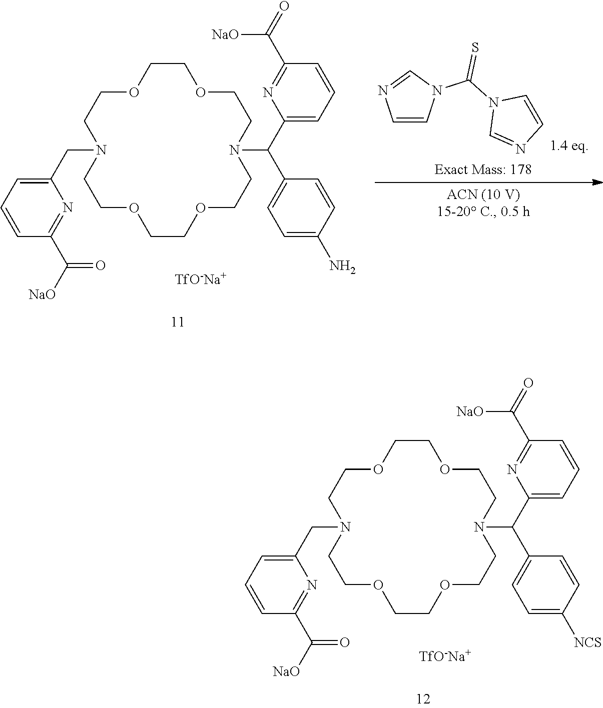

TCDI (68.7 g, 1.4 equiv.) and acetonitrile (2.6 L, 8 volumes) were charged to a 10 L reactor under nitrogen atmosphere at 15-20° C. A solution of compound 11 (330 g, Na⁺ salt, QNMR: 70%, 1.0 equiv.) in acetonitrile (660 mL, 2 volumes) was added dropwise over 30 min maintaining a temperature between 15-20° C. The mixture was aged for 0.5 hours at 15-20° C. Analysis of the mixture (sample preparation:30 μL system+300 μL ACN+a drop of water) showed the reaction had reached completion. The water content was checked by KF (KF: 0.19%). The system was dried and concentrated at 15-20° C. under reduced pressure. The resulting residue was dissolved in acetonitrile (945 ml, 2.9 volumes) and the water content was measured by KF (KF: 0.34%). isopropyl acetate (660 ml, 2 volumes) was charged to the solution over 40 minutes at 15-20° C. No nucleation was observed, and additional isopropyl acetate (6.6 L, 18 volumes) was charged dropwise slowly in 40 min at 15-20° C. leading to precipitation of the product 12 which was collected by filtration as an off yellow solid. The solid was dissolved in acetonitrile (330 ml, 1 volume) and IPAc (6.6 L, 20 volumes) was charged dropwise slowly in 40 min at 15-20° C. The mixture was filtered to yield 230 g of product as an off-yellow solid (LCAP: 80.99%, QNMR: 59%, 10% IPAc). The wet cake was dried under vacuum in 2 hours at 15-20° C., to give 224 g of crude 12 as an off-yellow solid (LCAP: 80.9%, QNMR: 60.4%, ˜6% IPAc). The crude 12 was redissolved in acetonitrile (330 ml, 1 volumes) and isopropyl acetate (412 ml, 1.25 volumes) was charged dropwise slowly in 40 min at 15-20° C. The resulting mixture was filtered, and 12 was collected (30.5 g, HPLC=60.9%, assay: 25.5%). The mother liquors were diluted with isopropyl acetate (6.6 L, 20 volumes) added over 40 minutes at 15-20° C. The mixture was filtered, and the cake was dried to afford 173.5 g of crude product 12 as an off-yellow solid (LCAP: 85.4%, QNMR: 66%, 3.9% IPAc, RRT1.19=3.9%). 190 g of crude 12 product was dissolved in 760 mL of acetonitrile:isopropyl acetate (2:1) and the mixture was passed through a silica gel column (380 g, 2×). The silica was flushed with acetonitrile:isopropyl acetate (2:1, 5.7 L) and then 12 L acetonitrile (very little product). Product containing fraction were the concentrated to afford 118 g of product 12 as an off-yellow solid (LCAP: 95%). The silica pad was then flushed with MeCN/H₂O (12 L, 10:1). The solvents were removed in vacuo to afford and additional 60 g crude 12 as an off-yellow solid which was dissolved in acetonitrile (1.5 L), stirred for 30 min then filtered. The mother liquor were then concentrated to afford 24 g of crude 12 as an off-yellow solid (LCAP=92%). crude 12 (118 g) and crude 12 (24 g) prepared as above were dissolved in acetonitrile (330 ml, 1 volume) and isopropyl acetate (6.6 L, 20 volumes) was charge dropwise over 40 min 15-20° C. The mixture was then filtered to afford 133 g of 12 product as off-yellow solid of suitable purity (LCAP: 95%, QNMR: 60.8%, 7.8% IPAc). Note, compound 12 required storage at −20° C. LCMS: (ES, m/z): 666.61 [M-TfONa-2Na+3H]^{+ 1}H-NMR: (400 MHz, methanol-d₄, ppm): δ 8.00 (ddd, J=13.8, 7.7, 1.0 Hz, 2H), 7.84 (dt, J=20.4, 7.7 Hz, 2H), 7.58-7.49 (m, 2H), 7.40 (dd, J=7.6, 1.0 Hz, 1H), 7.36-7.28 (m, 2H), 7.28-7.20 (m, 1H), 4.96 (hept, J=6.3 Hz, 1H), 3.96-3.88 (m, 1H), 3.83 (d, J=15.1 Hz, 1H), 3.70-3.52 (m, 11H), 3.55-3.39 (m, 4H), 3.07-2.73 (m, 6H), 2.62 (dt, J=15.1, 3.6 Hz, 2H).

### Example 20

### TOPA-[C7]-phenylthiourea-h11B6 Antibody Conjugate

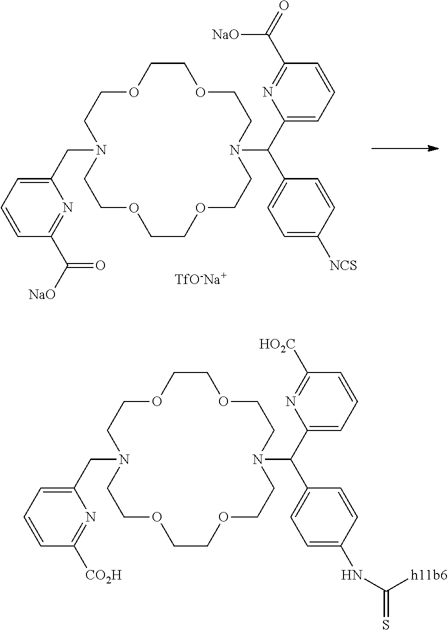

(In the TOPA-[C7]-phenylthiourea-h11B6 Antibody Conjugate depicted above, the structure does not show the lysine residue of h11b6 that is linked to the phenylthiourea moiety.)

### TOPA-[C7]-phenylthiourea modification of mAb

h11b6 mAb (10.2 mg/ml) was diluted to 1 mg/ml in 10 mM sodium acetate pH 5.2 buffer. Directly prior to conjugation, pH was adjusted to pH 9 with sodium bicarbonate buffer (VWR 144-55-8). pH was confirmed with pH paper. Then, 10× molar excess of disodium salt TOPA-[C7]-phenylisothiocyanate sodium salt (50 mM stock dissolved in water) was added to the h11b6 mAb, and the mixture of antibody and TOPA-[C7]-phenylisothiocyanate sodium salt was incubated at room temperature without shaking for approximately 1 hour. The addition of TOPA-[C7]-phenylisothiocyanate sodium salt was monitored by intact mass ESI-TOF LC-MS on an Agilent® G224 instrument until the CAR value was between 1.5-2.0. The mixture was then immediately quenched by addition of 1M Tris pH 8.5 (Teknova T1085) to a final concentration of 100 mM. Excess free chelator was removed by desalting the reaction into 10 mM sodium acetate pH 5.2 using a 7K Zeba® desalting column. To confirm no excess chelator was present, 3× rounds of sample dilution to 15 mls followed by concentration to 1 ml using a 50,000 MWCO Amicon concentrator device was performed. Sample was then concentrated to its final concentration for radiolabeling. The final conjugate was confirmed to be monomeric by analytical size exclusion chromatography on a Tosoh TSKgel G3000SWxl 7.8 mm×30 cm, 5 u column; column temperature: room temperature; the column was eluted 0.2M sodium phosphate pH 6.8; flow rate: 0.8 mL/min; 18 min run; injection volume: 18 ul.

### Example 21

### Ac-225 labeled TOPA-[C7]-phenylthiourea-h11B6 Antibody Conjugate

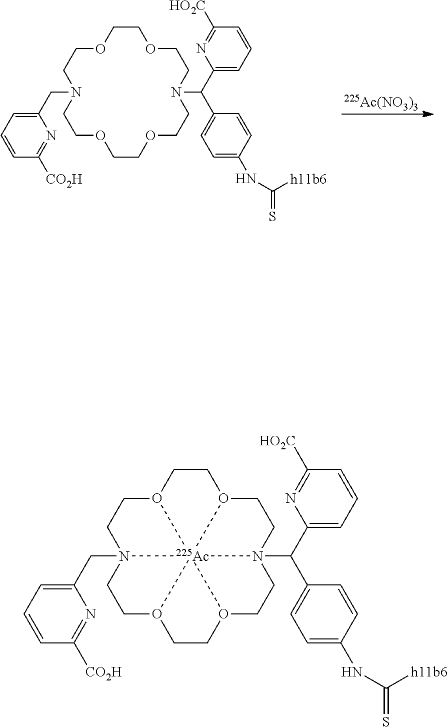

(In the Ac-225 labeled TOPA-[C7]-phenylthiourea-h11B6 Antibody Conjugate depicted above, the structure does not show the lysine residue of h11b6 that is linked to the phenylthiourea moiety.)

### (i) Labeling of TOPA-[C7]-phenylthiourea-h11B6 with Ac-225 in 3MNaOAc Buffer

To a solution of NaOAc (3 M in H₂O, 60 L) in a plastic vial were added sequentially Ac-225 (10 mCi/mL in 0.1 N HCl, 15 μL) and TOPA-[C7]-phenylthiourea-h11B6 (1.13 mg/mL in 10 mM NaOAc pH=5.5, 441 μL, 0.5 mg). After mixing, the pH was ˜6.5 by pH paper. The vial was left standing still at 37° C. for 2 hr.

iTLC of the Labeling Reaction Mixture:

0.5 μL of the labeling reaction mixture was loaded onto an iTLC-SG, which was developed with 10 mM EDTA (pH 5-6). The dried iTLC-SG was left at room temperature for overnight before it was scanned on a Bioscan AR-2000 radio-TLC scanner. Under the elution conditions described herein, TOPA-[C7]-phenylthiourea-h11B6 bound Ac-225 stayed at the origin and any free Ac-225 would migrate with the solvent to the solvent front. Scanning of the iTLC showed 99.9% TOPA-[C7]-phenylthiourea-h11B6 bound Ac-225.

### DTPA Challenge of the Labeling Reaction Mixture:

0.5 uL of the labeling reaction mixture was also mixed with 10 mM DTPA (pH=6.5, 15 L) at 37° C. After 30 min, 10 μL of the mixture was spotted on iTLC-SG and developed by 10 mM EDTA. The dried iTLC-SG was left at room temperature for overnight before it was scanned on a Bioscan AR-2000 radio-TLC scanner. Under the elution conditions described herein, TOPA-[C7]-phenylthiourea-h11B6 chelated Ac-225 stayed at the origin and any free Ac-225 would migrate with the solvent to the solvent front. Scanning of the iTLC showed 99.7% TOPA-[C7]-phenylthiourea-h11B6 chelated Ac-225.

### Purification on PD10 Column:

The PD-10 resin was conditioned in NaOAc buffer solution by passing 5 mL×3 of NaOAc buffer (25 mM NaOAc, 0.04% PS-20, pH 5.5) through column and discarding the washings. The entire labeling reaction mixture was applied to the reservoir of the column and the eluate collected in pre-numbered plastic tubes. The reaction vial was washed with 0.2 mL×3 NaOAc buffer (25 mM NaOAc, 0.04% PS-20, pH 5.5) and the washings pipetted into the reservoir of the PD-10 column and the eluate collected. Each tube contained ˜1 mL of the eluate. Continued application of NaOAc buffer (25 mM NaOAc, 0.04% PS-20, pH 5.5) into the reservoir of the PD-10 column occurred until a total elution volume of 10 mL was reached. The radiochemical purity of fractions collected were checked by iTLC: 10 μL of each collected fraction was spotted on iTLC-SG and developed with 10 mM EDTA. The dried iTLC-SG was left at room temperature for overnight before it was scanned on a Bioscan AR-2000 radio-TLC scanner. Pure fraction should have no radioactivity signal at the solvent front of the iTLC-SG.

### DTPA Challenge of the Purified ²²⁵Ac-TOPA-[C7]-Phenylthiourea-h11B6:

10 μL of fraction #3 collected after PD-10 column was mixed with 15 μL of 10 mM DTPA solution (pH 6.5), and incubated for 30 min. 10 μL of the mixture was loaded onto an iTLC-SG, which was developed with 10 mM EDTA and dried overnight. It was scanned on a Bioscan AR-2000 radio-TLC scanner. No radioactivity signal was observed at the solvent front of the iTLC-SG indicating that there was no free Ac-225 in the fraction #3.

### HPLC Analysis of the Purified ²²⁵Ac-TOPA-[C7]-Phenylthiourea-h11B6:

The fraction #3 collected after PD-10 column was analyzed by HPLC. HPLC method: Tosoh TSKgel G3000SWxl 7.8 mm×30 cm, 5 μm column; column temperature: room temperature; the column was eluted with DPBS buffer (X1, without calcium and magnesium); flow rate: 0.7 mL/min; 20 min run; injection volume: 40 μL. After HPLC, the fractions were collected in time intervals of 30 seconds or 1 minute. The collected HPLC fractions were left at room temperature overnight. The radioactivity in each of the collected fractions was counted in a gamma counter. The HPLC radio trace was constructed from the radioactivity in each HPLC fraction. HPLC radio trace showed a radioactive peak corresponding to the TOPA-[C7]-phenylthiourea-h11B6 peak on HPLC UV trace.

### (ii) Labeling of TOPA-[C7]-phenylthiourea-h11B6 at Higher Concentration with Ac-225 in 1.5MNaOAc Buffer

To a solution of NaOAc (1.5 M in H₂O with 0.04% PS-20, 63 L) in a plastic vial were added sequentially Ac-225 (10 mCi/mL in 0.1 N HCl, 10 μL) and TOPA-[C7]-phenylthiourea-h11B6 (9.36 mg/mL in 10 mM NaOAc pH=5.2, 0.04% PS-20, 36 μL, 337 μg). After mixing, the pH was ˜6.5 by pH paper. The vial was left standing still at 37° C. for 2 hr.

iTLC of the Labeling Reaction Mixture:

0.5 μL of labeling reaction mixture was then loaded onto an iTLC-SG, which was developed with 10 mM EDTA. The dried iTLC-SG was left at room temperature for overnight before it was scanned on a Bioscan AR-2000 radio-TLC scanner. Under the elution conditions described herein, TOPA-[C7]-phenylthiourea-h11B6 bounded Ac-225 stayed at the origin and any free Ac-225 would migrate with the solvent to the solvent front. Scanning of the iTLC showed 99.9% TOPA-[C7]-phenylthiourea-h11B6 bonded Ac-225.

### DTPA Challenge of the Labeling Reaction Mixture:

0.5 uL of the labeling reaction mixture was also mixed with 10 mM DTPA (pH=6.5, 15 L) at 37° C. After 30 min, 10 μL of the mixture was spotted on iTLC-SG and developed by 10 mM EDTA. The dried iTLC-SG was left at room temperature for overnight before it was scanned on a Bioscan AR-2000 radio-TLC scanner. Under the elution conditions described herein, TOPA-[C7]-phenylthiourea-h11B6 chelated Ac-225 stayed at the origin and any free Ac-225 would migrate with the solvent to the solvent front. Scanning of the iTLC showed 99.9% TOPA-[C7]-phenylthiourea-h11B6 chelated Ac-225.

### (iii) Labeling of TOPA-[C7]-phenylthiourea-h11B6 at Higher Concentration with Ac-225 in 1 MNaOAc Buffer

To a solution of NaOAc (1.0 M in H₂O with 0.04% PS-20, 63 L) in a plastic vial were added sequentially Ac-225 (10 mCi/mL in 0.1 N HCl, 10 μL) and TOPA-[C7]-phenylthiourea-h11B6 (9.36 mg/mL in 10 mM NaOAc pH=5.2, 0.04% PS-20, 36 μL, 337 μg). After mixing, the pH was ˜6.5 by pH paper. The vial was left standing still at 37° C. for 2 hr.

iTLC of the Labeling Reaction Mixture:

0.5 μL of labeling reaction mixture was then loaded onto an iTLC-SG, which was developed with 10 mM EDTA. The dried iTLC-SG was left at room temperature for overnight before it was scanned on a Bioscan AR-2000 radio-TLC scanner. Under the elution conditions described herein, TOPA-[C7]-phenylthiourea-h11B6 bound Ac-225 stayed at the origin and any free Ac-225 would migrate with the solvent to the solvent front. Scanning of the iTLC showed 99.9% TOPA-[C7]-phenylthiourea-h11B6 bonded Ac-225.

### DTPA Challenge of the Labeling Reaction Mixture:

0.5 uL of the labeling reaction mixture was also mixed with 10 mM DTPA (pH=6.5, 15 L) at 37° C. After 30 min, 10 μL of the mixture was spotted on iTLC-SG and developed by 10 mM EDTA. The dried iTLC-SG was left at room temperature for overnight before it was scanned on a Bioscan AR-2000 radio-TLC scanner. Under the elution conditions described herein, TOPA-[C7]-phenylthiourea-h11B6 chelated Ac-225 stayed at the origin and any free Ac-225 would migrate with the solvent to the solvent front. Scanning of the iTLC showed 99.9% TOPA-[C7]-phenylthiourea-h11B6 chelated Ac-225.

Labeling of TOPA-[C7]-Phenylthiourea-h11B6 at Higher Concentration with Ac-225 in 25 mM NaOAc with 0.4% Tween-20, pH 5.5:

To a solution of NaOAc (25 mM in H₂O with 0.04% PS-20, pH 5.5, 10 L) in a plastic vial were added sequentially Ac-225 (10 mCi/mL in 0.1 N HCl, 5 μL), TOPA-[C7]-phenylthiourea-h11B6 (10.4 mg/mL in 10 mM NaOAc pH=5.2, 16 μL, 166 μg) and NaOH (0.1 M, 5 μL). After mixing, the pH was ˜6.0 by pH paper. The vial was left standing still at 37° C. for 2 hr.

iTLC of the Labeling Reaction Mixture:

0.5 μL of labeling reaction mixture was then loaded onto an iTLC-SG, which was developed with 10 mM EDTA. The dried iTLC-SG was left at room temperature for overnight before it was scanned on a Bioscan AR-2000 radio-TLC scanner. Under the elution conditions described herein, TOPA-[C7]-phenylthiourea-h11B6 bound Ac-225 stayed at the origin and any free Ac-225 would migrate with the solvent to the solvent front. Scanning of the iTLC showed 99.9% TOPA-[C7]-phenylthiourea-h11B6 bonded Ac-225.

### DTPA Challenge of the Labeling Reaction Mixture:

0.5 uL of the labeling reaction mixture was also mixed with 10 mM DTPA (pH=6.5, 15 L) at 37° C. After 30 min, 10 μL of the mixture was spotted on iTLC-SG and developed by 10 mM EDTA. The dried iTLC-SG was left at room temperature for overnight before it was scanned on a Bioscan AR-2000 radio-TLC scanner. Under the elution conditions described herein, TOPA-[C7]-phenylthiourea-h11B6 chelated Ac-225 stayed at the origin and any free Ac-225 would migrate with the solvent to the solvent front. Scanning of the iTLC showed 99.8% TOPA-[C7]-phenylthiourea-h11B6 chelated Ac-225.

Reaction Conditions for Labeling of TOPA-[C7]-Phenylthiourea-h11B6 with Ac-225

| TOPA-[C7]- | Buffer for | Radiochemical | |
|---|---|---|---|
| phenylthiourea- | Labeling | yield | DTPA |
| h11B6 in buffer | reaction | (iTLC) | challenge |
| 1.13 mg/mL in 10 | 3M NaOAc | >99% | >99% |
| mM NaOAc pH = 5.5 | | | |
| 9.36 mg/mL in 10 | 1.5M NaOAc, | >99% | >99% |
| mM NaOAc, pH = 5.2; | 0.04% PS-20 | | |
| 0.04% PS-20 | | | |
| 9.36 mg/mL in 10 | 1.0M NaOAc, | >99% | >99% |
| mM NaOAc, pH = 5.2; | 0.04% PS-20 | | |
| 0.04% PS-20 | | | |
| 10.4 mg/mL in 10 | 25 mM in | >99% | >99% |
| mM NaOAc, pH = 5.2 | NaOAc, | | |
| | 0.04% PS-20, | | |
| | pH 5.5 | | |

### Example 22—Generation of Anti-KLK2 Antibodies

### Antibody Generation Using Transgenic Mice (Ablexis®) and Transgenic Rats (OmniRat®) Expressing Human Immunoglobulin Loci

The OmniRat® contains a chimeric human/rat IgH locus (comprising 22 human VHs, all human D and JH segments in natural configuration linked to the rat CH locus) together with fully human IgL loci (12 Vκs linked to Jκ-Cκ and 16 Vλs linked to JD-CU) (see, e.g., Osborn, et al., J Immunol, 2013, 190(4): 1481-90). Accordingly, the rats exhibit reduced expression of rat immunoglobulin, and in response to immunization, the introduced human heavy and light chain transgenes undergo class switching and somatic mutation to generate high affinity chimeric human/rat IgG monoclonal antibodies with fully human variable regions. The preparation and use of OmniRat®, and the genomic modifications carried by such rats, is described in International Publication No. WO14/093908.

Ablexis® mice generate antibodies having human variable domains linked to human CH1 and CL domains, chimeric human/mouse hinge regions, and mouse Fc regions. Ablexis Kappa Mouse and Lambda Mouse strains are distinguished by which of their heavy chains are human or mouse as noted below. Antibodies produced by the Kappa Mouse lack sequences derived from mouse VH, DH and JH exons and mouse Vκ, Jκ and Cκ exons. The endogenous mouse IgL is active in the Kappa Mouse. The human Igκ chains comprise approximately 90-95% of the naïve repertoire and mouse IgL chains comprise approximately 5-10% of the naïve repertoire in this strain. Antibodies produced by the Lambda Mouse lack sequences derived from mouse VH, DH and JH exons and mouse Vλ, Jλ and Cλ exons. The endogenous mouse Igκ is active in the Lambda Mouse. The human IgL chains comprise approximately 40% of the naïve repertoire and mouse Igκ chains comprise approximately 60% of the naïve repertoire. The preparation and use of Ablexis®, and the genomic modifications carried by such mice, is described in International Publication No. WO11/123708.

Ablexis® mice and OmniRats® rats were immunized with soluble full length KLK2 protein (human kallikrein-2 6-His protein, with an amino acid sequence of

(SEQ ID NO: 454)
VPLIEGRIVGGWECEKHSQPWQVAVYSHGWAHCGGVLVHPQWVLTAAHC
LKKNSQVWLGRHNLFEPEDTGQRVPVSHSFPHPLYNMSLLKHQSLRPDE
DSSHDLMLLRLSEPAKITDVVKVLGLPTQEPALGTTCYASGWGSIEPEE
FLRPRSLQCVSLHYSEKVTEFMLCAGLWTGGKDTCGGDSGGPLVCNGVL
QGITSWGPEPCALPEKPAVYTKVVHYRKWIKDTIAANPHHHHHH.

Lymphocytes from Ablexis mice and OniRats rats were extracted from lymph nodes and fusions performed by cohorts. Cells were combined and sorted for CD138 expression. Hybridoma screening was performed in high throughput miniaturized MSD format using soluble hK2 antigen. Approximately >300 samples were identified to be hK2 binders. The binding of >300 anti-hKLK2 supernatant samples to human KLK2 protein was measured by single cycle kinetics method by Biacore 8K SPR. Additionally the supernatant samples were tested for binding to human KLK3 protein as well. In parallel, supernatants were also tested for binding to KLK2 expressing cell lines VCap and negative cell line DU145 by Flow Cytometry. Selected cell binders were moved forward to scFv conversion in both VH-VL and VL/VH orientation and thermal stability tests as described above. KL2B413, KL2B30, KL2B53 and KL2B242 resulted from the Ablexis mice immunization campaign. KL2B467 and KL2B494 resulted from the OmniRat immunization campaign.

Antibodies generated through the various immunization and humanization campaigns described above were expressed in a fab format, a mAb format, a scFv format in the VH-linker-VL orientation or a scFv format in VL-linker-VH orientation and were further analyzed as described below. The linker sequence of SEQ ID NO: 7 described above was used to conjugate the VH/VL regions.

### Example 23. Structural Characterization of Anti KLK2 Antibodies

Sequences of antibody variable domains and scFv antibody fragments which showed highest performance in intracellular assay are provided herein. Variable domains were expressed in a Fab format, a scFv format in the VH-linker-VL orientation or a scFv format in VL-linker-VH orientation.

### Variable Domains VH, VL and CDRs

Table 3 shows the VH and VL amino acid sequences of selected anti-hK2 antibodies. Table 4 shows the Kabat HCDR1, HCDR2 and HCDR3 of selected anti-hK2 selected antibodies. Table 5 shows the Kabat LCDR1, LCDR2 and LCDR3 of the selected anti-hK2 antibodies. Table 6 shows the AbM HCDR1, HCDR2 and HCDR3 of selected anti-hK2 antibodies. Table 7 shows the AbM LCDR1, LCDR2 and LCDR3 of the anti-hK2. Table 8 summarizes the variable domain sequence and SEQ ID NO of selected hK2 antibodies. Table 9 shows the protein and DNA SEQ ID NOs for the VH and VL regions.

TABLE 3
VH and VL amino acid sequences of selected anti-hK2 antibodies.

| | | | VH | | | VL |
|---|---|---|---|---|---|---|
| | | | SEQ | | | SEQ |
| | | VH amino acid | ID | | VL amino acid | ID |
| Ab name | VH name | Sequence | NO: | VL name | sequence | NO: |
| m11B6 | m11B6_VH | DVQLQESGPGLV | 125 | m11B6_VL | DIVLTQSPASLAVS | 124 |
| | | KPSQSLSLTCTVT | | | LGQRATISCRASES | |
| | | GNSITSDYAWNW | | | VEYFGTSLMHWYR | |
| | | IRQFPGNRLEWM | | | QKPGQPPKLLIYAA | |
| | | GYISYSGSTTYSP | | | SNVESGVPARFSGS | |
| | | SLKSRFSITRDTS | | | GSGTDFSLNIQPVE | |
| | | KNQFFLQLNSVTP | | | EDDFSMYFCQQTR | |
| | | EDTATYFCATGY | | | KVPYTFGGGTKLEI | |
| | | YYGSGFWGQGTL | | | K | |
| | | VTVSS | | | | |
| h11B6 | hu11B6_ | QVQLQESGPGLV | 5 | hu11B6_VL | DIVLTQSPDSLAVS | 2 |
| | VH | KPSDTLSLTCAVS | | | LGERATINCKASES | |
| | | GNSITSDYAWNW | | | VEYFGTSLMHWYQ | |
| | | IRQPPGKGLEWIG | | | QKPGQPPKLLIYAA | |
| | | YISYSGSTTYNPS | | | SNRESGVPDRFSGS | |
| | | LKSRVTMSRDTS | | | GSGTDFTLTISSLQA | |
| | | KNQFSLKLSSVTA | | | EDVAVYYCQQTRK | |
| | | VDTAVYYCATGY | | | VPYTFGQGTKLEIK | |
| | | YYGSGFWGQGTL | | | | |
| | | VTVSS | | | | |
| HCF3- | HCF3_VH | QVQLQESGPGLV | 6 | LCD6_VL | DIVLTQSPDSLAVS | 1 |
| LCD6 | | KPSDTLSLTCAVS | | | LGERATINCKASES | |
| | | GNSITSDYAWNW | | | VEYFGTSLMHWYQ | |
| | | IRQFPGKGLEWIG | | | QKPGQPPKLLIYAA | |
| | | YISYSGSTTYNPS | | | SNRESGVPDRFSGS | |
| | | LKSRVTISRDTSK | | | GSGTDFTLTIQSVQ | |
| | | NQFSLKLSSVTPV | | | AEDVSVYFCQQTR | |
| | | DTAVYYCATGYY | | | KVPYTFGQGTKLEI | |
| | | YGSGFWGQGTLV | | | K | |
| | | TVSS | | | | |
| HCG5- | HCG5_VH | QVQLQESGPGLV | 4 | LCB7_VL | DIVLTQSPDSLAVS | 3 |
| LCB7 | | KPSDTLSLTCAVS | | | LGERATINCKASES | |
| | | GNSITSDYAWNW | | | VEYFGTSLMHWYQ | |
| | | IRQFPGKGLEWM | | | QKPGQPPKLLIYAA | |
| | | GYISYSGSTTYNP | | | SNRESGVPDRFSGS | |
| | | SLKSRVTISRDTS | | | GSGTDFTLTISSVQ | |
| | | KNQFSLKLSSVTP | | | AEDVAVYYCQQTR | |
| | | VDTAVYYCATGY | | | KVPYTFGQGTKLEI | |
| | | YYGSGFWGQGTL | | | K | |
| | | VTVSS | | | | |
| KL2B357 | KL2B357_ | QVQLQESGPGLV | 159 | KL2B357_ | DIVLTQSPDSLAVS | 160 |
| | VH | KPSQTLSLTCTVS | | VL | LGERATINCRASES | |
| | | GNSITSDYAWNW | | | VEYFGTSLMHWYQ | |
| | | IRQFPGKGLEWIG | | | QKPGQPPKLLIYAA | |
| | | YISYSGSTTYNPS | | | SNVESGVPDRFSGS | |
| | | LKSRVTISRDTSK | | | GSGTDFTLTISSLQA | |
| | | NQFSLKLSSVTAA | | | EDVAVYFCQQTRK | |
| | | DTAVYYCATGYY | | | VPYTFGGGTKVEIK | |
| | | YGSGFWGQGTLV | | | | |
| | | TVSS | | | | |
| KL2B358 | KL2B358_ | QVQLQESGPGLV | 161 | KL2B358_ | EIVLTQSPATLSLSP | 140 |
| | VH | PSQTLSLTCTVSGN | | VL | GERATLSCRASESV | |
| | | TSDYAWNWIRQPP | | | EYFGTSLMHWYQQ | |
| | | KGLEWIGYISYSGS | | | KPGQPPRLLIYAAS | |
| | | TYNPSLKSRVTISR | | | NVESGIPARFSGSG | |
| | | TSKNQFSLKLSSVT | | | SGTDFTLTISSVEPE | |
| | | ADTAVYYCATGY | | | DFAVYFCQQTRKV | |
| | | YGSGFWGQGTLVT | | | PYTFGGGTKVEIK | |
| | | SS | | | | |
| KL2B359 | KL2B359_ | QVQLQESGPGLV | 139 | KL2B359_ | EIVLTQSPATLSLSP | 140 |
| | VH | KPSQTLSLTCTVS | | VL | GERATLSCRASESV | |
| | | GNSITSDYAWNW | | | EYFGTSLMHWYQQ | |
| | | IRQFPGKRLEWIG | | | KPGQPPRLLIYAAS | |
| | | YISYSGSTTYNPS | | | NVESGIPARFSGSG | |
| | | LKSRVTISRDTSK | | | SGTDFTLTISSVEPE | |
| | | NQFSLKLSSVTAA | | | DFAVYFCQQTRKV | |
| | | DTAVYYCATGYY | | | PYTFGGGTKVEIK | |
| | | YGSGFWGQGTLV | | | | |
| | | TVSS | | | | |
| KL2B360 | KL2B360_ | QVQLQESGPGLV | 159 | KL2B360_ | EIVLTQSPATLSLSP | 140 |
| | VH | KPSQTLSLTCTVS | | VL | GERATLSCRASESV | |
| | | GNSITSDYAWNW | | | EYFGTSLMHWYQQ | |
| | | IRQFPGKGLEWIG | | | KPGQPPRLLIYAAS | |
| | | YISYSGSTTYNPS | | | NVESGIPARFSGSG | |
| | | LKSRVTISRDTSK | | | SGTDFTLTISSVEPE | |
| | | NQFSLKLSSVTAA | | | DFAVYFCQQTRKV | |
| | | DTAVYYCATGYY | | | PYTFGGGTKVEIK | |
| | | YGSGFWGQGTLV | | | | |
| | | TVSS | | | | |
| KL2B413 | KL2B413_ | EVQLVESGGGLV | 137 | KL2B413_ | EIVLTQSPSFLSASV | 138 |
| | VH | QPGGSLRLSCAAS | | VL | GDRVTITCRASQGI | |
| | | GFTFSSYWMTWV | | | SSYLSWYQQKPGK | |
| | | RQAPGKGLEWV | | | APKLLIYATSTLQS | |
| | | ANIKQDGSERYY | | | GVPSRFSGSGSGTE | |
| | | VDSVKGRFTISRD | | | FTLTISSLQPEDFAT | |
| | | NAKNSLYLQMNS | | | YYCQQLNSYPRTF | |
| | | LRAEDTAVYYCA | | | GQGTKVEIK | |
| | | RDQNYDILTGHY | | | | |
| | | GMDVWGQGTTV | | | | |
| | | TVSS | | | | |
| KL2B30 | KL2B30_ | QVQLQESGPGLV | 162 | KL2B30_VL | DIQMTQSPSFLSAS | 163 |
| | VH | KPSETLSLTCTVS | | | VGDRVTITCRASQG | |
| | | GGSISSYYWSWIR | | | ISSYLAWYQQKPG | |
| | | QPPGKGLEWIGYI | | | KAPKFLIYAASTLQ | |
| | | YYSGSTNYNPSL | | | SGVPSRFSGSGSGT | |
| | | KSRVTISVDTSKN | | | EFTLTISSLQPEDFA | |
| | | QFSLKLSSVTAAD | | | TYYCQQLNSYPLTF | |
| | | TAVYYCAGTTIF | | | GGGTKVEIK | |
| | | GVVTPNFYYGMD | | | | |
| | | VWGQGTTVTVSS | | | | |
| KL2B53 | KL2B53_ | EVQLVESGGGVV | 164 | KL2B53_VL | DIVMTQSPSSLSAS | 165 |
| | HV | QPGRSLRLSCVAS | | | VGDRVTITCRASQD | |
| | | GFTFSSYDIHWVR | | | ISNYLAWYQQKPG | |
| | | QAPGKGLEWVAI | | | KVPKFLIYAASTLH | |
| | | ISYDGSKKDYTDS | | | SGVPSRFSGSGSGT | |
| | | VKGRFTISRDNSK | | | DFTLTISSLQPEDVA | |
| | | NTLYLQMDSLRV | | | TYYCQKYNSAPYT | |
| | | ED SAVYSCARES | | | FGQGTRLEIK | |
| | | GWSHYYYYGMD | | | | |
| | | VWGQGTMVTVS | | | | |
| | | S | | | | |
| KL2B242 | KL2B242_ | QVQLQESGPGLV | 166 | KL2B242_ | SYELTQPPSVSVSP | 167 |
| | VH | KPSETLSLTCTVS | | VL | GETASITCSGDQLG | |
| | | GGSISSYYWSWL | | | ENYACWYQQKPG | |
| | | RQPAGSGLEWIG | | | QSPVLVIYQDSKRP | |
| | | RLYVSGFTNYNP | | | SGIPERFSGSNSGNT | |
| | | SLKSRVTLSLDPS | | | ATLTISGTQALDEA | |
| | | RNQLSLKLSSVTA | | | DYYCQAWDNSIVV | |
| | | ADTAVYYCAGDS | | | FGGGTKLTVL | |
| | | GNYWGWFDPWG | | | | |
| | | QGTLVTVSS | | | | |
| KL2B467 | KL2B467_ | QVQLVESGGGVV | 168 | KL2B467_ | QSVLTQPPSVSVAP | 169 |
| | VH | QPGRSLRLSCAAS | | VL | GQTASITCGGDNIG | |
| | | GFTFSYYGMHW | | | SKSVHWYQQKPGQ | |
| | | VRQAPGKGLEW | | | APVLVVYDNSDRP | |
| | | VAFISYDGSNKY | | | SGIPERFSGSNSGTT | |
| | | YADSVKGRFTISR | | | ATLTISRVEAGDEA | |
| | | DNSKNTLYLQMN | | | DYYCQVWDSSSDH | |
| | | SLRAEDTAVYYC | | | PVVFGGGTKVTV | |
| | | AHLPYSGSYWAF | | | | |
| | | DYWGQGTQVTV | | | | |
| | | SS | | | | |
| KL2B494 | KL2B494_ | QVQLVESGGGLV | 204 | KL2B494_ | SSELTQPPSVSVAP | 205 |
| | VH | QPGGSLRLSCAAS | | VL | GQTARITCGGNNIG | |
| | | GFTFSHYAMSWV | | | SKSVHWYQQKPGQ | |
| | | RQAPGKGLEWVS | | | APVLVVYDDSDRP | |
| | | TIGGSGGSTYYA | | | SGIPERFSGSNSGNT | |
| | | DSVKGRFTISRDN | | | ATLTISRVEAGDEA | |
| | | SKNTLYLQMNSL | | | DYYCQVWDSSSDH | |
| | | RAEDTAVYYCAK | | | VVFGGGTKLTVL | |
| | | PHIVMVTALLYD | | | | |
| | | GMDVWGQGTMV | | | | |
| | | TVSS | | | | |
| KL2B242LC_ | KL2B242_ | QVQLQESGPGLV | 166 | KL2B242LC_ | SYELTQPPSVSVSP | 444 |
| C33S | VH | KPSETLSLTCTVS | | C33S_VL | GETASITCSGDQLG | |
| | | GGSISSYYWSWL | | | ENYASWYQQKPGQ | |
| | | RQPAGSGLEWIG | | | SPVLVIYQDSKRPS | |
| | | RLYVSGFTNYNP | | | GIPERFSGSNSGNT | |
| | | SLKSRVTLSLDPS | | | ATLTISGTQALDEA | |
| | | RNQLSLKLSSVTA | | | DYYCQAWDNSIVV | |
| | | ADTAVYYCAGDS | | | FGGGTKLTVL | |
| | | GNYWGWFDPWG | | | | |
| | | QGTLVTVSS | | | | |

TABLE 4
Kabat HCDR1, HCDR2 and HCDR3 amino acid sequences of selected anti-KLK2 antibodies.

| | Kabat HCDR1 | | Kabat HCDR2 | | Kabat HCDR3 | |
|---|---|---|---|---|---|---|
| | | SEQ | | SEQ | | SEQ |
| | | ID | | ID | | ID |
| Ab name | Sequence | NO | Sequence | NO | Sequence | NO |
| m11B6 | SDYAWN | 63 | YISYSGSTTYSPSLKS | 64 | GYYYGSGF | 66 |
| hu11B6 | SDYAWN | 63 | YISYSGSTTYNPSLKS | 65 | GYYYGSGF | 66 |
| HCF3- | SDYAWN | 63 | YISYSGSTTYNPSLKS | 65 | GYYYGSGF | 66 |
| LCD6 | | | | | | |
| HCG5- | SDYAWN | 63 | YISYSGSTTYNPSLKS | 65 | GYYYGSGF | 66 |
| LCB7 | | | | | | |
| KL2B357 | SDYAWN | 63 | YISYSGSTTYNPSLKS | 65 | GYYYGSGF | 66 |
| KL2B358 | SDYAWN | 63 | YISYSGSTTYNPSLKS | 65 | GYYYGSGF | 66 |
| KL2B359 | SDYAWN | 63 | YISYSGSTTYNPSLKS | 65 | GYYYGSGF | 66 |
| KL2B360 | SDYAWN | 63 | YISYSGSTTYNPSLKS | 65 | GYYYGSGF | 66 |
| KL2B413 | SYWMT | 141 | NIKQDGSERYYVDSV | 142 | DQNYDILTGHY | 143 |
| | | | KG | | GMDV | |
| KL2B30 | SYYWS | 170 | YIYYSGSTNYNPSLKS | 171 | TTIFGVVTPNFY | 172 |
| | | | | | YGMDV | |
| KL2B53 | SYDIH | 176 | IISYDGSKKDYTDSVK | 177 | ESGWSHYYYY | 178 |
| | | | G | | GMDV | |
| KL2B242 | SYYWS | 170 | RLYVSGFTNYNPSLKS | 183 | DSGNYWGWFD | 184 |
| | | | | | P | |
| KL2B467 | YYGMH | 188 | FISYDGSNKYYADSV | 189 | LPYSGSYWAFD | 190 |
| | | | KG | | Y | |
| KL2B494 | HYAMS | 206 | TIGGSGGSTYYADSVK | 207 | PHIVMVTALLY | 208 |
| | | | G | | DGMDV | |

TABLE 5
Kabat LCDR1, LCDR2 and LCDR3 amino acid sequences of selected anti-hK2 antibodies.

| | Kabat LCDR1 | | Kabat LCDR2 | | Kabat LCDR3 | |
|---|---|---|---|---|---|---|
| | | SEQ | | SEQ | | SEQ |
| | | ID | | ID | | ID |
| Ab name | Sequence | NO | Sequence | NO | Sequence | NO |
| m11B6 | RASESVEYFGTSLMH | 67 | AASNVES | 69 | QQTRKVPYT | 71 |
| hu11B6 | KASESVEYFGTSLMH | 68 | AASNRES | 70 | QQTRKVPYT | 71 |
| HCF3- | KASESVEYFGTSLMH | 68 | AASNRES | 70 | QQTRKVPYT | 71 |
| LCD6 | | | | | | |
| HCG5- | KASESVEYFGTSLMH | 68 | AASNRES | 70 | QQTRKVPYT | 71 |
| LCB7 | | | | | | |
| KL2B357 | RASESVEYFGTSLMH | 67 | AASNVES | 69 | QQTRKVPYT | 71 |
| KL2B358 | RASESVEYFGTSLMH | 67 | AASNVES | 69 | QQTRKVPYT | 71 |
| KL2B359 | RASESVEYFGTSLMH | 67 | AASNVES | 69 | QQTRKVPYT | 71 |
| KL2B360 | RASESVEYFGTSLMH | 67 | AASNVES | 69 | QQTRKVPYT | 71 |
| KL2B413 | RASQGISSYLS | 144 | ATSTLQS | 145 | QQLNSYPRT | 146 |
| KL2B30 | RASQGISSYLA | 173 | AASTLQS | 174 | QQLNSYPLT | 175 |
| KL2B53 | RASQDISNYLA | 179 | AASTLHS | 180 | QKYNSAPYT | 181 |
| KL2B242 | SGDQLGENYAC | 185 | QDSKRPS | 186 | QAWDNSIVV | 187 |
| KL2B467 | GGDNIGSKSVH | 191 | DNSDRPS | 192 | QVWDSSSDH | 193 |
| | | | | | PVV | |
| KL2B494 | GGNNIGSKSVH | 182 | DDSDRPS | 470 | QVWDSSSDH | 209 |
| | | | | | VV | |

TABLE 6
AbM HCDR1, HCDR2 and HCDR3 amino acid sequences of selected anti-hK2 antibodies.

| | AbM HCDR1 | | AbM HCDR2 | | AbM HCDR3 | |
|---|---|---|---|---|---|---|
| | | SEQ | | SEQ | | SEQ |
| | | ID | | ID | | ID |
| Ab name | Sequence | NO | Sequence | NO | Sequence | NO |
| m11B6 | GNSITSDYA | 72 | YISYSGSTT | 73 | GYYYGSGF | 66 |
| | WN | | | | | |
| hu11B6 | GNSITSDYA | 72 | YISYSGSTT | 73 | GYYYGSGF | 66 |
| | WN | | | | | |
| HCF3- | GNSITSDYA | 72 | YISYSGSTT | 73 | GYYYGSGF | 66 |
| LCD6 | WN | | | | | |
| HCG5- | GNSITSDYA | 72 | YISYSGSTT | 73 | GYYYGSGF | 66 |
| LCB7 | WN | | | | | |
| KL2B357 | GNSITSDYA | 72 | YISYSGSTT | 73 | GYYYGSGF | 66 |
| | WN | | | | | |
| KL2B358 | GNSITSDYA | 72 | YISYSGSTT | 73 | GYYYGSGF | 66 |
| | WN | | | | | |
| KL2B359 | GNSITSDYA | 72 | YISYSGSTT | 73 | GYYYGSGF | 66 |
| | WN | | | | | |
| KL2B360 | GNSITSDYA | 72 | YISYSGSTT | 73 | GYYYGSGF | 66 |
| | WN | | | | | |
| KL2B413 | GFTFSSYWM | 147 | NIKQDGSER | 148 | DQNYDILTGHYGM | 143 |
| | T | | Y | | DV | |
| KL2B30 | GGSISSYYW | 194 | YIYYSGSTN | 195 | TTIFGVVTPNFYYG | 172 |
| | S | | | | MDV | |
| KL2B53 | GFTFSSYDIH | 196 | IISYDGSKK | 197 | ESGWSHYYYYGM | 178 |
| | | | D | | DV | |
| KL2B242 | GGSISSYYW | 198 | RLYVSGFTN | 199 | DSGNYWGWFDP | 184 |
| | S | | | | | |
| KL2B467 | GFTFSYY | 200 | FISYDGSNK | 201 | LPYSGSYWAFDY | 190 |
| | | | Y | | | |
| KL2B494 | GFTFSHYAM | 216 | TIGGSGGST | 217 | PHIVMVTALLYDG | 218 |
| | S | | YY | | MDV | |

TABLE 7
AbM LCDR1, LCDR2 and LCDR3 amino acid sequences of selected anti-hK2 antibodies.

| | AbM LCDR1 | | AbM LCDR2 | | AbM LCDR3 | |
|---|---|---|---|---|---|---|
| | | SEQ | | SEQ | | SEQ |
| | | ID | | ID | | ID |
| Ab name | Sequence | NO | Sequence | NO | Sequence | NO |
| m11B6 | RASESVEYFGTSL | 67 | AASNVES | 69 | QQTRKVPYT | 71 |
| | MH | | | | | |
| hu11B6 | KASESVEYFGTSL | 68 | AASNRES | 70 | QQTRKVPYT | 71 |
| | MH | | | | | |
| HCF3- | KASESVEYFGTSL | 68 | AASNRES | 70 | QQTRKVPYT | 71 |
| LCD6 | MH | | | | | |
| HCG5- | KASESVEYFGTSL | 68 | AASNRES | 70 | QQTRKVPYT | 71 |
| LCB7 | MH | | | | | |
| KL2B357 | RASESVEYFGTSL | 67 | AASNVES | 69 | QQTRKVPYT | 71 |
| | MH | | | | | |
| KL2B358 | RASESVEYFGTSL | 67 | AASNVES | 69 | QQTRKVPYT | 71 |
| | MH | | | | | |
| KL2B359 | RASESVEYFGTSL | 67 | AASNVES | 69 | QQTRKVPYT | 71 |
| | MH | | | | | |
| KL2B360 | RASESVEYFGTSL | 67 | AASNVES | 69 | QQTRKVPYT | 71 |
| | MH | | | | | |
| KL2B413 | RASQGISSYLS | 144 | ATSTLQS | 145 | QQLNSYPRT | 146 |
| KL2B30 | RASQGISSYLA | 173 | AASTLQS | 174 | QQLNSYPLT | 175 |
| KL2B53 | RASQDISNYLA | 179 | AASTLHS | 180 | QKYNSAPYT | 181 |
| KL2B242 | SGDQLGENYAC | 185 | QDSKRPS | 186 | QAWDNSIVV | 187 |
| KL2B467 | GGDNIGSKSVH | 191 | DNSDRPS | 192 | QVWDSSSDHPV | 193 |
| | | | | | V | |
| KL2B494 | GGNNIGSKSVH | 182 | DDSDRPS | 470 | QVWDSSSDHV | 209 |
| | | | | | V | |

TABLE 8
Amino acid sequences and SEQ ID NO summary of the variable domains of selected anti-hK2
antibodies

| | | | SEQ |
|---|---|---|---|
| Antibody | | | ID |
| name | Region | Amino acid sequence | NO: |
| m11B6 | HCDR1 | SDYAWN | 63 |
| | HCDR2 | YISYSGSTTYSPSLKS | 64 |
| | HCDR3 | GYYYGSGF | 66 |
| | LICDR1 | RASESVEYFGTSLMH | 67 |
| | LCDR2 | AASNVES | 69 |
| | LCDR3 | QQTRKVPYT | 71 |
| | VH | DVQLQESGPGLVKPSQSLSLTCTVTGNSITSDYAWN | 125 |
| | (m11B6_VH) | WIRQFPGNRLEWMGYISYSGSTTYSPSLKSRFSITRD | |
| | | TSKNQFFLQLNSVTPEDTATYFCATGYYYGSGFWG | |
| | | QGTLVTVSS | |
| | VL (m11B6_VL) | DIVLTQSPASLAVSLGQRATISCRASESVEYFGTSLM | 124 |
| | | HWYRQKPGQPPKLLIYAASNVESGVPARFSGSGSG | |
| | | TDFSLNIQPVEEDDFSMYFCQQTRKVPYTFGGGTKL | |
| | | EIK | |
| h11B6 | HCDR1 | SDYAWN | 63 |
| | HCDR2 | YISYSGSTTYNPSLKS | 65 |
| | HCDR3 | GYYYGSGF | 66 |
| | LICDR1 | KASESVEYFGTSLMH | 68 |
| | LCDR2 | AASNRES | 70 |
| | LCDR3 | QQTRKVPYT | 71 |
| | VH | QVQLQESGPGLVKPSDTLSLTCAVSGNSITSDYAWN | 5 |
| | (hu11B6_VH) | WIRQPPGKGLEWIGYISYSGSTTYNPSLKSRVTMSR | |
| | | DTSKNQFSLKLSSVTAVDTAVYYCATGYYYGSGF | |
| | | WGQGTLVTVSS | |
| | VL | DIVLTQSPDSLAVSLGERATINCKASESVEYFGTSLM | 2 |
| | (hu11B6_VL) | HWYQQKPGQPPKLLIYAASNRESGVPDRFSGSGSG | |
| | | TDFTLTISSLQAEDVAVYYCQQTRKVPYTFGQGTKL | |
| | | EIK | |
| HCF3- | HCDR1 | SDYAWN | 63 |
| LCD6 | HCDR2 | YISYSGSTTYNPSLKS | 65 |
| | HCDR3 | GYYYGSGF | 66 |
| | LICDR1 | KASESVEYFGTSLMH | 68 |
| | LCDR2 | AASNRES | 70 |
| | LCDR3 | QQTRKVPYT | 71 |
| | VH (HCF3_VH) | QVQLQESGPGLVKPSDTLSLTCAVSGNSITSDYAWN | 6 |
| | | WIRQFPGKGLEWIGYISYSGSTTYNPSLKSRVTISRD | |
| | | TSKNQFSLKLSSVTPVDTAVYYCATGYYYGSGFWG | |
| | | QGTLVTVSS | |
| | VL (LCD6_VL) | DIVLTQSPDSLAVSLGERATINCKASESVEYFGTSLM | 1 |
| | | HWYQQKPGQPPKLLIYAASNRESGVPDRFSGSGSG | |
| | | TDFTLTIQSVQAEDVSVYFCQQTRKVPYTFGQGTKL | |
| | | EIK | |
| HCG5- | HCDR1 | SDYAWN | 63 |
| LCB7 | HCDR2 | YISYSGSTTYNPSLKS | 65 |
| | HCDR3 | GYYYGSGF | 66 |
| | LICDR1 | KASESVEYFGTSLMH | 68 |
| | LCDR2 | AASNRES | 70 |
| | LCDR3 | QQTRKVPYT | 71 |
| | VH (HCG5_VH) | QVQLQESGPGLVKPSDTLSLTCAVSGNSITSDYAWN | 4 |
| | | WIRQFPGKGLEWMGYISYSGSTTYNPSLKSRVTISR | |
| | | DTSKNQFSLKLSSVTPVDTAVYYCATGYYYGSGFW | |
| | | GQGTLVTVSS | |
| | VL (LCB7_VL) | DIVLTQSPDSLAVSLGERATINCKASESVEYFGTSLM | 3 |
| | | HWYQQKPGQPPKLLIYAASNRESGVPDRFSGSGSG | |
| | | TDFTLTISSVQAEDVAVYYCQQTRKVPYTFGQGTK | |
| | | LEIK | |
| KL2B357 | HCDR1 | SDYAWN | 63 |
| | HCDR2 | YISYSGSTTYNPSLKS | 65 |
| | HCDR3 | GYYYGSGF | 66 |
| | LICDR1 | RASESVEYFGTSLMH | 67 |
| | LCDR2 | AASNVES | 69 |
| | LCDR3 | QQTRKVPYT | 71 |
| | VH | QVQLQESGPGLVKPSQTLSLTCTVSGNSITSDYAWN | 159 |
| | (KL2B357_VH) | WIRQFPGKGLEWIGYISYSGSTTYNPSLKSRVTISRD | |
| | | TSKNQFSLKLSSVTAADTAVYYCATGYYYGSGFW | |
| | | GQGTLVTVSS | |
| | VL | DIVLTQSPDSLAVSLGERATINCRASESVEYFGTSLM | 160 |
| | (KL2B_357_VL) | HWYQQKPGQPPKLLIYAASNVESGVPDRFSGSGSG | |
| | | TDFTLTISSLQAEDVAVYFCQQTRKVPYTFGGGTKV | |
| | | EIK | |
| KL2B358 | HCDR1 | SDYAWN | 63 |
| | HCDR2 | YISYSGSTTYNPSLKS | 65 |
| | HCDR3 | GYYYGSGF | 66 |
| | LICDR1 | RASESVEYFGTSLMH | 67 |
| | LCDR2 | AASNVES | 69 |
| | LCDR3 | QQTRKVPYT | 71 |
| | VH | QVQLQESGPGLVKPSQTLSLTCTVSGNSITSDYAWN | 161 |
| | (KL2B358_VH) | WIRQPPGKGLEWIGYISYSGSTTYNPSLKSRVTISRD | |
| | | TSKNQFSLKLSSVTAADTAVYYCATGYYYGSGFW | |
| | | GQGTLVTVSS | |
| | VL | EIVLTQSPATLSLSPGERATLSCRASESVEYFGTSLM | 140 |
| | (KL2B_358_VL) | HWYQQKPGQPPRLLIYAASNVESGIPARFSGSGSGT | |
| | | DFTLTISSVEPEDFAVYFCQQTRKVPYTFGGGTKVEI | |
| | | K | |
| KL2B359 | HCDR1 | SDYAWN | 63 |
| | HCDR2 | YISYSGSTTYNPSLKS | 65 |
| | HCDR3 | GYYYGSGF | 66 |
| | LICDR1 | RASESVEYFGTSLMH | 67 |
| | LCDR2 | AASNVES | 69 |
| | LCDR3 | QQTRKVPYT | 71 |
| | VH | QVQLQESGPGLVKPSQTLSLTCTVSGNSITSDYAWN | 139 |
| | (KL2B359_VH) | WIRQFPGKRLEWIGYISYSGSTTYNPSLKSRVTISRD | |
| | | TSKNQFSLKLSSVTAADTAVYYCATGYYYGSGFW | |
| | | GQGTLVTVSS | |
| | VL | EIVLTQSPATLSLSPGERATLSCRASESVEYFGTSLM | 140 |
| | (KL2B_359_VL) | HWYQQKPGQPPRLLIYAASNVESGIPARFSGSGSGT | |
| | | DFTLTISSVEPEDFAVYFCQQTRKVPYTFGGGTKVEI | |
| | | K | |
| KL2B360 | HCDR1 | SDYAWN | 63 |
| | HCDR2 | YISYSGSTTYNPSLKS | 65 |
| | HCDR3 | GYYYGSGF | 66 |
| | LICDR1 | RASESVEYFGTSLMH | 67 |
| | LCDR2 | AASNVES | 69 |
| | LCDR3 | QQTRKVPYT | 71 |
| | VH | QVQLQESGPGLVKPSQTLSLTCTVSGNSITSDYAWN | 159 |
| | (KL2B360_VH) | WIRQFPGKGLEWIGYISYSGSTTYNPSLKSRVTISRD | |
| | | TSKNQFSLKLSSVTAADTAVYYCATGYYYGSGFW | |
| | | GQGTLVTVSS | |
| | VL | EIVLTQSPATLSLSPGERATLSCRASESVEYFGTSLM | 140 |
| | (KL2B_360_VL) | HWYQQKPGQPPRLLIYAASNVESGIPARFSGSGSGT | |
| | | DFTLTISSVEPEDFAVYFCQQTRKVPYTFGGGTKVEI | |
| | | K | |
| KL2B413 | HCDR1 | SYWMT | 141 |
| | HCDR2 | NIKQDGSERYYVDSVKG | 142 |
| | HCDR3 | DQNYDILTGHYGMDV | 143 |
| | LICDR1 | RASQGISSYLS | 144 |
| | LCDR2 | ATSTLQS | 145 |
| | LCDR3 | QQLNSYPRT | 146 |
| | VH | EVQLVESGGGLVQPGGSLRLSCAASGFTFSSYWMT | 137 |
| | (KL2B413_VH) | WVRQAPGKGLEWVANIKQDGSERYYVDSVKGRFT | |
| | | ISRDNAKNSLYLQMNSLRAEDTAVYYCARDQNYDI | |
| | | LTGHYGMDVWGQGTTVTVSS | |
| | VL | EIVLTQSPSFLSASVGDRVTITCRASQGISSYLSWYQ | 138 |
| | (KL2B_413_VL) | QKPGKAPKLLIYATSTLQSGVPSRFSGSGSGTEFTLT | |
| | | ISSLQPEDFATYYCQQLNSYPRTFGQGTKVEIK | |
| KL2B30 | HCDR1 | SYYWS | 170 |
| | HCDR2 | YIYYSGSTNYNPSLKS | 171 |
| | HCDR3 | TTIFGVVTPNFYYGMDV | 172 |
| | LICDR1 | RASQGISSYLA | 173 |
| | LCDR2 | AASTLQS | 174 |
| | LCDR3 | QQLNSYPLT | 175 |
| | VH | QVQLQESGPGLVKPSETLSLTCTVSGGSISSYYWSW | 162 |
| | (KL2B30_VH) | IRQPPGKGLEWIGYIYYSGSTNYNPSLKSRVTISVDT | |
| | | SKNQFSLKLSSVTAADTAVYYCAGTTIFGVVTPNFY | |
| | | YGMDVWGQGTTVTVSS | |
| | VL | DIQMTQSPSFLSASVGDRVTITCRASQGISSYLAWY | 163 |
| | (KL2B30_VL) | QQKPGKAPKFLIYAASTLQSGVPSRFSGSGSGTEFTL | |
| | | TISSLQPEDFATYYCQQLNSYPLTFGGGTKVEIK | |
| KL2B53 | HCDR1 | SYDIH | 176 |
| | HCDR2 | IISYDGSKKDYTDSVKG | 177 |
| | HCDR3 | ESGWSHYYYYGMDV | 178 |
| | LICDR1 | RASQDISNYLA | 179 |
| | LCDR2 | AASTLHS | 180 |
| | LCDR3 | QKYNSAPYT | 181 |
| | VH | EVQLVESGGGVVQPGRSLRLSCVASGFTFSSYDIHW | 164 |
| | (KL2B53_VH) | VRQAPGKGLEWVAIISYDGSKKDYTDSVKGRFTISR | |
| | | DNSKNTLYLQMDSLRVED | |
| | | SAVYSCARESGWSHYYYYGMDVWGQGTMVTVSS | |
| | VL | DIVMTQSPSSLSASVGDRVTITCRASQDISNYLAWY | 165 |
| | (KL2B53_VL) | QQKPGKVPKFLIYAASTLHSGVPSRFSGSGSGTDFT | |
| | | LTISSLQPEDVATYYCQKYNSAPYTFGQGTRLEIK | |
| KL2B242 | HCDR1 | SYYWS | 170 |
| | HCDR2 | RLYVSGFTNYNPSLKS | 183 |
| | HCDR3 | DSGNYWGWFDP | 184 |
| | LICDR1 | SGDQLGENYAC | 185 |
| | LCDR2 | QDSKRPS | 186 |
| | LCDR3 | QAWDNSIVV | 187 |
| | VH | QVQLQESGPGLVKPSETLSLTCTVSGGSISSYYWSW | 166 |
| | (KL2B242_VH) | LRQPAGSGLEWIGRLYVSGFTNYNPSLKSRVTLSLD | |
| | | PSRNQLSLKLSSVTAADTAVYYCAGDSGNYWGWF | |
| | | DPWGQGTLVTVSS | |
| | VL | SYELTQPPSVSVSPGETASITCSGDQLGENYACWYQ | 167 |
| | (KL2B242_VL) | QKPGQSPVLVIYQDSKRPSGIPERFSGSNSGNTATLT | |
| | | ISGTQALDEADYYCQAWDNSIVVFGGGTKLTVL | |
| KL2B467 | HCDR1 | YYGMH | 188 |
| | HCDR2 | FISYDGSNKYYADSVKG | 189 |
| | HCDR3 | LPYSGSYWAFDY | 190 |
| | LICDR1 | GGDNIGSKSVH | 191 |
| | LCDR2 | DNSDRPS | 192 |
| | LCDR3 | QVWDSSSDHPVV | 193 |
| | VH | QVQLVESGGGVVQPGRSLRLSCAASGFTFSYYGMH | 168 |
| | (KL2B467_VH) | WVRQAPGKGLEWVAFISYDGSNKYYADSVKGRFTI | |
| | | SRDNSKNTLYLQMNSLRAEDTAVYYCAHLPYSGSY | |
| | | WAFDYWGQGTQVTVSS | |
| | VL | QSVLTQPPSVSVAPGQTASITCGGDNIGSKSVHWYQ | 169 |
| | (KL2B467_VL) | QKPGQAPVLVVYDNSDRPSGIPERFSGSNSGTTATL | |
| | | TISRVEAGDEADYYCQVWDSSSDHPVVFGGGTKVT | |
| | | V | |
| KL2B494 | HCDR1 | HYAMS | 206 |
| | HCDR2 | TIGGSGGSTYYADSVKG | 207 |
| | HCDR3 | PHIVMVTALLYDGMDV | 208 |
| | LCDR1 | GGNNIGSKSVH | 182 |
| | LCDR2 | DDSDRPS | 470 |
| | LCDR3 | QVWDSSSDHVV | 209 |
| | VH | QVQLVESGGGLVQPGGSLRLSCAASGFTFSHYAMS | 204 |
| | (KL2B494_VH) | WVRQAPGKGLEWVSTIGGSGGSTYYADSVKGRFTI | |
| | | SRDNSKNTLYLQMNSLRAEDTAVYYCAKPHIVMV | |
| | | TALLYDGMDVWGQGTMVTVSS | |
| | VL | SSELTQPPSVSVAPGQTARITCGGNNIGSKSVHWYQ | 205 |
| | (KL2B494_VL) | QKPGQAPVLVVYDDSDRPSGIPERFSGSNSGNTATL | |
| | | TISRVEAGDEADYYCQVWDSSSDHVVFGGGTKLTV | |
| | | L | |

TABLE 9
SEQ ID NO of Protein and DNA sequences of the VH and VL domains of selected hK2
antibodies.

| | VH | VL | VH | VL |
|---|---|---|---|---|
| | Protein | Protein | cDNA | cDNA |
| | SEQ ID | SEQ | SEQ ID | SEQ ID |
| Antibody | NO: | ID NO | NO: | NO: |
| m11B6 | 125 | 124 | 225 | 237 |
| hu11B6 | 5 | 2 | 226 | 238 |
| HCF3-LCD6 | 6 | 1 | 227 | 239 |
| HCG5-LCB7 | 4 | 3 | 228 | 240 |
| KL2B357 | 159 | 160 | 229 | 241 |
| KL2B358 | 161 | 140 | 230 | 242 |
| KL2B359 | 139 | 140 | 231 | 242 |
| KL2B360 | 159 | 140 | 229 | 242 |
| KL2B413 | 137 | 138 | 232 | 243 |
| KL2B30 | 162 | 163 | 233 | 244 |
| KL2B53 | 164 | 165 | 234 | 245 |
| KL2B242 | 166 | 167 | 235 | 246 |
| KL2B467 | 168 | 169 | 236 | 247 |
| KL2B494 | 204 | 205 | 263 | 271 |

SEQ ID NO: 225 (m11B6 VH cDNA)
GATGTGCAGCTTCAGGAGTCTGGACCCGGACTTGTTAAACCAAGTCAGTCTCTGTCCCTGAC
CTGTACCGTCACCGGCAACAGCATCACAAGCGATTACGCATGGAACTGGATCAGGCAGTTCC
CTGGAAATCGACTCGAATGGATGGGCTACATTTCATACTCCGGTTCAACCACTTACTCTCCAT
CCTTGAAATCTAGGTTCAGCATCACCCGTGATACCTCAAAGAACCAATTTTTTCTGCAACTG
AATAGCGTAACTCCAGAGGACACAGCCACATATTTCTGCGCCACTGGGTATTACTATGGCTC
AGGTTTCTGGGGTCAGGGCACTCTCGTCACCGTCAGCAGC
SEQ ID NO: 226 (hu11B6 VH cDNA)
CAGGTCCAACTGCAAGAGAGCGGACCGGGCCTGGTAAAGCCATCCGACACATTGTCCCTGA
CGTGTGCGGTAAGTGGAAACTCTATCACTAGCGACTATGCGTGGAATTGGATAAGACAACC
GCCGGGCAAGGGGCTGGAATGGATAGGATATATCAGCTATTCCGGTTCTACGACATACAATC
CTTCCCTGAAAAGCAGAGTCACTATGTCACGCGACACGTCCAAGAATCAGTTCTCATTGAAA
TTGTCATCCGTAACGGCCGTTGACACTGCGGTTTATTATTGCGCAACCGGATATTACTACGGC
TCTGGTTTTTGGGGACAGGGAACACTTGTTACTGTTAGTTCA
SEQ ID: NO 227 (HCF3-LCD6 VH cDNA)
CAGGTGCAGCTGCAGGAGAGCGGCCCAGGCCTGGTGAAGCCAAGCGACACCCTGAGCCTGA
CCTGCGCCGTGAGCGGCAACAGCATCACCAGCGACTACGCCTGGAACTGGATCCGCCAGTTC
CCAGGCAAGGGCCTGGAGTGGATCGGCTACATCAGCTACAGCGGCAGCACCACCTACAACC
CAAGCCTGAAGAGCCGCGTCACCATCAGCCGCGACACCAGCAAGAACCAGTTCAGCCTGAA
GCTGAGCAGCGTGACCCCTGTGGACACCGCCGTGTACTACTGCGCCACCGGCTACTACTACG
GCAGCGGCTTCTGGGGCCAGGGCACCCTGGTGACCGTGAGCAGC
SEQ ID NO: 228 (HCG5-LCB7 VH cDNA)
CAGGTGCAGCTGCAGGAGAGCGGCCCAGGCCTGGTGAAGCCAAGCGACACCCTGAGCCTGA
CCTGCGCCGTGAGCGGCAACAGCATCACCAGCGACTACGCCTGGAACTGGATCCGCCAGTTC
CCAGGCAAGGGCCTGGAGTGGATGGGCTACATCAGCTACAGCGGCAGCACCACCTACAACC
CAAGCCTGAAGAGCCGCGTCACCATCAGCCGCGACACCAGCAAGAACCAGTTCAGCCTGAA
GCTGAGCAGCGTGACCCCTGTGGACACCGCCGTGTACTACTGCGCCACCGGCTACTACTACG
GCAGCGGCTTCTGGGGCCAGGGCACCCTGGTGACCGTGAGCAGC
SEQ ID NO: 229 (KL2B357, KL2B360 VH cDNA)
CAGGTTCAGCTGCAAGAGTCTGGACCAGGCCTGGTCAAGCCCTCTCAGACCCTGTCTCTGAC
CTGTACCGTGTCCGGCAACTCCATCACCTCTGACTACGCCTGGAACTGGATTCGGCAGTTCC
CTGGCAAGGGCCTTGAGTGGATCGGCTACATCTCCTACTCCGGTTCCACCACCTACAACCCC
AGCCTGAAGTCCCGGGTCACCATCTCCCGCGACACCTCCAAGAACCAGTTCTCCCTGAAGCT
GTCCTCCGTGACCGCTGCTGATACCGCCGTGTACTACTGTGCCACCGGCTACTACTACGGCTC
CGGCTTTTGGGGACAGGGCACACTGGTTACCGTGTCTAGT
SEQ ID NO: 230 (KL2B358 VH cDNA)
CAGGTTCAGCTGCAAGAGTCTGGACCAGGCCTGGTCAAGCCCTCTCAGACCCTGTCTCTGAC
CTGTACCGTGTCCGGCAACTCCATCACCTCTGACTACGCCTGGAACTGGATTCGGCAGCCAC
CTGGCAAGGGCCTTGAGTGGATCGGCTACATCTCCTACTCCGGTTCCACCACCTACAACCCC
AGCCTGAAGTCCCGGGTCACCATCTCCCGCGACACCTCCAAGAACCAGTTCTCCCTGAAGCT
GTCCTCCGTGACCGCTGCTGATACCGCCGTGTACTACTGTGCCACCGGCTACTACTACGGCTC
CGGCTTTTGGGGACAGGGCACACTGGTTACCGTGTCTAGT
SEQ ID NO: 231 (KL2B359 VH cDNA)
CAGGTTCAGCTGCAAGAGTCTGGACCAGGCCTGGTCAAGCCCTCTCAGACCCTGTCTCTGAC
CTGTACCGTGTCCGGCAACTCCATCACCTCTGACTACGCCTGGAACTGGATTCGGCAGTTCC
CTGGCAAGCGCCTTGAGTGGATCGGCTACATCTCCTACTCCGGTTCCACCACCTACAACCCC
AGCCTGAAGTCCCGGGTCACCATCTCCCGCGACACCTCCAAGAACCAGTTCTCCCTGAAGCT
GTCCTCCGTGACCGCTGCTGATACCGCCGTGTACTACTGTGCCACCGGCTACTACTACGGCTC
CGGCTTTTGGGGACAGGGCACACTGGTTACCGTGTCTAGT
SEQ ID NO: 232 (KL2B413 VH cDNA)
GAGGTGCAACTTGTGGAGAGCGGCGGAGGTCTGGTCCAACCCGGAGGAAGTCTCCGTCTCT
CCTGTGCTGCTAGTGGCTTCACTTTCAGCTCATATTGGATGACATGGGTGAGACAAGCCCCA
GGAAAGGGGCTCGAGTGGGTAGCTAACATTAAACAGGACGGCTCCGAACGGTACTATGTTG
ATTCTGTGAAGGGACGGTTCACTATATCCAGGGATAATGCAAAAAATTCACTCTATCTTCAA
ATGAACTCACTCAGAGCAGAGGACACTGCCGTGTATTATTGCGCCAGGGATCAAAATTATGA
CATACTGACCGGTCATTATGGAATGGATGTTTGGGGCCAGGGAACAACCGTTACCGTCTCAA
GT
SEQ ID NO:233 (KL2B30 VH cDNA)
CAGGTGCAGCTGCAGGAGTCGGGCCCAGGACTGGTGAAGCCTTCGGAGACCCTGTCCCTCA
CCTGCACTGTCTCTGGTGGCTCCATCAGTAGTTACTACTGGAGCTGGATCCGGCAGCCCCCA
GGGAAGGGACTGGAGTGGATTGGATATATCTATTACAGTGGGAGCACCAACTACAACCCCT
CCCTCAAGAGTCGAGTCACCATATCAGTAGACACGTCCAAGAACCAGTTCTCCCTGAAGCTG
AGCTCTGTGACCGCTGCGGACACGGCCGTGTATTACTGTGCGGGGACTACGATTTTTGGAGT
GGTTACCCCCAACTTCTACTACGGTATGGACGTCTGGGGCCAAGGGACCACGGTCACCGTCT
CCTCA
SEQ ID NO: 234 (KL2B53 VH cDNA)
GAGGTGCAGCTGGTGGAGTCTGGGGGAGGCGTGGTCCAGCCTGGGAGGTCCCTGAGACTCT
CCTGTGTAGCCTCTGGATTCACCTTCAGTAGTTATGACATACACTGGGTCCGCCAGGCTCCA
GGCAAGGGGCTGGAGTGGGTGGCAATTATTTCATATGATGGAAGTAAAAAAGACTATACAG
ACTCCGTGAAGGGCCGATTCACCATCTCCAGAGACAATTCCAAGAACACGCTGTATCTGCAA
ATGGACAGCCTGAGAGTTGAGGACTCGGCTGTGTATTCCTGTGCGAGAGAAAGTGGCTGGTC
CCACTACTACTATTACGGTATGGACGTCTGGGGCCAAGGGACAATGGTCACCGTCTCTTCA
SEQ ID NO: 235 (KL2B242 VH cDNA)
CAGGTGCAGCTGCAGGAGTCGGGCCCAGGACTGGTGAAGCCTTCGGAGACCCTGTCCCTCA
CCTGCACTGTCTCTGGTGGCTCCATCAGTAGTTACTATTGGAGCTGGCTCCGGCAGCCCGCC
GGGTCGGGACTGGAGTGGATTGGGCGTTTATATGTCAGTGGGTTCACCAACTACAACCCCTC
CCTCAAGAGTCGAGTCACCTTGTCACTAGACCCGTCCAGGAACCAGTTGTCCCTGAAACTGA
GTTCTGTGACCGCCGCGGACACGGCCGTATATTATTGTGCGGGAGATAGTGGGAACTACTGG
GGTTGGTTCGACCCCTGGGGCCAGGGAACCCTGGTCACCGTCTCCTCA
SEQ ID NO: 236 (KL2B467 VH cDNA)
CAGGTGCAGCTGGTGGAGTCTGGGGGAGGCGTGGTCCAGCCTGGGAGGTCCCTGAGACTCT
CCTGTGCAGCCTCTGGATTCACCTTCAGTTACTATGGCATGCACTGGGTCCGCCAGGCTCCA
GGCAAGGGGCTGGAGTGGGTGGCATTTATATCATATGATGGAAGTAATAAATACTATGCAG
ACTCCGTGAAGGGCCGATTCACCATCTCCAGAGACAATTCCAAGAACACGCTGTATCTGCAA
ATGAACAGCCTGAGAGCTGAGGACACGGCTGTGTATTACTGTGCCCACCTCCCTTATAGTGG
GAGCTACTGGGCCTTTGACTACTGGGGCCAGGGAACCCAGGTCACCGTCTCTTCA
SEQ ID NO: 263 (KL2B494 VH cDNA)
CAGGTGCAGCTGGTGGAGTCTGGGGGAGGCTTGGTACAGCCTGGGGGGTCCCTGAGACTCT
CCTGTGCAGCCTCTGGATTCACCTTTAGTCATTATGCCATGAGCTGGGTCCGCCAGGCTCCAG
GGAAGGGGCTGGAGTGGGTCTCAACTATTGGTGGTAGTGGTGGTAGCACATACTACGCAGA
CTCCGTGAAGGGCCGGTTCACCATCTCCAGAGACAATTCCAAGAACACGCTGTATCTGCAAA
TGAACAGCCTGAGAGCCGAGGACACGGCCGTATATTACTGTGCGAAACCTCATATTGTAATG
GTGACTGCTCTTCTCTACGACGGTATGGACGTCTGGGGCCAAGGGACAATGGTCACCGTCTC
CTCA
SEQ ID NO: 237 (m11B6 VL cDNA)
GACATTGTGCTGACACAGAGTCCAGCATCCTTGGCAGTATCTTTGGGGCAGCGGGCAACAAT
TTCATGCCGTGCATCTGAAAGTGTGGAGTATTTTGGAACTTCTCTTATGCACTGGTATCGCCA
GAAGCCTGGGCAGCCTCCCAAACTCCTTATATATGCCGCTTCCAACGTGGAGTCCGGAGTAC
CAGCACGCTTTTCCGGCTCTGGGTCCGGCACAGACTTTTCCCTCAATATCCAACCTGTTGAAG
AAGACGATTTTTCCATGTATTTTTGCCAACAGACACGCAAGGTTCCATATACATTCGGCGGC
GGCACTAAACTTGAGATCAAA
SEQ ID NO: 238 (hu11B6 VL cDNA)
GACATAGTCTTGACTCAGAGCCCGGATTCCCTTGCTGTGTCTCTGGGAGAACGAGCTACGAT
CAACTGCAAGGCAAGTGAATCCGTAGAATACTTCGGGACATCATTGATGCATTGGTATCAAC
AGAAACCGGGGCAACCGCCCAAATTGCTGATATATGCGGCTAGTAATAGAGAATCAGGAGT
ACCGGATAGGTTTAGTGGTTCAGGATCAGGTACAGATTTCACCCTGACAATAAGTAGCTTGC
AAGCCGAAGACGTAGCAGTGTATTACTGCCAACAAACCCGAAAGGTGCCATATACGTTTGG
ACAGGGTACAAAGTTGGAAATCAAA
SEQ ID NO: 239 (HCF3-LCD6 VL cDNA)
GACATCGTGCTGACCCAGAGCCCAGACAGCCTGGCCGTGAGCCTGGGCGAGCGCGCCACCA
TCAACTGCAAGGCCAGCGAGAGCGTGGAGTACTTCGGCACCAGCCTGATGCACTGGTACCA
GCAGAAGCCAGGCCAGCCACCAAAGCTGCTGATCTACGCTGCCAGCAACCGCGAGAGCGGC
GTGCCAGACCGCTTCAGCGGCAGCGGCAGCGGCACCGACTTCACCCTGACCATCCAGAGCG
TGCAGGCCGAGGACGTCTCCGTGTACTTCTGCCAGCAGACCCGCAAGGTGCCATACACCTTC
GGCCAGGGCACCAAGCTGGAGATCAAG
SEQ ID NO: 240 (HCG5-LCB7 VL cDNA)
GACATCGTGCTGACCCAGAGCCCAGACAGCCTGGCCGTGAGCCTGGGCGAGCGCGCCACCA
TCAACTGCAAGGCCAGCGAGAGCGTGGAGTACTTCGGCACCAGCCTGATGCACTGGTACCA
GCAGAAGCCAGGCCAGCCACCAAAGCTGCTGATCTACGCTGCCAGCAACCGCGAGAGCGGC
GTGCCAGACCGCTTCAGCGGCAGCGGCAGCGGCACCGACTTCACCCTGACCATCAGCAGCG
TGCAGGCCGAGGACGTCGCCGTGTACTACTGCCAGCAGACCCGCAAGGTGCCATACACCTTC
GGCCAGGGCACCAAGCTGGAGATCAAG
SEQ ID NO: 241 (KL2B357 VL cDNA)
GACATCGTGCTGACCCAGTCTCCAGACTCTCTGGCTGTGTCTCTGGGCGAGAGAGCCACCAT
CAACTGCAGAGCCTCCGAGTCCGTGGAATACTTCGGCACCTCTCTGATGCACTGGTACCAGC
AGAAGCCCGGCCAGCCTCCTAAGCTGCTGATCTACGCCGCCTCCAACGTGGAATCTGGCGTG
CCCGATAGATTTTCCGGCTCTGGCTCTGGCACCGACTTTACCCTGACCATCAGCTCTCTGCAG
GCCGAGGATGTGGCCGTGTACTTCTGTCAGCAGACCCGGAAGGTGCCCTACACATTTGGCGG
CGGAACAAAGGTGGAAATCAAG
SEQ ID NO: 242 (KL2B358, KL2B359, KL2B360 VL cDNA)
GAGATCGTGCTGACCCAGTCTCCTGCCACACTGTCACTGTCTCCAGGCGAGAGAGCCACCCT
CTCTTGTAGAGCCTCCGAGTCCGTGGAATACTTCGGCACCTCTCTGATGCACTGGTACCAGC
AGAAGCCCGGCCAGCCTCCTAGACTGCTGATCTACGCCGCCTCCAACGTCGAATCTGGCATC
CCCGCTAGATTCTCCGGCTCTGGCTCTGGCACAGACTTTACCCTGACCATCTCCTCCGTGGAA
CCCGAGGATTTCGCTGTGTACTTTTGCCAGCAGACCCGGAAGGTGCCCTACACATTTGGCGG
CGGAACAAAGGTGGAAATCAAG
SEQ ID NO: 243 (KL2B413 VL cDNA)
GAAATCGTACTGACCCAGTCCCCTTCTTTCTTGAGTGCATCAGTTGGGGATAGAGTGACCAT
TACTTGTAGAGCATCTCAAGGTATTTCTTCATACTTGTCTTGGTATCAACAAAAACCTGGCAA
GGCACCCAAACTCTTGATCTACGCCACCTCTACATTGCAAAGTGGGGTTCCTTCTAGGTTTTC
AGGCTCCGGCTCTGGTACCGAGTTCACCCTCACTATAAGCAGTCTCCAACCTGAAGATTTCG
CTACTTATTATTGTCAGCAGCTTAATTCTTATCCCCGAACCTTTGGTCAAGGAACTAAGGTCG
AGATCAAA
SEQ ID NO: 244 (KL2B30 VL cDNA)
GACATCCAGATGACCCAGTCTCCTTCCTTCCTGTCTGCATCTGTAGGAGACAGAGTCACCAT
CACTTGCCGGGCCAGTCAGGGCATTAGCAGTTATTTAGCCTGGTATCAGCAAAAACCAGGGA
AAGCCCCTAAGTTCCTGATCTATGCTGCATCCACTTTGCAAAGTGGGGTCCCATCAAGGTTC
AGCGGCAGTGGATCTGGGACAGAATTCACTCTCACAATCAGCAGCCTGCAGCCTGAAGATTT
TGCAACTTATTACTGTCAACAGCTTAATAGTTACCCTCTCACTTTCGGCGGAGGGACCAAGG
TGGAAATCAAA
SEQ ID NO: 245 (KL2B53 VL cDNA)
GACATCGTGATGACCCAGTCTCCATCCTCCCTGTCTGCATCTGTAGGAGACAGAGTCACCAT
CACTTGCCGGGCGAGTCAGGACATTAGCAATTATTTAGCCTGGTATCAGCAGAAACCAGGG
AAAGTTCCTAAGTTCCTGATCTATGCTGCATCCACTTTGCACTCTGGGGTCCCATCTCGGTTC
AGTGGCAGTGGATCTGGGACAGATTTCACTCTCACCATCAGCAGCCTGCAGCCTGAAGATGT
TGCAACTTATTACTGTCAAAAGTATAACAGTGCCCCGTACACTTTTGGCCAAGGGACACGAC
TGGAGATTAAA
SEQ ID NO: 246 (KL2B242 VL cDNA)
TCCTATGAGCTGACTCAGCCACCCTCAGTGTCCGTGTCCCCAGGAGAGACAGCCAGCATCAC
CTGCTCTGGAGATCAATTGGGGGAAAATTATGCTTGCTGGTATCAGCAGAAGCCAGGCCAGT
CCCCTGTGTTGGTCATCTATCAAGATAGTAAGCGGCCCTCAGGGATCCCTGAGCGATTCTCT
GGCTCCAACTCTGGGAACACAGCCACTCTGACCATCAGCGGGACCCAGGCTCTGGATGAGG
CTGACTATTACTGTCAGGCGTGGGACAACAGTATTGTGGTATTCGGCGGAGGGACCAAGCTG
ACCGTCCTA
SEQ ID NO: 247 (KL2B467 VL cDNA)
CAGTCTGTGCTGACTCAGCCACCCTCGGTGTCAGTGGCCCCCGGGCAGACGGCCAGTATTAC
CTGTGGGGGAGACAACATTGGAAGTAAAAGTGTGCACTGGTACCAGCAGAAGCCAGGCCAG
GCCCCTGTGCTGGTCGTCTATGATAATAGCGACCGGCCCTCAGGGATCCCTGAGCGATTCTC
TGGCTCCAACTCTGGGACCACGGCCACCCTGACCATCAGCAGGGTCGAAGCCGGGGATGAG
GCCGACTATTACTGTCAGGTGTGGGATAGTAGTAGTGATCATCCTGTGGTATTCGGCGGAGG
GACCAAGGTCACCGTCCTA
SEQ ID: 271 (KLK2B494_VL DNA)
TCTTCTGAGCTGACTCAGCCACCCTCGGTGTCAGTGGCCCCAGGACAGACGGCCAGGATTAC
CTGTGGGGGAAACAACATTGGAAGTAAAAGTGTGCACTGGTACCAGCAGAAGCCAGGCCAG
GCCCCTGTGCTGGTCGTCTATGATGATAGCGACCGGCCCTCAGGGATCCCTGAGCGATTCTC
TGGCTCCAACTCTGGGAACACGGCCACCCTGACCATCAGCAGGGTCGAAGCCGGGGATGAG
GCCGACTATTACTGTCAGGTGTGGGATAGTAGTAGTGATCATGTGGTATTCGGCGGAGGGAC
CAAGCTGACCGTCCTA

Fab-Fc and scFvs

The hK2 specific VH/VL regions were engineered as VH-CH1-hinge CH2-CH3 and VL-CL and expressed as IgG2 or IgG4 or were engineered as scFvs in either the VH-Linker-VL or VL-linker-VH orientations. The linker that is used in the scFv was the linker of SEQ ID NO: 7 described above. The scFv were used to generate bispecific antibodies as described in Example 7 or to generated CAR as described in Example 11.

Table 10 shows the HC amino acid sequences of selected anti-hK2 antibodies in the mAb format. Table 11 shows the LC amino acid sequences of selected anti-hK2 antibodies in a mAb. Table 12 summaries the HC and LC DNA SEQ ID NOs of selected anti-hK2 antibodies in the mAb format. Table 13 shows the amino acid sequences of selected scFvs in VH-linker-VL or VL-linker-VH orientation.

TABLE 10
Amino acid sequence of the HC (VH-CH1-hinge CH2-CH3) of selected anti-hK2 antibodies in
a mAb format.

| | HC | |
|---|---|---|
| HK2 | PROTEIN | |
| HEAVY | SEQ ID | |
| CHAIN | NO: | HC AMINO ACID SEQUENCE |
| m11B6_HC | 202 | DVQLQESGPGLVKPSQSLSLTCTVTGNSITSDYAWNWIRQFP |
| | | GNRLEWMGYISYSGSTTYSPSLKSRFSITRDTSKNQFFLQLNS |
| | | VTPEDTATYFCATGYYYGSGFWGQGTLVTVSSAKTTAPSVY |
| | | PLAPVCGDTTGSSVTLGCLVKGYFPEPVTLTWNSGSLSSGVH |
| | | TFPAVLQSDLYTLSSSVTVTSSTWPSQSITCNVAHPASSTKVD |
| | | KKIEPRGPTIKPCPPCKCPAPNLLGGPSVFIFPPKIKDVLMISLS |
| | | PIVTCVVVDVSEDDPDVQISWFVNNVEVHTAQTQTHREDYN |
| | | STLRVVSALPIQHQDWMSGKEFKCKVNNKDLPAPIERTISKP |
| | | KGSVRAPQVYVLPPPEEEMTKKQVTLTCMVTDFMPEDIYVE |
| | | WTNNGKTELNYKNTEPVLDSDGSYFMYSKLRVEKKNWVER |
| | | NSYSCSVVHEGLHNHHTTKSFSRTPGK |
| h11B6_HC | 203 | QVQLQESGPGLVKPSDTLSLTCAVSGNSITSDYAWNWIRQPP |
| | | GKGLEWIGYISYSGSTTYNPSLKSRVTMSRDTSKNQFSLKLSS |
| | | VTAVDTAVYYCATGYYYGSGFWGQGTLVTVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNTK |
| | | VDKKVEPKSCDKTHTCPPCPAPELLGGPSVFLFPPKPKDTLMI |
| | | SRTPEVTCVVVDVSHEDPEVKFNWYVDGVEVHNAKTKPRE |
| | | EQYNSTYRVVSVLTVLHQDWLNGKEYKCKVSNKALPAPIEK |
| | | TISKAKGQPREPQVYTLPPSREEMTKNQVSLTCLVKGFYPSDI |
| | | AVEWESNGQPENNYKTTPPVLDSDGSFFLYSKLTVDKSRWQ |
| | | QGNVFSCSVMHEALHNHYTQKSLSLSPGK |
| KL2B30_HC | 210 | QVQLQESGPGLVKPSETLSLTCTVSGGSISSYYWSWIRQPPGK |
| | | GLEWIGYIYYSGSTNYNPSLKSRVTISVDTSKNQFSLKLSSVT |
| | | AADTAVYYCAGTTIFGVVTPNFYYGMDVWGQGTTVTVSSA |
| | | STKGPSVFPLAPCSRSTSESTAALGCLVKDYFPEPVTVSWNSG |
| | | ALTSGVHTFPAVLQSSGLYSLSSVVTVPSSSLGTKTYTCNVD |
| | | HKPSNTKVDKRVESKYGPPCPPCPAPEAAGGPSVFLFPPKPK |
| | | DTLMISRTPEVTCVVVDVSQEDPEVQFNWYVDGVEVHNAK |
| | | TKPREEQFNSTYRVVSVLTVLHQDWLNGKEYKCKVSNKGLP |
| | | SSIEKTISKAKGQPREPQVYTLPPSQEEMTKNQVSLTCLVKGF |
| | | YPSDIAVEWESNGQPENNYKTTPPVLDSDGSFFLYSRLTVDK |
| | | SRWQEGNVFSCSVMHEALHNHYTQKSLSLSLGK |
| K2B53_HC | 211 | EVQLVESGGGVVQPGRSLRLSCVASGFTFSSYDIHWVRQAPG |
| | | KGLEWVAIISYDGSKKDYTDSVKGRFTISRDNSKNTLYLQM |
| | | DSLRVEDSAVYSCARESGWSHYYYYGMDVWGQGTMVTVS |
| | | SASTKGPSVFPLAPCSRSTSESTAALGCLVKDYFPEPVTVSWN |
| | | SGALTSGVHTFPAVLQSSGLYSLSSVVTVPSSSLGTKTYTCNV |
| | | DHKPSNTKVDKRVESKYGPPCPPCPAPEAAGGPSVFLFPPKP |
| | | KDTLMISRTPEVTCVVVDVSQEDPEVQFNWYVDGVEVHNA |
| | | KTKPREEQFNSTYRVVSVLTVLHQDWLNGKEYKCKVSNKG |
| | | LPSSIEKTISKAKGQPREPQVYTLPPSQEEMTKNQVSLTCLVK |
| | | GFYPSDIAVEWESNGQPENNYKTTPPVLDSDGSFFLYSRLTV |
| | | DKSRWQEGNVFSCSVMHEALHNHYTQKSLSLSLGK |
| KL2B242_HC | 212 | QVQLQESGPGLVKPSETLSLTCTVSGGSISSYYWSWLRQPAG |
| | | SGLEWIGRLYVSGFTNYNPSLKSRVTLSLDPSRNQLSLKLSSV |
| | | TAADTAVYYCAGDSGNYWGWFDPWGQGTLVTVSSASTKG |
| | | PSVFPLAPCSRSTSESTAALGCLVKDYFPEPVTVSWNSGALTS |
| | | GVHTFPAVLQSSGLYSLSSVVTVPSSSLGTKTYTCNVDHKPS |
| | | NTKVDKRVESKYGPPCPPCPAPEAAGGPSVFLFPPKPKDTLM |
| | | ISRTPEVTCVVVDVSQEDPEVQFNWYVDGVEVHNAKTKPRE |
| | | EQFNSTYRVVSVLTVLHQDWLNGKEYKCKVSNKGLPSSIEK |
| | | TISKAKGQPREPQVYTLPPSQEEMTKNQVSLTCLVKGFYPSDI |
| | | AVEWESNGQPENNYKTTPPVLDSDGSFFLYSRLTVDKSRWQ |
| | | EGNVFSCSVMHEALHNHYTQKSLSLSLGK |
| KL2B467_HC | 213 | QVQLVESGGGVVQPGRSLRLSCAASGFTFSYYGMHWVRQA |
| | | PGKGLEWVAFISYDGSNKYYADSVKGRFTISRDNSKNTLYL |
| | | QMNSLRAEDTAVYYCAHLPYSGSYWAFDYWGQGTQVTVSS |
| | | ASTKGPSVFPLAPSSKSTSGGTAALGCLVKDYFPEPVTVSWN |
| | | SGALTSGVHTFPAVLQSSGLYSLSSVVTVPSSSLGTQTYICNV |
| | | NHKPSNTKVDKKVEPKSCDKTHTCPPCPAPEAAGGPSVFLFP |
| | | PKPKDTLMISRTPEVTCVVVSVSHEDPEVKFNWYVDGVEVH |
| | | NAKTKPREEQYNSTYRVVSVLTVLHQDWLNGKEYKCKVSN |
| | | KALPAPIEKTISKAKGQPREPQVYTLPPSREEMTKNQVSLTCL |
| | | VKGFYPSDIAVEWESNGQPENNYKTTPPVLDSDGSFFLYSKL |
| | | TVDKSRWQQGNVFSCSVMHEALHNHYTQKSLSLSPGK |
| KL2B494_HC | 219 | QVQLVESGGGLVQPGGSLRLSCAASGFTFSHYAMSWVRQAP |
| | | GKGLEWVSTIGGSGGSTYYADSVKGRFTISRDNSKNTLYLQ |
| | | MNSLRAEDTAVYYCAKPHIVMVTALLYDGMDVWGQGTMV |
| | | TVSSASTKGPSVFPLAPSSKSTSGGTAALGCLVKDYFPEPVTV |
| | | SWNSGALTSGVHTFPAVLQSSGLYSLSSVVTVPSSSLGTQTYI |
| | | CNVNHKPSNTKVDKKVEPKSCDKTHTCPPCPAPEAAGGPSV |
| | | FLFPPKPKDTLMISRTPEVTCVVVSVSHEDPEVKFNWYVDGV |
| | | EVHNAKTKPREEQYNSTYRVVSVLTVLHQDWLNGKEYKCK |
| | | VSNKALPAPIEKTISKAKGQPREPQVYTLPPSREEMTKNQVSL |
| | | TCLVKGFYPSDIAVEWESNGQPENNYKTTPPVLDSDGSFFLY |
| | | SKLTVDKSRWQQGNVFSCSVMHEALHNHYTQKSLSLSPGK |

TABLE 11
Amino acid sequences of the LC (VL-CL) of selected anti-hK2 antibodies in a mAb (Fab-Fc)
format.

| HK2 | LC | |
|---|---|---|
| LIGHT | PROTEIN | |
| CHAIN | SEQ ID NO: | LC AMINO ACID SEQUENCE |
| m11B6_LC | 214 | DIVLTQSPASLAVSLGQRATISCRASESVEYFGTSLMHWYRQKPGQP |
| | | PKLLIYAASNVESGVPARFSGSGSGTDFSLNIQPVEEDDFSMYFCQQ |
| | | TRKVPYTFGGGTKLEIKRADAAPTVSIFPPSSEQLTSGGASVVCFLN |
| | | NFYPKDINVKWKIDGSERQNGVLNSWTDQDSKDSTYSMSSTLTLTK |
| | | DEYERHNSYTCEATHKTSTSPIVKSFNRNEC |
| h11B6_LC | 215 | DIVLTQSPDSLAVSLGERATINCKASESVEYFGTSLMHWYQQKPGQ |
| | | PPKLLIYAASNRESGVPDRFSGSGSGTDFTLTISSLQAEDVAVYYCQ |
| | | QTRKVPYTFGQGTKLEIKRTVAAPSVFIFPPSDEQLKSGTASVVCLL |
| | | NNFYPREAKVQWKVDNALQSGNSQESVTEQDSKDSTYSLSSTLTLS |
| | | KADYEKHKVYACEVTHQGLSSPVTKSFNRGEC |
| KL2B30_LC | 221 | DIQMTQSPSFLSASVGDRVTITCRASQGISSYLAWYQQKPGKAPKFL |
| | | IYAASTLQSGVPSRFSGSGSGTEFTLTISSLQPEDFATYYCQQLNSYP |
| | | LTFGGGTKVEIKRTVAAPSVFIFPPSDEQLKSGTASVVCLLNNFYPRE |
| | | AKVQWKVDNALQSGNSQESVTEQDSKDSTYSLSSTLTLSKADYEK |
| | | HKVYACEVTHQGLSSPVTKSFNRGEC |
| KL2B53 LC | 222 | DIVMTQSPSSLSASVGDRVTITCRASQDISNYLAWYQQKPGKVPKFL |
| | | IYAASTLHSGVPSRFSGSGSGTDFTLTISSLQPEDVATYYCQKYNSAP |
| | | YTFGQGTRLEIKRTVAAPSVFIFPPSDEQLKSGTASVVCLLNNFYPRE |
| | | AKVQWKVDNALQSGNSQESVTEQDSKDSTYSLSSTLTLSKADYEK |
| | | HKVYACEVTHQGLSSPVTKSFNRGEC |
| KL2B242_LC | 223 | SYELTQPPSVSVSPGETASITCSGDQLGENYACWYQQKPGQSPVLVI |
| | | YQDSKRPSGIPERFSGSNSGNTATLTISGTQALDEADYYCQAWDNSI |
| | | VVFGGGTKLTVLGQPKAAPSVTLFPPSSEELQANKATLVCLISDFYP |
| | | GAVTVAWKADSSPVKAGVETTTPSKQSNNKYAASSYLSLTPEQWK |
| | | SHRSYSCQVTHEGSTVEKTVAPTECS |
| KL2B467_LC | 224 | QSVLTQPPSVSVAPGQTASITCGGDNIGSKSVHWYQQKPGQAPVLV |
| | | VYDNSDRPSGIPERFSGSNSGTTATLTISRVEAGDEADYYCQVWDSS |
| | | SDHPVVFGGGTKVTVLGQPKAAPSVTLFPPSSEELQANKATLVCLIS |
| | | DFYPGAVTVAWKADSSPVKAGVETTTPSKQSNNKYAASSYLSLTPE |
| | | QWKSHRSYSCQVTHEGSTVEKTVAPTECS |
| KL2B494_LC | 220 | SSELTQPPSVSVAPGQTARITCGGNNIGSKSVHWYQQKPGQAPVLV |
| | | VYDDSDRPSGIPERFSGSNSGNTATLTISRVEAGDEADYYCQVWDS |
| | | SSDHVVFGGGTKLTVLGQPKAAPSVTLFPPSSEELQANKATLVCLIS |
| | | DFYPGAVTVAWKADSSPVKAGVETTTPSKQSNNKYAASSYLSLTPE |
| | | QWKSHRSYSCQVTHEGSTVEKTVAPTECS |

TABLE 12
SEQ ID Nos of the cDNA sequences of
HC and LC of selected hK2 antibodies

| | HC | LC | HC | LC |
|---|---|---|---|---|
| | Protein | Protein | cDNA | cDNA |
| | SEQ ID | SEQ ID | SEQ ID | SEQ ID |
| Antibody | NO: | NO: | NO: | NO: |
| m11B6 | 202 | 214 | 284 | 296 |
| hu11B6 | 203 | 215 | 285 | 297 |
| KL2B30 | 210 | 221 | 292 | 450 |
| KL2B53 | 211 | 222 | 293 | 451 |
| KL2B242 | 212 | 223 | 294 | 305 |
| KL2B467 | 213 | 224 | 295 | 306 |
| KL2B494 | 219 | 220 | 274 | 282 |

TABLE 13
Amino acid sequences of the variable domain of selected anti-hK2 scFvs antibodies in VH-
linker-VL (HL) or in VL-linker-VH (LH) format.

| | | | SEQ |
|---|---|---|---|
| scFv | | | ID |
| name | Acronym | Amino acid sequence of scFv | NO: |
| scFv1 | HCG5_LDC6_HL | QVQLQESGPGLVKPSDTLSLTCAVSGNSITSDYAWN | 8 |
| | | WIRQFPGKGLEWMGYISYSGSTTYNPSLKSRVTISRD | |
| | | TSKNQFSLKLSSVTPVDTAVYYCATGYYYGSGFWG | |
| | | QGTLVTVSSGGSEGKSSGSGSESKSTGGSDIVLTQSP | |
| | | DSLAVSLGERATINCKASESVEYFGTSLMHWYQQKP | |
| | | GQPPKLLIYAASNRESGVPDRFSGSGSGTDFTLTIQSV | |
| | | QAEDVSVYFCQQTRKVPYTFGQGTKLEIK | |
| scFv2 | HCG5_hu11B6_HL | QVQLQESGPGLVKPSDTLSLTCAVSGNSITSDYAWN | 9 |
| | | WIRQFPGKGLEWMGYISYSGSTTYNPSLKSRVTISRD | |
| | | TSKNQFSLKLSSVTPVDTAVYYCATGYYYGSGFWG | |
| | | QGTLVTVSSGGSEGKSSGSGSESKSTGGSDIVLTQSP | |
| | | DSLAVSLGERATINCKASESVEYFGTSLMHWYQQKP | |
| | | GQPPKLLIYAASNRESGVPDRFSGSGSGTDFTLTISSL | |
| | | QAEDVAVYYCQQTRKVPYTFGQGTKLEIK | |
| scFv3 | HCF3_hu11B6_HL | QVQLQESGPGLVKPSDTLSLTCAVSGNSITSDYAWN | 10 |
| | | WIRQFPGKGLEWIGYISYSGSTTYNPSLKSRVTISRDT | |
| | | SKNQFSLKLSSVTPVDTAVYYCATGYYYGSGFWGQ | |
| | | GTLVTVSSGGSEGKSSGSGSESKSTGGSDIVLTQSPD | |
| | | SLAVSLGERATINCKASESVEYFGTSLMHWYQQKPG | |
| | | QPPKLLIYAASNRESGVPDRFSGSGSGTDFTLTISSVQ | |
| | | AEDVAVYYCQQTRKVPYTFGQGTKLEIK | |
| scFv4 | HCG5_LCB7_HL | QVQLQESGPGLVKPSDTLSLTCAVSGNSITSDYAWN | 11 |
| | | WIRQFPGKGLEWMGYISYSGSTTYNPSLKSRVTISRD | |
| | | TSKNQFSLKLSSVTPVDTAVYYCATGYYYGSGFWG | |
| | | QGTLVTVSSGGSEGKSSGSGSESKSTGGSDIVLTQSP | |
| | | DSLAVSLGERATINCKASESVEYFGTSLMHWYQQKP | |
| | | GQPPKLLIYAASNRESGVPDRFSGSGSGTDFTLTISSV | |
| | | QAEDVAVYYCQQTRKVPYTFGQGTKLEIK | |
| scFv5 | LCD6_HCG5_LH | DIVLTQSPDSLAVSLGERATINCKASESVEYFGTSLM | 12 |
| | | HWYQQKPGQPPKLLIYAASNRESGVPDRFSGSGSGT | |
| | | DFTLTIQSVQAEDVSVYFCQQTRKVPYTFGQGTKLEI | |
| | | KGGSEGKSSGSGSESKSTGGSQVQLQESGPGLVKPS | |
| | | DTLSLTCAVSGNSITSDYAWNWIRQFPGKGLEWMG | |
| | | YISYSGSTTYNPSLKSRVTISRDTSKNQFSLKLSSVTP | |
| | | VDTAVYYCATGYYYGSGFWGQGTLVTVSS | |
| scFv6 | hu11B6_HCF3_LH | DIVLTQSPDSLAVSLGERATINCKASESVEYFGTSLM | 13 |
| | | HWYQQKPGQPPKLLIYAASNRESGVPDRFSGSGSGT | |
| | | DFTLTISSLQAEDVAVYYCQQTRKVPYTFGQGTKLEI | |
| | | KGGSEGKSSGSGSESKSTGGSQVQLQESGPGLVKPS | |
| | | DTLSLTCAVSGNSITSDYAWNWIRQFPGKGLEWIGYI | |
| | | SYSGSTTYNPSLKSRVTISRDTSKNQFSLKLSSVTPVD | |
| | | TAVYYCATGYYYGSGFWGQGTLVTVSS | |
| scFv7 | hu11B6_HCG5_LH | DIVLTQSPDSLAVSLGERATINCKASESVEYFGTSLM | 14 |
| | | HWYQQKPGQPPKLLIYAASNRESGVPDRFSGSGSGT | |
| | | DFTLTISSLQAEDVAVYYCQQTRKVPYTFGQGTKLEI | |
| | | KGGSEGKSSGSGSESKSTGGSQVQLQESGPGLVKPS | |
| | | DTLSLTCAVSGNSITSDYAWNWIRQFPGKGLEWMG | |
| | | YISYSGSTTYNPSLKSRVTISRDTSKNQFSLKLSSVTP | |
| | | VDTAVYYCATGYYYGSGFWGQGTLVTVSS | |
| scFv8 | LCB7_HCF3_LH | DIVLTQSPDSLAVSLGERATINCKASESVEYFGTSLM | 15 |
| | | HWYQQKPGQPPKLLIYAASNRESGVPDRFSGSGSGT | |
| | | DFTLTISSVQAEDVAVYYCQQTRKVPYTFGQGTKLE | |
| | | IKGGSEGKSSGSGSESKSTGGSQVQLQESGPGLVKPS | |
| | | DTLSLTCAVSGNSITSDYAWNWIRQFPGKGLEWIGYI | |
| | | SYSGSTTYNPSLKSRVTISRDTSKNQFSLKLSSVTPVD | |
| | | TAVYYCATGYYYGSGFWGQGTLVTVSS | |
| scFv9 | LCB7_HCG5_LH | DIVLTQSPDSLAVSLGERATINCKASESVEYFGTSLM | 16 |
| | | HWYQQKPGQPPKLLIYAASNRESGVPDRFSGSGSGT | |
| | | DFTLTISSVQAEDVAVYYCQQTRKVPYTFGQGTKLE | |
| | | IKGGSEGKSSGSGSESKSTGGSQVQLQESGPGLVKPS | |
| | | DTLSLTCAVSGNSITSDYAWNWIRQFPGKGLEWMG | |
| | | YISYSGSTTYNPSLKSRVTISRDTSKNQFSLKLSSVTP | |
| | | VDTAVYYCATGYYYGSGFWGQGTLVTVSS | |
| scFv10 | LCD6_HCF3_LH | DIVLTQSPDSLAVSLGERATINCKASESVEYFGTSLM | 17 |
| | | HWYQQKPGQPPKLLIYAASNRESGVPDRFSGSGSGT | |
| | | DFTLTIQSVQAEDVSVYFCQQTRKVPYTFGQGTKLEI | |
| | | KGGSEGKSSGSGSESKSTGGSQVQLQESGPGLVKPS | |
| | | DTLSLTCAVSGNSITSDYAWNWIRQFPGKGLEWIGYI | |
| | | SYSGSTTYNPSLKSRVTISRDTSKNQFSLKLSSVTPVD | |
| | | TAVYYCATGYYYGSGFWGQGTLVTVSS | |
| scFv11 | hu11B6_LCB7_HL | QVQLQESGPGLVKPSDTLSLTCAVSGNSITSDYAWN | 18 |
| | | WIRQPPGKGLEWIGYISYSGSTTYNPSLKSRVTMSRD | |
| | | TSKNQFSLKLSSVTAVDTAVYYCATGYYYGSGFWG | |
| | | QGTLVTVSSGGSEGKSSGSGSESKSTGGSDIVLTQSP | |
| | | DSLAVSLGERATINCKASESVEYFGTSLMHWYQQKP | |
| | | GQPPKLLIYAASNRESGVPDRFSGSGSGTDFTLTISSV | |
| | | QAEDVAVYYCQQTRKVPYTFGQGTKLEIK | |
| scFv12 | hu11B6_LCD6_HL | QVQLQESGPGLVKPSDTLSLTCAVSGNSITSDYAWN | 19 |
| | | WIRQPPGKGLEWIGYISYSGSTTYNPSLKSRVTMSRD | |
| | | TSKNQFSLKLSSVTAVDTAVYYCATGYYYGSGFWG | |
| | | QGTLVTVSSGGSEGKSSGSGSESKSTGGSDIVLTQSP | |
| | | DSLAVSLGERATINCKASESVEYFGTSLMHWYQQKP | |
| | | GQPPKLLIYAASNRESGVPDRFSGSGSGTDFTLTIQSV | |
| | | QAEDVSVYFCQQTRKVPYTFGQGTKLEIK | |
| scFv13 | hu11B6_HL | QVQLQESGPGLVKPSDTLSLTCAVSGNSITSDYAWN | 20 |
| | | WIRQPPGKGLEWIGYISYSGSTTYNPSLKSRVTMSRD | |
| | | TSKNQFSLKLSSVTAVDTAVYYCATGYYYGSGFWG | |
| | | QGTLVTVSSGGSEGKSSGSGSESKSTGGSDIVLTQSP | |
| | | DSLAVSLGERATINCKASESVEYFGTSLMHWYQQKP | |
| | | GQPPKLLIYAASNRESGVPDRFSGSGSGTDFTLTISSL | |
| | | QAEDVAVYYCQQTRKVPYTFGQGTKLEIK | |
| scFv14 | LCD6_hu11B6_LH | DIVLTQSPDSLAVSLGERATINCKASESVEYFGTSLM | 21 |
| | | HWYQQKPGQPPKLLIYAASNRESGVPDRFSGSGSGT | |
| | | DFTLTIQSVQAEDVSVYFCQQTRKVPYTFGQGTKLEI | |
| | | KGGSEGKSSGSGSESKSTGGSQVQLQESGPGLVKPS | |
| | | DTLSLTCAVSGNSITSDYAWNWIRQPPGKGLEWIGYI | |
| | | SYSGSTTYNPSLKSRVTMSRDTSKNQFSLKLSSVTAV | |
| | | DTAVYYCATGYYYGSGFWGQGTLVTVSS | |
| scFv15 | hu11B6_LH | DIVLTQSPDSLAVSLGERATINCKASESVEYFGTSLM | 22 |
| | | HWYQQKPGQPPKLLIYAASNRESGVPDRFSGSGSGT | |
| | | DFTLTISSLQAEDVAVYYCQQTRKVPYTFGQGTKLEI | |
| | | KGGSEGKSSGSGSESKSTGGSQVQLQESGPGLVKPS | |
| | | DTLSLTCAVSGNSITSDYAWNWIRQPPGKGLEWIGYI | |
| | | SYSGSTTYNPSLKSRVTMSRDTSKNQFSLKLSSVTAV | |
| | | DTAVYYCATGYYYGSGFWGQGTLVTVSS | |
| scFv16 | LCB7_hu11B6_LH | DIVLTQSPDSLAVSLGERATINCKASESVEYFGTSLM | 23 |
| | | HWYQQKPGQPPKLLIYAASNRESGVPDRFSGSGSGT | |
| | | DFTLTISSVQAEDVAVYYCQQTRKVPYTFGQGTKLE | |
| | | IKGGSEGKSSGSGSESKSTGGSQVQLQESGPGLVKPS | |
| | | DTLSLTCAVSGNSITSDYAWNWIRQPPGKGLEWIGYI | |
| | | SYSGSTTYNPSLKSRVTMSRDTSKNQFSLKLSSVTAV | |
| | | DTAVYYCATGYYYGSGFWGQGTLVTVSS | |
| scFv17 | KL2B413_HL | EVQLVESGGGLVQPGGSLRLSCAASGFTFSSYWMT | 133 |
| | | WVRQAPGKGLEWVANIKQDGSERYYVDSVKGRFTI | |
| | | SRDNAKNSLYLQMNSLRAEDTAVYYCARDQNYDIL | |
| | | TGHYGMDVWGQGTTVTVSSGGSEGKSSGSGSESKS | |
| | | TGGSEIVLTQSPSFLSASVGDRVTITCRASQGISSYLS | |
| | | WYQQKPGKAPKLLIYATSTLQSGVPSRFSGSGSGTEF | |
| | | TLTISSLQPEDFATYYCQQLNSYPRTFGQGTKVEIK | |
| scFv18 | KL2B413_LH | EIVLTQSPSFLSASVGDRVTITCRASQGISSYLSWYQQ | 134 |
| | | KPGKAPKLLIYATSTLQSGVPSRFSGSGSGTEFTLTIS | |
| | | SLQPEDFATYYCQQLNSYPRTFGQGTKVEIKGGSEG | |
| | | KSSGSGSESKSTGGSEVQLVESGGGLVQPGGSLRLSC | |
| | | AASGFTFSSYWMTWVRQAPGKGLEWVANIKQDGSE | |
| | | RYYVDSVKGRFTISRDNAKNSLYLQMNSLRAEDTA | |
| | | VYYCARDQNYDILTGHYGMDVWGQGTTVTVSS | |
| scFv19 | KL2B359_HL | QVQLQESGPGLVKPSQTLSLTCTVSGNSITSDYAWN | 135 |
| | | WIRQFPGKRLEWIGYISYSGSTTYNPSLKSRVTISRDT | |
| | | SKNQFSLKLSSVTAADTAVYYCATGYYYGSGFWGQ | |
| | | GTLVTVSSGGSEGKSSGSGSESKSTGGSEIVLTQSPAT | |
| | | LSLSPGERATLSCRASESVEYFGTSLMHWYQQKPGQ | |
| | | PPRLLIYAASNVESGIPARFSGSGSGTDFTLTISSVEPE | |
| | | DFAVYFCQQTRKVPYTFGGGTKVEIK | |
| scFv20 | KL2B359_LH | EIVLTQSPATLSLSPGERATLSCRASESVEYFGTSLMH | 136 |
| | | WYQQKPGQPPRLLIYAASNVESGIPARFSGSGSGTDF | |
| | | TLTISSVEPEDFAVYFCQQTRKVPYTFGGGTKVEIKG | |
| | | GSEGKSSGSGSESKSTGGSQVQLQESGPGLVKPSQTL | |
| | | SLTCTVSGNSITSDYAWNWIRQFPGKRLEWIGYISYS | |
| | | GSTTYNPSLKSRVTISRDTSKNQFSLKLSSVTAADTA | |
| | | VYYCATGYYYGSGFWGQGTLVTVSS | |
| scFv21 | KL2B357_HL | QVQLQESGPGLVKPSQTLSLTCTVSGNSITSDYAWN | 318 |
| | | WIRQFPGKGLEWIGYISYSGSTTYNPSLKSRVTISRDT | |
| | | SKNQFSLKLSSVTAADTAVYYCATGYYYGSGFWGQ | |
| | | GTLVTVSSGGSEGKSSGSGSESKSTGGSDIVLTQSPD | |
| | | SLAVSLGERATINCRASESVEYFGTSLMHWYQQKPG | |
| | | QPPKLLIYAASNVESGVPDRFSGSGSGTDFTLTISSLQ | |
| | | AEDVAVYFCQQTRKVPYTFGGGTKVEIK | |
| scFv22 | KL2B357_LH | DIVLTQSPDSLAVSLGERATINCRASESVEYFGTSLM | 319 |
| | | HWYQQKPGQPPKLLIYAASNVESGVPDRFSGSGSGT | |
| | | DFTLTISSLQAEDVAVYFCQQTRKVPYTFGGGTKVEI | |
| | | KGGSEGKSSGSGSESKSTGGSQVQLQESGPGLVKPS | |
| | | QTLSLTCTVSGNSITSDYAWNWIRQFPGKGLEWIGYI | |
| | | SYSGSTTYNPSLKSRVTISRDTSKNQFSLKLSSVTAA | |
| | | DTAVYYCATGYYYGSGFWGQGTLVTVSS | |
| scFv23 | KL2B358_HL | QVQLQESGPGLVKPSQTLSLTCTVSGNSITSDYAWN | 320 |
| | | WIRQPPGKGLEWIGYISYSGSTTYNPSLKSRVTISRDT | |
| | | SKNQFSLKLSSVTAADTAVYYCATGYYYGSGFWGQ | |
| | | GTLVTVSSGGSEGKSSGSGSESKSTGGSEIVLTQSPAT | |
| | | LSLSPGERATLSCRASESVEYFGTSLMHWYQQKPGQ | |
| | | PPRLLIYAASNVESGIPARFSGSGSGTDFTLTISSVEPE | |
| | | DFAVYFCQQTRKVPYTFGGGTKVEIK | |
| scFv24 | KL2B358_LH | EIVLTQSPATLSLSPGERATLSCRASESVEYFGTSLMH | 321 |
| | | WYQQKPGQPPRLLIYAASNVESGIPARFSGSGSGTDF | |
| | | TLTISSVEPEDFAVYFCQQTRKVPYTFGGGTKVEIKG | |
| | | GSEGKSSGSGSESKSTGGSQVQLQESGPGLVKPSQTL | |
| | | SLTCTVSGNSITSDYAWNWIRQPPGKGLEWIGYISYS | |
| | | GSTTYNPSLKSRVTISRDTSKNQFSLKLSSVTAADTA | |
| | | VYYCATGYYYGSGFWGQGTLVTVSS | |
| scFv25 | KL2B360_HL | QVQLQESGPGLVKPSQTLSLTCTVSGNSITSDYAWN | 322 |
| | | WIRQFPGKGLEWIGYISYSGSTTYNPSLKSRVTISRDT | |
| | | SKNQFSLKLSSVTAADTAVYYCATGYYYGSGFWGQ | |
| | | GTLVTVSSGGSEGKSSGSGSESKSTGGSEIVLTQSPAT | |
| | | LSLSPGERATLSCRASESVEYFGTSLMHWYQQKPGQ | |
| | | PPRLLIYAASNVESGIPARFSGSGSGTDFTLTISSVEPE | |
| | | DFAVYFCQQTRKVPYTFGGGTKVEIK | |
| scFv26 | KL2B360_LH | EIVLTQSPATLSLSPGERATLSCRASESVEYFGTSLMH | 323 |
| | | WYQQKPGQPPRLLIYAASNVESGIPARFSGSGSGTDF | |
| | | TLTISSVEPEDFAVYFCQQTRKVPYTFGGGTKVEIKG | |
| | | GSEGKSSGSGSESKSTGGSQVQLQESGPGLVKPSQTL | |
| | | SLTCTVSGNSITSDYAWNWIRQFPGKGLEWIGYISYS | |
| | | GSTTYNPSLKSRVTISRDTSKNQFSLKLSSVTAADTA | |
| | | VYYCATGYYYGSGFWGQGTLVTVSS | |
| scFv27 | KL2B467_HL | QVQLVESGGGVVQPGRSLRLSCAASGFTFSYYGMH | 324 |
| | | WVRQAPGKGLEWVAFISYDGSNKYYADSVKGRFTI | |
| | | SRDNSKNTLYLQMNSLRAEDTAVYYCAHLPYSGSY | |
| | | WAFDYWGQGTQVTVSSGGSEGKSSGSGSESKSTGG | |
| | | SQSVLTQPPSVSVAPGQTASITCGGDNIGSKSVHWY | |
| | | QQKPGQAPVLVVYDNSDRPSGIPERFSGSNSGTTATL | |
| | | TISRVEAGDEADYYCQVWDSSSDHPVVFGGGTKVT | |
| | | V | |
| scFv28 | KL2B467_LH | QSVLTQPPSVSVAPGQTASITCGGDNIGSKSVHWYQ | 325 |
| | | QKPGQAPVLVVYDNSDRPSGIPERFSGSNSGTTATLT | |
| | | ISRVEAGDEADYYCQVWDSSSDHPVVFGGGTKVTV | |
| | | GGSEGKSSGSGSESKSTGGSQVQLVESGGGVVQPGR | |
| | | SLRLSCAASGFTFSYYGMHWVRQAPGKGLEWVAFI | |
| | | SYDGSNKYYADSVKGRFTISRDNSKNTLYLQMNSLR | |
| | | AEDTAVYYCAHLPYSGSYWAFDYWGQGTQVTVSS | |
| scFv39 | KL2B494_HL | QVQLVESGGGLVQPGGSLRLSCAASGFTFSHYAMS | 308 |
| | | WVRQAPGKGLEWVSTIGGSGGSTYYADSVKGRFTIS | |
| | | RDNSKNTLYLQMNSLRAEDTAVYYCAKPHIVMVTA | |
| | | LLYDGMDVWGQGTMVTVSS | |
| | | GGSEGKSSGSGSESKSTGGSSSELTQPPSVSVAPGQT | |
| | | ARITCGGNNIGSKSVHWYQQKPGQAPVLVVYDDSD | |
| | | RPSGIPERFSGSNSGNTATLTISRVEAGDEADYYCQV | |
| | | WDSSSDHVVFGGGTKLTVL | |
| scFv40 | KL2B494_LH | SSELTQPPSVSVAPGQTARITCGGNNIGSKSVHWYQ | 316 |
| | | QKPGQAPVLVVYDDSDRPSGIPERFSGSNSGNTATLT | |
| | | ISRVEAGDEADYYCQVWDSSSDHVVFGGGTKLTVL | |
| | | GGSEGKSSGSGSESKSTGGSQVQLVESGGGLVQPGG | |
| | | SLRLSCAASGFTFSHYAMSWVRQAPGKGLEWVSTIG | |
| | | GSGGSTYYADSVKGRFTISRDNSKNTLYLQMNSLRA | |
| | | EDTAVYYCAKPHIVMVTALLYDGMDVWGQGTMVT | |
| | | VSS | |
| scFv41 | KL2B30_HL | QVQLQESGPGLVKPSETLSLTCTVSGGSISSYYWSWI | 404 |
| | | RQPPGKGLEWIGYIYYSGSTNYNPSLKSRVTISVDTS | |
| | | KNQFSLKLSSVTAADTAVYYCAGTTIFGVVTPNFYY | |
| | | GMDVWGQGTTVTVSSGGSEGKSSGSGSESKSTGGS | |
| | | DIQMTQSPSFLSASVGDRVTITCRASQGISSYLAWYQ | |
| | | QKPGKAPKFLIYAASTLQSGVPSRFSGSGSGTEFTLTI | |
| | | SSLQPEDFATYYCQQLNSYPLTFGGGTKVEIK | |
| scFv42 | KL2B30_LH | DIQMTQSPSFLSASVGDRVTITCRASQGISSYLAWYQ | 405 |
| | | QKPGKAPKFLIYAASTLQSGVPSRFSGSGSGTEFTLTI | |
| | | SSLQPEDFATYYCQQLNSYPLTFGGGTKVEIKGGSEG | |
| | | KSSGSGSESKSTGGSQVQLQESGPGLVKPSETLSLTC | |
| | | TVSGGSISSYYWSWIRQPPGKGLEWIGYIYYSGSTNY | |
| | | NPSLKSRVTISVDTSKNQFSLKLSSVTAADTAVYYCA | |
| | | GTTIFGVVTPNFYYGMDVWGQGTTVTVSS | |
| scFv43 | KL2B53_HL | EVQLVESGGGVVQPGRSLRLSCVASGFTFSSYDIHW | 406 |
| | | VRQAPGKGLEWVAIISYDGSKKDYTDSVKGRFTISR | |
| | | DNSKNTLYLQMDSLRVEDSAVYSCARESGWSHYYY | |
| | | YGMDVWGQGTMVTVSSGGSEGKSSGSGSESKSTGG | |
| | | SDIVMTQSPSSLSASVGDRVTITCRASQDISNYLAWY | |
| | | QQKPGKVPKFLIYAASTLHSGVPSRFSGSGSGTDFTL | |
| | | TISSLQPEDVATYYCQKYNSAPYTFGQGTRLEIK | |
| scFv44 | KL2B53_LH | DIVMTQSPSSLSASVGDRVTITCRASQDISNYLAWYQ | 407 |
| | | QKPGKVPKFLIYAASTLHSGVPSRFSGSGSGTDFTLTI | |
| | | SSLQPEDVATYYCQKYNSAPYTFGQGTRLEIKGGSE | |
| | | GKSSGSGSESKSTGGSEVQLVESGGGVVQPGRSLRL | |
| | | SCVASGFTFSSYDIHWVRQAPGKGLEWVAIISYDGS | |
| | | KKDYTDSVKGRFTISRDNSKNTLYLQMDSLRVEDSA | |
| | | VYSCARESGWSHYYYYGMDVWGQGTMVTVSS | |
| scFv45 | KL2B242_HL | QVQLQESGPGLVKPSETLSLTCTVSGGSISSYYWSWL | 408 |
| | | RQPAGSGLEWIGRLYVSGFTNYNPSLKSRVTLSLDPS | |
| | | RNQLSLKLSSVTAADTAVYYCAGDSGNYWGWFDP | |
| | | WGQGTLVTVSSGGSEGKSSGSGSESKSTGGSSYELT | |
| | | QPPSVSVSPGETASITCSGDQLGENYACWYQQKPGQ | |
| | | SPVLVIYQDSKRPSGIPERFSGSNSGNTATLTISGTQA | |
| | | LDEADYYCQAWDNSIVVFGGGTKLTVL | |
| scFv46 | KL2B242_LH | SYELTQPPSVSVSPGETASITCSGDQLGENYACWYQ | 409 |
| | | QKPGQSPVLVIYQDSKRPSGIPERFSGSNSGNTATLTI | |
| | | SGTQALDEADYYCQAWDNSIVVFGGGTKLTVLGGS | |
| | | EGKSSGSGSESKSTGGSQVQLQESGPGLVKPSETLSL | |
| | | TCTVSGGSISSYYWSWLRQPAGSGLEWIGRLYVSGF | |
| | | TNYNPSLKSRVTLSLDPSRNQLSLKLSSVTAADTAV | |
| | | YYCAGDSGNYWGWFDPWGQGTLVTVSS | |

### Example 24. Biophysical Characterization of Anti-hK2 Antibodies

### Affinity and Thermal Stability of Anti-hK2 Antibodies.

Affinity of selected hK2 antibodies for soluble hK2 was measured by surface plasmon resonance (SPR). SPR is a label-free technique to study the strength of an interaction between two binding partners by measuring the change in mass upon complex formation and dissociation. Antibodies were captured on a sensor chip coated with an anti-Fc antibody followed by injection of soluble hK2 at various concentrations and specified association and dissociation times. Post dissociation, the surface was regenerated with an appropriate solution to prepare for the next interaction. Kinetic information (on-rate and off-rate constants) were extracted by fitting sensorgrams to the 1:1 Langmuir model. Binding affinity (K_{D}) are reported as the ratio of rate constants (k_{off}/kₒₙ). K_{D} values of selected hK2 antibodies are listed in Table 14.

Thermal stability was determined by Differential Scanning Fluorimetry (NanoDSF) using an automated Prometheus instrument. NanoDSF was used to measure Tₘ of molecules at a concentration of 0.5 mg/mL in Phosphate Buffered Saline, pH 7.4. Measurements were made by loading samples into 24 well capillary from a 384 well sample plate. Duplicate runs were performed for each sample. The thermal scans span from 20° C. to 95° C. at a rate of 1.0° C./minute. Intrinsic tryptophan and tyrosine fluorescence were monitored at the emission wavelengths of 330 nm and 350 nm, and the F350/F330 nm ratio were plotted against temperature to generate unfolding curves. Measured Tₘ values are listed in Table 14.

TABLE 14
K_{D} and Tm of selected molecules

| Molecule | K_{D} (nM) | Tm (° C.) |
|---|---|---|
| KL2B413 (scFv-LH-Fc) | 34.3 | 67 |
| KL2B359 (scFv-LH-Fc) | 0.7-1 | 67 |
| KL2B30 (Fab) | 0.460 | >70 |
| KL2B242 (Fab) | 0.040 | >70 |
| KL2B53 (Fab) | 0.080 | >70 |
| KL2B467 (Fab) | 0.078 | >70 |
| KL2B494 (Fab) | 0.053 | >70 |

KL2B413 scFv generated from the Ablexis immunization campaign had a thermal stability (Tₘ) of 67° C. as measured by Nano DSF and a binding affinity (K_{D}) to human hK2 of about 34 nM. Clone KL2B359 obtained for the re-humanization campaign and which had maintained a binding affinity similar to murine 11B6 was converted to scFv-Fc and CAR-T for additional profiling. KL2B359 scFv shows a Tₘ of 67° C. and a binding affinity (K_{D}) to hK2 of ˜0.7-1 nM. KL2B30, KL2B242, KL2B53, KL2B467 and KL2B494 Fab showed binding affinities below 0.5 nM and Tₘ values above 70° C.

### Epitope Mapping

The epitope and paratope of selected anti-hK2 antibodies and anti-hK2/CD3 bispecific antibodies were determined by hydrogen-deuterium exchange mass spectrometry (HDX-MS). Human KLK2 antigen was used for epitope and paratope mapping experiment.

Briefly, purified KLK2 antigen was incubated with and without anti-hK2 antibodies or anti-hK2/CD3 bispecific antibodies in deuterium oxide labeling buffer. The hydrogen-deuterium exchange (HDX) mixture was quenched at different time point by the addition of 8 M urea, 1M TCEP, pH 3.0. The quenched sample was passed over an immobilized pepsin/FPXIII column at 600 μL/min equilibrated with buffer A (1% acetonitrile, 0.1% FA in H2O) at room temperature. Peptic fragments were loaded onto a reverse phase trap column at 600 μL/min with buffer A and desalted for 1 min (600 μL buffer A). The desalted fragments were separated by a C18 column with a linear gradient of 8% to 35% buffer B (95% acetonitrile, 5% H₂O, 0.0025% TFA) at 100 L/min over 20 min and analyzed by mass spectrometry. Mass spectrometric analyses were carried out using an LTQ™ Orbitrap Fusion Lumos mass spectrometer (Thermo Fisher Scientific) with the capillary temperature at 275° C., resolution 150,000, and mass range (m/z) 300-1,800. BioPharma Finder 3.0 (Thermo Fisher Scientific) was used for the peptide identification of non-deuterated samples prior to the HDX experiments. HDExaminer version 2.5 (Sierra Analytics, Modesto, CA) was used to extract centroid values from the MS raw data files for the HDX experiments.

Incubation of hK2 antibodies, hu11B6, KL2B494, KL2B467, KL2B30, KL2B413 and KL2B53 with soluble hK2 protein resulted in different patterns of hydrogen exchange and overall protection. The protected segments were mapped onto the sequence of hK2 antigen to visualize the binding epitopes. KL2B494, KL2B467 and KL2B30 bound to common sequences of (i) residues 174-178 (SEQ ID NO: 111, KVTEF) (e.g., KL2B494, KL2B467 and KL2B30 bound at least three of the residues of SEQ ID NO: 111, namely, the KVT residues at 174-176) and (ii) residues 230-234 (SEQ ID NO: 112, HYRKW) (e.g., KL2B494, KL2B467 and KL2B30 bound at least three of the residues of SEQ ID NO: 112, namely, the HYR residues at 230-232). KL2B413 also bound all residues of SEQ ID NO: 111 and the KW residues of SEQ ID NO: 112. An embodiment of the present invention provides an isolated protein comprising an antigen binding domain that binds hK2, wherein said antigen binding domain binds to hK2 within epitopes having sequences of SEQ ID NO: 111 and SEQ ID NO: 112; for example, said antigen binding domain binds to all residues, or at least four residues, or at least three residues of SEQ ID NO: 111 and binds to all residues, or at least four residues, or at least three residues of SEQ ID NO: 112.

KL2B53 showed a different pattern of protection and bound to a sequence consisting of residues 27-32 (Seq ID NO: 113, SHGWAH), 60-75 (SEQ ID NO: 114, RHNLFEPEDTGQRVP) and 138-147 (SEQ ID NO: 115, GWGSIEPEE).

According to an embodiment, an isolated anti-hK2/anti-CD3 protein (e.g., hu11B6, KL2B494, KL2B467, KL2B30, KL2B413, or KL2B53) comprises an hk2-specific antigen binding domain that specifically binds to a discontinuous epitope (i.e., epitopes whose residues are distantly placed in the sequence) of hK2 comprising one or more amino acid sequences selected from the group consisting of SEQ ID NO: 111, 112, 113, 114, and 115.

The paratope of anti-hK2 antibodies hu11B6, KL2B494, KL2B467, KL2B413 and anti-hK2/CD3 bispecific antibodies KLCB113 and KLCB80 were identified based on significant differences in deuterium uptake from the HDExaminer residue plots. KL2BB494 comprises three paratope regions two of which are located in the KL2B494 heavy chain variable domain (GFTFSH (SEQ ID NO: 455) and TAVYYCAKPHIVMVTAL (SEQ ID NO: 456)) and a single paratope region located within the light chain variable domain (YDDSDRPSGIPER (SEQ ID NO: 457)). KL2B467 comprises three paratope regions, two of which are located in the KL2B467 heavy chain variable domain (FTFSY (SEQ ID NO: 458) and GSYWAFDY (SEQ ID NO: 459)) and a single paratope region within the light chain variable domain (DNSD (SEQ ID NO: 460)). Hu11B6 comprises a single epitope region located in the heavy chain (GNSITSDYA (SEQ ID NO: 461)). KL2B413 comprises two paratope regions located in the heavy chain variable domain (GFTF (SEQ ID NO: 462) and ARDQNYDIL (SEQ ID NO: 463)). KL2B30 of bispecific KLCB80 comprise a paratope region locate in the heavy chain (comprising amino acid residues TIF and VTPNF (SEQ ID NO: 464)) and a paratope region located in the light chain (YAASTLQSG (SEQ ID NO: 465)). KL2B53 of bispecific KLCB113 comprise a single paratope region locate in the heavy chain (comprising amino acid residues ESGWSHY (SEQ ID NO: 466)).

### Example 25—MMAF Conjugation to KL2B30 Fab

Immunoconjugates of the present invention were made by conjugating the KL2B30 Fab (identified as KL2B997) to MMAF. Conjugation was performed via random conjugation and site-specific conjugation; such methods are described, for example, in WO2020/229974. As described herein, KL2B997 (the Fab of KL2B30) comprises a HCDR1, a HCDR2, a HCDR3, a LCDR1, a LCDR2 and a LCDR3 of SEQ ID NOs: 170, 171, 172, 173, 174 and 175, respectively; and KL2B997 comprises a VH of SEQ ID NO: 162 and a VL of SEQ ID NO: 163.

In FIG. 1, the term “DAR” is “drug-to-antibody ratio”, which refers to the number of drug molecules, i.e., MMAF, attached to the antibody moiety, i.e., KL2B997. The number of drug molecules bound per antibody moiety or the degree of labeling is a parameter commonly used in the art and is designated “DAR” for “drug-antibody ratio.”

To evaluate target cell binding of the KL2B30 Fab with different conjugation variants, VCaP cells were used as hK2-positive target cells and DU145 cells were used as hK2-negative cells. KL2B30 parental antibody (comprising a heavy chain and light chain of SEQ ID NO: 210 and SEQ ID NO: 221, respectively) and hu11B6 (an anti-hK2 antibody, also referred to herein as h11B6, comprising a heavy chain and light chain of SEQ ID NO: 203 and SEQ ID NO: 215, respectively) were included as positive controls. The h11B6 antibody is described in U.S. Pat. No. 10,100,125, which is incorporated by reference herein. An anti-Human F(ab′)2 fragment specific secondary detection antibody was used for detecting Fab-based binding. Test antibodies were incubated with target cells at 4° C. for 60 minutes and detected by secondary staining. Cell binding was quantified by flow cytometry based on secondary antibody binding signal and gated from on live cells. Results indicated positive and dose-dependent cell binding using all three tested methods, which included un-conjugated KL2B997, random conjugation of KL2B997 and site-specific conjugation of KL2B997 (depicted in FIG. 1 as KL2B997*, KL2B997 NHS and KL2B997 SORT). KL2B30 Fab was shown to bind specifically to hK2-positive VCaP cells and not to hK2-negative DU145 cells. As shown in FIG. 1, results showed the KL2B30 Fab binds to hK2-expressing VCaP cells and can internalize and trigger internalization-based cell killing when conjugated with MMAF.

The immunoconjugates described above were made via random conjugation and site-specific conjugation. For site-specific conjugation, 2.1 mgs of h11B6 at 2 mg/mL in 1× dPBS (Thermo Fisher 14190144) was deglycosylated with 5 ul of Rapid PNGaseF (NEB #P0711S) overnight at 37° C. Bacterial transglutaminase (bTG; Activa TI from Ajinomoto) was added to 30% w/v along with a 1000× molar excess of the amino-PEG4-(PEG3-azide)2 branched substrate (CP-2051 Conju-Probe LLC) and incubated overnight at room temperature. The azide-modified mAb was purified on an AKTA Avant instrument equipped with a 1 ml Mabselect column (GE 11003493) and exchanged into 1× dPBS with 10 mL Zeba desalting columns (Thermo Fisher). DBCO-PEG4-vc-PAB-MMAF (Levena Biopharma) was added in 10× molar excess and reaction was monitored by LC-MS until reaching drug:antibody ratio of 4. The final h11B6-vcMMAF ADC was purified on a Zeba desalting column followed by concentration and diafiltration with an Amicon concentrator.

For random conjugation, KL2B30 was first reacted with to NHS-PEG4-azide (Thermo Fisher Cat #26130). To 1.5 mgs of KL2B30 at 1 mg/ml in 1× dPBS was added 30 ul of 1M pH 9 sodium bicarbonate (BDH #144-55-8) and immediately followed by the addition of a 7× molar ratio of NHS-PEG4-azide. Reaction was monitored by mass spectrometry until the degree of labeling reached 2-2.5 and then quenched by addition of Tris pH 8.5 (Teknova T1085) to a final concentration of 100 mM. Following removal of unreacted NHS-PEG4-azide, a 10× molar excess of DBCO-PEG4-vc-PAB-MMAF was added and incubated for 1 hour at 37° C. Final ADC (DAR=2.7) was purified by 10 ml Zeba desalt column followed by concentration and diafiltration with an Amicon concentrator.

The KL2B997 site-specific ADC (antibody-drug conjugate) was prepared by conjugation via the sortase tag. 1 mg of KL2B997 at 1 mg/ml was incubated with S. pyogenes sortase A enzyme (2 uM) [reference to Chen et al. PNAS 2011:11399], and an excess of Gly3-vcMMAF (Levena Biopharma) in 50 mM Tris pH 7.5, 150 mM NaCl, 10 mM CaCl2) buffer. Reaction was incubated at for 1 hour at 37° C. The conjugate as purified on a 1 mL HisTrap column (Cytiva 17524701) on an AKTA Avant instrument. The ADC was further purified over a 24 ml Superdex 75 10/300 (Cytiva 29148721) in 1×dPBS to remove any aggregate or residual substrates.

KL2B997 randomly conjugated ADC was prepared by addition of NHS-PEG4-azide followed by reaction with DBCO-vcMMAF as described above.

### Example 26—TOPA-KL2B1251 Fab and NODA-GA-KL2B1251 Fab

Methods of conjugating a KL2B30 Fab (identified as KL2B1251) to TOPA and NODA-GA are provided below.

### Preparation of TOPA-KL2B1251.

Random TOPA modification of Fab: KL2B1251 Fab was diluted to 1 mg/ml in sterile 1×dPBS. Adjusted the pH of the mAb to 9.0 with sodium bicarbonate buffer pH 9 (VWR 144-55-8). Then, 8× molar excess of TOPA (TOPA-phenyl-NCS (intermediate); 50 mM stock dissolved in DMSO) was added, and the pool was incubated at room temperature without shaking for approximately 1 hour. The addition of TOPA was monitored by intact mass ESI-TOF LC-MS on an Agilent G224 instrument until the CAR value was between 1.5-2.0. Pool was quenched by addition of 1M Tris pH 8.5 (Teknova T1085) to a final concentration of 100 mM. Excess free chelator was removed by successive rounds of desalting and eluting 15 mls of conjugate pool at a time over a 55 ml HiPrep 26/10 desalting column (17508701—Cytiva). Equilibration and elution all performed in 1×dPBS. Sample was then concentrated to 2 mls. To confirm no excess chelator was present, 3× rounds of sample dilution to 15 mls followed by concentration to 1 ml using a 50,000 MWCO Amicon concentrator device was performed. The final conjugate was confirmed to be monomeric by analytical size exclusion chromatography on a Tosoh TSKgel G3000SWxl 7.8 mm×30 cm, 5 u column; column temperature: room temperature; the column was eluted 0.2M sodium phosphate pH 6.8; flow rate: 0.8 mL/min; 18 min run; injection volume: 18 ul.

### Preparation of NODA-GA-KL2B1251.

Random NODA modification of Fab: KL2B1251 Fab was diluted to 1 mg/ml in sterile 1×dPBS. Rotated entire pool with Chelex resin (BioRad 1432832) for 30 mins at RT, then filtered out the resin prior to the next steps with a sterile filter. Directly before conjugation, added chelex treated 1M sodium bicarbonate pH 9.0 (VWR 1M solution pH 9.0 Cat #144-55-8). Added 30×NODA-GA-NHS ester (2,2′-(7-(1-carboxy-4-((2,5-dioxopyrrolidin-1-yl)oxy)-4-oxobutyl)-1,4,7-triazonane-1,4-diyl)diacetic acid; Chematech Cat #C098 in 50 mM DMSO). Incubated at room temperature without shaking for ˜1 hour. The addition of NODA was monitored by intact mass ESI-TOF LC-MS on an Agilent G224 instrument until the CAR value was between 1.5-2.0. Pool was quenched by addition of 1M Tris pH 8.5 (Teknova T1085) to a final concentration of 100 mM. To keep the prep as metal free as possible, purification was performed only via 30,000 MWCO concentrator devices. The pool was split over six 30,000 MWCO Amicon concentrators—each holding ˜6 mls of volume and topped off with chelex PBS. Concentrators were spun for 8 minutes at 3800× g, leaving ˜1 ml conjugate in each. Each concentrator was topped off with chelex PBS. Process was repeated 5 additional times. The final conjugate was confirmed to be monomeric by analytical size exclusion chromatography on a Tosoh TSKgel G3000SWxl 7.8 mm×30 cm, 5 u column; column temperature: room temperature; the column was eluted 0.2M sodium phosphate pH 6.8; flow rate: 0.8 mL/min; 18 min run; injection volume: 18 ul.

Using methods described in Example 25, to evaluate target cell binding of the KL2B1251 Fab with different conjugation variants, VCaP cells were used as hK2-positive target cells and DU145 cells were used as hK2-negative cells. As shown in FIG. 3, the KL2B1251 Fab and KL2B1251-NODA-GA were shown to bind specifically to hK2-positive VCaP cells (FIG. 3A) and not to hK2-negative DU145 cells (FIG. 3B).

### Example 27—Pharmacokinetic (PK) Study of KL2B1251-NOTA and KL2B1251-TOPA

The objective of this study was to elucidate the pharmacokinetic properties of the NODA-GA-KL2B1251 Fab as described herein (referred to in this example as “KL2B1251-NOTA”) and the TOPA-KL2B1251 Fab as described herein (referred to in this example as “KL2B1251-TOPA”) following a single IV in male cynomolgus monkeys. The first dosing occurred on Day 1. KL2B1251-NOTA was administered to 4 male cynomolgus monkeys by intravenous (IV) administration as a slow bolus injection over approximately one minute, at a target dose of 1 mg/kg. KL2B1251-TOPA was also administered to 4 male cynomolgus monkeys by intravenous (IV) administration as a slow bolus injection over approximately one minute, at a target dose of 1 mg/kg. The individual body weights and doses administered are provided in FIG. 5.

Individual and mean (SD) serum PK estimates of KL2B1251-NOTA and KL2B1251-TOPA following the single IV bolus dose are summarized in FIG. 6. Mean (SD) serum concentrations of KL2B1251-NOTA and KL2B1251-TOPA versus time for male monkeys is illustrated in FIG. 7. Serum KL2B1251-NOTA and KL2B1251-TOPA concentrations were below the lowest quantifiable concentration in all samples collected prior to the dose administration in KL2B1251-NOTA and KL2B1251-TOPA dose groups. KL2B1251-NOTA exposure (in terms of AUCinf) was 1.72-fold higher than KL2B1251-TOPA. KL2B1251-NOTA exhibited faster serum clearance and lower volume of distribution as compared to KL2B1251-TOPA. Terminal T_{1/2} for KL2B1251-NOTA was slightly shorter than KL2B1251-TOPA. Following a single IV dose of KL2B1251-NOTA and KL2B1251-TOPA, mean values were 21555.77 and 16933.83 ng/mL for Cₘₐₓ, 1440.71 and 837.55 ng day/mL for AUC_{inf}, 711.23 and 1271.46 mL/day/kg for CL, and 0.60 (14.4 hours) and 0.85 (20.4 hours) days for T_{1/2}, respectively.

It is understood that the examples and embodiments described herein are for illustrative purposes only, and that changes could be made to the embodiments described above without departing from the broad inventive concept thereof. It is understood, therefore, that this invention is not limited to the particular embodiments disclosed, but it is intended to cover modifications within the spirit and scope of the invention as defined by the appended claims.

1. An immunoconjugate comprising: a radiometal complex conjugated to an antigen binding domain with binding specificity for kallikrein related peptidase 2 (hK2), wherein the radiometal complex comprises ⁶⁴Cu bound to a chelator, and wherein the chelator is conjugated to the antigen binding domain with binding specificity for kallikrein related peptidase 2 (hK2).

2. The immunoconjugate of claim 1, wherein the chelator is 1,4,7,10-tetraazacyclododecane-1,4,7,10,tetraacetic acid (DOTA), S-2-(4-isothiocyanatobenzyl)-1,4,7-triazacyclononane-1,4,7-triacetic acid (NOTA), 1,4,8,11-tetraazacyclodocedan-1,4,8,11-tetraacetic acid (TETA), 3,6,9,15-tetraazabicyclo[9.3.1]-pentadeca-1(15),11,13-triene-4-(S)-(4-isothiocyanatobenzyl)-3,6,9-triacetic acid (PCTA), 5-S-(4-aminobenzyl)-1-oxa-4,7,10-triazacyclododecane-4,7,10-tris(acetic acid) (DO3A), or a derivative thereof.

3. The immunoconjugate of claim 1, wherein the chelator is NOTA or a derivative thereof, such as NODA-GA, NODA-GA(tButyl)₃, Di-t-butyl-NOTA, NOTA-thiosemicarbazide, NODA-MPAA, or NODA-MPAEM.

4. The immunoconjugate according to claim 1, wherein the antigen binding domain with binding specificity for hK2 is a Fab.

5. The immunoconjugate according to claim 1, wherein the antigen binding domain comprises the HCDR1, the HCDR2, the HCDR3, the LCDR1, the LCDR2 and the LCDR3 of SEQ ID NO: 170 (SYYWS), SEQ ID NO: 171 (YIYYSGSTNYNPSLKS), SEQ ID NO: 172 (TTIFGVVTPNFYYGMDV), SEQ ID NO: 173 (RASQGISSYLA), SEQ ID NO: 174 (AASTLQS) and SEQ ID NO: 175 (QQLNSYPLT), respectively.

6. The immunoconjugate according to claim 1, wherein the antigen binding domain that binds hK2 comprises a VH which is at least 80% (e.g. at least 85%, at least 90%, at least 95%, at least 99% or 100%) identical to the VH of SEQ ID NO: 162 (QVQLQESGPGLVKPSETLSLTCTVSGGSISSYYWSWIRQPPGKGLEWIGYIYYSGSTN YNPSLKSRVTISVDTSKNQFSLKLSSVTAADTAVYYCAGTTIFGVVTPNFYYGMDVW GQGTTVTVSS), and a VL which is at least 80% (e.g. at least 85%, at least 90%, at least 95%, at least 99% or 100%) identical to the VL of SEQ ID NO: 163 (DIQMTQSPSFLSASVGDRVTITCRASQGISSYLAWYQQKPGKAPKFLIYAASTLQSG VPSRFSGSGSGTEFTLTISSLQPEDFATYYCQQLNSYPLTFGGGTKVEIK).

7. The immunoconjugate according to claim 1, wherein the antigen binding domain that binds hK2 comprises the VH of SEQ ID NO: 162 and the VL of SEQ ID NO: 163.

8. The immunoconjugate according to claim 1, wherein the antigen binding domain is a Fab that comprises:
a. a HCDR1, a HCDR2, a HCDR3, a LCDR1, a LCDR2 and a LCDR3 of SEQ ID NOs: 170, 171, 172, 173, 174 and 175, respectively; and/or
b. a VH of SEQ ID NO: 162 and a VL of SEQ ID NO: 163.

9. The immunoconjugate according to claim 1, wherein the immunoconjugate is a short half-life immunoconjugate.

10. An immunoconjugate comprising: a radiometal complex conjugated to an antigen binding domain with binding specificity for kallikrein related peptidase 2 (hK2), wherein:
the radiometal complex comprises ⁶⁴Cu bound to a chelator that is NOTA or a derivative thereof, such as NODA-GA, NODA-GA(tButyl)₃, Di-t-butyl-NOTA, NOTA-thiosemicarbazide, NODA-MPAA, or NODA-MPAEM,
wherein the chelator is conjugated to the antigen binding domain with binding specificity for kallikrein related peptidase 2 (hK2), and
wherein the antigen binding domain comprises:
(a) the HCDR1, the HCDR2, the HCDR3, the LCDR1, the LCDR2 and the LCDR3 of SEQ ID NO: 170 (SYYWS), SEQ ID NO: 171 (YIYYSGSTNYNPSLKS), SEQ ID NO: 172 (TTIFGVVTPNFYYGMDV), SEQ ID NO: 173 (RASQGISSYLA), SEQ ID NO: 174 (AASTLQS) and SEQ ID NO: 175 (QQLNSYPLT), respectively; and/or
(b) a VH which is at least 95%, at least 99% or 100% identical to the VH of SEQ ID NO: 162, and a VL which is at least 95%, at least 99% or 100% identical to the VL of SEQ ID NO: 163.

11. A method of detecting the presence of prostate cancer in a subject, comprising administering an immunoconjugate according to claim 1 to a subject suspected to have prostate cancer and visualizing biological structures to which the immunoconjugate is bound (e.g., by computerized tomography or positron emission tomography), thereby detecting the presence of prostate cancer.

12. A method of detecting the progress of cancer treatment in a subject, comprising administering an immunoconjugate according to claim 1 to the subject and visualizing biological structures to which the conjugate is bound (e.g., by computerized tomography or positron emission tomography), thereby detecting the progress of prostate cancer treatment in the subject.

13. The method of claim 11 comprising using positron emission tomography (PET) imaging to visualize the biological structures to which the immunoconjugate is bound.

14. The immunoconjugate of claim 3, wherein the chelator is NODA-GA.

15. The immunoconjugate of claim 10, wherein the chelator is NODA-GA.

16. The method of claim 12 comprising using positron emission tomography (PET) imaging to visualize the biological structures to which the immunoconjugate is bound.


SEQUENCE LISTING (475 sequences)

SEQ ID NO: 1 - protein, 111 aa, synthetic construct
  REGION 1..111: note = Description of Artificial Sequence: Synthetic polypeptide
DIVLTQSPDSLAVSLGERATINCKASESVEYFGTSLMHWYQQKPGQPPKLLIYAASNRESGVPDRFSGSGSGTDFTLTIQSVQAEDVSVYFCQQTRKVPYTFGQGTKLEIK

SEQ ID NO: 2 - protein, 111 aa, synthetic construct
  REGION 1..111: note = Description of Artificial Sequence: Synthetic polypeptide
DIVLTQSPDSLAVSLGERATINCKASESVEYFGTSLMHWYQQKPGQPPKLLIYAASNRESGVPDRFSGSGSGTDFTLTISSLQAEDVAVYYCQQTRKVPYTFGQGTKLEIK

SEQ ID NO: 3 - protein, 111 aa, synthetic construct
  REGION 1..111: note = Description of Artificial Sequence: Synthetic polypeptide
DIVLTQSPDSLAVSLGERATINCKASESVEYFGTSLMHWYQQKPGQPPKLLIYAASNRESGVPDRFSGSGSGTDFTLTISSVQAEDVAVYYCQQTRKVPYTFGQGTKLEIK

SEQ ID NO: 4 - protein, 117 aa, synthetic construct
  REGION 1..117: note = Description of Artificial Sequence: Synthetic polypeptide
QVQLQESGPGLVKPSDTLSLTCAVSGNSITSDYAWNWIRQFPGKGLEWMGYISYSGSTTYNPSLKSRVTISRDTSKNQFSLKLSSVTPVDTAVYYCATGYYYGSGFWGQGTLVTVSS

SEQ ID NO: 5 - protein, 117 aa, synthetic construct
  REGION 1..117: note = Description of Artificial Sequence: Synthetic polypeptide
QVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSS

SEQ ID NO: 6 - protein, 117 aa, synthetic construct
  REGION 1..117: note = Description of Artificial Sequence: Synthetic polypeptide
QVQLQESGPGLVKPSDTLSLTCAVSGNSITSDYAWNWIRQFPGKGLEWIGYISYSGSTTYNPSLKSRVTISRDTSKNQFSLKLSSVTPVDTAVYYCATGYYYGSGFWGQGTLVTVSS

SEQ ID NO: 7 - protein, 20 aa, synthetic construct
  REGION 1..20: note = Description of Artificial Sequence: Synthetic peptide
GGSEGKSSGSGSESKSTGGS

SEQ ID NO: 8 - protein, 248 aa, synthetic construct
  REGION 1..248: note = Description of Artificial Sequence: Synthetic polypeptide
QVQLQESGPGLVKPSDTLSLTCAVSGNSITSDYAWNWIRQFPGKGLEWMGYISYSGSTTYNPSLKSRVTISRDTSKNQFSLKLSSVTPVDTAVYYCATGYYYGSGFWGQGTLVTVSSGGSEGKSSGSGSESKSTGGSDIVLTQSPDSLAVSLGERATINCKASESVEYFGTSLMHWYQQKPGQPPKLLIYAASNRESGVPDRFSGSGSGTDFTLTIQSVQAEDVSVYFCQQTRKVPYTFGQGTKLEIK

SEQ ID NO: 9 - protein, 248 aa, synthetic construct
  REGION 1..248: note = Description of Artificial Sequence: Synthetic polypeptide
QVQLQESGPGLVKPSDTLSLTCAVSGNSITSDYAWNWIRQFPGKGLEWMGYISYSGSTTYNPSLKSRVTISRDTSKNQFSLKLSSVTPVDTAVYYCATGYYYGSGFWGQGTLVTVSSGGSEGKSSGSGSESKSTGGSDIVLTQSPDSLAVSLGERATINCKASESVEYFGTSLMHWYQQKPGQPPKLLIYAASNRESGVPDRFSGSGSGTDFTLTISSLQAEDVAVYYCQQTRKVPYTFGQGTKLEIK

SEQ ID NO: 10 - protein, 248 aa, synthetic construct
  REGION 1..248: note = Description of Artificial Sequence: Synthetic polypeptide
QVQLQESGPGLVKPSDTLSLTCAVSGNSITSDYAWNWIRQFPGKGLEWIGYISYSGSTTYNPSLKSRVTISRDTSKNQFSLKLSSVTPVDTAVYYCATGYYYGSGFWGQGTLVTVSSGGSEGKSSGSGSESKSTGGSDIVLTQSPDSLAVSLGERATINCKASESVEYFGTSLMHWYQQKPGQPPKLLIYAASNRESGVPDRFSGSGSGTDFTLTISSVQAEDVAVYYCQQTRKVPYTFGQGTKLEIK

SEQ ID NO: 11 - protein, 248 aa, synthetic construct
  REGION 1..248: note = Description of Artificial Sequence: Synthetic polypeptide
QVQLQESGPGLVKPSDTLSLTCAVSGNSITSDYAWNWIRQFPGKGLEWMGYISYSGSTTYNPSLKSRVTISRDTSKNQFSLKLSSVTPVDTAVYYCATGYYYGSGFWGQGTLVTVSSGGSEGKSSGSGSESKSTGGSDIVLTQSPDSLAVSLGERATINCKASESVEYFGTSLMHWYQQKPGQPPKLLIYAASNRESGVPDRFSGSGSGTDFTLTISSVQAEDVAVYYCQQTRKVPYTFGQGTKLEIK

SEQ ID NO: 12 - protein, 248 aa, synthetic construct
  REGION 1..248: note = Description of Artificial Sequence: Synthetic polypeptide
DIVLTQSPDSLAVSLGERATINCKASESVEYFGTSLMHWYQQKPGQPPKLLIYAASNRESGVPDRFSGSGSGTDFTLTIQSVQAEDVSVYFCQQTRKVPYTFGQGTKLEIKGGSEGKSSGSGSESKSTGGSQVQLQESGPGLVKPSDTLSLTCAVSGNSITSDYAWNWIRQFPGKGLEWMGYISYSGSTTYNPSLKSRVTISRDTSKNQFSLKLSSVTPVDTAVYYCATGYYYGSGFWGQGTLVTVSS

SEQ ID NO: 13 - protein, 248 aa, synthetic construct
  REGION 1..248: note = Description of Artificial Sequence: Synthetic polypeptide
DIVLTQSPDSLAVSLGERATINCKASESVEYFGTSLMHWYQQKPGQPPKLLIYAASNRESGVPDRFSGSGSGTDFTLTISSLQAEDVAVYYCQQTRKVPYTFGQGTKLEIKGGSEGKSSGSGSESKSTGGSQVQLQESGPGLVKPSDTLSLTCAVSGNSITSDYAWNWIRQFPGKGLEWIGYISYSGSTTYNPSLKSRVTISRDTSKNQFSLKLSSVTPVDTAVYYCATGYYYGSGFWGQGTLVTVSS

SEQ ID NO: 14 - protein, 248 aa, synthetic construct
  REGION 1..248: note = Description of Artificial Sequence: Synthetic polypeptide
DIVLTQSPDSLAVSLGERATINCKASESVEYFGTSLMHWYQQKPGQPPKLLIYAASNRESGVPDRFSGSGSGTDFTLTISSLQAEDVAVYYCQQTRKVPYTFGQGTKLEIKGGSEGKSSGSGSESKSTGGSQVQLQESGPGLVKPSDTLSLTCAVSGNSITSDYAWNWIRQFPGKGLEWMGYISYSGSTTYNPSLKSRVTISRDTSKNQFSLKLSSVTPVDTAVYYCATGYYYGSGFWGQGTLVTVSS

SEQ ID NO: 15 - protein, 248 aa, synthetic construct
  REGION 1..248: note = Description of Artificial Sequence: Synthetic polypeptide
DIVLTQSPDSLAVSLGERATINCKASESVEYFGTSLMHWYQQKPGQPPKLLIYAASNRESGVPDRFSGSGSGTDFTLTISSVQAEDVAVYYCQQTRKVPYTFGQGTKLEIKGGSEGKSSGSGSESKSTGGSQVQLQESGPGLVKPSDTLSLTCAVSGNSITSDYAWNWIRQFPGKGLEWIGYISYSGSTTYNPSLKSRVTISRDTSKNQFSLKLSSVTPVDTAVYYCATGYYYGSGFWGQGTLVTVSS

SEQ ID NO: 16 - protein, 248 aa, synthetic construct
  REGION 1..248: note = Description of Artificial Sequence: Synthetic polypeptide
DIVLTQSPDSLAVSLGERATINCKASESVEYFGTSLMHWYQQKPGQPPKLLIYAASNRESGVPDRFSGSGSGTDFTLTISSVQAEDVAVYYCQQTRKVPYTFGQGTKLEIKGGSEGKSSGSGSESKSTGGSQVQLQESGPGLVKPSDTLSLTCAVSGNSITSDYAWNWIRQFPGKGLEWMGYISYSGSTTYNPSLKSRVTISRDTSKNQFSLKLSSVTPVDTAVYYCATGYYYGSGFWGQGTLVTVSS

SEQ ID NO: 17 - protein, 248 aa, synthetic construct
  REGION 1..248: note = Description of Artificial Sequence: Synthetic polypeptide
DIVLTQSPDSLAVSLGERATINCKASESVEYFGTSLMHWYQQKPGQPPKLLIYAASNRESGVPDRFSGSGSGTDFTLTIQSVQAEDVSVYFCQQTRKVPYTFGQGTKLEIKGGSEGKSSGSGSESKSTGGSQVQLQESGPGLVKPSDTLSLTCAVSGNSITSDYAWNWIRQFPGKGLEWIGYISYSGSTTYNPSLKSRVTISRDTSKNQFSLKLSSVTPVDTAVYYCATGYYYGSGFWGQGTLVTVSS

SEQ ID NO: 18 - protein, 248 aa, synthetic construct
  REGION 1..248: note = Description of Artificial Sequence: Synthetic polypeptide
QVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSSGGSEGKSSGSGSESKSTGGSDIVLTQSPDSLAVSLGERATINCKASESVEYFGTSLMHWYQQKPGQPPKLLIYAASNRESGVPDRFSGSGSGTDFTLTISSVQAEDVAVYYCQQTRKVPYTFGQGTKLEIK

SEQ ID NO: 19 - protein, 248 aa, synthetic construct
  REGION 1..248: note = Description of Artificial Sequence: Synthetic polypeptide
QVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSSGGSEGKSSGSGSESKSTGGSDIVLTQSPDSLAVSLGERATINCKASESVEYFGTSLMHWYQQKPGQPPKLLIYAASNRESGVPDRFSGSGSGTDFTLTIQSVQAEDVSVYFCQQTRKVPYTFGQGTKLEIK

SEQ ID NO: 20 - protein, 248 aa, synthetic construct
  REGION 1..248: note = Description of Artificial Sequence: Synthetic polypeptide
QVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSSGGSEGKSSGSGSESKSTGGSDIVLTQSPDSLAVSLGERATINCKASESVEYFGTSLMHWYQQKPGQPPKLLIYAASNRESGVPDRFSGSGSGTDFTLTISSLQAEDVAVYYCQQTRKVPYTFGQGTKLEIK

SEQ ID NO: 21 - protein, 248 aa, synthetic construct
  REGION 1..248: note = Description of Artificial Sequence: Synthetic polypeptide
DIVLTQSPDSLAVSLGERATINCKASESVEYFGTSLMHWYQQKPGQPPKLLIYAASNRESGVPDRFSGSGSGTDFTLTIQSVQAEDVSVYFCQQTRKVPYTFGQGTKLEIKGGSEGKSSGSGSESKSTGGSQVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSS

SEQ ID NO: 22 - protein, 248 aa, synthetic construct
  REGION 1..248: note = Description of Artificial Sequence: Synthetic polypeptide
DIVLTQSPDSLAVSLGERATINCKASESVEYFGTSLMHWYQQKPGQPPKLLIYAASNRESGVPDRFSGSGSGTDFTLTISSLQAEDVAVYYCQQTRKVPYTFGQGTKLEIKGGSEGKSSGSGSESKSTGGSQVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSS

SEQ ID NO: 23 - protein, 248 aa, synthetic construct
  REGION 1..248: note = Description of Artificial Sequence: Synthetic polypeptide
DIVLTQSPDSLAVSLGERATINCKASESVEYFGTSLMHWYQQKPGQPPKLLIYAASNRESGVPDRFSGSGSGTDFTLTISSVQAEDVAVYYCQQTRKVPYTFGQGTKLEIKGGSEGKSSGSGSESKSTGGSQVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSS

SEQ ID NO: 24 - protein, 19 aa, synthetic construct
  REGION 1..19: note = Description of Artificial Sequence: Synthetic peptide
MAWVWTLLFLMAAAQSIQA

SEQ ID NO: 25 - protein, 45 aa, synthetic construct
  REGION 1..45: note = Description of Artificial Sequence: Synthetic polypeptide
TSTPAPRPPTPAPTIASQPLSLRPEACRPAAGGAVHTRGLDFACD

SEQ ID NO: 26 - protein, 24 aa, synthetic construct
  REGION 1..24: note = Description of Artificial Sequence: Synthetic peptide
IYIWAPLAGTCGVLLLSLVITLYC

SEQ ID NO: 27 - protein, 42 aa, Homo sapiens
KRGRKKLLYIFKQPFMRPVQTTQEEDGCSCRFPEEEEGGCEL

SEQ ID NO: 28 - protein, 112 aa, Homo sapiens
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 267 aa, synthetic construct
  REGION 1..267: note = Description of Artificial Sequence: Synthetic polypeptide
MAWVWTLLFLMAAAQSIQAQVQLQESGPGLVKPSDTLSLTCAVSGNSITSDYAWNWIRQFPGKGLEWMGYISYSGSTTYNPSLKSRVTISRDTSKNQFSLKLSSVTPVDTAVYYCATGYYYGSGFWGQGTLVTVSSGGSEGKSSGSGSESKSTGGSDIVLTQSPDSLAVSLGERATINCKASESVEYFGTSLMHWYQQKPGQPPKLLIYAASNRESGVPDRFSGSGSGTDFTLTIQSVQAEDVSVYFCQQTRKVPYTFGQGTKLEIK

SEQ ID NO: 30 - protein, 267 aa, synthetic construct
  REGION 1..267: note = Description of Artificial Sequence: Synthetic polypeptide
MAWVWTLLFLMAAAQSIQAQVQLQESGPGLVKPSDTLSLTCAVSGNSITSDYAWNWIRQFPGKGLEWMGYISYSGSTTYNPSLKSRVTISRDTSKNQFSLKLSSVTPVDTAVYYCATGYYYGSGFWGQGTLVTVSSGGSEGKSSGSGSESKSTGGSDIVLTQSPDSLAVSLGERATINCKASESVEYFGTSLMHWYQQKPGQPPKLLIYAASNRESGVPDRFSGSGSGTDFTLTISSLQAEDVAVYYCQQTRKVPYTFGQGTKLEIK

SEQ ID NO: 31 - protein, 267 aa, synthetic construct
  REGION 1..267: note = Description of Artificial Sequence: Synthetic polypeptide
MAWVWTLLFLMAAAQSIQAQVQLQESGPGLVKPSDTLSLTCAVSGNSITSDYAWNWIRQFPGKGLEWIGYISYSGSTTYNPSLKSRVTISRDTSKNQFSLKLSSVTPVDTAVYYCATGYYYGSGFWGQGTLVTVSSGGSEGKSSGSGSESKSTGGSDIVLTQSPDSLAVSLGERATINCKASESVEYFGTSLMHWYQQKPGQPPKLLIYAASNRESGVPDRFSGSGSGTDFTLTISSVQAEDVAVYYCQQTRKVPYTFGQGTKLEIK

SEQ ID NO: 32 - protein, 267 aa, synthetic construct
  REGION 1..267: note = Description of Artificial Sequence: Synthetic polypeptide
MAWVWTLLFLMAAAQSIQAQVQLQESGPGLVKPSDTLSLTCAVSGNSITSDYAWNWIRQFPGKGLEWMGYISYSGSTTYNPSLKSRVTISRDTSKNQFSLKLSSVTPVDTAVYYCATGYYYGSGFWGQGTLVTVSSGGSEGKSSGSGSESKSTGGSDIVLTQSPDSLAVSLGERATINCKASESVEYFGTSLMHWYQQKPGQPPKLLIYAASNRESGVPDRFSGSGSGTDFTLTISSVQAEDVAVYYCQQTRKVPYTFGQGTKLEIK

SEQ ID NO: 33 - protein, 267 aa, synthetic construct
  REGION 1..267: note = Description of Artificial Sequence: Synthetic polypeptide
MAWVWTLLFLMAAAQSIQADIVLTQSPDSLAVSLGERATINCKASESVEYFGTSLMHWYQQKPGQPPKLLIYAASNRESGVPDRFSGSGSGTDFTLTIQSVQAEDVSVYFCQQTRKVPYTFGQGTKLEIKGGSEGKSSGSGSESKSTGGSQVQLQESGPGLVKPSDTLSLTCAVSGNSITSDYAWNWIRQFPGKGLEWMGYISYSGSTTYNPSLKSRVTISRDTSKNQFSLKLSSVTPVDTAVYYCATGYYYGSGFWGQGTLVTVSS

SEQ ID NO: 34 - protein, 267 aa, synthetic construct
  REGION 1..267: note = Description of Artificial Sequence: Synthetic polypeptide
MAWVWTLLFLMAAAQSIQADIVLTQSPDSLAVSLGERATINCKASESVEYFGTSLMHWYQQKPGQPPKLLIYAASNRESGVPDRFSGSGSGTDFTLTISSLQAEDVAVYYCQQTRKVPYTFGQGTKLEIKGGSEGKSSGSGSESKSTGGSQVQLQESGPGLVKPSDTLSLTCAVSGNSITSDYAWNWIRQFPGKGLEWIGYISYSGSTTYNPSLKSRVTISRDTSKNQFSLKLSSVTPVDTAVYYCATGYYYGSGFWGQGTLVTVSS

SEQ ID NO: 35 - protein, 267 aa, synthetic construct
  REGION 1..267: note = Description of Artificial Sequence: Synthetic polypeptide
MAWVWTLLFLMAAAQSIQADIVLTQSPDSLAVSLGERATINCKASESVEYFGTSLMHWYQQKPGQPPKLLIYAASNRESGVPDRFSGSGSGTDFTLTISSLQAEDVAVYYCQQTRKVPYTFGQGTKLEIKGGSEGKSSGSGSESKSTGGSQVQLQESGPGLVKPSDTLSLTCAVSGNSITSDYAWNWIRQFPGKGLEWMGYISYSGSTTYNPSLKSRVTISRDTSKNQFSLKLSSVTPVDTAVYYCATGYYYGSGFWGQGTLVTVSS

SEQ ID NO: 36 - protein, 267 aa, synthetic construct
  REGION 1..267: note = Description of Artificial Sequence: Synthetic polypeptide
MAWVWTLLFLMAAAQSIQADIVLTQSPDSLAVSLGERATINCKASESVEYFGTSLMHWYQQKPGQPPKLLIYAASNRESGVPDRFSGSGSGTDFTLTISSVQAEDVAVYYCQQTRKVPYTFGQGTKLEIKGGSEGKSSGSGSESKSTGGSQVQLQESGPGLVKPSDTLSLTCAVSGNSITSDYAWNWIRQFPGKGLEWIGYISYSGSTTYNPSLKSRVTISRDTSKNQFSLKLSSVTPVDTAVYYCATGYYYGSGFWGQGTLVTVSS

SEQ ID NO: 37 - protein, 267 aa, synthetic construct
  REGION 1..267: note = Description of Artificial Sequence: Synthetic polypeptide
MAWVWTLLFLMAAAQSIQADIVLTQSPDSLAVSLGERATINCKASESVEYFGTSLMHWYQQKPGQPPKLLIYAASNRESGVPDRFSGSGSGTDFTLTISSVQAEDVAVYYCQQTRKVPYTFGQGTKLEIKGGSEGKSSGSGSESKSTGGSQVQLQESGPGLVKPSDTLSLTCAVSGNSITSDYAWNWIRQFPGKGLEWMGYISYSGSTTYNPSLKSRVTISRDTSKNQFSLKLSSVTPVDTAVYYCATGYYYGSGFWGQGTLVTVSS

SEQ ID NO: 38 - protein, 267 aa, synthetic construct
  REGION 1..267: note = Description of Artificial Sequence: Synthetic polypeptide
MAWVWTLLFLMAAAQSIQADIVLTQSPDSLAVSLGERATINCKASESVEYFGTSLMHWYQQKPGQPPKLLIYAASNRESGVPDRFSGSGSGTDFTLTIQSVQAEDVSVYFCQQTRKVPYTFGQGTKLEIKGGSEGKSSGSGSESKSTGGSQVQLQESGPGLVKPSDTLSLTCAVSGNSITSDYAWNWIRQFPGKGLEWIGYISYSGSTTYNPSLKSRVTISRDTSKNQFSLKLSSVTPVDTAVYYCATGYYYGSGFWGQGTLVTVSS

SEQ ID NO: 39 - protein, 267 aa, synthetic construct
  REGION 1..267: note = Description of Artificial Sequence: Synthetic polypeptide
MAWVWTLLFLMAAAQSIQAQVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSSGGSEGKSSGSGSESKSTGGSDIVLTQSPDSLAVSLGERATINCKASESVEYFGTSLMHWYQQKPGQPPKLLIYAASNRESGVPDRFSGSGSGTDFTLTISSVQAEDVAVYYCQQTRKVPYTFGQGTKLEIK

SEQ ID NO: 40 - protein, 267 aa, synthetic construct
  REGION 1..267: note = Description of Artificial Sequence: Synthetic polypeptide
MAWVWTLLFLMAAAQSIQAQVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSSGGSEGKSSGSGSESKSTGGSDIVLTQSPDSLAVSLGERATINCKASESVEYFGTSLMHWYQQKPGQPPKLLIYAASNRESGVPDRFSGSGSGTDFTLTIQSVQAEDVSVYFCQQTRKVPYTFGQGTKLEIK

SEQ ID NO: 41 - protein, 267 aa, synthetic construct
  REGION 1..267: note = Description of Artificial Sequence: Synthetic polypeptide
MAWVWTLLFLMAAAQSIQAQVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSSGGSEGKSSGSGSESKSTGGSDIVLTQSPDSLAVSLGERATINCKASESVEYFGTSLMHWYQQKPGQPPKLLIYAASNRESGVPDRFSGSGSGTDFTLTISSLQAEDVAVYYCQQTRKVPYTFGQGTKLEIK

SEQ ID NO: 42 - protein, 267 aa, synthetic construct
  REGION 1..267: note = Description of Artificial Sequence: Synthetic polypeptide
MAWVWTLLFLMAAAQSIQADIVLTQSPDSLAVSLGERATINCKASESVEYFGTSLMHWYQQKPGQPPKLLIYAASNRESGVPDRFSGSGSGTDFTLTIQSVQAEDVSVYFCQQTRKVPYTFGQGTKLEIKGGSEGKSSGSGSESKSTGGSQVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSS

SEQ ID NO: 43 - protein, 267 aa, synthetic construct
  REGION 1..267: note = Description of Artificial Sequence: Synthetic polypeptide
MAWVWTLLFLMAAAQSIQADIVLTQSPDSLAVSLGERATINCKASESVEYFGTSLMHWYQQKPGQPPKLLIYAASNRESGVPDRFSGSGSGTDFTLTISSLQAEDVAVYYCQQTRKVPYTFGQGTKLEIKGGSEGKSSGSGSESKSTGGSQVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSS

SEQ ID NO: 44 - protein, 267 aa, synthetic construct
  REGION 1..267: note = Description of Artificial Sequence: Synthetic polypeptide
MAWVWTLLFLMAAAQSIQADIVLTQSPDSLAVSLGERATINCKASESVEYFGTSLMHWYQQKPGQPPKLLIYAASNRESGVPDRFSGSGSGTDFTLTISSVQAEDVAVYYCQQTRKVPYTFGQGTKLEIKGGSEGKSSGSGSESKSTGGSQVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSS

SEQ ID NO: 45 - protein, 154 aa, synthetic construct
  REGION 1..154: note = Description of Artificial Sequence: Synthetic polypeptide
KRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 471 aa, synthetic construct
  REGION 1..471: note = Description of Artificial Sequence: Synthetic polypeptide
QVQLQESGPGLVKPSDTLSLTCAVSGNSITSDYAWNWIRQFPGKGLEWMGYISYSGSTTYNPSLKSRVTISRDTSKNQFSLKLSSVTPVDTAVYYCATGYYYGSGFWGQGTLVTVSSGGSEGKSSGSGSESKSTGGSDIVLTQSPDSLAVSLGERATINCKASESVEYFGTSLMHWYQQKPGQPPKLLIYAASNRESGVPDRFSGSGSGTDFTLTIQSVQAEDVSVYFCQQTRKVPYTFGQGTKLEIKTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 471 aa, synthetic construct
  REGION 1..471: note = Description of Artificial Sequence: Synthetic polypeptide
QVQLQESGPGLVKPSDTLSLTCAVSGNSITSDYAWNWIRQFPGKGLEWMGYISYSGSTTYNPSLKSRVTISRDTSKNQFSLKLSSVTPVDTAVYYCATGYYYGSGFWGQGTLVTVSSGGSEGKSSGSGSESKSTGGSDIVLTQSPDSLAVSLGERATINCKASESVEYFGTSLMHWYQQKPGQPPKLLIYAASNRESGVPDRFSGSGSGTDFTLTISSLQAEDVAVYYCQQTRKVPYTFGQGTKLEIKTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 471 aa, synthetic construct
  REGION 1..471: note = Description of Artificial Sequence: Synthetic polypeptide
QVQLQESGPGLVKPSDTLSLTCAVSGNSITSDYAWNWIRQFPGKGLEWIGYISYSGSTTYNPSLKSRVTISRDTSKNQFSLKLSSVTPVDTAVYYCATGYYYGSGFWGQGTLVTVSSGGSEGKSSGSGSESKSTGGSDIVLTQSPDSLAVSLGERATINCKASESVEYFGTSLMHWYQQKPGQPPKLLIYAASNRESGVPDRFSGSGSGTDFTLTISSVQAEDVAVYYCQQTRKVPYTFGQGTKLEIKTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 471 aa, synthetic construct
  REGION 1..471: note = Description of Artificial Sequence: Synthetic polypeptide
QVQLQESGPGLVKPSDTLSLTCAVSGNSITSDYAWNWIRQFPGKGLEWMGYISYSGSTTYNPSLKSRVTISRDTSKNQFSLKLSSVTPVDTAVYYCATGYYYGSGFWGQGTLVTVSSGGSEGKSSGSGSESKSTGGSDIVLTQSPDSLAVSLGERATINCKASESVEYFGTSLMHWYQQKPGQPPKLLIYAASNRESGVPDRFSGSGSGTDFTLTISSVQAEDVAVYYCQQTRKVPYTFGQGTKLEIKTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 471 aa, synthetic construct
  REGION 1..471: note = Description of Artificial Sequence: Synthetic polypeptide
DIVLTQSPDSLAVSLGERATINCKASESVEYFGTSLMHWYQQKPGQPPKLLIYAASNRESGVPDRFSGSGSGTDFTLTIQSVQAEDVSVYFCQQTRKVPYTFGQGTKLEIKGGSEGKSSGSGSESKSTGGSQVQLQESGPGLVKPSDTLSLTCAVSGNSITSDYAWNWIRQFPGKGLEWMGYISYSGSTTYNPSLKSRVTISRDTSKNQFSLKLSSVTPVDTAVYYCATGYYYGSGFWGQGTLVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 471 aa, synthetic construct
  REGION 1..471: note = Description of Artificial Sequence: Synthetic polypeptide
DIVLTQSPDSLAVSLGERATINCKASESVEYFGTSLMHWYQQKPGQPPKLLIYAASNRESGVPDRFSGSGSGTDFTLTISSLQAEDVAVYYCQQTRKVPYTFGQGTKLEIKGGSEGKSSGSGSESKSTGGSQVQLQESGPGLVKPSDTLSLTCAVSGNSITSDYAWNWIRQFPGKGLEWIGYISYSGSTTYNPSLKSRVTISRDTSKNQFSLKLSSVTPVDTAVYYCATGYYYGSGFWGQGTLVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 471 aa, synthetic construct
  REGION 1..471: note = Description of Artificial Sequence: Synthetic polypeptide
DIVLTQSPDSLAVSLGERATINCKASESVEYFGTSLMHWYQQKPGQPPKLLIYAASNRESGVPDRFSGSGSGTDFTLTISSLQAEDVAVYYCQQTRKVPYTFGQGTKLEIKGGSEGKSSGSGSESKSTGGSQVQLQESGPGLVKPSDTLSLTCAVSGNSITSDYAWNWIRQFPGKGLEWMGYISYSGSTTYNPSLKSRVTISRDTSKNQFSLKLSSVTPVDTAVYYCATGYYYGSGFWGQGTLVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 471 aa, synthetic construct
  REGION 1..471: note = Description of Artificial Sequence: Synthetic polypeptide
DIVLTQSPDSLAVSLGERATINCKASESVEYFGTSLMHWYQQKPGQPPKLLIYAASNRESGVPDRFSGSGSGTDFTLTISSVQAEDVAVYYCQQTRKVPYTFGQGTKLEIKGGSEGKSSGSGSESKSTGGSQVQLQESGPGLVKPSDTLSLTCAVSGNSITSDYAWNWIRQFPGKGLEWIGYISYSGSTTYNPSLKSRVTISRDTSKNQFSLKLSSVTPVDTAVYYCATGYYYGSGFWGQGTLVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 471 aa, synthetic construct
  REGION 1..471: note = Description of Artificial Sequence: Synthetic polypeptide
DIVLTQSPDSLAVSLGERATINCKASESVEYFGTSLMHWYQQKPGQPPKLLIYAASNRESGVPDRFSGSGSGTDFTLTISSVQAEDVAVYYCQQTRKVPYTFGQGTKLEIKGGSEGKSSGSGSESKSTGGSQVQLQESGPGLVKPSDTLSLTCAVSGNSITSDYAWNWIRQFPGKGLEWMGYISYSGSTTYNPSLKSRVTISRDTSKNQFSLKLSSVTPVDTAVYYCATGYYYGSGFWGQGTLVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 471 aa, synthetic construct
  REGION 1..471: note = Description of Artificial Sequence: Synthetic polypeptide
DIVLTQSPDSLAVSLGERATINCKASESVEYFGTSLMHWYQQKPGQPPKLLIYAASNRESGVPDRFSGSGSGTDFTLTIQSVQAEDVSVYFCQQTRKVPYTFGQGTKLEIKGGSEGKSSGSGSESKSTGGSQVQLQESGPGLVKPSDTLSLTCAVSGNSITSDYAWNWIRQFPGKGLEWIGYISYSGSTTYNPSLKSRVTISRDTSKNQFSLKLSSVTPVDTAVYYCATGYYYGSGFWGQGTLVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 471 aa, synthetic construct
  REGION 1..471: note = Description of Artificial Sequence: Synthetic polypeptide
QVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSSGGSEGKSSGSGSESKSTGGSDIVLTQSPDSLAVSLGERATINCKASESVEYFGTSLMHWYQQKPGQPPKLLIYAASNRESGVPDRFSGSGSGTDFTLTISSVQAEDVAVYYCQQTRKVPYTFGQGTKLEIKTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - protein, 471 aa, synthetic construct
  REGION 1..471: note = Description of Artificial Sequence: Synthetic polypeptide
QVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSSGGSEGKSSGSGSESKSTGGSDIVLTQSPDSLAVSLGERATINCKASESVEYFGTSLMHWYQQKPGQPPKLLIYAASNRESGVPDRFSGSGSGTDFTLTIQSVQAEDVSVYFCQQTRKVPYTFGQGTKLEIKTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 471 aa, synthetic construct
  REGION 1..471: note = Description of Artificial Sequence: Synthetic polypeptide
QVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSSGGSEGKSSGSGSESKSTGGSDIVLTQSPDSLAVSLGERATINCKASESVEYFGTSLMHWYQQKPGQPPKLLIYAASNRESGVPDRFSGSGSGTDFTLTISSLQAEDVAVYYCQQTRKVPYTFGQGTKLEIKTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 471 aa, synthetic construct
  REGION 1..471: note = Description of Artificial Sequence: Synthetic polypeptide
DIVLTQSPDSLAVSLGERATINCKASESVEYFGTSLMHWYQQKPGQPPKLLIYAASNRESGVPDRFSGSGSGTDFTLTIQSVQAEDVSVYFCQQTRKVPYTFGQGTKLEIKGGSEGKSSGSGSESKSTGGSQVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 471 aa, synthetic construct
  REGION 1..471: note = Description of Artificial Sequence: Synthetic polypeptide
DIVLTQSPDSLAVSLGERATINCKASESVEYFGTSLMHWYQQKPGQPPKLLIYAASNRESGVPDRFSGSGSGTDFTLTISSLQAEDVAVYYCQQTRKVPYTFGQGTKLEIKGGSEGKSSGSGSESKSTGGSQVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 471 aa, synthetic construct
  REGION 1..471: note = Description of Artificial Sequence: Synthetic polypeptide
DIVLTQSPDSLAVSLGERATINCKASESVEYFGTSLMHWYQQKPGQPPKLLIYAASNRESGVPDRFSGSGSGTDFTLTISSVQAEDVAVYYCQQTRKVPYTFGQGTKLEIKGGSEGKSSGSGSESKSTGGSQVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 261 aa, Homo sapiens
MWDLVLSIALSVGCTGAVPLIQSRIVGGWECEKHSQPWQVAVYSHGWAHCGGVLVHPQWVLTAAHCLKKNSQVWLGRHNLFEPEDTGQRVPVSHSFPHPLYNMSLLKHQSLRPDEDSSHDLMLLRLSEPAKITDVVKVLGLPTQEPALGTTCYASGWGSIEPEEFLRPRSLQCVSLHLLSNDMCARAYSEKVTEFMLCAGLWTGGKDTCGGDSGGPLVCNGVLQGITSWGPEPCALPEKPAVYTKVVHYRKWIKDTIAANP

SEQ ID NO: 63 - protein, 6 aa, synthetic construct
  REGION 1..6: note = Description of Artificial Sequence: Synthetic peptide
SDYAWN

SEQ ID NO: 64 - protein, 16 aa, synthetic construct
  REGION 1..16: note = Description of Artificial Sequence: Synthetic peptide
YISYSGSTTYSPSLKS

SEQ ID NO: 65 - protein, 16 aa, synthetic construct
  REGION 1..16: note = Description of Artificial Sequence: Synthetic peptide
YISYSGSTTYNPSLKS

SEQ ID NO: 66 - protein, 8 aa, synthetic construct
  REGION 1..8: note = Description of Artificial Sequence: Synthetic peptide
GYYYGSGF

SEQ ID NO: 67 - protein, 15 aa, synthetic construct
  REGION 1..15: note = Description of Artificial Sequence: Synthetic peptide
RASESVEYFGTSLMH

SEQ ID NO: 68 - protein, 15 aa, synthetic construct
  REGION 1..15: note = Description of Artificial Sequence: Synthetic peptide
KASESVEYFGTSLMH

SEQ ID NO: 69 - protein, 7 aa, synthetic construct
  REGION 1..7: note = Description of Artificial Sequence: Synthetic peptide
AASNVES

SEQ ID NO: 70 - protein, 7 aa, synthetic construct
  REGION 1..7: note = Description of Artificial Sequence: Synthetic peptide
AASNRES

SEQ ID NO: 71 - protein, 9 aa, synthetic construct
  REGION 1..9: note = Description of Artificial Sequence: Synthetic peptide
QQTRKVPYT

SEQ ID NO: 72 - protein, 11 aa, synthetic construct
  REGION 1..11: note = Description of Artificial Sequence: Synthetic peptide
GNSITSDYAWN

SEQ ID NO: 73 - protein, 9 aa, synthetic construct
  REGION 1..9: note = Description of Artificial Sequence: Synthetic peptide
YISYSGSTT

SEQ ID NO: 74 - protein, 112 aa, synthetic construct
  REGION 1..112: note = Description of Artificial Sequence: Synthetic polypeptide
  MOD_RES 1: note = D or E
  MOD_RES 9: note = D or A
  MOD_RES 10: note = S or T
  MOD_RES 12: note = A or S
  MOD_RES 13: note = V or L
  MOD_RES 15: note = L or P
  MOD_RES 21: note = I or L
  MOD_RES 22: note = N or S
  MOD_RES 24: note = R or K
  MOD_RES 49: note = K or R
  MOD_RES 58: note = V or R
  MOD_RES 62: note = V or I
  MOD_RES 64: note = A or D
  MOD_RES 80: note = Q or S
  MOD_RES 82: note = L or V
  MOD_RES 83: note = Q or E
  MOD_RES 85: note = P or A
  MOD_RES 88: note = F or V
  MOD_RES 89: note = A or S
  MOD_RES 92: note = Y or F
  MOD_RES 105: note = Q or G
  MOD_RES 109: note = L or V
XIVLTQSPXXLXXSXGERATXXCXASESVEYFGTSLMHWYQQKPGQPPXLLIYAASNXESGXPXRFSGSGSGTDFTLTIXSXXQXEDXXVYXCQQTRKVPYTFGXGTKXEIK

SEQ ID NO: 75 - protein, 117 aa, synthetic construct
  REGION 1..117: note = Description of Artificial Sequence: Synthetic polypeptide
  MOD_RES 16: note = D or Q
  MOD_RES 23: note = A or T
  MOD_RES 41: note = P or F
  MOD_RES 45: note = G or R
  MOD_RES 49: note = I or M
  MOD_RES 70: note = I or M
  MOD_RES 88: note = A or P
  MOD_RES 89: note = V or A
QVQLQESGPGLVKPSXTLSLTCXVSGNSITSDYAWNWIRQXPGKXLEWXGYISYSGSTTYNPSLKSRVTXSRDTSKNQFSLKLSSVTXXDTAVYYCATGYYYGSGFWGQGTLVTVSS

SEQ ID NO: 76 - protein, 8 aa, synthetic construct
  REGION 1..8: note = Description of Artificial Sequence: Synthetic peptide
GGGSGGGS

SEQ ID NO: 77 - protein, 12 aa, synthetic construct
  REGION 1..12: note = Description of Artificial Sequence: Synthetic peptide
GGGSGGGSGGGS

SEQ ID NO: 78 - protein, 16 aa, synthetic construct
  REGION 1..16: note = Description of Artificial Sequence: Synthetic peptide
GGGSGGGSGGGSGGGS

SEQ ID NO: 79 - protein, 20 aa, synthetic construct
  REGION 1..20: note = Description of Artificial Sequence: Synthetic peptide
GGGSGGGSGGGSGGGSGGGS

SEQ ID NO: 80 - protein, 15 aa, synthetic construct
  REGION 1..15: note = Description of Artificial Sequence: Synthetic peptide
GGGGSGGGGSGGGGS

SEQ ID NO: 81 - protein, 20 aa, synthetic construct
  REGION 1..20: note = Description of Artificial Sequence: Synthetic peptide
GGGGSGGGGSGGGGSGGGGS

SEQ ID NO: 82 - protein, 25 aa, synthetic construct
  REGION 1..25: note = Description of Artificial Sequence: Synthetic peptide
GGGGSGGGGSGGGGSGGGGSGGGGS

SEQ ID NO: 83 - protein, 18 aa, synthetic construct
  REGION 1..18: note = Description of Artificial Sequence: Synthetic peptide
GSTSGSGKPGSGEGSTKG

SEQ ID NO: 84 - protein, 14 aa, synthetic construct
  REGION 1..14: note = Description of Artificial Sequence: Synthetic peptide
IRPRAIGGSKPRVA

SEQ ID NO: 85 - protein, 15 aa, synthetic construct
  REGION 1..15: note = Description of Artificial Sequence: Synthetic peptide
GKGGSGKGGSGKGGS

SEQ ID NO: 86 - protein, 15 aa, synthetic construct
  REGION 1..15: note = Description of Artificial Sequence: Synthetic peptide
GGKGSGGKGSGGKGS

SEQ ID NO: 87 - protein, 15 aa, synthetic construct
  REGION 1..15: note = Description of Artificial Sequence: Synthetic peptide
GGGKSGGGKSGGGKS

SEQ ID NO: 88 - protein, 15 aa, synthetic construct
  REGION 1..15: note = Description of Artificial Sequence: Synthetic peptide
GKGKSGKGKSGKGKS

SEQ ID NO: 89 - protein, 15 aa, synthetic construct
  REGION 1..15: note = Description of Artificial Sequence: Synthetic peptide
GGGKSGGKGSGKGGS

SEQ ID NO: 90 - protein, 15 aa, synthetic construct
  REGION 1..15: note = Description of Artificial Sequence: Synthetic peptide
GKPGSGKPGSGKPGS

SEQ ID NO: 91 - protein, 20 aa, synthetic construct
  REGION 1..20: note = Description of Artificial Sequence: Synthetic peptide
GKPGSGKPGSGKPGSGKPGS

SEQ ID NO: 92 - protein, 20 aa, synthetic construct
  REGION 1..20: note = Description of Artificial Sequence: Synthetic peptide
GKGKSGKGKSGKGKSGKGKS

SEQ ID NO: 93 - protein, 14 aa, synthetic construct
  REGION 1..14: note = Description of Artificial Sequence: Synthetic peptide
STAGDTHLGGEDFD

SEQ ID NO: 94 - protein, 15 aa, synthetic construct
  REGION 1..15: note = Description of Artificial Sequence: Synthetic peptide
GEGGSGEGGSGEGGS

SEQ ID NO: 95 - protein, 15 aa, synthetic construct
  REGION 1..15: note = Description of Artificial Sequence: Synthetic peptide
GGEGSGGEGSGGEGS

SEQ ID NO: 96 - protein, 15 aa, synthetic construct
  REGION 1..15: note = Description of Artificial Sequence: Synthetic peptide
GEGESGEGESGEGES

SEQ ID NO: 97 - protein, 15 aa, synthetic construct
  REGION 1..15: note = Description of Artificial Sequence: Synthetic peptide
GGGESGGEGSGEGGS

SEQ ID NO: 98 - protein, 20 aa, synthetic construct
  REGION 1..20: note = Description of Artificial Sequence: Synthetic peptide
GEGESGEGESGEGESGEGES

SEQ ID NO: 99 - protein, 18 aa, synthetic construct
  REGION 1..18: note = Description of Artificial Sequence: Synthetic peptide
GSTSGSGKPGSGEGSTKG

SEQ ID NO: 100 - protein, 19 aa, synthetic construct
  REGION 1..19: note = Description of Artificial Sequence: Synthetic peptide
PRGASKSGSASQTGSAPGS

SEQ ID NO: 101 - protein, 19 aa, synthetic construct
  REGION 1..19: note = Description of Artificial Sequence: Synthetic peptide
GTAAAGAGAAGGAAAGAAG

SEQ ID NO: 102 - protein, 19 aa, synthetic construct
  REGION 1..19: note = Description of Artificial Sequence: Synthetic peptide
GTSGSSGSGSGGSGSGGGG

SEQ ID NO: 103 - protein, 20 aa, synthetic construct
  REGION 1..20: note = Description of Artificial Sequence: Synthetic peptide
GKPGSGKPGSGKPGSGKPGS

SEQ ID NO: 104 - protein, 4 aa, synthetic construct
  REGION 1..4: note = Description of Artificial Sequence: Synthetic peptide
GSGS

SEQ ID NO: 105 - protein, 10 aa, synthetic construct
  REGION 1..10: note = Description of Artificial Sequence: Synthetic peptide
APAPAPAPAP

SEQ ID NO: 106 - protein, 20 aa, synthetic construct
  REGION 1..20: note = Description of Artificial Sequence: Synthetic peptide
APAPAPAPAPAPAPAPAPAP

SEQ ID NO: 107 - protein, 32 aa, synthetic construct
  REGION 1..32: note = Description of Artificial Sequence: Synthetic polypeptide
AEAAAKEAAAKEAAAAKEAAAAKEAAAAKAAA

SEQ ID NO: 108 - protein, 16 aa, synthetic construct
  REGION 1..16: note = Description of Artificial Sequence: Synthetic peptide
GTEGKSSGSGSESKST

SEQ ID NO: 109 - protein, 231 aa, synthetic construct
  REGION 1..231: note = Description of Artificial Sequence: Synthetic polypeptide
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 110 - protein, 231 aa, synthetic construct
  REGION 1..231: note = Description of Artificial Sequence: Synthetic polypeptide
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 111 - protein, 5 aa, Homo sapiens
KVTEF

SEQ ID NO: 112 - protein, 5 aa, Homo sapiens
HYRKW

SEQ ID NO: 113 - protein, 6 aa, Homo sapiens
SHGWAH

SEQ ID NO: 114 - protein, 15 aa, Homo sapiens
RHNLFEPEDTGQRVP

SEQ ID NO: 115 - protein, 9 aa, Homo sapiens
GWGSIEPEE

SEQ ID NO: 116 - protein, 7 aa, synthetic construct
  REGION 1..7: note = Description of Artificial Sequence: Synthetic peptide
NNNAAWS

SEQ ID NO: 117 - protein, 18 aa, synthetic construct
  REGION 1..18: note = Description of Artificial Sequence: Synthetic peptide
RTYYRSKWLYDYAVSVKS

SEQ ID NO: 118 - protein, 8 aa, synthetic construct
  REGION 1..8: note = Description of Artificial Sequence: Synthetic peptide
GYSSSFDY

SEQ ID NO: 119 - protein, 14 aa, synthetic construct
  REGION 1..14: note = Description of Artificial Sequence: Synthetic peptide
TGTSSNIGTYKFVS

SEQ ID NO: 120 - protein, 7 aa, synthetic construct
  REGION 1..7: note = Description of Artificial Sequence: Synthetic peptide
EVSKRPS

SEQ ID NO: 121 - protein, 10 aa, synthetic construct
  REGION 1..10: note = Description of Artificial Sequence: Synthetic peptide
VSYAGSGTLL

SEQ ID NO: 122 - protein, 120 aa, synthetic construct
  REGION 1..120: note = Description of Artificial Sequence: Synthetic polypeptide
QVQLQQSGPRLVRPSQTLSLTCAISGDSVFNNNAAWSWIRQSPSRGLEWLGRTYYRSKWLYDYAVSVKSRITVNPDTSRNQFTLQLNSVTPEDTALYYCARGYSSSFDYWGQGTLVTVSS

SEQ ID NO: 123 - protein, 110 aa, synthetic construct
  REGION 1..110: note = Description of Artificial Sequence: Synthetic polypeptide
QSALTQPASVSGSPGQSITISCTGTSSNIGTYKFVSWYQQHPDKAPKVLLYEVSKRPSGVSSRFSGSKSGNTASLTISGLQAEDQADYHCVSYAGSGTLLFGGGTKLTVL

SEQ ID NO: 124 - protein, 111 aa, synthetic construct
  REGION 1..111: note = Description of Artificial Sequence: Synthetic polypeptide
DIVLTQSPASLAVSLGQRATISCRASESVEYFGTSLMHWYRQKPGQPPKLLIYAASNVESGVPARFSGSGSGTDFSLNIQPVEEDDFSMYFCQQTRKVPYTFGGGTKLEIK

SEQ ID NO: 125 - protein, 117 aa, synthetic construct
  REGION 1..117: note = Description of Artificial Sequence: Synthetic polypeptide
DVQLQESGPGLVKPSQSLSLTCTVTGNSITSDYAWNWIRQFPGNRLEWMGYISYSGSTTYSPSLKSRFSITRDTSKNQFFLQLNSVTPEDTATYFCATGYYYGSGFWGQGTLVTVSS

SEQ ID NO: 126 - protein, 15 aa, synthetic construct
  REGION 1..15: note = Description of Artificial Sequence: Synthetic peptide
EPKSCDKTHTCPPCP

SEQ ID NO: 127 - protein, 12 aa, synthetic construct
  REGION 1..12: note = Description of Artificial Sequence: Synthetic peptide
ERKCCVECPPCP

SEQ ID NO: 128 - protein, 62 aa, synthetic construct
  REGION 1..62: note = Description of Artificial Sequence: Synthetic polypeptide
ELKTPLGDTTHTCPRCPEPKSCDTPPPCPRCPEPKSCDTPPPCPRCPEPKSCDTPPPCPRCP

SEQ ID NO: 129 - protein, 12 aa, synthetic construct
  REGION 1..12: note = Description of Artificial Sequence: Synthetic peptide
ESKYGPPCPSCP

SEQ ID NO: 130 - protein, 330 aa, unidentified [sequence omitted]

SEQ ID NO: 131 - protein, 326 aa, unidentified
  REGION 1..326: note = Description of Unknown: wild-type IgG2 sequence
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 132 - protein, 327 aa, unidentified
  REGION 1..327: note = Description of Unknown: wild-type IgG4 sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 133 - protein, 251 aa, synthetic construct
  REGION 1..251: note = Description of Artificial Sequence: Synthetic polypeptide
EVQLVESGGGLVQPGGSLRLSCAASGFTFSSYWMTWVRQAPGKGLEWVANIKQDGSERYYVDSVKGRFTISRDNAKNSLYLQMNSLRAEDTAVYYCARDQNYDILTGHYGMDVWGQGTTVTVSSGGSEGKSSGSGSESKSTGGSEIVLTQSPSFLSASVGDRVTITCRASQGISSYLSWYQQKPGKAPKLLIYATSTLQSGVPSRFSGSGSGTEFTLTISSLQPEDFATYYCQQLNSYPRTFGQGTKVEIK

SEQ ID NO: 134 - protein, 251 aa, synthetic construct
  REGION 1..251: note = Description of Artificial Sequence: Synthetic polypeptide
EIVLTQSPSFLSASVGDRVTITCRASQGISSYLSWYQQKPGKAPKLLIYATSTLQSGVPSRFSGSGSGTEFTLTISSLQPEDFATYYCQQLNSYPRTFGQGTKVEIKGGSEGKSSGSGSESKSTGGSEVQLVESGGGLVQPGGSLRLSCAASGFTFSSYWMTWVRQAPGKGLEWVANIKQDGSERYYVDSVKGRFTISRDNAKNSLYLQMNSLRAEDTAVYYCARDQNYDILTGHYGMDVWGQGTTVTVSS

SEQ ID NO: 135 - protein, 248 aa, synthetic construct
  REGION 1..248: note = Description of Artificial Sequence: Synthetic polypeptide
QVQLQESGPGLVKPSQTLSLTCTVSGNSITSDYAWNWIRQFPGKRLEWIGYISYSGSTTYNPSLKSRVTISRDTSKNQFSLKLSSVTAADTAVYYCATGYYYGSGFWGQGTLVTVSSGGSEGKSSGSGSESKSTGGSEIVLTQSPATLSLSPGERATLSCRASESVEYFGTSLMHWYQQKPGQPPRLLIYAASNVESGIPARFSGSGSGTDFTLTISSVEPEDFAVYFCQQTRKVPYTFGGGTKVEIK

SEQ ID NO: 136 - protein, 248 aa, synthetic construct
  REGION 1..248: note = Description of Artificial Sequence: Synthetic polypeptide
EIVLTQSPATLSLSPGERATLSCRASESVEYFGTSLMHWYQQKPGQPPRLLIYAASNVESGIPARFSGSGSGTDFTLTISSVEPEDFAVYFCQQTRKVPYTFGGGTKVEIKGGSEGKSSGSGSESKSTGGSQVQLQESGPGLVKPSQTLSLTCTVSGNSITSDYAWNWIRQFPGKRLEWIGYISYSGSTTYNPSLKSRVTISRDTSKNQFSLKLSSVTAADTAVYYCATGYYYGSGFWGQGTLVTVSS

SEQ ID NO: 137 - protein, 124 aa, synthetic construct
  REGION 1..124: note = Description of Artificial Sequence: Synthetic polypeptide
EVQLVESGGGLVQPGGSLRLSCAASGFTFSSYWMTWVRQAPGKGLEWVANIKQDGSERYYVDSVKGRFTISRDNAKNSLYLQMNSLRAEDTAVYYCARDQNYDILTGHYGMDVWGQGTTVTVSS

SEQ ID NO: 138 - protein, 107 aa, synthetic construct
  REGION 1..107: note = Description of Artificial Sequence: Synthetic polypeptide
EIVLTQSPSFLSASVGDRVTITCRASQGISSYLSWYQQKPGKAPKLLIYATSTLQSGVPSRFSGSGSGTEFTLTISSLQPEDFATYYCQQLNSYPRTFGQGTKVEIK

SEQ ID NO: 139 - protein, 117 aa, synthetic construct
  REGION 1..117: note = Description of Artificial Sequence: Synthetic polypeptide
QVQLQESGPGLVKPSQTLSLTCTVSGNSITSDYAWNWIRQFPGKRLEWIGYISYSGSTTYNPSLKSRVTISRDTSKNQFSLKLSSVTAADTAVYYCATGYYYGSGFWGQGTLVTVSS

SEQ ID NO: 140 - protein, 111 aa, synthetic construct
  REGION 1..111: note = Description of Artificial Sequence: Synthetic polypeptide
EIVLTQSPATLSLSPGERATLSCRASESVEYFGTSLMHWYQQKPGQPPRLLIYAASNVESGIPARFSGSGSGTDFTLTISSVEPEDFAVYFCQQTRKVPYTFGGGTKVEIK

SEQ ID NO: 141 - protein, 5 aa, synthetic construct
  REGION 1..5: note = Description of Artificial Sequence: Synthetic peptide
SYWMT

SEQ ID NO: 142 - protein, 17 aa, synthetic construct
  REGION 1..17: note = Description of Artificial Sequence: Synthetic peptide
NIKQDGSERYYVDSVKG

SEQ ID NO: 143 - protein, 15 aa, synthetic construct
  REGION 1..15: note = Description of Artificial Sequence: Synthetic peptide
DQNYDILTGHYGMDV

SEQ ID NO: 144 - protein, 11 aa, synthetic construct
  REGION 1..11: note = Description of Artificial Sequence: Synthetic peptide
RASQGISSYLS

SEQ ID NO: 145 - protein, 7 aa, synthetic construct
  REGION 1..7: note = Description of Artificial Sequence: Synthetic peptide
ATSTLQS

SEQ ID NO: 146 - protein, 9 aa, synthetic construct
  REGION 1..9: note = Description of Artificial Sequence: Synthetic peptide
QQLNSYPRT

SEQ ID NO: 147 - protein, 10 aa, synthetic construct
  REGION 1..10: note = Description of Artificial Sequence: Synthetic peptide
GFTFSSYWMT

SEQ ID NO: 148 - protein, 10 aa, synthetic construct
  REGION 1..10: note = Description of Artificial Sequence: Synthetic peptide
NIKQDGSERY

SEQ ID NO: 149 - protein, 270 aa, synthetic construct
  REGION 1..270: note = Description of Artificial Sequence: Synthetic polypeptide
MAWVWTLLFLMAAAQSIQAEVQLVESGGGLVQPGGSLRLSCAASGFTFSSYWMTWVRQAPGKGLEWVANIKQDGSERYYVDSVKGRFTISRDNAKNSLYLQMNSLRAEDTAVYYCARDQNYDILTGHYGMDVWGQGTTVTVSSGGSEGKSSGSGSESKSTGGSEIVLTQSPSFLSASVGDRVTITCRASQGISSYLSWYQQKPGKAPKLLIYATSTLQSGVPSRFSGSGSGTEFTLTISSLQPEDFATYYCQQLNSYPRTFGQGTKVEIK

SEQ ID NO: 150 - protein, 270 aa, synthetic construct
  REGION 1..270: note = Description of Artificial Sequence: Synthetic polypeptide
MAWVWTLLFLMAAAQSIQAEIVLTQSPSFLSASVGDRVTITCRASQGISSYLSWYQQKPGKAPKLLIYATSTLQSGVPSRFSGSGSGTEFTLTISSLQPEDFATYYCQQLNSYPRTFGQGTKVEIKGGSEGKSSGSGSESKSTGGSEVQLVESGGGLVQPGGSLRLSCAASGFTFSSYWMTWVRQAPGKGLEWVANIKQDGSERYYVDSVKGRFTISRDNAKNSLYLQMNSLRAEDTAVYYCARDQNYDILTGHYGMDVWGQGTTVTVSS

SEQ ID NO: 151 - protein, 267 aa, synthetic construct
  REGION 1..267: note = Description of Artificial Sequence: Synthetic polypeptide
MAWVWTLLFLMAAAQSIQAQVQLQESGPGLVKPSQTLSLTCTVSGNSITSDYAWNWIRQFPGKRLEWIGYISYSGSTTYNPSLKSRVTISRDTSKNQFSLKLSSVTAADTAVYYCATGYYYGSGFWGQGTLVTVSSGGSEGKSSGSGSESKSTGGSEIVLTQSPATLSLSPGERATLSCRASESVEYFGTSLMHWYQQKPGQPPRLLIYAASNVESGIPARFSGSGSGTDFTLTISSVEPEDFAVYFCQQTRKVPYTFGGGTKVEIK

SEQ ID NO: 152 - protein, 267 aa, synthetic construct
  REGION 1..267: note = Description of Artificial Sequence: Synthetic polypeptide
MAWVWTLLFLMAAAQSIQAEIVLTQSPATLSLSPGERATLSCRASESVEYFGTSLMHWYQQKPGQPPRLLIYAASNVESGIPARFSGSGSGTDFTLTISSVEPEDFAVYFCQQTRKVPYTFGGGTKVEIKGGSEGKSSGSGSESKSTGGSQVQLQESGPGLVKPSQTLSLTCTVSGNSITSDYAWNWIRQFPGKRLEWIGYISYSGSTTYNPSLKSRVTISRDTSKNQFSLKLSSVTAADTAVYYCATGYYYGSGFWGQGTLVTVSS

SEQ ID NO: 153 - protein, 474 aa, synthetic construct
  REGION 1..474: note = Description of Artificial Sequence: Synthetic polypeptide
EVQLVESGGGLVQPGGSLRLSCAASGFTFSSYWMTWVRQAPGKGLEWVANIKQDGSERYYVDSVKGRFTISRDNAKNSLYLQMNSLRAEDTAVYYCARDQNYDILTGHYGMDVWGQGTTVTVSSGGSEGKSSGSGSESKSTGGSEIVLTQSPSFLSASVGDRVTITCRASQGISSYLSWYQQKPGKAPKLLIYATSTLQSGVPSRFSGSGSGTEFTLTISSLQPEDFATYYCQQLNSYPRTFGQGTKVEIKTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 154 - protein, 474 aa, synthetic construct
  REGION 1..474: note = Description of Artificial Sequence: Synthetic polypeptide
EIVLTQSPSFLSASVGDRVTITCRASQGISSYLSWYQQKPGKAPKLLIYATSTLQSGVPSRFSGSGSGTEFTLTISSLQPEDFATYYCQQLNSYPRTFGQGTKVEIKGGSEGKSSGSGSESKSTGGSEVQLVESGGGLVQPGGSLRLSCAASGFTFSSYWMTWVRQAPGKGLEWVANIKQDGSERYYVDSVKGRFTISRDNAKNSLYLQMNSLRAEDTAVYYCARDQNYDILTGHYGMDVWGQGTTVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 155 - protein, 471 aa, synthetic construct
  REGION 1..471: note = Description of Artificial Sequence: Synthetic polypeptide
QVQLQESGPGLVKPSQTLSLTCTVSGNSITSDYAWNWIRQFPGKRLEWIGYISYSGSTTYNPSLKSRVTISRDTSKNQFSLKLSSVTAADTAVYYCATGYYYGSGFWGQGTLVTVSSGGSEGKSSGSGSESKSTGGSEIVLTQSPATLSLSPGERATLSCRASESVEYFGTSLMHWYQQKPGQPPRLLIYAASNVESGIPARFSGSGSGTDFTLTISSVEPEDFAVYFCQQTRKVPYTFGGGTKVEIKTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 156 - protein, 471 aa, synthetic construct
  REGION 1..471: note = Description of Artificial Sequence: Synthetic polypeptide
EIVLTQSPATLSLSPGERATLSCRASESVEYFGTSLMHWYQQKPGQPPRLLIYAASNVESGIPARFSGSGSGTDFTLTISSVEPEDFAVYFCQQTRKVPYTFGGGTKVEIKGGSEGKSSGSGSESKSTGGSQVQLQESGPGLVKPSQTLSLTCTVSGNSITSDYAWNWIRQFPGKRLEWIGYISYSGSTTYNPSLKSRVTISRDTSKNQFSLKLSSVTAADTAVYYCATGYYYGSGFWGQGTLVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 157 - protein, 107 aa, synthetic construct
  REGION 1..107: note = Description of Artificial Sequence: Synthetic polypeptide
  MOD_RES 4: note = L or M
  MOD_RES 9: note = G or S
  MOD_RES 10: note = I or S
  MOD_RES 13: note = V or A
  MOD_RES 15: note = P or V
  MOD_RES 17: note = E or D
  MOD_RES 20: note = S or T
  MOD_RES 21: note = F or I
  MOD_RES 22: note = S or T
  MOD_RES 40: note = T or P
  MOD_RES 41: note = N or G
  MOD_RES 42: note = G or K
  MOD_RES 43: note = S or A
  MOD_RES 45: note = R or K
  MOD_RES 49: note = K or Y
  MOD_RES 58: note = I or V
  MOD_RES 76: note = N or S
  MOD_RES 78: note = V or L
  MOD_RES 80: note = S or P
  MOD_RES 83: note = I or F
  MOD_RES 85: note = D or T
  MOD_RES 92: note = N or G
  MOD_RES 100: note = G or Q
DIQXTQSPXXLSXSXGXRVXXXCRARQSIGTAIHWYQQKXXXXPXLLIXYASESISGXPSRFSGSGSGTDFTLTIXSXQXEDXAXYYCQQSXSWPYTFGXGTKLEIK

SEQ ID NO: 158 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 159 - protein, 117 aa, synthetic construct
  REGION 1..117: note = Description of Artificial Sequence: Synthetic polypeptide
QVQLQESGPGLVKPSQTLSLTCTVSGNSITSDYAWNWIRQFPGKGLEWIGYISYSGSTTYNPSLKSRVTISRDTSKNQFSLKLSSVTAADTAVYYCATGYYYGSGFWGQGTLVTVSS

SEQ ID NO: 160 - protein, 111 aa, synthetic construct
  REGION 1..111: note = Description of Artificial Sequence: Synthetic polypeptide
DIVLTQSPDSLAVSLGERATINCRASESVEYFGTSLMHWYQQKPGQPPKLLIYAASNVESGVPDRFSGSGSGTDFTLTISSLQAEDVAVYFCQQTRKVPYTFGGGTKVEIK

SEQ ID NO: 161 - protein, 117 aa, synthetic construct
  REGION 1..117: note = Description of Artificial Sequence: Synthetic polypeptide
QVQLQESGPGLVKPSQTLSLTCTVSGNSITSDYAWNWIRQPPGKGLEWIGYISYSGSTTYNPSLKSRVTISRDTSKNQFSLKLSSVTAADTAVYYCATGYYYGSGFWGQGTLVTVSS

SEQ ID NO: 162 - protein, 125 aa, synthetic construct
  REGION 1..125: note = Description of Artificial Sequence: Synthetic polypeptide
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSS

SEQ ID NO: 163 - protein, 107 aa, synthetic construct
  REGION 1..107: note = Description of Artificial Sequence: Synthetic polypeptide
DIQMTQSPSFLSASVGDRVTITCRASQGISSYLAWYQQKPGKAPKFLIYAASTLQSGVPSRFSGSGSGTEFTLTISSLQPEDFATYYCQQLNSYPLTFGGGTKVEIK

SEQ ID NO: 164 - protein, 123 aa, synthetic construct
  REGION 1..123: note = Description of Artificial Sequence: Synthetic polypeptide
EVQLVESGGGVVQPGRSLRLSCVASGFTFSSYDIHWVRQAPGKGLEWVAIISYDGSKKDYTDSVKGRFTISRDNSKNTLYLQMDSLRVEDSAVYSCARESGWSHYYYYGMDVWGQGTMVTVSS

SEQ ID NO: 165 - protein, 107 aa, synthetic construct
  REGION 1..107: note = Description of Artificial Sequence: Synthetic polypeptide
DIVMTQSPSSLSASVGDRVTITCRASQDISNYLAWYQQKPGKVPKFLIYAASTLHSGVPSRFSGSGSGTDFTLTISSLQPEDVATYYCQKYNSAPYTFGQGTRLEIK

SEQ ID NO: 166 - protein, 119 aa, synthetic construct
  REGION 1..119: note = Description of Artificial Sequence: Synthetic polypeptide
QVQLQESGPGLVKPSETLSLTCTVSGGSISSYYWSWLRQPAGSGLEWIGRLYVSGFTNYNPSLKSRVTLSLDPSRNQLSLKLSSVTAADTAVYYCAGDSGNYWGWFDPWGQGTLVTVSS

SEQ ID NO: 167 - protein, 106 aa, synthetic construct
  REGION 1..106: note = Description of Artificial Sequence: Synthetic polypeptide
SYELTQPPSVSVSPGETASITCSGDQLGENYACWYQQKPGQSPVLVIYQDSKRPSGIPERFSGSNSGNTATLTISGTQALDEADYYCQAWDNSIVVFGGGTKLTVL

SEQ ID NO: 168 - protein, 121 aa, synthetic construct
  REGION 1..121: note = Description of Artificial Sequence: Synthetic polypeptide
QVQLVESGGGVVQPGRSLRLSCAASGFTFSYYGMHWVRQAPGKGLEWVAFISYDGSNKYYADSVKGRFTISRDNSKNTLYLQMNSLRAEDTAVYYCAHLPYSGSYWAFDYWGQGTQVTVSS

SEQ ID NO: 169 - protein, 108 aa, synthetic construct
  REGION 1..108: note = Description of Artificial Sequence: Synthetic polypeptide
QSVLTQPPSVSVAPGQTASITCGGDNIGSKSVHWYQQKPGQAPVLVVYDNSDRPSGIPERFSGSNSGTTATLTISRVEAGDEADYYCQVWDSSSDHPVVFGGGTKVTV

SEQ ID NO: 170 - protein, 5 aa, synthetic construct
  REGION 1..5: note = Description of Artificial Sequence: Synthetic peptide
SYYWS

SEQ ID NO: 171 - protein, 16 aa, synthetic construct
  REGION 1..16: note = Description of Artificial Sequence: Synthetic peptide
YIYYSGSTNYNPSLKS

SEQ ID NO: 172 - protein, 17 aa, synthetic construct
  REGION 1..17: note = Description of Artificial Sequence: Synthetic peptide
TTIFGVVTPNFYYGMDV

SEQ ID NO: 173 - protein, 11 aa, synthetic construct
  REGION 1..11: note = Description of Artificial Sequence: Synthetic peptide
RASQGISSYLA

SEQ ID NO: 174 - protein, 7 aa, synthetic construct
  REGION 1..7: note = Description of Artificial Sequence: Synthetic peptide
AASTLQS

SEQ ID NO: 175 - protein, 9 aa, synthetic construct
  REGION 1..9: note = Description of Artificial Sequence: Synthetic peptide
QQLNSYPLT

SEQ ID NO: 176 - protein, 5 aa, synthetic construct
  REGION 1..5: note = Description of Artificial Sequence: Synthetic peptide
SYDIH

SEQ ID NO: 177 - protein, 17 aa, synthetic construct
  REGION 1..17: note = Description of Artificial Sequence: Synthetic peptide
IISYDGSKKDYTDSVKG

SEQ ID NO: 178 - protein, 14 aa, synthetic construct
  REGION 1..14: note = Description of Artificial Sequence: Synthetic peptide
ESGWSHYYYYGMDV

SEQ ID NO: 179 - protein, 11 aa, synthetic construct
  REGION 1..11: note = Description of Artificial Sequence: Synthetic peptide
RASQDISNYLA

SEQ ID NO: 180 - protein, 7 aa, synthetic construct
  REGION 1..7: note = Description of Artificial Sequence: Synthetic peptide
AASTLHS

SEQ ID NO: 181 - protein, 9 aa, synthetic construct
  REGION 1..9: note = Description of Artificial Sequence: Synthetic peptide
QKYNSAPYT

SEQ ID NO: 182 - protein, 11 aa, synthetic construct
  REGION 1..11: note = Description of Artificial Sequence: Synthetic peptide
GGNNIGSKSVH

SEQ ID NO: 183 - protein, 16 aa, synthetic construct
  REGION 1..16: note = Description of Artificial Sequence: Synthetic peptide
RLYVSGFTNYNPSLKS

SEQ ID NO: 184 - protein, 11 aa, synthetic construct
  REGION 1..11: note = Description of Artificial Sequence: Synthetic peptide
DSGNYWGWFDP

SEQ ID NO: 185 - protein, 11 aa, synthetic construct
  REGION 1..11: note = Description of Artificial Sequence: Synthetic peptide
SGDQLGENYAC

SEQ ID NO: 186 - protein, 7 aa, synthetic construct
  REGION 1..7: note = Description of Artificial Sequence: Synthetic peptide
QDSKRPS

SEQ ID NO: 187 - protein, 9 aa, synthetic construct
  REGION 1..9: note = Description of Artificial Sequence: Synthetic peptide
QAWDNSIVV

SEQ ID NO: 188 - protein, 5 aa, synthetic construct
  REGION 1..5: note = Description of Artificial Sequence: Synthetic peptide
YYGMH

SEQ ID NO: 189 - protein, 17 aa, synthetic construct
  REGION 1..17: note = Description of Artificial Sequence: Synthetic peptide
FISYDGSNKYYADSVKG

SEQ ID NO: 190 - protein, 12 aa, synthetic construct
  REGION 1..12: note = Description of Artificial Sequence: Synthetic peptide
LPYSGSYWAFDY

SEQ ID NO: 191 - protein, 11 aa, synthetic construct
  REGION 1..11: note = Description of Artificial Sequence: Synthetic peptide
GGDNIGSKSVH

SEQ ID NO: 192 - protein, 7 aa, synthetic construct
  REGION 1..7: note = Description of Artificial Sequence: Synthetic peptide
DNSDRPS

SEQ ID NO: 193 - protein, 12 aa, synthetic construct
  REGION 1..12: note = Description of Artificial Sequence: Synthetic peptide
QVWDSSSDHPVV

SEQ ID NO: 194 - protein, 10 aa, synthetic construct
  REGION 1..10: note = Description of Artificial Sequence: Synthetic peptide
GGSISSYYWS

SEQ ID NO: 195 - protein, 9 aa, synthetic construct
  REGION 1..9: note = Description of Artificial Sequence: Synthetic peptide
YIYYSGSTN

SEQ ID NO: 196 - protein, 10 aa, synthetic construct
  REGION 1..10: note = Description of Artificial Sequence: Synthetic peptide
GFTFSSYDIH

SEQ ID NO: 197 - protein, 10 aa, synthetic construct
  REGION 1..10: note = Description of Artificial Sequence: Synthetic peptide
IISYDGSKKD

SEQ ID NO: 198 - protein, 10 aa, synthetic construct
  REGION 1..10: note = Description of Artificial Sequence: Synthetic peptide
GGSISSYYWS

SEQ ID NO: 199 - protein, 9 aa, synthetic construct
  REGION 1..9: note = Description of Artificial Sequence: Synthetic peptide
RLYVSGFTN

SEQ ID NO: 200 - protein, 7 aa, synthetic construct
  REGION 1..7: note = Description of Artificial Sequence: Synthetic peptide
GFTFSYY

SEQ ID NO: 201 - protein, 10 aa, synthetic construct
  REGION 1..10: note = Description of Artificial Sequence: Synthetic peptide
FISYDGSNKY

SEQ ID NO: 202 - protein, 447 aa, synthetic construct
  REGION 1..447: note = Description of Artificial Sequence: Synthetic polypeptide
DVQLQESGPGLVKPSQSLSLTCTVTGNSITSDYAWNWIRQFPGNRLEWMGYISYSGSTTYSPSLKSRFSITRDTSKNQFFLQLNSVTPEDTATYFCATGYYYGSGFWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 203 - protein, 447 aa, synthetic construct
  REGION 1..447: note = Description of Artificial Sequence: Synthetic polypeptide
QVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 204 - protein, 125 aa, synthetic construct
  REGION 1..125: note = Description of Artificial Sequence: Synthetic polypeptide
QVQLVESGGGLVQPGGSLRLSCAASGFTFSHYAMSWVRQAPGKGLEWVSTIGGSGGSTYYADSVKGRFTISRDNSKNTLYLQMNSLRAEDTAVYYCAKPHIVMVTALLYDGMDVWGQGTMVTVSS

SEQ ID NO: 205 - protein, 108 aa, synthetic construct
  REGION 1..108: note = Description of Artificial Sequence: Synthetic polypeptide
SSELTQPPSVSVAPGQTARITCGGNNIGSKSVHWYQQKPGQAPVLVVYDDSDRPSGIPERFSGSNSGNTATLTISRVEAGDEADYYCQVWDSSSDHVVFGGGTKLTVL

SEQ ID NO: 206 - protein, 5 aa, synthetic construct
  REGION 1..5: note = Description of Artificial Sequence: Synthetic peptide
HYAMS

SEQ ID NO: 207 - protein, 17 aa, synthetic construct
  REGION 1..17: note = Description of Artificial Sequence: Synthetic peptide
TIGGSGGSTYYADSVKG

SEQ ID NO: 208 - protein, 16 aa, synthetic construct
  REGION 1..16: note = Description of Artificial Sequence: Synthetic peptide
PHIVMVTALLYDGMDV

SEQ ID NO: 209 - protein, 11 aa, synthetic construct
  REGION 1..11: note = Description of Artificial Sequence: Synthetic peptide
QVWDSSSDHVV

SEQ ID NO: 210 - protein, 452 aa, synthetic construct
  REGION 1..452: note = Description of Artificial Sequence: Synthetic polypeptide
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 211 - protein, 450 aa, synthetic construct
  REGION 1..450: note = Description of Artificial Sequence: Synthetic polypeptide
EVQLVESGGGVVQPGRSLRLSCVASGFTFSSYDIHWVRQAPGKGLEWVAIISYDGSKKDYTDSVKGRFTISRDNSKNTLYLQMDSLRVEDSAVYSCARESGWSHYYYYGMDVWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 212 - protein, 446 aa, synthetic construct
  REGION 1..446: note = Description of Artificial Sequence: Synthetic polypeptide
QVQLQESGPGLVKPSETLSLTCTVSGGSISSYYWSWLRQPAGSGLEWIGRLYVSGFTNYNPSLKSRVTLSLDPSRNQLSLKLSSVTAADTAVYYCAGDSGNYWGWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 213 - protein, 451 aa, synthetic construct
  REGION 1..451: note = Description of Artificial Sequence: Synthetic polypeptide
QVQLVESGGGVVQPGRSLRLSCAASGFTFSYYGMHWVRQAPGKGLEWVAFISYDGSNKYYADSVKGRFTISRDNSKNTLYLQMNSLRAEDTAVYYCAHLPYSGSYWAFDYWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 214 - protein, 218 aa, synthetic construct
  REGION 1..218: note = Description of Artificial Sequence: Synthetic polypeptide
DIVLTQSPASLAVSLGQRATISCRASESVEYFGTSLMHWYRQKPGQPPKLLIYAASNVESGVPARFSGSGSGTDFSLNIQPVEEDDFSMYFCQQTRKVPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 215 - protein, 218 aa, synthetic construct
  REGION 1..218: note = Description of Artificial Sequence: Synthetic polypeptide
DIVLTQSPDSLAVSLGERATINCKASESVEYFGTSLMHWYQQKPGQPPKLLIYAASNRESGVPDRFSGSGSGTDFTLTISSLQAEDVAVYYCQQTRKVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 216 - protein, 10 aa, synthetic construct
  REGION 1..10: note = Description of Artificial Sequence: Synthetic peptide
GFTFSHYAMS

SEQ ID NO: 217 - protein, 11 aa, synthetic construct
  REGION 1..11: note = Description of Artificial Sequence: Synthetic peptide
TIGGSGGSTYY

SEQ ID NO: 218 - protein, 16 aa, synthetic construct
  REGION 1..16: note = Description of Artificial Sequence: Synthetic peptide
PHIVMVTALLYDGMDV

SEQ ID NO: 219 - protein, 455 aa, synthetic construct
  REGION 1..455: note = Description of Artificial Sequence: Synthetic polypeptide
QVQLVESGGGLVQPGGSLRLSCAASGFTFSHYAMSWVRQAPGKGLEWVSTIGGSGGSTYYADSVKGRFTISRDNSKNTLYLQMNSLRAEDTAVYYCAKPHIVMVTALLYDGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 220 - protein, 214 aa, synthetic construct
  REGION 1..214: note = Description of Artificial Sequence: Synthetic polypeptide
SSELTQPPSVSVAPGQTARITCGGNNIGSKSVHWYQQKPGQAPVLVVYDDSDRPSGIPERFSGSNSGNTATLTISRVEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 221 - protein, 214 aa, synthetic construct
  REGION 1..214: note = Description of Artificial Sequence: Synthetic polypeptide
DIQMTQSPSFLSASVGDRVTITCRASQGISSYLAWYQQKPGKAPKFLIYAASTLQSGVPSRFSGSGSGTEFTLTISSLQPEDFATYYCQQLNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 222 - protein, 214 aa, synthetic construct
  REGION 1..214: note = Description of Artificial Sequence: Synthetic polypeptide
DIVMTQSPSSLSASVGDRVTITCRASQDISNYLAWYQQKPGKVPKFLIYAASTLHSGVPSRFSGSGSGTDFTLTISSLQPEDVATYYCQKYNSAPYTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 223 - protein, 212 aa, synthetic construct
  REGION 1..212: note = Description of Artificial Sequence: Synthetic polypeptide
SYELTQPPSVSVSPGETASITCSGDQLGENYACWYQQKPGQSPVLVIYQDSKRPSGIPERFSGSNSGNTATLTISGTQALDEADYYCQAWDNSIVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 224 - protein, 215 aa, synthetic construct
  REGION 1..215: note = Description of Artificial Sequence: Synthetic polypeptide
QSVLTQPPSVSVAPGQTASITCGGDNIGSKSVHWYQQKPGQAPVLVVYDNSDRPSGIPERFSGSNSGTTATLTISRVEAGDEADYYCQVWDSSSDHPVVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 225 - DNA, 351 bp, synthetic construct
  misc_feature 1..351: note = Description of Artificial Sequence: Synthetic polynucleotide
  source 1..351: mol_type = other DNA
gatgtgcagcttcaggagtctggacccggacttgttaaaccaagtcagtctctgtccctgacctgtaccgtcaccggcaacagcatcacaagcgattacgcatggaactggatcaggcagttccctggaaatcgactcgaatggatgggctacatttcatactccggttcaaccacttactctccatccttgaaatctaggttcagcatcacccgtgatacctcaaagaaccaattttttctgcaactgaatagcgtaactccagaggacacagccacatatttctgcgccactgggtattactatggctcaggtttctggggtcagggcactctcgtcaccgtcagcagc

SEQ ID NO: 226 - DNA, 351 bp, synthetic construct
  misc_feature 1..351: note = Description of Artificial Sequence: Synthetic polynucleotide
  source 1..351: mol_type = other DNA
caggtccaactgcaagagagcggaccgggcctggtaaagccatccgacacattgtccctgacgtgtgcggtaagtggaaactctatcactagcgactatgcgtggaattggataagacaaccgccgggcaaggggctggaatggataggatatatcagctattccggttctacgacatacaatccttccctgaaaagcagagtcactatgtcacgcgacacgtccaagaatcagttctcattgaaattgtcatccgtaacggccgttgacactgcggtttattattgcgcaaccggatattactacggctctggtttttggggacagggaacacttgttactgttagttca

SEQ ID NO: 227 - DNA, 351 bp, synthetic construct
  misc_feature 1..351: note = Description of Artificial Sequence: Synthetic polynucleotide
  source 1..351: mol_type = other DNA
caggtgcagctgcaggagagcggcccaggcctggtgaagccaagcgacaccctgagcctgacctgcgccgtgagcggcaacagcatcaccagcgactacgcctggaactggatccgccagttcccaggcaagggcctggagtggatcggctacatcagctacagcggcagcaccacctacaacccaagcctgaagagccgcgtcaccatcagccgcgacaccagcaagaaccagttcagcctgaagctgagcagcgtgacccctgtggacaccgccgtgtactactgcgccaccggctactactacggcagcggcttctggggccagggcaccctggtgaccgtgagcagc

SEQ ID NO: 228 - DNA, 351 bp, synthetic construct
  misc_feature 1..351: note = Description of Artificial Sequence: Synthetic polynucleotide
  source 1..351: mol_type = other DNA
caggtgcagctgcaggagagcggcccaggcctggtgaagccaagcgacaccctgagcctgacctgcgccgtgagcggcaacagcatcaccagcgactacgcctggaactggatccgccagttcccaggcaagggcctggagtggatgggctacatcagctacagcggcagcaccacctacaacccaagcctgaagagccgcgtcaccatcagccgcgacaccagcaagaaccagttcagcctgaagctgagcagcgtgacccctgtggacaccgccgtgtactactgcgccaccggctactactacggcagcggcttctggggccagggcaccctggtgaccgtgagcagc

SEQ ID NO: 229 - DNA, 351 bp, synthetic construct
  misc_feature 1..351: note = Description of Artificial Sequence: Synthetic polynucleotide
  source 1..351: mol_type = other DNA
caggttcagctgcaagagtctggaccaggcctggtcaagccctctcagaccctgtctctgacctgtaccgtgtccggcaactccatcacctctgactacgcctggaactggattcggcagttccctggcaagggccttgagtggatcggctacatctcctactccggttccaccacctacaaccccagcctgaagtcccgggtcaccatctcccgcgacacctccaagaaccagttctccctgaagctgtcctccgtgaccgctgctgataccgccgtgtactactgtgccaccggctactactacggctccggcttttggggacagggcacactggttaccgtgtctagt

SEQ ID NO: 230 - DNA, 351 bp, synthetic construct
  misc_feature 1..351: note = Description of Artificial Sequence: Synthetic polynucleotide
  source 1..351: mol_type = other DNA
caggttcagctgcaagagtctggaccaggcctggtcaagccctctcagaccctgtctctgacctgtaccgtgtccggcaactccatcacctctgactacgcctggaactggattcggcagccacctggcaagggccttgagtggatcggctacatctcctactccggttccaccacctacaaccccagcctgaagtcccgggtcaccatctcccgcgacacctccaagaaccagttctccctgaagctgtcctccgtgaccgctgctgataccgccgtgtactactgtgccaccggctactactacggctccggcttttggggacagggcacactggttaccgtgtctagt

SEQ ID NO: 231 - DNA, 351 bp, synthetic construct
  misc_feature 1..351: note = Description of Artificial Sequence: Synthetic polynucleotide
  source 1..351: mol_type = other DNA
caggttcagctgcaagagtctggaccaggcctggtcaagccctctcagaccctgtctctgacctgtaccgtgtccggcaactccatcacctctgactacgcctggaactggattcggcagttccctggcaagcgccttgagtggatcggctacatctcctactccggttccaccacctacaaccccagcctgaagtcccgggtcaccatctcccgcgacacctccaagaaccagttctccctgaagctgtcctccgtgaccgctgctgataccgccgtgtactactgtgccaccggctactactacggctccggcttttggggacagggcacactggttaccgtgtctagt

SEQ ID NO: 232 - DNA, 372 bp, synthetic construct
  misc_feature 1..372: note = Description of Artificial Sequence: Synthetic polynucleotide
  source 1..372: mol_type = other DNA
gaggtgcaacttgtggagagcggcggaggtctggtccaacccggaggaagtctccgtctctcctgtgctgctagtggcttcactttcagctcatattggatgacatgggtgagacaagccccaggaaaggggctcgagtgggtagctaacattaaacaggacggctccgaacggtactatgttgattctgtgaagggacggttcactatatccagggataatgcaaaaaattcactctatcttcaaatgaactcactcagagcagaggacactgccgtgtattattgcgccagggatcaaaattatgacatactgaccggtcattatggaatggatgtttggggccagggaacaaccgttaccgtctcaagt

SEQ ID NO: 233 - DNA, 375 bp, synthetic construct
  misc_feature 1..375: note = Description of Artificial Sequence: Synthetic polynucleotide
  source 1..375: mol_type = other DNA
caggtgcagctgcaggagtcgggcccaggactggtgaagccttcggagaccctgtccctcacctgcactgtctctggtggctccatcagtagttactactggagctggatccggcagcccccagggaagggactggagtggattggatatatctattacagtgggagcaccaactacaacccctccctcaagagtcgagtcaccatatcagtagacacgtccaagaaccagttctccctgaagctgagctctgtgaccgctgcggacacggccgtgtattactgtgcggggactacgatttttggagtggttacccccaacttctactacggtatggacgtctggggccaagggaccacggtcaccgtctcctca

SEQ ID NO: 234 - DNA, 369 bp, synthetic construct
  misc_feature 1..369: note = Description of Artificial Sequence: Synthetic polynucleotide
  source 1..369: mol_type = other DNA
gaggtgcagctggtggagtctgggggaggcgtggtccagcctgggaggtccctgagactctcctgtgtagcctctggattcaccttcagtagttatgacatacactgggtccgccaggctccaggcaaggggctggagtgggtggcaattatttcatatgatggaagtaaaaaagactatacagactccgtgaagggccgattcaccatctccagagacaattccaagaacacgctgtatctgcaaatggacagcctgagagttgaggactcggctgtgtattcctgtgcgagagaaagtggctggtcccactactactattacggtatggacgtctggggccaagggacaatggtcaccgtctcttca

SEQ ID NO: 235 - DNA, 357 bp, synthetic construct
  misc_feature 1..357: note = Description of Artificial Sequence: Synthetic polynucleotide
  source 1..357: mol_type = other DNA
caggtgcagctgcaggagtcgggcccaggactggtgaagccttcggagaccctgtccctcacctgcactgtctctggtggctccatcagtagttactattggagctggctccggcagcccgccgggtcgggactggagtggattgggcgtttatatgtcagtgggttcaccaactacaacccctccctcaagagtcgagtcaccttgtcactagacccgtccaggaaccagttgtccctgaaactgagttctgtgaccgccgcggacacggccgtatattattgtgcgggagatagtgggaactactggggttggttcgacccctggggccagggaaccctggtcaccgtctcctca

SEQ ID NO: 236 - DNA, 363 bp, synthetic construct
  misc_feature 1..363: note = Description of Artificial Sequence: Synthetic polynucleotide
  source 1..363: mol_type = other DNA
caggtgcagctggtggagtctgggggaggcgtggtccagcctgggaggtccctgagactctcctgtgcagcctctggattcaccttcagttactatggcatgcactgggtccgccaggctccaggcaaggggctggagtgggtggcatttatatcatatgatggaagtaataaatactatgcagactccgtgaagggccgattcaccatctccagagacaattccaagaacacgctgtatctgcaaatgaacagcctgagagctgaggacacggctgtgtattactgtgcccacctcccttatagtgggagctactgggcctttgactactggggccagggaacccaggtcaccgtctcttca

SEQ ID NO: 237 - DNA, 333 bp, synthetic construct
  misc_feature 1..333: note = Description of Artificial Sequence: Synthetic polynucleotide
  source 1..333: mol_type = other DNA
gacattgtgctgacacagagtccagcatccttggcagtatctttggggcagcgggcaacaatttcatgccgtgcatctgaaagtgtggagtattttggaacttctcttatgcactggtatcgccagaagcctgggcagcctcccaaactccttatatatgccgcttccaacgtggagtccggagtaccagcacgcttttccggctctgggtccggcacagacttttccctcaatatccaacctgttgaagaagacgatttttccatgtatttttgccaacagacacgcaaggttccatatacattcggcggcggcactaaacttgagatcaaa

SEQ ID NO: 238 - DNA, 333 bp, synthetic construct
  misc_feature 1..333: note = Description of Artificial Sequence: Synthetic polynucleotide
  source 1..333: mol_type = other DNA
gacatagtcttgactcagagcccggattcccttgctgtgtctctgggagaacgagctacgatcaactgcaaggcaagtgaatccgtagaatacttcgggacatcattgatgcattggtatcaacagaaaccggggcaaccgcccaaattgctgatatatgcggctagtaatagagaatcaggagtaccggataggtttagtggttcaggatcaggtacagatttcaccctgacaataagtagcttgcaagccgaagacgtagcagtgtattactgccaacaaacccgaaaggtgccatatacgtttggacagggtacaaagttggaaatcaaa

SEQ ID NO: 239 - DNA, 333 bp, synthetic construct
  misc_feature 1..333: note = Description of Artificial Sequence: Synthetic polynucleotide
  source 1..333: mol_type = other DNA
gacatcgtgctgacccagagcccagacagcctggccgtgagcctgggcgagcgcgccaccatcaactgcaaggccagcgagagcgtggagtacttcggcaccagcctgatgcactggtaccagcagaagccaggccagccaccaaagctgctgatctacgctgccagcaaccgcgagagcggcgtgccagaccgcttcagcggcagcggcagcggcaccgacttcaccctgaccatccagagcgtgcaggccgaggacgtctccgtgtacttctgccagcagacccgcaaggtgccatacaccttcggccagggcaccaagctggagatcaag

SEQ ID NO: 240 - DNA, 333 bp, synthetic construct
  misc_feature 1..333: note = Description of Artificial Sequence: Synthetic polynucleotide
  source 1..333: mol_type = other DNA
gacatcgtgctgacccagagcccagacagcctggccgtgagcctgggcgagcgcgccaccatcaactgcaaggccagcgagagcgtggagtacttcggcaccagcctgatgcactggtaccagcagaagccaggccagccaccaaagctgctgatctacgctgccagcaaccgcgagagcggcgtgccagaccgcttcagcggcagcggcagcggcaccgacttcaccctgaccatcagcagcgtgcaggccgaggacgtcgccgtgtactactgccagcagacccgcaaggtgccatacaccttcggccagggcaccaagctggagatcaag

SEQ ID NO: 241 - DNA, 333 bp, synthetic construct
  misc_feature 1..333: note = Description of Artificial Sequence: Synthetic polynucleotide
  source 1..333: mol_type = other DNA
gacatcgtgctgacccagtctccagactctctggctgtgtctctgggcgagagagccaccatcaactgcagagcctccgagtccgtggaatacttcggcacctctctgatgcactggtaccagcagaagcccggccagcctcctaagctgctgatctacgccgcctccaacgtggaatctggcgtgcccgatagattttccggctctggctctggcaccgactttaccctgaccatcagctctctgcaggccgaggatgtggccgtgtacttctgtcagcagacccggaaggtgccctacacatttggcggcggaacaaaggtggaaatcaag

SEQ ID NO: 242 - DNA, 333 bp, synthetic construct
  misc_feature 1..333: note = Description of Artificial Sequence: Synthetic polynucleotide
  source 1..333: mol_type = other DNA
gagatcgtgctgacccagtctcctgccacactgtcactgtctccaggcgagagagccaccctctcttgtagagcctccgagtccgtggaatacttcggcacctctctgatgcactggtaccagcagaagcccggccagcctcctagactgctgatctacgccgcctccaacgtcgaatctggcatccccgctagattctccggctctggctctggcacagactttaccctgaccatctcctccgtggaacccgaggatttcgctgtgtacttttgccagcagacccggaaggtgccctacacatttggcggcggaacaaaggtggaaatcaag

SEQ ID NO: 243 - DNA, 321 bp, synthetic construct
  misc_feature 1..321: note = Description of Artificial Sequence: Synthetic polynucleotide
  source 1..321: mol_type = other DNA
gaaatcgtactgacccagtccccttctttcttgagtgcatcagttggggatagagtgaccattacttgtagagcatctcaaggtatttcttcatacttgtcttggtatcaacaaaaacctggcaaggcacccaaactcttgatctacgccacctctacattgcaaagtggggttccttctaggttttcaggctccggctctggtaccgagttcaccctcactataagcagtctccaacctgaagatttcgctacttattattgtcagcagcttaattcttatccccgaacctttggtcaaggaactaaggtcgagatcaaa

SEQ ID NO: 244 - DNA, 321 bp, synthetic construct
  misc_feature 1..321: note = Description of Artificial Sequence: Synthetic polynucleotide
  source 1..321: mol_type = other DNA
gacatccagatgacccagtctccttccttcctgtctgcatctgtaggagacagagtcaccatcacttgccgggccagtcagggcattagcagttatttagcctggtatcagcaaaaaccagggaaagcccctaagttcctgatctatgctgcatccactttgcaaagtggggtcccatcaaggttcagcggcagtggatctgggacagaattcactctcacaatcagcagcctgcagcctgaagattttgcaacttattactgtcaacagcttaatagttaccctctcactttcggcggagggaccaaggtggaaatcaaa

SEQ ID NO: 245 - DNA, 321 bp, synthetic construct
  misc_feature 1..321: note = Description of Artificial Sequence: Synthetic polynucleotide
  source 1..321: mol_type = other DNA
gacatcgtgatgacccagtctccatcctccctgtctgcatctgtaggagacagagtcaccatcacttgccgggcgagtcaggacattagcaattatttagcctggtatcagcagaaaccagggaaagttcctaagttcctgatctatgctgcatccactttgcactctggggtcccatctcggttcagtggcagtggatctgggacagatttcactctcaccatcagcagcctgcagcctgaagatgttgcaacttattactgtcaaaagtataacagtgccccgtacacttttggccaagggacacgactggagattaaa

SEQ ID NO: 246 - DNA, 318 bp, synthetic construct
  misc_feature 1..318: note = Description of Artificial Sequence: Synthetic polynucleotide
  source 1..318: mol_type = other DNA
tcctatgagctgactcagccaccctcagtgtccgtgtccccaggagagacagccagcatcacctgctctggagatcaattgggggaaaattatgcttgctggtatcagcagaagccaggccagtcccctgtgttggtcatctatcaagatagtaagcggccctcagggatccctgagcgattctctggctccaactctgggaacacagccactctgaccatcagcgggacccaggctctggatgaggctgactattactgtcaggcgtgggacaacagtattgtggtattcggcggagggaccaagctgaccgtccta

SEQ ID NO: 247 - DNA, 327 bp, synthetic construct
  misc_feature 1..327: note = Description of Artificial Sequence: Synthetic polynucleotide
  source 1..327: mol_type = other DNA
cagtctgtgctgactcagccaccctcggtgtcagtggcccccgggcagacggccagtattacctgtgggggagacaacattggaagtaaaagtgtgcactggtaccagcagaagccaggccaggcccctgtgctggtcgtctatgataatagcgaccggccctcagggatccctgagcgattctctggctccaactctgggaccacggccaccctgaccatcagcagggtcgaagccggggatgaggccgactattactgtcaggtgtgggatagtagtagtgatcatcctgtggtattcggcggagggaccaaggtcaccgtccta

SEQ ID NO: 248 - protein, 116 aa, synthetic construct
  REGION 1..116: note = Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSS

SEQ ID NO: 249 - protein, 107 aa, synthetic construct
  REGION 1..107: note = Description of Artificial Sequence: Synthetic polypeptide
DILLTQSPGILSVSPGERVSFSCRARQSIGTAIHWYQQRTNGSPRLLIKYASESISGIPSRFSGSGSGTDFTLTINSVESEDIADYYCQQSNSWPYTFGGGTKLEIK

SEQ ID NO: 250 - protein, 107 aa, synthetic construct
  REGION 1..107: note = Description of Artificial Sequence: Synthetic polypeptide
DIQMTQSPSSLSASVGDRVTITCRARQSIGTAIHWYQQKPGKAPKLLIYYASESISGVPSRFSGSGSGTDFTLTISSVQPEDFATYYCQQSGSWPYTFGQGTKLEIK

SEQ ID NO: 251 - protein, 107 aa, synthetic construct
  REGION 1..107: note = Description of Artificial Sequence: Synthetic polypeptide
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIK

SEQ ID NO: 252 - protein, 107 aa, synthetic construct
  REGION 1..107: note = Description of Artificial Sequence: Synthetic polypeptide
DIQMTQSPSSLSASVGDRVTITCRARQSIGTAIHWYQQKPGKAPKLLIKYASESISGVPSRFSGSGSGTDFTLTISSVQPEDFATYYCQQSGSWPYTFGQGTKLEIK

SEQ ID NO: 253 - protein, 107 aa, synthetic construct
  REGION 1..107: note = Description of Artificial Sequence: Synthetic polypeptide
DIQMTQSPSSLSASVGDRVTITCRARQSIGTAIHWYQQKPGKAPKLLIYYASESISGVPSRFSGSGSGTDFTLTISSLQPEDFATYYCQQSGSWPYTFGQGTKLEIK

SEQ ID NO: 254 - protein, 107 aa, synthetic construct
  REGION 1..107: note = Description of Artificial Sequence: Synthetic polypeptide
DILLTQSPGILSVSPGERVSFSCRARQSIGTAIHWYQQRTNGSPRLLIKYASESISGIPSRFSGSGSGTDFTLTINSVESEDIADYYCQQSGSWPYTFGGGTKLEIK

SEQ ID NO: 255 - protein, 5 aa, synthetic construct
  REGION 1..5: note = Description of Artificial Sequence: Synthetic peptide
RYNMN

SEQ ID NO: 256 - protein, 17 aa, synthetic construct
  REGION 1..17: note = Description of Artificial Sequence: Synthetic peptide
SISTSSNYIYYADSVKG

SEQ ID NO: 257 - protein, 7 aa, synthetic construct
  REGION 1..7: note = Description of Artificial Sequence: Synthetic peptide
GWGPFDY

SEQ ID NO: 258 - protein, 11 aa, synthetic construct
  REGION 1..11: note = Description of Artificial Sequence: Synthetic peptide
RARQSIGTAIH

SEQ ID NO: 259 - protein, 7 aa, synthetic construct
  REGION 1..7: note = Description of Artificial Sequence: Synthetic peptide
YASESIS

SEQ ID NO: 260 - protein, 9 aa, synthetic construct
  REGION 1..9: note = Description of Artificial Sequence: Synthetic peptide
QQSNSWPYT

SEQ ID NO: 261 - protein, 9 aa, synthetic construct
  REGION 1..9: note = Description of Artificial Sequence: Synthetic peptide
QQSGSWPYT

SEQ ID NO: 262 - protein, 446 aa, synthetic construct
  REGION 1..446: note = Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 263 - DNA, 375 bp, synthetic construct
  misc_feature 1..375: note = Description of Artificial Sequence: Synthetic polynucleotide
  source 1..375: mol_type = other DNA
caggtgcagctggtggagtctgggggaggcttggtacagcctggggggtccctgagactctcctgtgcagcctctggattcacctttagtcattatgccatgagctgggtccgccaggctccagggaaggggctggagtgggtctcaactattggtggtagtggtggtagcacatactacgcagactccgtgaagggccggttcaccatctccagagacaattccaagaacacgctgtatctgcaaatgaacagcctgagagccgaggacacggccgtatattactgtgcgaaacctcatattgtaatggtgactgctcttctctacgacggtatggacgtctggggccaagggacaatggtcaccgtctcctca

SEQ ID NO: 264 - protein, 446 aa, synthetic construct
  REGION 1..446: note = Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 265 - protein, 214 aa, synthetic construct
  REGION 1..214: note = Description of Artificial Sequence: Synthetic polypeptide
DILLTQSPGILSVSPGERVSFSCRARQSIGTAIHWYQQRTNGSPRLLIKYASESISGIPSRFSGSGSGTDFTLTINSVESEDIADYYCQQSNSWPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 266 - protein, 214 aa, synthetic construct
  REGION 1..214: note = Description of Artificial Sequence: Synthetic polypeptide
DIQMTQSPSSLSASVGDRVTITCRARQSIGTAIHWYQQKPGKAPKLLIYYASESISGVPSRFSGSGSGTDFTLTISSVQPEDFATYYCQQSGSWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 267 - protein, 214 aa, synthetic construct
  REGION 1..214: note = Description of Artificial Sequence: Synthetic polypeptide
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 268 - protein, 214 aa, synthetic construct
  REGION 1..214: note = Description of Artificial Sequence: Synthetic polypeptide
DIQMTQSPSSLSASVGDRVTITCRARQSIGTAIHWYQQKPGKAPKLLIKYASESISGVPSRFSGSGSGTDFTLTISSVQPEDFATYYCQQSGSWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 269 - protein, 214 aa, synthetic construct
  REGION 1..214: note = Description of Artificial Sequence: Synthetic polypeptide
DIQMTQSPSSLSASVGDRVTITCRARQSIGTAIHWYQQKPGKAPKLLIYYASESISGVPSRFSGSGSGTDFTLTISSLQPEDFATYYCQQSGSWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 270 - protein, 214 aa, synthetic construct
  REGION 1..214: note = Description of Artificial Sequence: Synthetic polypeptide
DILLTQSPGILSVSPGERVSFSCRARQSIGTAIHWYQQRTNGSPRLLIKYASESISGIPSRFSGSGSGTDFTLTINSVESEDIADYYCQQSGSWPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 271 - DNA, 324 bp, synthetic construct
  misc_feature 1..324: note = Description of Artificial Sequence: Synthetic polynucleotide
  source 1..324: mol_type = other DNA
tcttctgagctgactcagccaccctcggtgtcagtggccccaggacagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcatgtggtattcggcggagggaccaagctgaccgtccta

SEQ ID NO: 272 - protein, 216 aa, synthetic construct
  REGION 1..216: note = Description of Artificial Sequence: Synthetic polypeptide
QSALTQPASVSGSPGQSITISCTGTSSNIGTYKFVSWYQQHPDKAPKVLLYEVSKRPSGVSSRFSGSKSGNTASLTISGLQAEDQADYHCVSYAGSGTLLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 273 - DNA, 348 bp, synthetic construct
  misc_feature 1..348: note = Description of Artificial Sequence: Synthetic polynucleotide
  source 1..348: mol_type = other DNA
gaggtgcaactggtggagtctgggggaggcctggtcaagcctggggggtccctgagactctcctgtgcagcctctggattcaccttcagtagatataacatgaactgggtccgccaggctccagggaaggggctggagtgggtctcatccattagtactagtagtaattacatatactacgcagactcagtgaagggccgattcaccttctccagagacaacgccaagaactcactggatctgcaaatgagcggcctgagagccgaggacacggctatttattactgtacgagaggctgggggccttttgactactggggccagggaaccctggtcaccgtctcctca

SEQ ID NO: 274 - DNA, 1365 bp, synthetic construct
  misc_feature 1..1365: note = Description of Artificial Sequence: Synthetic polynucleotide
  source 1..1365: mol_type = other DNA
caggtgcagctggtggagtctgggggaggcttggtacagcctggggggtccctgagactctcctgtgcagcctctggattcacctttagtcattatgccatgagctgggtccgccaggctccagggaaggggctggagtgggtctcaactattggtggtagtggtggtagcacatactacgcagactccgtgaagggccggttcaccatctccagagacaattccaagaacacgctgtatctgcaaatgaacagcctgagagccgaggacacggccgtatattactgtgcgaaacctcatattgtaatggtgactgctcttctctacgacggtatggacgtctggggccaagggacaatggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagagcctctccctgtctccgggtaaa

SEQ ID NO: 275 - DNA, 360 bp, synthetic construct
  misc_feature 1..360: note = Description of Artificial Sequence: Synthetic polynucleotide
  source 1..360: mol_type = other DNA
caggtgcagctgcagcagtctggccctagactcgtgcggccttcccagaccctgtctctgacctgtgccatctccggcgactccgtgttcaacaacaacgccgcctggtcctggatccggcagagcccttctagaggcctggaatggctgggccggacctactaccggtccaagtggctgtacgactacgccgtgtccgtgaagtcccggatcaccgtgaaccctgacacctcccggaaccagttcaccctgcagctgaactccgtgacccctgaggacaccgccctgtactactgcgccagaggctactcctcctccttcgactattggggccagggcaccctcgtgaccgtgtcctct

SEQ ID NO: 276 - DNA, 321 bp, synthetic construct
  misc_feature 1..321: note = Description of Artificial Sequence: Synthetic polynucleotide
  source 1..321: mol_type = other DNA
gatatacttcttacccagagtcccggcatcctctccgttagccctggggagagagtctcattctcatgccgagccagacagtcaattggtaccgcaatacactggtatcaacagcggaccaatggttctccccgacttctgataaagtacgcatcagaatcaattagtggaataccatcaagatttagtggctcagggagtggaaccgattttactctgaccatcaactcagtggaatctgaggacattgccgactactactgtcaacaaagcaatagttggccatataccttcggaggcggaactaaattggagataaaa

SEQ ID NO: 277 - DNA, 321 bp, synthetic construct
  misc_feature 1..321: note = Description of Artificial Sequence: Synthetic polynucleotide
  source 1..321: mol_type = other DNA
gacatccagatgacacagtcaccttctagtttgtctgcttctgtaggcgaccgtgtaactatcacctgtcgagcccgtcaaagtattggtactgccattcactggtaccaacaaaaacctggcaaagctccaaaactcttgatctactatgcctccgaaagcatatcaggggtcccaagcagattctcaggcagtggcagtggcactgacttcactctcaccatttctagcgtgcaaccagaggacttcgccacttattactgccaacagtcagggagctggccctacaccttcggccaaggtacaaaactggagatcaaa

SEQ ID NO: 278 - DNA, 321 bp, synthetic construct
  misc_feature 1..321: note = Description of Artificial Sequence: Synthetic polynucleotide
  source 1..321: mol_type = other DNA
gacatacaaatgacacaatcaccctcttctctttctgcaagcgttggcgaccgtgtcactatcacttgtcgagcccgccagtccataggtactgccattcactggtatcaacagaagcctggcaaggctcccaaactcctgattaagtatgccagcgagagcatttccggcgtaccttcaagattttccggctccggtagtgggacagatttcactctcactatatctagcctccaaccagaagatttcgccacttactactgtcaacaatcaggttcatggccttacactttcggccaggggacaaaattggagatcaag

SEQ ID NO: 279 - DNA, 321 bp, synthetic construct
  misc_feature 1..321: note = Description of Artificial Sequence: Synthetic polynucleotide
  source 1..321: mol_type = other DNA
gacatccaaatgactcaatcacctagcagcctctccgcctccgttggagatagagtgacaataacttgccgagcccggcaaagtatcggaactgctattcactggtatcaacaaaaacctggaaaggcacctaagctcttgattaaatacgcttctgagtccatctccggcgtgccttcacgattcagcggcagcggtagtggtactgactttaccctcactattagttctgttcagccagaggacttcgcaacttattactgccaacagagtggttcctggccatacacttttggccaggggactaaattggaaatcaaa

SEQ ID NO: 280 - DNA, 321 bp, synthetic construct
  misc_feature 1..321: note = Description of Artificial Sequence: Synthetic polynucleotide
  source 1..321: mol_type = other DNA
gacatccaaatgactcaaagcccctctagtttgagtgcatctgtaggtgaccgggtaacaatcacctgccgtgcccggcaaagtataggtactgcaatccactggtaccagcaaaaacccggcaaagcaccaaagctgctcatatactatgctagtgagagcatttctggcgttcctagtcgattttctggatcagggagtggaactgattttacactgacaatcagcagcctccaacccgaagacttcgccacctactattgtcagcagtctgggtcctggccttacacattcggtcaaggaactaaattggagatcaaa

SEQ ID NO: 281 - DNA, 321 bp, synthetic construct
  misc_feature 1..321: note = Description of Artificial Sequence: Synthetic polynucleotide
  source 1..321: mol_type = other DNA
gacattttgctgacacagagccctggtatcctctcagtcagtccaggggaacgcgtttcatttagctgccgtgctcgacagagcattgggaccgcaatccactggtaccaacaaagaactaacggttcaccacggcttttgattaagtatgcctccgaatccatcagtggcattcctagtcgtttttctggatcaggatcaggcaccgactttactctcacaattaatagtgtcgaaagtgaggacattgcagactattattgtcagcaatccggttcctggccctatacttttggtggtggtactaagttggaaattaaa

SEQ ID NO: 282 - DNA, 642 bp, synthetic construct
  misc_feature 1..642: note = Description of Artificial Sequence: Synthetic polynucleotide
  source 1..642: mol_type = other DNA
tcttctgagctgactcagccaccctcggtgtcagtggccccaggacagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcatgtggtattcggcggagggaccaagctgaccgtcctaggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 283 - DNA, 329 bp, synthetic construct
  misc_feature 1..329: note = Description of Artificial Sequence: Synthetic polynucleotide
  source 1..329: mol_type = other DNA
agtctgctctgacccagcctgcctccgtgtctggctctcccggccagtccatcaccatcagctgtaccggcacctcctccaacatcggcacctacaagttcgtgtcctggtatcagcagcaccccgacaaggcccccaaagtgctgctgtacgaggtgtccaagcggccctctggcgtgtcctccagattctccggctccaagtctggcaacaccgcctccctgaccatcagcggactgcaggctgaggaccaggccgactaccactgtgtgtcctacgctggctctggcaccctgctgtttggcggaggcaccaagctgaccgtgctg

SEQ ID NO: 284 - DNA, 1341 bp, synthetic construct
  misc_feature 1..1341: note = Description of Artificial Sequence: Synthetic polynucleotide
  source 1..1341: mol_type = other DNA
gatgtgcagcttcaggagtctggacccggacttgttaaaccaagtcagtctctgtccctgacctgtaccgtcaccggcaacagcatcacaagcgattacgcatggaactggatcaggcagttccctggaaatcgactcgaatggatgggctacatttcatactccggttcaaccacttactctccatccttgaaatctaggttcagcatcacccgtgatacctcaaagaaccaattttttctgcaactgaatagcgtaactccagaggacacagccacatatttctgcgccactgggtattactatggctcaggtttctggggtcagggcactctcgtcaccgtcagcagcgccaaaacaacagcaccaagtgtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcaccgacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 285 - DNA, 1341 bp, synthetic construct
  misc_feature 1..1341: note = Description of Artificial Sequence: Synthetic polynucleotide
  source 1..1341: mol_type = other DNA
caggtccaactgcaagagagcggaccgggcctggtaaagccatccgacacattgtccctgacgtgtgcggtaagtggaaactctatcactagcgactatgcgtggaattggataagacaaccgccgggcaaggggctggaatggataggatatatcagctattccggttctacgacatacaatccttccctgaaaagcagagtcactatgtcacgcgacacgtccaagaatcagttctcattgaaattgtcatccgtaacggccgttgacactgcggtttattattgcgcaaccggatattactacggctctggtttttggggacagggaacacttgttactgttagttcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagagcctctccctgtctccgggtaaa

SEQ ID NO: 286-291 - skipped (6 entries)

SEQ ID NO: 292 - DNA, 1356 bp, synthetic construct
  misc_feature 1..1356: note = Description of Artificial Sequence: Synthetic polynucleotide
  source 1..1356: mol_type = other DNA
caggtgcagctgcaggagtcgggcccaggactggtgaagccttcggagaccctgtccctcacctgcactgtctctggtggctccatcagtagttactactggagctggatccggcagcccccagggaagggactggagtggattggatatatctattacagtgggagcaccaactacaacccctccctcaagagtcgagtcaccatatcagtagacacgtccaagaaccagttctccctgaagctgagctctgtgaccgctgcggacacggccgtgtattactgtgcggggactacgatttttggagtggttacccccaacttctactacggtatggacgtctggggccaagggaccacggtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaaaacctacacttgcaacgtagatcacaagcccagcaacaccaaggtggacaagagagttgagtccaaatatggtcccccatgcccaccatgcccagcacctgaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcagatggcaggaggggaatgtcttctcatgctccgtgatgcatgaggctctgcacaaccactacacacagaagagcctctccctgtctctgggtaaa

SEQ ID NO: 293 - DNA, 1350 bp, synthetic construct
  misc_feature 1..1350: note = Description of Artificial Sequence: Synthetic polynucleotide
  source 1..1350: mol_type = other DNA
gaggtgcagctggtggagtctgggggaggcgtggtccagcctgggaggtccctgagactctcctgtgtagcctctggattcaccttcagtagttatgacatacactgggtccgccaggctccaggcaaggggctggagtgggtggcaattatttcatatgatggaagtaaaaaagactatacagactccgtgaagggccgattcaccatctccagagacaattccaagaacacgctgtatctgcaaatggacagcctgagagttgaggactcggctgtgtattcctgtgcgagagaaagtggctggtcccactactactattacggtatggacgtctggggccaagggacaatggtcaccgtctcttcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaaaacctacacttgcaacgtagatcacaagcccagcaacaccaaggtggacaagagagttgagtccaaatatggtcccccatgcccaccatgcccagcacctgaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcagatggcaggaggggaatgtcttctcatgctccgtgatgcatgaggctctgcacaaccactacacacagaagagcctctccctgtctctgggtaaa

SEQ ID NO: 294 - DNA, 1338 bp, synthetic construct
  misc_feature 1..1338: note = Description of Artificial Sequence: Synthetic polynucleotide
  source 1..1338: mol_type = other DNA
caggtgcagctgcaggagtcgggcccaggactggtgaagccttcggagaccctgtccctcacctgcactgtctctggtggctccatcagtagttactattggagctggctccggcagcccgccgggtcgggactggagtggattgggcgtttatatgtcagtgggttcaccaactacaacccctccctcaagagtcgagtcaccttgtcactagacccgtccaggaaccagttgtccctgaaactgagttctgtgaccgccgcggacacggccgtatattattgtgcgggagatagtgggaactactggggttggttcgacccctggggccagggaaccctggtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaaaacctacacttgcaacgtagatcacaagcccagcaacaccaaggtggacaagagagttgagtccaaatatggtcccccatgcccaccatgcccagcacctgaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcagatggcaggaggggaatgtcttctcatgctccgtgatgcatgaggctctgcacaaccactacacacagaagagcctctccctgtctctgggtaaa

SEQ ID NO: 295 - DNA, 1353 bp, synthetic construct
  misc_feature 1..1353: note = Description of Artificial Sequence: Synthetic polynucleotide
  source 1..1353: mol_type = other DNA
caggtgcagctggtggagtctgggggaggcgtggtccagcctgggaggtccctgagactctcctgtgcagcctctggattcaccttcagttactatggcatgcactgggtccgccaggctccaggcaaggggctggagtgggtggcatttatatcatatgatggaagtaataaatactatgcagactccgtgaagggccgattcaccatctccagagacaattccaagaacacgctgtatctgcaaatgaacagcctgagagctgaggacacggctgtgtattactgtgcccacctcccttatagtgggagctactgggcctttgactactggggccagggaacccaggtcaccgtctcttcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagagcctctccctgtctccgggtaaa

SEQ ID NO: 296 - DNA, 654 bp, synthetic construct
  misc_feature 1..654: note = Description of Artificial Sequence: Synthetic polynucleotide
  source 1..654: mol_type = other DNA
gacattgtgctgacacagagtccagcatccttggcagtatctttggggcagcgggcaacaatttcatgccgtgcatctgaaagtgtggagtattttggaacttctcttatgcactggtatcgccagaagcctgggcagcctcccaaactccttatatatgccgcttccaacgtggagtccggagtaccagcacgcttttccggctctgggtccggcacagacttttccctcaatatccaacctgttgaagaagacgatttttccatgtatttttgccaacagacacgcaaggttccatatacattcggcggcggcactaaacttgagatcaaacgggctgatgctgcaccgactgtgtccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 297 - DNA, 654 bp, synthetic construct
  misc_feature 1..654: note = Description of Artificial Sequence: Synthetic polynucleotide
  source 1..654: mol_type = other DNA
gacatagtcttgactcagagcccggattcccttgctgtgtctctgggagaacgagctacgatcaactgcaaggcaagtgaatccgtagaatacttcgggacatcattgatgcattggtatcaacagaaaccggggcaaccgcccaaattgctgatatatgcggctagtaatagagaatcaggagtaccggataggtttagtggttcaggatcaggtacagatttcaccctgacaataagtagcttgcaagccgaagacgtagcagtgtattactgccaacaaacccgaaaggtgccatatacgtttggacagggtacaaagttggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 298-302 - skipped (5 entries)

SEQ ID NO: 303 - DNA, 642 bp, synthetic construct
  misc_feature 1..642: note = Description of Artificial Sequence: Synthetic polynucleotide
  source 1..642: mol_type = other DNA
gatattcaaatgacccagtcaccatcattcctgtccgcctcagtgggagatcgcgtcactattacttgtcgtgctagccaggggatatcatcatatttggcttggtatcaacaaaagccaggaaaggccccaaaattccttatatatgcagctagtacactccagagtggtgttcctagccggttctctggcagcggctcagggaccgagttcaccctgacaatctccagcttgcagcccgaagactttgcaacctactattgccagcaactgaactcctatcctctgactttcgggggaggaaccaaggttgagattaaacggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgt

SEQ ID NO: 304 - DNA, 642 bp, synthetic construct
  misc_feature 1..642: note = Description of Artificial Sequence: Synthetic polynucleotide
  source 1..642: mol_type = other DNA
gatattgtaatgactcagtcaccctcttcactgagtgcatcagtaggtgatcgcgttaccatcacttgccgtgccagtcaagacatttcaaattaccttgcatggtaccaacaaaagcccggaaaagtgccaaagtttttgatttatgccgcttcaacactccattcaggagtgccctctcgtttcagtggatctggcagtggcaccgattttactctcacaataagcagtctccagcctgaggatgtagccacctattattgccaaaaatataattcagccccctatacttttggacagggcacacgccttgagattaaacggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgt

SEQ ID NO: 305 - DNA, 636 bp, synthetic construct
  misc_feature 1..636: note = Description of Artificial Sequence: Synthetic polynucleotide
  source 1..636: mol_type = other DNA
tcctatgagctgactcagccaccctcagtgtccgtgtccccaggagagacagccagcatcacctgctctggagatcaattgggggaaaattatgcttgctggtatcagcagaagccaggccagtcccctgtgttggtcatctatcaagatagtaagcggccctcagggatccctgagcgattctctggctccaactctgggaacacagccactctgaccatcagcgggacccaggctctggatgaggctgactattactgtcaggcgtgggacaacagtattgtggtattcggcggagggaccaagctgaccgtcctaggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 306 - DNA, 645 bp, synthetic construct
  misc_feature 1..645: note = Description of Artificial Sequence: Synthetic polynucleotide
  source 1..645: mol_type = other DNA
cagtctgtgctgactcagccaccctcggtgtcagtggcccccgggcagacggccagtattacctgtgggggagacaacattggaagtaaaagtgtgcactggtaccagcagaagccaggccaggcccctgtgctggtcgtctatgataatagcgaccggccctcagggatccctgagcgattctctggctccaactctgggaccacggccaccctgaccatcagcagggtcgaagccggggatgaggccgactattactgtcaggtgtgggatagtagtagtgatcatcctgtggtattcggcggagggaccaaggtcaccgtcctaggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 307 - skipped

SEQ ID NO: 308 - protein, 253 aa, synthetic construct
  REGION 1..253: note = Description of Artificial Sequence: Synthetic polypeptide
QVQLVESGGGLVQPGGSLRLSCAASGFTFSHYAMSWVRQAPGKGLEWVSTIGGSGGSTYYADSVKGRFTISRDNSKNTLYLQMNSLRAEDTAVYYCAKPHIVMVTALLYDGMDVWGQGTMVTVSSGGSEGKSSGSGSESKSTGGSSSELTQPPSVSVAPGQTARITCGGNNIGSKSVHWYQQKPGQAPVLVVYDDSDRPSGIPERFSGSNSGNTATLTISRVEAGDEADYYCQVWDSSSDHVVFGGGTKLTVL

SEQ ID NO: 309 - protein, 107 aa, Homo sapiens [sequence omitted]

SEQ ID NO: 310 - skipped

SEQ ID NO: 311 - DNA, 642 bp, synthetic construct
  misc_feature 1..642: note = Description of Artificial Sequence: Synthetic polynucleotide
  source 1..642: mol_type = other DNA
gacatccagatgacccagagcccaagcagcctgagcgccagcgtcggcgaccgcgtgaccatcacctgtcgtgcccgccagagcatcggcaccgccatccactggtaccagcagaagccaggcaaggccccaaagctgctgatctactacgccagcgagagcatcagcggtgtgccaagccgcttcagcggcagcggcagcggcaccgacttcaccctgaccatcagcagcgtgcagccagaggacttcgccacctactactgccagcagagcggcagctggccatacaccttcggccagggcaccaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 312 - DNA, 642 bp, synthetic construct
  misc_feature 1..642: note = Description of Artificial Sequence: Synthetic polynucleotide
  source 1..642: mol_type = other DNA
gacatacaaatgacacaatcaccctcttctctttctgcaagcgttggcgaccgtgtcactatcacttgtcgagcccgccagtccataggtactgccattcactggtatcaacagaagcctggcaaggctcccaaactcctgattaagtatgccagcgagagcatttccggcgtaccttcaagattttccggctccggtagtgggacagatttcactctcactatatctagcctccaaccagaagatttcgccacttactactgtcaacaatcaggttcatggccttacactttcggccaggggacaaaattggagatcaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgt

SEQ ID NO: 313 - DNA, 642 bp, synthetic construct
  misc_feature 1..642: note = Description of Artificial Sequence: Synthetic polynucleotide
  source 1..642: mol_type = other DNA
gacatccagatgacccagagcccaagcagcctgagcgccagcgtcggcgaccgcgtgaccatcacctgtcgtgcccgccagagcatcggcaccgccatccactggtaccagcagaagccaggcaaggccccaaagctgctgatcaagtacgccagcgagagcatcagcggtgtgccaagccgcttcagcggcagcggcagcggcaccgacttcaccctgaccatcagcagcgtgcagccagaggacttcgccacctactactgccagcagagcggcagctggccatacaccttcggccagggcaccaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 314 - DNA, 642 bp, synthetic construct
  misc_feature 1..642: note = Description of Artificial Sequence: Synthetic polynucleotide
  source 1..642: mol_type = other DNA
gacatccagatgacccagagcccaagcagcctgagcgccagcgtcggcgaccgcgtgaccatcacctgtcgtgcccgccagagcatcggcaccgccatccactggtaccagcagaagccaggcaaggccccaaagctgctgatctactacgccagcgagagcatcagcggtgtgccaagccgcttcagcggcagcggcagcggcaccgacttcaccctgaccatcagcagcctgcagccagaggacttcgccacctactactgccagcagagcggcagctggccatacaccttcggccagggcaccaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 315 - DNA, 642 bp, synthetic construct
  misc_feature 1..642: note = Description of Artificial Sequence: Synthetic polynucleotide
  source 1..642: mol_type = other DNA
gacatcttgctgactcagtctccaggcatcctgtctgtgagtccaggagaaagagtcagtttctcctgcagggccagacagagcattggcacagccatacactggtatcagcaaagaacaaatggttctccaaggcttctcataaagtatgcttctgagtctatctctgggatcccttccaggtttagcggcagtggatcagggacagattttactcttaccatcaacagtgtggagtctgaagatattgcagattattactgtcaacaaagtgggagctggccgtacacgttcggaggggggaccaagctggaaataaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 316 - protein, 253 aa, synthetic construct
  REGION 1..253: note = Description of Artificial Sequence: Synthetic polypeptide
SSELTQPPSVSVAPGQTARITCGGNNIGSKSVHWYQQKPGQAPVLVVYDDSDRPSGIPERFSGSNSGNTATLTISRVEAGDEADYYCQVWDSSSDHVVFGGGTKLTVLGGSEGKSSGSGSESKSTGGSQVQLVESGGGLVQPGGSLRLSCAASGFTFSHYAMSWVRQAPGKGLEWVSTIGGSGGSTYYADSVKGRFTISRDNSKNTLYLQMNSLRAEDTAVYYCAKPHIVMVTALLYDGMDVWGQGTMVTVSS

SEQ ID NO: 317 - DNA, 648 bp, synthetic construct
  misc_feature 1..648: note = Description of Artificial Sequence: Synthetic polynucleotide
  source 1..648: mol_type = other DNA
cagtctgctctgacccagcctgcctccgtgtctggctctcccggccagtccatcaccatcagctgtaccggcacctcctccaacatcggcacctacaagttcgtgtcctggtatcagcagcaccccgacaaggcccccaaagtgctgctgtacgaggtgtccaagcggccctctggcgtgtcctccagattctccggctccaagtctggcaacaccgcctccctgaccatcagcggactgcaggctgaggaccaggccgactaccactgtgtgtcctacgctggctctggcaccctgctgtttggcggaggcaccaagctgaccgtgctgggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 318 - protein, 248 aa, synthetic construct
  REGION 1..248: note = Description of Artificial Sequence: Synthetic polypeptide
QVQLQESGPGLVKPSQTLSLTCTVSGNSITSDYAWNWIRQFPGKGLEWIGYISYSGSTTYNPSLKSRVTISRDTSKNQFSLKLSSVTAADTAVYYCATGYYYGSGFWGQGTLVTVSSGGSEGKSSGSGSESKSTGGSDIVLTQSPDSLAVSLGERATINCRASESVEYFGTSLMHWYQQKPGQPPKLLIYAASNVESGVPDRFSGSGSGTDFTLTISSLQAEDVAVYFCQQTRKVPYTFGGGTKVEIK

SEQ ID NO: 319 - protein, 248 aa, synthetic construct
  REGION 1..248: note = Description of Artificial Sequence: Synthetic polypeptide
DIVLTQSPDSLAVSLGERATINCRASESVEYFGTSLMHWYQQKPGQPPKLLIYAASNVESGVPDRFSGSGSGTDFTLTISSLQAEDVAVYFCQQTRKVPYTFGGGTKVEIKGGSEGKSSGSGSESKSTGGSQVQLQESGPGLVKPSQTLSLTCTVSGNSITSDYAWNWIRQFPGKGLEWIGYISYSGSTTYNPSLKSRVTISRDTSKNQFSLKLSSVTAADTAVYYCATGYYYGSGFWGQGTLVTVSS

SEQ ID NO: 320 - protein, 248 aa, synthetic construct
  REGION 1..248: note = Description of Artificial Sequence: Synthetic polypeptide
QVQLQESGPGLVKPSQTLSLTCTVSGNSITSDYAWNWIRQPPGKGLEWIGYISYSGSTTYNPSLKSRVTISRDTSKNQFSLKLSSVTAADTAVYYCATGYYYGSGFWGQGTLVTVSSGGSEGKSSGSGSESKSTGGSEIVLTQSPATLSLSPGERATLSCRASESVEYFGTSLMHWYQQKPGQPPRLLIYAASNVESGIPARFSGSGSGTDFTLTISSVEPEDFAVYFCQQTRKVPYTFGGGTKVEIK

SEQ ID NO: 321 - protein, 248 aa, synthetic construct
  REGION 1..248: note = Description of Artificial Sequence: Synthetic polypeptide
EIVLTQSPATLSLSPGERATLSCRASESVEYFGTSLMHWYQQKPGQPPRLLIYAASNVESGIPARFSGSGSGTDFTLTISSVEPEDFAVYFCQQTRKVPYTFGGGTKVEIKGGSEGKSSGSGSESKSTGGSQVQLQESGPGLVKPSQTLSLTCTVSGNSITSDYAWNWIRQPPGKGLEWIGYISYSGSTTYNPSLKSRVTISRDTSKNQFSLKLSSVTAADTAVYYCATGYYYGSGFWGQGTLVTVSS

SEQ ID NO: 322 - protein, 248 aa, synthetic construct
  REGION 1..248: note = Description of Artificial Sequence: Synthetic polypeptide
QVQLQESGPGLVKPSQTLSLTCTVSGNSITSDYAWNWIRQFPGKGLEWIGYISYSGSTTYNPSLKSRVTISRDTSKNQFSLKLSSVTAADTAVYYCATGYYYGSGFWGQGTLVTVSSGGSEGKSSGSGSESKSTGGSEIVLTQSPATLSLSPGERATLSCRASESVEYFGTSLMHWYQQKPGQPPRLLIYAASNVESGIPARFSGSGSGTDFTLTISSVEPEDFAVYFCQQTRKVPYTFGGGTKVEIK

SEQ ID NO: 323 - protein, 248 aa, synthetic construct
  REGION 1..248: note = Description of Artificial Sequence: Synthetic polypeptide
EIVLTQSPATLSLSPGERATLSCRASESVEYFGTSLMHWYQQKPGQPPRLLIYAASNVESGIPARFSGSGSGTDFTLTISSVEPEDFAVYFCQQTRKVPYTFGGGTKVEIKGGSEGKSSGSGSESKSTGGSQVQLQESGPGLVKPSQTLSLTCTVSGNSITSDYAWNWIRQFPGKGLEWIGYISYSGSTTYNPSLKSRVTISRDTSKNQFSLKLSSVTAADTAVYYCATGYYYGSGFWGQGTLVTVSS

SEQ ID NO: 324 - protein, 249 aa, synthetic construct
  REGION 1..249: note = Description of Artificial Sequence: Synthetic polypeptide
QVQLVESGGGVVQPGRSLRLSCAASGFTFSYYGMHWVRQAPGKGLEWVAFISYDGSNKYYADSVKGRFTISRDNSKNTLYLQMNSLRAEDTAVYYCAHLPYSGSYWAFDYWGQGTQVTVSSGGSEGKSSGSGSESKSTGGSQSVLTQPPSVSVAPGQTASITCGGDNIGSKSVHWYQQKPGQAPVLVVYDNSDRPSGIPERFSGSNSGTTATLTISRVEAGDEADYYCQVWDSSSDHPVVFGGGTKVTV

SEQ ID NO: 325 - protein, 249 aa, synthetic construct
  REGION 1..249: note = Description of Artificial Sequence: Synthetic polypeptide
QSVLTQPPSVSVAPGQTASITCGGDNIGSKSVHWYQQKPGQAPVLVVYDNSDRPSGIPERFSGSNSGTTATLTISRVEAGDEADYYCQVWDSSSDHPVVFGGGTKVTVGGSEGKSSGSGSESKSTGGSQVQLVESGGGVVQPGRSLRLSCAASGFTFSYYGMHWVRQAPGKGLEWVAFISYDGSNKYYADSVKGRFTISRDNSKNTLYLQMNSLRAEDTAVYYCAHLPYSGSYWAFDYWGQGTQVTVSS

SEQ ID NO: 326 - protein, 475 aa, synthetic construct
  REGION 1..475: note = Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSGGSEGKSSGSGSESKSTGGSDIQMTQSPSSLSASVGDRVTITCRARQSIGTAIHWYQQKPGKAPKLLIYYASESISGVPSRFSGSGSGTDFTLTISSVQPEDFATYYCQQSGSWPYTFGQGTKLEIKEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 327 - protein, 475 aa, synthetic construct
  REGION 1..475: note = Description of Artificial Sequence: Synthetic polypeptide
DIQMTQSPSSLSASVGDRVTITCRARQSIGTAIHWYQQKPGKAPKLLIYYASESISGVPSRFSGSGSGTDFTLTISSVQPEDFATYYCQQSGSWPYTFGQGTKLEIKGGSEGKSSGSGSESKSTGGSEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 328 - protein, 243 aa, synthetic construct
  REGION 1..243: note = Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSGGSEGKSSGSGSESKSTGGSDIQMTQSPSSLSASVGDRVTITCRARQSIGTAIHWYQQKPGKAPKLLIYYASESISGVPSRFSGSGSGTDFTLTISSVQPEDFATYYCQQSGSWPYTFGQGTKLEIK

SEQ ID NO: 329 - protein, 243 aa, synthetic construct
  REGION 1..243: note = Description of Artificial Sequence: Synthetic polypeptide
DIQMTQSPSSLSASVGDRVTITCRARQSIGTAIHWYQQKPGKAPKLLIYYASESISGVPSRFSGSGSGTDFTLTISSVQPEDFATYYCQQSGSWPYTFGQGTKLEIKGGSEGKSSGSGSESKSTGGSEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSS

SEQ ID NO: 330 - protein, 243 aa, synthetic construct
  REGION 1..243: note = Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSGGSEGKSSGSGSESKSTGGSDIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIK

SEQ ID NO: 331 - protein, 243 aa, synthetic construct
  REGION 1..243: note = Description of Artificial Sequence: Synthetic polypeptide
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIKGGSEGKSSGSGSESKSTGGSEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSS

SEQ ID NO: 332 - protein, 243 aa, synthetic construct
  REGION 1..243: note = Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSGGSEGKSSGSGSESKSTGGSDIQMTQSPSSLSASVGDRVTITCRARQSIGTAIHWYQQKPGKAPKLLIKYASESISGVPSRFSGSGSGTDFTLTISSVQPEDFATYYCQQSGSWPYTFGQGTKLEIK

SEQ ID NO: 333 - protein, 243 aa, synthetic construct
  REGION 1..243: note = Description of Artificial Sequence: Synthetic polypeptide
DIQMTQSPSSLSASVGDRVTITCRARQSIGTAIHWYQQKPGKAPKLLIKYASESISGVPSRFSGSGSGTDFTLTISSVQPEDFATYYCQQSGSWPYTFGQGTKLEIKGGSEGKSSGSGSESKSTGGSEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSS

SEQ ID NO: 334 - protein, 243 aa, synthetic construct
  REGION 1..243: note = Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSGGSEGKSSGSGSESKSTGGSDIQMTQSPSSLSASVGDRVTITCRARQSIGTAIHWYQQKPGKAPKLLIYYASESISGVPSRFSGSGSGTDFTLTISSLQPEDFATYYCQQSGSWPYTFGQGTKLEIK

SEQ ID NO: 335 - protein, 243 aa, synthetic construct
  REGION 1..243: note = Description of Artificial Sequence: Synthetic polypeptide
DIQMTQSPSSLSASVGDRVTITCRARQSIGTAIHWYQQKPGKAPKLLIYYASESISGVPSRFSGSGSGTDFTLTISSLQPEDFATYYCQQSGSWPYTFGQGTKLEIKGGSEGKSSGSGSESKSTGGSEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSS

SEQ ID NO: 336 - protein, 243 aa, synthetic construct
  REGION 1..243: note = Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSGGSEGKSSGSGSESKSTGGSDILLTQSPGILSVSPGERVSFSCRARQSIGTAIHWYQQRTNGSPRLLIKYASESISGIPSRFSGSGSGTDFTLTINSVESEDIADYYCQQSGSWPYTFGGGTKLEIK

SEQ ID NO: 337 - protein, 243 aa, synthetic construct
  REGION 1..243: note = Description of Artificial Sequence: Synthetic polypeptide
DILLTQSPGILSVSPGERVSFSCRARQSIGTAIHWYQQRTNGSPRLLIKYASESISGIPSRFSGSGSGTDFTLTINSVESEDIADYYCQQSGSWPYTFGGGTKLEIKGGSEGKSSGSGSESKSTGGSEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSS

SEQ ID NO: 338 - protein, 432 aa, synthetic construct
  REGION 1..432: note = Description of Artificial Sequence: Synthetic polypeptide
FKIPIEELEDRVFVNCNTSITWVEGTVGTLLSDITRLDLGKRILDPRGIYRCNGTDIYKDKESTVQVHYRMGSADDAKKDAAKKDDAKKDDAKKDGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGLNDIFEAQKIEWHE

SEQ ID NO: 339 - protein, 426 aa, synthetic construct
  REGION 1..426: note = Description of Artificial Sequence: Synthetic polypeptide
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVGSADDAKKDAAKKDDAKKDDAKKDGSQSIKGNHLVKVYDYQEDGSVLLTCDAEAKNITWFKDGKMIGFLTEDKKKWNLGSNAKDPRGMYQCKGSQNKSKPLQVYYRMGSGSLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTFPPSQEEMTKNQVSLRCLVKGFYPSDIAVEWESNGQPENNYKTTKPVLDSDGSFRLESRLTVDKSRWQEGNVFSCSVMHEALHNHYTQKSLSLSGGHHHHHH

SEQ ID NO: 340 - protein, 805 aa, synthetic construct
  REGION 1..805: note = Description of Artificial Sequence: Synthetic polypeptide
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVGSADDAKKDAAKKDDAKKDDAKKDGSQSIKGNHLVKVYDYQEDGSVLLTCDAEAKNITWFKDGKMIGFLTEDKKKWNLGSNAKDPRGMYQCKGSQNKSKPLQVYYRMGGGSDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSHHHHHHHH

SEQ ID NO: 341 - protein, 9 aa, Homo sapiens
PQYPGSEIL

SEQ ID NO: 342 - protein, 262 aa, synthetic construct
  REGION 1..262: note = Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSGTEGKSSGSGSESKSTDILLTQSPGILSVSPGERVSFSCRARQSIGTAIHWYQQRTNGSPRLLIKYASESISGIPSRFSGSGSGTDFTLTINSVESEDIADYYCQQSNSWPYTFGGGTKLEIKGPGGQHHHHHHGAYPYDVPDYAS

SEQ ID NO: 343 - protein, 262 aa, synthetic construct
  REGION 1..262: note = Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSGTEGKSSGSGSESKSTDIQMTQSPSSLSASVGDRVTITCRARQSIGTAIHWYQQKPGKAPKLLIYYASESISGVPSRFSGSGSGTDFTLTISSLQPEDFATYYCQQSNSWPYTFGQGTKLEIKGPGGQHHHHHHGAYPYDVPDYAS

SEQ ID NO: 344 - protein, 262 aa, synthetic construct
  REGION 1..262: note = Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSGTEGKSSGSGSESKSTDIQMTQSPSSLSASVGDRVTITCRARQSIGTAIHWYQQKPGKAPKLLIKYASESISGVPSRFSGSGSGTDFTLTISSLQPEDFATYYCQQSNSWPYTFGQGTKLEIKGPGGQHHHHHHGAYPYDVPDYAS

SEQ ID NO: 345 - protein, 262 aa, synthetic construct
  REGION 1..262: note = Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSGTEGKSSGSGSESKSTDIQMTQSPSSLSASVGDRVTITCRARQSIGTAIHWYQQKPGKAPKLLIKYASESISGVPSRFSGSGSGTDFTLTISSVQPEDFATYYCQQSNSWPYTFGQGTKLEIKGPGGQHHHHHHGAYPYDVPDYAS

SEQ ID NO: 346 - protein, 262 aa, synthetic construct
  REGION 1..262: note = Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSGTEGKSSGSGSESKSTDIQMTQSPSSLSASVGDRVTITCRARQSIGTAIHWYQQKPGKAPKLLIYYASESISGVPSRFSGSGSGTDFTLTISSVQPEDFATYYCQQSGSWPYTFGQGTKLEIKGPGGQHHHHHHGAYPYDVPDYAS

SEQ ID NO: 347 - protein, 262 aa, synthetic construct
  REGION 1..262: note = Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSGTEGKSSGSGSESKSTDIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIKGPGGQHHHHHHGAYPYDVPDYAS

SEQ ID NO: 348 - protein, 262 aa, synthetic construct
  REGION 1..262: note = Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSGTEGKSSGSGSESKSTDIQMTQSPSSLSASVGDRVTITCRARQSIGTAIHWYQQKPGKAPKLLIKYASESISGVPSRFSGSGSGTDFTLTISSVQPEDFATYYCQQSGSWPYTFGQGTKLEIKGPGGQHHHHHHGAYPYDVPDYAS

SEQ ID NO: 349 - protein, 262 aa, synthetic construct
  REGION 1..262: note = Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSGTEGKSSGSGSESKSTDIQMTQSPSSLSASVGDRVTITCRARQSIGTAIHWYQQKPGKAPKLLIYYASESISGVPSRFSGSGSGTDFTLTISSLQPEDFATYYCQQSGSWPYTFGQGTKLEIKGPGGQHHHHHHGAYPYDVPDYAS

SEQ ID NO: 350 - protein, 262 aa, synthetic construct
  REGION 1..262: note = Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSGTEGKSSGSGSESKSTDILLTQSPGILSVSPGERVSFSCRARQSIGTAIHWYQQRTNGSPRLLIKYASESISGIPSRFSGSGSGTDFTLTINSVESEDIADYYCQQSGSWPYTFGGGTKLEIKGPGGQHHHHHHGAYPYDVPDYAS

SEQ ID NO: 351 - protein, 479 aa, synthetic construct
  REGION 1..479: note = Description of Artificial Sequence: Synthetic polypeptide
EIVLTQSPATLSLSPGERATLSCRASESVEYFGTSLMHWYQQKPGQPPRLLIYAASNVESGIPARFSGSGSGTDFTLTISSVEPEDFAVYFCQQTRKVPYTFGGGTKVEIKGGSEGKSSGSGSESKSTGGSQVQLQESGPGLVKPSQTLSLTCTVSGNSITSDYAWNWIRQFPGKRLEWIGYISYSGSTTYNPSLKSRVTISRDTSKNQFSLKLSSVTAADTAVYYCATGYYYGSGFWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 352 - protein, 482 aa, synthetic construct
  REGION 1..482: note = Description of Artificial Sequence: Synthetic polypeptide
EIVLTQSPSFLSASVGDRVTITCRASQGISSYLSWYQQKPGKAPKLLIYATSTLQSGVPSRFSGSGSGTEFTLTISSLQPEDFATYYCQQLNSYPRTFGQGTKVEIKGGSEGKSSGSGSESKSTGGSEVQLVESGGGLVQPGGSLRLSCAASGFTFSSYWMTWVRQAPGKGLEWVANIKQDGSERYYVDSVKGRFTISRDNAKNSLYLQMNSLRAEDTAVYYCARDQNYDILTGHYGMDVWGQGTTVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 353 - protein, 481 aa, synthetic construct
  REGION 1..481: note = Description of Artificial Sequence: Synthetic polypeptide
QSVLTQPPSVSVAPGQTASITCGGDNIGSKSVHWYQQKPGQAPVLVVYDNSDRPSGIPERFSGSNSGTTATLTISRVEAGDEADYYCQVWDSSSDHPVVFGGGTKVTVLGGSEGKSSGSGSESKSTGGSQVQLVESGGGVVQPGRSLRLSCAASGFTFSYYGMHWVRQAPGKGLEWVAFISYDGSNKYYADSVKGRFTISRDNSKNTLYLQMNSLRAEDTAVYYCAHLPYSGSYWAFDYWGQGTQVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 354 - protein, 454 aa, synthetic construct
  REGION 1..454: note = Description of Artificial Sequence: Synthetic polypeptide
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 355 - protein, 448 aa, synthetic construct
  REGION 1..448: note = Description of Artificial Sequence: Synthetic polypeptide
QVQLQESGPGLVKPSETLSLTCTVSGGSISSYYWSWLRQPAGSGLEWIGRLYVSGFTNYNPSLKSRVTLSLDPSRNQLSLKLSSVTAADTAVYYCAGDSGNYWGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 356 - protein, 452 aa, synthetic construct
  REGION 1..452: note = Description of Artificial Sequence: Synthetic polypeptide
EVQLVESGGGVVQPGRSLRLSCVASGFTFSSYDIHWVRQAPGKGLEWVAIISYDGSKKDYTDSVKGRFTISRDNSKNTLYLQMDSLRVEDSAVYSCARESGWSHYYYYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 357 - protein, 484 aa, synthetic construct
  REGION 1..484: note = Description of Artificial Sequence: Synthetic polypeptide
SSELTQPPSVSVAPGQTARITCGGNNIGSKSVHWYQQKPGQAPVLVVYDDSDRPSGIPERFSGSNSGNTATLTISRVEAGDEADYYCQVWDSSSDHVVFGGGTKLTVLGGSEGKSSGSGSESKSTGGSQVQLVESGGGLVQPGGSLRLSCAASGFTFSHYAMSWVRQAPGKGLEWVSTIGGSGGSTYYADSVKGRFTISRDNSKNTLYLQMNSLRAEDTAVYYCAKPHIVMVTALLYDGMDVWGQGTMVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 358 - protein, 445 aa, synthetic construct
  REGION 1..445: note = Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 359 - protein, 449 aa, synthetic construct
  REGION 1..449: note = Description of Artificial Sequence: Synthetic polypeptide
QVQLQQSGPRLVRPSQTLSLTCAISGDSVFNNNAAWSWIRQSPSRGLEWLGRTYYRSKWLYDYAVSVKSRITVNPDTSRNQFTLQLNSVTPEDTALYYCARGYSSSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 360 - protein, 474 aa, synthetic construct
  REGION 1..474: note = Description of Artificial Sequence: Synthetic polypeptide
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIKGGSEGKSSGSGSESKSTGGSEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 361 - protein, 455 aa, synthetic construct
  REGION 1..455: note = Description of Artificial Sequence: Synthetic polypeptide
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 362 - protein, 475 aa, synthetic construct
  REGION 1..475: note = Description of Artificial Sequence: Synthetic polypeptide
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIKGGSEGKSSGSGSESKSTGGSEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 363 - DNA, 1437 bp, synthetic construct
  misc_feature 1..1437: note = Description of Artificial Sequence: Synthetic polynucleotide
  source 1..1437: mol_type = other DNA
gagattgttctcacccaatccccagctactctctctctttcacccggtgagcgggcaaccctctcctgtagagccagcgagagcgtggagtattttggcacatccctgatgcactggtatcagcaaaaaccaggacaaccccccagactcctcatatatgccgcctcaaatgtcgagagtgggatacctgcacggttttcaggaagcggcagcggtactgacttcacattgactatatcctctgtagagccagaggattttgcagtctacttctgccagcaaactaggaaggttccatatacttttgggggcggtacaaaagttgagataaagggcggctccgagggcaagagcagcggcagcggcagcgagagcaagagcaccggcggcagccaagtacagctccaggagtcaggacctgggctcgtcaaaccatctcagacattgtccctgacatgcacagtttccggcaacagtattacttccgactatgcttggaattggatcaggcaattcccaggaaagcggctcgagtggataggttatatttcttactctggatctactacctacaatcccagtttgaagtctcgcgtgacaattagccgggacacatcaaaaaatcaattctcacttaaacttagttctgtaaccgctgccgatacagccgtgtactactgcgccactggttattattatggaagcggattttgggggcaaggaactttggtgaccgtctcttccgagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacgtgctgcccccatcccgggaggagatgaccaagaaccaggtcagcctgctgtgcctggtcaaaggcttctatcccagcgacatcgccgtggagtgggagagcaatgggcagccggagaacaactacctcacctggcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggt

SEQ ID NO: 364 - DNA, 1356 bp, synthetic construct
  misc_feature 1..1356: note = Description of Artificial Sequence: Synthetic polynucleotide
  source 1..1356: mol_type = other DNA
gaggtacaacttgtcgaaagtggcggtggagtcgtccagcctgggcgatcacttcgcctctcctgtgtagcctctggtttcactttctcatcttacgacatacactgggtccgccaggcacctggtaaggggctggagtgggttgccatcattagttacgatggctccaaaaaagattacaccgatagcgtaaagggcagatttaccatttccagggataattcaaagaacaccctgtatctgcaaatggacagcctccgcgtcgaagactctgcagtttatagctgtgccagggagtcaggctggtcccattattactattatggtatggacgtttggggccagggaaccatggtcactgttagttcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacgtgctgcccccatcccgggaggagatgaccaagaaccaggtcagcctgctgtgcctggtcaaaggcttctatcccagcgacatcgccgtggagtgggagagcaatgggcagccggagaacaactacctcacctggcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggt

SEQ ID NO: 365 - DNA, 1443 bp, synthetic construct
  misc_feature 1..1443: note = Description of Artificial Sequence: Synthetic polynucleotide
  source 1..1443: mol_type = other DNA
cagagcgtacttacccagcctcccagcgtgtctgtagccccaggacagacagccagtattacatgcggtggtgacaatataggttccaaatccgtgcattggtaccagcagaagccagggcaagctcccgtgctcgtggtatatgataattccgaccgcccttccggcattcccgaacggtttagtggttcaaattcaggcaccacagcaactctgaccataagcagagtcgaagctggagacgaagccgactactactgtcaggtatgggactctagtagtgaccaccctgtcgtcttcggtgggggaaccaaagtgaccgttctgggcggctccgagggcaagagcagcggcagcggcagcgagagcaagagcaccggcggcagccaggtccagctcgtagaaagtgggggcggcgtagttcagccaggcaggagtctccggctgagttgtgcagccagcggctttactttttcctactatggaatgcactgggtacgtcaggcacccggcaaaggtttggagtgggtcgcattcatttcttatgatggatcaaataagtattatgccgatagtgtaaagggcagatttacaataagtcgagacaactcaaagaacactctctacctccaaatgaatagtcttcgggcagaggatactgcagtgtactattgtgctcatcttccttattccggttcttactgggcattcgattattgggggcaagggacacaagttaccgtgtctagcgagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacgtgctgcccccatcccgggaggagatgaccaagaaccaggtcagcctgctgtgcctggtcaaaggcttctatcccagcgacatcgccgtggagtgggagagcaatgggcagccggagaacaactacctcacctggcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggt

SEQ ID NO: 366 - DNA, 1452 bp, synthetic construct
  misc_feature 1..1452: note = Description of Artificial Sequence: Synthetic polynucleotide
  source 1..1452: mol_type = other DNA
agcagcgaattgacccaaccaccttccgtcagcgtcgcaccagggcaaaccgcccgcatcacatgcggtgggaacaatataggaagcaaatctgtccactggtaccagcaaaaaccaggacaagcccctgttctggtcgtctatgatgacagcgacagaccaagtggtattcccgagagattctccggtagcaactctggaaatacagctactttgaccatctccagagttgaggctggtgacgaggcagattactattgccaggtctgggacagctccagcgaccacgtcgtattcggtggcgggaccaagctgactgtgctgggcggctccgagggcaagagcagcggcagcggcagcgagagcaagagcaccggcggcagccaggtgcagttggtagagtcaggagggggcctcgttcaacctggtggcagcctccgtttgtcttgtgctgccagtggatttactttcagtcactacgcaatgagctgggtgagacaagcacctggcaagggccttgagtgggtctccactatcggcggttcaggggggagcacttactacgctgactctgtaaaaggtcgctttactatatctagagataactctaaaaacacactctacttgcagatgaacagcctgcgagccgaagatacagccgtgtactactgcgccaagcctcatattgtaatggtcactgccctcttgtatgatggcatggatgtttggggccaagggacaatggtgacagtctcaagcgagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacgtgctgcccccatcccgggaggagatgaccaagaaccaggtcagcctgctgtgcctggtcaaaggcttctatcccagcgacatcgccgtggagtgggagagcaatgggcagccggagaacaactacctcacctggcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggt

SEQ ID NO: 367 - DNA, 1362 bp, synthetic construct
  misc_feature 1..1362: note = Description of Artificial Sequence: Synthetic polynucleotide
  source 1..1362: mol_type = other DNA
caggttcaacttcaagaatccgggccaggtctggtcaagccttcagagactttgtcccttacttgcacagtgagcggtggctctatctcaagttactactggtcatggatacggcagcccccaggaaaggggcttgagtggattgggtacatttattactcagggtcaacaaactacaatccctccctcaaatcccgagtgacaattagtgtcgatacatctaaaaaccagttttccctgaaattgagctcagtcaccgcagctgatactgcagtctattattgtgctggcacaacaatcttcggggtagtaactccaaacttctactacgggatggacgtgtgggggcaaggaacaaccgtaacagtaagtagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacgtgctgcccccatcccgggaggagatgaccaagaaccaggtcagcctgctgtgcctggtcaaaggcttctatcccagcgacatcgccgtggagtgggagagcaatgggcagccggagaacaactacctcacctggcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggt

SEQ ID NO: 368 - DNA, 1356 bp, synthetic construct; identical to SEQ ID NO: 364
  misc_feature 1..1356: note = Description of Artificial Sequence: Synthetic polynucleotide
  source 1..1356: mol_type = other DNA

SEQ ID NO: 369 - DNA, 1344 bp, synthetic construct
  misc_feature 1..1344: note = Description of Artificial Sequence: Synthetic polynucleotide
  source 1..1344: mol_type = other DNA
caagtacaacttcaagagtctggccctgggcttgttaagccctcagagaccttgtcactgacctgtaccgtatcaggcgggtcaatttcatcttactactggagttggcttcgtcagcctgccggatctggactggagtggataggtagactgtatgtttccggctttacaaattacaacccatctttgaaaagccgtgtgactctcagcctcgacccttctcggaatcaactttcacttaaattgtcttctgttacagctgccgacactgcagtatattattgtgcaggggactcaggcaactattggggatggtttgatccttgggggcaggggaccctggtaaccgtgagttctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacgtgctgcccccatcccgggaggagatgaccaagaaccaggtcagcctgctgtgcctggtcaaaggcttctatcccagcgacatcgccgtggagtgggagagcaatgggcagccggagaacaactacctcacctggcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggt

SEQ ID NO: 370 - DNA, 1365 bp, synthetic construct
  misc_feature 1..1365: note = Description of Artificial Sequence: Synthetic polynucleotide
  source 1..1365: mol_type = other DNA
caggttcagctgcaagagtctggccctggcctggtcaagccttccgagacactgtctctgacctgcaccgtgtctggcggctccatctcctcctactactggtcctggatcagacagcctcctggcaaaggcctggaatggatcggctacatctactactccggctccaccaactacaaccccagcctgaagtccagagtgaccatctccgtggacacctccaagaaccagttctccctgaagctgtcctccgtgaccgctgctgataccgccgtgtactattgtgctggcaccaccatcttcggcgtggtcacccctaacttctactacggcatggacgtgtggggccaaggcacaacagtgacagtctcttctgcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacttgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacgtgctgcccccatcccgggaggagatgaccaagaaccaggtcagcctgctgtgcctggtcaaaggcttctatcccagcgacatcgccgtggagtgggagagcaatgggcagccggagaacaactacctcacctggcctcccgtgctggactccgacggctccttcttcctctacagcaagctcaccgtggacaagtccagatggcagcaggggaacgtcttctcatgctccgtgatgcatgaggctctgcacaaccactacacgcagaagtctctctccctgtctccgggaaaa

SEQ ID NO: 371 - DNA, 1335 bp, synthetic construct
  misc_feature 1..1335: note = Description of Artificial Sequence: Synthetic polynucleotide
  source 1..1335: mol_type = other DNA
gaggtgcaactggtggagtctgggggaggcctggtcaagcctggggggtccctgagactctcctgtgcagcctctggattcaccttcagtagatataacatgaactgggtccgccaggctccagggaaggggctggagtgggtctcatccattagtactagtagtaattacatatactacgcagactcagtgaagggccgattcaccttctccagagacaacgccaagaactcactggatctgcaaatgagcggcctgagagccgaggacacggctatttattactgtacgagaggctgggggccttttgactactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacgtgtaccccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcgccctcgtgagcaagctcaccgtggacaagtctagatggcagcaggggaacgtcttctcatgctccgtgatgcatgaggctctgcacaaccactacacgcagaagagcctctccctgtctccgggt

SEQ ID NO: 372 - DNA, 1347 bp, synthetic construct
  misc_feature 1..1347: note = Description of Artificial Sequence: Synthetic polynucleotide
  source 1..1347: mol_type = other DNA
caggtgcagctccaacagagtggtcccagactcgtgagaccctctcaaacactcagtttgacttgtgccatctcaggcgattcagttttcaacaacaatgcagcttggagctggattaggcagtcacctagtcgcggtcttgaatggcttgggcgtacatactatcgctctaaatggttgtatgattacgctgtgtccgtgaagagccgaatcaccgtaaaccctgatacctccaggaatcagttcacattgcaactgaatagtgtgactcccgaggatactgcactctattattgtgcccgaggatatagcagtagcttcgactattggggacaagggacactcgttaccgttagttcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacgtgtaccccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcgccctcgtgagcaagctcaccgtggacaagtctagatggcagcaggggaacgtcttctcatgctccgtgatgcatgaggctctgcacaaccactacacgcagaagagcctctccctgtctccgggt

SEQ ID NO: 373 - DNA, 1422 bp, synthetic construct
  misc_feature 1..1422: note = Description of Artificial Sequence: Synthetic polynucleotide
  source 1..1422: mol_type = other DNA
gacatacaaatgacacaatcaccctcttctctttctgcaagcgttggcgaccgtgtcactatcacttgtcgagcccgccagtccataggtactgccattcactggtatcaacagaagcctggcaaggctcccaaactcctgattaagtatgccagcgagagcatttccggcgtaccttcaagattttccggctccggtagtgggacagatttcactctcactatatctagcctccaaccagaagatttcgccacttactactgtcaacaatcaggttcatggccttacactttcggccaggggacaaaattggagatcaagggcggctccgagggcaagagcagcggcagcggcagcgagagcaagagcaccggcggcagcgaggtgcaactggtggagtctgggggaggcctggtcaagcctggggggtccctgagactctcctgtgcagcctctggattcaccttcagtagatataacatgaactgggtccgccaggctccagggaaggggctggagtgggtctcatccattagtactagtagtaattacatatactacgcagactcagtgaagggccgattcaccttctccagagacaacgccaagaactcactggatctgcaaatgagcggcctgagagccgaggacacggctatttattactgtacgagaggctgggggccttttgactactggggccagggaaccctggtcaccgtctcctcagagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacgtgtaccccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcgccctcgtgagcaagctcaccgtggacaagtctagatggcagcaggggaacgtcttctcatgctccgtgatgcatgaggctctgcacaaccactacacgcagaagagcctctccctgtctccgggt

SEQ ID NO: 374 - DNA, 1425 bp, synthetic construct
  misc_feature 1..1425: note = Description of Artificial Sequence: Synthetic polynucleotide
  source 1..1425: mol_type = other DNA
gacatccagatgacccagtctccatcctctctgtccgcctctgtgggcgacagagtgaccattacctgccgggccagacagtctatcggcaccgctatccactggtatcagcagaagcctggcaaggcccctaagctgctgattaagtacgcctccgagtccatctccggcgtgccctccagattttctggctctggatctggcaccgactttaccctgacaatctccagcctgcagcctgaggacttcgccacctactactgtcagcagtccggctcttggccttacacctttggtcagggcaccaagctggaaatcaagggcggatctgagggaaagtccagcggctccggcagcgaaagcaagtccaccggcggaagcgaggtgcagctggttgaatctggcggaggactggttaagcctggcggctctctgagactgtcttgtgctgcttctggcttcaccttcagccggtacaacatgaactgggtccgacaggctcctggcaaaggcctggaatgggtgtcctccatctccacctccagcaactacatctactacgccgactccgtgaagggcagattcaccttctccagagacaacgccaagaactccctggacctgcagatgtctggcctgagagctgaggacaccgctatctactactgcaccagaggctggggacccttcgattattggggccagggaaccctggtcaccgtgtcatctgagcccaaatctagcgacaaaactcacacttgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacgtgtaccccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcgccctcgtgagcaagctcaccgtggacaagtccagatggcagcaggggaacgtcttctcatgctccgtgatgcatgaggctctgcacaaccactacacgcagaagtctctctccctgtctccgggaaaa

SEQ ID NO: 375 - protein, 475 aa, synthetic construct
  REGION 1..475: note = Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSGGSEGKSSGSGSESKSTGGSDIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIKEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 376 - protein, 475 aa, synthetic construct; identical to SEQ ID NO: 362
  REGION 1..475: note = Description of Artificial Sequence: Synthetic polypeptide

SEQ ID NO: 377 - protein, 475 aa, synthetic construct
  REGION 1..475: note = Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSGGSEGKSSGSGSESKSTGGSDIQMTQSPSSLSASVGDRVTITCRARQSIGTAIHWYQQKPGKAPKLLIKYASESISGVPSRFSGSGSGTDFTLTISSVQPEDFATYYCQQSGSWPYTFGQGTKLEIKEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 378 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 379 - protein, 475 aa, synthetic construct
  REGION 1..475: note = Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSGGSEGKSSGSGSESKSTGGSDIQMTQSPSSLSASVGDRVTITCRARQSIGTAIHWYQQKPGKAPKLLIYYASESISGVPSRFSGSGSGTDFTLTISSLQPEDFATYYCQQSGSWPYTFGQGTKLEIKEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 380 - protein, 475 aa, synthetic construct
  REGION 1..475: note = Description of Artificial Sequence: Synthetic polypeptide
DIQMTQSPSSLSASVGDRVTITCRARQSIGTAIHWYQQKPGKAPKLLIYYASESISGVPSRFSGSGSGTDFTLTISSLQPEDFATYYCQQSGSWPYTFGQGTKLEIKGGSEGKSSGSGSESKSTGGSEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 381 - protein, 475 aa, synthetic construct
  REGION 1..475: note = Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSGGSEGKSSGSGSESKSTGGSDILLTQSPGILSVSPGERVSFSCRARQSIGTAIHWYQQRTNGSPRLLIKYASESISGIPSRFSGSGSGTDFTLTINSVESEDIADYYCQQSGSWPYTFGGGTKLEIKEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 382 - protein, 475 aa, synthetic construct
  REGION 1..475: note = Description of Artificial Sequence: Synthetic polypeptide
DILLTQSPGILSVSPGERVSFSCRARQSIGTAIHWYQQRTNGSPRLLIKYASESISGIPSRFSGSGSGTDFTLTINSVESEDIADYYCQQSGSWPYTFGGGTKLEIKGGSEGKSSGSGSESKSTGGSEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 383 - skipped

SEQ ID NO: 384 - DNA, 729 bp, synthetic construct
  misc_feature 1..729: note = Description of Artificial Sequence: Synthetic polynucleotide
  source 1..729: mol_type = other DNA
gaggtgcagctggtggagagcggtggcggtctggtgaagccaggtggcagcctgcgcctgagctgtgccgccagcggtttcaccttcagccgctacaacatgaactgggtgcgccaagccccaggcaagggcctggagtgggtgagcagcatcagcaccagcagcaactacatctactacgccgacagcgtgaagggccgcttcaccttcagccgcgacaacgccaagaacagcctggacctgcagatgagcggtctgcgcgccgaggacaccgccatctactactgcacccgcggttggggcccattcgactactggggccagggcaccctggtgaccgtgagcagcggcggatctgagggaaagtccagcggctccggcagcgaaagcaagtccaccggcggaagcgacatccagatgacccagagcccaagcagcctgagcgccagcgtcggcgaccgcgtgaccatcacctgtcgtgcccgccagagcatcggcaccgccatccactggtaccagcagaagccaggcaaggccccaaagctgctgatctactacgccagcgagagcatcagcggtgtgccaagccgcttcagcggcagcggcagcggcaccgacttcaccctgaccatcagcagcgtgcagccagaggacttcgccacctactactgccagcagagcggcagctggccatacaccttcggccagggcaccaagctggagatcaag

SEQ ID NO: 385 - DNA, 729 bp, synthetic construct
  misc_feature 1..729: note = Description of Artificial Sequence: Synthetic polynucleotide
  source 1..729: mol_type = other DNA
gacatccagatgacccagagcccaagcagcctgagcgccagcgtcggcgaccgcgtgaccatcacctgtcgtgcccgccagagcatcggcaccgccatccactggtaccagcagaagccaggcaaggccccaaagctgctgatctactacgccagcgagagcatcagcggtgtgccaagccgcttcagcggcagcggcagcggcaccgacttcaccctgaccatcagcagcgtgcagccagaggacttcgccacctactactgccagcagagcggcagctggccatacaccttcggccagggcaccaagctggagatcaagggcggatctgagggaaagtccagcggctccggcagcgaaagcaagtccaccggcggaagcgaggtgcagctggtggagagcggtggcggtctggtgaagccaggtggcagcctgcgcctgagctgtgccgccagcggtttcaccttcagccgctacaacatgaactgggtgcgccaagccccaggcaagggcctggagtgggtgagcagcatcagcaccagcagcaactacatctactacgccgacagcgtgaagggccgcttcaccttcagccgcgacaacgccaagaacagcctggacctgcagatgagcggtctgcgcgccgaggacaccgccatctactactgcacccgcggttggggcccattcgactactggggccagggcaccctggtgaccgtgagcagc

SEQ ID NO: 386 - DNA, 729 bp, synthetic construct
  misc_feature 1..729: note = Description of Artificial Sequence: Synthetic polynucleotide
  source 1..729: mol_type = other DNA
gaggtgcagctggtggagagcggtggcggtctggtgaagccaggtggcagcctgcgcctgagctgtgccgccagcggtttcaccttcagccgctacaacatgaactgggtgcgccaagccccaggcaagggcctggagtgggtgagcagcatcagcaccagcagcaactacatctactacgccgacagcgtgaagggccgcttcaccttcagccgcgacaacgccaagaacagcctggacctgcagatgagcggtctgcgcgccgaggacaccgccatctactactgcacccgcggttggggcccattcgactactggggccagggcaccctggtgaccgtgagcagcggcggatctgagggaaagtccagcggctccggcagcgaaagcaagtccaccggcggaagcgacatccagatgacccagagcccaagcagcctgagcgccagcgtcggcgaccgcgtgaccatcacctgtcgtgcccgccagagcatcggcaccgccatccactggtaccagcagaagccaggcaaggccccaaagctgctgatcaagtacgccagcgagagcatcagcggtgtgccaagccgcttcagcggcagcggcagcggcaccgacttcaccctgaccatcagcagcctgcagccagaggacttcgccacctactactgccagcagagcggcagctggccatacaccttcggccagggcaccaagctggagatcaag

SEQ ID NO: 387 - DNA, 729 bp, synthetic construct
  misc_feature 1..729: note = Description of Artificial Sequence: Synthetic polynucleotide
  source 1..729: mol_type = other DNA
gacatccagatgacccagagcccaagcagcctgagcgccagcgtcggcgaccgcgtgaccatcacctgtcgtgcccgccagagcatcggcaccgccatccactggtaccagcagaagccaggcaaggccccaaagctgctgatcaagtacgccagcgagagcatcagcggtgtgccaagccgcttcagcggcagcggcagcggcaccgacttcaccctgaccatcagcagcctgcagccagaggacttcgccacctactactgccagcagagcggcagctggccatacaccttcggccagggcaccaagctggagatcaagggcggatctgagggaaagtccagcggctccggcagcgaaagcaagtccaccggcggaagcgaggtgcagctggtggagagcggtggcggtctggtgaagccaggtggcagcctgcgcctgagctgtgccgccagcggtttcaccttcagccgctacaacatgaactgggtgcgccaagccccaggcaagggcctggagtgggtgagcagcatcagcaccagcagcaactacatctactacgccgacagcgtgaagggccgcttcaccttcagccgcgacaacgccaagaacagcctggacctgcagatgagcggtctgcgcgccgaggacaccgccatctactactgcacccgcggttggggcccattcgactactggggccagggcaccctggtgaccgtgagcagc

SEQ ID NO: 388 - DNA, 729 bp, synthetic construct
  misc_feature 1..729: note = Description of Artificial Sequence: Synthetic polynucleotide
  source 1..729: mol_type = other DNA
gaggtgcagctggtggagagcggtggcggtctggtgaagccaggtggcagcctgcgcctgagctgtgccgccagcggtttcaccttcagccgctacaacatgaactgggtgcgccaagccccaggcaagggcctggagtgggtgagcagcatcagcaccagcagcaactacatctactacgccgacagcgtgaagggccgcttcaccttcagccgcgacaacgccaagaacagcctggacctgcagatgagcggtctgcgcgccgaggacaccgccatctactactgcacccgcggttggggcccattcgactactggggccagggcaccctggtgaccgtgagcagcggcggatctgagggaaagtccagcggctccggcagcgaaagcaagtccaccggcggaagcgacatccagatgacccagagcccaagcagcctgagcgccagcgtcggcgaccgcgtgaccatcacctgtcgtgcccgccagagcatcggcaccgccatccactggtaccagcagaagccaggcaaggccccaaagctgctgatcaagtacgccagcgagagcatcagcggtgtgccaagccgcttcagcggcagcggcagcggcaccgacttcaccctgaccatcagcagcgtgcagccagaggacttcgccacctactactgccagcagagcggcagctggccatacaccttcggccagggcaccaagctggagatcaag

SEQ ID NO: 389 - DNA, 729 bp, synthetic construct
  misc_feature 1..729: note = Description of Artificial Sequence: Synthetic polynucleotide
  source 1..729: mol_type = other DNA
gacatccagatgacccagagcccaagcagcctgagcgccagcgtcggcgaccgcgtgaccatcacctgtcgtgcccgccagagcatcggcaccgccatccactggtaccagcagaagccaggcaaggccccaaagctgctgatcaagtacgccagcgagagcatcagcggtgtgccaagccgcttcagcggcagcggcagcggcaccgacttcaccctgaccatcagcagcgtgcagccagaggacttcgccacctactactgccagcagagcggcagctggccatacaccttcggccagggcaccaagctggagatcaagggcggatctgagggaaagtccagcggctccggcagcgaaagcaagtccaccggcggaagcgaggtgcagctggtggagagcggtggcggtctggtgaagccaggtggcagcctgcgcctgagctgtgccgccagcggtttcaccttcagccgctacaacatgaactgggtgcgccaagccccaggcaagggcctggagtgggtgagcagcatcagcaccagcagcaactacatctactacgccgacagcgtgaagggccgcttcaccttcagccgcgacaacgccaagaacagcctggacctgcagatgagcggtctgcgcgccgaggacaccgccatctactactgcacccgcggttggggcccattcgactactggggccagggcaccctggtgaccgtgagcagc

SEQ ID NO: 390 - DNA, 729 bp, synthetic construct
  misc_feature 1..729: note = Description of Artificial Sequence: Synthetic polynucleotide
  source 1..729: mol_type = other DNA
gaggtgcagctggtggagagcggtggcggtctggtgaagccaggtggcagcctgcgcctgagctgtgccgccagcggtttcaccttcagccgctacaacatgaactgggtgcgccaagccccaggcaagggcctggagtgggtgagcagcatcagcaccagcagcaactacatctactacgccgacagcgtgaagggccgcttcaccttcagccgcgacaacgccaagaacagcctggacctgcagatgagcggtctgcgcgccgaggacaccgccatctactactgcacccgcggttggggcccattcgactactggggccagggcaccctggtgaccgtgagcagcggcggatctgagggaaagtccagcggctccggcagcgaaagcaagtccaccggcggaagcgacatccagatgacccagagcccaagcagcctgagcgccagcgtcggcgaccgcgtgaccatcacctgtcgtgcccgccagagcatcggcaccgccatccactggtaccagcagaagccaggcaaggccccaaagctgctgatctactacgccagcgagagcatcagcggtgtgccaagccgcttcagcggcagcggcagcggcaccgacttcaccctgaccatcagcagcctgcagccagaggacttcgccacctactactgccagcagagcggcagctggccatacaccttcggccagggcaccaagctggagatcaag

SEQ ID NO: 391 - DNA, 729 bp, synthetic construct
  misc_feature 1..729: note = Description of Artificial Sequence: Synthetic polynucleotide
  source 1..729: mol_type = other DNA
gacatccagatgacccagagcccaagcagcctgagcgccagcgtcggcgaccgcgtgaccatcacctgtcgtgcccgccagagcatcggcaccgccatccactggtaccagcagaagccaggcaaggccccaaagctgctgatctactacgccagcgagagcatcagcggtgtgccaagccgcttcagcggcagcggcagcggcaccgacttcaccctgaccatcagcagcctgcagccagaggacttcgccacctactactgccagcagagcggcagctggccatacaccttcggccagggcaccaagctggagatcaagggcggatctgagggaaagtccagcggctccggcagcgaaagcaagtccaccggcggaagcgaggtgcagctggtggagagcggtggcggtctggtgaagccaggtggcagcctgcgcctgagctgtgccgccagcggtttcaccttcagccgctacaacatgaactgggtgcgccaagccccaggcaagggcctggagtgggtgagcagcatcagcaccagcagcaactacatctactacgccgacagcgtgaagggccgcttcaccttcagccgcgacaacgccaagaacagcctggacctgcagatgagcggtctgcgcgccgaggacaccgccatctactactgcacccgcggttggggcccattcgactactggggccagggcaccctggtgaccgtgagcagc

SEQ ID NO: 392 - DNA, 729 bp, synthetic construct
  misc_feature 1..729: note = Description of Artificial Sequence: Synthetic polynucleotide
  source 1..729: mol_type = other DNA
gaggtgcaactggtggagtctgggggaggcctggtcaagcctggggggtccctgagactctcctgtgcagcctctggattcaccttcagtagatataacatgaactgggtccgccaggctccagggaaggggctggagtgggtctcatccattagtactagtagtaattacatatactacgcagactcagtgaagggccgattcaccttctccagagacaacgccaagaactcactggatctgcaaatgagcggcctgagagccgaggacacggctatttattactgtacgagaggctgggggccttttgactactggggccagggaaccctggtcaccgtctcctcaggcggatctgagggaaagtccagcggctccggcagcgaaagcaagtccaccggcggaagcgacatcttgctgactcagtctccaggcatcctgtctgtgagtccaggagaaagagtcagtttctcctgcagggccagacagagcattggcacagccatacactggtatcagcaaagaacaaatggttctccaaggcttctcataaagtatgcttctgagtctatctctgggatcccttccaggtttagcggcagtggatcagggacagattttactcttaccatcaacagtgtggagtctgaagatattgcagattattactgtcaacaaagtgggagctggccgtacacgttcggaggggggaccaagctggaaataaaa

SEQ ID NO: 393 - DNA, 729 bp, synthetic construct
  misc_feature 1..729: note = Description of Artificial Sequence: Synthetic polynucleotide
  source 1..729: mol_type = other DNA
gacatcttgctgactcagtctccaggcatcctgtctgtgagtccaggagaaagagtcagtttctcctgcagggccagacagagcattggcacagccatacactggtatcagcaaagaacaaatggttctccaaggcttctcataaagtatgcttctgagtctatctctgggatcccttccaggtttagcggcagtggatcagggacagattttactcttaccatcaacagtgtggagtctgaagatattgcagattattactgtcaacaaagtgggagctggccgtacacgttcggaggggggaccaagctggaaataaaaggcggatctgagggaaagtccagcggctccggcagcgaaagcaagtccaccggcggaagcgaggtgcaactggtggagtctgggggaggcctggtcaagcctggggggtccctgagactctcctgtgcagcctctggattcaccttcagtagatataacatgaactgggtccgccaggctccagggaaggggctggagtgggtctcatccattagtactagtagtaattacatatactacgcagactcagtgaagggccgattcaccttctccagagacaacgccaagaactcactggatctgcaaatgagcggcctgagagccgaggacacggctatttattactgtacgagaggctgggggccttttgactactggggccagggaaccctggtcaccgtctcctca

SEQ ID NO: 394 - DNA, 1425 bp, synthetic construct
  misc_feature 1..1425: note = Description of Artificial Sequence: Synthetic polynucleotide
  source 1..1425: mol_type = other DNA
gaggtgcagctggtggagagcggtggcggtctggtgaagccaggtggcagcctgcgcctgagctgtgccgccagcggtttcaccttcagccgctacaacatgaactgggtgcgccaagccccaggcaagggcctggagtgggtgagcagcatcagcaccagcagcaactacatctactacgccgacagcgtgaagggccgcttcaccttcagccgcgacaacgccaagaacagcctggacctgcagatgagcggtctgcgcgccgaggacaccgccatctactactgcacccgcggttggggcccattcgactactggggccagggcaccctggtgaccgtgagcagcggcggatctgagggaaagtccagcggctccggcagcgaaagcaagtccaccggcggaagcgacatccagatgacccagagcccaagcagcctgagcgccagcgtcggcgaccgcgtgaccatcacctgtcgtgcccgccagagcatcggcaccgccatccactggtaccagcagaagccaggcaaggccccaaagctgctgatctactacgccagcgagagcatcagcggtgtgccaagccgcttcagcggcagcggcagcggcaccgacttcaccctgaccatcagcagcgtgcagccagaggacttcgccacctactactgccagcagagcggcagctggccatacaccttcggccagggcaccaagctggagatcaaggagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacgtgtaccccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcgccctcgtgagcaagctcaccgtggacaagtctagatggcagcaggggaacgtcttctcatgctccgtgatgcatgaggctctgcacaaccactacacgcagaagagcctctccctgtctccgggtaaa

SEQ ID NO: 395 - DNA, 1425 bp, synthetic construct
  misc_feature 1..1425: note = Description of Artificial Sequence: Synthetic polynucleotide
  source 1..1425: mol_type = other DNA
gacatccagatgacccagagcccaagcagcctgagcgccagcgtcggcgaccgcgtgaccatcacctgtcgtgcccgccagagcatcggcaccgccatccactggtaccagcagaagccaggcaaggccccaaagctgctgatctactacgccagcgagagcatcagcggtgtgccaagccgcttcagcggcagcggcagcggcaccgacttcaccctgaccatcagcagcgtgcagccagaggacttcgccacctactactgccagcagagcggcagctggccatacaccttcggccagggcaccaagctggagatcaagggcggatctgagggaaagtccagcggctccggcagcgaaagcaagtccaccggcggaagcgaggtgcagctggtggagagcggtggcggtctggtgaagccaggtggcagcctgcgcctgagctgtgccgccagcggtttcaccttcagccgctacaacatgaactgggtgcgccaagccccaggcaagggcctggagtgggtgagcagcatcagcaccagcagcaactacatctactacgccgacagcgtgaagggccgcttcaccttcagccgcgacaacgccaagaacagcctggacctgcagatgagcggtctgcgcgccgaggacaccgccatctactactgcacccgcggttggggcccattcgactactggggccagggcaccctggtgaccgtgagcagcgagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacgtgtaccccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcgccctcgtgagcaagctcaccgtggacaagtctagatggcagcaggggaacgtcttctcatgctccgtgatgcatgaggctctgcacaaccactacacgcagaagagcctctccctgtctccgggtaaa

SEQ ID NO: 396 - DNA, 1425 bp, synthetic construct
  misc_feature 1..1425: note = Description of Artificial Sequence: Synthetic polynucleotide
  source 1..1425: mol_type = other DNA
gaggtgcagctggtggagagcggtggcggtctggtgaagccaggtggcagcctgcgcctgagctgtgccgccagcggtttcaccttcagccgctacaacatgaactgggtgcgccaagccccaggcaagggcctggagtgggtgagcagcatcagcaccagcagcaactacatctactacgccgacagcgtgaagggccgcttcaccttcagccgcgacaacgccaagaacagcctggacctgcagatgagcggtctgcgcgccgaggacaccgccatctactactgcacccgcggttggggcccattcgactactggggccagggcaccctggtgaccgtgagcagcggcggatctgagggaaagtccagcggctccggcagcgaaagcaagtccaccggcggaagcgacatccagatgacccagagcccaagcagcctgagcgccagcgtcggcgaccgcgtgaccatcacctgtcgtgcccgccagagcatcggcaccgccatccactggtaccagcagaagccaggcaaggccccaaagctgctgatcaagtacgccagcgagagcatcagcggtgtgccaagccgcttcagcggcagcggcagcggcaccgacttcaccctgaccatcagcagcctgcagccagaggacttcgccacctactactgccagcagagcggcagctggccatacaccttcggccagggcaccaagctggagatcaaggagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacgtgtaccccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcgccctcgtgagcaagctcaccgtggacaagtctagatggcagcaggggaacgtcttctcatgctccgtgatgcatgaggctctgcacaaccactacacgcagaagagcctctccctgtctccgggtaaa

SEQ ID NO: 397 - DNA, 1425 bp, synthetic construct
  misc_feature 1..1425: note = Description of Artificial Sequence: Synthetic polynucleotide
  source 1..1425: mol_type = other DNA
gacatccagatgacccagagcccaagcagcctgagcgccagcgtcggcgaccgcgtgaccatcacctgtcgtgcccgccagagcatcggcaccgccatccactggtaccagcagaagccaggcaaggccccaaagctgctgatcaagtacgccagcgagagcatcagcggtgtgccaagccgcttcagcggcagcggcagcggcaccgacttcaccctgaccatcagcagcctgcagccagaggacttcgccacctactactgccagcagagcggcagctggccatacaccttcggccagggcaccaagctggagatcaagggcggatctgagggaaagtccagcggctccggcagcgaaagcaagtccaccggcggaagcgaggtgcagctggtggagagcggtggcggtctggtgaagccaggtggcagcctgcgcctgagctgtgccgccagcggtttcaccttcagccgctacaacatgaactgggtgcgccaagccccaggcaagggcctggagtgggtgagcagcatcagcaccagcagcaactacatctactacgccgacagcgtgaagggccgcttcaccttcagccgcgacaacgccaagaacagcctggacctgcagatgagcggtctgcgcgccgaggacaccgccatctactactgcacccgcggttggggcccattcgactactggggccagggcaccctggtgaccgtgagcagcgagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacgtgtaccccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcgccctcgtgagcaagctcaccgtggacaagtctagatggcagcaggggaacgtcttctcatgctccgtgatgcatgaggctctgcacaaccactacacgcagaagagcctctccctgtctccgggtaaa

SEQ ID NO: 398 - DNA, 1425 bp, synthetic construct
  misc_feature 1..1425: note = Description of Artificial Sequence: Synthetic polynucleotide
  source 1..1425: mol_type = other DNA
gaggtgcagctggtggagagcggtggcggtctggtgaagccaggtggcagcctgcgcctgagctgtgccgccagcggtttcaccttcagccgctacaacatgaactgggtgcgccaagccccaggcaagggcctggagtgggtgagcagcatcagcaccagcagcaactacatctactacgccgacagcgtgaagggccgcttcaccttcagccgcgacaacgccaagaacagcctggacctgcagatgagcggtctgcgcgccgaggacaccgccatctactactgcacccgcggttggggcccattcgactactggggccagggcaccctggtgaccgtgagcagcggcggatctgagggaaagtccagcggctccggcagcgaaagcaagtccaccggcggaagcgacatccagatgacccagagcccaagcagcctgagcgccagcgtcggcgaccgcgtgaccatcacctgtcgtgcccgccagagcatcggcaccgccatccactggtaccagcagaagccaggcaaggccccaaagctgctgatcaagtacgccagcgagagcatcagcggtgtgccaagccgcttcagcggcagcggcagcggcaccgacttcaccctgaccatcagcagcgtgcagccagaggacttcgccacctactactgccagcagagcggcagctggccatacaccttcggccagggcaccaagctggagatcaaggagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacgtgtaccccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcgccctcgtgagcaagctcaccgtggacaagtctagatggcagcaggggaacgtcttctcatgctccgtgatgcatgaggctctgcacaaccactacacgcagaagagcctctccctgtctccgggtaaa

SEQ ID NO: 399 - DNA, 1425 bp, synthetic construct
  misc_feature 1..1425: note = Description of Artificial Sequence: Synthetic polynucleotide
  source 1..1425: mol_type = other DNA
gacatccagatgacccagagcccaagcagcctgagcgccagcgtcggcgaccgcgtgaccatcacctgtcgtgcccgccagagcatcggcaccgccatccactggtaccagcagaagccaggcaaggccccaaagctgctgatcaagtacgccagcgagagcatcagcggtgtgccaagccgcttcagcggcagcggcagcggcaccgacttcaccctgaccatcagcagcgtgcagccagaggacttcgccacctactactgccagcagagcggcagctggccatacaccttcggccagggcaccaagctggagatcaagggcggatctgagggaaagtccagcggctccggcagcgaaagcaagtccaccggcggaagcgaggtgcagctggtggagagcggtggcggtctggtgaagccaggtggcagcctgcgcctgagctgtgccgccagcggtttcaccttcagccgctacaacatgaactgggtgcgccaagccccaggcaagggcctggagtgggtgagcagcatcagcaccagcagcaactacatctactacgccgacagcgtgaagggccgcttcaccttcagccgcgacaacgccaagaacagcctggacctgcagatgagcggtctgcgcgccgaggacaccgccatctactactgcacccgcggttggggcccattcgactactggggccagggcaccctggtgaccgtgagcagcgagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacgtgtaccccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcgccctcgtgagcaagctcaccgtggacaagtctagatggcagcaggggaacgtcttctcatgctccgtgatgcatgaggctctgcacaaccactacacgcagaagagcctctccctgtctccgggtaaa

SEQ ID NO: 400 - DNA, 1425 bp, synthetic construct
  misc_feature 1..1425: note = Description of Artificial Sequence: Synthetic polynucleotide
  source 1..1425: mol_type = other DNA
gaggtgcagctggtggagagcggtggcggtctggtgaagccaggtggcagcctgcgcctgagctgtgccgccagcggtttcaccttcagccgctacaacatgaactgggtgcgccaagccccaggcaagggcctggagtgggtgagcagcatcagcaccagcagcaactacatctactacgccgacagcgtgaagggccgcttcaccttcagccgcgacaacgccaagaacagcctggacctgcagatgagcggtctgcgcgccgaggacaccgccatctactactgcacccgcggttggggcccattcgactactggggccagggcaccctggtgaccgtgagcagcggcggatctgagggaaagtccagcggctccggcagcgaaagcaagtccaccggcggaagcgacatccagatgacccagagcccaagcagcctgagcgccagcgtcggcgaccgcgtgaccatcacctgtcgtgcccgccagagcatcggcaccgccatccactggtaccagcagaagccaggcaaggccccaaagctgctgatctactacgccagcgagagcatcagcggtgtgccaagccgcttcagcggcagcggcagcggcaccgacttcaccctgaccatcagcagcctgcagccagaggacttcgccacctactactgccagcagagcggcagctggccatacaccttcggccagggcaccaagctggagatcaaggagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacgtgtaccccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcgccctcgtgagcaagctcaccgtggacaagtctagatggcagcaggggaacgtcttctcatgctccgtgatgcatgaggctctgcacaaccactacacgcagaagagcctctccctgtctccgggtaaa

SEQ ID NO: 401 - DNA, 1425 bp, synthetic construct
  misc_feature 1..1425: note = Description of Artificial Sequence: Synthetic polynucleotide
  source 1..1425: mol_type = other DNA
gacatccagatgacccagagcccaagcagcctgagcgccagcgtcggcgaccgcgtgaccatcacctgtcgtgcccgccagagcatcggcaccgccatccactggtaccagcagaagccaggcaaggccccaaagctgctgatctactacgccagcgagagcatcagcggtgtgccaagccgcttcagcggcagcggcagcggcaccgacttcaccctgaccatcagcagcctgcagccagaggacttcgccacctactactgccagcagagcggcagctggccatacaccttcggccagggcaccaagctggagatcaagggcggatctgagggaaagtccagcggctccggcagcgaaagcaagtccaccggcggaagcgaggtgcagctggtggagagcggtggcggtctggtgaagccaggtggcagcctgcgcctgagctgtgccgccagcggtttcaccttcagccgctacaacatgaactgggtgcgccaagccccaggcaagggcctggagtgggtgagcagcatcagcaccagcagcaactacatctactacgccgacagcgtgaagggccgcttcaccttcagccgcgacaacgccaagaacagcctggacctgcagatgagcggtctgcgcgccgaggacaccgccatctactactgcacccgcggttggggcccattcgactactggggccagggcaccctggtgaccgtgagcagcgagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacgtgtaccccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcgccctcgtgagcaagctcaccgtggacaagtctagatggcagcaggggaacgtcttctcatgctccgtgatgcatgaggctctgcacaaccactacacgcagaagagcctctccctgtctccgggtaaa

SEQ ID NO: 402 - DNA, 1425 bp, synthetic construct
  misc_feature 1..1425: note = Description of Artificial Sequence: Synthetic polynucleotide
  source 1..1425: mol_type = other DNA
gaggtgcaactggtggagtctgggggaggcctggtcaagcctggggggtccctgagactctcctgtgcagcctctggattcaccttcagtagatataacatgaactgggtccgccaggctccagggaaggggctggagtgggtctcatccattagtactagtagtaattacatatactacgcagactcagtgaagggccgattcaccttctccagagacaacgccaagaactcactggatctgcaaatgagcggcctgagagccgaggacacggctatttattactgtacgagaggctgggggccttttgactactggggccagggaaccctggtcaccgtctcctcaggcggatctgagggaaagtccagcggctccggcagcgaaagcaagtccaccggcggaagcgacatcttgctgactcagtctccaggcatcctgtctgtgagtccaggagaaagagtcagtttctcctgcagggccagacagagcattggcacagccatacactggtatcagcaaagaacaaatggttctccaaggcttctcataaagtatgcttctgagtctatctctgggatcccttccaggtttagcggcagtggatcagggacagattttactcttaccatcaacagtgtggagtctgaagatattgcagattattactgtcaacaaagtgggagctggccgtacacgttcggaggggggaccaagctggaaataaaagagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacgtgtaccccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcgccctcgtgagcaagctcaccgtggacaagtctagatggcagcaggggaacgtcttctcatgctccgtgatgcatgaggctctgcacaaccactacacgcagaagagcctctccctgtctccgggtaaa

SEQ ID NO: 403 - DNA, 1425 bp, synthetic construct
  misc_feature 1..1425: note = Description of Artificial Sequence: Synthetic polynucleotide
  source 1..1425: mol_type = other DNA
gacatcttgctgactcagtctccaggcatcctgtctgtgagtccaggagaaagagtcagtttctcctgcagggccagacagagcattggcacagccatacactggtatcagcaaagaacaaatggttctccaaggcttctcataaagtatgcttctgagtctatctctgggatcccttccaggtttagcggcagtggatcagggacagattttactcttaccatcaacagtgtggagtctgaagatattgcagattattactgtcaacaaagtgggagctggccgtacacgttcggaggggggaccaagctggaaataaaaggcggatctgagggaaagtccagcggctccggcagcgaaagcaagtccaccggcggaagcgaggtgcaactggtggagtctgggggaggcctggtcaagcctggggggtccctgagactctcctgtgcagcctctggattcaccttcagtagatataacatgaactgggtccgccaggctccagggaaggggctggagtgggtctcatccattagtactagtagtaattacatatactacgcagactcagtgaagggccgattcaccttctccagagacaacgccaagaactcactggatctgcaaatgagcggcctgagagccgaggacacggctatttattactgtacgagaggctgggggccttttgactactggggccagggaaccctggtcaccgtctcctcagagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacgtgtaccccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcgccctcgtgagcaagctcaccgtggacaagtctagatggcagcaggggaacgtcttctcatgctccgtgatgcatgaggctctgcacaaccactacacgcagaagagcctctccctgtctccgggtaaa

SEQ ID NO: 404 - protein, 252 aa, synthetic construct
  REGION 1..252: note = Description of Artificial Sequence: Synthetic polypeptide
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSSGGSEGKSSGSGSESKSTGGSDIQMTQSPSFLSASVGDRVTITCRASQGISSYLAWYQQKPGKAPKFLIYAASTLQSGVPSRFSGSGSGTEFTLTISSLQPEDFATYYCQQLNSYPLTFGGGTKVEIK

SEQ ID NO: 405 - protein, 252 aa, synthetic construct
  REGION 1..252: note = Description of Artificial Sequence: Synthetic polypeptide
DIQMTQSPSFLSASVGDRVTITCRASQGISSYLAWYQQKPGKAPKFLIYAASTLQSGVPSRFSGSGSGTEFTLTISSLQPEDFATYYCQQLNSYPLTFGGGTKVEIKGGSEGKSSGSGSESKSTGGSQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSS

SEQ ID NO: 406 - protein, 250 aa, synthetic construct
  REGION 1..250: note = Description of Artificial Sequence: Synthetic polypeptide
EVQLVESGGGVVQPGRSLRLSCVASGFTFSSYDIHWVRQAPGKGLEWVAIISYDGSKKDYTDSVKGRFTISRDNSKNTLYLQMDSLRVEDSAVYSCARESGWSHYYYYGMDVWGQGTMVTVSSGGSEGKSSGSGSESKSTGGSDIVMTQSPSSLSASVGDRVTITCRASQDISNYLAWYQQKPGKVPKFLIYAASTLHSGVPSRFSGSGSGTDFTLTISSLQPEDVATYYCQKYNSAPYTFGQGTRLEIK

SEQ ID NO: 407 - protein, 250 aa, synthetic construct
  REGION 1..250: note = Description of Artificial Sequence: Synthetic polypeptide
DIVMTQSPSSLSASVGDRVTITCRASQDISNYLAWYQQKPGKVPKFLIYAASTLHSGVPSRFSGSGSGTDFTLTISSLQPEDVATYYCQKYNSAPYTFGQGTRLEIKGGSEGKSSGSGSESKSTGGSEVQLVESGGGVVQPGRSLRLSCVASGFTFSSYDIHWVRQAPGKGLEWVAIISYDGSKKDYTDSVKGRFTISRDNSKNTLYLQMDSLRVEDSAVYSCARESGWSHYYYYGMDVWGQGTMVTVSS

SEQ ID NO: 408 - protein, 245 aa, synthetic construct
  REGION 1..245: note = Description of Artificial Sequence: Synthetic polypeptide
QVQLQESGPGLVKPSETLSLTCTVSGGSISSYYWSWLRQPAGSGLEWIGRLYVSGFTNYNPSLKSRVTLSLDPSRNQLSLKLSSVTAADTAVYYCAGDSGNYWGWFDPWGQGTLVTVSSGGSEGKSSGSGSESKSTGGSSYELTQPPSVSVSPGETASITCSGDQLGENYACWYQQKPGQSPVLVIYQDSKRPSGIPERFSGSNSGNTATLTISGTQALDEADYYCQAWDNSIVVFGGGTKLTVL

SEQ ID NO: 409 - protein, 245 aa, synthetic construct
  REGION 1..245: note = Description of Artificial Sequence: Synthetic polypeptide
SYELTQPPSVSVSPGETASITCSGDQLGENYACWYQQKPGQSPVLVIYQDSKRPSGIPERFSGSNSGNTATLTISGTQALDEADYYCQAWDNSIVVFGGGTKLTVLGGSEGKSSGSGSESKSTGGSQVQLQESGPGLVKPSETLSLTCTVSGGSISSYYWSWLRQPAGSGLEWIGRLYVSGFTNYNPSLKSRVTLSLDPSRNQLSLKLSSVTAADTAVYYCAGDSGNYWGWFDPWGQGTLVTVSS

SEQ ID NO: 410 - protein, 267 aa, synthetic construct
  REGION 1..267: note = Description of Artificial Sequence: Synthetic polypeptide
MAWVWTLLFLMAAAQSIQAQVQLQESGPGLVKPSQTLSLTCTVSGNSITSDYAWNWIRQFPGKGLEWIGYISYSGSTTYNPSLKSRVTISRDTSKNQFSLKLSSVTAADTAVYYCATGYYYGSGFWGQGTLVTVSSGGSEGKSSGSGSESKSTGGSDIVLTQSPDSLAVSLGERATINCRASESVEYFGTSLMHWYQQKPGQPPKLLIYAASNVESGVPDRFSGSGSGTDFTLTISSLQAEDVAVYFCQQTRKVPYTFGGGTKVEIK

SEQ ID NO: 411 - protein, 267 aa, synthetic construct
  REGION 1..267: note = Description of Artificial Sequence: Synthetic polypeptide
MAWVWTLLFLMAAAQSIQADIVLTQSPDSLAVSLGERATINCRASESVEYFGTSLMHWYQQKPGQPPKLLIYAASNVESGVPDRFSGSGSGTDFTLTISSLQAEDVAVYFCQQTRKVPYTFGGGTKVEIKGGSEGKSSGSGSESKSTGGSQVQLQESGPGLVKPSQTLSLTCTVSGNSITSDYAWNWIRQFPGKGLEWIGYISYSGSTTYNPSLKSRVTISRDTSKNQFSLKLSSVTAADTAVYYCATGYYYGSGFWGQGTLVTVSS

SEQ ID NO: 412 - protein, 267 aa, synthetic construct
  REGION 1..267: note = Description of Artificial Sequence: Synthetic polypeptide
MAWVWTLLFLMAAAQSIQAQVQLQESGPGLVKPSQTLSLTCTVSGNSITSDYAWNWIRQPPGKGLEWIGYISYSGSTTYNPSLKSRVTISRDTSKNQFSLKLSSVTAADTAVYYCATGYYYGSGFWGQGTLVTVSSGGSEGKSSGSGSESKSTGGSEIVLTQSPATLSLSPGERATLSCRASESVEYFGTSLMHWYQQKPGQPPRLLIYAASNVESGIPARFSGSGSGTDFTLTISSVEPEDFAVYFCQQTRKVPYTFGGGTKVEIK

SEQ ID NO: 413 - protein, 267 aa, synthetic construct
  REGION 1..267: note = Description of Artificial Sequence: Synthetic polypeptide
MAWVWTLLFLMAAAQSIQAEIVLTQSPATLSLSPGERATLSCRASESVEYFGTSLMHWYQQKPGQPPRLLIYAASNVESGIPARFSGSGSGTDFTLTISSVEPEDFAVYFCQQTRKVPYTFGGGTKVEIKGGSEGKSSGSGSESKSTGGSQVQLQESGPGLVKPSQTLSLTCTVSGNSITSDYAWNWIRQPPGKGLEWIGYISYSGSTTYNPSLKSRVTISRDTSKNQFSLKLSSVTAADTAVYYCATGYYYGSGFWGQGTLVTVSS

SEQ ID NO: 414 - protein, 267 aa, synthetic construct
  REGION 1..267: note = Description of Artificial Sequence: Synthetic polypeptide
MAWVWTLLFLMAAAQSIQAQVQLQESGPGLVKPSQTLSLTCTVSGNSITSDYAWNWIRQFPGKGLEWIGYISYSGSTTYNPSLKSRVTISRDTSKNQFSLKLSSVTAADTAVYYCATGYYYGSGFWGQGTLVTVSSGGSEGKSSGSGSESKSTGGSEIVLTQSPATLSLSPGERATLSCRASESVEYFGTSLMHWYQQKPGQPPRLLIYAASNVESGIPARFSGSGSGTDFTLTISSVEPEDFAVYFCQQTRKVPYTFGGGTKVEIK

SEQ ID NO: 415 - protein, 267 aa, synthetic construct
  REGION 1..267: note = Description of Artificial Sequence: Synthetic polypeptide
MAWVWTLLFLMAAAQSIQAEIVLTQSPATLSLSPGERATLSCRASESVEYFGTSLMHWYQQKPGQPPRLLIYAASNVESGIPARFSGSGSGTDFTLTISSVEPEDFAVYFCQQTRKVPYTFGGGTKVEIKGGSEGKSSGSGSESKSTGGSQVQLQESGPGLVKPSQTLSLTCTVSGNSITSDYAWNWIRQFPGKGLEWIGYISYSGSTTYNPSLKSRVTISRDTSKNQFSLKLSSVTAADTAVYYCATGYYYGSGFWGQGTLVTVSS

SEQ ID NO: 416 - protein, 271 aa, synthetic construct
  REGION 1..271: note = Description of Artificial Sequence: Synthetic polypeptide
MAWVWTLLFLMAAAQSIQAQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSSGGSEGKSSGSGSESKSTGGSDIQMTQSPSFLSASVGDRVTITCRASQGISSYLAWYQQKPGKAPKFLIYAASTLQSGVPSRFSGSGSGTEFTLTISSLQPEDFATYYCQQLNSYPLTFGGGTKVEIK

SEQ ID NO: 417 - protein, 271 aa, synthetic construct
  REGION 1..271: note = Description of Artificial Sequence: Synthetic polypeptide
MAWVWTLLFLMAAAQSIQADIQMTQSPSFLSASVGDRVTITCRASQGISSYLAWYQQKPGKAPKFLIYAASTLQSGVPSRFSGSGSGTEFTLTISSLQPEDFATYYCQQLNSYPLTFGGGTKVEIKGGSEGKSSGSGSESKSTGGSQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSS

SEQ ID NO: 418 - protein, 269 aa, synthetic construct
  REGION 1..269: note = Description of Artificial Sequence: Synthetic polypeptide
MAWVWTLLFLMAAAQSIQAEVQLVESGGGVVQPGRSLRLSCVASGFTFSSYDIHWVRQAPGKGLEWVAIISYDGSKKDYTDSVKGRFTISRDNSKNTLYLQMDSLRVEDSAVYSCARESGWSHYYYYGMDVWGQGTMVTVSSGGSEGKSSGSGSESKSTGGSDIVMTQSPSSLSASVGDRVTITCRASQDISNYLAWYQQKPGKVPKFLIYAASTLHSGVPSRFSGSGSGTDFTLTISSLQPEDVATYYCQKYNSAPYTFGQGTRLEIK

SEQ ID NO: 419 - protein, 269 aa, synthetic construct
  REGION 1..269: note = Description of Artificial Sequence: Synthetic polypeptide
MAWVWTLLFLMAAAQSIQADIVMTQSPSSLSASVGDRVTITCRASQDISNYLAWYQQKPGKVPKFLIYAASTLHSGVPSRFSGSGSGTDFTLTISSLQPEDVATYYCQKYNSAPYTFGQGTRLEIKGGSEGKSSGSGSESKSTGGSEVQLVESGGGVVQPGRSLRLSCVASGFTFSSYDIHWVRQAPGKGLEWVAIISYDGSKKDYTDSVKGRFTISRDNSKNTLYLQMDSLRVEDSAVYSCARESGWSHYYYYGMDVWGQGTMVTVSS

SEQ ID NO: 420 - protein, 264 aa, synthetic construct
  REGION 1..264: note = Description of Artificial Sequence: Synthetic polypeptide
MAWVWTLLFLMAAAQSIQAQVQLQESGPGLVKPSETLSLTCTVSGGSISSYYWSWLRQPAGSGLEWIGRLYVSGFTNYNPSLKSRVTLSLDPSRNQLSLKLSSVTAADTAVYYCAGDSGNYWGWFDPWGQGTLVTVSSGGSEGKSSGSGSESKSTGGSSYELTQPPSVSVSPGETASITCSGDQLGENYACWYQQKPGQSPVLVIYQDSKRPSGIPERFSGSNSGNTATLTISGTQALDEADYYCQAWDNSIVVFGGGTKLTVL

SEQ ID NO: 421 - protein, 264 aa, synthetic construct
  REGION 1..264: note = Description of Artificial Sequence: Synthetic polypeptide
MAWVWTLLFLMAAAQSIQASYELTQPPSVSVSPGETASITCSGDQLGENYACWYQQKPGQSPVLVIYQDSKRPSGIPERFSGSNSGNTATLTISGTQALDEADYYCQAWDNSIVVFGGGTKLTVLGGSEGKSSGSGSESKSTGGSQVQLQESGPGLVKPSETLSLTCTVSGGSISSYYWSWLRQPAGSGLEWIGRLYVSGFTNYNPSLKSRVTLSLDPSRNQLSLKLSSVTAADTAVYYCAGDSGNYWGWFDPWGQGTLVTVSS

SEQ ID NO: 422 - protein, 268 aa, synthetic construct
  REGION 1..268: note = Description of Artificial Sequence: Synthetic polypeptide
MAWVWTLLFLMAAAQSIQAQVQLVESGGGVVQPGRSLRLSCAASGFTFSYYGMHWVRQAPGKGLEWVAFISYDGSNKYYADSVKGRFTISRDNSKNTLYLQMNSLRAEDTAVYYCAHLPYSGSYWAFDYWGQGTQVTVSSGGSEGKSSGSGSESKSTGGSQSVLTQPPSVSVAPGQTASITCGGDNIGSKSVHWYQQKPGQAPVLVVYDNSDRPSGIPERFSGSNSGTTATLTISRVEAGDEADYYCQVWDSSSDHPVVFGGGTKVTV

SEQ ID NO: 423 - protein, 287 aa, synthetic construct
  REGION 1..287: note = Description of Artificial Sequence: Synthetic polypeptide
MAWVWTLLFLMAAAQSIQAQSVLTQPPSVSVAPGQTASITCGGDNIGSKSVHWYQQKPGQAPVLVVYDNSDRPSGIPERFSGSNSGTTATLTISRVEAGDEADYYCQVWDSSSDHPVVFGGGTKVTVGGSEGKSSGSGSESKSTGGSMAWVWTLLFLMAAAQSIQAQVQLVESGGGVVQPGRSLRLSCAASGFTFSYYGMHWVRQAPGKGLEWVAFISYDGSNKYYADSVKGRFTISRDNSKNTLYLQMNSLRAEDTAVYYCAHLPYSGSYWAFDYWGQGTQVTVSS

SEQ ID NO: 424 - protein, 253 aa, synthetic construct; identical to SEQ ID NO: 308
  REGION 1..253: note = Description of Artificial Sequence: Synthetic polypeptide

SEQ ID NO: 425 - protein, 272 aa, synthetic construct
  REGION 1..272: note = Description of Artificial Sequence: Synthetic polypeptide
MAWVWTLLFLMAAAQSIQASSELTQPPSVSVAPGQTARITCGGNNIGSKSVHWYQQKPGQAPVLVVYDDSDRPSGIPERFSGSNSGNTATLTISRVEAGDEADYYCQVWDSSSDHVVFGGGTKLTVLGGSEGKSSGSGSESKSTGGSQVQLVESGGGLVQPGGSLRLSCAASGFTFSHYAMSWVRQAPGKGLEWVSTIGGSGGSTYYADSVKGRFTISRDNSKNTLYLQMNSLRAEDTAVYYCAKPHIVMVTALLYDGMDVWGQGTMVTVSS

SEQ ID NO: 426 - protein, 471 aa, synthetic construct
  REGION 1..471: note = Description of Artificial Sequence: Synthetic polypeptide
QVQLQESGPGLVKPSQTLSLTCTVSGNSITSDYAWNWIRQFPGKGLEWIGYISYSGSTTYNPSLKSRVTISRDTSKNQFSLKLSSVTAADTAVYYCATGYYYGSGFWGQGTLVTVSSGGSEGKSSGSGSESKSTGGSDIVLTQSPDSLAVSLGERATINCRASESVEYFGTSLMHWYQQKPGQPPKLLIYAASNVESGVPDRFSGSGSGTDFTLTISSLQAEDVAVYFCQQTRKVPYTFGGGTKVEIKTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 427 - protein, 471 aa, synthetic construct
  REGION 1..471: note = Description of Artificial Sequence: Synthetic polypeptide
DIVLTQSPDSLAVSLGERATINCRASESVEYFGTSLMHWYQQKPGQPPKLLIYAASNVESGVPDRFSGSGSGTDFTLTISSLQAEDVAVYFCQQTRKVPYTFGGGTKVEIKGGSEGKSSGSGSESKSTGGSQVQLQESGPGLVKPSQTLSLTCTVSGNSITSDYAWNWIRQFPGKGLEWIGYISYSGSTTYNPSLKSRVTISRDTSKNQFSLKLSSVTAADTAVYYCATGYYYGSGFWGQGTLVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 428 - protein, 471 aa, synthetic construct
  REGION 1..471: note = Description of Artificial Sequence: Synthetic polypeptide
QVQLQESGPGLVKPSQTLSLTCTVSGNSITSDYAWNWIRQPPGKGLEWIGYISYSGSTTYNPSLKSRVTISRDTSKNQFSLKLSSVTAADTAVYYCATGYYYGSGFWGQGTLVTVSSGGSEGKSSGSGSESKSTGGSEIVLTQSPATLSLSPGERATLSCRASESVEYFGTSLMHWYQQKPGQPPRLLIYAASNVESGIPARFSGSGSGTDFTLTISSVEPEDFAVYFCQQTRKVPYTFGGGTKVEIKTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 429 - protein, 471 aa, synthetic construct; identical to SEQ ID NO: 428
  REGION 1..471: note = Description of Artificial Sequence: Synthetic polypeptide

SEQ ID NO: 430 - protein, 471 aa, synthetic construct
  REGION 1..471: note = Description of Artificial Sequence: Synthetic polypeptide
QVQLQESGPGLVKPSQTLSLTCTVSGNSITSDYAWNWIRQFPGKGLEWIGYISYSGSTTYNPSLKSRVTISRDTSKNQFSLKLSSVTAADTAVYYCATGYYYGSGFWGQGTLVTVSSGGSEGKSSGSGSESKSTGGSEIVLTQSPATLSLSPGERATLSCRASESVEYFGTSLMHWYQQKPGQPPRLLIYAASNVESGIPARFSGSGSGTDFTLTISSVEPEDFAVYFCQQTRKVPYTFGGGTKVEIKTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 431 - protein, 471 aa, synthetic construct; identical to SEQ ID NO: 430
  REGION 1..471: note = Description of Artificial Sequence: Synthetic polypeptide

SEQ ID NO: 432 - protein, 475 aa, synthetic construct
  REGION 1..475: note = Description of Artificial Sequence: Synthetic polypeptide
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSSGGSEGKSSGSGSESKSTGGSDIQMTQSPSFLSASVGDRVTITCRASQGISSYLAWYQQKPGKAPKFLIYAASTLQSGVPSRFSGSGSGTEFTLTISSLQPEDFATYYCQQLNSYPLTFGGGTKVEIKTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 433 - protein, 475 aa, synthetic construct
  REGION 1..475: note = Description of Artificial Sequence: Synthetic polypeptide
DIQMTQSPSFLSASVGDRVTITCRASQGISSYLAWYQQKPGKAPKFLIYAASTLQSGVPSRFSGSGSGTEFTLTISSLQPEDFATYYCQQLNSYPLTFGGGTKVEIKGGSEGKSSGSGSESKSTGGSQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 434 - protein, 473 aa, synthetic construct
  REGION 1..473: note = Description of Artificial Sequence: Synthetic polypeptide
EVQLVESGGGVVQPGRSLRLSCVASGFTFSSYDIHWVRQAPGKGLEWVAIISYDGSKKDYTDSVKGRFTISRDNSKNTLYLQMDSLRVEDSAVYSCARESGWSHYYYYGMDVWGQGTMVTVSSGGSEGKSSGSGSESKSTGGSDIVMTQSPSSLSASVGDRVTITCRASQDISNYLAWYQQKPGKVPKFLIYAASTLHSGVPSRFSGSGSGTDFTLTISSLQPEDVATYYCQKYNSAPYTFGQGTRLEIKTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 435 - protein, 473 aa, synthetic construct
  REGION 1..473: note = Description of Artificial Sequence: Synthetic polypeptide
DIVMTQSPSSLSASVGDRVTITCRASQDISNYLAWYQQKPGKVPKFLIYAASTLHSGVPSRFSGSGSGTDFTLTISSLQPEDVATYYCQKYNSAPYTFGQGTRLEIKGGSEGKSSGSGSESKSTGGSEVQLVESGGGVVQPGRSLRLSCVASGFTFSSYDIHWVRQAPGKGLEWVAIISYDGSKKDYTDSVKGRFTISRDNSKNTLYLQMDSLRVEDSAVYSCARESGWSHYYYYGMDVWGQGTMVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 436 - protein, 468 aa, synthetic construct
  REGION 1..468: note = Description of Artificial Sequence: Synthetic polypeptide
QVQLQESGPGLVKPSETLSLTCTVSGGSISSYYWSWLRQPAGSGLEWIGRLYVSGFTNYNPSLKSRVTLSLDPSRNQLSLKLSSVTAADTAVYYCAGDSGNYWGWFDPWGQGTLVTVSSGGSEGKSSGSGSESKSTGGSSYELTQPPSVSVSPGETASITCSGDQLGENYACWYQQKPGQSPVLVIYQDSKRPSGIPERFSGSNSGNTATLTISGTQALDEADYYCQAWDNSIVVFGGGTKLTVLTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 437 - protein, 468 aa, synthetic construct
  REGION 1..468: note = Description of Artificial Sequence: Synthetic polypeptide
SYELTQPPSVSVSPGETASITCSGDQLGENYACWYQQKPGQSPVLVIYQDSKRPSGIPERFSGSNSGNTATLTISGTQALDEADYYCQAWDNSIVVFGGGTKLTVLGGSEGKSSGSGSESKSTGGSQVQLQESGPGLVKPSETLSLTCTVSGGSISSYYWSWLRQPAGSGLEWIGRLYVSGFTNYNPSLKSRVTLSLDPSRNQLSLKLSSVTAADTAVYYCAGDSGNYWGWFDPWGQGTLVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 438 - protein, 472 aa, synthetic construct
  REGION 1..472: note = Description of Artificial Sequence: Synthetic polypeptide
QVQLVESGGGVVQPGRSLRLSCAASGFTFSYYGMHWVRQAPGKGLEWVAFISYDGSNKYYADSVKGRFTISRDNSKNTLYLQMNSLRAEDTAVYYCAHLPYSGSYWAFDYWGQGTQVTVSSGGSEGKSSGSGSESKSTGGSQSVLTQPPSVSVAPGQTASITCGGDNIGSKSVHWYQQKPGQAPVLVVYDNSDRPSGIPERFSGSNSGTTATLTISRVEAGDEADYYCQVWDSSSDHPVVFGGGTKVTVTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 439 - protein, 472 aa, synthetic construct
  REGION 1..472: note = Description of Artificial Sequence: Synthetic polypeptide
QSVLTQPPSVSVAPGQTASITCGGDNIGSKSVHWYQQKPGQAPVLVVYDNSDRPSGIPERFSGSNSGTTATLTISRVEAGDEADYYCQVWDSSSDHPVVFGGGTKVTVGGSEGKSSGSGSESKSTGGSQVQLVESGGGVVQPGRSLRLSCAASGFTFSYYGMHWVRQAPGKGLEWVAFISYDGSNKYYADSVKGRFTISRDNSKNTLYLQMNSLRAEDTAVYYCAHLPYSGSYWAFDYWGQGTQVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 440 - protein, 476 aa, synthetic construct
  REGION 1..476: note = Description of Artificial Sequence: Synthetic polypeptide
QVQLVESGGGLVQPGGSLRLSCAASGFTFSHYAMSWVRQAPGKGLEWVSTIGGSGGSTYYADSVKGRFTISRDNSKNTLYLQMNSLRAEDTAVYYCAKPHIVMVTALLYDGMDVWGQGTMVTVSSGGSEGKSSGSGSESKSTGGSSSELTQPPSVSVAPGQTARITCGGNNIGSKSVHWYQQKPGQAPVLVVYDDSDRPSGIPERFSGSNSGNTATLTISRVEAGDEADYYCQVWDSSSDHVVFGGGTKLTVLTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 441 - protein, 476 aa, synthetic construct
  REGION 1..476: note = Description of Artificial Sequence: Synthetic polypeptide
SSELTQPPSVSVAPGQTARITCGGNNIGSKSVHWYQQKPGQAPVLVVYDDSDRPSGIPERFSGSNSGNTATLTISRVEAGDEADYYCQVWDSSSDHVVFGGGTKLTVLGGSEGKSSGSGSESKSTGGSQVQLVESGGGLVQPGGSLRLSCAASGFTFSHYAMSWVRQAPGKGLEWVSTIGGSGGSTYYADSVKGRFTISRDNSKNTLYLQMNSLRAEDTAVYYCAKPHIVMVTALLYDGMDVWGQGTMVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 442 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 443 - protein, 104 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 444 - protein, 106 aa, synthetic construct
  REGION 1..106: note = Description of Artificial Sequence: Synthetic polypeptide
SYELTQPPSVSVSPGETASITCSGDQLGENYASWYQQKPGQSPVLVIYQDSKRPSGIPERFSGSNSGNTATLTISGTQALDEADYYCQAWDNSIVVFGGGTKLTVL

SEQ ID NO: 445 - protein, 212 aa, synthetic construct
  REGION 1..212: note = Description of Artificial Sequence: Synthetic polypeptide
SYELTQPPSVSVSPGETASITCSGDQLGENYASWYQQKPGQSPVLVIYQDSKRPSGIPERFSGSNSGNTATLTISGTQALDEADYYCQAWDNSIVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 446 - DNA, 636 bp, synthetic construct
  misc_feature 1..636: note = Description of Artificial Sequence: Synthetic polynucleotide
  source 1..636: mol_type = other DNA
agttatgagctgactcaaccacccagtgtcagcgtatccccaggagaaactgcctctataacatgcagcggagaccagttgggagaaaattacgcctcctggtaccaacagaagcctggacaaagtcctgtcctcgttatttatcaagattctaaacgtccctctgggatccccgaacgattctccggctctaactctgggaataccgctaccttgacaataagtggtacacaggcacttgatgaagctgattattactgccaggcatgggataacagcattgtggttttcgggggcggcaccaaactcacagttctcggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 447 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 448 - protein, 9 aa, Homo sapiens
GSDEDHLSL

SEQ ID NO: 449 - protein, 6 aa, Homo sapiens
PRGSKP

SEQ ID NO: 450 - DNA, 642 bp, synthetic construct
  misc_feature 1..642: note = Description of Artificial Sequence: Synthetic polynucleotide
  source 1..642: mol_type = other DNA
gacatccagatgacccagtctccttccttcctgtctgcatctgtaggagacagagtcaccatcacttgccgggccagtcagggcattagcagttatttagcctggtatcagcaaaaaccagggaaagcccctaagttcctgatctatgctgcatccactttgcaaagtggggtcccatcaaggttcagcggcagtggatctgggacagaattcactctcacaatcagcagcctgcagcctgaagattttgcaacttattactgtcaacagcttaatagttaccctctcactttcggcggagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 451 - DNA, 642 bp, synthetic construct
  misc_feature 1..642: note = Description of Artificial Sequence: Synthetic polynucleotide
  source 1..642: mol_type = other DNA
gacatcgtgatgacccagtctccatcctccctgtctgcatctgtaggagacagagtcaccatcacttgccgggcgagtcaggacattagcaattatttagcctggtatcagcagaaaccagggaaagttcctaagttcctgatctatgctgcatccactttgcactctggggtcccatctcggttcagtggcagtggatctgggacagatttcactctcaccatcagcagcctgcagcctgaagatgttgcaacttattactgtcaaaagtataacagtgccccgtacacttttggccaagggacacgactggagattaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 452 - DNA, 642 bp, synthetic construct
  misc_feature 1..642: note = Description of Artificial Sequence: Synthetic polynucleotide
  source 1..642: mol_type = other DNA
gacatccagatgacccagagcccaagcagcctgagcgccagcgtcggcgaccgcgtgaccatcacctgtcgtgcccgccagagcatcggcaccgccatccactggtaccagcagaagccaggcaaggccccaaagctgctgatcaagtacgccagcgagagcatcagcggtgtgccaagccgcttcagcggcagcggcagcggcaccgacttcaccctgaccatcagcagcctgcagccagaggacttcgccacctactactgccagcagagcggcagctggccatacaccttcggccagggcaccaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 453 - protein, 475 aa, synthetic construct
  REGION 1..475: note = Description of Artificial Sequence: Synthetic polypeptide
DIQMTQSPSSLSASVGDRVTITCRARQSIGTAIHWYQQKPGKAPKLLIKYASESISGVPSRFSGSGSGTDFTLTISSVQPEDFATYYCQQSGSWPYTFGQGTKLEIKGGSEGKSSGSGSESKSTGGSEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 454 - protein, 240 aa, synthetic construct
  REGION 1..240: note = Description of Artificial Sequence: Synthetic polypeptide
VPLIEGRIVGGWECEKHSQPWQVAVYSHGWAHCGGVLVHPQWVLTAAHCLKKNSQVWLGRHNLFEPEDTGQRVPVSHSFPHPLYNMSLLKHQSLRPDEDSSHDLMLLRLSEPAKITDVVKVLGLPTQEPALGTTCYASGWGSIEPEEFLRPRSLQCVSLHYSEKVTEFMLCAGLWTGGKDTCGGDSGGPLVCNGVLQGITSWGPEPCALPEKPAVYTKVVHYRKWIKDTIAANPHHHHHH

SEQ ID NO: 455 - protein, 6 aa, synthetic construct
  REGION 1..6: note = Description of Artificial Sequence: Synthetic peptide
GFTFSH

SEQ ID NO: 456 - protein, 17 aa, synthetic construct
  REGION 1..17: note = Description of Artificial Sequence: Synthetic peptide
TAVYYCAKPHIVMVTAL

SEQ ID NO: 457 - protein, 13 aa, synthetic construct
  REGION 1..13: note = Description of Artificial Sequence: Synthetic peptide
YDDSDRPSGIPER

SEQ ID NO: 458 - protein, 5 aa, synthetic construct
  REGION 1..5: note = Description of Artificial Sequence: Synthetic peptide
FTFSY

SEQ ID NO: 459 - protein, 8 aa, synthetic construct
  REGION 1..8: note = Description of Artificial Sequence: Synthetic peptide
GSYWAFDY

SEQ ID NO: 460 - protein, 4 aa, synthetic construct
  REGION 1..4: note = Description of Artificial Sequence: Synthetic peptide
DNSD

SEQ ID NO: 461 - protein, 9 aa, synthetic construct
  REGION 1..9: note = Description of Artificial Sequence: Synthetic peptide
GNSITSDYA

SEQ ID NO: 462 - protein, 4 aa, synthetic construct
  REGION 1..4: note = Description of Artificial Sequence: Synthetic peptide
GFTF

SEQ ID NO: 463 - protein, 9 aa, synthetic construct
  REGION 1..9: note = Description of Artificial Sequence: Synthetic peptide
ARDQNYDIL

SEQ ID NO: 464 - protein, 5 aa, synthetic construct
  REGION 1..5: note = Description of Artificial Sequence: Synthetic peptide
VTPNF

SEQ ID NO: 465 - protein, 9 aa, synthetic construct
  REGION 1..9: note = Description of Artificial Sequence: Synthetic peptide
YAASTLQSG

SEQ ID NO: 466 - protein, 7 aa, synthetic construct
  REGION 1..7: note = Description of Artificial Sequence: Synthetic peptide
ESGWSHY

SEQ ID NO: 467 - protein, 244 aa, synthetic construct
  REGION 1..244: note = Description of Artificial Sequence: Synthetic polypeptide
VPLIEGRIVGGWECEKHSQPWQVAVYSHGWAHCGGVLVHPQWVLTAAHCLKKNSQVWLGRHNLFEPEDTGQRVPVSHSFPHPLYNMSLLKHQSLRPDEDSSHDLMLLRLSEPAKITDVVKVLGLPTQEPALGTTCYASGWGSIEPEEFLRPRSLQCVSLHLLSNDMCARAYSEKVTEFMLCAGLWTGGKDTCGGDSGGPLVCNGVLQGITSWGPEPCALPEKPAVYTKVVHYRKWIKDTIAANP

SEQ ID NO: 468 - protein, 451 aa, synthetic construct
  REGION 1..451: note = Description of Artificial Sequence: Synthetic polypeptide
EVQLVESGGGLVQPGGSLRLSCAASGFTFSSYWMTWVRQPPGKGLEWVANIKQDGSERYFVDSVKGRFTISRDNAKNSLYLQMNSLRAEDTAVYYCARDQNYDILTGHYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 469 - protein, 214 aa, synthetic construct
  REGION 1..214: note = Description of Artificial Sequence: Synthetic polypeptide
EIVLTQSPSFLSASVGDRVTITCRASQGISSYLSWYQQKPGKAPKLLIYATSTLQSGVPSRFSGSGSGTEFTLTISSLQPEDFATYYCQQLNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 470 - protein, 7 aa, synthetic construct
  REGION 1..7: note = Description of Artificial Sequence: Synthetic peptide
DDSDRPS

SEQ ID NO: 471 - DNA, 1338 bp, synthetic construct
  misc_feature 1..1338: note = Description of Artificial Sequence: Synthetic polynucleotide
  source 1..1338: mol_type = other DNA
gaggtgcagctggtggagagcggtggcggtctggtgaagccaggtggcagcctgcgcctgagctgtgccgccagcggtttcaccttcagccgctacaacatgaactgggtgcgccaagccccaggcaagggcctggagtgggtgagcagcatcagcaccagcagcaactacatctactacgccgacagcgtgaagggccgcttcaccttcagccgcgacaacgccaagaacagcctggacctgcagatgagcggtctgcgcgccgaggacaccgccatctactactgcacccgcggttggggcccattcgactactggggccagggcaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacgtgtaccccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcgccctcgtgagcaagctcaccgtggacaagtctagatggcagcaggggaacgtcttctcatgctccgtgatgcatgaggctctgcacaaccactacacgcagaagagcctctccctgtctccgggtaaa

SEQ ID NO: 472 - protein, 254 aa, synthetic construct
  REGION 1..254: note = KL2B997 HC
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGSGSGSHHHHHHGSGLPETGG

SEQ ID NO: 473 - protein, 214 aa, synthetic construct; identical to SEQ ID NO: 221
  REGION 1..214: note = KL2B997 LC

SEQ ID NO: 474 - protein, 228 aa, synthetic construct
  REGION 1..228: note = KL2B1251 HC
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 475 - protein, 214 aa, synthetic construct; identical to SEQ ID NO: 221
  REGION 1..214: note = KL2B1251 LC